(19) United States
(12) Patent Application Publication
(10) Pub. No.: US 2024/0239792 A1
(43) Pub. Date: Jul. 18, 2024

(54) MODULATORS OF BCL-2 OR BCL-2/BCL-XL AND USES THEREOF

(71) Applicant: APPICINE THERAPEUTICS (HK) LIMITED, Hong Kong (CN)

(72) Inventor: Zhengying PAN, Shenzhen (CN)

(21) Appl. No.: 18/555,250

(22) PCT Filed: Apr. 12, 2022
(86) PCT No.: PCT/CN2022/086388

§ 371 Date: Oct. 12, 2023

(30) Foreign Application Priority Data
Apr. 13, 2021 (WO) PCT/CN2021/087012
Apr. 10, 2022 (CN) 202210370576.1

(51) Int. Cl. C07D 471/04; A61K 31/496; A61K 31/5377; A61K 31/55; A61K 31/635; A61K 31/675; A61K 45/06; A61P 35/00; C07D 519/00

(52) U.S. Cl. CPC C07D 471/04; A61K 31/496; A61K 31/5377; A61K 31/55; A61K 31/635; A61K 31/675; A61K 45/06; A61P 35/00; C07D 519/00

(57) ABSTRACT

Compounds of Formula (I) which modulate the level or activity of BCL-2 protein or BCL-2/BCL-XL proteins, pharmaceutical compositions containing one or more of the compounds and the use thereof.

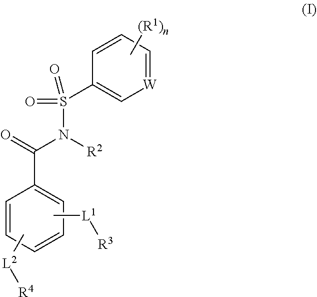

### FIELD OF THE DISCLOSURE

The present application relates to novel compounds, and tautomers, stereoisomers, or pharmaceutically acceptable salts thereof, which modulate the level or activity of BCL-2 protein or BCL-2/BCL-XL proteins. The present application also relates to pharmaceutical compositions comprising one or more of the compounds and tautomers, stereoisomers, or pharmaceutically acceptable salts thereof as an active ingredient, and to the use of the compounds and tautomers, stereoisomers, or pharmaceutically acceptable salts thereof in the treatment of BCL-2 protein or BCL-2/BCL-XL proteins associated diseases, disorders or conditions, including cancers.

### BACKGROUND OF THE DISCLOSURE

BCL-2 (B-cell lymphoma 2) protein, encoded in humans by the BCL2 gene, is the founding member of the BCL-2 family of regulator proteins that regulate cell death (apoptosis). B-cell lymphoma-extra large (BCL-XL), encoded by the BCL2-like 1 gene, is a transmembrane molecule in the mitochondria. BCL-XL is a member of the BCL-2 family of proteins, and acts as an anti-apoptotic protein by preventing the release of mitochondrial contents such as cytochrome c, which leads to caspase activation and ultimately, programmed cell death (S J Korsmeyer, “Regulators of Cell Death”, Trends in Genetics 11 (3): 101-105, March 1995).

A number of compounds have been reported by showing activity against BCL-2, for example, in WO2005/049593 (Abbot Laboratories), WO2010/138588 (Abbot Laboratories), etc. Venetoclax (Venclexta®/Venclyxto®), a selective BCL-2 inhibitor, has been approved by the U.S. Food and Drug Administration for treating chronic lymphocytic leukemia (CLL), small lymphocytic lymphoma (SLL), or acute myeloid leukemia (AML).

There remains a need to develop novel compounds that selectively inhibit the level or activity of BCL-2 protein, and novel compounds that inhibit the levels or activities of both BCL-2 and BCL-XL proteins.

### SUMMARY OF THE DISCLOSURE

Disclosed herein are novel compounds that possess potent BCL-2 selective inhibitory activity, or BCL-2/BCL-XL dual inhibitory activity. As a result, the compounds of the present application are particularly useful in the treatment of BCL-2 or BCL-2/BCL-XL associated diseases, disorders or conditions.

In one aspect, the present disclosure provides compounds of Formula I:

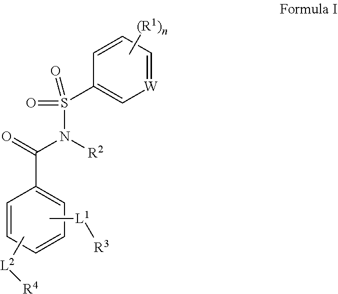

or tautomers, stereoisomers, or pharmaceutically acceptable salts thereof, wherein:
W is N or C(R¹);
n is 0, 1, 2 or 3;
each R¹ is independently selected from the group consisting of hydrogen, halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, and —NH-L³-R^{a}, wherein,
L³ is absent or selected from alkyl, alkenyl, or alkynyl, each of which is optionally substituted with one or more R^{b};
R^{a} is selected from the group consisting of cycloalkyl, heterocyclyl, aryl and heteroaryl, wherein each of the cycloalkyl, heterocyclyl, aryl and heteroaryl is optionally substituted with one or more R^{c};
R² is selected from the group consisting of hydrogen, alkyl, alkenyl, alkynyl, heteroalkyl, heteroalkenyl, heteroalkynyl, haloalkyl, and alkylalkoxyl; L¹ is absent, O, S, or N;
R³ is absent, cycloalkyl, heterocyclyl, aryl or heteroaryl, wherein each of cycloalkyl, heterocyclyl, aryl or heteroaryl is optionally substituted with one or more R^{d};
L² is selected from the group consisting of C₁₋₆ alkyl, C₁₋₆ alkenyl, C₁₋₆ alkynyl, halo-C₁₋₆ alkyl, hetero-C₁₋₆ alkenyl, hetero-C₁₋₆ alkynyl, cycloalkyl, heterocyclyl, aryl and heteroaryl, each of which is optionally substituted with one or more R^{e};
R⁴ is

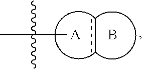

wherein
Ring A is selected from the group consisting of cycloalkyl, heterocyclyl, aryl, and heteroaryl, each of which is optionally substituted with one or more R^{f};
Ring B is selected from the group consisting of cycloalkyl, heterocyclyl, aryl, and heteroaryl, each of which is optionally substituted with one or more R^{g};
[symbol] is a bond via which Ring A is fused to Ring B;
each R^{c} is independently selected from the group consisting of halogen, cyano, hydroxyl, sulfhydryl, —NH₂, —NO₂, alkyl, alkenyl, alkynyl, heteroalkyl, heteroalkenyl, heteroalkynyl, haloalkyl, alkoxyl, haloalkoxyl, cycloalkyl, heterocyclyl, aryl, heteroaryl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3};
R^{b}, R^{d} and R^{e} are each independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl;
each R^{f} is independently selected from the group consisting of oxo, halogen, cyano, hydroxyl, sulfhydryl, —NH₂, —NO₂, alkyl, alkenyl, alkynyl, heteroalkyl, heteroalkenyl, heteroalkynyl, haloalkyl, alkoxyl, haloalkoxyl, cycloalkyl, heterocyclyl, aryl, heteroaryl, and —S(O)₂—R^{a4};
each R^{g} is independently selected from the group consisting of oxo, halogen, cyano, hydroxyl, sulfhydryl, —NH₂, —NO₂, alkyl, alkenyl, alkynyl, heteroalkyl, heteroalkenyl, heteroalkynyl, haloalkyl, alkoxyl, haloalkoxyl, cycloalkyl, heterocyclyl, aryl, heteroaryl, —NH—C(O)—R^{a5}, —NH—S(O)₂—R^{a5}, —P(O)(R^{a5})₂, —S(O)₂—R^{a5}, wherein each of alkyl, alkenyl, alkynyl, heteroalkyl, heteroalkenyl, heteroalkynyl, cycloalkyl, heterocyclyl, aryl, and heteroaryl is optionally substituted with one or more of a group selected from halogen, cyano, hydroxyl, sulfhydryl, —NH₂, —NO₂, alkyl, alkenyl, alkynyl, heteroalkyl, heteroalkenyl, or heteroalkynyl;
R^{a1}, R^{a2} and R^{a3} are each independently selected from the group consisting of hydrogen, hydroxyl, halogen, alkyl, haloalkyl, alkoxyl, cycloalkyl and -alkyl-NH₂;
R^{a4} and R^{as} are each independently selected from the group consisting of alkyl, alkenyl, alkynyl, heteroalkyl, heteroalkenyl, heteroalkynyl, cycloalkyl, heterocyclyl, aryl and heteroaryl, wherein each of cycloalkyl, heterocyclyl, aryl and heteroaryl is optionally substituted with one or more of a group selected from halogen, cyano, hydroxyl, sulfhydryl, —NH₂, —NO₂, alkyl, alkenyl, alkynyl, heteroalkyl, heteroalkenyl, or heteroalkynyl.

In another aspect, the present disclosure provides compounds of Formula II:

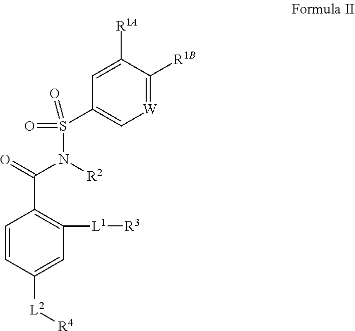

or tautomers, stereoisomers, or pharmaceutically acceptable salts thereof, wherein:
W is N or C(R¹);
R^{1A} is selected from the group consisting of hydrogen, halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, and haloalkoxyl;
R^{1B} is absent or —NH-L³-R^{a};
R¹, R², L¹, R³, L², R⁴, L³, R^{a} are each defined as supra.

In a further aspect, the present disclosure provides compounds of Formula III or Formula IV:

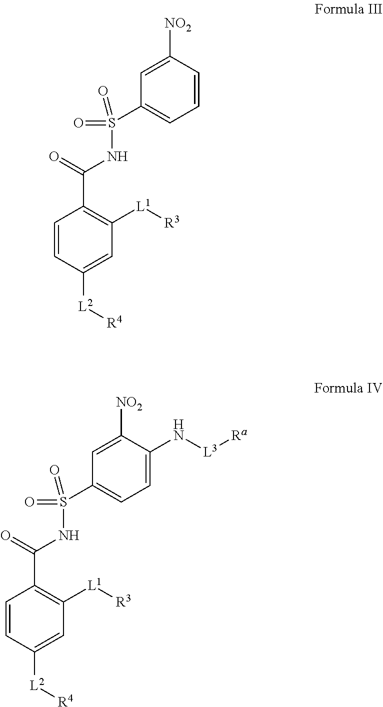

or tautomers, stereoisomers, or pharmaceutically acceptable salts thereof, wherein -L¹-R³ is absent or

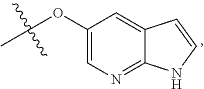 [SMILES: *Oc1cnc2[nH]ccc2c1]

L², L³, R^{a} and R⁴ are defined as supra.

In a further aspect, the present disclosure provides compounds of Formula IV(a), Formula IV(b), Formula IV(c), Formula IV(d), or Formula IV(e):

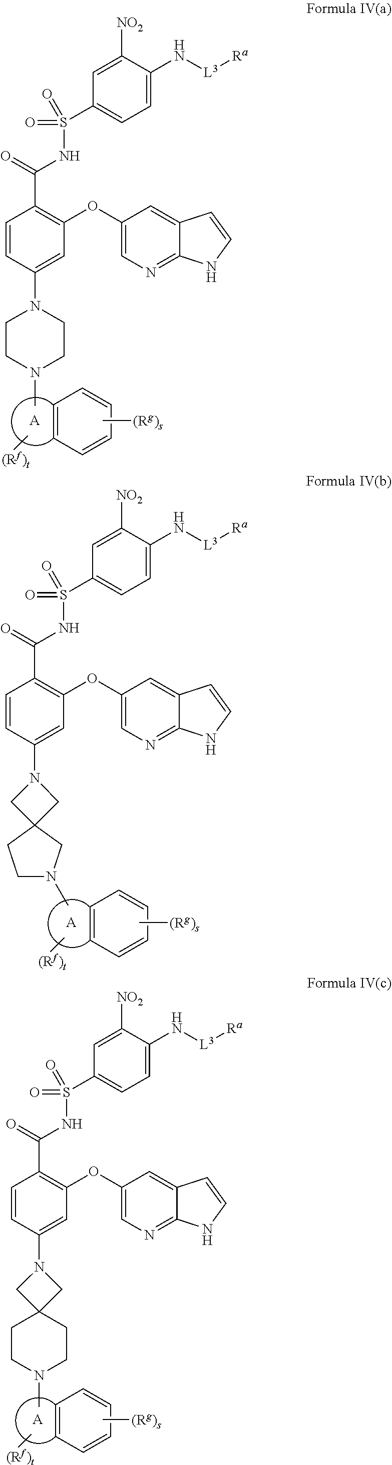

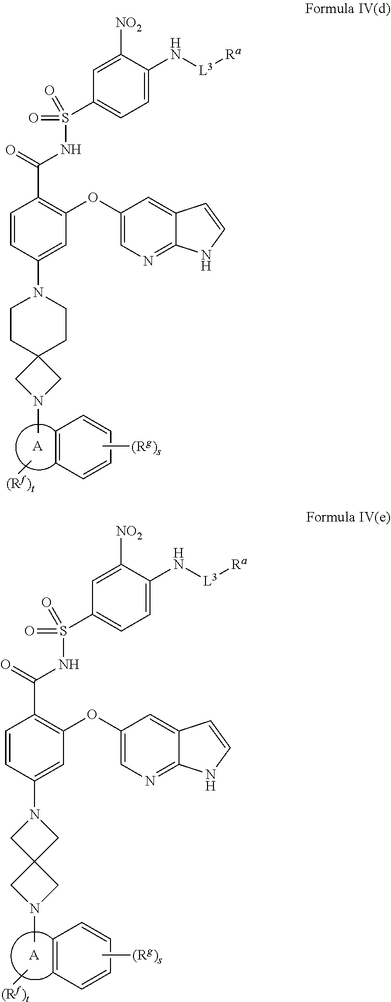

or tautomers, stereoisomers, or pharmaceutically acceptable salts thereof, wherein
L³, R^{a}, and Ring A are each defined as supra,
each R^{f} is independently oxo, alkyl, —S(O)₂-alkyl or —S(O)₂-phenyl, wherein the phenyl is optionally substituted with one or more alkyl;
each R^{g} is independently selected from the group consisting of hydroxyl, halogen, —NH₂, —NO₂, —NH—C(O)-alkyl, —NH—S(O)₂-alkyl, —P(O)(alkyl)₂, —S(O)₂-aryl, alkyl, alkenyl, cycloalkyl, aryl and heteroaryl, wherein each of alkyl, aryl and heteroaryl is optionally substituted with one or more group selected from hydroxyl, halogen or alkyl; and
each of s and t is independently 0, 1, 2 or 3.

In another aspect, the present disclosure provides a pharmaceutical composition comprising (i) the compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, and (ii) a pharmaceutically acceptable excipient or pharmaceutically acceptable carrier.

In a further aspect, the present disclosure provides a method of modulating the level or activity of BCL-2 protein or BCL-2/BCL-XL proteins in a cell, comprising exposing the cell to the compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, or the pharmaceutical composition of the present disclosure.

In a further aspect, the present disclosure provides a method of treating diseases, disorders or conditions in a subject in need thereof, comprising administering to the subject a therapeutically effective amount of the compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, or the pharmaceutical composition of the present disclosure. In some embodiments, the diseases, disorders or conditions are BCL-2 or BCL-2/BCL-XL associated diseases, disorders or conditions.

In a further aspect, the present disclosure provides the compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, or the pharmaceutical composition of the present disclosure for use in the treatment of diseases, disorders or conditions (e.g. BCL-2 or BCL-2/BCL-XL associated diseases, disorders or conditions).

In a further aspect, the present disclosure provides use of the compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, or the pharmaceutical composition of the present disclosure, in the manufacture of a medicament for the treatment of diseases, disorders or conditions (e.g. BCL-2 or BCL-2/BCL-XL associated diseases, disorders or conditions).

In a further aspect, the present disclosure provides a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, or the pharmaceutical composition of the present disclosure for use in the treatment of diseases, disorders or conditions (e.g. BCL-2 or BCL-2/BCL-XL associated diseases, disorders or conditions), wherein the compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, or the pharmaceutical composition of the present disclosure is administered simultaneously, separately or sequentially with a second therapy.

In a further aspect, the present disclosure provides a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, or the pharmaceutical composition of the present disclosure, administered simultaneously, separately or sequentially with at least one additional anti-tumor agent.

### DETAILED DESCRIPTION OF THE DISCLOSURE

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination. It must be noted that, as used in the specification and the appended claims, the singular forms “a,” “an,” and “the” include plural forms of the same unless the context clearly dictates otherwise. Thus, for example, reference to “a compound” includes a plurality of compounds.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, 2^{nd} Edition, University Science Books, Sausalito, 2006; Smith and March March's Advanced Organic Chemistry, 6^{th} Edition, John Wiley & Sons, Inc., New York, 2007; Larock, Comprehensive Organic Transformations, 3^{r}d Edition, VCH Publishers, Inc., New York, 2018; Carruthers, Some Modern Methods of Organic Synthesis, 4^{th} Edition, Cambridge University Press, Cambridge, 2004; the entire contents of each of which are incorporated herein by reference.

At various places in the present disclosure, linking substituents are described. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, the term “Cᵢ₋ⱼ” indicates a range of the carbon atoms numbers, wherein i and j are integers and the range of the carbon atoms numbers includes the endpoints (i.e. i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to an alkyl having i to j carbon atoms. In some embodiments, alkyl groups contain 1 to 10 carbon atoms. In some embodiments, alkyl groups contain 1 to 9 carbon atoms. In some embodiments, alkyl groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

The alkyl groups can be further substituted by substituents which independently replace one or more hydrogen atoms on one or more carbons of the alkyl groups. Examples of such substituents can include, but are not limited to, acyl, alkyl, alkenyl, alkynyl, halogen, hydroxyl, alkoxyl, haloalkyl, haloalkoxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonate, sulfamoyl, sulfonamido, nitro, trifluoromethyl, cyano, nitro, azido, heterocyclyl, alkylaryl, or an aromatic or heteroaromatic moiety. Alkenyl, alkynyl, cycloalkyl, heterocyclyl, aryl and heteroaryl groups as described below may also be similarly substituted.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. Examples of alkenyl group include, but are not limited to, ethylenyl (or vinyl), propenyl, butenyl, pentenyl, 1-methyl-2 buten-1-yl, 5-hexenyl, and the like.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkynyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like.

As used herein, the term “alkoxyl”, whether as part of another term or used independently, refers to an alkyl group, as previously defined, attached to the parent molecule through an oxygen atom. The term “Cᵢ₋ⱼ alkoxyl” means that the alkyl moiety of the alkoxy group has i to j carbon atoms. In some embodiments, alkoxy groups contain 1 to 10 carbon atoms. In some embodiments, alkoxy groups contain 1 to 9 carbon atoms. In some embodiments, alkoxy groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₆ alkoxyl” include, but are not limited to, methoxy, ethoxy, propoxy (e.g. n-propoxy and isopropoxy), t-butoxy, neopentoxy, n-hexoxy, and the like.

As used herein, the term “alkylalkoxyl”, whether as part of another term or used independently, refers to an alkyl moiety substituted with one or more alkoxyl moiety. The “alkylalkoxyl” can be bonded to the parent molecular structure through the alkyl group or the alkoxyl group.

As used herein, the term “alkylcycloalkyl”, whether as part of another term or used independently, refers to an alkyl moiety substituted with one or more cycloalkyl moiety. The “alkylcycloalkyl” can be bonded to the parent molecular structure through the alkyl group or the cycloalkyl group.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused or bridged. Examples of polycyclic aryl include, but are not limited to, benzofuranyl, indanyl, phthalimidyl, naphthimidyl, phenanthridinyl, or tetrahydronaphthyl, and the like. Aryl groups can be substituted at one or more ring positions with substituents as described above.

As used herein, the term “cycloalkyl”, whether as part of another term or used independently, refer to a monovalent non-aromatic, saturated or partially unsaturated monocyclic and polycyclic ring system, in which all the ring atoms are carbon and which contains at least three ring forming carbon atoms. In some embodiments, the cycloalkyl may contain 3 to 12 ring forming carbon atoms, 3 to 11 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 3 to 4 ring forming carbon atoms, 4 to 12 ring forming carbon atoms, 4 to 11 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated. Cycloalkyl groups may be substituted. In some embodiments, the cycloalkyl group may be a saturated cyclic alkyl group. In some embodiments, the cycloalkyl group may be a partially unsaturated cyclic alkyl group that contains at least one double bond or triple bond in its ring system.

In some embodiments, the cycloalkyl group may be monocyclic or polycyclic. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl.

In some embodiments, the cycloalkyl group may be saturated or partially unsaturated polycyclic (e.g., bicyclic and tricyclic) carbocyclic ring system, which can be arranged as a fused-, spiro- or bridged-ring system. As used herein, the term “fused-ring” refers to a ring system having two rings sharing two adjacent atoms, the term “spiro-ring” refers to a ring systems having two rings connected through one single common atom, and the term “bridged-ring” refers to a ring system with two rings sharing three or more atoms. Examples of fused carbocyclyl include, but are not limited to, naphthyl, benzopyrenyl, anthracenyl, acenaphthenyl, fluorenyl and the like. Examples of spiro carbocyclyl include, but are not limited to, spiro[5.5]undecanyl, spiro-pentadienyl, spiro[3.6]-decanyl, and the like. Examples of bridged carbocyclyl include, but are not limited to bicyclo[1,1,1]pentenyl, bicyclo[2,2,1]heptenyl, bicyclo[2.2.1]heptanyl, bicyclo[2.2.2]octanyl, bicyclo[3.3.1]nonanyl, bicyclo[3.3.3]undecanyl, and the like.

As used herein, the term “cyano” refers to —CN.

As used herein, the term “halogen” refers to an atom selected from fluorine (or fluoro), chlorine (or chloro), bromine (or bromo) and iodine (or iodo).

As used herein, the term “haloalkyl”, whether as part of another term or used independently, refers to an alkyl group having one or more halogen substituents. Examples of haloalkyl group include, but are not limited to, trifluoromethyl (—CF₃), pentafluoroethyl (—C₂F₅), difluoromethyl (—CHF₂), trichloromethyl (—CCl₃), dichloromethyl (—CHCl₂), pentachloroethyl (—C₂Cl₅), and the like.

As used herein, the term “haloalkoxyl”, whether as part of another term or used independently, refers to an alkoxyl group having one or more halogen substituents. As a result, the term “halo-Cᵢ₋ⱼ alkoxyl”, whether as part of another term or used independently, refers to a Cᵢ₋ⱼ alkoxyl group having one or more halogen substituents. Examples of haloalkoxyl include, but are not limited to, —O—CF₃, —O—C₂F₅, —O—CHF₂, —O—CCl₃, —O—CHCl₂, —O—C₂Cl₅, and the like.

As used herein, the term “heteroatom” refers to nitrogen (N), oxygen (O), sulfur (S), and includes any oxidized form of nitrogen or sulfur, and any quaternized form of a basic nitrogen (including N-oxides).

As used herein, the term “heteroalkyl”, “heteroalkenyl”, or “heteroalkynyl”, whether as part of another term or used independently, refers to an alkyl, alkenyl, or alkynyl group containing one or more heteroatoms. As a result, the term “hetero-Cᵢ₋ⱼ alkyl”, “hetero-Cᵢ₋ⱼ alkenyl”, or “hetero-Cᵢ₋ⱼ alkynyl”, whether as part of another term or used independently, refers to a Cᵢ₋ⱼ alkyl, Cᵢ₋ⱼ alkenyl, or Cᵢ₋ⱼ alkynyl containing one or more heteroatoms. For example, the term “hetero-C₁₋₆ alkyl”, whether as part of another term or used independently, refers to a C₁₋₆ alkyl containing one or more heteroatoms.

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, heteroaryl, cycloaliphatic, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen or sulfur, and any quaternized form of a basic nitrogen. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

Heterocyclyl group may be monocyclic. Examples of monocyclic heterocyclyl include, but are not limited to oxetanyl, 1,1-dioxothietanylpyrrolidyl, tetrahydrofuryl, tetrahydropyranyl, tetrahydrothienyl, azetidinyl, pyrrolyl, furanyl, thienyl, pyrazolyl, imidazolyl, triazolyl, oxazolyl, thiazolyl, piperidyl, piperazinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like.

Heterocyclyl group may be polycyclic, including the fused-, spiro- and bridged-ring systems. The fused heterocyclyl group includes radicals wherein the heterocyclyl radicals are fused with a saturated, partially unsaturated, or fully unsaturated (i.e., aromatic) carbocyclic or heterocyclic ring. Examples of fused heterocyclyl include, but are not limited to, phenyl fused-ring or pyridinyl fused-ring, such as quinolinyl, isoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, imidazo[1,2-a]pyridinyl, [1,2,4]triazolo[4,3-a]pyridinyl, [1,2,3]triazolo[4,3-a]pyridinyl groups, and the like. Examples of spiro heterocyclyl include, but are not limited to, spiropyranyl, spirooxazinyl, 5-aza-spiro[2.4]heptanyl, 6-aza-spiro[2.5]octanyl, 6-aza-spiro[3.4]octanyl, 2-oxa-6-aza-spiro[3.3]heptanyl, 2-oxa-6-aza-spiro[3.4]octanyl, 6-aza-spiro[3.5]nonanyl, 7-aza-spiro[3.5]nonanyl, 1-oxa-7-aza-spiro[3.5]nonanyl and the like. Examples of bridged heterocyclyl include, but are not limited to, 3-aza-bicyclo[3.1.0]hexanyl, 8-aza-bicyclo[3.2.1]octanyl, 1-aza-bicyclo[2.2.2]octanyl, 2-aza-bicyclo[2.2.1]heptanyl, 1,4-diazabicyclo[2.2.2]octanyl, and the like.

As used herein, the term “hydroxyl” refers to —OH.

As used herein, the term “sulfhydryl” refers to —SH.

As used herein, the term “sulfonyl” refers to —SO₂R′, wherein R′ is selected from hydrogen, alkyl, alkenyl, alkynyl, haloalkyl, cycloalkyl, heterocyclyl, aryl, or heteroaryl.

As used herein, the term “-Boc” refers to t-butoxyl carbonyl.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

### Compounds

The present disclosure provides novel compounds or tautomers, stereoisomers, or pharmaceutically acceptable salts thereof, synthetic methods for making the compounds, pharmaceutical compositions containing them and various uses of the disclosed compounds.

In one aspect, the present disclosure provides a compound of Formula I.

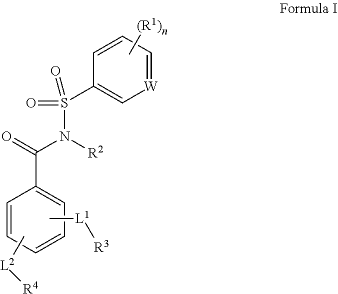

or a tautomer, a stereoisomer, or a pharmaceutically acceptable salt thereof, wherein
W is N or C(R¹);
n is 0, 1, 2 or 3;
each R¹ is independently selected from the group consisting of hydrogen, halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, and —NH-L³-R^{a}, wherein,
L³ is absent or selected from alkyl, alkenyl, or alkynyl, each of which is optionally substituted with one or more R^{b};
R^{a} is selected from the group consisting of cycloalkyl, heterocyclyl, aryl and heteroaryl, wherein each of the cycloalkyl, heterocyclyl, aryl and heteroaryl is optionally substituted with one or more R^{c};
R² is selected from the group consisting of hydrogen, alkyl, alkenyl, alkynyl, heteroalkyl, heteroalkenyl, heteroalkynyl, haloalkyl, and alkylalkoxyl;
L¹ is absent, O, S, or N;
R³ is absent, cycloalkyl, heterocyclyl, aryl or heteroaryl, wherein each of cycloalkyl, heterocyclyl, aryl or heteroaryl is optionally substituted with one or more R^{d};
L² is selected from the group consisting of C₁₋₆ alkyl, C₁₋₆ alkenyl, C₁₋₆ alkynyl, halo-C₁₋₆ alkyl, hetero-C₁₋₆ alkenyl, hetero-C₁₋₆ alkynyl, cycloalkyl, heterocyclyl, aryl and heteroaryl, each of which is optionally substituted with one or more R^{e};
R⁴ is

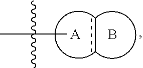

wherein
Ring A is selected from the group consisting of cycloalkyl, heterocyclyl, aryl, and heteroaryl, each of which is optionally substituted with one or more R^{f};
Ring B is selected from the group consisting of cycloalkyl, heterocyclyl, aryl, and heteroaryl, each of which is optionally substituted with one or more R^{g};
- - - is a bond via which Ring A is fused to Ring B;
each R^{c} is independently selected from the group consisting of halogen, cyano, hydroxyl, sulfhydryl, —NH₂, —NO₂, alkyl, alkenyl, alkynyl, heteroalkyl, heteroalkenyl, heteroalkynyl, haloalkyl, alkoxyl, haloalkoxyl, cycloalkyl, heterocyclyl, aryl, heteroaryl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3};
R^{b}, R^{d} and R^{e} are each independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl;
each R^{f} is independently selected from the group consisting of oxo, halogen, cyano, hydroxyl, sulfhydryl, —NH₂, —NO₂, alkyl, alkenyl, alkynyl, heteroalkyl, heteroalkenyl, heteroalkynyl, haloalkyl, alkoxyl, haloalkoxyl, cycloalkyl, heterocyclyl, aryl, heteroaryl, and —S(O)₂—R^{a4};
each R^{g} is independently selected from the group consisting of oxo, halogen, cyano, hydroxyl, sulfhydryl, —NH₂, —NO₂, alkyl, alkenyl, alkynyl, heteroalkyl, heteroalkenyl, heteroalkynyl, haloalkyl, alkoxyl, haloalkoxyl, cycloalkyl, heterocyclyl, aryl, heteroaryl, —NH—C(O)—R^{a5}, —NH—S(O)₂—R^{a5}, —P(O)(R^{a5})₂, —S(O)₂—R^{a5} wherein each of alkyl, alkenyl, alkynyl, heteroalkyl, heteroalkenyl, heteroalkynyl, cycloalkyl, heterocyclyl, aryl, and heteroaryl is optionally substituted with one or more of a group selected from halogen, cyano, hydroxyl, sulfhydryl, —NH₂, —NO₂, alkyl, alkenyl, alkynyl, heteroalkyl, heteroalkenyl, or heteroalkynyl;
R^{a1}, R^{a2} and R^{a3} are each independently selected from the group consisting of hydrogen, hydroxyl, halogen, alkyl, haloalkyl, alkoxyl, cycloalkyl and -alkyl-NH₂;
R^{a4} and R^{a5} are each independently selected from the group consisting of alkyl, alkenyl, alkynyl, heteroalkyl, heteroalkenyl, heteroalkynyl, cycloalkyl, heterocyclyl, aryl and heteroaryl, wherein each of cycloalkyl, heterocyclyl, aryl and heteroaryl is optionally substituted with one or more of a group selected from halogen, cyano, hydroxyl, sulfhydryl, —NH₂, —NO₂, alkyl, alkenyl, alkynyl, heteroalkyl, heteroalkenyl, or heteroalkynyl.

In another aspect, the present disclosure provides a compound of Formula II.

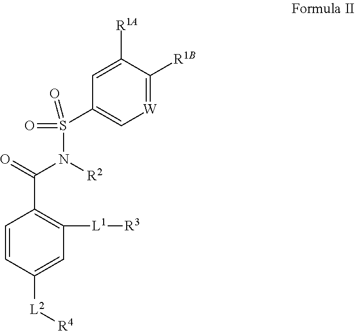

or a tautomer, a stereoisomer, or a pharmaceutically acceptable salt thereof, wherein
W is N or C(R¹);
R^{1A} is selected from the group consisting of hydrogen, halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, and haloalkoxyl;
R^{1B} is absent or —NH-L³-R^{a};
R¹, R², L¹, R³, L², R⁴, L³, R^{a} are each defined as supra.

In some embodiments, W is N. In some embodiments, W is C(R¹). In some embodiments, W is CH.

In some embodiments, n is 0. In some embodiments, n is 1. In some embodiments, n is 2. In some embodiments, n is 3.

In some embodiments, R^{1A} is —NO₂. In some embodiments, R^{1A} is —SO₂— alkyl. In some embodiments, R^{1A} is —SO₂-haloalkyl. In some embodiments, R^{1A} is —SO₂CF₃. In some embodiments, R^{1A} is —SO₂CHF₂. In some embodiments, R^{1A} is —SO₂CH₂F. In some embodiments, R^{1A} is —SO₂CH₃.

In some embodiments, R^{1B} is absent. In some embodiments, R^{1B} is —NH-L³-R^{a}.

In some embodiments, R^{1B} is —NH-L³-R^{a}, wherein L³ is absent.

In some embodiments, R^{1B} is —NH-L³-R^{a}, wherein L³ is alkyl optionally substituted with one or more R^{b}, and each R^{b} is independently selected from the group consisting of halogen, cyano, hydroxyl, sulfhydryl, —NH₂, —NO₂, —SO₂-alkyl, —SO₂— haloalkyl, alkyl, haloalkyl, alkoxyl, and haloalkoxyl.

In some embodiments, L³ is C₁₋₆ alkyl, C₁₋₅ alkyl, Cia alkyl, or C₁₋₃ alkyl, optionally substituted with 1, 2 or 3 R^{b}, and each R^{b} is independently selected from the group consisting of halogen, cyano, hydroxyl, sulfhydryl, —NH₂, —NO₂, —SO₂-alkyl, —SO₂-haloalkyl, alkyl, haloalkyl, alkoxyl, and haloalkoxyl.

In some embodiments, L³ is methyl, ethyl, propyl, butyl, pentyl, or hexyl, optionally substituted with 1 or 2 R^{b}, and each R^{b} is independently selected from the group consisting of halogen, cyano, hydroxyl, sulfhydryl, —NH₂, —NO₂, —SO₂—CF₃, and C₁₋₆ alkyl.

In some embodiments, L³ is methyl optionally substituted with a halogen. In some embodiments, L³ is —CH₂—. In some embodiments, L³ is —CH₂CH₂—. In some embodiments, L³ is propyl. In some embodiments, L³ is n-propyl (—CH₂CH₂CH₂—) or isopropyl (—CH(CH₃)CH₂—).

In some embodiments, R^{a} is selected from the group consisting of cycloalkyl, heterocyclyl, aryl and heteroaryl, wherein each of the cycloalkyl, heterocyclyl, aryl and heteroaryl is optionally substituted with one or more R^{c}. In some embodiments, each R^{c} is independently selected from the group consisting of halogen, cyano, hydroxyl, sulfhydryl, —NH₂, —NO₂, alkyl, alkenyl, alkynyl, heteroalkyl, heteroalkenyl, heteroalkynyl, haloalkyl, alkoxyl, haloalkoxyl, cycloalkyl, heterocyclyl, aryl, heteroaryl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3}, wherein R^{a1}, R^{a2} and R^{a3} are each independently selected from the group consisting of hydrogen, hydroxyl, halogen, alkyl, haloalkyl, alkoxyl, cycloalkyl and -alkyl-NH₂.

In some embodiments, R^{a} is cycloalkyl, heterocyclyl or heteroaryl, wherein each of the cycloalkyl, heterocyclyl and heteroaryl is optionally substituted with one or more R^{c}, and each R^{c} is independently selected from the group consisting of hydroxyl, alkyl, haloalkyl, heterocyclyl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3}, wherein R^{a1}, R^{a2} and R^{a3} are each independently selected from the group consisting of hydrogen, hydroxyl, halogen, alkyl, haloalkyl, alkoxyl, cycloalkyl and -alkyl-NH₂.

In some embodiments, R^{a} is heterocyclyl, which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, -alkyl-cycloalkyl, -alkyl-OH, -alkyl-COOH, -alkyl-C(O)-alkoxyl, —S(O)₂-alkyl, —S(O)₂-cycloalkyl, —C(O)-alkyl, and —C(O)-alkyl-NH₂.

In some embodiments, R^{a} is 3- to 12-membered heterocyclyl, 3- to 11-membered heterocyclyl, 3- to 10-membered heterocyclyl, 3- to 9-membered heterocyclyl, 3- to 8-membered heterocyclyl, 3- to 7-membered heterocyclyl, 3- to 6-membered heterocyclyl, 3- to 5-membered heterocyclyl, or 3- to 4-membered heterocyclyl, which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, -alkyl-cycloalkyl, -alkyl-OH, -alkyl-COOH, -alkyl-C(O)-alkoxyl, —S(O)₂-alkyl, —S(O)₂-cycloalkyl, —C(O)-alkyl, and —C(O)-alkyl-NH₂.

In some embodiments, R^{a} is 3- to 12-membered, 3- to 11-membered, 3- to 10-membered, 3- to 9-membered, 3- to 8-membered, 3- to 7-membered, 3- to 6-membered, 3- to 5-membered heterocyclyl, or 3- to 4-membered heterocyclyl containing one or more (e.g. 1, 2, 3, 4 or more) heteroatoms (e.g. O, N, S), which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, -alkyl-cycloalkyl, -alkyl-OH, -alkyl-COOH, -alkyl-C(O)-alkoxyl, —S(O)₂-alkyl, —S(O)₂-cycloalkyl, —C(O)-alkyl, and —C(O)-alkyl-NH₂.

In some embodiments, R^{a} is a monocyclic heterocyclyl, which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, and —C(O)-alkyl.

In some embodiments, R^{a} is a 3- to 12-membered, 3- to 11-membered, 3- to 10-membered, 3- to 9-membered, 3- to 8-membered, 3- to 7-membered, 3- to 6-membered, 3- to 5-membered, or 3- to 4-membered monocyclic heterocyclyl, which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, and —C(O)-alkyl.

In some embodiments, R^{a} is a 3- to 12-membered, 3- to 11-membered, 3- to 10-membered, 3- to 9-membered, 3- to 8-membered, 3- to 7-membered, 3- to 6-membered, 3- to 5-membered, or 3- to 4-membered monocyclic heterocyclyl containing one or more (e.g. 1, 2, 3, 4 or more) heteroatoms (e.g. O, N, S), which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, and —C(O)-alkyl.

In some embodiments, R^{a} is selected from the group consisting of

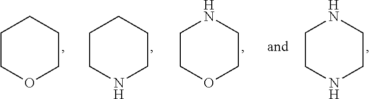

each of which is optionally substituted with one or more R^{c}, and each R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, and —C(O)-alkyl.

In some embodiments, R^{a} is selected from the group consisting of

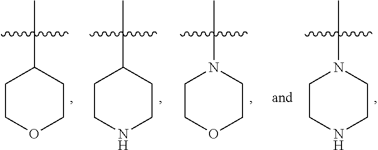

each of which is optionally substituted with one or more R^{c}, and each R^{c} is independently selected from the group consisting of hydroxyl, alkyl, heterocyclyl, and —C(O)-alkyl.

In some embodiments, R^{a} is selected from the group consisting of

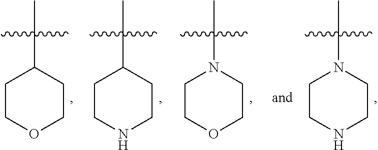

each of which is optionally substituted with 1, 2, or 3 R^{c}, and each R^{c} is independently selected from the group consisting of hydroxyl, C₁₋₆ alkyl (e.g. methyl, ethyl, propyl, butyl, pentyl, hexyl, heptyl), 3- to 12-membered heterocyclyl, and —C(O)—C₁₋₆ alkyl.

In some embodiments, R^{a} is selected from the group consisting of

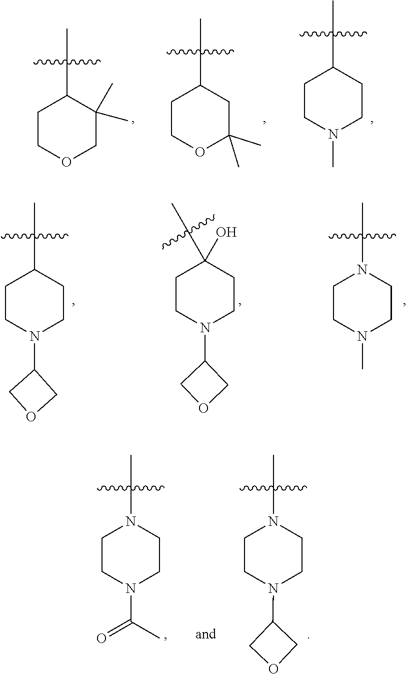

In some embodiments, R^{a} is a polycyclic (such as bicyclic or tricyclic) heterocyclyl, which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, -alkyl-cycloalkyl, -alkyl-OH, -alkyl-COOH, -alkyl-C(O)-alkoxyl, —S(O)₂-alkyl, —S(O)₂-cycloalkyl, —C(O)-alkyl, and —C(O)-alkyl-NH₂.

In some embodiments, R^{a} is a 5- to 15-membered, 5- to 14-membered, 5- to 13-membered, 5- to 12-membered, 5- to 11-membered, 5- to 10-membered, 5- to 9-membered, 5- to 8-membered, 5- to 7-membered, or 5- to 6-membered polycyclic (such as bicyclic or tricyclic) heterocyclyl, which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, -alkyl-cycloalkyl, -alkyl-OH, -alkyl-COOH, -alkyl-C(O)-alkoxyl, —S(O)₂-alkyl, —S(O)₂-cycloalkyl, —C(O)-alkyl, and —C(O)-alkyl-NH₂.

In some embodiments, R^{a} is a 5- to 15-membered, 5- to 14-membered, 5- to 13-membered, 5- to 12-membered, 5- to 11-membered, 5- to 10-membered, 5- to 9-membered, 5- to 8-membered, 5- to 7-membered, or 5- to 6-membered polycyclic (such as bicyclic or tricyclic) heterocyclyl containing one or more (e.g. 1, 2, 3, 4 or more) heteroatoms (e.g. O, N, S), which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, -alkyl-cycloalkyl, -alkyl-OH, -alkyl-COOH, -alkyl-C(O)-alkoxyl, —S(O)₂-alkyl, —S(O)₂-cycloalkyl, —C(O)-alkyl, and —C(O)-alkyl-NH₂.

In some embodiments, R^{a} is a polycyclic (such as bicyclic or tricyclic) cycloalkyl, which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, -alkyl-cycloalkyl, -alkyl-OH, -alkyl-COOH, -alkyl-C(O)-alkoxyl, —S(O)₂-alkyl, —S(O)₂-cycloalkyl, —C(O)-alkyl, and —C(O)-alkyl-NH₂.

In some embodiments, R^{a} is a 5- to 15-membered, 5- to 14-membered, 5- to 13-membered, 5- to 12-membered, 5- to 11-membered, 5- to 10-membered, 5- to 9-membered, 5- to 8-membered, 5- to 7-membered, or 5- to 6-membered polycyclic (such as bicyclic or tricyclic) cycloalkyl, which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, -alkyl-cycloalkyl, -alkyl-OH, -alkyl-COOH, -alkyl-C(O)-alkoxyl, —S(O)₂-alkyl, —S(O)₂-cycloalkyl, —C(O)-alkyl, and —C(O)-alkyl-NH₂.

In some embodiments, R^{a} is a spiro-ring system, which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, -alkyl-cycloalkyl, -alkyl-OH, -alkyl-COOH, -alkyl-C(O)-alkoxyl, —S(O)₂-alkyl, —S(O)₂-cycloalkyl, —C(O)-alkyl, and —C(O)— alkyl-NH₂.

In some embodiments, R^{a} is a spiro-ring system containing one or more (e.g. 1, 2, 3, 4 or more) heteroatoms (e.g. O, N, S), which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, -alkyl-cycloalkyl, -alkyl-OH, -alkyl-COOH, -alkyl-C(O)-alkoxyl, —S(O)₂-alkyl, —S(O)₂-cycloalkyl, —C(O)-alkyl, and —C(O)-alkyl-NH₂.

In some embodiments, R^{a} is a spiro-ring system, which is optionally substituted with one or more R^{c}, and R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, -alkyl-cycloalkyl, -alkyl-OH, -alkyl-COOH, -alkyl-C(O)-alkoxyl, —S(O)₂-alkyl, —S(O)₂-cycloalkyl, —C(O)-alkyl, and —C(O)— alkyl-NH₂, and in the spiro-ring system, the number of members of one ring linked to L³ is equal or less than that of the other ring. For example, in the spiro-ring system, the ring linked to L³ is a 4- to 10-membered ring, and the other ring is a 4- to 11-membered ring, provided that the number of members of one ring linked to L³ is equal or less than that of the other ring. In some embodiments, in the spiro-ring system, the ring linked to L³ is a 4-membered ring, and the other ring is a 6-membered ring.

In some embodiments, R^{a} is selected from the group consisting of

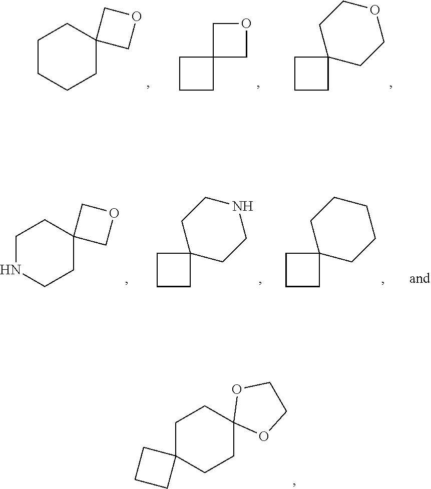

each of which is optionally substituted with one or more R, and each R^{c} is independently selected from the group consisting of halogen, cyano, hydroxyl, sulfhydryl, —NH₂, —NO₂, alkyl, heteroalkyl, haloalkyl, alkoxyl, haloalkoxyl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3}, and wherein R^{a1}, R^{a2} and R^{a3} are each independently selected from the group consisting of hydrogen, hydroxyl, halogen, alkyl, haloalkyl, alkoxyl, cycloalkyl and -alkyl-NH₂.

In some embodiments, R^{a} is selected from the group consisting of

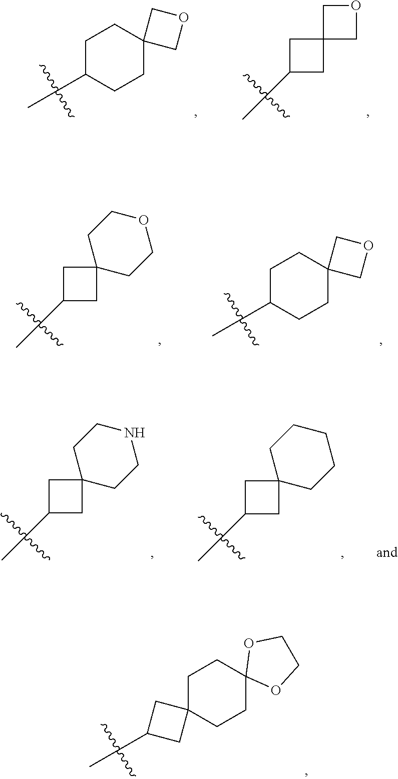

each of which is optionally substituted with one or more R^{c}, and each R^{c} is independently selected from the group consisting of alkyl, haloalkyl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3}, and wherein R^{a1}, R^{a2} and R^{a3} are each independently selected from the group consisting of hydrogen, hydroxyl, halogen, alkyl, haloalkyl, alkoxyl, cycloalkyl and -alkyl-NH₂.

In some embodiments, R^{a} is selected from the group consisting of

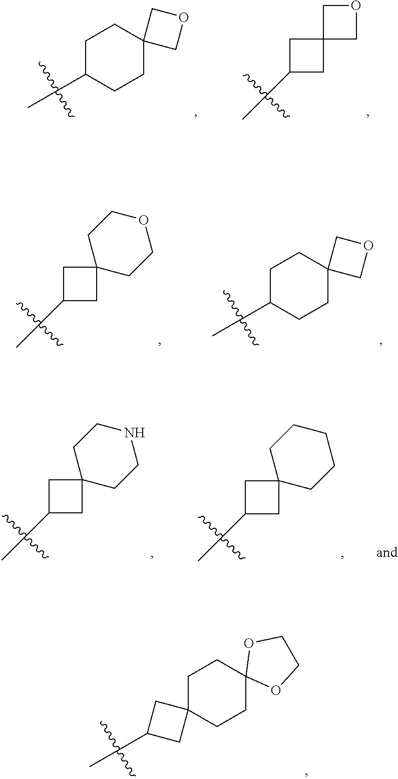

each of which is optionally substituted with 1, 2, or 3 R^{c}, and each R^{c} is independently selected from the group consisting of C₁₋₆ alkyl, C₁₋₆ alkyl substituted with 1, 2 or 3 halogens (e.g. fluoro), —C(O)—C₁₋₆ alkyl, —S(O)₂—C₁₋₆ alkyl, —S(O)₂—C₃₋₆ cycloalkyl, —C₁₋₆ alkyl-C₃₋₆ cycloalkyl, —C₁₋₆ alkyl-NHR^{a3}, —C₁₋₆ alkyl-NHC(O)R^{a3}, —C₁₋₆ alkyl-OH, —C₁₋₆ alkyl-C(O)—O—C₁₋₆ alkyl, or —C₁₋₆ alkyl-COOH, and wherein R^{a3} is hydrogen or —O—C₁₋₆ alkyl. In some embodiments, R^{a} is optionally substituted with one or more of methyl, ethyl, propyl, -Boc, —CH₂CH₂—NH-Boc, —CH₂CH₂NH₂, —CH₂CH₂NHC(O)CH₃, —C(O)CH₃, —S(O)₂CH₃, —CH₂CH₂—OH, —(CH₂)₁₋₂C(O)O—CH₂CH₃, —(CH₂)₁₋₂COOH, —C(O)CH(CH₃)₂, —C(O)C(NH₂)(CHCH₃CH₃), —S(O)₂-cyclopropyl, —S(O)₂—CH(CH₃)₂, —CH₂-cyclopropyl, hydroxyl, or halogen (e.g. F, Cl, Br, or I). In some embodiments, R^{a} is optionally substituted with one R^{c}, and R^{c} is C₁₋₆ alkyl, C₁₋₅ alkyl, C₁₋₄ alkyl, or C₁₋₃ alkyl substituted with 1, 2 or 3 halogens. In some embodiments, R^{a} is optionally substituted with one R^{c}, and R^{c} is C₁₋₆ alkyl, C₁₋₅ alkyl, C₁₋₄ alkyl, or C₁₋₃ alkyl substituted with 1, 2 or 3 fluoro. In some embodiments, R^{a} is optionally substituted with —CH₂CH₂F, —CH₂CHF₂, or —CH₂CF₃.

In some embodiments, R^{a} is selected from the group consisting of

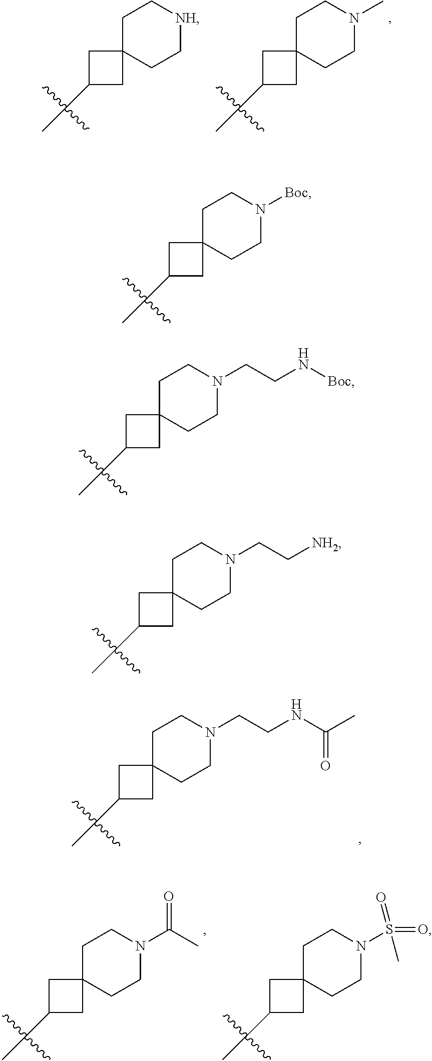

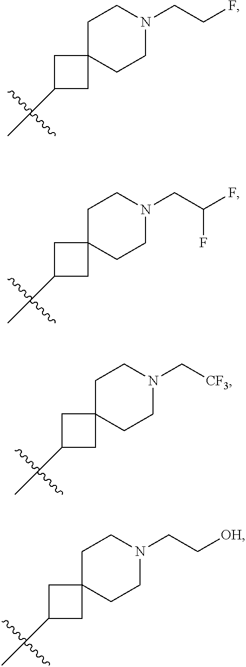 [SMILES: *C1CC2(CCN(CCF)CC2)C1 ; *C1CC2(CCN(CC(F)F)CC2)C1 ; *C1CC2(CCN(CC(F)(F)F)CC2)C1 ; *C1CC2(CCN(CCO)CC2)C1]

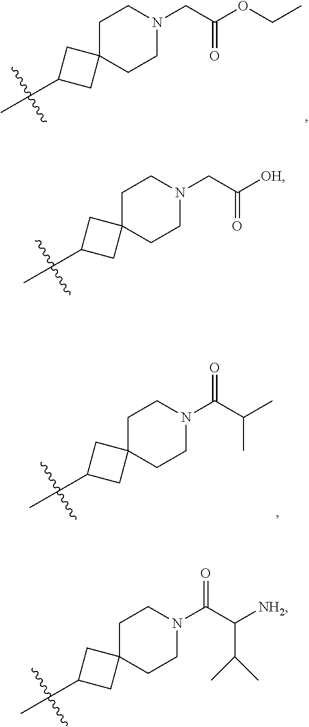 [SMILES: *C1CC2(CCN(CC(=O)OCC)CC2)C1 ; *C1CC2(CCN(CC(=O)O)CC2)C1 ; *C1CC2(CCN(C(=O)C(C)C)CC2)C1 ; *C1CC2(CCN(C(=O)C(N)C(C)C)CC2)C1]

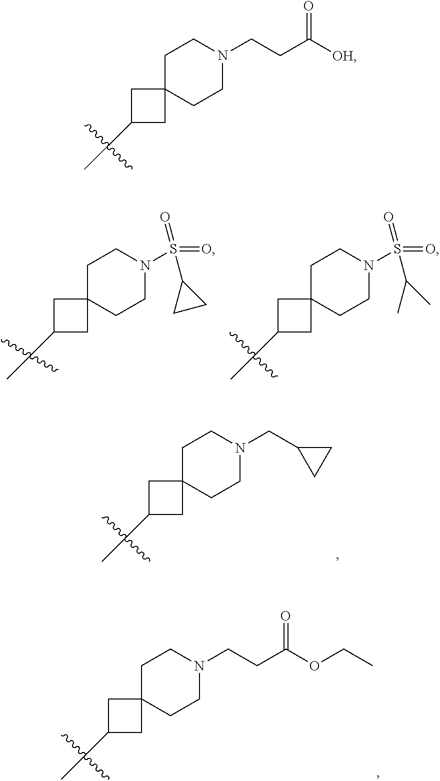 [SMILES: *C1CC2(CCN(CCC(=O)O)CC2)C1 ; *C1CC2(CCN(S(=O)(=O)C3CC3)CC2)C1 ; *C1CC2(CCN(S(=O)(=O)C(C)C)CC2)C1 ; *C1CC2(CCN(CC3CC3)CC2)C1 ; *C1CC2(CCN(CCC(=O)OCC)CC2)C1]

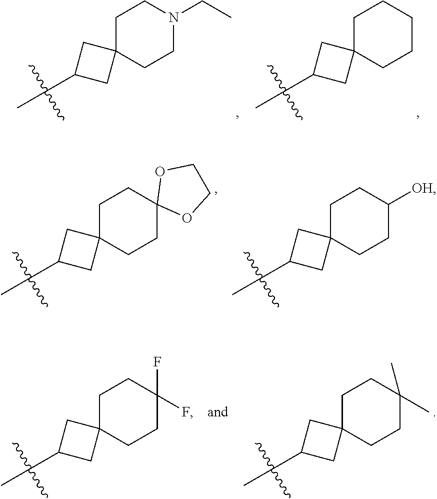 [SMILES: *C1CC2(CCN(CC)CC2)C1 ; *C1CC2(CCCCC2)C1 ; *C1CC2(CCC3(CC2)OCCO3)C1 ; *C1CC2(CCC(O)CC2)C1 ; *C1CC2(CCC(F)(F)CC2)C1 ; *C1CC2(CCC(C)(C)CC2)C1]

In some embodiments, R^{a} is a bridged-ring system, which is optionally substituted with one or more R^{c}, and each R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, and —C(O)-alkyl.

In some embodiments, R^{a} is a 5- to 12-membered, 5- to 11-membered, 5- to 10-membered, 5- to 9-membered, 5- to 8-membered, 5- to 7-membered, or 5- to 6-membered bridged-ring system, which is optionally substituted with one or more R^{c}, and each R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, and —C(O)-alkyl.

In some embodiments, R^{a} is a 5- to 12-membered, 5- to 11-membered, 5- to 10-membered, 5- to 9-membered, 5- to 8-membered, 5- to 7-membered, or 5- to 6-membered bridged-ring system containing one or more (e.g. 1, 2, 3, 4 or more) heteroatoms (e.g. O, N, S), which is optionally substituted with one or more R^{c}, and each R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, and —C(O)-alkyl.

In some embodiments, R^{a} is selected from the group consisting of

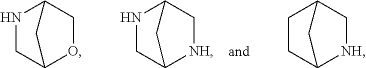 [SMILES: C1OC2CNC1C2 ; C1NC2CNC1C2 ; "and" ; C1CC2CC1CN2]

each of which is optionally substituted with one or more R, and each R^{c} is independently selected from the group consisting of halogen, cyano, hydroxyl, sulfhydryl, —NH₂, —NO₂, alkyl, heteroalkyl, haloalkyl, alkoxyl, haloalkoxyl, and —C(O)—R^{a1}, wherein R^{a1} is selected from the group consisting of hydrogen, hydroxyl, halogen, alkyl, haloalkyl, and alkoxyl.

In some embodiments, R^{a} is selected from the group consisting of:

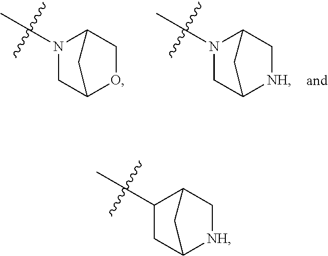 [SMILES: *N1CC2CC1CO2 ; *N1CC2CC1CN2 ; "and" ; *C1CC2CC1CN2]

each of which is optionally substituted with 1, 2, or 3 R^{c}, and each R^{c} is independently selected from C₁₋₆ alkyl or —C(O)—R^{a1}, wherein R^{a} is selected from the group consisting of hydrogen, hydroxyl, halogen, C₁₋₆ alkyl, halo-C₁₋₆ alkyl, and —O—C₁₋₆ alkyl. In some embodiments, R^{a} is optionally substituted with C₁₋₆ alkyl or -Boc.

In some embodiments, R^{a} is selected from the group consisting of:

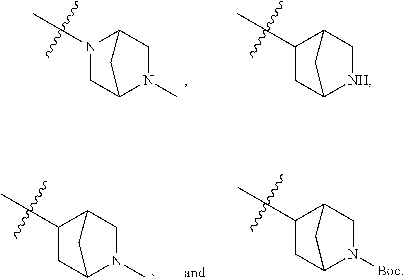

In some embodiments, R^{a} is a fused-ring system, which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, haloalkoxyl, cycloalkyl, heterocyclyl, and —C(O)-alkyl.

In some embodiments, R^{a} is a 5- to 12-membered, 5- to 11-membered, 5- to 10-membered, 5- to 9-membered, 5- to 8-membered, 5- to 7-membered, or 5- to 6-membered fused-ring system, which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, haloalkoxyl, cycloalkyl, heterocyclyl, and —C(O)-alkyl.

In some embodiments, R^{a} is a 5- to 12-membered, 5- to 11-membered, 5- to 10-membered, 5- to 9-membered, 5- to 8-membered, 5- to 7-membered, or 5- to 6-membered fused-ring system containing one or more (e.g. 1, 2, 3, 4 or more) heteroatoms (e.g. O, N, S), which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, haloalkoxyl, cycloalkyl, heterocyclyl, and —C(O)-alkyl.

In some embodiments, R^{a} is

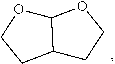

which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, and haloalkoxyl.

In some embodiments, R^{a} is

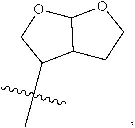 [SMILES: *C1COC2OCCC12]

which is optionally substituted with 1, 2, or 3 R^{c}, and each R^{c} is independently selected from the group consisting of halogen, cyano, hydroxyl, sulfhydryl, —NH₂, —NO₂, alkyl, heteroalkyl, haloalkyl, alkoxyl, and haloalkoxyl.

In some embodiments, R^{a} is

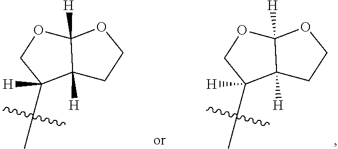 [SMILES: *[C@@H]1CO[C@@H]2OCC[C@@H]21 ; *[C@H]1CO[C@H]2OCC[C@H]21]

each of which is optionally substituted with 1, 2, or 3 R^{c}, and each R^{c} is independently selected from the group consisting of halogen, cyano, hydroxyl, sulfhydryl, —NH₂, —NO₂, C₁₋₆ alkyl, hetero-C₁₋₆ alkyl, halo-C₁₋₆ alkyl, —O—C₁₋₆ alkyl, and —O—C₁₋₆ haloalkyl.

In some embodiments, R^{a} is a heteroaryl containing one or more heteroatoms independently selected from O, S, or N atom, which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, and haloalkoxyl.

In some embodiments, R^{a} is a heteroaryl containing 1, 2, or 3 heteroatoms independently selected from O, S, or N atom, which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, and haloalkoxyl.

In some embodiments, R^{a} is a 5- to 12-membered, 5- to 11-membered, 5- to 10-membered, 5- to 9-membered, 5- to 8-membered, 5- to 7-membered, or 5- to 6-membered heteroaryl containing 1, 2 or 3 heteroatoms independently selected from 0, S, or N atom, which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, and haloalkoxyl.

In some embodiments, R^{a} is

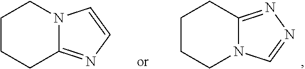 [SMILES: c1cn2c(n1)CCCC2 ; c1nnc2n1CCCC2]

each of which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, and haloalkoxyl.

In some embodiments, R^{a} is

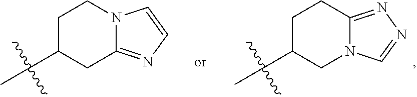 [SMILES: *C1CCn2ccnc2C1 ; *C1CCc2nncn2C1]

each of which is optionally substituted with one or more R^{c}, and each R^{c} is alkyl.

In some embodiments, R^{a} is

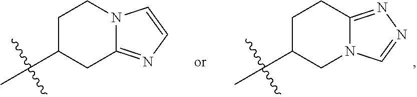 [SMILES: *C1CCn2ccnc2C1 ; *C1CCc2nncn2C1]

each of which is optionally substituted with 1, 2 or 3 R^{c}, and each R^{c} is independently selected from the group consisting of methyl, ethyl, propyl, butyl, pentyl, and hexyl.

In some embodiments, R^{a} is

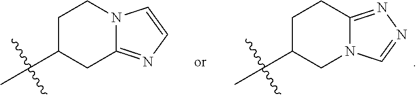 [SMILES: *C1CCn2ccnc2C1 ; *C1CCc2nncn2C1]

In some embodiments, R² is hydrogen.

In some embodiments, R² is alkyl or haloalkyl.

In some embodiments, R² is C₁₋₆ alkyl. In some embodiments, R² is selected from the group consisting of methyl, ethyl, propyl, butyl, pentyl, and hexyl.

In some embodiments, L¹ is absent. In some embodiments, L¹ is O. In some embodiments, L¹ is S. In some embodiments, L¹ is N.

In some embodiments, R³ is absent.

In some embodiments, R³ is cycloalkyl, which is optionally substituted with one or more R^{d}, and each R^{d} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R³ is 3- to 12-membered, 3- to 11-membered, 3- to 10-membered, 3- to 9-membered, 3- to 8-membered, 3- to 7-membered, 3- to 6-membered, 3- to 5-membered, or 3- to 4-membered cycloalkyl, which is optionally substituted with one or more R^{d}, and each R^{d} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R³ is heterocyclyl, which is optionally substituted with one or more R^{d}, and each R^{d} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R³ is heterocyclyl containing one or more (e.g. 1, 2, 3, 4, 5 or more) heteroatoms (e.g. O, N, S), which is optionally substituted with one or more R^{d}, and each R^{d} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R³ is 3- to 12-membered, 3- to 11-membered, 3- to 10-membered, 3- to 9-membered, 3- to 8-membered, 3- to 7-membered, 3- to 6-membered, 3- to 5-membered, or 3- to 4-membered heterocyclyl, which is optionally substituted with one or more R^{d}, and each R^{d} is independently selected from the group consisting of halogen, cyano, hydroxyl, sulfhydryl, —NH₂, —NO₂, —SO₂-alkyl, —SO₂— haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R³ is 3- to 12-membered, 3- to 11-membered, 3- to 10-membered, 3- to 9-membered, 3- to 8-membered, 3- to 7-membered, 3- to 6-membered, 3- to 5-membered, or 3- to 4-membered heterocyclyl containing one or more (e.g. 1, 2, 3, 4, 5 or more) heteroatoms (e.g. O, N, S), which is optionally substituted with one or more R^{d}, and each R^{d} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R³ is aryl (e.g. phenyl, biphenyl, naphthyl, anthracyl and the like), which is optionally substituted with one or more R^{d}, and each R^{d} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R³ is 3- to 12-membered, 3- to 11-membered, 3- to 10-membered, 3- to 9-membered, 3- to 8-membered, 3- to 7-membered, 3- to 6-membered, 3- to 5-membered, or 3- to 4-membered aryl, which is optionally substituted with one or more R^{d}, and each R^{d} is independently selected from the group consisting of halogen, cyano, hydroxyl, sulfhydryl, —NH₂, —NO₂, —SO₂-alkyl, —SO₂— haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R³ is heteroaryl, which is optionally substituted with one or more R^{d}, and each R^{d} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R³ is heteroaryl containing one or more (e.g. 1, 2, 3, 4, 5 or more) heteroatoms (e.g. O, N, S), which is optionally substituted with one or more R^{d}, and each R^{d} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R³ is 3- to 12-membered, 3- to 11-membered, 3- to 10-membered, 3- to 9-membered, 3- to 8-membered, 3- to 7-membered, 3- to 6-membered, 3- to 5-membered, or 3- to 4-membered heteroaryl, which is optionally substituted with one or more R^{d}, and each R^{d} is independently selected from the group consisting of halogen, cyano, hydroxyl, sulfhydryl, —NH₂, —NO₂, —SO₂-alkyl, —SO₂— haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R³ is 3- to 12-membered, 3- to 11-membered, 3- to 10-membered, 3- to 9-membered, 3- to 8-membered, 3- to 7-membered, 3- to 6-membered, 3- to 5-membered, or 3- to 4-membered heteroaryl containing one or more (e.g. 1, 2, 3, 4, 5 or more) heteroatoms (e.g. O, N, S), which is optionally substituted with one or more R^{d}, and each R^{d} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R³ is a heteroaryl containing one or more (e.g. 1, 2, 3, 4, 5 or more) N atoms, which is optionally substituted with one or more R^{d}, and each R^{d} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R³ is

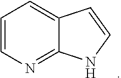

In some embodiments, R³ is

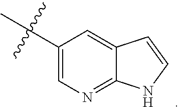 [SMILES: *c1cnc2[nH]ccc2c1]

In some embodiments, -L¹-R³ is absent. In some embodiments, -L¹-R³ is

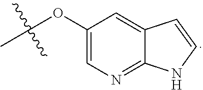 [SMILES: *Oc1cnc2[nH]ccc2c1]

In some embodiments, L² is a heterocyclyl optionally substituted with one or more R^{e}, and each R^{e} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, L² is a 3- to 12-membered, 3- to 11-membered, 3- to 10-membered, 3- to 9-membered, 3- to 8-membered, 3- to 7-membered, 3- to 6-membered, 3- to 5-membered, or 3- to 4-membered heterocyclyl optionally substituted with one or more R^{e}, and each R^{e} is independently selected from the group consisting of halogen, cyano, hydroxyl, sulfhydryl, —NH₂, —NO₂, —SO₂-alkyl, —SO₂— haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, L² is a heterocyclyl containing one or more (e.g. 1, 2, 3, 4, 5 or more) heteroatoms (e.g. O, N, S), which is optionally substituted with one or more R^{e}, and each R^{e} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, L² is a heterocyclyl containing one or more (e.g. 1, 2, 3, 4, 5 or more) N atoms, which is optionally substituted with one or more (e.g. 1, 2, 3, 4, 5 or more) R^{e}, and each R^{e} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, L² is a group consisting of

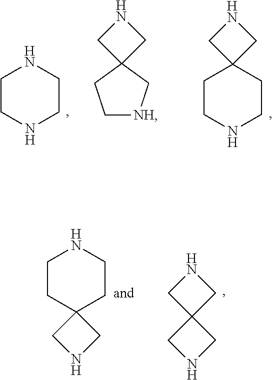

which is optionally substituted with one or more (e.g. 1, 2, 3, 4, 5 or more) R^{e}, and each R^{e} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, L² is a group consisting of

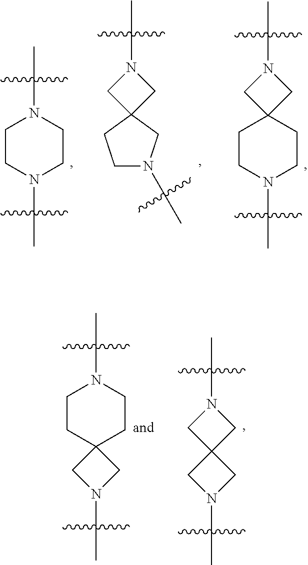

which is optionally substituted with one or more (e.g. 1, 2, 3, 4, 5 or more) R^{e}, and each R^{e} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, L² is a cycloalkyl, which is optionally substituted with one or more (e.g. 1, 2, 3, 4, 5 or more) R^{e}, and each R^{e} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, L² is a 3- to 12-membered, 3- to 11-membered, 3- to 10-membered, 3- to 9-membered, 3- to 8-membered, 3- to 7-membered, 3- to 6-membered, 3- to 5-membered, or 3- to 4-membered cycloalkyl, which is optionally substituted with one or more (e.g. 1, 2, 3, 4, 5 or more) R^{e}, and each R^{e} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, L² is a aryl, which is optionally substituted with one or more (e.g. 1, 2, 3, 4, 5 or more) R^{e}, and each R^{e} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, L² is a 3- to 12-membered, 3- to 11-membered, 3- to 10-membered, 3- to 9-membered, 3- to 8-membered, 3- to 7-membered, 3- to 6-membered, 3- to 5-membered, or 3- to 4-membered aryl, which is optionally substituted with one or more (e.g. 1, 2, 3, 4, 5 or more) R^{e}, and each R^{e} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, L² is a heteroaryl, which is optionally substituted with one or more (e.g. 1, 2, 3, 4, 5 or more) R^{e}, and each R^{e} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, L² is a 3- to 12-membered, 3- to 11-membered, 3- to 10-membered, 3- to 9-membered, 3- to 8-membered, 3- to 7-membered, 3- to 6-membered, 3- to 5-membered, or 3- to 4-membered heteroaryl, which is optionally substituted with one or more (e.g. 1, 2, 3, 4, 5 or more) R^{e}, and each R^{e} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, L² is a heteroaryl containing one or more (e.g. 1, 2, 3, 4, 5 or more) heteroatoms (e.g. O, N, S), which is optionally substituted with one or more (e.g. 1, 2, 3, 4, 5 or more) R^{e}, and each R^{e} is independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl.

In some embodiments, R⁴ is

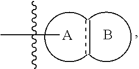

wherein
Ring A is selected from the group consisting of cycloalkyl, heterocyclyl, aryl, and heteroaryl, each of which is optionally substituted with one or more R^{f};
Ring B is selected from the group consisting of cycloalkyl, heterocyclyl, aryl, and heteroaryl, each of which is optionally substituted with one or more R^{g};
[symbol] is a bond via which Ring A is fused to Ring B;
each R^{f} is independently selected from the group consisting of oxo, halogen, cyano, hydroxyl, sulfhydryl, —NH₂, —NO₂, alkyl, alkenyl, alkynyl, heteroalkyl, heteroalkenyl, heteroalkynyl, haloalkyl, alkoxyl, haloalkoxyl, cycloalkyl, heterocyclyl, aryl, heteroaryl, and —S(O)₂—R^{a4};
each R^{g} is independently selected from the group consisting of oxo, halogen, cyano, hydroxyl, sulfhydryl, —NH₂, —NO₂, alkyl, alkenyl, alkynyl, heteroalkyl, heteroalkenyl, heteroalkynyl, haloalkyl, alkoxyl, haloalkoxyl, cycloalkyl, heterocyclyl, aryl, heteroaryl, —NH—C(O)—R^{a5}, —NH—S(O)₂—R^{a5}, —P(O)(R^{a5})₂, —S(O)₂—R^{a5} wherein each of alkyl, alkenyl, alkynyl, heteroalkyl, heteroalkenyl, heteroalkynyl, cycloalkyl, heterocyclyl, aryl, and heteroaryl is optionally substituted with one or more of a group selected from halogen, cyano, hydroxyl, sulfhydryl, —NH₂, —NO₂, alkyl, alkenyl, alkynyl, heteroalkyl, heteroalkenyl, or heteroalkynyl; and
R^{a4} and R^{a5} are each independently selected from the group consisting of alkyl, alkenyl, alkynyl, heteroalkyl, heteroalkenyl, heteroalkynyl, cycloalkyl, heterocyclyl, aryl and heteroaryl, wherein each of cycloalkyl, heterocyclyl, aryl and heteroaryl is optionally substituted with one or more of a group selected from halogen, cyano, hydroxyl, sulfhydryl, —NH₂, —NO₂, alkyl, alkenyl, alkynyl, heteroalkyl, heteroalkenyl, or heteroalkynyl.

In some embodiments, each R^{f} is independently oxo, alkyl, —S(O)₂-alkyl or —S(O)₂-phenyl, wherein the phenyl is optionally substituted with one or more (e.g. 1, 2, 3 or more) alkyl.

In some embodiments, each R^{f} is independently selected from oxo, C₁₋₆ alkyl, —S(O)₂—C₁₋₆ alkyl or —S(O)₂-tolyl.

In some embodiments, each R^{g} is independently selected from the group consisting of hydroxyl, halogen, —NH₂, —NO₂, —NH—C(O)-alkyl, —NH—S(O)₂-alkyl, —P(O)(alkyl)₂, —S(O)₂-aryl, alkyl, alkenyl, cycloalkyl, aryl and heteroaryl, wherein each of alkyl, aryl and heteroaryl is optionally substituted with one or more groups selected from hydroxyl, halogen or alkyl.

In some embodiments, each R^{g}, when present, is independently selected from a group consisting of hydroxyl, halogen, C₁₋₆ alkyl, —NH₂, —NO₂, cyclopentyl, cyclopentenyl, propenyl, phenyl, pyridinyl, pyrazolyl, thienyl, —NH—C(O)—C₁₋₆ alkyl, —NH—S(O)₂—C₁₋₆ alkyl, —P(O)(C₁₋₆ alkyl)₂, C₁₋₆ alkyl substituted with a hydroxyl, and a phenyl substituted with one or more halogen.

In some embodiments, each R^{g}, when present, is independently selected from the group consisting of hydroxyl, halogen, —NH₂, —NO₂, methyl, isopropyl, propenyl, cyclopentyl, cyclopentenyl, phenyl, pyridinyl, pyrazolyl, thienyl, —NH—C(O)-methyl, —NH—S(O)₂-methyl, —P(O)(C₁₋₂ alkyl)₂, —CH(CH₃)CH₂OH, and chlorophenyl.

In some embodiments, each R^{g} is independently a halogen selected from F, Cl, Br, or I.

In some embodiments, Ring A is a cycloalkyl optionally substituted with one or more (e.g. 1, 2, 3, 4, 5 or more) R^{f}.

In some embodiments, Ring A is a C₃₋₁₂ cycloalkyl, C₃₋₁₁ cycloalkyl, C₃₋₁₀ cycloalkyl, C₃₋₉ cycloalkyl, C₃₋₈ cycloalkyl, C₃₋₇ cycloalkyl, C₃₋₆ cycloalkyl, C₃₋₅ cycloalkyl, C₃₋₄ cycloalkyl, C₄₋₁₂ cycloalkyl, C₄₋₁₁ cycloalkyl, C₄₋₁₀ cycloalkyl, C₄₋₉ cycloalkyl, C₄₋₈ cycloalkyl, C₄₋₇ cycloalkyl, C₄₋₆ cycloalkyl, or C₄₋₅ cycloalkyl optionally substituted with one or more (e.g. 1, 2, 3, 4, 5 or more) R^{f}.

In some embodiments, Ring A is

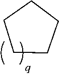

optionally substituted with one or more (e.g. 1, 2, 3, 4, 5 or more) R^{f}, wherein q is 0, 1, 2 or 3. In some embodiments, q is 0. In some embodiments, q is 1. In some embodiments, q is 2. In some embodiments, q is 3.

In some embodiments, Ring A is

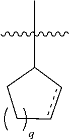

optionally substituted with one or more (e.g. 1, 2, 3, 4, 5 or more) R^{f}, wherein q is 0, 1, 2 or 3, and [symbol] is the bond via which Ring A is fused to Ring B.

In some embodiments, Ring A is a heterocyclyl optionally substituted with one or more R^{f}.

In some embodiments, Ring A is a 3- to 12-membered, 3- to 11-membered, 3- to 10-membered, 3- to 9-membered, 3- to 8-membered, 3- to 7-membered, 3- to 6-membered, 3- to 5-membered, 3- to 4-membered, 4- to 12-membered, 4- to 11-membered, 4- to 10-membered, 4- to 9-membered, 4- to 8-membered, 4- to 7-membered, 4- to 6-membered, or 4- to 5-membered heterocyclyl optionally substituted with one or more (e.g. 1, 2, 3, 4, 5 or more) R^{f}. In some embodiments, Ring A is a 4- to 7-membered heterocyclyl optionally substituted with one or more (e.g. 1, 2, 3, 4, 5 or more) R^{f}.

In some embodiments, Ring A is a heterocyclyl containing one or more (e.g. 1, 2, 3, 4, 5 or more) heteroatoms (e.g. O, N, S), which is optionally substituted with one or more (e.g. 1, 2, 3, 4, 5 or more) R^{f}.

In some embodiments, Ring A is a 4- to 7-membered (e.g. 4-membered, 5-membered, 6-membered, 7-membered) heterocyclyl containing 1, 2 or 3 O atoms, which is optionally substituted with one or more (e.g. 1, 2, 3, 4, 5 or more) R^{f}. In some embodiments, Ring A is a 4- to 7-membered (e.g. 4-membered, 5-membered, 6-membered, 7-membered) heterocyclyl containing 1, 2 or 3 N atoms, which is optionally substituted with one or more (e.g. 1, 2, 3, 4, 5 or more) R^{f}. In some embodiments, Ring A is a 4- to 7-membered (e.g. 4-membered, 5-membered, 6-membered, 7-membered) heterocyclyl containing 1, 2 or 3 S atoms, which is optionally substituted with one or more (e.g. 1, 2, 3, 4, 5 or more) R^{f}.

In some embodiments, Ring A is

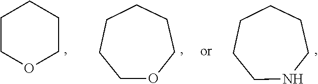

each of which is optionally substituted with one or more (e.g. 1, 2, 3, 4, 5 or more) R^{f}.

In some embodiments, Ring A is selected from the group consisting of:

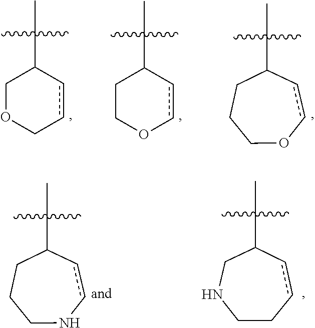

each of which is optionally substituted with one or more (e.g. 1, 2, 3, 4, 5 or more) R^{f}, and wherein [symbol] is the bond via which Ring A is fused to Ring B. in some embodiments, each R^{f} is independently oxo, C₁₋₆ alkyl, —S(O)₂—C₁₋₆ alkyl or —S(O)₂-tolyl.

In some embodiments, Ring A is selected from the group consisting of:

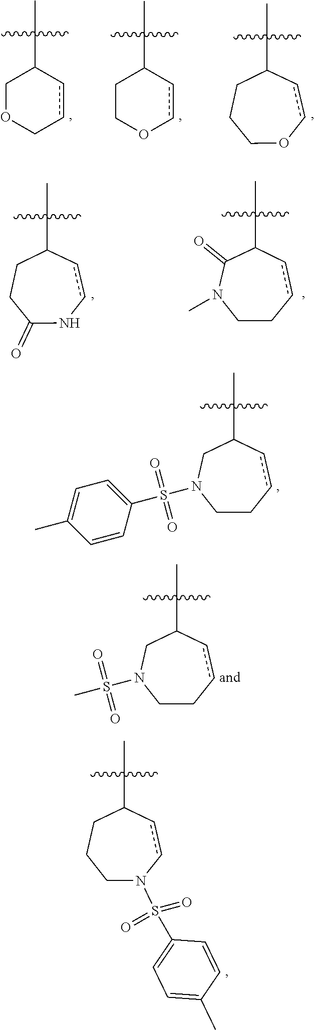

wherein [symbol] is the bond via which Ring A is fused to Ring B.

In some embodiments, Ring B is a cycloalkyl optionally substituted with one or more (e.g. 1, 2, 3, 4, 5 or more) R^{g}.

In some embodiments, Ring B is a C₃₋₁₂ cycloalkyl, C₃₋₁₁ cycloalkyl, C₃₋₁₀ cycloalkyl, C₃₋₉ cycloalkyl, C₃₋₈ cycloalkyl, C₃₋₇ cycloalkyl, C₃₋₆ cycloalkyl, C₃₋₅ cycloalkyl, C₃₋₄ cycloalkyl, C₄₋₁₂ cycloalkyl, C₄₋₁₁ cycloalkyl, C₄₋₁₀ cycloalkyl, C₄₋₉ cycloalkyl, C₄₋₈ cycloalkyl, C₄₋₇ cycloalkyl, C₄₋₆ cycloalkyl, or C₄₋₅ cycloalkyl optionally substituted with one or more (e.g. 1, 2, 3, 4, 5 or more) R^{g}.

In some embodiments, Ring B is a heterocyclyl optionally substituted with one or more R^{g}.

In some embodiments, Ring B is a 3- to 12-membered, 3- to 11-membered, 3- to 10-membered, 3- to 9-membered, 3- to 8-membered, 3- to 7-membered, 3- to 6-membered, 3- to 5-membered, 3- to 4-membered, 4- to 12-membered, 4- to 11-membered, 4- to 10-membered, 4- to 9-membered, 4- to 8-membered, 4- to 7-membered, 4- to 6-membered, or 4- to 5-membered heterocyclyl optionally substituted with one or more (e.g. 1, 2, 3, 4, 5 or more) R^{g}. In some embodiments, Ring B is a 4- to 7-membered heterocyclyl optionally substituted with one or more (e.g. 1, 2, 3, 4, 5 or more) R^{g}.

In some embodiments, Ring B is a heterocyclyl containing one or more (e.g. 1, 2, 3, 4, 5 or more) heteroatoms (e.g. O, N, S), which is optionally substituted with one or more (e.g. 1, 2, 3, 4, 5 or more) R^{g}.

In some embodiments, Ring B is a heteroaryl, optionally substituted with one or more (e.g. 1, 2, 3, 4, 5 or more) R^{g}.

In some embodiments, Ring B is a 3- to 12-membered, 3- to 11-membered, 3- to 10-membered, 3- to 9-membered, 3- to 8-membered, 3- to 7-membered, 3- to 6-membered, 3- to 5-membered, 3- to 4-membered, 4- to 12-membered, 4- to 11-membered, 4- to 10-membered, 4- to 9-membered, 4- to 8-membered, 4- to 7-membered, 4- to 6-membered, or 4- to 5-membered heteroaryl optionally substituted with one or more (e.g. 1, 2, 3, 4, 5 or more) R^{g}. In some embodiments, Ring B is a 4- to 7-membered heteroaryl optionally substituted with one or more (e.g. 1, 2, 3, 4, 5 or more) R^{g}.

In some embodiments, Ring B is a heteroaryl containing one or more (e.g. 1, 2, 3, 4, 5 or more) heteroatoms (e.g. O, N, S), which is optionally substituted with one or more (e.g. 1, 2, 3, 4, 5 or more) R^{g}.

In some embodiments, Ring B is an aryl optionally substituted with one or more (e.g. 1, 2, 3, 4, 5 or more) R^{g}.

In some embodiments, Ring B is a C₃₋₁₂ aryl, C₃₋₁₁ aryl, C₃₋₁₀ aryl, C₃₋₉ aryl, C₃₋₈ aryl, C₃₋₇ aryl, C₃₋₆ aryl, C₃₋₅ aryl, C₃₋₄ aryl, C₄₋₁₂ aryl, C₄₋₁₁ aryl, C₄₋₁₀ aryl, C₄₋₉ aryl, C₄₋₈ aryl, C₄₋₇ aryl, C₄₋₆ aryl, or C₄₋₅ aryl optionally substituted with one or more (e.g. 1, 2, 3, 4, 5 or more) R^{g}.

In some embodiments, Ring B is a phenyl, biphenyl, naphthyl, or anthracyl, each of which is optionally substituted with one or more (e.g. 1, 2, 3, 4, 5 or more) R^{g}.

In some embodiments, Ring B is a phenyl, which is optionally substituted with one or more (e.g. 1, 2, 3, 4, 5 or more) R^{g}, wherein each R^{g} is independently selected from the group consisting of hydroxyl, halogen, —NH₂, —NO₂, —NH—C(O)— alkyl, —NH—S(O)₂-alkyl, —P(O)(alkyl)₂, —S(O)₂-phenyl, alkyl, alkenyl, cycloalkyl, phenyl and heteroaryl, wherein each of alkyl, phenyl and heteroaryl is optionally substituted with one or more group selected from hydroxyl, halogen or alkyl.

In some embodiments, Ring B is an unsubstituted phenyl.

In some embodiments, Ring B is a phenyl substituted with a group selected from the group consisting of hydroxyl, halogen, C₁₋₆ alkyl, —NH₂, —NO₂, cyclopentyl, cyclopentenyl, propenyl, phenyl, pyridinyl, pyrazolyl, thienyl, —NH—C(O)—C₁₋₆ alkyl, —NH—S(O)₂—C₁₋₆ alkyl, —P(O)(C₁₋₆ alkyl)₂, C₁₋₆ alkyl substituted with a hydroxyl, and a phenyl substituted with one or more halogen.

In some embodiments, Ring B is a group selected from the group consisting of:

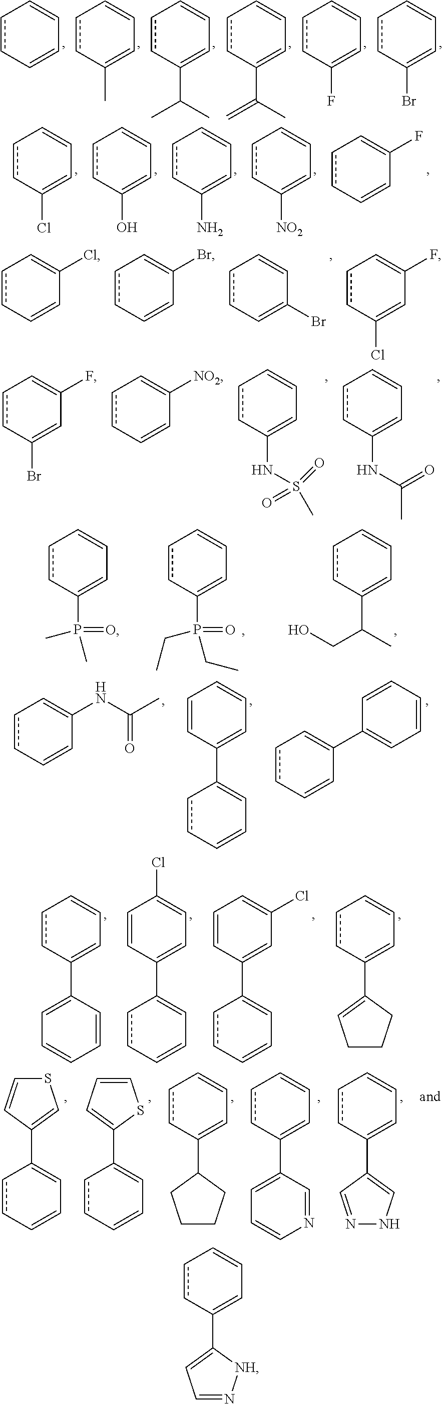

wherein [symbol] is the bond via which Ring B is fused to Ring A.

In some embodiments, [symbol] is a single bond via which Ring B is fused to Ring A. In some embodiments, [symbol] is a double bond via which Ring B is fused to Ring A.

In another aspect, the present disclosure provides a compound having Formula III or Formula IV, a tautomer, a stereoisomer, or a pharmaceutically acceptable salt thereof,

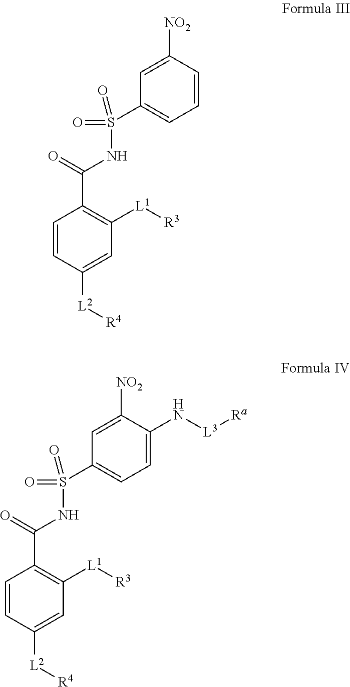

wherein -L¹-R³ is

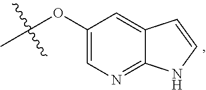 [SMILES: *Oc1cnc2[nH]ccc2c1]

L², L³, R^{a} and R⁴ are as defined supra.

In some embodiments, the present disclosure provides a compound having Formula III or Formula IV as described above, a tautomer, a stereoisomer, or a pharmaceutically acceptable salt thereof, wherein -L¹-R³ is absent, L², L³, R^{a} and R⁴ are as defined supra.

In another aspect, the present disclosure provides a compound having Formula IV(a), Formula IV(b), Formula IV(c), Formula IV(d), or Formula IV(e), or a tautomer, a stereoisomer, or a pharmaceutically acceptable salt thereof,

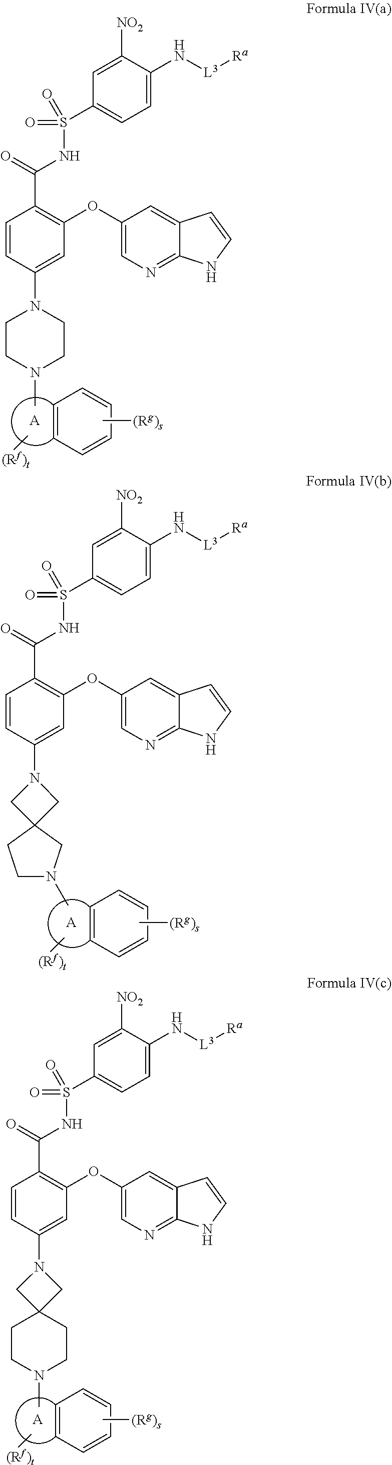

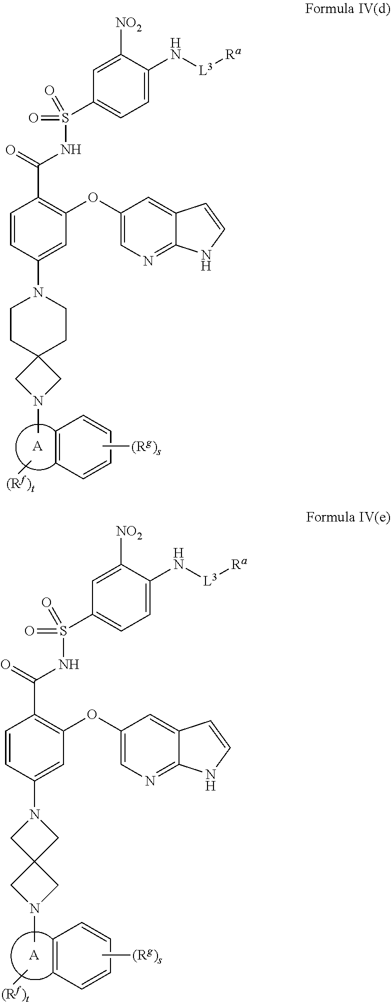

wherein each of s and t is independently 0, 1, 2 or 3, L³, R^{a}, Ring A, R^{f}, R^{g} are as defined supra.

In some embodiments, the present disclosure provides a compound having Formula IV(a), Formula IV(b), Formula IV(c), Formula IV(d), or Formula IV(e) as described above, or a tautomer, a stereoisomer, or a pharmaceutically acceptable salt thereof, wherein L³ is alkyl optionally substituted with one or more R^{b}, and each R^{b} is independently selected from the group consisting of halogen, cyano, hydroxyl, —NH₂, —SO₂-alkyl, —SO₂-haloalkyl, alkyl, haloalkyl, alkoxyl, and haloalkoxyl.

In some embodiments, L³ is C₁₋₆ alkyl optionally substituted with one or more R^{b}, and each R^{b} is independently selected from the group consisting of halogen, cyano, hydroxyl, —NH₂, —SO₂-alkyl, —SO₂-haloalkyl, alkyl, haloalkyl, alkoxyl, and haloalkoxyl.

In some embodiments, R^{a} is independently a cycloalkyl or heterocyclyl.

In some embodiments, Ring A is independently a cycloalkyl or heterocyclyl.

In some embodiments, each R^{f} is independently oxo, alkyl, —S(O)₂-alkyl or —S(O)₂-phenyl, wherein the phenyl is optionally substituted with one or more alkyl.

In some embodiments, each R^{g} is independently selected from the group consisting of hydroxyl, halogen, —NH₂, —NO₂, —NH—C(O)-alkyl, —NH—S(O)₂-alkyl, —P(O)(alkyl)₂, —S(O)₂-aryl, alkyl, alkenyl, cycloalkyl, aryl and heteroaryl, wherein each of alkyl, aryl and heteroaryl is optionally substituted with one or more group selected from hydroxyl, halogen or alkyl.

In some embodiments, s is 0. In some embodiments, s is 1. In some embodiments, s is 2. In some embodiments, s is 3.

In some embodiments, t is 0. In some embodiments, t is 1. In some embodiments, t is 2. In some embodiments, t is 3.

In some embodiments, the present disclosure provides a compound having Formula IV(a), Formula IV(b), Formula IV(c), Formula IV(d), or Formula IV(e) as described above, or a tautomer, a stereoisomer, or a pharmaceutically acceptable salt thereof, wherein R^{a} is selected from the group consisting of:

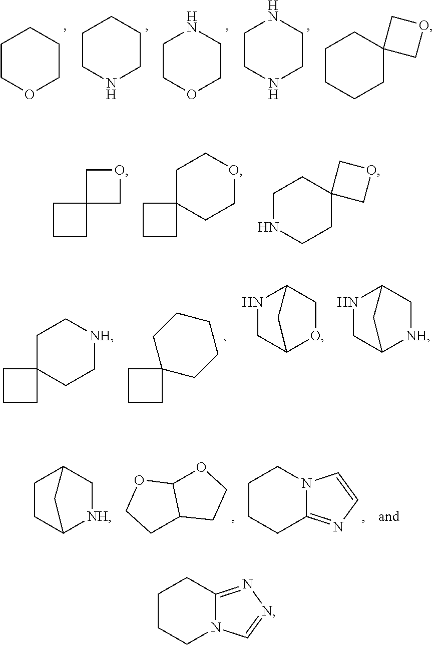

each of which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, haloalkoxyl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3}; R^{a1}, R^{a2} and R^{a3} are each independently selected from the group consisting of hydrogen, hydroxyl, halogen, alkyl, haloalkyl, alkoxyl, cycloalkyl and -alkyl-NH₂.

In some embodiments, the present disclosure provides a compound having Formula IV(a), Formula IV(b), Formula IV(c), Formula IV(d), or Formula IV(e) as described above, or a tautomer, a stereoisomer, or a pharmaceutically acceptable salt thereof, wherein R^{a} is selected from the group consisting of:

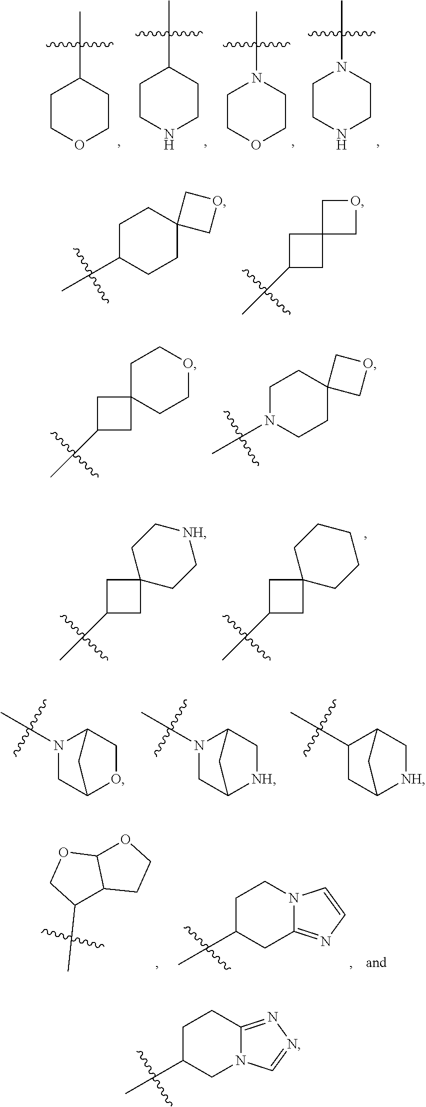

each of which is optionally substituted with one or more R^{c}, wherein each R^{c} is independently selected from the group consisting of halogen, cyano, hydroxyl, sulfhydryl, —NH₂, —NO₂, alkyl, heteroalkyl, haloalkyl, alkoxyl, haloalkoxyl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3}; R^{a1}, R^{a2} and R^{a3} are each independently selected from the group consisting of hydrogen, hydroxyl, halogen, alkyl, haloalkyl, alkoxyl, cycloalkyl and -alkyl-NH₂.

In some embodiments, the present disclosure provides a compound having Formula IV(a), Formula IV(b), Formula IV(c), Formula IV(d), or Formula IV(e) as described above, or a tautomer, a stereoisomer, or a pharmaceutically acceptable salt thereof, wherein R^{a} is selected from the group consisting of:

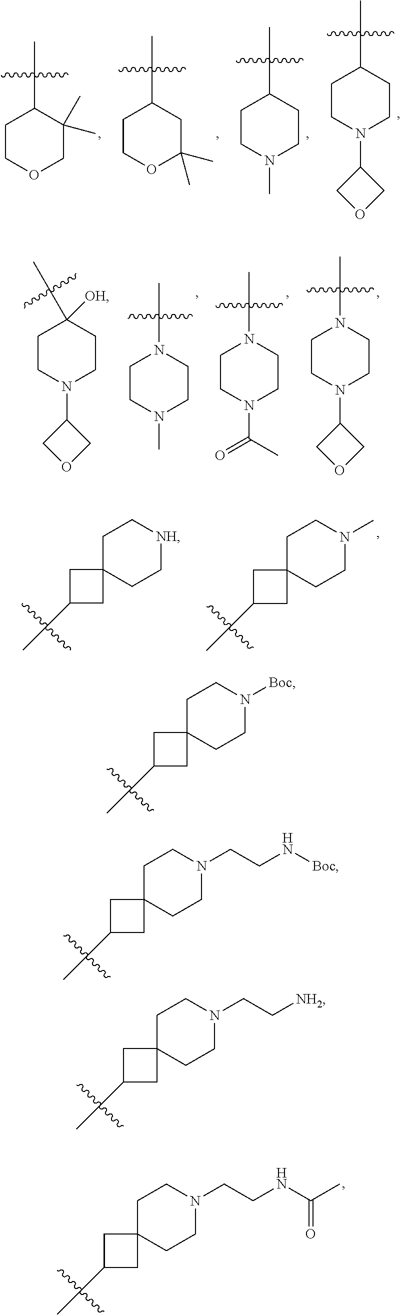

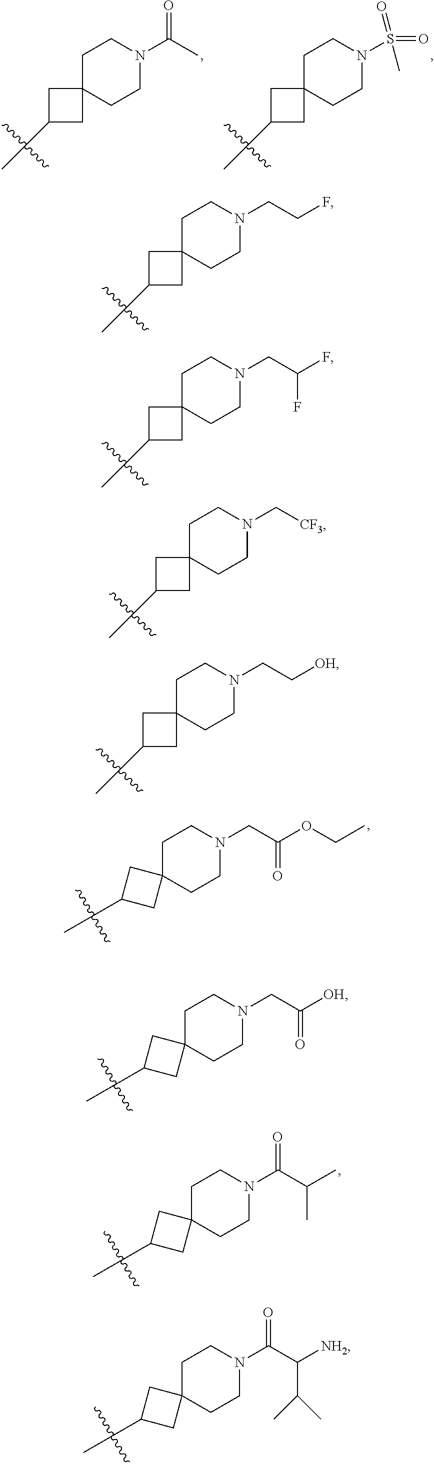

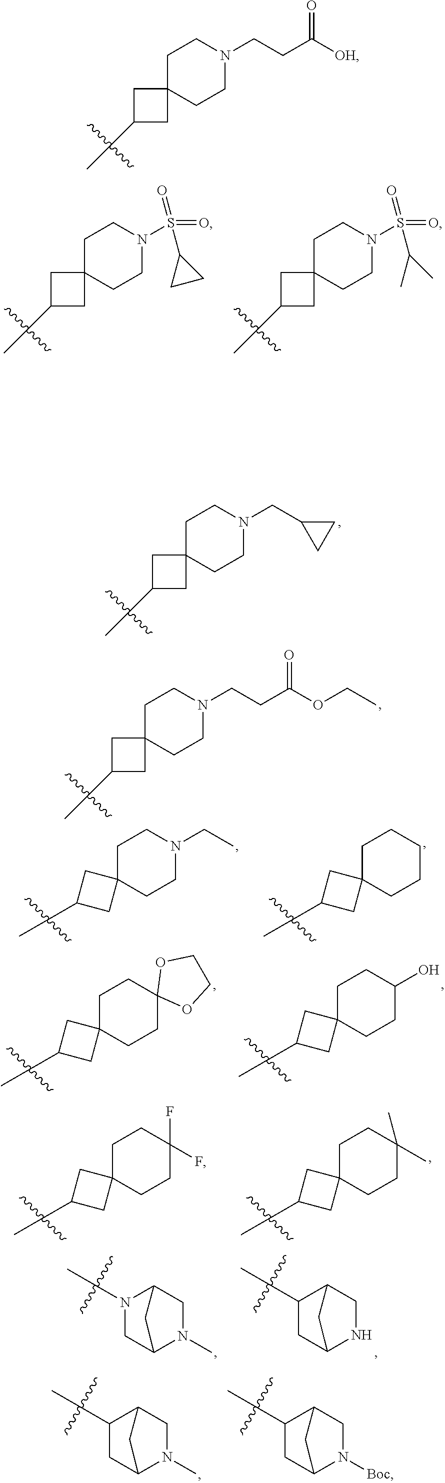

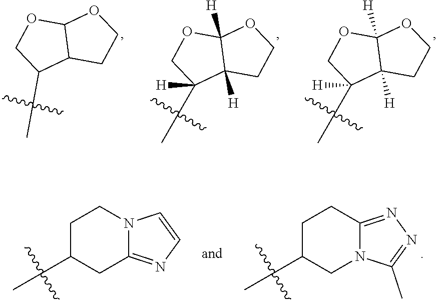 [SMILES: *C1COC2OCCC12 ; *[C@@H]1CO[C@@H]2OCC[C@@H]21 ; *[C@H]1CO[C@H]2OCC[C@H]21 ; *C1CCn2ccnc2C1 ; *C1CCc2nnc(C)n2C1]

In some embodiments, the present disclosure provides a compound having Formula IV(a), Formula IV(b), Formula IV(c), Formula IV(d), or Formula IV(e) as described above, or a tautomer, a stereoisomer, or a pharmaceutically acceptable salt thereof, wherein Ring A is selected from

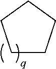

(wherein q is 0, 1, 2 or 3),

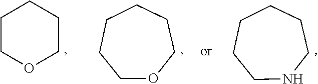

each of which is optionally substituted with one or more (e.g. 1, 2, 3, 4, 5 or more) R^{f}, wherein each R^{f} is independently oxo, C₁₋₆ alkyl, —S(O)₂—C₁₋₆ alkyl or —S(O)₂-tolyl.

In some embodiments, the present disclosure provides a compound having Formula IV(a), Formula IV(b), Formula IV(c), Formula IV(d), or Formula IV(e) as described above, or a tautomer, a stereoisomer, or a pharmaceutically acceptable salt thereof, wherein Ring A is selected from the group consisting of:

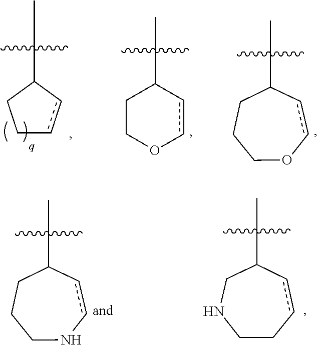

wherein q is 0, 1, 2 or 3, and [symbol] is the bond via which Ring A is fused to Ring B.

In some embodiments, the present disclosure provides a compound having Formula IV(a), Formula IV(b), Formula IV(c), Formula IV(d), or Formula IV(e) as described above, or a tautomer, a stereoisomer, or a pharmaceutically acceptable salt thereof, wherein Ring A is selected from the group consisting of:

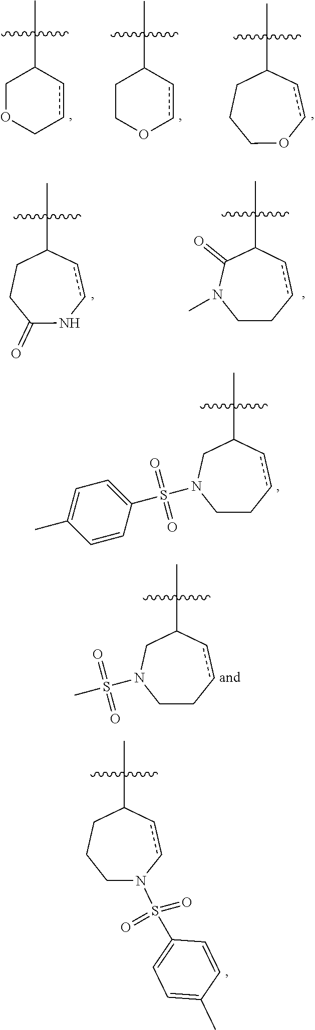

wherein [symbol] is the bond via which Ring A is fused to Ring B.

In another aspect, the present disclosure provides a compound, or a tautomer, a stereoisomer, or a pharmaceutically acceptable salt thereof, selected from the group consisting of:

| Compound | |
|---|---|
| No. | IUPAC Name |
| 1 | 2-((1H-pyrrolo[2,3-b]pyridin-5-yl)oxy)-N-((3-nitrophenyl)sulfonyl)-4- |
| | (4-(1,2,3,4-tetrahydronaphthalen-1-yl)piperazin-1-yl)benzamide |
| 1A | (R)-2-((1H-pyrrolo[2,3-b]pyridin-5-yl)oxy)-N-((3- |
| | nitrophenyl)sulfonyl)-4-(4-(1,2,3,4-tetrahydronaphthalen-1- |
| | yl)piperazin-1-yl)benzamide |
| 1B | (S)-2-((1H-pyrrolo[2,3-b]pyridin-5-yl)oxy)-N-((3- |
| | nitrophenyl)sulfonyl)-4-(4-(1,2,3,4-tetrahydronaphthalen-1- |
| | yl)piperazin-1-yl)benzamide |
| 2 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(1,2,3,4- |
| | tetrahydronaphthalen-1-yl)piperazin-1-yl)benzamide |
| 3 | 2-((1H-pyrrolo[2,3-b]pyridin-5-yl)oxy)-4-(4-(2,3-dihydro-1H-inden-1- |
| | yl)piperazin-1-yl)-N-((3-nitrophenyl)sulfonyl)benzamide |
| 3A | (S)-2-((1H-pyrrolo[2,3-b]pyridin-5-yl)oxy)-4-(4-(2,3-dihydro-1H- |
| | inden-1-yl)piperazin-1-yl)-N-((3-nitrophenyl)sulfonyl)benzamide |
| 3B | (R)-2-((1H-pyrrolo[2,3-b]pyridin-5-yl)oxy)-4-(4-(2,3-dihydro-1H- |
| | inden-1-yl)piperazin-1-yl)-N-((3-nitrophenyl)sulfonyl)benzamide |
| 4 | 2-((1H-pyrrolo[2,3-b]pyridin-5-yl)oxy)-4-(4-(2,3-dihydro-1H-inden-1- |
| | yl)piperazin-1-yl)-N-((3-nitro-4-(((tetrahydro-2H-pyran-4- |
| | yl)methyl)amino)phenyl)sulfonyl)benzamide |
| 5 | 2-((1H-pyrrolo[2,3-b]pyridin-5-yl)oxy)-N-((3-nitrophenyl)sulfonyl)-4- |
| | (4-(6,7,8,9-tetrahydro-5H-benzo[7]annulen-5-yl)piperazin-1- |
| | yl)benzamide |
| 5A | (S)-2-((1H-pyrrolo[2,3-b]pyridin-5-yl)oxy)-N-((3- |
| | nitrophenyl)sulfonyl)-4-(4-(6,7,8,9-tetrahydro-5H-benzo[7]annulen-5- |
| | yl)piperazin-1-yl)benzamide |
| 5B | (R)-2-((1H-pyrrolo[2,3-b]pyridin-5-yl)oxy)-N-((3- |
| | nitrophenyl)sulfonyl)-4-(4-(6,7,8,9-tetrahydro-5H-benzo[7]annulen-5- |
| | yl)piperazin-1-yl)benzamide |
| 6 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(6,7,8,9- |
| | tetrahydro-5H-benzo[7]annulen-5-yl)piperazin-1-yl)benzamide |
| 6A | (S)-2-((1H-pyrrolo[2,3-b]pyridin-5-yl)oxy)-N-((3-nitro-4- |
| | (((tetrahydro-2H-pyran-4-yl)methyl)amino)phenyl)sulfonyl)-4-(4- |
| | (6,7,8,9-tetrahydro-5H-benzo[7]annulen-5-yl)piperazin-1- |
| | yl)benzamide |
| 6B | (R)-2-((1H-pyrrolo[2,3-b]pyridin-5-yl)oxy)-N-((3-nitro-4- |
| | (((tetrahydro-2H-pyran-4-yl)methyl)amino)phenyl)sulfonyl)-4-(4- |
| | (6,7,8,9-tetrahydro-5H-benzo[7]annulen-5-yl)piperazin-1- |
| | yl)benzamide |
| 7 | 2-((1H-pyrrolo[2,3-b]pyridin-5-yl)oxy)-N-((3-nitrophenyl)sulfonyl)-4- |
| | (4-(7-phenyl-2,3-dihydro-1H-inden-1-yl)piperazin-1-yl)benzamide |
| 8 | 2-((1H-pyrrolo[2,3-b]pyridin-5-yl)oxy)-N-((3-nitrophenyl)sulfonyl)-4- |
| | (4-(8-phenyl-1,2,3,4-tetrahydronaphthalen-1-yl)piperazin-1- |
| | yl)benzamide |
| 9 | 2-((1H-pyrrolo[2,3-b]pyridin-5-yl)oxy)-4-(4-(chroman-4-yl)piperazin- |
| | 1-yl)-N-((3-nitrophenyl)sulfonyl)benzamide |
| 10 | 2-((1H-pyrrolo[2,3-b]pyridin-5-yl)oxy)-4-(4-(isochroman-4- |
| | yl)piperazin-1-yl)-N-((3-nitrophenyl)sulfonyl)benzamide |
| 11 | 2-((1H-pyrrolo[2,3-b]pyridin-5-yl)oxy)-N-((3-nitrophenyl)sulfonyl)-4- |
| | (4-(8-(thiophen-3-yl)-1,2,3,4-tetrahydronaphthalen-1-yl)piperazin-1- |
| | yl)benzamide |
| 12 | 2-((1H-pyrrolo[2,3-b]pyridin-5-yl)oxy)-4-(4-(isochroman-4- |
| | yl)piperazin-1-yl)-N-((3-nitro-4-(((tetrahydro-2H-pyran-4- |
| | yl)methyl)amino)phenyl)sulfonyl)benzamide |
| 13 | 2-((1H-pyrrolo[2,3-b]pyridin-5-yl)oxy)-4-(4-(8-(4-chlorophenyl)- |
| | 1,2,3,4-tetrahydronaphthalen-1-yl)piperazin-1-yl)-N-((3- |
| | nitrophenyl)sulfonyl)benzamide |
| 14 | 2-((1H-pyrrolo[2,3-b]pyridin-5-yl)oxy)-4-(4-(8-(3-chlorophenyl)- |
| | 1,2,3,4-tetrahydronaphthalen-1-yl)piperazin-1-yl)-N-((3- |
| | nitrophenyl)sulfonyl)benzamide |
| 15 | 2-((1H-pyrrolo[2,3-b]pyridin-5-yl)oxy)-N-((3-nitrophenyl)sulfonyl)-4- |
| | (4-(8-(thiophen-2-yl)-1,2,3,4-tetrahydronaphthalen-1-yl)piperazin-1- |
| | yl)benzamide |
| 16 | 2-((1H-pyrrolo[2,3-b]pyridin-5-yl)oxy)-4-(4-(8-(4-chlorophenyl)- |
| | 1,2,3,4-tetrahydronaphthalen-1-yl)piperazin-1-yl)-N-((3-nitro-4- |
| | (((tetrahydro-2H-pyran-4-yl)methyl)amino)phenyl)sulfonyl)benzamide |
| 17 | 2-((1H-pyrrolo[2,3-b]pyridin-5-yl)oxy)-4-(4-(chroman-4-yl)piperazin- |
| | 1-yl)-N-((3-nitro-4-(((tetrahydro-2H-pyran-4- |
| | yl)methyl)amino)phenyl)sulfonyl)benzamide |
| 18 | 2-((1H-pyrrolo[2,3-b]pyridin-5-yl)oxy)-4-(4-(1-bromo-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 18A | (R)-2-((1H-pyrrolo[2,3-b]pyridin-5-yl)oxy)-4-(4-(1-bromo-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 18B | (S)-2-((1H-pyrrolo[2,3-b]pyridin-5-yl)oxy)-4-(4-(1-bromo-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 19 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(1-phenyl-6,7,8,9- |
| | tetrahydro-5H-benzo[7]annulen-5-yl)piperazin-1-yl)benzamide |
| 20 | 2-((1H-pyrrolo[2,3-b]pyridin-5-yl)oxy)-4-(4-(1-methyl-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 21 | 2-((1H-pyrrolo[2,3-b]pyridin-5-yl)oxy)-4-(4-(3-bromo-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 22 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(3-phenyl-6,7,8,9- |
| | tetrahydro-5H-benzo[7]annulen-5-yl)piperazin-1-yl)benzamide |
| 23 | 2-((1H-pyrrolo[2,3-b]pyridin-5-yl)oxy)-4-(4-(2-bromo-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 24 | (R)-N-((4-(((7-(2-acetamidoethyl)-7-azaspiro[3.5]nonan-2- |
| | yl)methyl)amino)-3-nitrophenyl)sulfonyl)-4-(4-(1-chloro-6,7,8,9- |
| | tetrahydro-5H-benzo[7]annulen-5-yl)piperazin-1-yl)benzamide |
| 25 | 2-((1H-pyrrolo[2,3-b]pyridin-5-yl)oxy)-4-(4-(2-chloro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 26 | 2-((1H-pyrrolo[2,3-b]pyridin-5-yl)oxy)-4-(4-(3-chloro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 27 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 27A | (S)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 27B | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 28 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(2-oxo-2,3,4,5- |
| | tetrahydro-1H-benzo[b]azepin-5-yl)piperazin-1-yl)benzamide |
| 29 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((1- |
| | methylpiperidin-4-yl)methyl)amino)-3- |
| | nitrophenyl)sulfonyl)benzamide |
| 30 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(3-nitro-6,7,8,9- |
| | tetrahydro-5H-benzo[7]annulen-5-yl)piperazin-1-yl)benzamide |
| 31 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-((2- |
| | morpholinoethyl)amino)-3-nitrophenyl)sulfonyl)benzamide |
| 31A | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-((2- |
| | morpholinoethyl)amino)-3-nitrophenyl)sulfonyl)benzamide |
| 31B | (S)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-((2- |
| | morpholinoethyl)amino)-3-nitrophenyl)sulfonyl)benzamide |
| 32 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-((2-(4- |
| | methylpiperazin-1-yl)ethyl)amino)-3-nitrophenyl)sulfonyl)benzamide |
| 33 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(1-nitro-6,7,8,9- |
| | tetrahydro-5H-benzo[7]annulen-5-yl)piperazin-1-yl)benzamide |
| 34 | 2-((1H-pyrrolo[2,3-b]pyridin-5-yl)oxy)-4-(4-(3-acetamido-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 35 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(3-tosyl-2,3,4,5- |
| | tetrahydro-1H-benzo[d]azepin-1-yl)piperazin-1-yl)benzamide |
| 36 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(2,3,4,5- |
| | tetrahydrobenzo[b]oxepin-5-yl)piperazin-1-yl)benzamide |
| 37 | 2-((1H-pyrrolo[2,3-b]pyridin-5-yl)oxy)-4-(4-(3-fluoro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 38 | 2-((1H-pyrrolo[2,3-b]pyridin-5-yl)oxy)-N-((4-(((2-oxaspiro[3.5]nonan- |
| | 7-yl)methyl)amino)-3-nitrophenyl)sulfonyl)-4-(4-(1-chloro-6,7,8,9- |
| | tetrahydro-5H-benzo[7]annulen-5-yl)piperazin-1-yl)benzamide |
| 39 | 2-((1H-pyrrolo[2,3-b]pyridin-5-yl)oxy)-N-((4-((2-(4-acetylpiperazin-1- |
| | yl)ethyl)amino)-3-nitrophenyl)sulfonyl)-4-(4-(1-chloro-6,7,8,9- |
| | tetrahydro-5H-benzo[7]annulen-5-yl)piperazin-1-yl)benzamide |
| 40 | (R)-2-((1H-pyrrolo[2,3-b]pyridin-5-yl)oxy)-4-(4-(1-hydroxy-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 41 | 2-((1H-pyrrolo[2,3-b]pyridin-5-yl)oxy)-N-((4-((2-(2-oxa-5- |
| | azabicyclo[2.2.1 ]heptan-5-yl)ethyl)amino)-3-nitrophenyl)sulfonyl)-4- |
| | (4-(1-chloro-6,7,8,9-tetrahydro-5H-benzo[7]annulen-5-yl)piperazin-1- |
| | yl)benzamide |
| 42 | 2-((1H-pyrrolo[2,3-b]pyridin-5-yl)oxy)-N-((4-((2-(2-oxa-7- |
| | azaspiro[3.5]nonan-7-yl)ethyl)amino)-3-nitrophenyl)sulfonyl)-4-(4-(1- |
| | chloro-6,7,8,9-tetrahydro-5H-benzo[7]annulen-5-yl)piperazin-1- |
| | yl)benzamide |
| 43 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-((2-(5- |
| | methyl-2,5-diazabicyclo[2.2.1]heptan-2-yl)ethyl)amino)-3- |
| | nitrophenyl)sulfonyl)benzamide |
| 44 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((2- |
| | methyl-2-azabicyclo[2.2.1]heptan-5-yl)methyl)amino)-3- |
| | nitrophenyl)sulfonyl)benzamide |
| 45 | 2-((1H-pyrrolo[2,3-b]pyridin-5-yl)oxy)-4-(4-(3-methyl-2-oxo-2,3,4,5- |
| | tetrahydro-1H-benzo[d]azepin-1-yl)piperazin-1-yl)-N-((3-nitro-4- |
| | (((tetrahydro-2H-pyran-4-yl)methyl)amino)phenyl)sulfonyl)benzamide |
| 46 | 2-((1H-pyrrolo[2,3-b]pyridin-5-yl)oxy)-N-((4-(((2- |
| | oxaspiro[3.3]heptan-6-yl)methyl)amino)-3-nitrophenyl)sulfonyl)-4-(4- |
| | (1-chloro-6,7,8,9-tetrahydro-5H-benzo[7]annulen-5-yl)piperazin-1- |
| | yl)benzamide |
| 47 | 2-((1H-pyrrolo[2,3-b]pyridin-5-yl)oxy)-4-(4-(1-(cyclopent-1-en-1-yl)- |
| | 6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 48 | (R)-4-(4-(1-chloro-6,7,8,9-tetrahydro-5H-benzo[7]annulen-5- |
| | yl)piperazin-1-yl)-N-((4-(((7-(methylsulfonyl)-7-azaspiro[3.5]nonan-2- |
| | yl)methyl)amino)-3-nitrophenyl)sulfonyl)benzamide |
| 49 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(1-(pyridin-3-yl)- |
| | 6,7,8,9-tetrahydro-5H-benzo[7]annulen-5-yl)piperazin-1-yl)benzamide |
| 50 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(1-(prop-1-en-2- |
| | yl)-6,7,8,9-tetrahydro-5H-benzo[7]annulen-5-yl)piperazin-1- |
| | yl)benzamide |
| 51 | 2-((1H-pyrrolo[2,3-b]pyridin-5-yl)oxy)-4-(4-(1-isopropyl-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 52 | 4-(4-(1-(1H-pyrazol-4-yl)-6,7,8,9-tetrahydro-5H-benzo[7]annulen-5- |
| | yl)piperazin-1-yl)-2-((1H-pyrrolo[2,3-b]pyridin-5-yl)oxy)-N-((3-nitro- |
| | 4-(((tetrahydro-2H-pyran-4- |
| | yl)methyl)amino)phenyl)sulfonyl)benzamide |
| 53 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((5,6,7,8-tetrahydroimidazo[1,2-a]pyridin-7- |
| | yl)methyl)amino)phenyl)sulfonyl)benzamide |
| 54 | 4-(4-(1-(1H-pyrazol-3-yl)-6,7,8,9-tetrahydro-5H-benzo[7]annulen-5- |
| | yl)piperazin-1-yl)-2-((1H-pyrrolo[2,3-b]pyridin-5-yl)oxy)-N-((3-nitro- |
| | 4-(((tetrahydro-2H-pyran-4- |
| | yl)methyl)amino)phenyl)sulfonyl)benzamide |
| 55 | 2-((1H-pyrrolo[2,3-b]pyridin-5-yl)oxy)-4-(4-(1-cyclopentyl-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 56 | 2-((1H-pyrrolo[2,3-b]pyridin-5-yl)oxy)-4-(4-(1-(methylsulfonamido)- |
| | 6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 57 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((3- |
| | methyl-5,6,7,8-tetrahydro-[1,2,4]triazolo[4,3-a]pyridin-6- |
| | yl)methyl)amino)-3-nitrophenyl)sulfonyl)benzamide |
| 58 | 2-((1H-pyrrolo[2,3-b]pyridin-5-yl)oxy)-4-(4-(1-amino-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 59 | 2-((1H-pyrrolo[2,3-b]pyridin-5-yl)oxy)-4-(4-(1-acetamido-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 60 | 2-((1H-pyrrolo[2,3-b]pyridin-5-yl)oxy)-4-(4-(3-(methylsulfonyl)- |
| | 2,3,4,5-tetrahydro-1H-benzo[d]azepin-1-yl)piperazin-1-yl)-N-((3-nitro- |
| | 4-(((tetrahydro-2H-pyran-4- |
| | yl)methyl)amino)phenyl)sulfonyl)benzamide |
| 61 | 2-((1H-pyrrolo[2,3-b]pyridin-5-yl)oxy)-4-(4-(1-(1-hydroxypropan-2- |
| | yl)-6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 62 | 2-((1H-pyrrolo[2,3-b]pyridin-5-yl)oxy)-4-(4-(1-(diethylphosphoryl)- |
| | 6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 63 | 2-((1H-pyrrolo[2,3-b]pyridin-5-yl)oxy)-4-(4-(1-(dimethylphosphoryl)- |
| | 6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 64 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4- |
| | (((hexahydrofuro[2,3-b]furan-3-yl)methyl)amino)-3- |
| | nitrophenyl)sulfonyl)benzamide |
| 65 | (R)-2-((1H-pyrrolo[2,3-b]pyridin-5-yl)oxy)-N-((4-((2-(4- |
| | acetylpiperazin-1-yl)ethyl)amino)-3-nitrophenyl)sulfonyl)-4-(4-(1- |
| | bromo-6,7,8,9-tetrahydro-5H-benzo[7]annulen-5-yl)piperazin-1- |
| | yl)benzamide |
| 66 | (R)-2-((1H-pyrrolo[2,3-b]pyridin-5-yl)oxy)-4-(4-(1-bromo-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 67 | (R)-2-((1H-pyrrolo[2,3-b]pyridin-5-yl)oxy)-N-((4-(((2- |
| | oxaspiro[3.5]nonan-7-yl)methyl)amino)-3-nitrophenyl)sulfonyl)-4-(4- |
| | (1-bromo-6,7,8,9-tetrahydro-5H-benzo[7]annulen-5-yl)piperazin-1- |
| | yl)benzamide |
| 68 | (R)-2-((1H-pyrrolo[2,3-b]pyridin-5-yl)oxy)-N-((4-((2-(2-oxa-7- |
| | azaspiro[3.5]nonan-7-yl)ethyl)amino)-3-nitrophenyl)sulfonyl)-4-(4-(1- |
| | bromo-6,7,8,9-tetrahydro-5H-benzo[7]annulen-5-yl)piperazin-1- |
| | yl)benzamide |
| 69 | (R)-2-((1H-pyrrolo[2,3-b]pyridin-5-yl)oxy)-4-(4-(1-bromo-6,7,8,9- |
| | tetrahydro-5H-benzo[7]annulen-5-yl)piperazin-1-yl)-N-((4-(((1- |
| | methylpiperidin-4-yl)methyl)amino)-3- |
| | nitrophenyl)sulfonyl)benzamide |
| 70 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 71 | (R)-2-((1H-pyrrolo[2,3-b]pyridin-5-yl)oxy)-N-((4-(((2- |
| | oxaspiro[3.5]nonan-7-yl)methyl)amino)-3-nitrophenyl)sulfonyl)-4-(4- |
| | (1-chloro-6,7,8,9-tetrahydro-5H-benzo[7]annulen-5-yl)piperazin-1- |
| | yl)benzamide |
| 72 | (R)-2-((1H-pyrrolo[2,3-b]pyridin-5-yl)oxy)-N-((4-(((2- |
| | oxaspiro[3.3]heptan-6-yl)methyl)amino)-3-nitrophenyl)sulfonyl)-4-(4- |
| | (1-chloro-6,7,8,9-tetrahydro-5H-benzo[7]annulen-5-yl)piperazin-1- |
| | yl)benzamide |
| 73 | (R)-2-((1H-pyrrolo[2,3-b]pyridin-5-yl)oxy)-N-((3-nitro-4- |
| | (((tetrahydro-2H-pyran-4-yl)methyl)amino)phenyl)sulfonyl)-4-(4-(1- |
| | nitro-6,7,8,9-tetrahydro-5H-benzo[7]annulen-5-yl)piperazin-1- |
| | yl)benzamide |
| 74 | (R)-2-((1H-pyrrolo[2,3-b]pyridin-5-yl)oxy)-N-((4-(((2- |
| | oxaspiro[3.5]nonan-7-yl)methyl)amino)-3-nitrophenyl)sulfonyl)-4-(4- |
| | (1-nitro-6,7,8,9-tetrahydro-5H-benzo[7]annulen-5-yl)piperazin-1- |
| | yl)benzamide |
| 75 | (R)-2-((1H-pyrrolo[2,3-b]pyridin-5-yl)oxy)-N-((4-(((2- |
| | oxaspiro[3.3]heptan-6-yl)methyl)amino)-3-nitrophenyl)sulfonyl)-4-(4- |
| | (1-nitro-6,7,8,9-tetrahydro-5H-benzo[7]annulen-5-yl)piperazin-1- |
| | yl)benzamide |
| 76 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(1-tosyl-2,3,4,5- |
| | tetrahydro-1H-benzo[b]azepin-5-yl)piperazin-1-yl)benzamide |
| 77 | (R)-2-((1H-pyrrolo[2,3-b]pyridin-5-yl)oxy)-N-((4-(((2- |
| | oxaspiro[3.3]heptan-6-yl)methyl)amino)-3-nitrophenyl)sulfonyl)-4-(4- |
| | (1-bromo-6,7,8,9-tetrahydro-5H-benzo[7]annulen-5-yl)piperazin-1- |
| | yl)benzamide |
| 78 | (R)-2-((1H-pyrrolo[2,3-b]pyridin-5-yl)oxy)-4-(4-(1-bromo-3-fluoro- |
| | 6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 79 | (R)-2-((1H-pyrrolo[2,3-b]pyridin-5-yl)oxy)-N-((4-(((2- |
| | oxaspiro[3.5]nonan-7-yl)methyl)amino)-3-nitrophenyl)sulfonyl)-4-(4- |
| | (1-bromo-3-fluoro-6,7,8,9-tetrahydro-5H-benzo[7]annulen-5- |
| | yl)piperazin-1-yl)benzamide |
| 80 | (R)-2-((1H-pyrrolo[2,3-b]pyridin-5-yl)oxy)-N-((4-((2-(2-oxa-7- |
| | azaspiro[3.5]nonan-7-yl)ethyl)amino)-3-nitrophenyl)sulfonyl)-4-(4-(1- |
| | bromo-3-fluoro-6,7,8,9-tetrahydro-5H-benzo[7]annulen-5- |
| | yl)piperazin-1-yl)benzamide |
| 81 | 2-((1H-pyrrolo[2,3-b]pyridin-5-yl)oxy)-4-(4-((R)-1-bromo-3-fluoro- |
| | 6,7,8,9-tetrahydro-5H-benzo[7]annulen-5-yl)piperazin-1-yl)-N-((4- |
| | ((((3R,3aR,6aS)-hexahydrofuro[2,3-b]furan-3-yl)methyl)amino)-3- |
| | nitrophenyl)sulfonyl)benzamide |
| 82 | 2-((1H-pyrrolo[2,3-b]pyridin-5-yl)oxy)-4-(4-((R)-1-bromo-3-fluoro- |
| | 6,7,8,9-tetrahydro-5H-benzo[7]annulen-5-yl)piperazin-1-yl)-N-((4- |
| | ((((3S,3aS,6aR)-hexahydrofuro[2,3-b]furan-3-yl)methyl)amino)-3- |
| | nitrophenyl)sulfonyl)benzamide |
| 83 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((1-(oxetan-3-yl)piperidin-4- |
| | yl)methyl)amino)phenyl)sulfonyl)benzamide |
| 84 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4-((2- |
| | (4-(oxetan-3-yl)piperazin-1-yl)ethyl)amino)phenyl)sulfonyl)benzamide |
| 85 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((4- |
| | hydroxy-1-(oxetan-3-yl)piperidin-4-yl)methyl)amino)-3- |
| | nitrophenyl)sulfonyl)benzamide |
| 86 | (R)-2-((1H-pyrrolo[2,3-b]pyridin-5-yl)oxy)-4-(4-(1-chloro-3-fluoro- |
| | 6,7,8,9-tetrahydro-5H-benzo[7]annulen-5-yl)piperazin-1-yl)-N-((4- |
| | (((1-methylpiperidin-4-yl)methyl)amino)-3- |
| | nitrophenyl)sulfonyl)benzamide |
| 87 | (R)-2-((1H-pyrrolo[2,3-b]pyridin-5-yl)oxy)-4-(4-(1-chloro-3-fluoro- |
| | 6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 88 | (R)-2-((1H-pyrrolo[2,3-b]pyridin-5-yl)oxy)-4-(4-(1-chloro-3-fluoro- |
| | 6,7,8,9-tetrahydro-5H-benzo[7]annulen-5-yl)piperazin-1-yl)-N-((4- |
| | (((4-hydroxy-1-(oxetan-3-yl)piperidin-4-yl)methyl)amino)-3- |
| | nitrophenyl)sulfonyl)benzamide |
| 89 | (R)-2-((1H-pyrrolo[2,3-b]pyridin-5-yl)oxy)-4-(4-(1-fluoro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 90 | (R)-2-((1H-pyrrolo[2,3-b]pyridin-5-yl)oxy)-4-(4-(1-methyl-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 91 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(6-(1,2,3,4- |
| | tetrahydronaphthalen-1-yl)-2,6-diazaspiro[3.4]octan-2-yl)benzamide |
| 92 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(7-(1,2,3,4- |
| | tetrahydronaphthalen-1-yl)-2,7-diazaspiro[3.5]nonan-2-yl)benzamide |
| 93 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(2-(6,7,8,9- |
| | tetrahydro-5H-benzo[7]annulen-5-yl)-2,7-diazaspiro[3.5]nonan-7- |
| | yl)benzamide |
| 94 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(2-(1,2,3,4- |
| | tetrahydronaphthalen-1-yl)-2,7-diazaspiro[3.5]nonan-7-yl)benzamide |
| 95 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(6-(6,7,8,9- |
| | tetrahydro-5H-benzo[7]annulen-5-yl)-2,6-diazaspiro[3.3]heptan-2- |
| | yl)benzamide |
| 96 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(6-(1,2,3,4- |
| | tetrahydronaphthalen-1-yl)-2,6-diazaspiro[3.3]heptan-2-yl)benzamide |
| 97 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(7-(6,7,8,9- |
| | tetrahydro-5H-benzo[7]annulen-5-yl)-2,7-diazaspiro[3.5]nonan-2- |
| | yl)benzamide |
| 98 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(6-(6,7,8,9- |
| | tetrahydro-5H-benzo[7]annulen-5-yl)-2,6-diazaspiro[3.4]octan-2- |
| | yl)benzamide |
| 99 | (R)-2-((1H-pyrrolo[2,3-b]pyridin-5-yl)oxy)-N-((4-(((7- |
| | oxaspiro[3.5]nonan-2-yl)methyl)amino)-3-nitrophenyl)sulfonyl)-4-(4- |
| | (1-chloro-6,7,8,9-tetrahydro-5H-benzo[7]annulen-5-yl)piperazin-1- |
| | yl)benzamide |
| 100 | tert-butyl (R)-2-(((4-(N-(2-((1H-pyrrolo[2,3-b]pyridin-5-yl)oxy)-4-(4- |
| | (1-chloro-6,7,8,9-tetrahydro-5H-benzo[7]annulen-5-yl)piperazin-1- |
| | yl)benzoyl)sulfamoyl)-2-nitrophenyl)amino)methyl)-7- |
| | azaspiro[3.5]nonane-7-carboxylate |
| 101 | 2-((1H-pyrrolo[2,3-b]pyridin-5-yl)oxy)-4-(4-((R)-1-chloro-6,7,8,9- |
| | tetrahydro-5H-benzo[7]annulen-5-yl)piperazin-1-yl)-N-((4-(((3,3- |
| | dimethyltetrahydro-2H-pyran-4-yl)methyl)amino)-3- |
| | nitrophenyl)sulfonyl)benzamide |
| 102 | 2-((1H-pyrrolo[2,3-b]pyridin-5-yl)oxy)-4-(4-((R)-1-chloro-6,7,8,9- |
| | tetrahydro-5H-benzo[7]annulen-5-yl)piperazin-1-yl)-N-((4-(((2,2- |
| | dimethyltetrahydro-2H-pyran-4-yl)methyl)amino)-3- |
| | nitrophenyl)sulfonyl)benzamide |
| 103 | tert-butyl 5-(((4-(N-(2-((1H-pyrrolo[2,3-b]pyridin-5-yl)oxy)-4-(4-((R)- |
| | 1-chloro-6,7,8,9-tetrahydro-5H-benzo[7]annulen-5-yl)piperazin-1- |
| | yl)benzoyl)sulfamoyl)-2-nitrophenyl)amino)methyl)-2- |
| | azabicyclo[2.2.1]heptane-2-carboxylate |
| 104 | (R)-2-((1H-pyrrolo[2,3-b]pyridin-5-yl)oxy)-N-((4-(((7- |
| | azaspiro[3.5]nonan-2-yl)methyl)amino)-3-nitrophenyl)sulfonyl)-4-(4- |
| | (1-chloro-6,7,8,9-tetrahydro-5H-benzo[7]annulen-5-yl)piperazin-1- |
| | yl)benzamide |
| 105 | 2-((1H-pyrrolo[2,3-b]pyridin-5-yl)oxy)-N-((4-(((2- |
| | azabicyclo[2.2.1]heptan-5-yl)methyl)amino)-3-nitrophenyl)sulfonyl)- |
| | 4-(4-((R)-1-chloro-6,7,8,9-tetrahydro-5H-benzo[7]annulen-5- |
| | yl)piperazin-1-yl)benzamide |
| 106 | 2-((1H-pyrrolo[2,3-b]pyridin-5-yl)oxy)-4-(4-((R)-1-chloro-6,7,8,9- |
| | tetrahydro-5H-benzo[7]annulen-5-yl)piperazin-1-yl)-N-((4-(((2- |
| | methyl-2-azabicyclo[2.2.1]heptan-5-yl)methyl)amino)-3- |
| | nitrophenyl)sulfonyl)benzamide |
| 107 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | (methylsulfonyl)-7-azaspiro[3.5]nonan-2-yl)methyl)amino)-3- |
| | nitrophenyl)sulfonyl)benzamide |
| 108 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | methyl-7-azaspiro[3.5]nonan-2-yl)methyl)amino)-3- |
| | nitrophenyl)sulfonyl)benzamide |
| 109 | tert-butyl (R)-(2-(2-(((4-(N-(2-((1H-pyrrolo[2,3-b]pyridin-5-yl)oxy)-4- |
| | (4-(1-chloro-6,7,8,9-tetrahydro-5H-benzo[7]annulen-5-yl)piperazin-1- |
| | yl)benzoyl)sulfamoyl)-2-nitrophenyl)amino)methyl)-7- |
| | azaspiro[3.5]nonan-7-yl)ethyl)carbamate |
| 110 | (R)-2-((1H-pyrrolo[2,3-b]pyridin-5-yl)oxy)-N-((4-(((7-(2-aminoethyl)- |
| | 7-azaspiro[3.5]nonan-2-yl)methyl)amino)-3-nitrophenyl)sulfonyl)-4- |
| | (4-(1-chloro-6,7,8,9-tetrahydro-5H-benzo[7]annulen-5-yl)piperazin-1- |
| | yl)benzamide |
| 111 | (R)-2-((1H-pyrrolo[2,3-b]pyridin-5-yl)oxy)-N-((4-(((7-acetyl-7- |
| | azaspiro[3.5]nonan-2-yl)methyl)amino)-3-nitrophenyl)sulfonyl)-4-(4- |
| | (1-chloro-6,7,8,9-tetrahydro-5H-benzo[7]annulen-5-yl)piperazin-1- |
| | yl)benzamide |
| 112 | (R)-2-((1H-pyrrolo[2,3-b]pyridin-5-yl)oxy)-N-((4-(((7-(2- |
| | acetamidoethyl)-7-azaspiro[3.5]nonan-2-yl)methyl)amino)-3- |
| | nitrophenyl)sulfonyl)-4-(4-(1-chloro-6,7,8,9-tetrahydro-5H- |
| | benzo[7]annulen-5-yl)piperazin-1-yl)benzamide |
| 113 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7-(2,2- |
| | difluoroethyl)-7-azaspiro[3.5]nonan-2-yl)methyl)amino)-3- |
| | nitrophenyl)sulfonyl)benzamide |
| 114 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7-(2- |
| | fluoroethyl)-7-azaspiro[3.5]nonan-2-yl)methyl)amino)-3- |
| | nitrophenyl)sulfonyl)benzamide |
| 115 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((7-(2,2,2-trifluoroethyl)-7-azaspiro[3.5]nonan-2- |
| | yl)methyl)amino)phenyl)sulfonyl)benzamide |
| 116 | tert-butyl (R)-2-(((4-(N-(4-(4-(1-chloro-6,7,8,9-tetrahydro-5H- |
| | benzo[7]annulen-5-yl)piperazin-1-yl)benzoyl)sulfamoyl)-2- |
| | nitrophenyl)amino)methyl)-7-azaspiro[3.5]nonane-7-carboxylate |
| 117 | (R)-N-((4-(((7-azaspiro[3.5]nonan-2-yl)methyl)amino)-3- |
| | nitrophenyl)sulfonyl)-4-(4-(1-chloro-6,7,8,9-tetrahydro-5H- |
| | benzo[7]annulen-5-yl)piperazin-1-yl)benzamide |
| 118 | (R)-N-((4-(((7-acetyl-7-azaspiro[3.5]nonan-2-yl)methyl)amino)-3- |
| | nitrophenyl)sulfonyl)-4-(4-(1-chloro-6,7,8,9-tetrahydro-5H- |
| | benzo[7]annulen-5-yl)piperazin-1-yl)benzamide |
| 119 | tert-butyl (R)-(2-(2-(((4-(N-(4-(4-(1-chloro-6,7,8,9-tetrahydro-5H- |
| | benzo[7]annulen-5-yl)piperazin-1-yl)benzoyl)sulfamoyl)-2- |
| | nitrophenyl)amino)methyl)-7-azaspiro[3.5]nonan-7-yl)ethyl)carbamate |
| 120 | (R)-N-((4-(((7-(2-aminoethyl)-7-azaspiro[3.5]nonan-2- |
| | yl)methyl)amino)-3-nitrophenyl)sulfonyl)-4-(4-(1-chloro-6,7,8,9- |
| | tetrahydro-5H-benzo[7]annulen-5-yl)piperazin-1-yl)benzamide |
| 121 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7-(2- |
| | hydroxyethyl)-7-azaspiro[3.5]nonan-2-yl)methyl)amino)-3- |
| | nitrophenyl)sulfonyl)benzamide |
| 122 | ethyl (R)-2-(2-(((4-(N-(2-((1H-pyrrolo[2,3-b]pyridin-5-yl)oxy)-4-(4- |
| | (1-chloro-6,7,8,9-tetrahydro-5H-benzo[7]annulen-5-yl)piperazin-1- |
| | yl)benzoyl)sulfamoyl)-2-nitrophenyl)amino)methyl)-7- |
| | azaspiro[3.5]nonan-7-yl)acetate |
| 123 | (R)-2-(2-(((4-(N-(2-((1H-pyrrolo[2,3-b]pyridin-5-yl)oxy)-4-(4-(1- |
| | chloro-6,7,8,9-tetrahydro-5H-benzo[7]annulen-5-yl)piperazin-1- |
| | yl)benzoyl)sulfamoyl)-2-nitrophenyl)amino)methyl)-7- |
| | azaspiro[3.5]nonan-7-yl)acetic acid |
| 124 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | isobutyryl-7-azaspiro[3.5]nonan-2-yl)methyl)amino)-3- |
| | nitrophenyl)sulfonyl)benzamide |
| 125 | 2-((1H-pyrrolo[2,3-b]pyridin-5-yl)oxy)-N-((4-(((7-(L-valyl)-7- |
| | azaspiro[3.5]nonan-2-yl)methyl)amino)-3-nitrophenyl)sulfonyl)-4-(4- |
| | ((R)-1-chloro-6,7,8,9-tetrahydro-5H-benzo[7]annulen-5-yl)piperazin- |
| | 1-yl)benzamide |
| 126 | (R)-3-(2-(((4-(N-(2-((1H-pyrrolo[2,3-b]pyridin-5-yl)oxy)-4-(4-(1- |
| | chloro-6,7,8,9-tetrahydro-5H-benzo[7]annulen-5-yl)piperazin-1- |
| | yl)benzoyl)sulfamoyl)-2-nitrophenyl)amino)methyl)-7- |
| | azaspiro[3.5]nonan-7-yl)propanoic acid |
| 127 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | (cyclopropylsulfonyl)-7-azaspiro[3.5]nonan-2-yl)methyl)amino)-3- |
| | nitrophenyl)sulfonyl)benzamide |
| 128 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | (isopropylsulfonyl)-7-azaspiro[3.5]nonan-2-yl)methyl)amino)-3- |
| | nitrophenyl)sulfonyl)benzamide |
| 129 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | (cyclopropylmethyl)-7-azaspiro[3.5]nonan-2-yl)methyl)amino)-3- |
| | nitrophenyl)sulfonyl)benzamide |
| 130 | ethyl (R)-3-(2-(((4-(N-(2-((1H-pyrrolo[2,3-b]pyridin-5-yl)oxy)-4-(4- |
| | (1-chloro-6,7,8,9-tetrahydro-5H-benzo[7]annulen-5-yl)piperazin-1- |
| | yl)benzoyl)sulfamoyl)-2-nitrophenyl)amino)methyl)-7- |
| | azaspiro[3.5]nonan-7-yl)propanoate |
| 131 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7-ethyl- |
| | 7-azaspiro[3.5]nonan-2-yl)methyl)amino)-3- |
| | nitrophenyl)sulfonyl)benzamide |
| 132 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | ((spiro[3.5]nonan-2-ylmethyl)amino)phenyl)sulfonyl)benzamide |
| 133 | (R)-2-((1H-pyrrolo[2,3-b]pyridin-5-yl)oxy)-N-((4-(((8,11- |
| | dioxadispiro[3.2.47.24]tridecan-2-yl)methyl)amino)-3- |
| | nitrophenyl)sulfonyl)-4-(4-(1-chloro-6,7,8,9-tetrahydro-5H- |
| | benzo[7]annulen-5-yl)piperazin-1-yl)benzamide |
| 134 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | hydroxyspiro[3.5]nonan-2-yl)methyl)amino)-3- |
| | nitrophenyl)sulfonyl)benzamide |
| 135 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7,7- |
| | difluorospiro[3.5]nonan-2-yl)methyl)amino)-3- |
| | nitrophenyl)sulfonyl)benzamide |
| 136 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7,7- |
| | dimethylspiro[3.5]nonan-2-yl)methyl)amino)-3- |
| | nitrophenyl)sulfonyl)benzamide |

Exemplary compounds of the present disclosure are set forth in Table 1 below.

TABLE 1
Structures and Names of Exemplary Compounds

| Compound | | |
|---|---|---|
| No. | Structure | IUPAC Name |
| 1 | 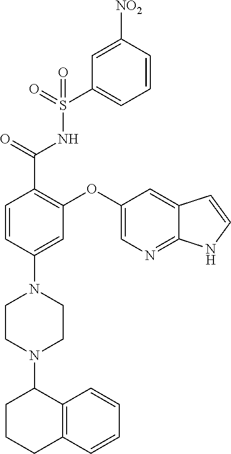 [SMILES: O=C(NS(=O)(=O)c1cccc([N+](=O)[O-])c1)c1ccc(N2CCN(C3CCCc4ccccc43)CC2)cc1Oc1cnc2[nH]ccc2c1] | 2-((1H-pyrrolo[2,3-b]pyridin-5- yl)oxy)-N-((3-nitrophenyl)sulfonyl)-4- (4-(1,2,3,4-tetrahydronaphthalen-1- yl)piperazin-1-yl)benzamide |
| 1A | 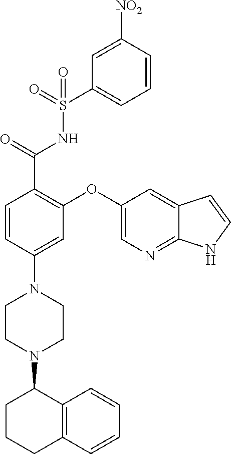 [SMILES: O=C(NS(=O)(=O)c1cccc([N+](=O)[O-])c1)c1ccc(N2CCN([C@@H]3CCCc4ccccc43)CC2)cc1Oc1cnc2[nH]ccc2c1] | (R)-2-((1H-pyrrolo[2,3-b]pyridin-5- yl)oxy)-N-((3-nitrophenyl)sulfonyl)-4- (4-(1,2,3,4-tetrahydronaphthalen-1- y1)piperazin-1-yl)benzamide |
| 1B | 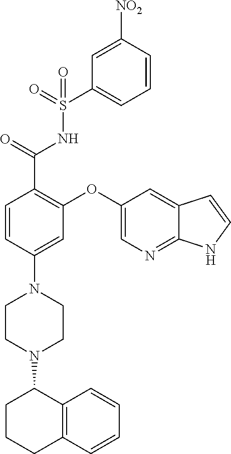 [SMILES: O=C(NS(=O)(=O)c1cccc([N+](=O)[O-])c1)c1ccc(N2CCN([C@H]3CCCc4ccccc43)CC2)cc1Oc1cnc2[nH]ccc2c1] | (S)-2-((1H-pyrrolo[2,3-b]pyridin-5- yl)oxy)-N-((3-nitrophenyl)sulfonyl)-4- (4-(1,2,3,4-tetrahydronaphthalen-1- yl)piperazin-1-yl)benzamide |
| 2 | 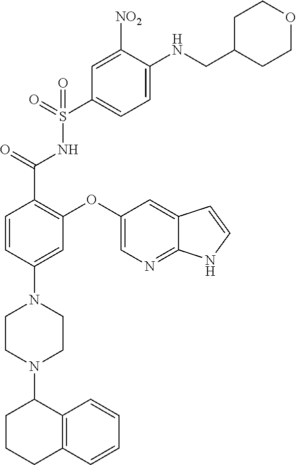 [SMILES: O=C(NS(=O)(=O)c1ccc(NCC2CCOCC2)c([N+](=O)[O-])c1)c1ccc(N2CCN(C3CCCc4ccccc43)CC2)cc1Oc1cnc2[nH]ccc2c1] | 2-((1H-pyrrolo[2,3-b]pyridin-5- yl)oxy)-N-((3-nitro-4-(((tetrahydro- 2H-pyran-4- y1)methyl)amino)phenyl)sulfonyl)-4- (4-(1,2,3,4-tetrahydronaphthalen-1- yl)piperazin-1-yl)benzamide |
| 3 | 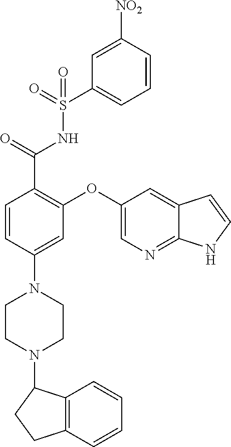 [SMILES: O=C(NS(=O)(=O)c1cccc([N+](=O)[O-])c1)c1ccc(N2CCN(C3CCc4ccccc43)CC2)cc1Oc1cnc2[nH]ccc2c1] | 2-((1H-pyrrolo[2,3-b]pyridin-5- yl)oxy)-4-(4-(2,3-dihydro-1H-inden-1- yl)piperazin-1-yl)-N-((3- nitrophenyl)sulfonyl)benzamide |
| 3A | 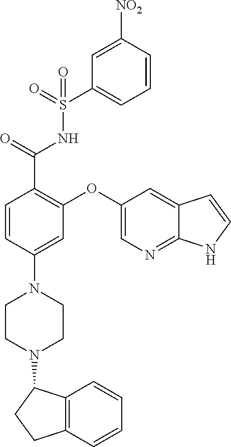 [SMILES: O=C(NS(=O)(=O)c1cccc([N+](=O)[O-])c1)c1ccc(N2CCN([C@H]3CCc4ccccc43)CC2)cc1Oc1cnc2[nH]ccc2c1] | (S)-2-((1H-pyrrolo[2,3-b]pyridin-5- yl)oxy)-4-(4-(2,3-dihydro-1H-inden-1- yl)piperazin-1-yl)-N-((3- nitrophenyl)sulfonyl)benzamide |
| 3B | 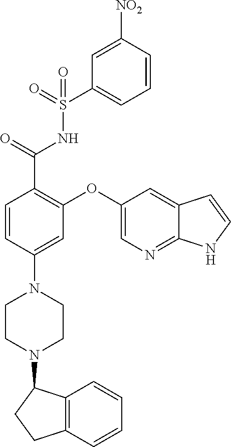 [SMILES: O=C(NS(=O)(=O)c1cccc([N+](=O)[O-])c1)c1ccc(N2CCN([C@@H]3CCc4ccccc43)CC2)cc1Oc1cnc2[nH]ccc2c1] | (R)-2-((1H-pyrrolo[2,3-b]pyridin-5- yl)oxy)-4-(4-(2,3-dihydro-1H-inden-1- yl)piperazin-1-yl)-N-((3- nitrophenyl)sulfonyl)benzamide |
| 4 | 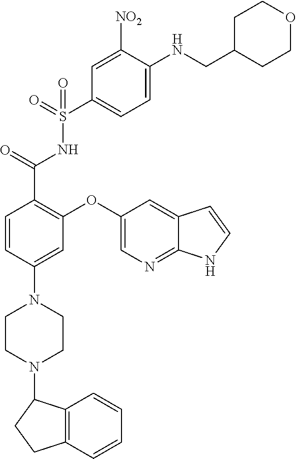 [SMILES: O=C(NS(=O)(=O)c1ccc(NCC2CCOCC2)c([N+](=O)[O-])c1)c1ccc(N2CCN(C3CCc4ccccc43)CC2)cc1Oc1cnc2[nH]ccc2c1] | 2-((1H-pyrrolo[2,3-b]pyridin-5- yl)oxy)-4-(4-(2,3-dihydro-1H-inden-1- yl)piperazin-1-yl)-N-((3-nitro-4- (((tetrahydro-2H-pyran-4- yl)methyl)amino)phenyl)sulfonyl)benz- amide |
| 5 | 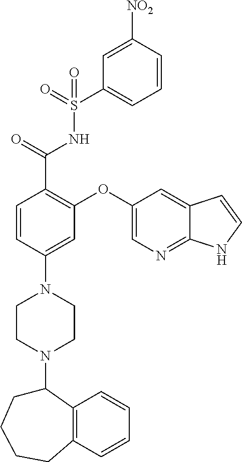 [SMILES: O=C(NS(=O)(=O)c1cccc([N+](=O)[O-])c1)c1ccc(N2CCN(C3CCCCc4ccccc43)CC2)cc1Oc1cnc2[nH]ccc2c1] | 2-((1H-pyrrolo[2,3-b]pyridin-5- yl)oxy)-N-((3-nitrophenyl)sulfony1)-4- (4-(6,7,8,9-tetrahydro-5H- benzo[7]annulen-5-y1)piperazin-1- yl)benzamide |
| 5A | 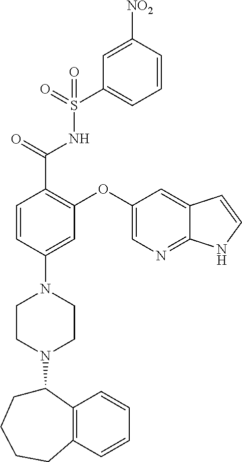 [SMILES: O=C(NS(=O)(=O)c1cccc([N+](=O)[O-])c1)c1ccc(N2CCN([C@H]3CCCCc4ccccc43)CC2)cc1Oc1cnc2[nH]ccc2c1] | (S)-2-((1H-pyrrolo[2,3-b]pyridin-5- yl)oxy)-N-((3-nitrophenyl)sulfonyl)-4- (4-(6,7,8,9-tetrahydro-5H- benzo[7]annulen-5-yl)piperazin-1- yl)benzamide |
| 5B | 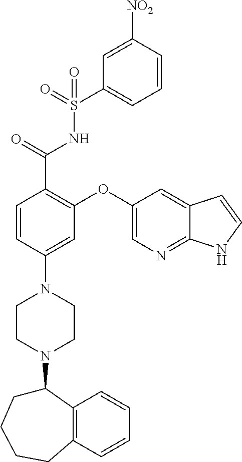 [SMILES: O=C(NS(=O)(=O)c1cccc([N+](=O)[O-])c1)c1ccc(N2CCN([C@@H]3CCCCc4ccccc43)CC2)cc1Oc1cnc2[nH]ccc2c1] | (R)-2-((1H-pyrrolo[2,3-b]pyridin-5- yl)oxy)-N-((3-nitrophenyl)sulfony1)-4- (4-(6,7,8,9-tetrahydro-5H- benzo[7]annulen-5-y1)piperazin-1- yl)benzamide |
| 6 | 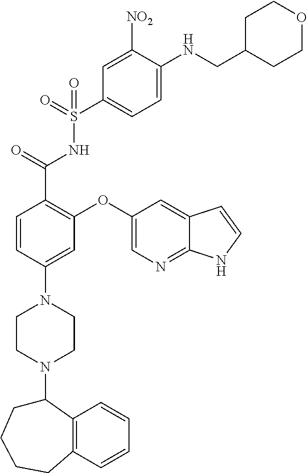 [SMILES: O=C(NS(=O)(=O)c1ccc(NCC2CCOCC2)c([N+](=O)[O-])c1)c1ccc(N2CCN(C3CCCCc4ccccc43)CC2)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(6,7,8,9-tetrahydro-5H- benzo[7]annulen-5-yl)piperazin-1- yl)benzamide |
| 6A | 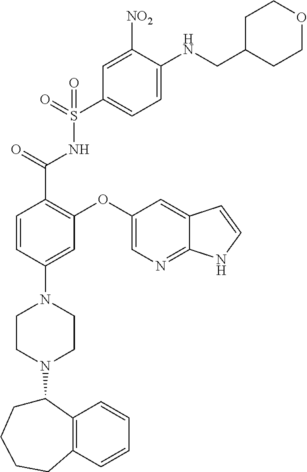 [SMILES: O=C(NS(=O)(=O)c1ccc(NCC2CCOCC2)c([N+](=O)[O-])c1)c1ccc(N2CCN([C@H]3CCCCc4ccccc43)CC2)cc1Oc1cnc2[nH]ccc2c1] | (S)-2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(6,7,8,9-tetrahydro-5H- benzo[7]annulen-5-yl)piperazin-1- yl)benzamide |
| 6B | 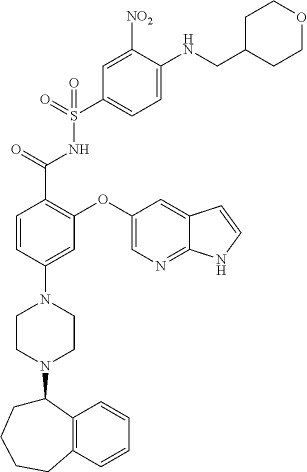 [SMILES: O=C(NS(=O)(=O)c1ccc(NCC2CCOCC2)c([N+](=O)[O-])c1)c1ccc(N2CCN([C@@H]3CCCCc4ccccc43)CC2)cc1Oc1cnc2[nH]ccc2c1] | (R)-2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(6,7,8,9-tetrahydro-5H- benzo[7]annulen-5-yl)piperazin-1- yl)benzamide |
| 7 | 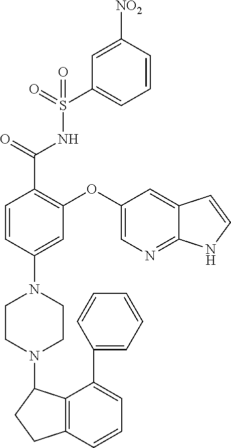 [SMILES: O=C(NS(=O)(=O)c1cccc([N+](=O)[O-])c1)c1ccc(N2CCN(C3CCc4cccc(-c5ccccc5)c43)CC2)cc1Oc1cnc2[nH]ccc2c1] | 2-((1H-pyrrolo[2,3-b]pyridin-5- yl)oxy)-N-((3-nitrophenyl)sulfonyl)-4- (4-(7-phenyl-2,3-dihydro-1H-inden-1- yl)piperazin-1-yl)benzamide |
| 8 | 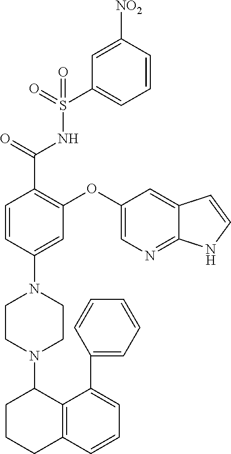 [SMILES: O=C(NS(=O)(=O)c1cccc([N+](=O)[O-])c1)c1ccc(N2CCN(C3CCCc4cccc(-c5ccccc5)c43)CC2)cc1Oc1cnc2[nH]ccc2c1] | 2-((1H-pyrrolo[2,3-b]pyridin-5- yl)oxy)-N-((3-nitrophenyl)sulfonyl)-4- (4-(8-phenyl-1,2,3,4- tetrahydronaphthalen-1-yl)piperazin- 1-yl)benzamide |
| 9 | 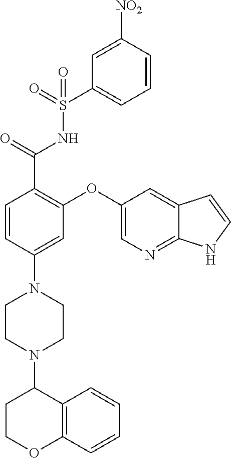 [SMILES: O=C(NS(=O)(=O)c1cccc([N+](=O)[O-])c1)c1ccc(N2CCN(C3CCOc4ccccc43)CC2)cc1Oc1cnc2[nH]ccc2c1] | 2-((1H-pyrrolo[2,3-b]pyridin-5- yl)oxy)-4-(4-(chroman-4-yl)piperazin- 1-y1)-N-((3- nitrophenyl)sulfonyl)benzamide |
| 10 | 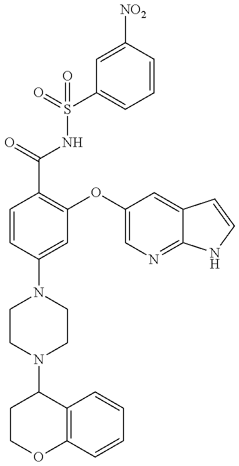 [SMILES: O=C(NS(=O)(=O)c1cccc([N+](=O)[O-])c1)c1ccc(N2CCN(C3CCOc4ccccc43)CC2)cc1Oc1cnc2[nH]ccc2c1] | 2-((1H-pyrrolo[2,3-b]pyridin-5- yl)oxy)-4-(4-(isochroman-4- yl)piperazin-1-y1)-N-((3- nitrophenyl)sulfonyl)benzamide |
| 11 | 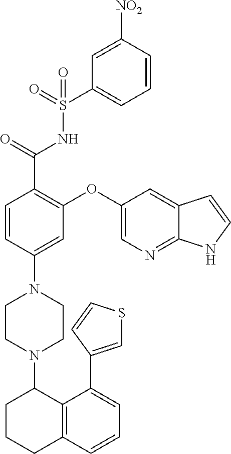 [SMILES: O=C(NS(=O)(=O)c1cccc([N+](=O)[O-])c1)c1ccc(N2CCN(C3CCCc4cccc(-c5ccsc5)c43)CC2)cc1Oc1cnc2[nH]ccc2c1] | 2-((1H-pyrrolo[2,3-b]pyridin-5- yl)oxy)-N-((3-nitrophenyl)sulfonyl)-4- (4-(8-(thiophen-3-y1)-1,2,3,4- tetrahydronaphthalen-1-yl)piperazin- 1-yl)benzamide |
| 12 | 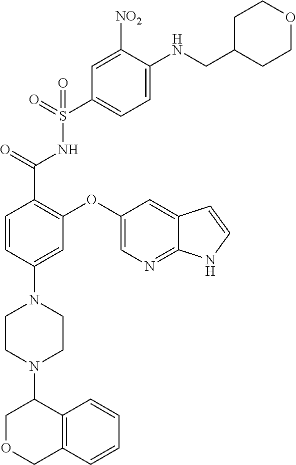 [SMILES: O=C(NS(=O)(=O)c1ccc(NCC2CCOCC2)c([N+](=O)[O-])c1)c1ccc(N2CCN(C3COCc4ccccc43)CC2)cc1Oc1cnc2[nH]ccc2c1] | 2-((1H-pyrrolo[2,3-b]pyridin-5- yl)oxy)-4-(4-(isochroman-4- yl)piperazin-1-yl)-N-((3-nitro-4- (((tetrahydro-2H-pyran-4- yl)methyl)amino)phenyl)sulfonyl)benz- amide |
| 13 | 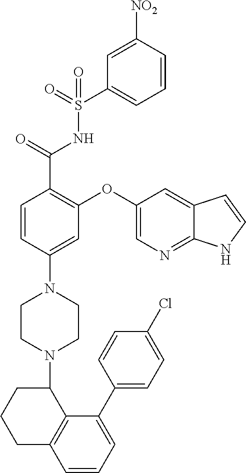 [SMILES: O=C(NS(=O)(=O)c1cccc([N+](=O)[O-])c1)c1ccc(N2CCN(C3CCCc4cccc(-c5ccc(Cl)cc5)c43)CC2)cc1Oc1cnc2[nH]ccc2c1] | 2-((1H-pyrrolo[2,3-b]pyridin-5- yl)oxy)-4-(4-(8-(4-chlorophenyl)- 1,2,3,4-tetrahydronaphthalen-1- yl)piperazin-1-yl)-N-((3- nitrophenyl)sulfonyl)benzamide |
| 14 | 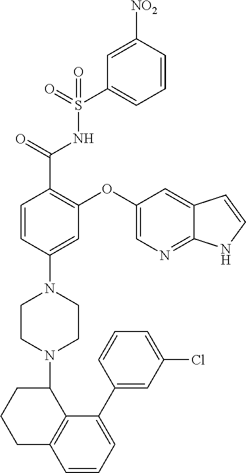 [SMILES: O=C(NS(=O)(=O)c1cccc([N+](=O)[O-])c1)c1ccc(N2CCN(C3CCCc4cccc(-c5cccc(Cl)c5)c43)CC2)cc1Oc1cnc2[nH]ccc2c1] | 2-((1H-pyrrolo[2,3-b]pyridin-5- yl)oxy)-4-(4-(8-(3-chlorophenyl)- 1,2,3,4-tetrahydronaphthalen-1- yl)piperazin-1-y1)-N-((3- nitrophenyl)sulfonyl)benzamide |
| 15 | 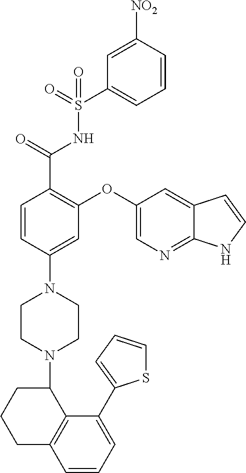 [SMILES: O=C(NS(=O)(=O)c1cccc([N+](=O)[O-])c1)c1ccc(N2CCN(C3CCCc4cccc(-c5cccs5)c43)CC2)cc1Oc1cnc2[nH]ccc2c1] | 2-((1H-pyrrolo[2,3-b]pyridin-5- yl)oxy)-N-((3-nitrophenyl)sulfonyl)-4- (4-(8-(thiophen-2-y1)-1,2,3,4- tetrahydronaphthalen-1-yl)piperazin- 1-yl)benzamide |
| 16 | 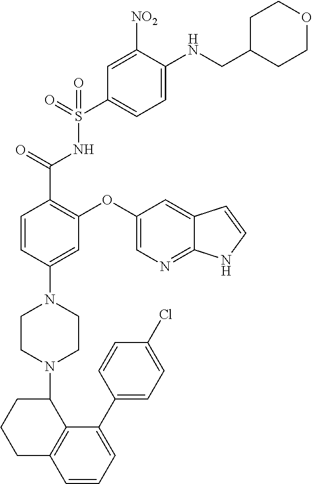 [SMILES: O=C(NS(=O)(=O)c1ccc(NCC2CCOCC2)c([N+](=O)[O-])c1)c1ccc(N2CCN(C3CCCc4cccc(-c5ccc(Cl)cc5)c43)CC2)cc1Oc1cnc2[nH]ccc2c1] | 2-((1H-pyrrolo[2,3-b]pyridin-5- yl)oxy)-4-(4-(8-(4-chlorophenyl)- 1,2,3,4-tetrahydronaphthalen-1- yl)piperazin-1-yl)-N-((3-nitro-4- (((tetrahydro-2H-pyran-4- yl)methyl)amino)phenyl)sulfonyl)benz- amide |
| 17 | 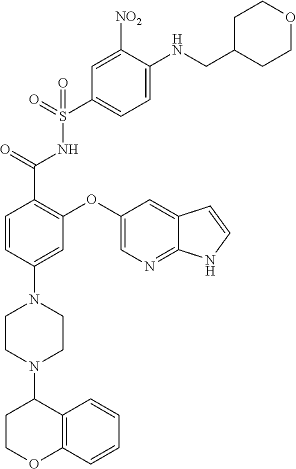 [SMILES: O=C(NS(=O)(=O)c1ccc(NCC2CCOCC2)c([N+](=O)[O-])c1)c1ccc(N2CCN(C3CCOc4ccccc43)CC2)cc1Oc1cnc2[nH]ccc2c1] | 2-((1H-pyrrolo[2,3-b]pyridin-5- yl)oxy)-4-(4-(chroman-4-yl)piperazin- 1-y1)-N-((3-nitro-4-(((tetrahydro-2H- pyran-4- yl)methyl)amino)phenyl)sulfonyl)benz- amide |
| 18 | 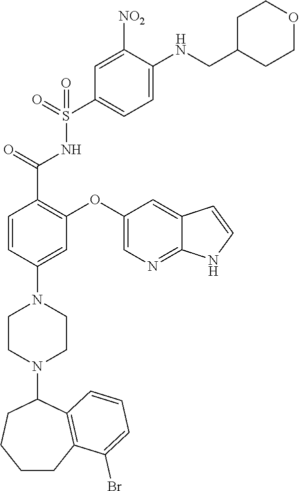 [SMILES: O=C(NS(=O)(=O)c1ccc(NCC2CCOCC2)c([N+](=O)[O-])c1)c1ccc(N2CCN(C3CCCCc4c(Br)cccc43)CC2)cc1Oc1cnc2[nH]ccc2c1] | 2-((1H-pyrrolo[2,3-b]pyridin-5- yl)oxy)-4-(4-(1-bromo-6,7,8,9- tetrahydro-5H-benzo[7]annulen-5- yl)piperazin-1-y1)-N-((3-nitro-4- (((tetrahydro-2H-pyran-4- yl)methyl)amino)phenyl)sulfonyl)benz- amide |
| 18A | 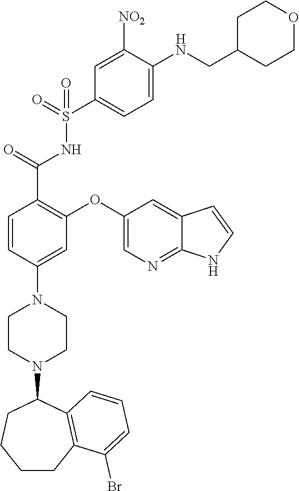 [SMILES: O=C(NS(=O)(=O)c1ccc(NCC2CCOCC2)c([N+](=O)[O-])c1)c1ccc(N2CCN([C@@H]3CCCCc4c(Br)cccc43)CC2)cc1Oc1cnc2[nH]ccc2c1] | (R)-2-((1H-pyrrolo[2,3-b]pyridin-5- yl)oxy)-4-(4-(1-bromo-6,7,8,9- tetrahydro-5H-benzo[7]annulen-5- yl)piperazin-1-y1)-N-((3-nitro-4- (((tetrahydro-2H-pyran-4- yl)methyl)amino)phenyl)sulfonyl)benz- amide |
| 18B | 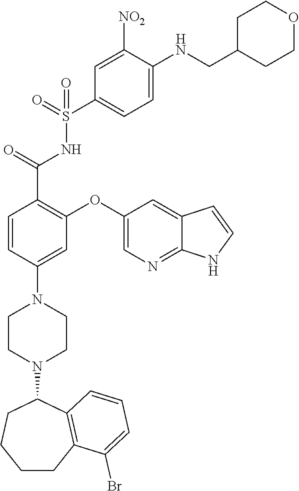 [SMILES: O=C(NS(=O)(=O)c1ccc(NCC2CCOCC2)c([N+](=O)[O-])c1)c1ccc(N2CCN([C@H]3CCCCc4c(Br)cccc43)CC2)cc1Oc1cnc2[nH]ccc2c1] | (S)-2-((1H-pyrrolo[2,3-b]pyridin-5- yl)oxy)-4-(4-(1-bromo-6,7,8,9- tetrahydro-5H-benzo[7]annulen-5- yl)piperazin-1-y1)-N-((3-nitro-4- (((tetrahydro-2H-pyran-4- yl)methyl)amino)phenyl)sulfonyl)benz- amide |
| 19 | 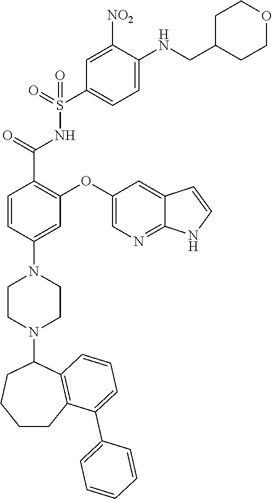 [SMILES: O=C(NS(=O)(=O)c1ccc(NCC2CCOCC2)c([N+](=O)[O-])c1)c1ccc(N2CCN(C3CCCCc4c(-c5ccccc5)cccc43)CC2)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(1-phenyl-6,7,8,9-tetrahydro-5H- benzo[7]annulen-5-yl)piperazin-1- yl)benzamide |
| 20 | 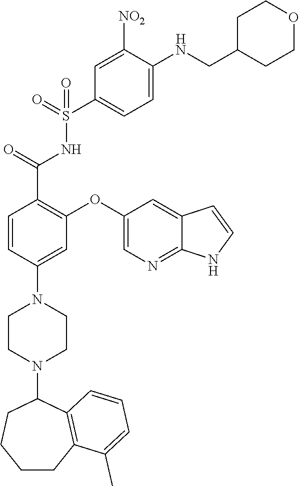 [SMILES: Cc1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1] | 2-((1H-pyrrolo[2,3-b]pyridin-5- yl)oxy)-4-(4-(1-methyl-6,7,8,9- tetrahydro-5H-benzo[7]annulen-5- yl)piperazin-1-y1)-N-((3-nitro-4- (((tetrahydro-2H-pyran-4- yl)methyl)amino)phenyl)sulfonyl)benz- amide |
| 21 | 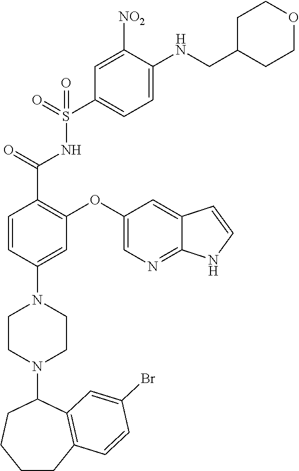 [SMILES: O=C(NS(=O)(=O)c1ccc(NCC2CCOCC2)c([N+](=O)[O-])c1)c1ccc(N2CCN(C3CCCCc4ccc(Br)cc43)CC2)cc1Oc1cnc2[nH]ccc2c1] | 2-((1H-pyrrolo[2,3-b]pyridin-5- yl)oxy)-4-(4-(3-bromo-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 22 | 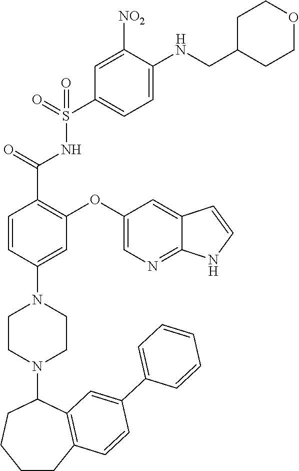 [SMILES: O=C(NS(=O)(=O)c1ccc(NCC2CCOCC2)c([N+](=O)[O-])c1)c1ccc(N2CCN(C3CCCCc4ccc(-c5ccccc5)cc43)CC2)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(3-phenyl-6,7,8,9-tetrahydro-5H- benzo[7]annulen-5-yl)piperazin-1- yl)benzamide |
| 23 | 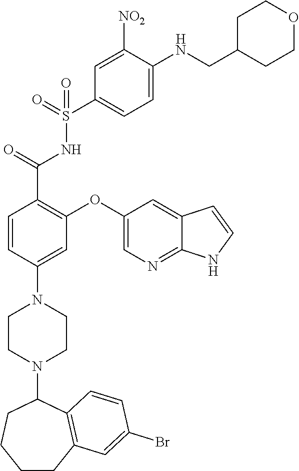 [SMILES: O=C(NS(=O)(=O)c1ccc(NCC2CCOCC2)c([N+](=O)[O-])c1)c1ccc(N2CCN(C3CCCCc4cc(Br)ccc43)CC2)cc1Oc1cnc2[nH]ccc2c1] | 2-((1H-pyrrolo[2,3-b]pyridin-5- yl)oxy)-4-(4-(2-bromo-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 24 | 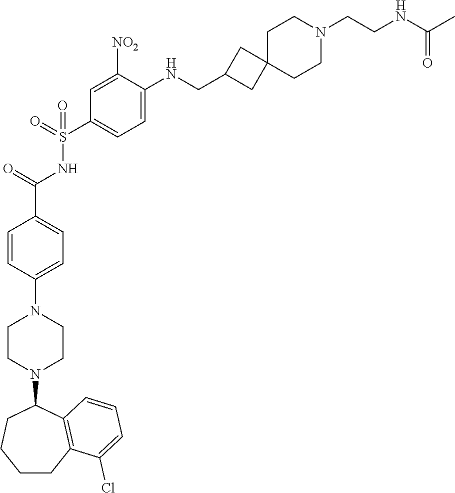 [SMILES: CC(=O)NCCN1CCC2(CC1)CC(CNc1ccc(S(=O)(=O)NC(=O)c3ccc(N4CCN([C@@H]5CCCCc6c(Cl)cccc65)CC4)cc3)cc1[N+](=O)[O-])C2] | (R)-N-((4-(((7-(2-acetamidoethyl)-7- azaspiro[3.5]nonan-2- yl)methyl)amino)-3- nitrophenyl)sulfony1)-4-(4-(1-chloro- 6,7,8,9-tetrahydro-5H- benzo[7]annulen-5-y1)piperazin-1- yl)benzamide |
| 25 | 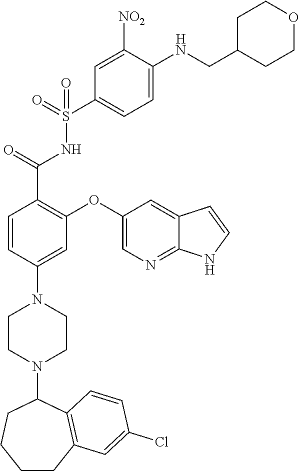 [SMILES: O=C(NS(=O)(=O)c1ccc(NCC2CCOCC2)c([N+](=O)[O-])c1)c1ccc(N2CCN(C3CCCCc4cc(Cl)ccc43)CC2)cc1Oc1cnc2[nH]ccc2c1] | 2-((1H-pyrrolo[2,3-b]pyridin-5- yl)oxy)-4-(4-(2-chloro-6,7,8,9- tetrahydro-5H-benzo[7]annulen-5- yl)piperazin-1-y1)-N-((3-nitro-4- (((tetrahydro-2H-pyran-4- yl)methyl)amino)phenyl)sulfonyl)benz- amide |
| 26 | 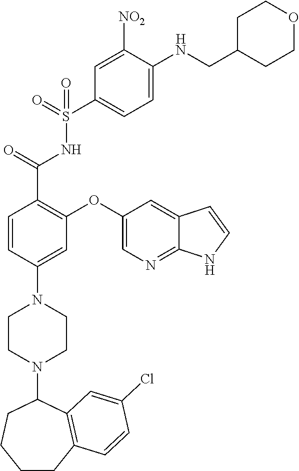 [SMILES: O=C(NS(=O)(=O)c1ccc(NCC2CCOCC2)c([N+](=O)[O-])c1)c1ccc(N2CCN(C3CCCCc4ccc(Cl)cc43)CC2)cc1Oc1cnc2[nH]ccc2c1] | 2-((1H-pyrrolo[2,3-b]pyridin-5- yl)oxy)-4-(4-(3-chloro-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 27 | 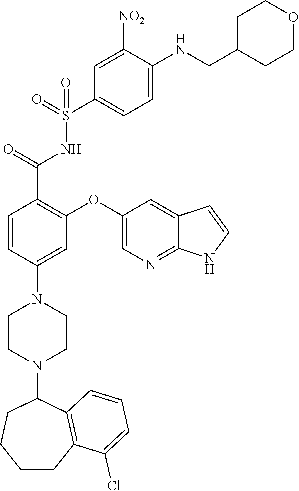 [SMILES: O=C(NS(=O)(=O)c1ccc(NCC2CCOCC2)c([N+](=O)[O-])c1)c1ccc(N2CCN(C3CCCCc4c(Cl)cccc43)CC2)cc1Oc1cnc2[nH]ccc2c1] | 2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 27A | 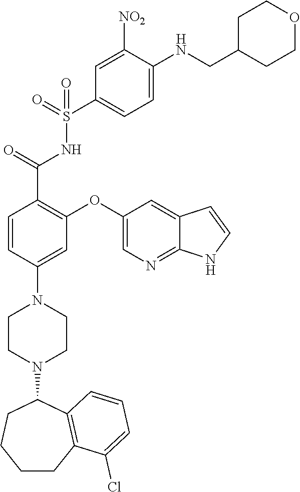 [SMILES: O=C(NS(=O)(=O)c1ccc(NCC2CCOCC2)c([N+](=O)[O-])c1)c1ccc(N2CCN([C@H]3CCCCc4c(Cl)cccc43)CC2)cc1Oc1cnc2[nH]ccc2c1] | (S)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 27B | 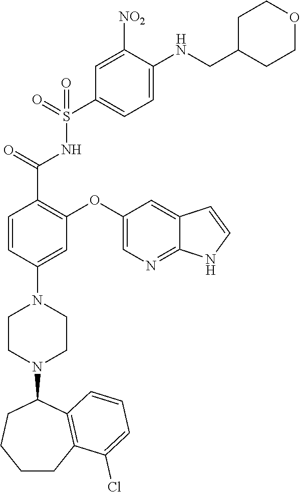 [SMILES: O=C(NS(=O)(=O)c1ccc(NCC2CCOCC2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 28 | 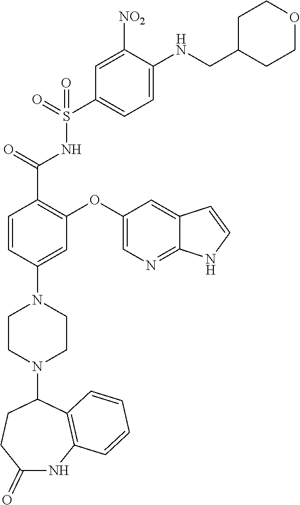 [SMILES: O=C1CCC(N2CCN(c3ccc(C(=O)NS(=O)(=O)c4ccc(NCC5CCOCC5)c([N+](=O)[O-])c4)c(Oc4cnc5[nH]ccc5c4)c3)CC2)c2ccccc2N1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(2-oxo-2,3,4,5-tetrahydro-1H- benzo[b]azepin-5-yl)piperazin-1- yl)benzamide |
| 29 | 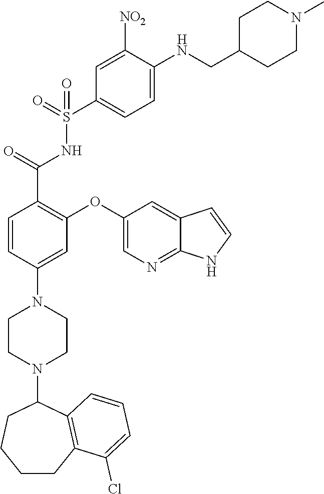 [SMILES: CN1CCC(CNc2ccc(S(=O)(=O)NC(=O)c3ccc(N4CCN(C5CCCCc6c(Cl)cccc65)CC4)cc3Oc3cnc4[nH]ccc4c3)cc2[N+](=O)[O-])CC1] | 2-((1H-pyrrolo[2,3-b]pyridin-5- yl)oxy)-4-(4-(1-chloro-6,7,8,9- tetrahydro-5H-benzo[7]annulen-5- yl)piperazin-1-y1)-N-((4-(((1- methylpiperidin-4-y1)methyl)amino)- 3-nitrophenyl)sulfonyl)benzamide |
| 30 | 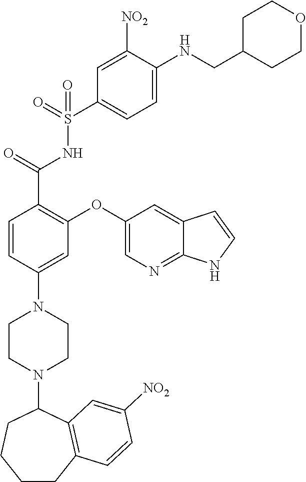 [SMILES: O=C(NS(=O)(=O)c1ccc(NCC2CCOCC2)c([N+](=O)[O-])c1)c1ccc(N2CCN(C3CCCCc4ccc([N+](=O)[O-])cc43)CC2)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(3-nitro-6,7,8,9-tetrahydro-5H- benzo[7]annulen-5-yl)piperazin-1- yl)benzamide |
| 31 | 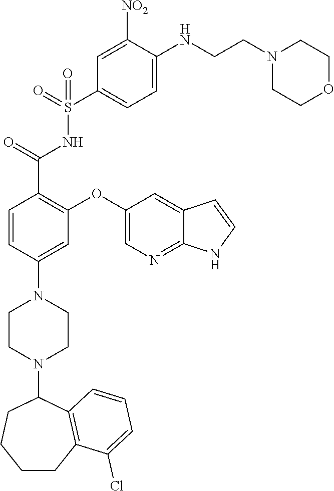 [SMILES: O=C(NS(=O)(=O)c1ccc(NCCN2CCOCC2)c([N+](=O)[O-])c1)c1ccc(N2CCN(C3CCCCc4c(Cl)cccc43)CC2)cc1Oc1cnc2[nH]ccc2c1] | 2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((4-((2- morpholinoethyl)amino)-3- nitrophenyl)sulfonyl)benzamide |
| 31A | 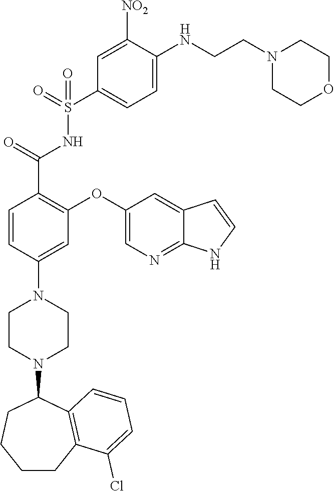 [SMILES: O=C(NS(=O)(=O)c1ccc(NCCN2CCOCC2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-y1)-N-((4-((2- morpholinoethyl)amino)-3- nitrophenyl)sulfonyl)benzamide |
| 31B | 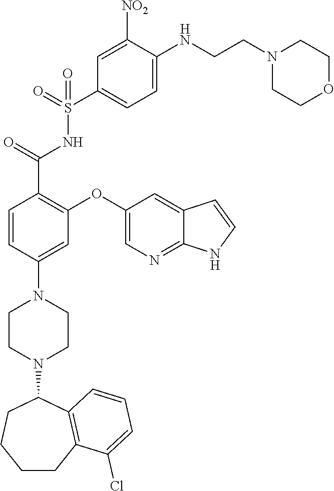 [SMILES: O=C(NS(=O)(=O)c1ccc(NCCN2CCOCC2)c([N+](=O)[O-])c1)c1ccc(N2CCN([C@H]3CCCCc4c(Cl)cccc43)CC2)cc1Oc1cnc2[nH]ccc2c1] | (S)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((4-((2- morpholinoethyl)amino)-3- nitrophenyl)sulfonyl)benzamide |
| 32 | 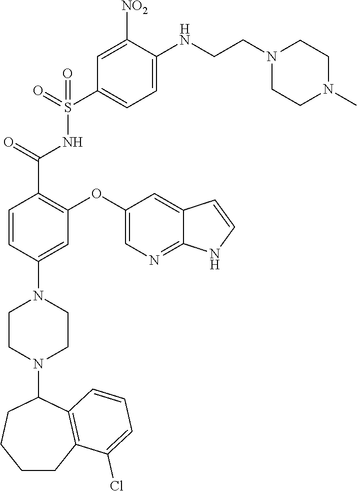 [SMILES: CN1CCN(CCNc2ccc(S(=O)(=O)NC(=O)c3ccc(N4CCN(C5CCCCc6c(Cl)cccc65)CC4)cc3Oc3cnc4[nH]ccc4c3)cc2[N+](=O)[O-])CC1] | 2-((1H-pyrrolo[2,3-b]pyridin-5- yl)oxy)-4-(4-(1-chloro-6,7,8,9- tetrahydro-5H-benzo[7]annulen-5- yl)piperazin-1-y1)-N-((4-((2-(4- methylpiperazin-1-yl)ethyl)amino)-3- nitrophenyl)sulfonyl)benzamide |
| 33 | 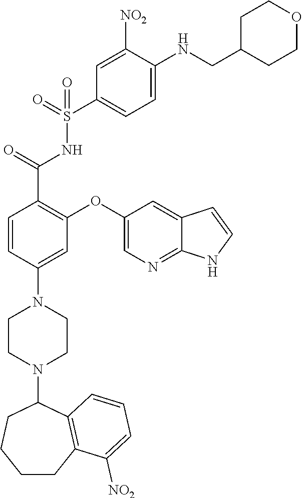 [SMILES: O=C(NS(=O)(=O)c1ccc(NCC2CCOCC2)c([N+](=O)[O-])c1)c1ccc(N2CCN(C3CCCCc4c3cccc4[N+](=O)[O-])CC2)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(1-nitro-6,7,8,9-tetrahydro-5H- benzo[7]annulen-5-y1)piperazin-1- yl)benzamide |
| 34 | 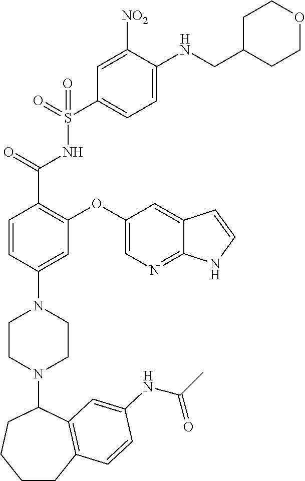 [SMILES: CC(=O)Nc1ccc2c(c1)C(N1CCN(c3ccc(C(=O)NS(=O)(=O)c4ccc(NCC5CCOCC5)c([N+](=O)[O-])c4)c(Oc4cnc5[nH]ccc5c4)c3)CC1)CCCC2] | 2-((1H-pyrrolo[2,3-b]pyridin-5- yl)oxy)-4-(4-(3-acetamido-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 35 | 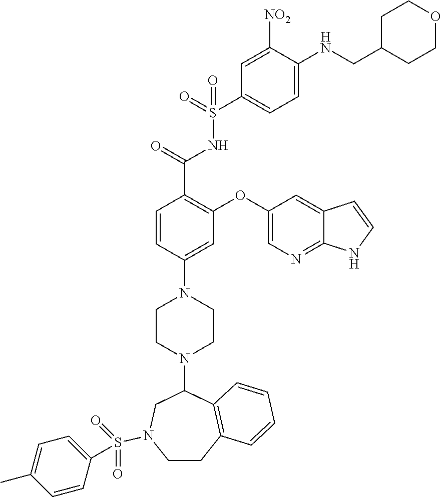 [SMILES: Cc1ccc(S(=O)(=O)N2CCc3ccccc3C(N3CCN(c4ccc(C(=O)NS(=O)(=O)c5ccc(NCC6CCOCC6)c([N+](=O)[O-])c5)c(Oc5cnc6[nH]ccc6c5)c4)CC3)C2)cc1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(3-tosyl-2,3,4,5-tetrahydro-1H- benzo[d]azepin-1-yl)piperazin-1- yl)benzamide |
| 36 | 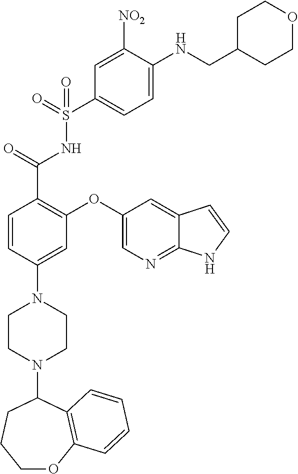 [SMILES: O=C(NS(=O)(=O)c1ccc(NCC2CCOCC2)c([N+](=O)[O-])c1)c1ccc(N2CCN(C3CCCOc4ccccc43)CC2)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(2,3,4,5-tetrahydrobenzo[b]oxepin- 5-y1)piperazin-1-yl)benzamide |
| 37 | 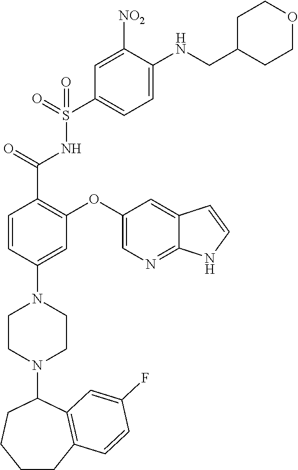 [SMILES: O=C(NS(=O)(=O)c1ccc(NCC2CCOCC2)c([N+](=O)[O-])c1)c1ccc(N2CCN(C3CCCCc4ccc(F)cc43)CC2)cc1Oc1cnc2[nH]ccc2c1] | 2-((1H-pyrrolo[2,3-b]pyridin-5- yl)oxy)-4-(4-(3-fluoro-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 38 | 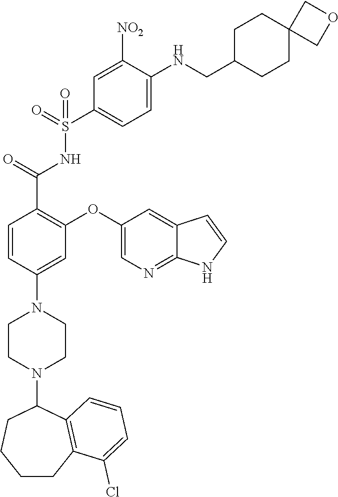 [SMILES: O=C(NS(=O)(=O)c1ccc(NCC2CCC3(CC2)COC3)c([N+](=O)[O-])c1)c1ccc(N2CCN(C3CCCCc4c(Cl)cccc43)CC2)cc1Oc1cnc2[nH]ccc2c1] | 2-((1H-pyrrolo[2,3-b]pyridin-5- yl)oxy)-N-((4-(((2- oxaspiro[3.5]nonan-7- yl)methyl)amino)-3- nitrophenyl)sulfonyl)-4-(4-(1-chloro- 6,7,8,9-tetrahydro-5H- benzo[7]annulen-5-yl)piperazin-1- yl)benzamide |
| 39 | 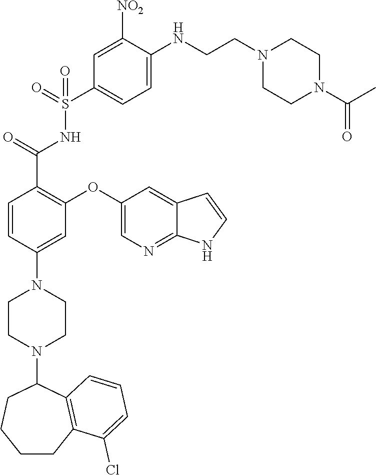 [SMILES: CC(=O)N1CCN(CCNc2ccc(S(=O)(=O)NC(=O)c3ccc(N4CCN(C5CCCCc6c(Cl)cccc65)CC4)cc3Oc3cnc4[nH]ccc4c3)cc2[N+](=O)[O-])CC1] | 2-((1H-pyrrolo[2,3-b]pyridin-5- yl)oxy)-N-((4-((2-(4-acetylpiperazin- 1-yl)ethyl)amino)-3- nitrophenyl)sulfony1)-4-(4-(1-chloro- 6,7,8,9-tetrahydro-5H- benzo[7]annulen-5-y1)piperazin-1- yl)benzamide |
| 40 | 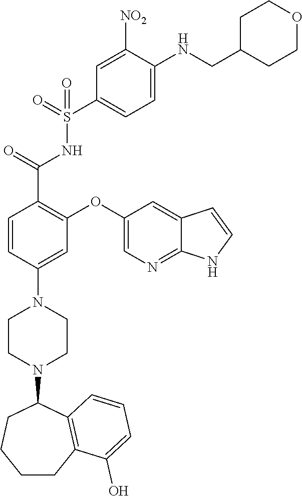 [SMILES: O=C(NS(=O)(=O)c1ccc(NCC2CCOCC2)c([N+](=O)[O-])c1)c1ccc(N2CCN([C@@H]3CCCCc4c(O)cccc43)CC2)cc1Oc1cnc2[nH]ccc2c1] | (R)-2-((1H-pyrrolo[2,3-b]pyridin-5- yl)oxy)-4-(4-(1-hydroxy-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 41 | 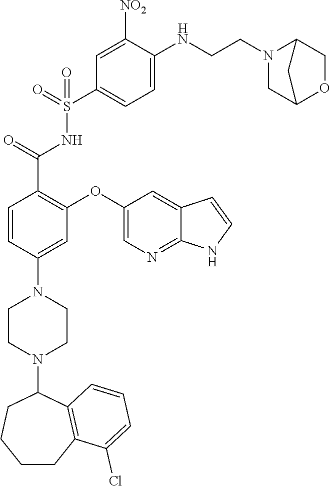 [SMILES: O=C(NS(=O)(=O)c1ccc(NCCN2CC3CC2CO3)c([N+](=O)[O-])c1)c1ccc(N2CCN(C3CCCCc4c(Cl)cccc43)CC2)cc1Oc1cnc2[nH]ccc2c1] | 2-((1H-pyrrolo[2,3-b]pyridin-5- yl)oxy)-N-((4-((2-(2-oxa-5- azabicyclo[2.2.1 ]heptan-5- yl)ethyl)amino)-3- nitrophenyl)sulfonyl)-4-(4-(1-chloro- 6,7,8,9-tetrahydro-5H- benzo[7]annulen-5-yl)piperazin-1- yl)benzamide |
| 42 | 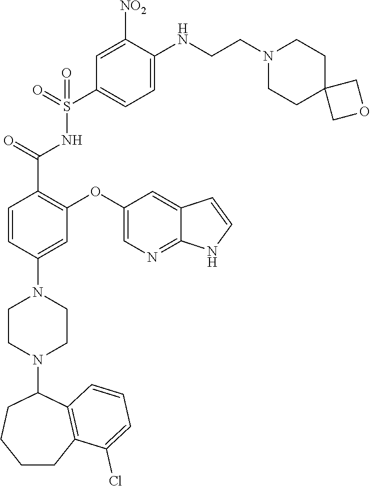 [SMILES: O=C(NS(=O)(=O)c1ccc(NCCN2CCC3(CC2)COC3)c([N+](=O)[O-])c1)c1ccc(N2CCN(C3CCCCc4c(Cl)cccc43)CC2)cc1Oc1cnc2[nH]ccc2c1] | 2-((1H-pyrrolo[2,3-b]pyridin-5- yl)oxy)-N-((4-((2-(2-oxa-7- azaspiro[3.5]nonan-7-yl)ethyl)amino)- 3-nitrophenyl)sulfonyl)-4-(4-(1- chloro-6,7,8,9-tetrahydro-5H- benzo[7]annulen-5-yl)piperazin-1- yl)benzamide |
| 43 | 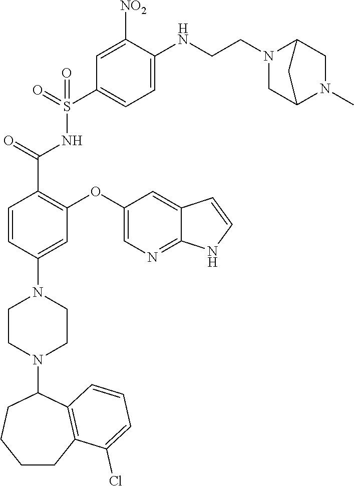 [SMILES: CN1CC2CC1CN2CCNc1ccc(S(=O)(=O)NC(=O)c2ccc(N3CCN(C4CCCCc5c(Cl)cccc54)CC3)cc2Oc2cnc3[nH]ccc3c2)cc1[N+](=O)[O-]] | 2-((1H-pyrrolo[2,3-b]pyridin-5- yl)oxy)-4-(4-(1-chloro-6,7,8,9- tetrahydro-5H-benzo[7]annulen-5- yl)piperazin-1-y1)-N-((4-((2-(5- methyl-2,5-diazabicyclo[2.2.1]heptan- 2-yl)ethyl)amino)-3- nitrophenyl)sulfonyl)benzamide |
| 44 | 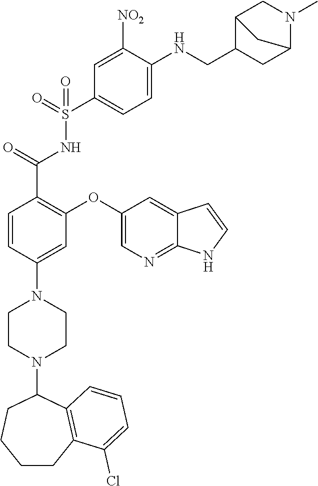 [SMILES: CN1CC2CC1CC2CNc1ccc(S(=O)(=O)NC(=O)c2ccc(N3CCN(C4CCCCc5c(Cl)cccc54)CC3)cc2Oc2cnc3[nH]ccc3c2)cc1[N+](=O)[O-]] | 2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((4-(((2-methyl- 2-azabicyclo[2.2.1]heptan-5- yl)methyl)amino)-3- nitrophenyl)sulfonyl)benzamide |
| 45 | 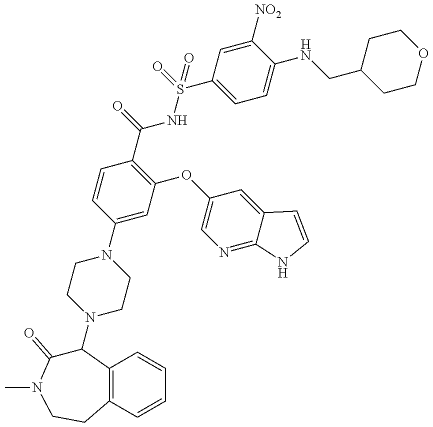 [SMILES: CN1CCc2ccccc2C(N2CCN(c3ccc(C(=O)NS(=O)(=O)c4ccc(NCC5CCOCC5)c([N+](=O)[O-])c4)c(Oc4cnc5[nH]ccc5c4)c3)CC2)C1=O] | 2-((1H-pyrrolo[2,3-b]pyridin-5- yl)oxy)-4-(4-(3-methyl-2-oxo-2,3,4,5- tetrahydro-1H-benzo[d]azepin-1- yl)piperazin-1-yl)-N-((3-nitro-4- (((tetrahydro-2H-pyran-4- yl)methyl)amino)phenyl)sulfonyl)benz- amide |
| 46 | 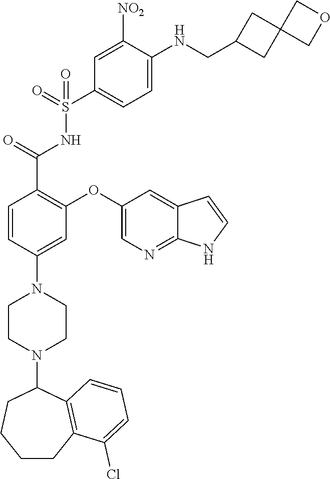 [SMILES: O=C(NS(=O)(=O)c1ccc(NCC2CC3(COC3)C2)c([N+](=O)[O-])c1)c1ccc(N2CCN(C3CCCCc4c(Cl)cccc43)CC2)cc1Oc1cnc2[nH]ccc2c1] | 2-((1H-pyrrolo[2,3-b]pyridin-5- yl)oxy)-N-((4-(((2- oxaspiro[3.3]heptan-6- yl)methyl)amino)-3- nitrophenyl)sulfonyl)-4-(4-(1-chloro- 6,7,8,9-tetrahydro-5H- benzo[7]annulen-5-yl)piperazin-1- yl)benzamide |
| 47 | 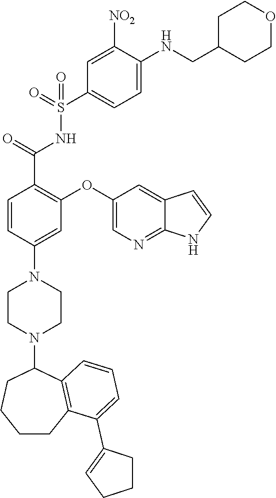 [SMILES: O=C(NS(=O)(=O)c1ccc(NCC2CCOCC2)c([N+](=O)[O-])c1)c1ccc(N2CCN(C3CCCCc4c(C5=CCCC5)cccc43)CC2)cc1Oc1cnc2[nH]ccc2c1] | 2-((1H-pyrrolo[2,3-b]pyridin-5- yl)oxy)-4-(4-(1-(cyclopent-1-en-1-yl)- 6,7,8,9-tetrahydro-5H- benzo[7]annulen-5-yl)piperazin-1-yl)- N-((3-nitro-4-(((tetrahydro-2H-pyran- 4- yl)methyl)amino)phenyl)sulfonyl)benz- amide |
| 48 | 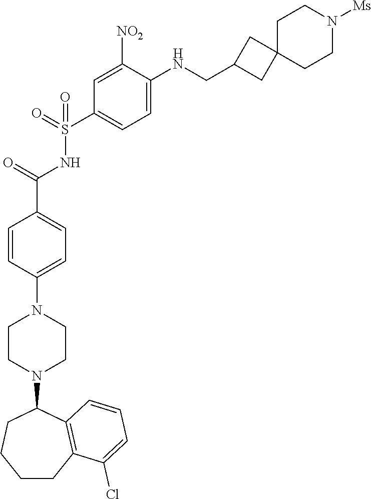 [SMILES: CS(=O)(=O)N1CCC2(CC1)CC(CNc1ccc(S(=O)(=O)NC(=O)c3ccc(N4CCN([C@@H]5CCCCc6c(Cl)cccc65)CC4)cc3)cc1[N+](=O)[O-])C2] | (R)-4-(4-(1-chloro-6,7,8,9-tetrahydro- 5H-benzo[7]annulen-5-yl)piperazin-1- yl)-N-((4-(((7-(methylsulfonyl)-7- azaspiro[3.5]nonan-2- yl)methyl)amino)-3- nitrophenyl)sulfony1)benzamide |
| 49 | 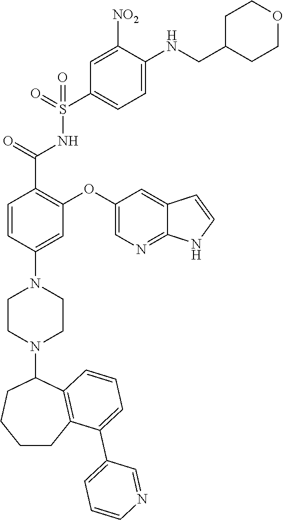 [SMILES: O=C(NS(=O)(=O)c1ccc(NCC2CCOCC2)c([N+](=O)[O-])c1)c1ccc(N2CCN(C3CCCCc4c(-c5cccnc5)cccc43)CC2)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(1-(pyridin-3-yl)-6,7,8,9- tetrahydro-5H-benzo[7]annulen-5- yl)piperazin-1-y1)benzamide |
| 50 | 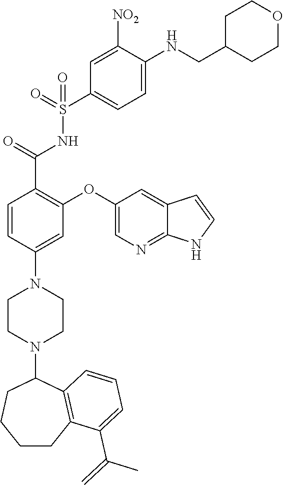 [SMILES: C=C(C)c1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(1-(prop-1-en-2-y1)-6,7,8,9- tetrahydro-5H-benzo[7]annulen-5- yl)piperazin-1-yl)benzamide |
| 51 | 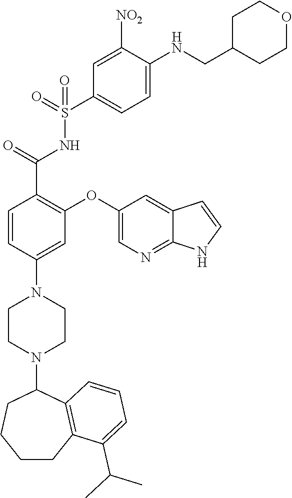 [SMILES: CC(C)c1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1] | 2-((1H-pyrrolo[2,3-b]pyridin-5- yl)oxy)-4-(4-(1-isopropyl-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 52 | 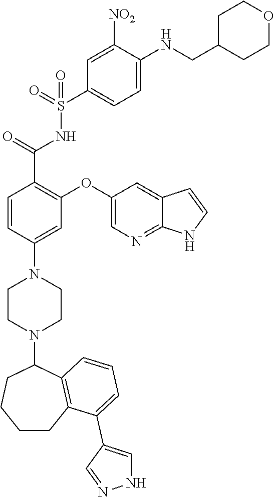 [SMILES: O=C(NS(=O)(=O)c1ccc(NCC2CCOCC2)c([N+](=O)[O-])c1)c1ccc(N2CCN(C3CCCCc4c(-c5cn[nH]c5)cccc43)CC2)cc1Oc1cnc2[nH]ccc2c1] | 4-(4-(1-(1H-pyrazol-4-y1)-6,7,8,9- tetrahydro-5H-benzo[7]annulen-5- yl)piperazin-1-y1)-2-((1H-pyrrolo[2,3- b]pyridin-5-yl)oxy)-N-((3-nitro-4- (((tetrahydro-2H-pyran-4- yl)methyl)amino)phenyl)sulfonyl)benz- amide |
| 53 | 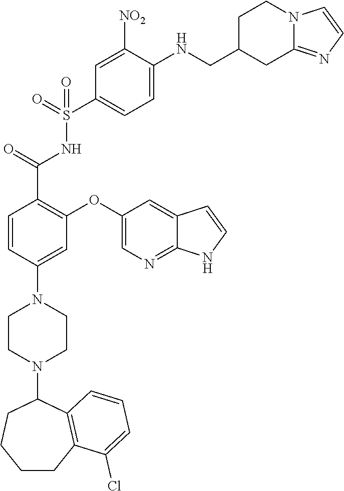 [SMILES: O=C(NS(=O)(=O)c1ccc(NCC2CCn3ccnc3C2)c([N+](=O)[O-])c1)c1ccc(N2CCN(C3CCCCc4c(Cl)cccc43)CC2)cc1Oc1cnc2[nH]ccc2c1] | 2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((3-nitro-4- (((5,6,7,8-tetrahydroimidazo[1,2- a]pyridin-7- yl)methyl)amino)phenyl)sulfonyl)benz- amide |
| 54 | 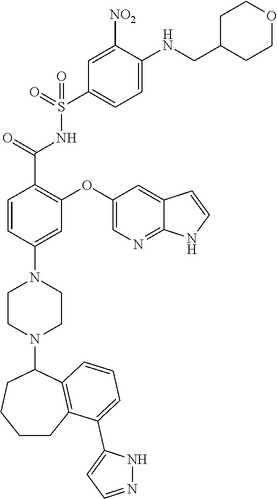 [SMILES: O=C(NS(=O)(=O)c1ccc(NCC2CCOCC2)c([N+](=O)[O-])c1)c1ccc(N2CCN(C3CCCCc4c(-c5ccn[nH]5)cccc43)CC2)cc1Oc1cnc2[nH]ccc2c1] | 4-(4-(1-(1H-pyrazol-3-y1)-6,7,8,9- tetrahydro-5H-benzo[7]annulen-5- yl)piperazin-1-yl)-2-((1H-pyrrolo[2,3- b]pyridin-5-yl)oxy)-N-((3-nitro-4- (((tetrahydro-2H-pyran-4- yl)methyl)amino)phenyl)sulfonyl)benz- amide |
| 55 | 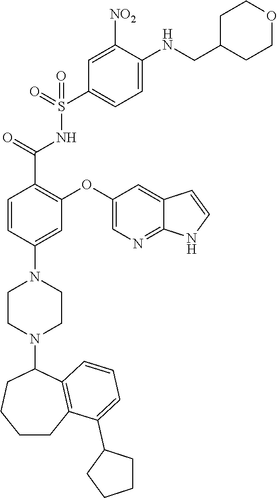 [SMILES: O=C(NS(=O)(=O)c1ccc(NCC2CCOCC2)c([N+](=O)[O-])c1)c1ccc(N2CCN(C3CCCCc4c(C5CCCC5)cccc43)CC2)cc1Oc1cnc2[nH]ccc2c1] | 2-((1H-pyrrolo[2,3-b]pyridin-5- yl)oxy)-4-(4-(1-cyclopentyl-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 56 | 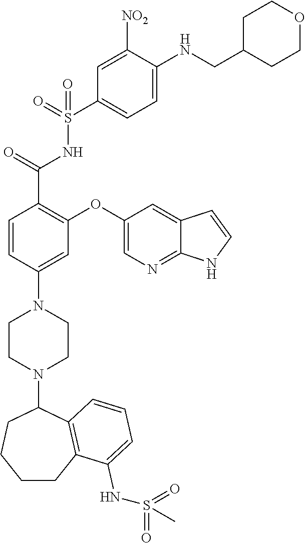 [SMILES: CS(=O)(=O)Nc1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1] | 2-((1H-pyrrolo[2,3-b]pyridin-5- yl)oxy)-4-(4-(1-(methylsulfonamido)- 6,7,8,9-tetrahydro-5H- benzo[7]annulen-5-y1)piperazin-1-yl)- N-((3-nitro-4-(((tetrahydro-2H-pyran- 4- yl)methyl)amino)phenyl)sulfonyl)benz- amide |
| 57 | 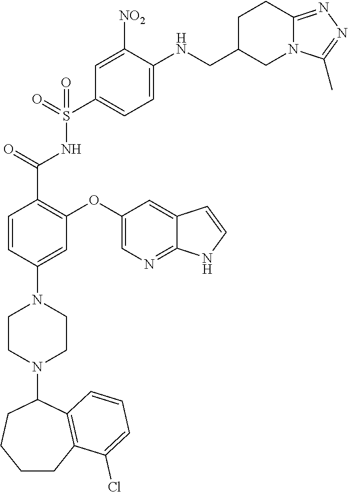 [SMILES: Cc1nnc2n1CC(CNc1ccc(S(=O)(=O)NC(=O)c3ccc(N4CCN(C5CCCCc6c(Cl)cccc65)CC4)cc3Oc3cnc4[nH]ccc4c3)cc1[N+](=O)[O-])CC2] | 2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((4-(((3-methyl- 5,6,7,8-tetrahydro-[1,2,4]triazolo[4,3- a]pyridin-6-yl)methyl)amino)-3- nitrophenyl)sulfonyl)benzamide |
| 58 | 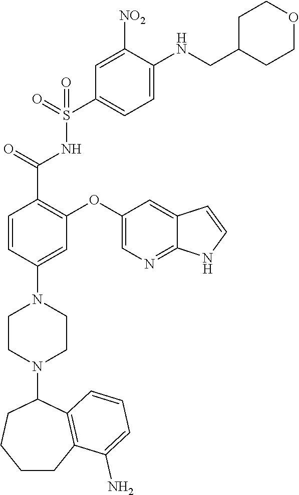 [SMILES: Nc1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1] | 2-((1H-pyrrolo[2,3-b]pyridin-5- yl)oxy)-4-(4-(1-amino-6,7,8,9- tetrahydro-5H-benzo[7]annulen-5- yl)piperazin-1-y1)-N-((3-nitro-4- (((tetrahydro-2H-pyran-4- yl)methyl)amino)phenyl)sulfonyl)benz- amide |
| 59 | 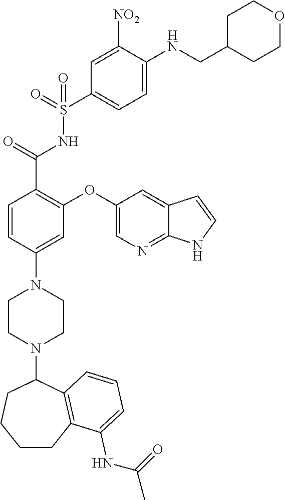 [SMILES: CC(=O)Nc1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1] | 2-((1H-pyrrolo[2,3-b]pyridin-5- yl)oxy)-4-(4-(1-acetamido-6,7,8,9- tetrahydro-5H-benzo[7]annulen-5- yl)piperazin-1-y1)-N-((3-nitro-4- (((tetrahydro-2H-pyran-4- yl)methyl)amino)phenyl)sulfonyl)benz- amide |
| 60 | 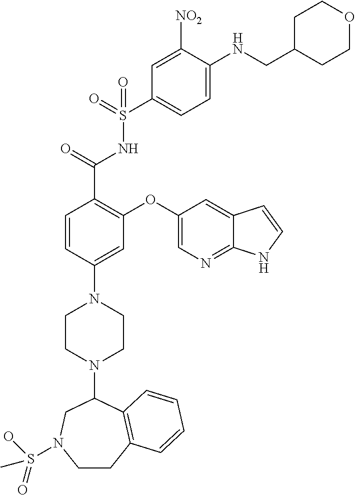 | 2-((1H-pyrrolo[2,3-b]pyridin-5- yl)oxy)-4-(4-(3-(methylsulfonyl)- 2,3,4,5-tetrahydro-1H-benzo[d]azepin- 1-yl)piperazin-1-yl)-N-((3-nitro-4- (((tetrahydro-2H-pyran-4- yl)methyl)amino)phenyl)sulfonyl)benz- amide |
| 61 | 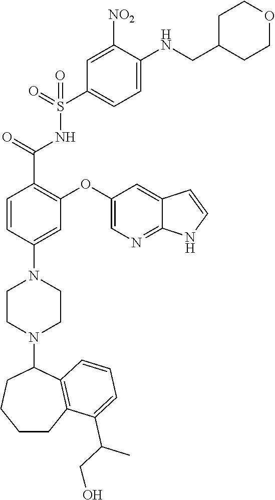 [SMILES: CC(CO)c1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1] | 2-((1H-pyrrolo[2,3-b]pyridin-5- yl)oxy)-4-(4-(1-(1-hydroxypropan-2- yl)-6,7,8,9-tetrahydro-5H- benzo[7]annulen-5-y1)piperazin-1-y1)- N-((3-nitro-4-(((tetrahydro-2H-pyran- 4- yl)methyl)amino)phenyl)sulfonyl)benz- amide |
| 62 | 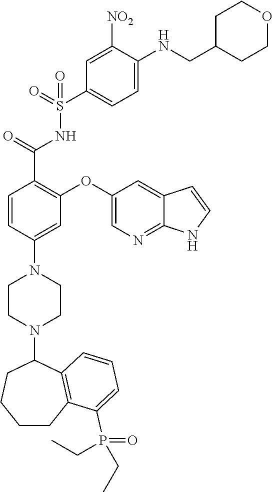 [SMILES: CCP(=O)(CC)c1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1] | 2-((1H-pyrrolo[2,3-b]pyridin-5- yl)oxy)-4-(4-(1-(diethylphosphoryl)- 6,7,8,9-tetrahydro-5H- benzo[7]annulen-5-y1)piperazin-1-yl)- N-((3-nitro-4-(((tetrahydro-2H-pyran- 4- yl)methyl)amino)phenyl)sulfonyl)benz- amide |
| 63 | 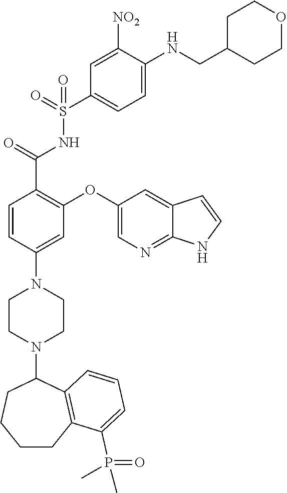 [SMILES: CP(C)(=O)c1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1] | 2-((1H-pyrrolo[2,3-b]pyridin-5- yl)oxy)-4-(4-(1-(dimethylphosphoryl)- 6,7,8,9-tetrahydro-5H- benzo[7]annulen-5-y1)piperazin-1-yl)- N-((3-nitro-4-(((tetrahydro-2H-pyran- 4- yl)methyl)amino)phenyl)sulfonyl)benz- amide |
| 64 | 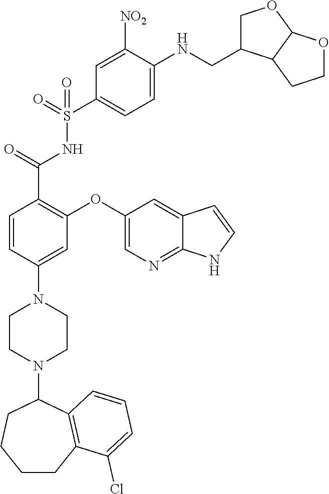 [SMILES: O=C(NS(=O)(=O)c1ccc(NCC2COC3OCCC23)c([N+](=O)[O-])c1)c1ccc(N2CCN(C3CCCCc4c(Cl)cccc43)CC2)cc1Oc1cnc2[nH]ccc2c1] | 2-((1H-pyrrolo[2,3-b]pyridin-5- yl)oxy)-4-(4-(1-chloro-6,7,8,9- tetrahydro-5H-benzo[7]annulen-5- yl)piperazin-1-y1)-N-((4- (((hexahydrofuro[2,3-b]furan-3- yl)methyl)amino)-3- nitrophenyl)sulfonyl)benzamide |
| 65 | 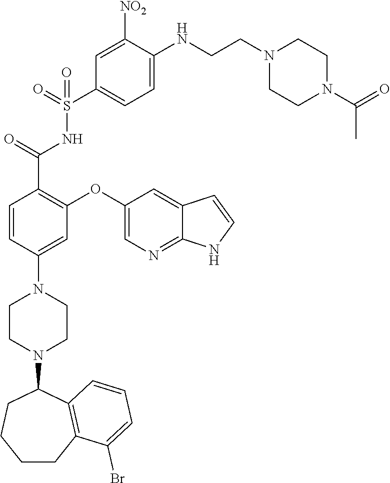 [SMILES: CC(=O)N1CCN(CCNc2ccc(S(=O)(=O)NC(=O)c3ccc(N4CCN([C@@H]5CCCCc6c(Br)cccc65)CC4)cc3Oc3cnc4[nH]ccc4c3)cc2[N+](=O)[O-])CC1] | (R)-2-((1H-pyrrolo[2,3-b]pyridin-5- yl)oxy)-N-((4-((2-(4-acetylpiperazin- 1-yl)ethyl)amino)-3- nitrophenyl)sulfonyl)-4-(4-(1-bromo- 6,7,8,9-tetrahydro-5H- benzo[7]annulen-5-yl)piperazin-1- yl)benzamide |
| 66 | 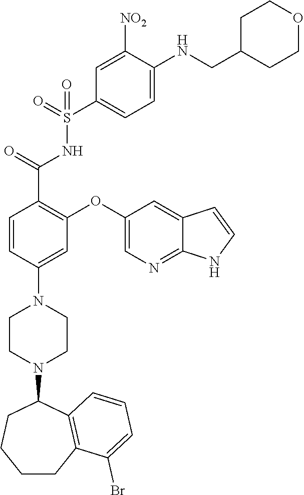 [SMILES: O=C(NS(=O)(=O)c1ccc(NCC2CCOCC2)c([N+](=O)[O-])c1)c1ccc(N2CCN([C@@H]3CCCCc4c(Br)cccc43)CC2)cc1Oc1cnc2[nH]ccc2c1] | (R)-2-((1H-pyrrolo[2,3-b]pyridin-5- yl)oxy)-4-(4-(1-bromo-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 67 | 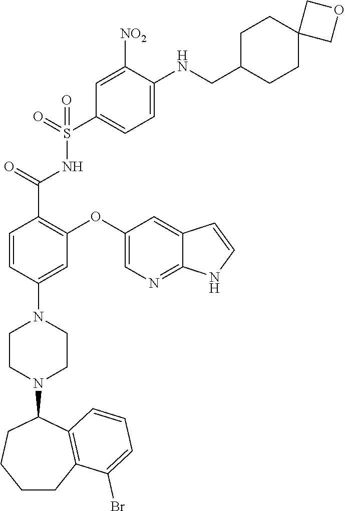 [SMILES: O=C(NS(=O)(=O)c1ccc(NCC2CCC3(CC2)COC3)c([N+](=O)[O-])c1)c1ccc(N2CCN([C@@H]3CCCCc4c(Br)cccc43)CC2)cc1Oc1cnc2[nH]ccc2c1] | (R)-2-((1H-pyrrolo[2,3-b]pyridin-5- yl)oxy)-N-((4-(((2- oxaspiro[3.5]nonan-7- yl)methyl)amino)-3- nitrophenyl)sulfonyl)-4-(4-(1-bromo- 6,7,8,9-tetrahydro-5H- benzo[7]annulen-5-yl)piperazin-1- yl)benzamide |
| 68 | 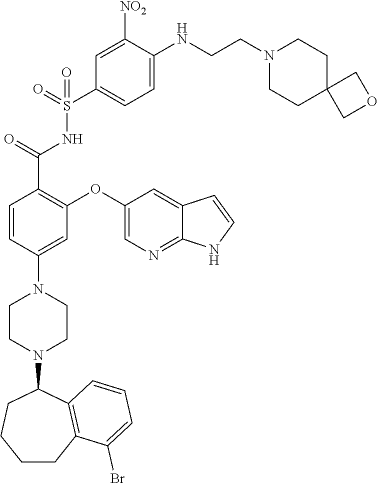 [SMILES: O=C(NS(=O)(=O)c1ccc(NCCN2CCC3(CC2)COC3)c([N+](=O)[O-])c1)c1ccc(N2CCN([C@@H]3CCCCc4c(Br)cccc43)CC2)cc1Oc1cnc2[nH]ccc2c1] | (R)-2-((1H-pyrrolo[2,3-b]pyridin-5- yl)oxy)-N-((4-((2-(2-oxa-7- azaspiro[3.5]nonan-7-yl)ethyl)amino)- 3-nitrophenyl)sulfonyl)-4-(4-(1- bromo-6,7,8,9-tetrahydro-5H- benzo[7]annulen-5-y1)piperazin-1- yl)benzamide |
| 69 | 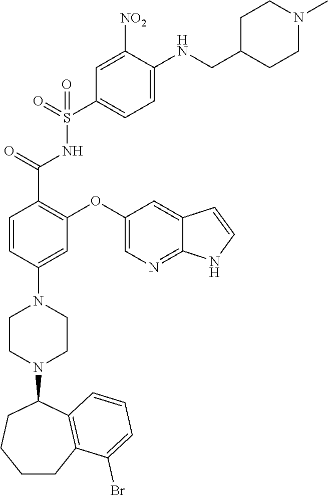 [SMILES: CN1CCC(CNc2ccc(S(=O)(=O)NC(=O)c3ccc(N4CCN([C@@H]5CCCCc6c(Br)cccc65)CC4)cc3Oc3cnc4[nH]ccc4c3)cc2[N+](=O)[O-])CC1] | (R)-2-((1H-pyrrolo[2,3-b]pyridin-5- yl)oxy)-4-(4-(1-bromo-6,7,8,9- tetrahydro-5H-benzo[7]annulen-5- yl)piperazin-1-yl)-N-((4-(((1- methylpiperidin-4-yl)methyl)amino)- 3-nitrophenyl)sulfonyl)benzamide |
| 70 | 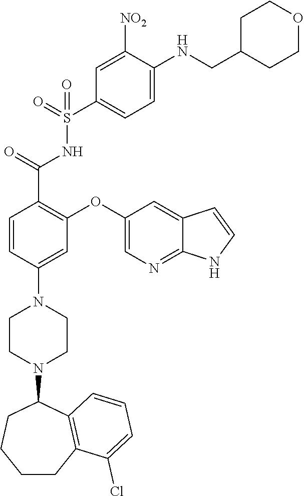 [SMILES: O=C(NS(=O)(=O)c1ccc(NCC2CCOCC2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 71 | 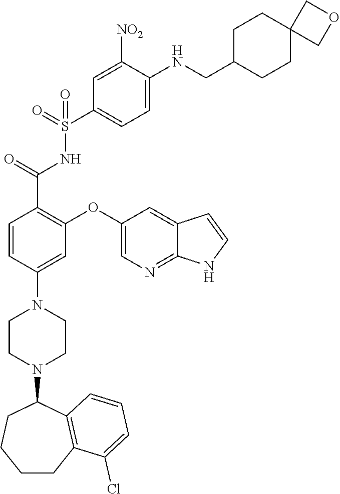 [SMILES: O=C(NS(=O)(=O)c1ccc(NCC2CCC3(CC2)COC3)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-N-((4-(((2- oxaspiro[3.5]nonan-7- yl)methyl)amino)-3- nitrophenyl)sulfonyl)-4-(4-(1-chloro- 6,7,8,9-tetrahydro-5H- benzo[7]annulen-5-yl)piperazin-1- yl)benzamide |
| 72 | 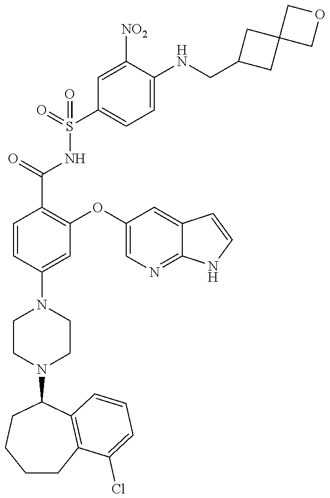 [SMILES: O=C(NS(=O)(=O)c1ccc(NCC2CC3(COC3)C2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-N-((4-(((2- oxaspiro[3.3]heptan-6- yl)methyl)amino)-3- nitrophenyl)sulfonyl)-4-(4-(1-chloro- 6,7,8,9-tetrahydro-5H- benzo[7]annulen-5-yl)piperazin-1- yl)benzamide |
| 73 | 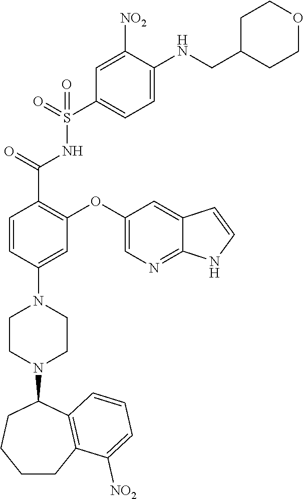 [SMILES: O=C(NS(=O)(=O)c1ccc(NCC2CCOCC2)c([N+](=O)[O-])c1)c1ccc(N2CCN([C@@H]3CCCCc4c3cccc4[N+](=O)[O-])CC2)cc1Oc1cnc2[nH]ccc2c1] | (R)-2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(1-nitro-6,7,8,9-tetrahydro-5H- benzo[7]annulen-5-y1)piperazin-1- yl)benzamide |
| 74 | 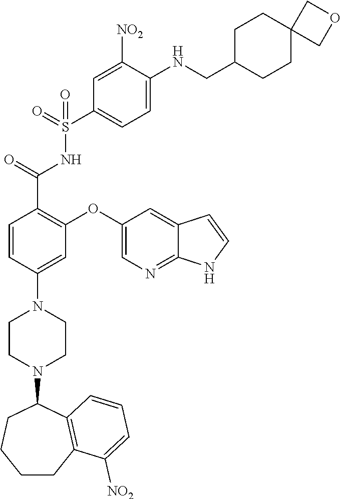 [SMILES: O=C(NS(=O)(=O)c1ccc(NCC2CCC3(CC2)COC3)c([N+](=O)[O-])c1)c1ccc(N2CCN([C@@H]3CCCCc4c3cccc4[N+](=O)[O-])CC2)cc1Oc1cnc2[nH]ccc2c1] | (R)-2-((1H-pyrrolo[2,3-b]pyridin-5- yl)oxy)-N-((4-(((2- oxaspiro[3.5]nonan-7- yl)methyl)amino)-3- nitrophenyl)sulfonyl)-4-(4-(1-nitro- 6,7,8,9-tetrahydro-5H- benzo[7]annulen-5-y1)piperazin-1- yl)benzamide |
| 75 | 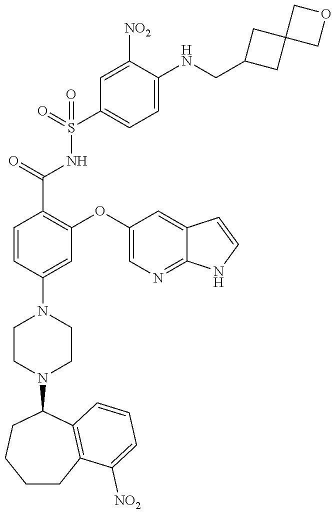 [SMILES: O=C(NS(=O)(=O)c1ccc(NCC2CC3(COC3)C2)c([N+](=O)[O-])c1)c1ccc(N2CCN([C@@H]3CCCCc4c3cccc4[N+](=O)[O-])CC2)cc1Oc1cnc2[nH]ccc2c1] | (R)-2-((1H-pyrrolo[2,3-b]pyridin-5- yl)oxy)-N-((4-(((2- oxaspiro[3.3]heptan-6- yl)methyl)amino)-3- nitrophenyl)sulfonyl)-4-(4-(1-nitro- 6,7,8,9-tetrahydro-5H- benzo[7]annulen-5-yl)piperazin-1- yl)benzamide |
| 76 | 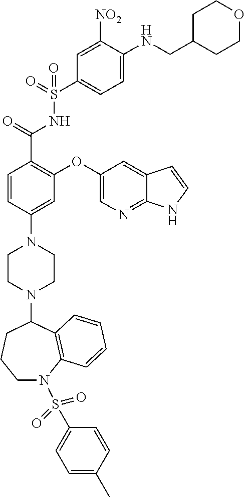 [SMILES: Cc1ccc(S(=O)(=O)N2CCCC(N3CCN(c4ccc(C(=O)NS(=O)(=O)c5ccc(NCC6CCOCC6)c([N+](=O)[O-])c5)c(Oc5cnc6[nH]ccc6c5)c4)CC3)c3ccccc32)cc1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (4-(1-tosy1-2,3,4,5-tetrahydro-1H- benzo[b]azepin-5-y1)piperazin-1- yl)benzamide |
| 77 | 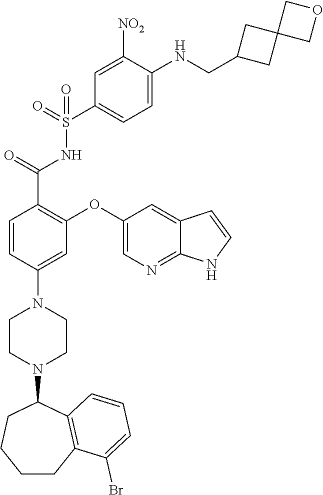 [SMILES: O=C(NS(=O)(=O)c1ccc(NCC2CC3(COC3)C2)c([N+](=O)[O-])c1)c1ccc(N2CCN([C@@H]3CCCCc4c(Br)cccc43)CC2)cc1Oc1cnc2[nH]ccc2c1] | (R)-2-((1H-pyrrolo[2,3-b]pyridin-5- yl)oxy)-N-((4-(((2- oxaspiro[3.3]heptan-6- yl)methyl)amino)-3- nitrophenyl)sulfony1)-4-(4-(1-bromo- 6,7,8,9-tetrahydro-5H- benzo[7]annulen-5-y1)piperazin-1- yl)benzamide |
| 78 | 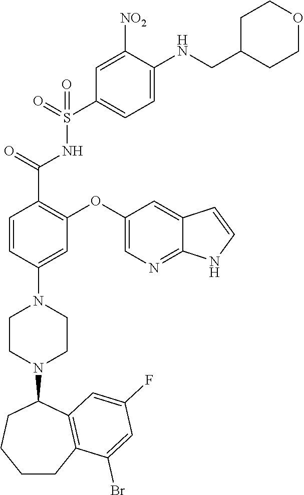 [SMILES: O=C(NS(=O)(=O)c1ccc(NCC2CCOCC2)c([N+](=O)[O-])c1)c1ccc(N2CCN([C@@H]3CCCCc4c(Br)cc(F)cc43)CC2)cc1Oc1cnc2[nH]ccc2c1] | (R)-2-((1H-pyrrolo[2,3-b]pyridin-5- yl)oxy)-4-(4-(1-bromo-3-fluoro- 6,7,8,9-tetrahydro-5H- benzo[7]annulen-5-yl)piperazin-1-yl)- N-((3-nitro-4-(((tetrahydro-2H-pyran- 4- yl)methyl)amino)phenyl)sulfonyl)benz- amide |
| 79 | 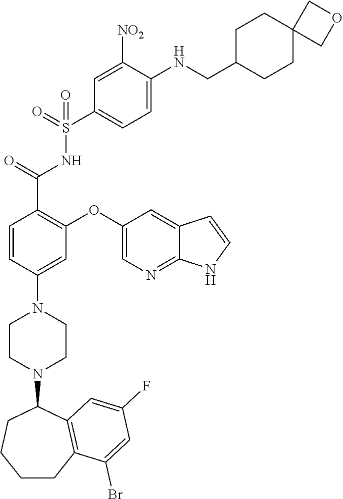 [SMILES: O=C(NS(=O)(=O)c1ccc(NCC2CCC3(CC2)COC3)c([N+](=O)[O-])c1)c1ccc(N2CCN([C@@H]3CCCCc4c(Br)cc(F)cc43)CC2)cc1Oc1cnc2[nH]ccc2c1] | (R)-2-((1H-pyrrolo[2,3-b]pyridin-5- yl)oxy)-N-((4-(((2- oxaspiro[3.5]nonan-7- yl)methyl)amino)-3- nitrophenyl)sulfonyl)-4-(4-(1-bromo- 3-fluoro-6,7,8,9-tetrahydro-5H- benzo[7]annulen-5-yl)piperazin-1- yl)benzamide |
| 80 | 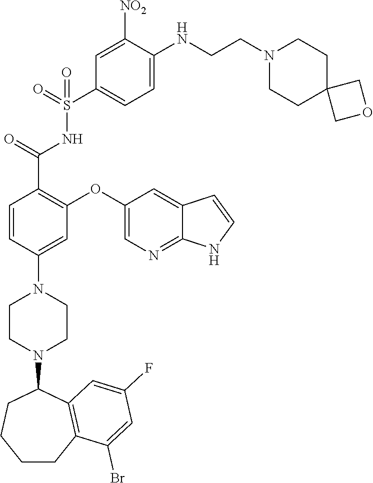 [SMILES: O=C(NS(=O)(=O)c1ccc(NCCN2CCC3(CC2)COC3)c([N+](=O)[O-])c1)c1ccc(N2CCN([C@@H]3CCCCc4c(Br)cc(F)cc43)CC2)cc1Oc1cnc2[nH]ccc2c1] | (R)-2-((1H-pyrrolo[2,3-b]pyridin-5- yl)oxy)-N-((4-((2-(2-oxa-7- azaspiro[3.5]nonan-7-yl)ethyl)amino)- 3-nitrophenyl)sulfony1)-4-(4-(1- bromo-3-fluoro-6,7,8,9-tetrahydro-5H- benzo[7]annulen-5-y1)piperazin-1- yl)benzamide |
| 81 | 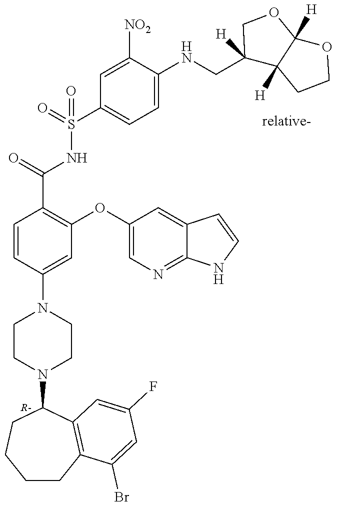 | 2-((1H-pyrrolo[2,3-b]pyridin-5- yl)oxy)-4-(4-((R)-1-bromo-3-fluoro- 6,7,8,9-tetrahydro-5H- benzo[7]annulen-5-y1)piperazin-1-yl)- N-((4-((((3R,3aR,6aS)- hexahydrofuro[2,3-b]furan-3- yl)methyl)amino)-3- nitrophenyl)sulfonyl)benzamide |
| 82 | 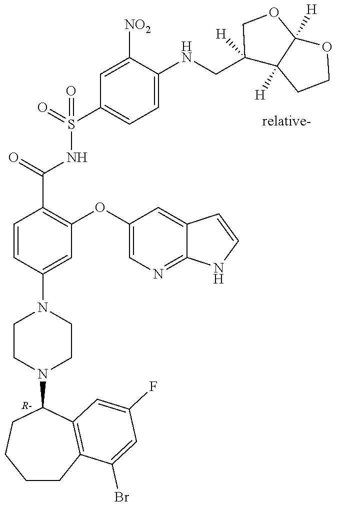 | 2-((1H-pyrrolo[2,3-b]pyridin-5- yl)oxy)-4-(4-((R)-1-bromo-3-fluoro- 6,7,8,9-tetrahydro-5H- benzo[7]annulen-5-yl)piperazin-1-yl)- N-((4-((((3S,3aS,6aR)- hexahydrofuro[2,3-b]furan-3- yl)methyl)amino)-3- nitrophenyl)sulfonyl)benzamide |
| 83 | 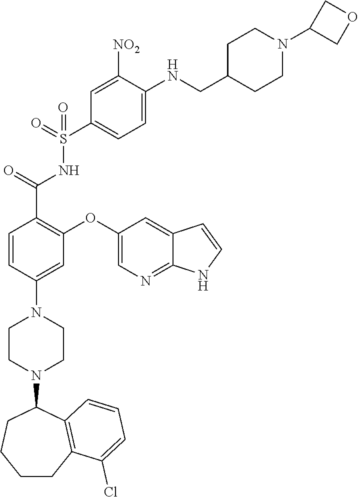 [SMILES: O=C(NS(=O)(=O)c1ccc(NCC2CCN(C3COC3)CC2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((3-nitro-4-(((1- (oxetan-3-y1)piperidin-4- yl)methyl)amino)phenyl)sulfonyl)benz- amide |
| 84 | 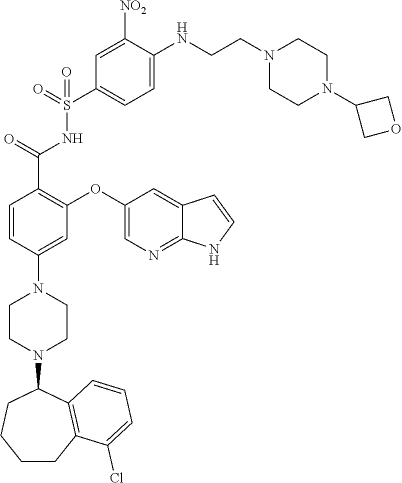 [SMILES: O=C(NS(=O)(=O)c1ccc(NCCN2CCN(C3COC3)CC2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((3-nitro-4-((2- (4-(oxetan-3-y1)piperazin-1- yl)ethyl)amino)phenyl)sulfonyl)benza- mide |
| 85 | 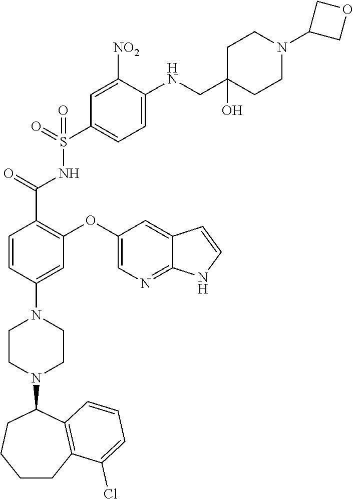 [SMILES: O=C(NS(=O)(=O)c1ccc(NCC2(O)CCN(C3COC3)CC2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((4-(((4-hydroxy- 1-(oxetan-3-yl)piperidin-4- yl)methyl)amino)-3- nitrophenyl)sulfonyl)benzamide |
| 86 | 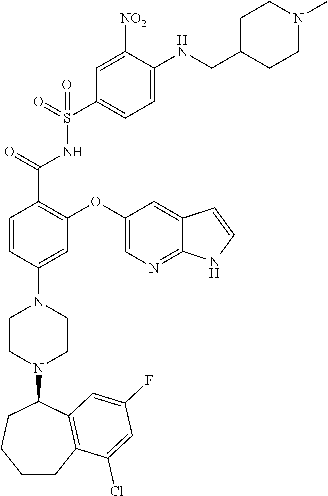 [SMILES: CN1CCC(CNc2ccc(S(=O)(=O)NC(=O)c3ccc(N4CCN([C@@H]5CCCCc6c(Cl)cc(F)cc65)CC4)cc3Oc3cnc4[nH]ccc4c3)cc2[N+](=O)[O-])CC1] | (R)-2-((1H-pyrrolo[2,3-b]pyridin-5- yl)oxy)-4-(4-(1-chloro-3-fluoro- 6,7,8,9-tetrahydro-5H- benzo[7]annulen-5-yl)piperazin-1-y1)- N-((4-(((1-methylpiperidin-4- yl)methyl)amino)-3- nitrophenyl)sulfonyl)benzamide |
| 87 | 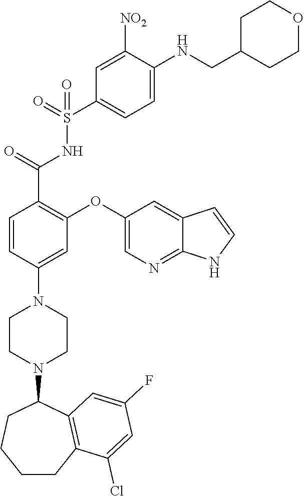 [SMILES: O=C(NS(=O)(=O)c1ccc(NCC2CCOCC2)c([N+](=O)[O-])c1)c1ccc(N2CCN([C@@H]3CCCCc4c(Cl)cc(F)cc43)CC2)cc1Oc1cnc2[nH]ccc2c1] | (R)-2-((1H-pyrrolo[2,3-b]pyridin-5- yl)oxy)-4-(4-(1-chloro-3-fluoro- 6,7,8,9-tetrahydro-5H- benzo[7]annulen-5-y1)piperazin-1-y1)- N-((3-nitro-4-(((tetrahydro-2H-pyran- 4- yl)methyl)amino)phenyl)sulfonyl)benz- amide |
| 88 | 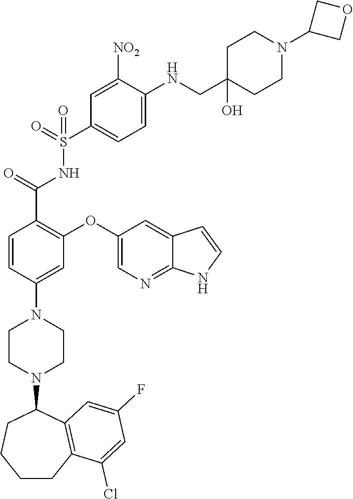 [SMILES: O=C(NS(=O)(=O)c1ccc(NCC2(O)CCN(C3COC3)CC2)c([N+](=O)[O-])c1)c1ccc(N2CCN([C@@H]3CCCCc4c(Cl)cc(F)cc43)CC2)cc1Oc1cnc2[nH]ccc2c1] | (R)-2-((1H-pyrrolo[2,3-b]pyridin-5- yl)oxy)-4-(4-(1-chloro-3-fluoro- 6,7,8,9-tetrahydro-5H- benzo[7]annulen-5-y1)piperazin-1-yl)- N-((4-(((4-hydroxy-1-(oxetan-3- yl)piperidin-4-y1)methyl)amino)-3- nitrophenyl)sulfonyl)benzamide |
| 89 | 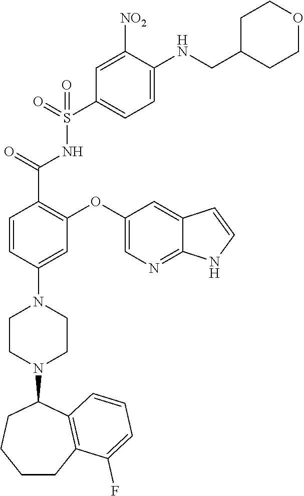 [SMILES: O=C(NS(=O)(=O)c1ccc(NCC2CCOCC2)c([N+](=O)[O-])c1)c1ccc(N2CCN([C@@H]3CCCCc4c(F)cccc43)CC2)cc1Oc1cnc2[nH]ccc2c1] | (R)-2-((1H-pyrrolo[2,3-b]pyridin-5- yl)oxy)-4-(4-(1-fluoro-6,7,8,9- tetrahydro-5H-benzo[7]annulen-5- yl)piperazin-1-y1)-N-((3-nitro-4- (((tetrahydro-2H-pyran-4- yl)methyl)amino)phenyl)sulfonyl)benz- amide |
| 90 | 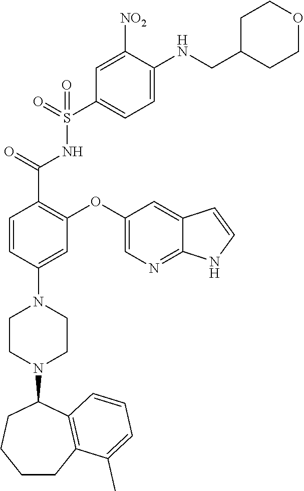 [SMILES: Cc1cccc2c1CCCC[C@H]2N1CCN(c2ccc(C(=O)NS(=O)(=O)c3ccc(NCC4CCOCC4)c([N+](=O)[O-])c3)c(Oc3cnc4[nH]ccc4c3)c2)CC1] | (R)-2-((1H-pyrrolo[2,3-b]pyridin-5- yl)oxy)-4-(4-(1-methyl-6,7,8,9- tetrahydro-5H-benzo[7]annulen-5- yl)piperazin-1-yl)-N-((3-nitro-4- (((tetrahydro-2H-pyran-4- yl)methyl)amino)phenyl)sulfonyl)benz- amide |
| 91 | 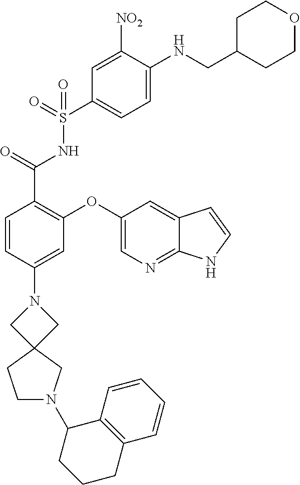 [SMILES: O=C(NS(=O)(=O)c1ccc(NCC2CCOCC2)c([N+](=O)[O-])c1)c1ccc(N2CC3(CCN(C4CCCc5ccccc54)C3)C2)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (6-(1,2,3,4-tetrahydronaphthalen-1- yl)-2,6-diazaspiro[3.4]octan-2- yl)benzamide |
| 92 | 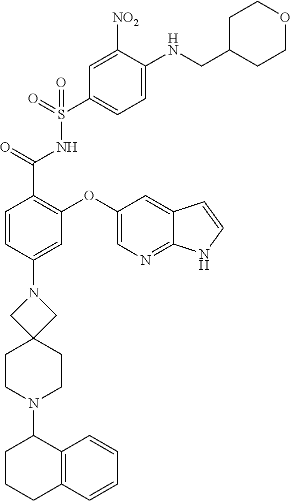 [SMILES: O=C(NS(=O)(=O)c1ccc(NCC2CCOCC2)c([N+](=O)[O-])c1)c1ccc(N2CC3(CCN(C4CCCc5ccccc54)CC3)C2)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (7-(1,2,3,4-tetrahydronaphthalen-1- yl)-2,7-diazaspiro[3.5]nonan-2- yl)benzamide |
| 93 | 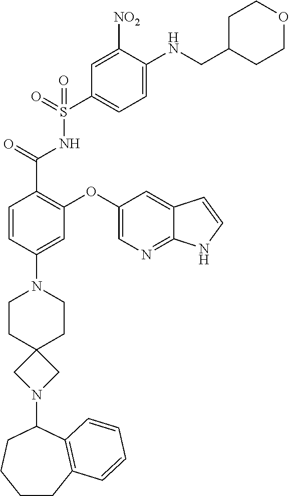 [SMILES: O=C(NS(=O)(=O)c1ccc(NCC2CCOCC2)c([N+](=O)[O-])c1)c1ccc(N2CCC3(CC2)CN(C2CCCCc4ccccc42)C3)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (2-(6,7,8,9-tetrahydro-5H- benzo[7]annulen-5-y1)-2,7- diazaspiro[3.5]nonan-7-y1)benzamide |
| 94 | 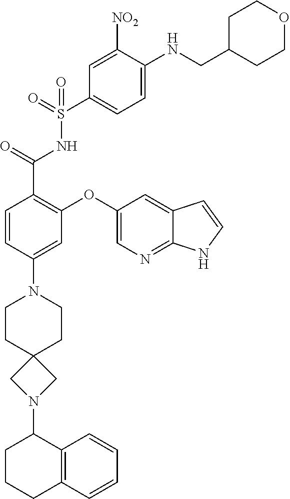 [SMILES: O=C(NS(=O)(=O)c1ccc(NCC2CCOCC2)c([N+](=O)[O-])c1)c1ccc(N2CCC3(CC2)CN(C2CCCc4ccccc42)C3)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (2-(1,2,3,4-tetrahydronaphthalen-1- yl)-2,7-diazaspiro[3.5]nonan-7- yl)benzamide |
| 95 | 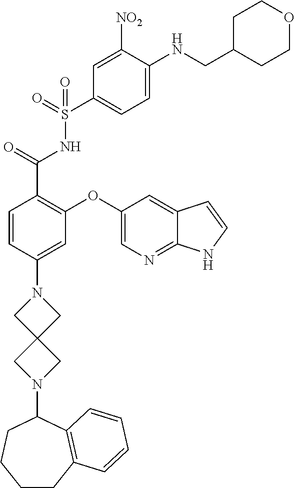 [SMILES: O=C(NS(=O)(=O)c1ccc(NCC2CCOCC2)c([N+](=O)[O-])c1)c1ccc(N2CC3(C2)CN(C2CCCCc4ccccc42)C3)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (6-(6,7,8,9-tetrahydro-5H- benzo[7]annulen-5-yl)-2,6- diazaspiro[3.3]heptan-2-yl)benzamide |
| 96 | 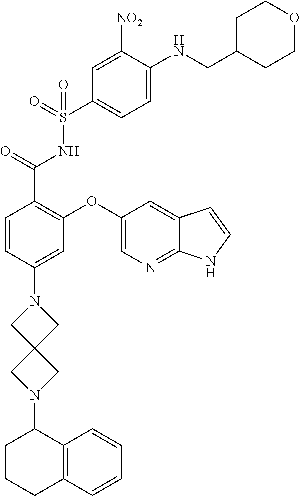 [SMILES: O=C(NS(=O)(=O)c1ccc(NCC2CCOCC2)c([N+](=O)[O-])c1)c1ccc(N2CC3(C2)CN(C2CCCc4ccccc42)C3)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (6-(1,2,3,4-tetrahydronaphthalen-1- yl)-2,6-diazaspiro[3.3]heptan-2- yl)benzamide |
| 97 | 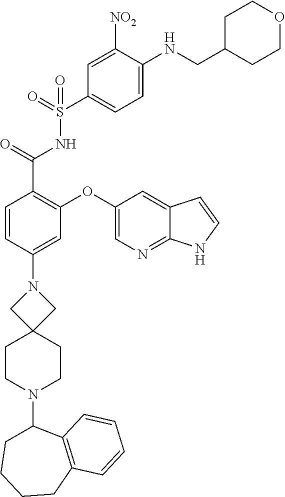 [SMILES: O=C(NS(=O)(=O)c1ccc(NCC2CCOCC2)c([N+](=O)[O-])c1)c1ccc(N2CC3(CCN(C4CCCCc5ccccc54)CC3)C2)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (7-(6,7,8,9-tetrahydro-5H- benzo[7]annulen-5-yl)-2,7- diazaspiro[3.5]nonan-2-yl)benzamide |
| 98 | 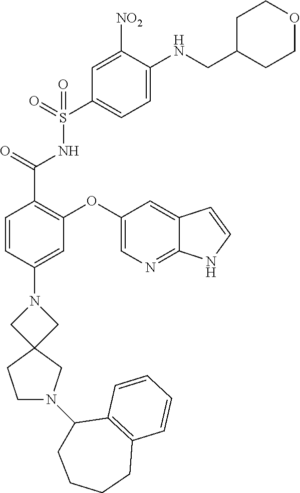 [SMILES: O=C(NS(=O)(=O)c1ccc(NCC2CCOCC2)c([N+](=O)[O-])c1)c1ccc(N2CC3(CCN(C4CCCCc5ccccc54)C3)C2)cc1Oc1cnc2[nH]ccc2c1] | 2-((1H-pyrrolo[2,3-b]pyridin-5- yl)oxy)-N-((3-nitro-4-(((tetrahydro- 2H-pyran-4- yl)methyl)amino)phenyl)sulfonyl)-4- (6-(6,7,8,9-tetrahydro-5H- benzo[7]annulen-5-yl)-2,6- diazaspiro[3.4]octan-2-yl)benzamide |
| 99 | 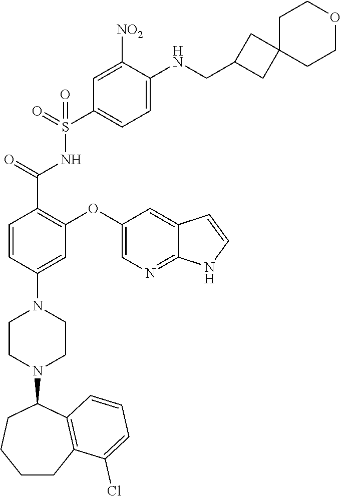 [SMILES: O=C(NS(=O)(=O)c1ccc(NCC2CC3(CCOCC3)C2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-N-((4-(((7- oxaspiro[3.5]nonan-2- yl)methyl)amino)-3- nitrophenyl)sulfony1)-4-(4-(1-chloro- 6,7,8,9-tetrahydro-5H- benzo[7]annulen-5-y1)piperazin-1- yl)benzamide |
| 100 | 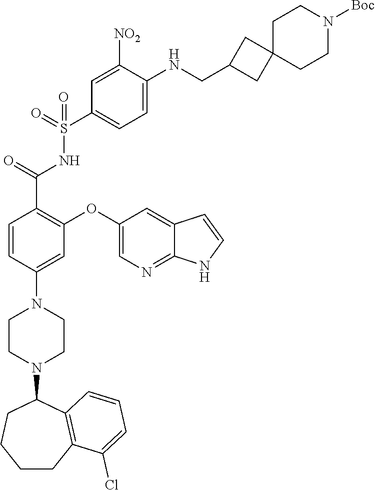 [SMILES: CC(C)(C)OC(=O)N1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | tert-butyl (R)-2-(((4-(N-(2-((1H- pyrrolo[2,3-b]pyridin-5-yl)oxy)-4-(4- (1-chloro-6,7,8,9-tetrahydro-5H- benzo[7]annulen-5-yl)piperazin-1- yl)benzoyl)sulfamoyl)-2- nitrophenyl)amino)methyl)-7- azaspiro[3.5]nonane-7-carboxylate |
| 101 | 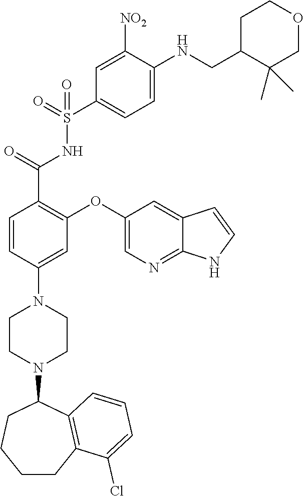 [SMILES: CC1(C)COCCC1CNc1ccc(S(=O)(=O)NC(=O)c2ccc(N3CCN([C@@H]4CCCCc5c(Cl)cccc54)CC3)cc2Oc2cnc3[nH]ccc3c2)cc1[N+](=O)[O-]] | 2-((1H-pyrrolo[2,3-b]pyridin-5- yl)oxy)-4-(4-((R)-1-chloro-6,7,8,9- tetrahydro-5H-benzo[7]annulen-5- yl)piperazin-1-yl)-N-((4-(((3,3- dimethyltetrahydro-2H-pyran-4- yl)methyl)amino)-3- nitrophenyl)sulfonyl)benzamide |
| 102 | 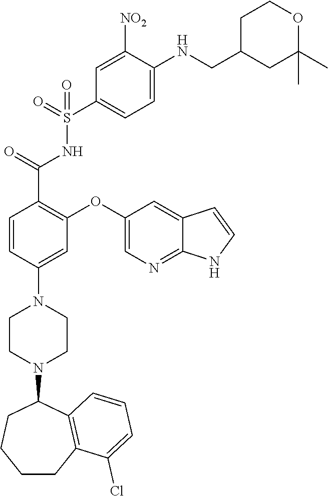 [SMILES: CC1(C)CC(CNc2ccc(S(=O)(=O)NC(=O)c3ccc(N4CCN([C@@H]5CCCCc6c(Cl)cccc65)CC4)cc3Oc3cnc4[nH]ccc4c3)cc2[N+](=O)[O-])CCO1] | 2-((1H-pyrrolo[2,3-b]pyridin-5- yl)oxy)-4-(4-((R)-1-chloro-6,7,8,9- tetrahydro-5H-benzo[7]annulen-5- yl)piperazin-1-yl)-N-((4-(((2,2- dimethyltetrahydro-2H-pyran-4- yl)methyl)amino)-3- nitrophenyl)sulfonyl)benzamide |
| 103 | 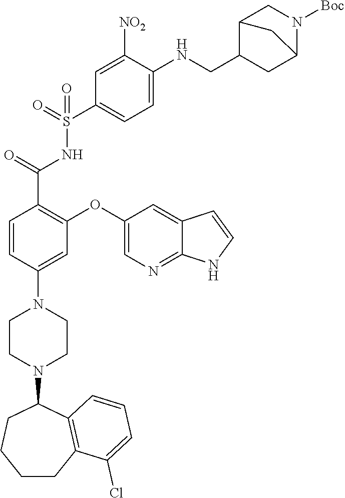 [SMILES: CC(C)(C)OC(=O)N1CC2CC1CC2CNc1ccc(S(=O)(=O)NC(=O)c2ccc(N3CCN([C@@H]4CCCCc5c(Cl)cccc54)CC3)cc2Oc2cnc3[nH]ccc3c2)cc1[N+](=O)[O-]] | tert-butyl 5-(((4-(N-(2-((1H- pyrrolo[2,3-b]pyridin-5-yl)oxy)-4-(4- ((R)-1-chloro-6,7,8,9-tetrahydro-5H- benzo[7]annulen-5-y1)piperazin-1- yl)benzoyl)sulfamoyl)-2- nitrophenyl)amino)methyl)-2- azabicyclo[2.2.1]heptane-2- carboxylate |
| 104 | 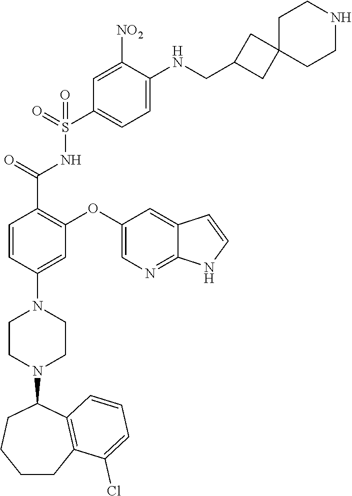 [SMILES: O=C(NS(=O)(=O)c1ccc(NCC2CC3(CCNCC3)C2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-N-((4-(((7- azaspiro[3.5]nonan-2- yl)methyl)amino)-3- nitrophenyl)sulfonyl)-4-(4-(1-chloro- 6,7,8,9-tetrahydro-5H- benzo[7]annulen-5-yl)piperazin-1- yl)benzamide |
| 105 | 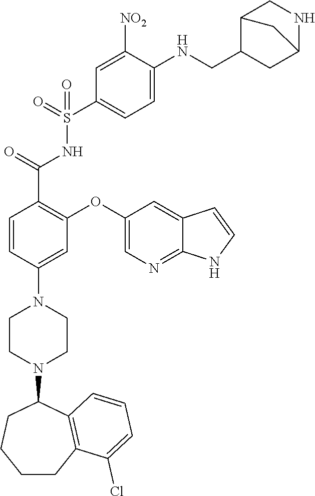 [SMILES: O=C(NS(=O)(=O)c1ccc(NCC2CC3CC2CN3)c([N+](=O)[O-])c1)c1ccc(N2CCN([C@@H]3CCCCc4c(Cl)cccc43)CC2)cc1Oc1cnc2[nH]ccc2c1] | 2-((1H-pyrrolo[2,3-b]pyridin-5- yl)oxy)-N-((4-(((2- azabicyclo[2.2.1 ]heptan-5- yl)methyl)amino)-3- nitrophenyl)sulfonyl)-4-(4-((R)-1- chloro-6,7,8,9-tetrahydro-5H- benzo[7]annulen-5-y1)piperazin-1- yl)benzamide |
| 106 | 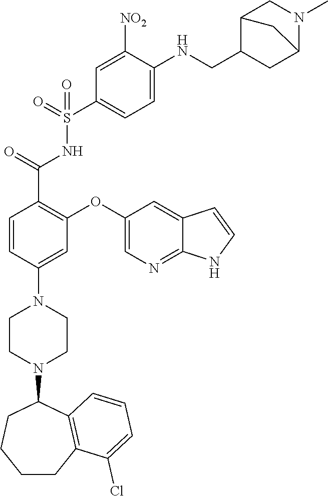 [SMILES: CN1CC2CC1CC2CNc1ccc(S(=O)(=O)NC(=O)c2ccc(N3CCN([C@@H]4CCCCc5c(Cl)cccc54)CC3)cc2Oc2cnc3[nH]ccc3c2)cc1[N+](=O)[O-]] | 2-((1H-pyrrolo[2,3-b]pyridin-5- yl)oxy)-4-(4-((R)-1-chloro-6,7,8,9- tetrahydro-5H-benzo[7]annulen-5- yl)piperazin-1-y1)-N-((4-(((2-methyl- 2-azabicyclo[2.2.1]heptan-5- yl)methyl)amino)-3- nitrophenyl)sulfonyl)benzamide |
| 107 | 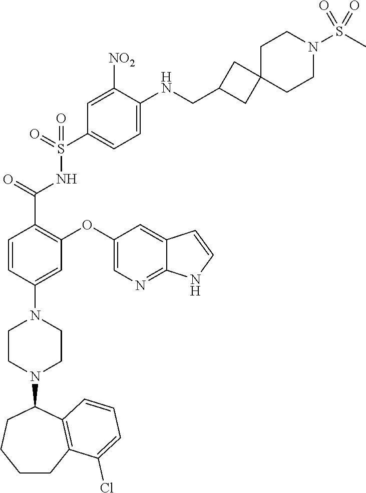 [SMILES: CS(=O)(=O)N1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-y1)-N-((4-(((7- (methylsulfonyl)-7- azaspiro[3.5]nonan-2- yl)methyl)amino)-3- nitrophenyl)sulfonyl)benzamide |
| 108 | 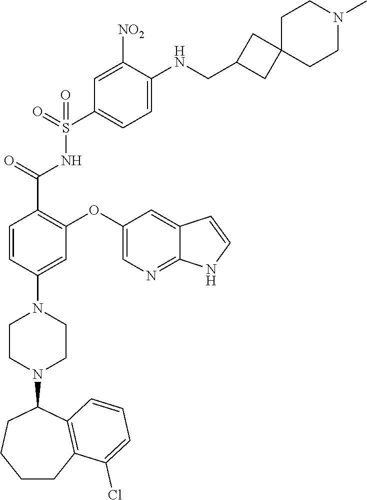 [SMILES: CN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((4-(((7-methyl- 7-azaspiro[3.5]nonan-2- yl)methyl)amino)-3- nitrophenyl)sulfonyl)benzamide |
| 109 | 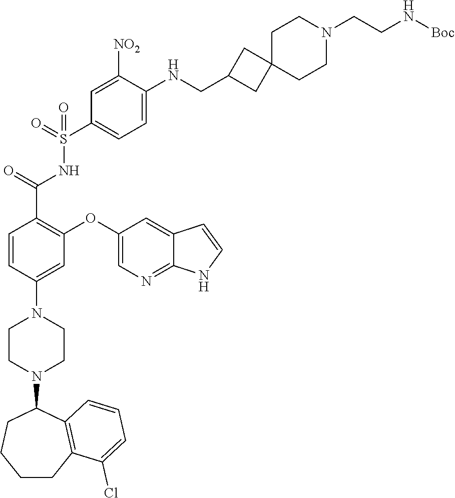 [SMILES: CC(C)(C)OC(=O)NCCN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | tert-butyl (R)-(2-(2-(((4-(N-(2-((1H- pyrrolo[2,3-b]pyridin-5-yl)oxy)-4-(4- (1-chloro-6,7,8,9-tetrahydro-5H- benzo[7]annulen-5-yl)piperazin-1- yl)benzoyl)sulfamoy1)-2- nitrophenyl)amino)methyl)-7- azaspiro[3.5]nonan-7- yl)ethyl)carbamate |
| 110 | 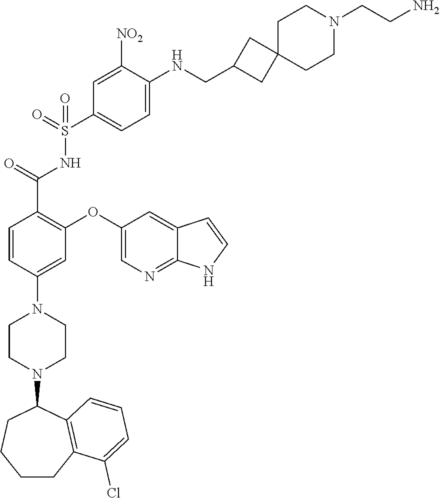 [SMILES: NCCN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | (R)-2-((1H-pyrrolo[2,3-b]pyridin-5- yl)oxy)-N-((4-(((7-(2-aminoethyl)-7- azaspiro[3.5]nonan-2- yl)methyl)amino)-3- nitrophenyl)sulfony1)-4-(4-(1-chloro- 6,7,8,9-tetrahydro-5H- benzo[7]annulen-5-yl)piperazin-1- yl)benzamide |
| 111 | 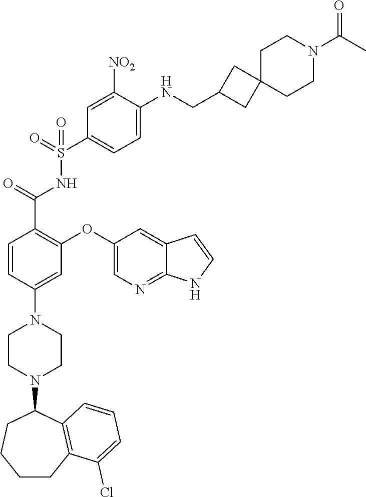 [SMILES: CC(=O)N1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | (R)-2-((1H-pyrrolo[2,3-b]pyridin-5- yl)oxy)-N-((4-(((7-acetyl-7- azaspiro[3.5]nonan-2- yl)methyl)amino)-3- nitrophenyl)sulfonyl)-4-(4-(1-chloro- 6,7,8,9-tetrahydro-5H- benzo[7]annulen-5-yl)piperazin-1- yl)benzamide |
| 112 | 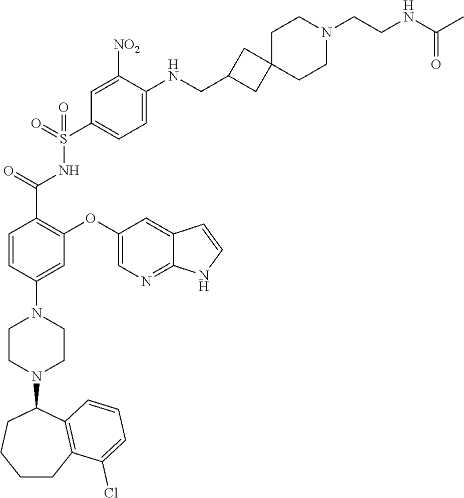 [SMILES: CC(=O)NCCN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | (R)-2-((1H-pyrrolo[2,3-b]pyridin-5- yl)oxy)-N-((4-(((7-(2-acetamidoethyl)- 7-azaspiro[3.5]nonan-2- yl)methyl)amino)-3- nitrophenyl)sulfony1)-4-(4-(1-chloro- 6,7,8,9-tetrahydro-5H- benzo[7]annulen-5-yl)piperazin-1- yl)benzamide |
| 113 | 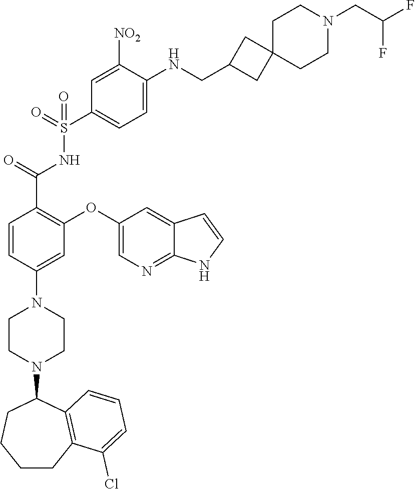 [SMILES: O=C(NS(=O)(=O)c1ccc(NCC2CC3(CCN(CC(F)F)CC3)C2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-y1)-N-((4-(((7-(2,2- difluoroethyl)-7-azaspiro[3.5]nonan-2- yl)methyl)amino)-3- nitrophenyl)sulfonyl)benzamide |
| 114 | 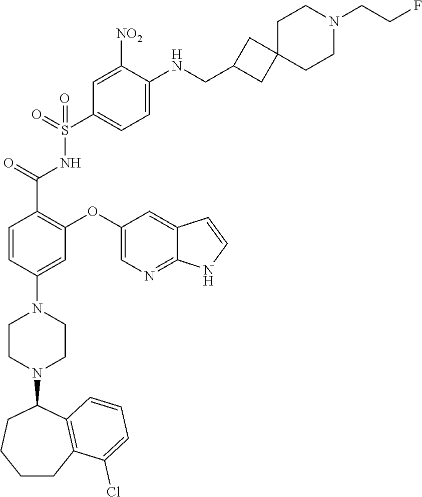 [SMILES: O=C(NS(=O)(=O)c1ccc(NCC2CC3(CCN(CCF)CC3)C2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-y1)-N-((4-(((7-(2- fluoroethyl)-7-azaspiro[3.5]nonan-2- yl)methyl)amino)-3- nitrophenyl)sulfonyl)benzamide |
| 115 | 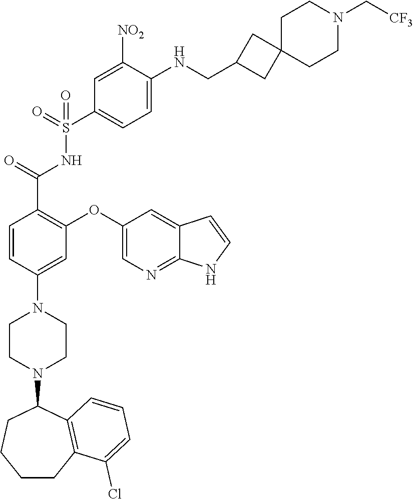 [SMILES: O=C(NS(=O)(=O)c1ccc(NCC2CC3(CCN(CC(F)(F)F)CC3)C2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((3-nitro-4-(((7- (2,2,2-trifluoroethyl)-7- azaspiro[3.5]nonan-2- yl)methyl)amino)phenyl)sulfonyl)benz- amide |
| 116 | 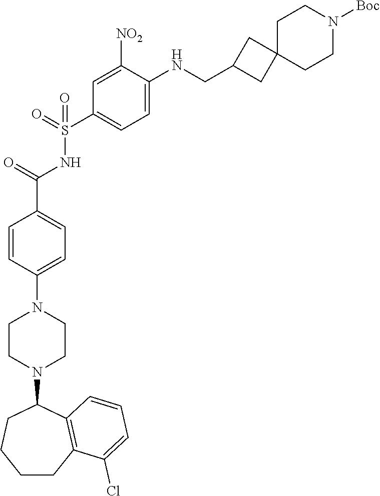 [SMILES: CC(C)(C)OC(=O)N1CCC2(CC1)CC(CNc1ccc(S(=O)(=O)NC(=O)c3ccc(N4CCN([C@@H]5CCCCc6c(Cl)cccc65)CC4)cc3)cc1[N+](=O)[O-])C2] | tert-butyl (R)-2-(((4-(N-(4-(4-(1- chloro-6,7,8,9-tetrahydro-5H- benzo[7]annulen-5-yl)piperazin-1- yl)benzoyl)sulfamoyl)-2- nitrophenyl)amino)methyl)-7- azaspiro[3.5]nonane-7-carboxylate |
| 117 | 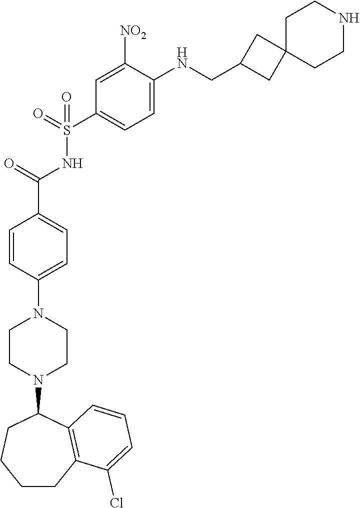 [SMILES: O=C(NS(=O)(=O)c1ccc(NCC2CC3(CCNCC3)C2)c([N+](=O)[O-])c1)c1ccc(N2CCN([C@@H]3CCCCc4c(Cl)cccc43)CC2)cc1] | (R)-N-((4-(((7-azaspiro[3.5]nonan-2- yl)methyl)amino)-3- nitrophenyl)sulfonyl)-4-(4-(1-chloro- 6,7,8,9-tetrahydro-5H- benzo[7]annulen-5-y1)piperazin-1- yl)benzamide |
| 118 | 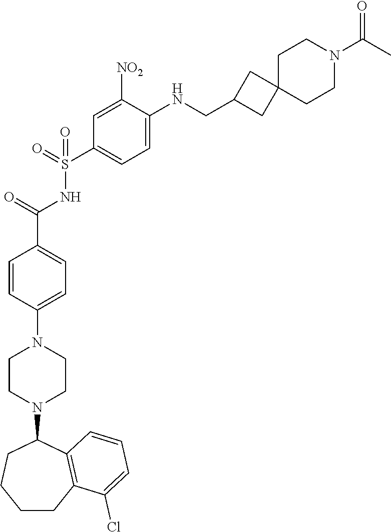 [SMILES: CC(=O)N1CCC2(CC1)CC(CNc1ccc(S(=O)(=O)NC(=O)c3ccc(N4CCN([C@@H]5CCCCc6c(Cl)cccc65)CC4)cc3)cc1[N+](=O)[O-])C2] | (R)-N-((4-(((7-acetyl-7- azaspiro[3.5]nonan-2- yl)methyl)amino)-3- nitrophenyl)sulfony1)-4-(4-(1-chloro- 6,7,8,9-tetrahydro-5H- benzo[7]annulen-5-y1)piperazin-1- yl)benzamide |
| 119 | 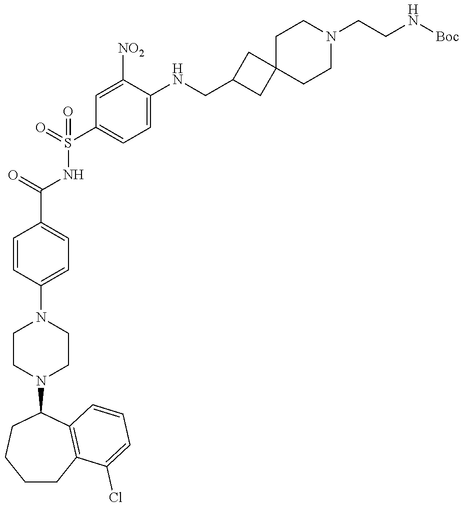 [SMILES: CC(C)(C)OC(=O)NCCN1CCC2(CC1)CC(CNc1ccc(S(=O)(=O)NC(=O)c3ccc(N4CCN([C@@H]5CCCCc6c(Cl)cccc65)CC4)cc3)cc1[N+](=O)[O-])C2] | tert-butyl (R)-(2-(2-(((4-(N-(4-(4-(1- chloro-6,7,8,9-tetrahydro-5H- benzo[7]annulen-5-yl)piperazin-1- yl)benzoyl)sulfamoyl)-2- nitrophenyl)amino)methyl)-7- azaspiro[3.5]nonan-7- yl)ethyl)carbamate |
| 120 | 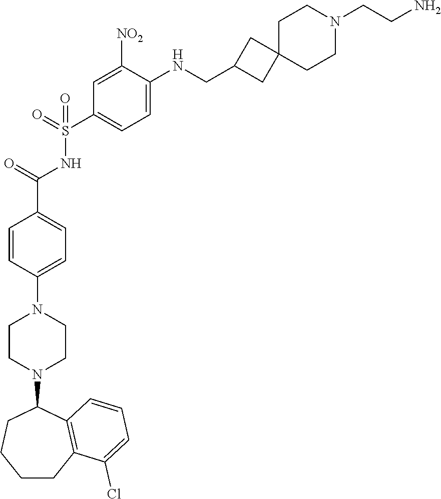 [SMILES: NCCN1CCC2(CC1)CC(CNc1ccc(S(=O)(=O)NC(=O)c3ccc(N4CCN([C@@H]5CCCCc6c(Cl)cccc65)CC4)cc3)cc1[N+](=O)[O-])C2] | (R)-N-((4-(((7-(2-aminoethyl)-7- azaspiro[3.5]nonan-2- yl)methyl)amino)-3- nitrophenyl)sulfonyl)-4-(4-(1-chloro- 6,7,8,9-tetrahydro-5H- benzo[7]annulen-5-yl)piperazin-1- yl)benzamide |
| 121 | 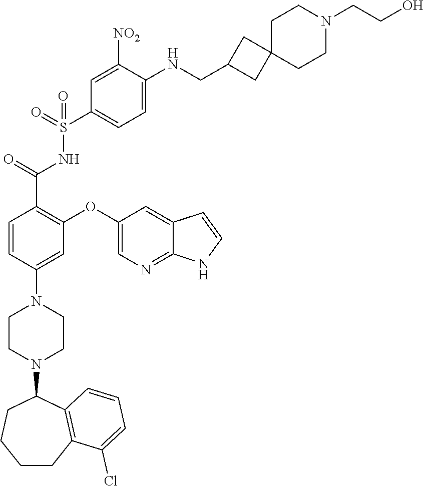 [SMILES: O=C(NS(=O)(=O)c1ccc(NCC2CC3(CCN(CCO)CC3)C2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-y1)-N-((4-(((7-(2- hydroxyethyl)-7-azaspiro[3.5]nonan- 2-yl)methyl)amino)-3- nitrophenyl)sulfonyl)benzamide |
| 122 | 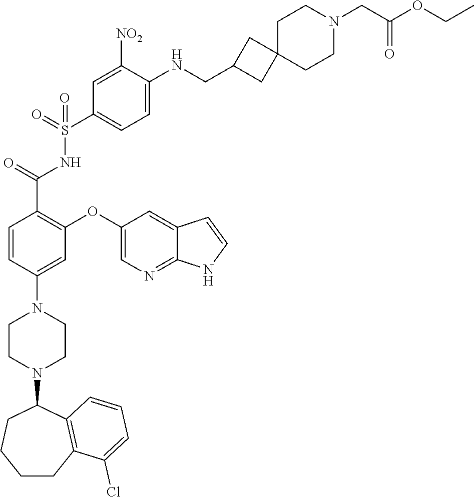 [SMILES: CCOC(=O)CN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | ethyl (R)-2-(2-(((4-(N-(2-((1H- pyrrolo[2,3-b]pyridin-5-yl)oxy)-4-(4- (1-chloro-6,7,8,9-tetrahydro-5H- benzo[7]annulen-5-y1)piperazin-1- yl)benzoyl)sulfamoyl)-2- nitrophenyl)amino)methyl)-7- azaspiro[3.5]nonan-7-yl)acetate |
| 123 | 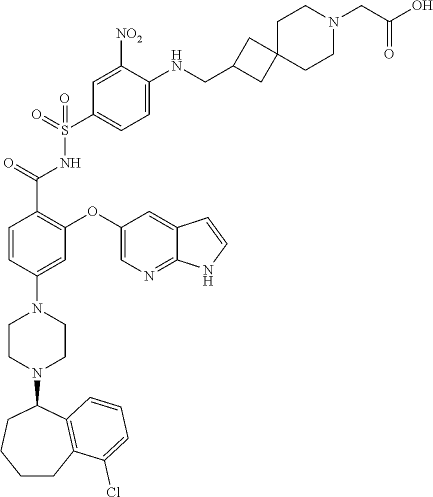 [SMILES: O=C(O)CN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | (R)-2-(2-(((4-(N-(2-((1H-pyrrolo[2,3- b]pyridin-5-yl)oxy)-4-(4-(1-chloro- 6,7,8,9-tetrahydro-5H- benzo[7]annulen-5-yl)piperazin-1- yl)benzoyl)sulfamoy1)-2- nitrophenyl)amino)methyl)-7- azaspiro[3.5]nonan-7-yl)acetic acid |
| 124 | 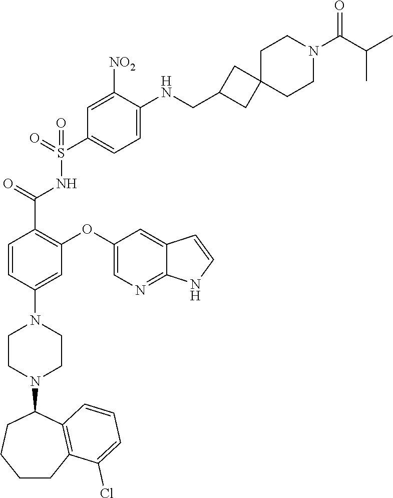 [SMILES: CC(C)C(=O)N1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-y1)-N-((4-(((7- isobutyry1-7-azaspiro[3.5]nonan-2- yl)methyl)amino)-3- nitrophenyl)sulfonyl)benzamide |
| 125 | 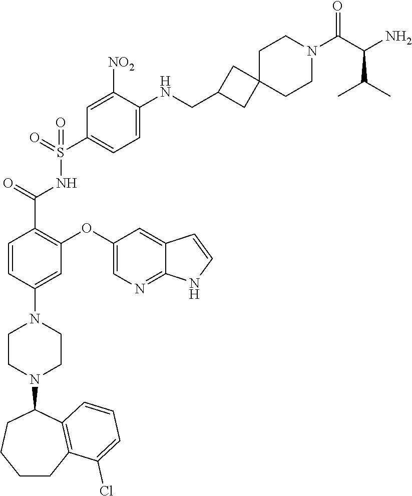 [SMILES: CC(C)[C@H](N)C(=O)N1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | 2-((1H-pyrrolo[2,3-b]pyridin-5- yl)oxy)-N-((4-(((7-(L-valyl)-7- azaspiro[3.5]nonan-2- yl)methyl)amino)-3- nitrophenyl)sulfony1)-4-(4-((R)-1- chloro-6,7,8,9-tetrahydro-5H- benzo[7]annulen-5-yl)piperazin-1- yl)benzamide |
| 126 | 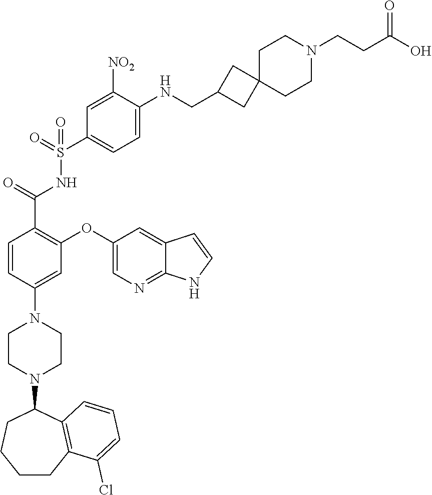 [SMILES: O=C(O)CCN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | (R)-3-(2-(((4-(N-(2-((1H-pyrrolo[2,3- b]pyridin-5-yl)oxy)-4-(4-(1-chloro- 6,7,8,9-tetrahydro-5H- benzo[7]annulen-5-yl)piperazin-1- yl)benzoyl)sulfamoyl)-2- nitrophenyl)amino)methyl)-7- azaspiro[3.5]nonan-7-yl)propanoic acid |
| 127 | 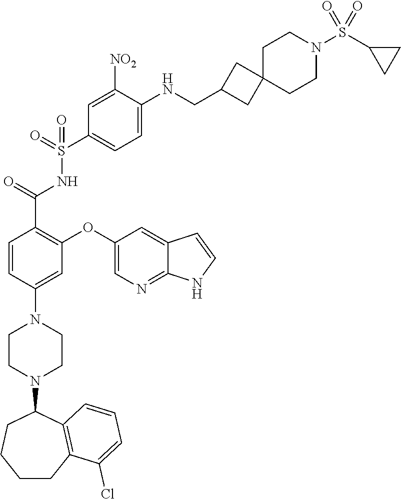 [SMILES: O=C(NS(=O)(=O)c1ccc(NCC2CC3(CCN(S(=O)(=O)C4CC4)CC3)C2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-y1)-N-((4-(((7- (cyclopropylsulfonyl)-7- azaspiro[3.5]nonan-2- yl)methyl)amino)-3- nitrophenyl)sulfonyl)benzamide |
| 128 | 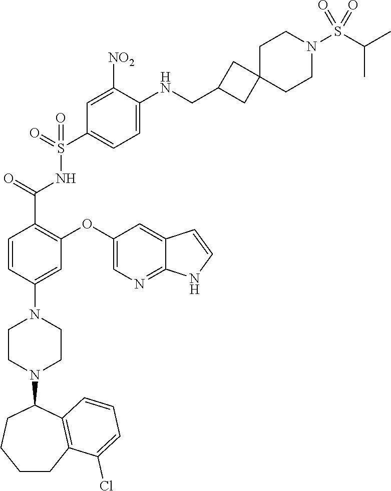 [SMILES: CC(C)S(=O)(=O)N1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((4-(((7- (isopropylsulfonyl)-7- azaspiro[3.5]nonan-2- yl)methyl)amino)-3- nitrophenyl)sulfonyl)benzamide |
| 129 | 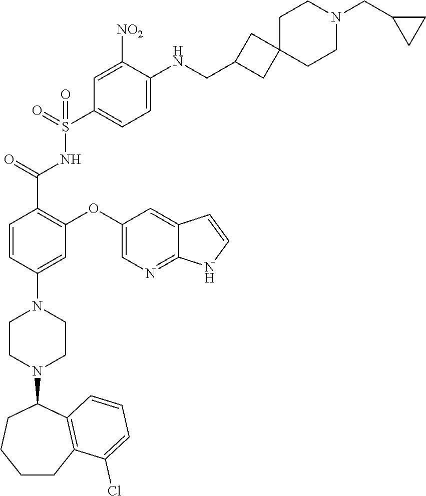 [SMILES: O=C(NS(=O)(=O)c1ccc(NCC2CC3(CCN(CC4CC4)CC3)C2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((4-(((7- (cyclopropylmethyl)-7- azaspiro[3.5]nonan-2- yl)methyl)amino)-3- nitrophenyl)sulfonyl)benzamide |
| 130 | 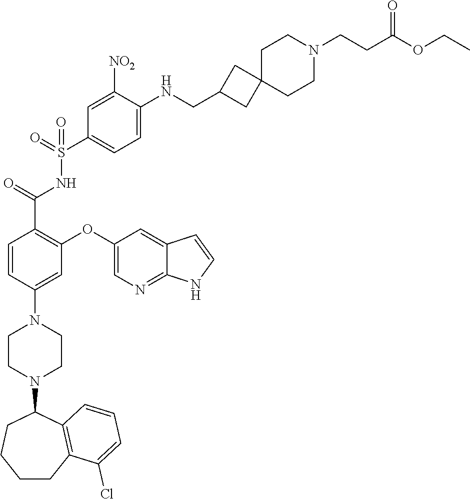 [SMILES: CCOC(=O)CCN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | ethyl (R)-3-(2-(((4-(N-(2-((1H- pyrrolo[2,3-b]pyridin-5-yl)oxy)-4-(4- (1-chloro-6,7,8,9-tetrahydro-5H- benzo[7]annulen-5-yl)piperazin-1- yl)benzoyl)sulfamoyl)-2- nitrophenyl)amino)methyl)-7- azaspiro[3.5]nonan-7-yl)propanoate |
| 131 | 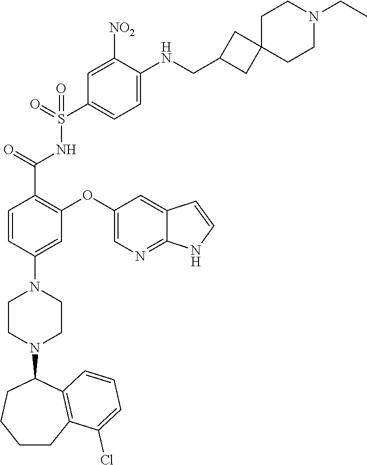 [SMILES: CCN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((4-(((7-ethyl-7- azaspiro[3.5]nonan-2- yl)methyl)amino)-3- nitrophenyl)sulfonyl)benzamide |
| 132 | 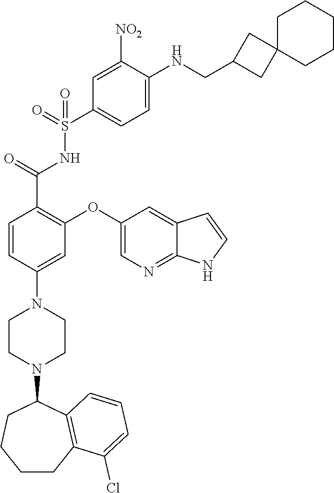 [SMILES: O=C(NS(=O)(=O)c1ccc(NCC2CC3(CCCCC3)C2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((3-nitro-4- ((spiro[3.5]nonan-2- ylmethyl)amino)phenyl)sulfonyl)benz- amide |
| 133 | 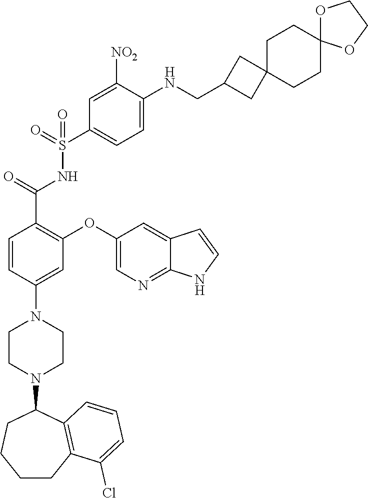 [SMILES: O=C(NS(=O)(=O)c1ccc(NCC2CC3(CCC4(CC3)OCCO4)C2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-N-((4-(((8,11- dioxadispiro[3.2.47.24]tridecan-2- yl)methyl)amino)-3- nitrophenyl)sulfonyl)-4-(4-(1-chloro- 6,7,8,9-tetrahydro-5H- benzo[7]annulen-5-y1)piperazin-1- yl)benzamide |
| 134 | 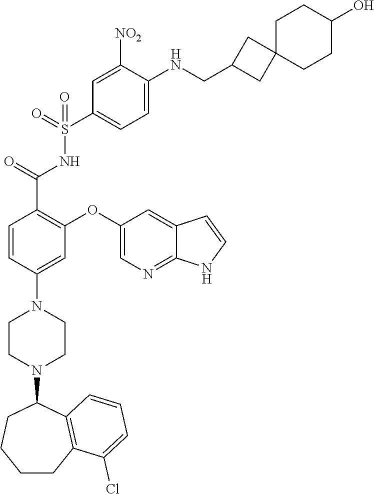 [SMILES: O=C(NS(=O)(=O)c1ccc(NCC2CC3(CCC(O)CC3)C2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-y1)-N-((4-(((7- hydroxyspiro[3.5]nonan-2- yl)methyl)amino)-3- nitrophenyl)sulfonyl)benzamide |
| 135 | 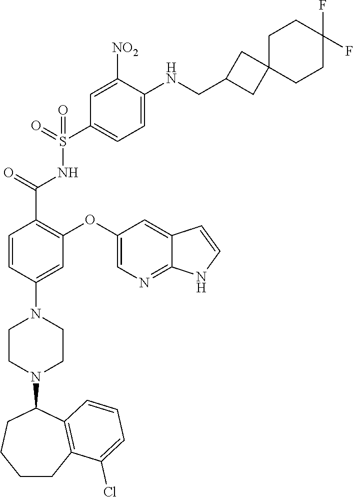 [SMILES: O=C(NS(=O)(=O)c1ccc(NCC2CC3(CCC(F)(F)CC3)C2)c([N+](=O)[O-])c1)c1ccc(N2CCN([C@@H]3CCCCc4c(Cl)cccc43)CC2)cc1Oc1cnc2[nH]ccc2c1] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-y1)-N-((4-(((7,7- difluorospiro[3.5]nonan-2- yl)methyl)amino)-3- nitrophenyl)sulfonyl)benzamide |
| 136 | 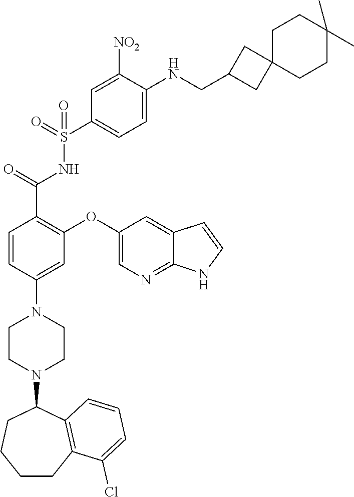 [SMILES: CC1(C)CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2] | (R)-2-((1H-pyrrolo[2,3-b]pyridin-5- yl)oxy)-4-(4-(1-chloro-6,7,8,9- tetrahydro-5H-benzo[7]annulen-5- yl)piperazin-1-yl)-N-((4-(((7,7- dimethylspiro[3.5]nonan-2- yl)methyl)amino)-3- nitrophenyl)sulfonyl)benzamide |

Compounds provided herein are described with reference to both generic formulae and specific compounds. In addition, the compounds of the present disclosure may exist in a number of different forms or derivatives, including but not limited to, stereoisomers, racemic mixtures, regioisomers, tautomers, salts, prodrugs, soft drugs, active metabolic derivatives (active metabolites), solvated forms, different crystal forms or polymorphs, all within the scope of the present disclosure.

The compounds of present disclosure can comprise one or more asymmetric centers, and thus can exist in various stereoisomeric forms, e.g., enantiomers and/or diastereomers. Thus, the compounds of present disclosure and compositions thereof may be in the form of an individual enantiomer, diastereomer or geometric isomer, or may be in the form of a mixture of stereoisomers. In certain embodiments, the compounds of the present disclosure are enantiopure compounds. In certain embodiments, mixtures of enantiomers or diastereomers are provided.

The term “enantiomer” refers to two stereoisomers of a compound which are non-superimposable mirror images of one another. The term “diastereomer” refers to a pair of optical isomers which are not mirror images of one another. Diastereomers have different physical properties, e.g. melting points, boiling points, spectral properties, and reactivities.

Furthermore, certain compounds, as described herein may have one or more double bonds that can exist as either the Z or E isomer, unless otherwise indicated. The present disclosure additionally encompasses the compounds as individual isomers substantially free of other isomers and alternatively, as mixtures of various isomers, e.g., racemic mixtures of enantiomers. In addition to the above-mentioned compounds per se, this disclosure also encompasses compositions comprising one or more compounds.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, a stereoisomer may, in some embodiments, be provided substantially free of one or more corresponding stereoisomers, and may also be referred to as “stereochemically enriched”.

Where a particular enantiomer is preferred, it may, in some embodiments be provided substantially free of the opposite enantiomer, and may also be referred to as “optically enriched”. “Optically enriched”, as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high performance liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, IN 1972).

The compounds of the present disclosure may also exist in different tautomeric forms, and all such forms are embraced within the scope of the present disclosure. The term “tautomer” or “tautomeric form” refers to structural isomers of different energies which are interconvertible via a low energy barrier. The presence and concentrations of the isomeric forms will depend on the environment the compound is found in and may be different depending upon, for example, whether the compound is a solid or is in an organic or aqueous solution. By way of examples, proton tautomers (also known as prototropic tautomers) include interconversions via migration of a proton, such as keto-enol, amide-imidic acid, lactam-lactim, imine-enamine isomerizations and annular forms where a proton can occupy two or more positions of a heterocyclic system. Valence tautomers include interconversions by reorganization of some of the bonding electrons. Tautomers can be in equilibrium or sterically locked into one form by appropriate substitution. Compounds of the present disclosure identified by name or structure as one particular tautomeric form are intended to include other tautomeric forms unless otherwise specified.

As used herein, the term “prodrug” refers to compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolism, the ester group is cleaved to yield the active drug. Also, some prodrugs are activated enzymatically to yield the active compound, or a compound which, upon further chemical reaction, yields the active compound. Prodrugs may proceed from prodrug form to active form in a single step or may have one or more intermediate forms which may themselves have activity or may be inactive. Preparation and use of prodrugs are discussed in T. Higuchi and V. Stella, “Pro-drugs as Novel Delivery Systems”, Vol. 14 of the A.C.S. Symposium Series, in Bioreversible Carriers in Drug Design, ed. Edward B. Roche, American Pharmaceutical Association and Pergamon Press, 1987; in Prodrugs: Challenges and Rewards, ed. V. Stella, R. Borchardt, M. Hageman, R. Oliyai, H. Maag, J. Tilley, Springer-Verlag New York, 2007, all of which are hereby incorporated by reference in their entireties.

As used herein, the term “soft drug” refers to compounds that exert a pharmacological effect but break down to inactive metabolites degradants so that the activity is of limited time. See, for example, “Soft drugs: Principles and methods for the design of safe drugs”, Nicholas Bodor, Medicinal Research Reviews, Vol. 4, No. 4, 449-469, 1984, which is hereby incorporated by reference in its entirety.

As used herein, the term “metabolite”, e.g., active metabolite overlaps with prodrug as described above. Thus, such metabolites are pharmacologically active compounds or compounds that further metabolize to pharmacologically active compounds that are derivatives resulting from metabolic process in the body of a subject. For example, such metabolites may result from oxidation, reduction, hydrolysis, amidation, deamidation, esterification, deesterification, enzymatic cleavage, and the like, of the administered compound or salt or prodrug. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques known in the art. See, e.g., Bertolini et al., 1997, J Med Chem 40:2011-2016; Shan et al., J Pharm Sci 86:756-757; Bagshawe, 1995, Drug Dev Res 34:220-230.

As used herein, the term “active intermediate” refers to an intermediate compound in the synthetic process, which exhibits the same or essentially the same biological activity as the final synthesized compound.

Compounds of the present disclosure can be formulated as or be in the form of pharmaceutically acceptable salts. Unless specified to the contrary, a compound provided herein includes pharmaceutically acceptable salts of such compound.

As used herein, the term “pharmaceutically acceptable” indicates that the substance or composition is compatible chemically and/or toxicologically, with the other ingredients comprising a formulation, and/or the subjects being treated therewith.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, PA, Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the compounds of present disclosure can exist in unsolvated forms, solvated forms (e.g., hydrated forms), and solid forms (e.g., crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, then the solvate formed is a hydrate; and if the solvent is alcohol, then the solvate formed is an alcoholate. Hydrates are formed by the combination of one or more molecules of water with one molecule of the substance in which the water retains its molecular state as H₂O. Examples of solvents that form solvates include, but are not limited to, water, isopropanol, ethanol, methanol, DMSO, ethyl acetate, acetic acid, and ethanolamine.

As used herein, the terms “crystal form”, “crystalline form”, “polymorphic forms” and “polymorphs” can be used interchangeably, and mean crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The present disclosure is also intended to include all isotopes of atoms in the compounds. Isotopes of an atom include atoms having the same atomic number but different mass numbers. For example, unless otherwise specified, hydrogen, carbon, nitrogen, oxygen, phosphorous, sulfur, fluorine, chlorine, bromide or iodine in the compounds of present disclosure are meant to also include their isotopes, such as but not limited to ¹H, ²H, ³H, ¹¹C, ¹²C, ¹³C, ¹⁴C, ¹⁴N, ⁵N, ¹⁶O, ¹⁷O, ¹⁸O, ³¹P, ³²P, ³²S, ³³S, ³⁴S, ³⁶S, ¹⁷F, ¹⁸F, ¹⁹F, ³⁵Cl, ³⁷Cl, ⁷⁹Br, ⁸¹Br, ¹²⁴I, ¹²⁷I and ¹³¹I. In some embodiments, hydrogen includes protium, deuterium and tritium. In some embodiments, carbon includes ¹²C and ¹³C.

### Synthesis of Compounds

Synthesis of the compounds provided herein, including pharmaceutically acceptable salts thereof, are illustrated in the synthetic schemes in the examples. The compounds provided herein can be prepared using any known organic synthesis techniques and can be synthesized according to any of numerous possible synthetic routes, and thus these schemes are illustrative only and are not meant to limit other possible methods that can be used to prepare the compounds provided herein. Additionally, the steps in the Schemes are for better illustration and can be changed as appropriate. The embodiments of the compounds in examples were synthesized for the purposes of research and potentially submission to regulatory agencies.

The reactions for preparing compounds of the present disclosure can be carried out in suitable solvents, which can be readily selected by one skilled in the art of organic synthesis. Suitable solvents can be substantially non-reactive with the starting materials (reactants), the intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

The structures of the compounds in the examples are characterized by nuclear magnetic resonance (NMR) or/and liquid chromatography-mass spectrometry (LC-MS). NMR chemical shift (δ) is given in the unit of 10⁻⁶ (ppm). ¹H-NMR spectra is recorded in CDCl₃, CD₃OD or DMSO-d₆ solutions (reported in ppm) on a Bruker instrument (400 MHz or 500 MHz), using tetramethylsilane (TMS) as the reference standard (0.0 ppm).

MS measurement was carried out using an Agilent G6100 series Mass Spectrometer using electrospray, chemical and electron impact ionization methods from a range of instruments.

TLC measurement was carried out using Shanghai Yu Cheng plates. The silica gel plates used for TLC are 0.20 mm-0.25 mm. The silica gel plates used for separating and purifying products by TLC are 1 mm.

Column chromatography was done on a Biotage system (Manufacturer: Biotage Sweden AB) having a silica gel column or on a silica cartridge.

The known starting materials of the present disclosure can be synthesized by using or according to the known methods in the art, or can be purchased from commercial suppliers such as Adamas-beta, Bidepharm or Accela ChemBio Co., Ltd, and were used without further purification unless otherwise indicated.

Unless otherwise specified, the reactions of the present disclosure were typically done under a positive pressure of nitrogen or argon or with a drying tube in anhydrous solvents, and the reaction flasks were typically fitted with rubber septa for the introduction of substrates and reagents via syringe. Glassware was oven dried and/or heat dried.

### Uses of Compounds

In one aspect, the present disclosure provides compounds of Formula I, Formula II, Formula III, Formula IV, Formula IV(a), Formula IV(b), Formula IV(c), Formula IV(d), Formula IV(e), or tautomers, stereoisomers, pharmaceutically acceptable salts thereof, which show BCL-2 or BCL-2/BCL-XL dual inhibitory activity.

As used herein, the term “BCL-2/BCL-XL” refers to both BCL-2 and BCL-XL.

As used herein, the term “BCL-2 inhibitory activity” refers to a decrease in the level or activity of BCL-2 as a direct or indirect response to the presence of a compound of Formula I, Formula II, Formula III, Formula IV, Formula IV(a), Formula IV(b), Formula IV(c), Formula IV(d), Formula IV(e), or a tautomer, stereoisomer, pharmaceutically acceptable salt thereof, relative to the level or activity of BCL-2 in the absence of a compound of Formula I, Formula II, Formula III, Formula IV, Formula IV(a), Formula IV(b), Formula IV(c), Formula IV(d), Formula IV(e), or a tautomer, stereoisomer, pharmaceutically acceptable salt thereof. Such a decrease in the level or activity may be due to the direct interaction of the compound of Formula I, Formula II, Formula III, Formula IV, Formula IV(a), Formula IV(b), Formula IV(c), Formula IV(d), Formula IV(e), or a tautomer, stereoisomer, pharmaceutically acceptable salt thereof with BCL-2, or due to the interaction of the compound of Formula I, Formula II, Formula III, Formula IV, Formula IV(a), Formula IV(b), Formula IV(c), Formula IV(d), Formula IV(e), or a tautomer, stereoisomer, pharmaceutically acceptable salt thereof with one or more other factors that in turn affect BCL-2 level or activity. For example, the compound of Formula I, Formula II, Formula III, Formula IV, Formula IV(a), Formula IV(b), Formula IV(c), Formula IV(d), Formula IV(e), or a tautomer, stereoisomer, pharmaceutically acceptable salt thereof may decrease BCL-2 by directly binding to the BCL-2 protein, by causing (directly or indirectly) another factor to decrease BCL-2 activity, or by (directly or indirectly) decreasing the amount of BCL-2 protein present in the cell or organism.

As used herein, the term “BCL-2/BCL-XL dual inhibitory activity” refers to a decrease in the level or activity of BCL-2 and BCL-XL as a direct or indirect response to the presence of a compound of Formula I, Formula II, Formula III, Formula IV, Formula IV(a), Formula IV(b), Formula IV(c), Formula IV(d), Formula IV(e), or a tautomer, stereoisomer, pharmaceutically acceptable salt thereof, relative to the level or activity of BCL-2 and BCL-XL in the absence of compound of Formula I, Formula II, Formula III, Formula IV, Formula IV(a), Formula IV(b), Formula IV(c), Formula IV(d), Formula IV(e), or a tautomer, stereoisomer, pharmaceutically acceptable salt thereof. Such a decrease in level or activity may be due to the direct interaction of the compound of Formula I, Formula II, Formula III, Formula IV, Formula IV(a), Formula IV(b), Formula IV(c), Formula IV(d), Formula IV(e), or a tautomer, stereoisomer, pharmaceutically acceptable salt thereof with BCL-2 and BCL-XL, or due to the interaction of the compound of Formula I, Formula II, Formula III, Formula IV, Formula IV(a), Formula IV(b), Formula IV(c), Formula IV(d), Formula IV(e), or a tautomer, stereoisomer, pharmaceutically acceptable salt thereof with one or more other factors that in turn affect BCL-2 and BCL-XL level or activity. For example, the compound of Formula I, Formula II, Formula III, Formula IV, Formula IV(a), Formula IV(b), Formula IV(c), Formula IV(d), Formula IV(e), or a tautomer, stereoisomer, pharmaceutically acceptable salt thereof may decrease BCL-2 and BCL-XL by directly binding to the BCL-2 and BCL-XL proteins, by causing (directly or indirectly) another factor to decrease BCL-2 and BCL-XL activities, or by (directly or indirectly) decreasing the amounts of BCL-2 and BCL-XL proteins present in the cell or organism.

In some embodiments, the compounds of the present disclosure are selective inhibitors of BCL-2.

As used herein, the term “BCL-2 selective inhibitor” or “selectively inhibits BCL-2” means that a provided compound inhibits BCL-2 in at least one assay described herein (e.g., biochemical or cellular). In some embodiments, the term “BCL-2 selective inhibitor” or “selectively inhibits BCL-2” means that a provided compound has the IC₅₀ for inhibiting the enzymes in BCL-2 family closely related to BCL-2 (such as BCL-XL) at least 5000 fold higher, at least 4000 fold higher, at least 3000 fold higher, at least 2000 fold higher, at least 1000 fold higher, at least 500 fold higher, at least 400 fold higher, at least 300 fold higher, at least 200 fold higher, at least 100 fold higher, at least 90 fold higher, at least 80 fold higher, at least 70 fold higher, at least 60 fold higher, at least 50 fold higher, at least 40 fold higher, at least 30 fold higher, at least 20 fold higher, at least 10 fold higher, than the IC₅₀ for inhibiting BCL-2.

In some embodiments, the compounds of the present disclosure are inhibitors of both BCL-2 and BCL-XL. For example, the compounds of the present disclosure have similar IC₅₀ values for inhibiting BCL-2 and BCL-XL in at least one assay described herein (e.g. biochemical or cellular). For example, the IC₅₀ values of the compounds of the present disclosure for inhibiting BCL-2 and BCL-XL are both within the range of 0-20 nM, or both within the range of 20-200 nM, or both within the range of 200-2000 nM.

In some embodiments, the compounds of the present disclosure do not significantly affect the activity of CYP2C9 enzyme. CYP2C9 enzyme is one of those commonly cytochromes P450 enzymes responsible for the metabolism of drugs. Without wishing to be bound by any particular theory, it is believed that CYP2C9 has a significant impact on drugs' pharmacokinetic properties and/or drug-drug interactions. In some embodiments, a compound of the present disclosure at 1 μM has an inhibition rate of less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1% against CYP2C9 enzyme, for example, as determined in the assay of the Examples of the present disclosure.

In some embodiments, compared to the previously reported BCL-2 inhibitors (such as Venetoclax), the compounds of the present disclosure have significantly reduced inhibition rate against CYP2C9 enzyme. Therefore, in one aspect, the compounds and pharmaceutically acceptable salts thereof provided herein show better profile against CYP2C9 than some known BCL-2 inhibitors (such as Venetoclax).

In some embodiments, the compounds of the present disclosure show good solubility in water. In some embodiments, the compounds of the present disclosure show a solubility in water of above 90 μM, above 100 μM, above 200 μM, above 30 μM, above 400 μM, above 500 μM, above 600 μM, above 700 μM, above 800 μM, above 900 μM, or above 1000 μM.

As a result of their BCL-2 or BCL-2/BCL-XL dual inhibitory activity (optionally selective BCL-2 inhibitory activity), the compounds of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof are useful in therapy, for example in the treatment of diseases, disorders or medical conditions mediated at least in part by BCL-2 or BCL-2/BCL-XL, including cancers.

As used herein, the term “cancer” is intended to encompass both non-metastatic cancer and metastatic cancer. In this context, treating cancer involves treatment of both primary tumors and tumor metastases.

As used herein, the term “therapy” is intended to have its normal meaning of dealing with a disease in order to entirely or partially relieve one, some or all of its symptoms, or to correct or compensate for the underlying pathology. The term “therapy” also includes “prophylaxis” unless there are specific indications to the contrary. The terms “therapeutic” and “therapeutically” should be interpreted in a corresponding manner.

As used herein, the term “prophylaxis” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment”, “treat” or “treating” is used synonymously with “therapy”. Similarly the term “treat” can be regarded as “applying therapy” where “therapy” is as defined herein.

Therefore, in one aspect, there is provided a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, for use in therapy.

In some embodiments, there is provided a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, for use as a medicament.

In some embodiments, there is provided a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, for use in the treatment of diseases, disorders or conditions. In some embodiments, the diseases, disorders or conditions are related to an increased level or activity of BCL-2 protein or BCL-2/BCL-XL proteins. In some embodiments, the disease, disorder or condition is selected from the group consisting of leukemia, Hodgkin lymphoma, Non-Hodgkin lymphoma, mantle cell lymphomas, gastro-intestinal cancer, gastric cancer, vascular cancer, biliary carcinomas, pancreatic cancer, colorectal cancer, esophageal cancer, hepatocellular cancer, melanoma, myeloma, oral cancer, ovarian cancer, small cell lung cancer, non-small cell lung cancer, myeloma, prostate cancer, bladder cancer, brain cancer, breast cancer, bone marrow cancer, cervical cancer and spleen cancer.

In some embodiments, the leukemia is selected from the group consisting of lymphatic leukemia, lymphocytic leukemia, chronic lymphocytic leukemia, small lymphocytic lymphoma, diffuse large B-cell lymphoma, acute myeloid leukemia, lymphoblastic leukemia, follicular lymphoma, lymphoid malignancies of T-cell or B-cell origin, myelogenous leukemia, granulocytic leukemia, polycythemia vera, erythremia.

In some embodiments, there is provided a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, for use in the manufacture of a medicament for the treatment of diseases, disorders or conditions. In some embodiments, the diseases, disorders or conditions are related to an increased level or activity of BCL-2 protein or BCL-2/BCL-XL proteins.

In some embodiments, there is provided a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, for use in the manufacture of a medicament for the treatment of a cancer.

### Pharmaceutical Compositions

The present disclosure provides pharmaceutical compositions comprising one or more compound of the present disclosure, or a pharmaceutically acceptable salt thereof. In some embodiments, the pharmaceutical composition comprises one or more compounds of the present disclosure, or a pharmaceutically acceptable salt thereof, and at least one pharmaceutical acceptable excipient.

A “pharmaceutical composition”, as used herein, is a formulation containing the compounds of the present disclosure in a form suitable for administration to a subject. In some embodiments, the pharmaceutical composition is in bulk or in unit dosage form. The unit dosage form is any of a variety of forms, including, for example, tablets, capsules, pills, powders, granules, sachets, cachets, lozenges, suspensions, emulsions, solutions, syrups, aerosols (as a solid or in a liquid medium), spray, ointment, paste, cream, lotion, gel, patch, inhalant, or suppository. The quantity of active ingredient (e.g., a formulation of the disclosed compound or salt, hydrate, solvate or isomer thereof) in a unit dose of composition is a therapeutically effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In some embodiments, the compound of the present disclosure is mixed under sterile conditions with a pharmaceutically acceptable excipient, and with any preservatives, buffers or propellants that are required.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, the term “therapeutically effective amount” refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

In some embodiments, the pharmaceutical compositions can be formulated so that a dosage of between 0.01-500 mg/kg body weight/day, for example, 0.05-500 mg/kg body weight/day, 0.1-500 mg/kg body weight/day, 0.1-400 mg/kg body weight/day, 0.1-300 mg/kg body weight/day, 0.1-200 mg/kg body weight/day, 0.1-100 mg/kg body weight/day, 0.1-80 mg/kg body weight/day, 1-100 mg/kg body weight/day or 1-80 mg/kg body weight/day of the compounds of the present disclosure, or a pharmaceutically acceptable salt thereof, can be administered.

In some embodiments, the pharmaceutical compositions comprise one or more compounds of the present disclosure, or a pharmaceutically acceptable salt thereof, as a first active ingredient, and further comprise a second active ingredient. The second active ingredient can be any anti-tumor agent known in the art, for example, antineoplastic agents, antiangiogenic agents, immunotherapy approaches, efficacy enhancers, and the like.

Examples of the antineoplastic agents include, but are not limited to, DNA alkylating agents (for example cisplatin, oxaliplatin, carboplatin, cyclophosphamide, nitrogen mustards like ifosfamide, bendamustine, melphalan, chlorambucil, busulphan, temozolamide and nitrosoureas like carmustine); antimetabolites (for example gemcitabine and antifolates such as fluoropyrimidines like 5-fluorouracil and tegafur, raltitrexed, methotrexate, cytosine arabinoside, and hydroxyurea); anti-tumor antibiotics (for example anthracyclines like adriamycin, bleomycin, doxorubicin, liposomal doxorubicin, pirarubicin, daunomycin, valrubicin, epirubicin, idarubicin, mitomycin, dactinomycin, amrubicin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, irinotecan, topotecan and camptothecin); inhibitors of DNA repair mechanisms such as CHK kinase; DNA-dependent protein kinase inhibitors; inhibitors of poly (ADP-ribose) polymerase (PARP inhibitors, including Olaparib, Rucaparib, Niraparib, Talazoparib, Pamiparib and Fluzoparib); and Hsp90 inhibitors such as tanespimycin and retaspimycin, inhibitors of ATR kinase (such as AZD6738); and inhibitors of WEE 1 kinase (such as AZD1775/MK-1775).

Examples of antiangiogenic agents include those that inhibit the effects of vascular endothelial growth factor, such as but not limited to, the anti-vascular endothelial cell growth factor antibody bevacizumab, a VEGF receptor tyrosine kinase inhibitor such as vandetanib (ZD6474), sorafenib, vatalanib (PTK787), sunitinib (SU11248), axitinib (AG-013736), pazopanib (GW 786034) and cediranib (AZD2171); compounds such as those disclosed in International Patent Applications WO 97/22596, WO 97/30035, WO 97/32856 and WO 98/13354; and compounds that work by other mechanisms (for example linomide, inhibitors of integrin avP3 function and angiostatin), or inhibitors of angiopoietins and their receptors (Tie-1 and Tie-2), inhibitors of PLGF, inhibitors of delta-like ligand (DLL-4).

Examples of immunotherapy approaches include, but are not limited to, ex-vivo and in-vivo approaches to increase the immunogenicity of patient tumor cells, such as transfection with cytokines such as interleukin 2, interleukin 4 or granulocyte-macrophage colony stimulating factor; approaches to decrease T-cell anergy or regulatory T-cell function; approaches that enhance T-cell responses to tumors, such as blocking antibodies to CTLA4 (for example ipilimumab and tremelimumab), B7H1, PD-1 (for example BMS-936558 or AMP-514), PD-L1 (for example MEDI4736) and agonist antibodies to CD 137; approaches using transfected immune cells such as cytokine-transfected dendritic cells; approaches using cytokine-transfected tumor cell lines, approaches using antibodies to tumor associated antigens, and antibodies that deplete target cell types (e.g., unconjugated anti-CD20 antibodies such as Rituximab, radiolabeled anti-CD20 antibodies Bexxar and Zevalin, and anti-CD54 antibody Campath); approaches using anti-idiotypic antibodies; approaches that enhance Natural Killer cell function; and approaches that utilize antibody-toxin conjugates (e.g. anti-CD33 antibody Mylotarg); immunotoxins such as moxetumumab pasudotox; agonists of toll-like receptor 7 or toll-like receptor 9.

Examples of efficacy enhancers include leucovorin.

Therefore, in some embodiments, there is provided pharmaceutical composition comprising a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, or a pharmaceutically acceptable salt thereof, and at least one additional anti-tumor agent. In some embodiments, there is one additional anti-tumor agent. In some embodiments, there are two additional anti-tumor agents. In some embodiments, there are three or more additional anti-tumor agents.

In some embodiments, the amount of additional anti-tumor agent present in the composition of the present disclosure can be no more than the amount that would normally be administered in a composition comprising that anti-tumor agent as the only active agent. In certain embodiments, the amount of the additional anti-tumor agent in the composition of the present disclosure will range from about 50% to 100% of the amount normally present in a composition comprising that anti-tumor agent as the only therapeutically active agent.

Therefore, in another aspect, there is provided a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof in combination with one or more anti-tumor agents listed above.

In some embodiments, the additional anti-tumor agent is selected from the group consisting of doxorubicin, irinotecan, topotecan, etoposide, mitomycin, bendamustine, chlorambucil, cyclophosphamide, ifosfamide, carmustine, melphalan and bleomycin.

As used herein, the term “combination” refers to simultaneous, separate or sequential administration. In some embodiments, “combination” refers to simultaneous administration. In some embodiments, “combination” refers to separate administration. In some embodiments, “combination” refers to sequential administration. Where the administration is sequential or separate, the delay in administering the second component should not be such as to lose the beneficial effect of the combination.

In a further aspect, there is provided a pharmaceutical composition comprising a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof in combination with one or more anti-tumor agents listed above, in association with a pharmaceutically acceptable excipient.

In a further aspect, there is provided a kit comprising a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof in combination with one or more anti-tumor agents listed above.

In a further aspect, there is provided a kit comprising:
(a) a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof in a first unit dosage form;
(b) an anti-tumor agent selected from those listed above in a second unit dosage form; and
(c) container for containing the first and second unit dosage forms.

### Methods for Treatment

In a further aspect, there is provided a method of treating BCL-2 or BCL-2/BCL-XL associated diseases, disorders or conditions in a subject in need thereof, which comprises administering to the subject a therapeutically effective amount of a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, or a pharmaceutical composition of the present disclosure, owning to the selective BCL-2 or BCL-2/BCL-XL inhibitory activity of the compounds of the present disclosure.

In some embodiments, the BCL-2 or BCL-2/BCL-XL associated disease, disorder or condition is cancer. In some embodiments, the cancer is selected from the group consisting of leukemia, Hodgkin lymphoma, Non-Hodgkin lymphoma, diffuse large B-cell lymphoma, chronic lymphocytic leukaemia, acute myeloid leukaemia, mantle cell lymphomas, gastro-intestinal cancer, gastric cancer, vascular cancer, biliary carcinomas, pancreatic cancer, colorectal cancer, esophageal cancer, hepatocellular cancer, melanoma, myeloma, oral cancer, ovarian cancer, small cell lung cancer, non-small cell lung cancer, myeloma, prostate cancer, bladder cancer, brain cancer, breast cancer, bone marrow cancer, cervical cancer, spleen cancer, glioblastoma, head and neck squamous cell carcinoma. In some embodiments, the cancer is head and neck squamous cell carcinoma, including but not limited to, lip carcinoma, oral cavity carcinoma, oropharynx carcinoma, hypopharynx carcinoma, glottic larynx carcinoma, supraglottic larynx carcinoma, ethmoid sinus carcinoma, maxillary sinus carcinoma, and occult primary carcinoma. In some embodiments, the cancer is leukemia, including but not limited to, lymphatic leukemia, lymphocytic leukemia, chronic lymphocytic leukemia, small lymphocytic lymphoma, diffuse large B-cell lymphoma, acute myeloid leukemia, lymphoblastic leukemia, follicular lymphoma, lymphoid malignancies of T-cell or B-cell origin, myelogenous leukemia, granulocytic leukemia, polycythemia vera, erythremia. In some embodiments, the cancer is metastatic cancer. In some embodiments, the metastatic cancer comprises metastases of the central nervous system. In some embodiments, the metastases of the central nervous system comprise brain metastases. In some embodiments, the metastases of the central nervous system comprise leptomeningeal metastases. “Leptomeningeal metastases” occur when cancer spreads to the meninges, the layers of tissue that cover the brain and the spinal cord. Metastases can spread to the meninges through the blood or they can travel from brain metastases, carried by the cerebrospinal fluid (CSF) that flows through the meninges.

As used herein, the term “subject in need thereof” is a subject having a BCL-2 or BCL-2/BCL-XL associated disease, disorder or condition (e.g., cancer), or a subject having an increased risk of developing BCL-2 or BCL-2/BCL-XL associated disease, disorder or condition (e.g., cancer) relative to the population at large. In the case of cancer, a subject in need thereof can have a precancerous condition. A “subject” includes a warm-blooded animal. In some embodiments, the warm-blooded animal is a mammal, e.g. human.

In this context, the term “therapeutically effective amount” refers to an amount of a compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof which is effective to provide “therapy” in a subject, or to “treat” a BCL-2 or BCL-2/BCL-XL associated disease, disorder or condition in a subject. In the case of cancer, the therapeutically effective amount may cause any of the changes observable or measurable in a subject as described in the definition of “therapy”, “treatment” and “prophylaxis” above. For example, the effective amount can reduce the number of cancer or tumor cells; reduce the overall tumor size; inhibit or stop tumor cell infiltration into peripheral organs including, for example, the soft tissue and bone; inhibit and stop tumor metastasis; inhibit and stop tumor growth; relieve to some extent one or more of the symptoms associated with the cancer; reduce morbidity and mortality; improve quality of life; or a combination of such effects. An effective amount may be an amount sufficient to decrease the symptoms of a disease responsive to inhibition of BCL-2 or BCL-2/BCL-XL. For cancer therapy, efficacy in-vivo can, for example, be measured by assessing the duration of survival, time to disease progression (TTP), the response rates (RR), duration of response, and/or quality of life. As recognized by those skilled in the art, effective amounts may vary depending on route of administration, excipient usage, and co-usage with other agents. For example, where a combination therapy is used, the amount of the compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof described in this specification and the amount of the other pharmaceutically active agent(s) are, when combined, jointly effective to treat a targeted disorder in the animal patient. In this context, the combined amounts are in a “therapeutically effective amount” if they are, when combined, sufficient to decrease the symptoms of a disease responsive to inhibition of BCL-2 or BCL-2/BCL-XL as described above.

In generally, “therapeutically effective amount” may be determined by one skilled in the art by, for example, starting with the dosage range described in this specification for the compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof and an approved or otherwise published dosage range(s) of the other pharmaceutically active compound(s).

The method of treating BCL-2 or BCL-2/BCL-XL associated diseases, disorders or conditions described in this specification may be used as a monotherapy. As used herein, the term “monotherapy” refers to the administration of a single active or therapeutic compound to a subject in need thereof. In some embodiments, monotherapy will involve administration of a therapeutically effective amount of one of the compounds of the present disclosure, or a tautomer, a stereoisomer, or a pharmaceutically acceptable salt thereof, to a subject in need of such treatment.

Depending upon the particular diseases or conditions to be treated, the method of treating BCL-2 or BCL-2/BCL-XL associated diseases, disorders or conditions described in this specification may involve, in addition to administration of the compound of the present disclosure, one or more additional therapies, for example, conventional surgery, radiotherapy, chemotherapy, immunotherapy, or a combination of such additional therapies. As used herein, the term “combination therapy” refers to the administration of a combination of multiple active compounds.

The additional therapies, such as additional anti-tumor agents, may be administered separately from the compounds of the present disclosure, as part of a multiple dosage regimen. Alternatively, these additional therapies may be part of a single dosage form, mixed with the compounds of the present disclosure in a single composition.

In some embodiments, the compounds of the present disclosure may be administered simultaneously, sequentially or separately to treatment with the conventional surgery, radiotherapy, chemotherapy or immunotherapy.

Radiotherapy may include one or more of the following categories of therapy: (i) external radiation therapy using electromagnetic radiation, and intraoperative radiation therapy using electromagnetic radiation; (ii) internal radiation therapy or brachytherapy; including interstitial radiation therapy or intraluminal radiation therapy; or (iii) systemic radiation therapy, including but not limited to iodine 131 and strontium 89.

Chemotherapy may include anti-tumor agents known in the art, for example, antineoplastic agents, antiangiogenic agents, efficacy enhancers, and the like described in this specification.

Immunotherapy may include, for example, immune checkpoint modulator. Immune checkpoints are regulators of the immune system, and belong to immunoinhibitory pathway or immunostimulatory pathway, responsible for co-stimulatory or inhibitory interactions of T-cell responses, and regulate and maintain self-tolerance and physiological immune responses. Non-limiting immunoinhibitory checkpoint molecules found in the immunoinhibitory pathways can include LAG3 (CD223), A2AR, B7-H3 (CD276), B7-H4 (VTCN1), BTLA (CD272), BTLA, CD160, CTLA-4 (CD152), IDO1, ID02, TDO, KIR, LAIR-1, NOX2, PD-1, PD-L1, PD-L2, TIM-3, VISTA, SIGLEC-7 (CD328), TIGIT, PVR (CD155), TGFβ, or SIGLEC9 (CD329), among others. Non-limiting immunostimulatory checkpoint molecules found in the immunostimulatory pathways can include CD2, CD3, CD7, CD16, CD27, CD30, CD70, CD83, CD28, CD80 (B7-1), CD86 (B7-2), CD40, CD40L (CD154), CD47, CD122, CD137, CD137L, OX40 (CD134), OX40L (CD252), NKG2C, 4-1BB, LIGHT, PVRIG, SLAMF7, HVEM, BAFFR, ICAM-1, 2B4, LFA-1, GITR, ICOS (CD278), or ICOSLG (CD275), among others.

Therefore, in one aspect, there is provided a method of treating BCL-2 or BCL-2/BCL-XL associated diseases, disorders or conditions in a subject in need thereof, wherein the compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof is administered simultaneously, separately or sequentially with a second therapy.

In some embodiments, the second therapy is chemotherapy or immunotherapy. In some embodiments, the second therapy is selected from the group consisting of a chemotherapeutic agent, an anti-tumor agent, a radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, and cytokines. In some embodiments, the second therapy is a BTK inhibitor, a BCR-ABL inhibitor, a JAK3 inhibitor, or a PARP inhibitor.

In another aspect, there is provided a method of treating BCL-2 or BCL-2/BCL-XL associated diseases, disorders or conditions in a subject in need thereof, wherein the compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof is administered simultaneously, separately or sequentially with one or more additional anti-tumor agents.

In some embodiments, the BCL-2 or BCL-2/BCL-XL associated disease, disorder or condition is cancer. In certain embodiments, the amounts of the compound of Formula I, Formula II, Formula III, Formula IV, Formula IV(a), Formula IV(b), Formula IV(c), Formula IV(d), Formula IV(e), or a tautomer, a stereoisomer, or a pharmaceutically acceptable salt thereof, and the one or more additional anti-tumor agents are jointly effective in producing an anti-cancer effect.

In some embodiments, the additional anti-tumor agent includes antineoplastic agents, antiangiogenic agents, immunotherapy approaches, efficacy enhancers and the like.

In some embodiments, the additional anti-tumor agent is selected from the group consisting of doxorubicin, irinotecan, topotecan, etoposide, mitomycin, bendamustine, chlorambucil, cyclophosphamide, ifosfamide, carmustine, melphalan and bleomycin.

In some embodiments, the compounds of the present disclosure may be administered simultaneously, sequentially or separately with antineoplastic agents.

### EXAMPLES

For the purpose of illustration, the following examples are included. However, it is to be understood that these examples do not limit the invention and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the present disclosure.

For illustrative purposes, the following shows general synthetic schemes for preparing the compounds of the present disclosure as well as key intermediates. Those skilled in the art will appreciate that other synthetic schemes may be used to synthesize the inventive compounds. Although specific starting materials and reagents are depicted in the General Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

The following abbreviations have been used in the examples:

| | |
|---|---|
| DMF | N,N-dimethylformamide |
| rt | room temperature |
| THF | tetrahydrofuran |
| SEMCl | 2-(Trimethylsilyl)ethoxymethyl chloride |
| EA | ethyl acetate |
| PE | petroleum ether |
| EtOAc | ethyl acetate |
| BINAP | 2,2′-Bis(diphenylphosphino)-1,1′-binaphthalene |
| DCM | dichloromethane |
| TFA | trifluoroacetic acid |
| DPPE | 1,2-bis(diphenylphosphino) ethane |
| NMP | 1-methyl-2-pyrrolidone |
| EDCI | 1-Ethyl-3-(3-dimethylaminopropyl)carbodiimide |
| DMAP | 4-(Dimethylamino)pyridine |
| DIPEA | N,N-Diisopropylethylamine |
| eq | equivalent(s) |
| aq | aqueous |

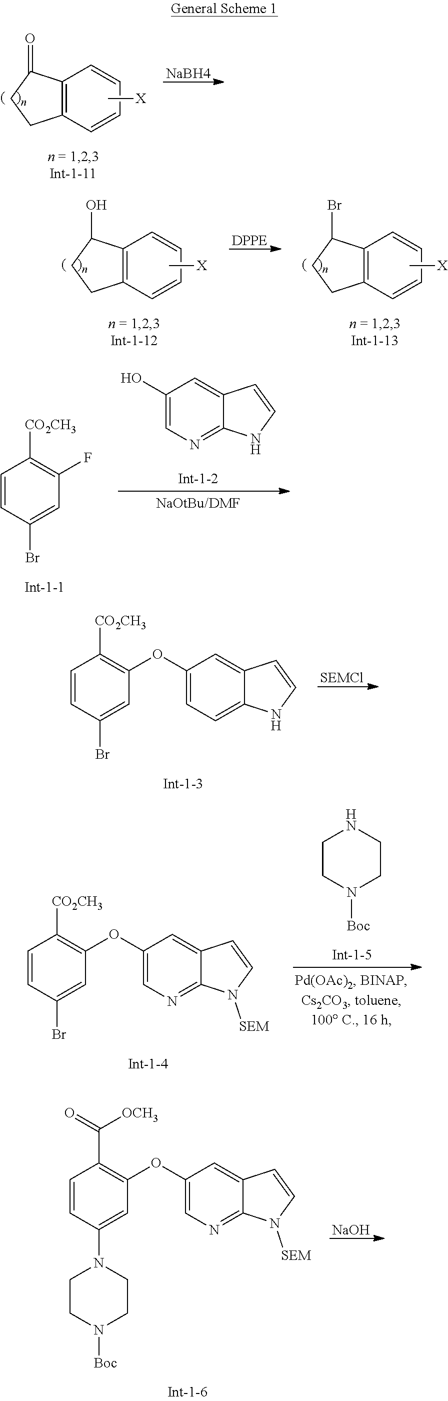

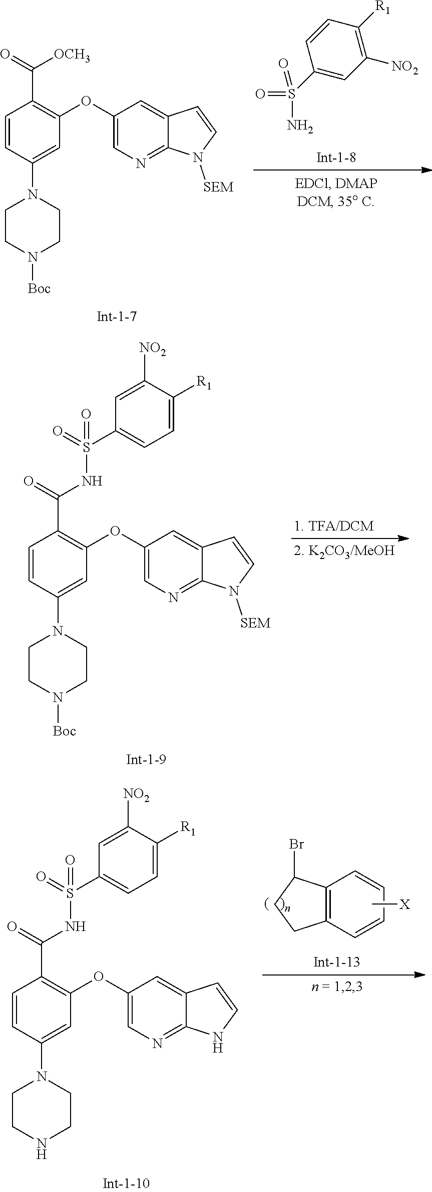

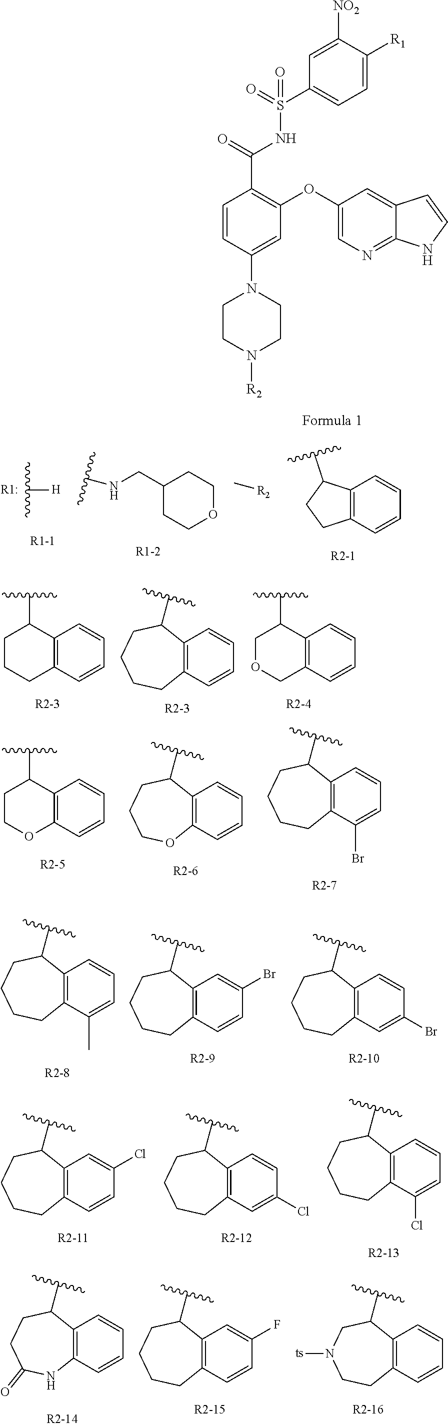

### General Step 1: Synthesis of Int-1-3

NaOtBu (9.1 g, 94.8 mmol) was resolved in DMF (50 mL). It was added slowly to a solution of Int-1-1 (20 g, 86.2 mmol), Int-1-2 (10.3 g, 77.5 mmol) in DMF (50 mL) at 5° C. Then the mixture was warmed to rt for 16 h. Water (200 mL) was added. The crude product was collected by filtration. The collected product was dried, then re-crystallization with EA and heptane to give 20 g Int-1-3 (73%). LCMS: [M+H]⁺=347.1.

### General Step 2: Synthesis of Int-1-4

To a stirred solution of Int-1-3 (10.00 g, 28.91 mmol) in THE (50 mL) was added NaH (1.70 g, 43.31 mmol). The mixture was stirred for 1 h and the SEMCl (6.62 g, 37.5 mmol) was added. The mixture was stirred for 2 h at rt and LC-MS showed the total consumption of Int-1-3. It was quenched by water (20 mL) and extracted with EA (3×100 mL). The combined organic layer was washed with brine (1×50 mL), dried it over sodium sulfate and concentrated under reduced pressure. The residue was purified by column chromatography (PE:EtOAc=4:1, at 254 nm) to give Int-1-4 (13 g, 94%). LCMS: [M+H]⁺=477.1.

### General Step 3: Synthesis of Int-1-6

To a solution of Int-1-4 (5.0 g, 10.5 mmol) and Int-1-5 (2.1 g, 11.5 mmol) in anhydrous toluene (100 mL) was added Pd(OAc)₂ (235.0 mg, 0.11 mmol), BINAP (9.7 g, 15.72 mmol), and Cs₂CO₃ (6.8 g, 20.8 mmol) at rt. The reaction mixture was stirred at 110° C. for 6 h under N₂ atmosphere. LC-MS showed the reaction was completed. Toluene was removed under reduced pressure to give crude product. The crude product was purified by combine flash (PE:EtOAc=4:1, at 254 nm) to give Int-1-6 (3 g, 49%). LCMS: [M+H]⁺: 583.3.

### General Step 4: Synthesis of Int-1-7

To a solution of Int-1-6 (3.0 g, 5.2 mmol) in MeOH (50 mL) was added NaOH (1.1 g, 26.0 mmol) solution (H₂O, 10 mL). The reaction mixture was stirred at 60° C. for 6 hours. LC-MS showed the reaction was completed. The residue was treated with aqueous HCl (1N, 5 mL) to pH=5. The reaction mixture was poured into water (100 mL) and extracted with EtOAc (3×25 mL). The combined extracts were washed with brine (2×50 mL), dried over Na₂SO₄, concentrated to give 2.5 g Int-1-7. LCMS: [M+H]⁺: 569.3.

### General Step 5: Synthesis of Int-1-9

To a solution of Int-1-7 (1 eq) and Int-1-8 (1.2 eq) in anhydrous DCM (50 mL) was added EDCI (1.5 eq), DMAP (1.5 eq), and DIPEA (1.5 eq) at rt. The reaction mixture was stirred at 35° C. for 16 h under N₂ atmosphere. LC-MS showed the reaction was completed. The reaction mixture was poured into EtOAc (30 mL) and washed with H₂O (2×25 mL). The extract was washed with brine (1×20 mL), dried over Na₂SO₄, concentrated to afford crude product. The crude product was purified by combine flash (PE:EtOAc=1:1, at 254 nm) to give Int-1-9.

### General Step 6: Synthesis of Int-1-10

To a solution of Int-1-9 (1 eq) in anhydrous DCM (5 mL) was added TFA (1.5 mL) at rt. The reaction mixture was stirred at rt for 2 h under N₂ atmosphere. LC-MS showed the reaction was completed. The reaction mixture was concentrated. MeOH (20 mL) was added followed by K₂CO₃ (10 eq) in H₂O (5 mL) at rt. The reaction mixture was stirred at rt for 16 h under N₂ atmosphere. LC-MS showed the reaction was completed. The MeOH was removed under reduced pressure, 50 mL DCM was added to the mixture, dried over Na₂SO₄, concentrated to afford crude product. The crude product was purified by TLC (DCM:MeOH=10:1, at 254 nm) to give Int-1-10.

### General Step 7: Synthesis of Int-1-12

To a stirred solution of Int-1-11 (1 eq) in MeOH (50 mL) was added NaBH₄ (1.5 eq). The resulting mixture was stirred at rt for 6 h. The reaction mixture was diluted with water (200 mL) and was extracted with EtOAc (3×100 mL). The combined organic extracts were concentrated. The residue was purified by combine flash (0 to 40% PE in EtOAc) to give Int-1-12.

### General Step 8: Synthesis of Int-1-13

Br₂ (1.5 eq) was added to a solution of DPPE (0.60 eq) in 100 ml DCM and stirred at −20° C. for 1 h. Then Int-1-12 (1 eq) was added into the mixture. The resulting mixture was stirred at 0° C. for 5 h. Hexane (100 mL) was added and filtered. Organic layer was dried and was removed in vacuo. The residue was purified by combine flash (0 to 40% PE in EtOAc) to give Int-1-13.

### General Step 9: Synthesis of Formula 1

To a stirred solution of Int-1-10 (1 eq) and DIEA (2.5 eq) in anhydrous NMP (2 mL) was added Int-1-13 (2.5 eq). The mixture was stirred at 35° C. for 16 h. The reaction mixture was concentrated, and the residue was dissolved in DCM (50 mL). The organic layer was washed with water (30 mL), dried over anhydrous Na₂SO₄ and evaporated in vacuo. The residue was purified by prep-HPLC to give Formula 1.

### Example 1: Synthesis of Compound 1

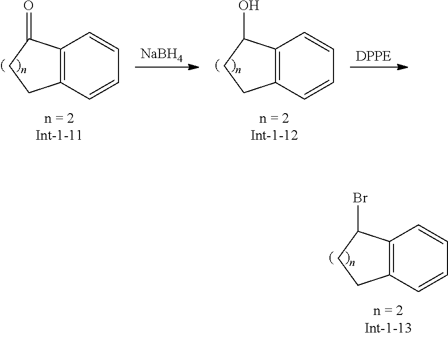

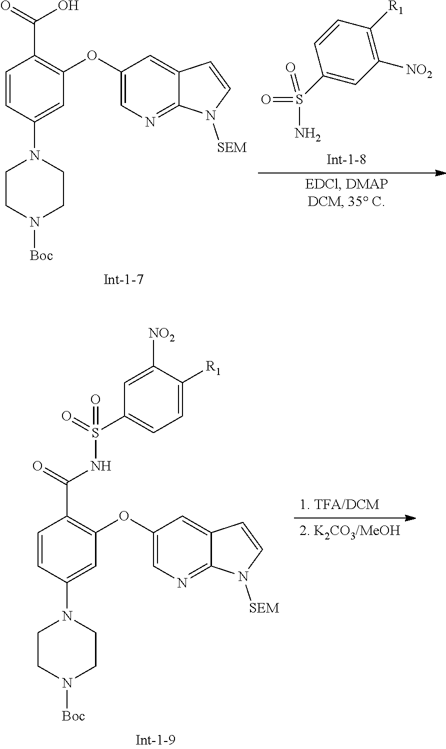

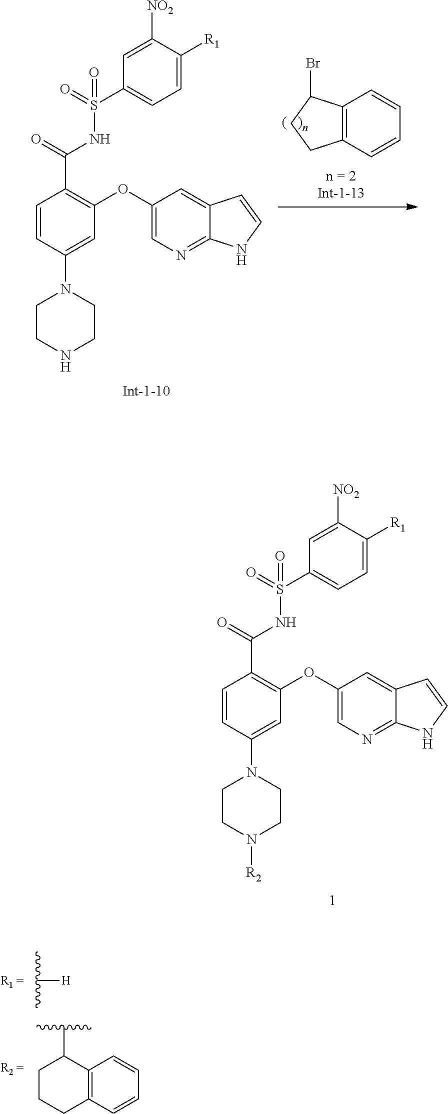

### Step 1: Synthesis of Int-1-9

To a solution of Int-1-7 (1.0 g, 1.76 mmol) and Int-1-8 (480.0 mg, 2.2 mmol) in anhydrous DCM (50 mL) was added EDCI (500 mg, 2.64 mmol), DMAP (322 mg, 2.64 mmol), and DIPEA (340 mg, 2.64 mmol) at rt. The reaction mixture was stirred at 35° C. for 16 h under N₂ atmosphere. LC-MS showed the reaction was completed. The reaction mixture was poured into EtOAc (30 mL) and washed with H₂O (2×25 mL). The extract was washed with brine (1×20 mL), dried over Na₂SO₄, concentrated to afford crude product. The crude product was purified by combine flash (PE:EtOAc=1:1, at 254 nm) to give Int-1-9 (480 mg, 37%). LCMS: [M+H]⁺: 753.2.

### Step 2: Synthesis of Int-1-10

To a solution of Int-1-9 (480 mg, 0.64 mmol) in anhydrous DCM (5 mL) was added TFA (1.5 mL) at rt. The reaction mixture was stirred at rt for 2 h under N₂ atmosphere. LC-MS showed the reaction was completed. The reaction mixture was concentrated. MeOH (20 mL) was added followed by K₂CO₃ (1 g, 7.2 mmol) in H₂O (5 mL) at rt. The reaction mixture was stirred at rt for 16 h under N₂ atmosphere. LC-MS showed the reaction was completed. The MeOH was removed under reduced pressure, 50 mL DCM was added to the mixture, dried over Na₂SO₄, concentrated to afford crude product. The crude product was purified by TLC (DCM:MeOH=10:1, at 254 nm) to give Int-1-10 (230 mg, 69%). LCMS: [M+H]⁺: 523.1.

### Step 3: Synthesis of Int-1-12

To a stirred solution of Int-1-11 (5.0 g, 34.2 mmol) in MeOH (50 mL) was added NaBH₄ (1.90 g, 51.3 mmol). The resulting mixture was stirred at rt for 6 h. The reaction mixture was diluted with water (200 mL) and was extracted with EtOAc (3×100 mL). The combined organic extracts were concentrated. The residue was purified by combine flash (0 to 40% PE in EtOAc) to give Int-1-12 (4.5 g, 90%). LCMS: [M−17]⁺: 131.

### Step 4: Synthesis of Int-1-13

Br₂ (800 mg, 5 mmol) was added to a solution of DPPE (800 mg, 2 mmol) in 100 ml DCM and stirred at −20° C. for 1 h. Then Int-1-12 (500 mg, 3.37 mmol) was added into the mixture. The resulting mixture was stirred at 0° C. for 5 h. Hexane (100 mL) was added and filtered. Organic layer was dried and was removed in vacuo. The residue was purified by combine flash (0 to 40% PE in EtOAc) to give Int-1-13 (400 mg, 57%).

### Step 5: Synthesis of Compound 1

To a stirred solution of Int-1-10 (50 mg, 0.08 mmol) and DIEA (30 mg, 0.23 mmol) in anhydrous NMP (2 ml) was added Int-1-13 (40 mg, 0.18 mmol). The mixture was stirred at 35° C. for 16 h. The reaction mixture was concentrated, and the residue was dissolved in DCM (50 mL). The organic layer was washed with water (30 mL), dried over anhydrous Na₂SO₄ and evaporated in vacuo. The residue was purified by prep-HPLC to give Compound 1 (15 mg, 29%). LCMS [M+H]⁺=653.2.

### Example 2: Synthesis of Compound 27

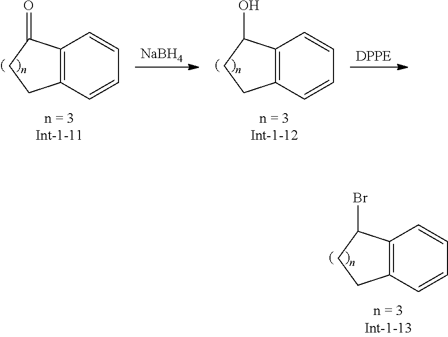

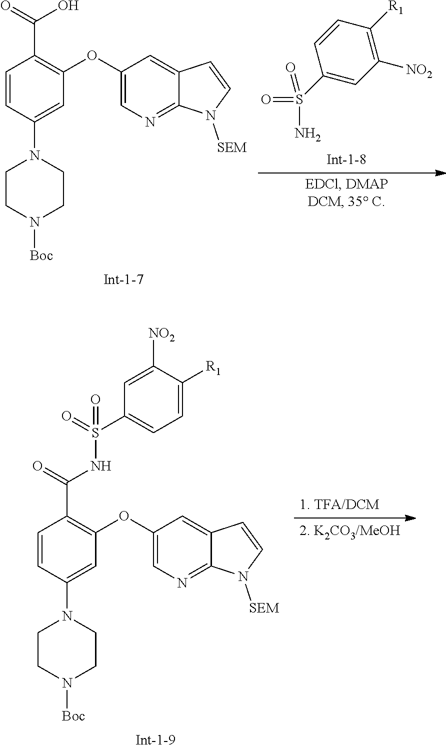

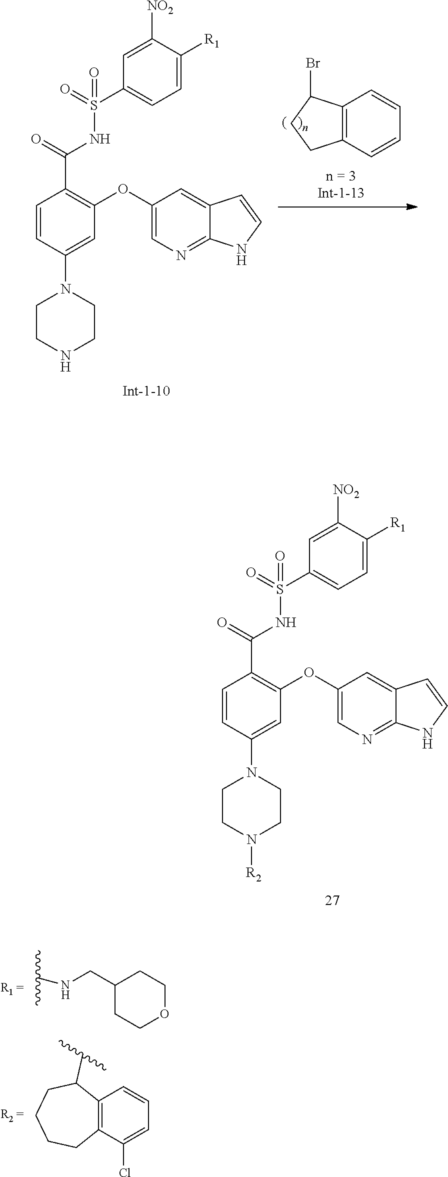

### Step 1: Synthesis of Int-1-9

To a solution of Int-1-7 (1.0 g, 1.76 mmol) and Int-1-8 (693 mg, 2.2 mmol) in anhydrous DCM (50 mL) was added EDCI (500 mg, 2.64 mmol), DMAP (322 mg, 2.64 mmol), and DIPEA (340 mg, 2.64 mmol) at rt. The reaction mixture was stirred at 35° C. for 16 h under N₂ atmosphere. LC-MS showed the reaction was completed. The reaction mixture was poured into EtOAc (30 mL) and washed with H₂O (2×25 mL). The extract was washed with brine (1×20 mL), dried over Na₂SO₄, concentrated to afford crude product. The crude product was purified by combine flash (PE:EtOAc=1:1, at 254 nm) to give Int-1-9. (530 mg, 40%). LCMS: [M+H]⁺: 865.9

### Step 2: Synthesis of Int-1-10

To a solution of Int-1-9 (530 mg, 0.7 mmol) in anhydrous DCM (5 mL) was added TFA (1.5 mL) at rt. The reaction mixture was stirred at rt for 2 h under N₂ atmosphere. LC-MS showed the reaction was completed. The reaction mixture was concentrated. MeOH (20 mL) was added followed by K₂CO₃ (1 g, 7.2 mmol) in H₂O (5 mL) at rt. The reaction mixture was stirred at rt for 16 h under N₂ atmosphere. LC-MS showed the reaction was completed. The MeOH was removed under reduced pressure, 50 ml DCM was added to the mixture, dried over Na₂SO₄, concentrated to afford crude product. The crude product was purified by TLC (DCM:MeOH=10:1, at 254 nm) to give Int-1-10 (330 mg, 70%). LCMS: [M+H]⁺: 636.2.

### Step 3: Synthesis of Int-1-12

To a stirred solution of Int-1-11 (400 mg, 2.06 mmol) in MeOH (20 mL) was added NaBH₄ (118 mg, 3.09 mmol). The resulting mixture was stirred at rt for 6 h. The reaction mixture was diluted with water (200 mL) and was extracted with EtOAc (100 mL×3). The combined organic extracts were concentrated. The residue was purified by combine flash (0 to 40% PE in EtOAc) to give Int-1-12 (390 g, 97%). LCMS: [M−17]⁺: 165.

### Step 4: Synthesis of Int-1-13

Br₂ (480 mg, 3 mmol) was added to a solution of DPPE (477 mg, 1.2 mmol) in 20 ml DCM and stirred at −20° C. for 1 h. Then Int-1-12 (390 mg, 2 mmol) was added into the mixture. The resulting mixture was stirred at 0° C. for 5 h. Hexane (100 mL) was added and filtered. Organic layer was dried and was removed in vacuo. The residue was purified by combine flash (0 to 40% PE in EtOAc) to give Int-1-13 (450 mg, 82%).

### Step 5: Synthesis of Compound 27

To a stirred solution of Int-1-10 (40 mg, 0.06 mmol) and DIEA (20 mg, 0.15 mmol) in anhydrous NMP (2 ml) was added Int-1-13 (39 mg, 0.15 mmol). The mixture was stirred at 35° C. for 16 h. The reaction mixture was concentrated, and the residue was dissolved in DCM (50 mL). The organic layer was washed with water (30 mL), dried over anhydrous Na₂SO₄ and evaporated in vacuo. The residue was purified by prep-HPLC to give Compound 27 (6 mg, 12%). LCMS [M+H]⁺=814.2.

The following compounds were prepared according to the above described methods using different starting materials.

| Compound | | MS(ESI): m/z |
|---|---|---|
| No. | IUPAC Name | (M + H⁺) |
| 2 | 2-((1H-pyrrolo[2,3-b]pyridin-5-yl)oxy)-N-((3-nitro- | 766.4 |
| | 4-(((tetrahydro-2H-pyran-4- | |
| | yl)methyl)amino)phenyl)sulfonyl)-4-(4-(1,2,3,4- | |
| | tetrahydronaphthalen-1-yl)piperazin-1- | |
| | yl)benzamide | |
| 3 | 2-((1H-pyrrolo[2,3-b]pyridin-5-yl)oxy)-4-(4-(2,3- | 639.2 |
| | dihydro-1H-inden-1-yl)piperazin-1-yl)-N-((3- | |
| | nitrophenyl)sulfonyl)benzamide | |
| 4 | 2-((1H-pyrrolo[2,3-b]pyridin-5-yl)oxy)-4-(4-(2,3- | 752.3 |
| | dihydro-1H-inden-1-yl)piperazin-1-yl)-N-((3-nitro- | |
| | 4-(((tetrahydro-2H-pyran-4- | |
| | yl)methyl)amino)phenyl)sulfonyl)benzamide | |
| 5 | 2-((1H-pyrrolo[2,3-b]pyridin-5-yl)oxy)-N-((3- | 667.3 |
| | nitrophenyl)sulfonyl)-4-(4-(6,7,8,9-tetrahydro-5H- | |
| | benzo[7]annulen-5-yl)piperazin-1-yl)benzamide | |
| 6 | 2-((1H-pyrrolo[2,3-b]pyridin-5-yl)oxy)-N-((3-nitro- | 780.3 |
| | 4-(((tetrahydro-2H-pyran-4- | |
| | yl)methyl)amino)phenyl)sulfonyl)-4-(4-(6,7,8,9- | |
| | tetrahydro-5H-benzo[7]annulen-5-yl)piperazin-1- | |
| | yl)benzamide | |
| 9 | 2-((1H-pyrrolo[2,3-b]pyridin-5-yl)oxy)-4-(4- | 655.2 |
| | (chroman-4-yl)piperazin-1-yl)-N-((3- | |
| | nitrophenyl)sulfonyl)benzamide | |
| 10 | 2-((1H-pyrrolo[2,3-b]pyridin-5-yl)oxy)-4-(4- | 655.2 |
| | (isochroman-4-yl)piperazin-1-yl)-N-((3- | |
| | nitrophenyl)sulfonyl)benzamide | |
| 12 | 2-((1H-pyrrolo[2,3-b]pyridin-5-yl)oxy)-4-(4- | 768.0 |
| | (isochroman-4-yl)piperazin-1-yl)-N-((3-nitro-4- | |
| | (((tetrahydro-2H-pyran-4- | |
| | yl)methyl)amino)phenyl)sulfonyl)benzamide | |
| 17 | 2-((1H-pyrrolo[2,3-b]pyridin-5-yl)oxy)-4-(4- | 768.3 |
| | (chroman-4-yl)piperazin-1-yl)-N-((3-nitro-4- | |
| | (((tetrahydro-2H-pyran-4- | |
| | yl)methyl)amino)phenyl)sulfonyl)benzamide | |
| 18 | 2-((1H-pyrrolo[2,3-b]pyridin-5-yl)oxy)-4-(4-(1- | 860.2 |
| | bromo-6,7,8,9-tetrahydro-5H-benzo[7]annulen-5- | |
| | yl)piperazin-1-yl)-N-((3-nitro-4-(((tetrahydro-2H- | |
| | pyran-4- | |
| | yl)methyl)amino)phenyl)sulfonyl)benzamide | |
| 20 | 2-((1H-pyrrolo[2,3-b]pyridin-5-yl)oxy)-4-(4-(1- | 794.3 |
| | methyl-6,7,8,9-tetrahydro-5H-benzo[7]annulen-5- | |
| | yl)piperazin-1-yl)-N-((3-nitro-4-(((tetrahydro-2H- | |
| | pyran-4- | |
| | yl)methyl)amino)phenyl)sulfonyl)benzamide | |
| 21 | 2-((1H-pyrrolo[2,3-b]pyridin-5-yl)oxy)-4-(4-(3- | 858.0 |
| | bromo-6,7,8,9-tetrahydro-5H-benzo[7]annulen-5- | |
| | yl)piperazin-1-yl)-N-((3-nitro-4-(((tetrahydro-2H- | |
| | pyran-4- | |
| | yl)methyl)amino)phenyl)sulfonyl)benzamide | |
| 23 | 2-((1H-pyrrolo[2,3-b]pyridin-5-yl)oxy)-4-(4-(2- | 858.0 |
| | bromo-6,7,8,9-tetrahydro-5H-benzo[7]annulen-5- | |
| | yl)piperazin-1-yl)-N-((3-nitro-4-(((tetrahydro-2H- | |
| | pyran-4- | |
| | yl)methyl)amino)phenyl)sulfonyl)benzamide | |
| 25 | 2-((1H-pyrrolo[2,3-b]pyridin-5-yl)oxy)-4-(4-(2- | 814.0 |
| | chloro-6,7,8,9-tetrahydro-5H-benzo[7]annulen-5- | |
| | yl)piperazin-1-yl)-N-((3-nitro-4-(((tetrahydro-2H- | |
| | pyran-4- | |
| | yl)methyl)amino)phenyl)sulfonyl)benzamide | |
| 26 | 2-((1H-pyrrolo[2,3-b]pyridin-5-yl)oxy)-4-(4-(3- | 814.3 |
| | chloro-6,7,8,9-tetrahydro-5H-benzo[7]annulen-5- | |
| | yl)piperazin-1-yl)-N-((3-nitro-4-(((tetrahydro-2H- | |
| | pyran-4- | |
| | yl)methyl)amino)phenyl)sulfonyl)benzamide | |
| 28 | 2-((1H-pyrrolo[2,3-b]pyridin-5-yl)oxy)-N-((3-nitro- | 795.2 |
| | 4-(((tetrahydro-2H-pyran-4- | |
| | yl)methyl)amino)phenyl)sulfonyl)-4-(4-(2-oxo- | |
| | 2,3,4,5-tetrahydro-1H-benzo[b]azepin-5- | |
| | yl)piperazin-1-yl)benzamide | |
| 37 | 2-((1H-pyrrolo[2,3-b]pyridin-5-yl)oxy)-4-(4-(3- | 798.2 |
| | fluoro-6,7,8,9-tetrahydro-5H-benzo[7]annulen-5- | |
| | yl)piperazin-1-yl)-N-((3-nitro-4-(((tetrahydro-2H- | |
| | pyran-4- | |
| | yl)methyl)amino)phenyl)sulfonyl)benzamide | |
| 35 | 2-((1H-pyrrolo[2,3-b]pyridin-5-yl)oxy)-N-((3-nitro- | 935.1 |
| | 4-(((tetrahydro-2H-pyran-4- | |
| | yl)methyl)amino)phenyl)sulfonyl)-4-(4-(3-tosyl- | |
| | 2,3,4,5-tetrahydro-1H-benzo[d]azepin-1- | |
| | yl)piperazin-1-yl)benzamide | |

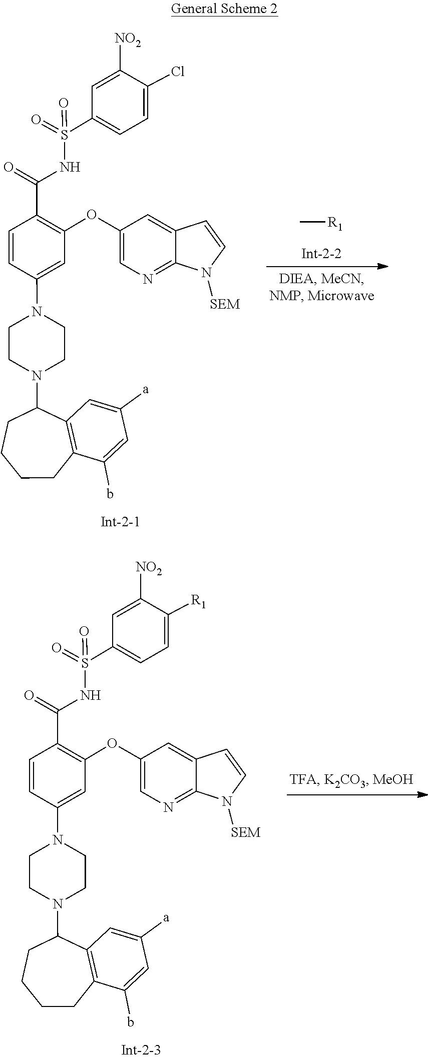

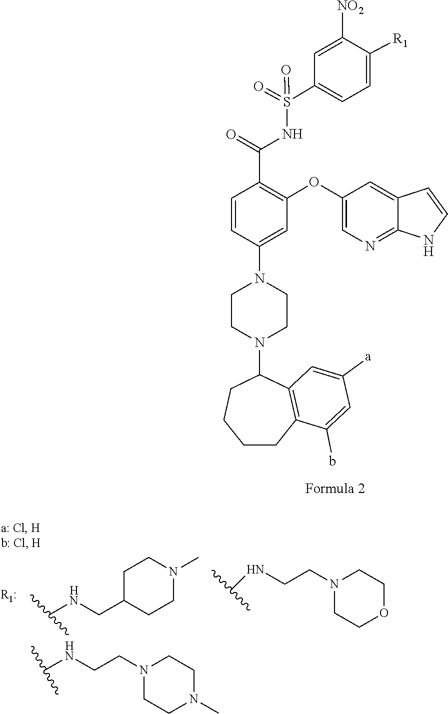

### General Step 1: Synthesis of Int-2-3

To a stirred solution of Int-2-1 (1 eq) and Int-2-2 (5 eq) in NMP (3 mL) was added DIEA (2 mL). The mixture was stirred at 90° C. for 2 h under microwave. The reaction was purified by prep-HPLC (DCM:MeOH=15:1) to give Int-2-3.

### General Step 2: Synthesis of Formula 2

To a solution of Int-2-3 (1 eq) in anhydrous DCM (5 mL) was added TFA (0.5 mL) at rt. The reaction mixture was stirred at rt for 2 h under N₂ atmosphere. LC-MS showed the reaction was completed. The reaction mixture was concentrated. MeOH (10 mL) was added followed by K₂CO₃ (10 eq) in H₂O (2 mL) at rt. The reaction mixture was stirred at rt for 16 h under N₂ atmosphere. LC-MS showed the reaction was completed. The MeOH was removed under reduced pressure, 50 mL DCM was added to the mixture, dried over Na₂SO₄, concentrated to afford crude product. The crude product was purified by TLC (DCM:MeOH=10:1, at 254 nm) to give Formula 2.

### Example 3: Synthesis of Compound 29

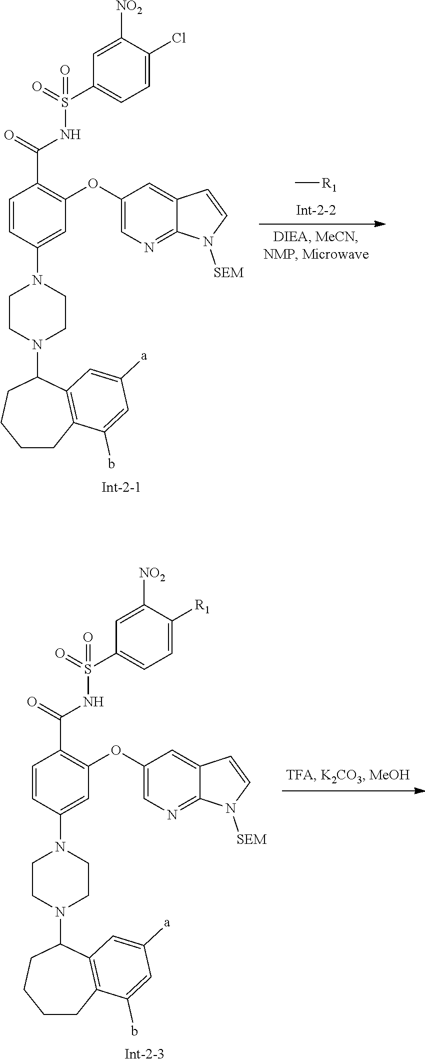

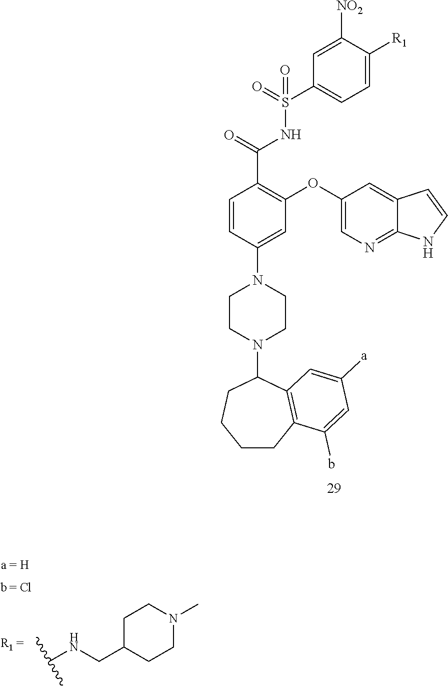

### Step 1: Synthesis of Int-2-3

To a solution of Int-2-1 (50 mg, 0.058 mmol) and (1-methylpiperidin-4-yl) methanamine (44 mg, 0.35 mmol) in anhydrous NMP (2 mL) was added DIPEA (1 mL) at rt. The reaction mixture was stirred at microwave at 120° C. for 2 h under N₂ atmosphere. LC-MS showed the reaction was completed. The reaction mixture was poured into EtOAc (30 mL) and washed with brine (2×20 mL), dried over Na₂SO₄, concentrated to afford 100 mg of crude product. The crude product was purified by TLC (DCM:MeOH=10:1, at 254 nm) to give Int-2-3 (35 mg). LCMS: [M+H]⁺: 960.3.

### Step 2: Synthesis of Compound 29

To a solution of Int-2-3 (35 mg, 0.035 mmol) in anhydrous DCM (3 mL) was added TFA (1 mL) at rt. The reaction mixture was stirred at rt for 2 h under N₂ atmosphere. MeOH (3 mL) was added K₂CO₃ in H₂O (1 mL) at rt. The reaction mixture was stirred at rt for 16 h under N₂ atmosphere. LC-MS showed the reaction was completed. The MeOH was removed under reduced pressure, 50 ml DCM was added to the mixture, dried over Na₂SO₄, concentrated to afford crude product. The crude product was purified by TLC (DCM:MeOH=10:1, at 254 nm) to give Compound 29 (15.8 mg, 100%). LCMS: [M+H]⁺: 827.3.

The following compounds were prepared according to the above described methods using different starting materials.

| Compound | | MS(ESI): m/z |
|---|---|---|
| No. | IUPAC Name | (M + H⁺) |
| 31 | 2-((1H-pyrrolo[2,3-b]pyridin-5-yl)oxy)-4-(4-(1- | 829.3 |
| | chloro-6,7,8,9-tetrahydro-5H-benzo[7]annulen- | |
| | 5-yl)piperazin-1-yl)-N-((4-((2- | |
| | morpholinoethyl)amino)-3- | |
| | nitrophenyl)sulfonyl)benzamide | |
| 32 | 2-((1H-pyrrolo[2,3-b]pyridin-5-yl)oxy)-4-(4-(1- | 824.3 |
| | chloro-6,7,8,9-tetrahydro-5H-benzo[7]annulen- | |
| | 5-yl)piperazin-1-yl)-N-((4-((2-(4- | |
| | methylpiperazin-1-yl)ethyl)amino)-3- | |
| | nitrophenyl)sulfonyl)benzamide | |

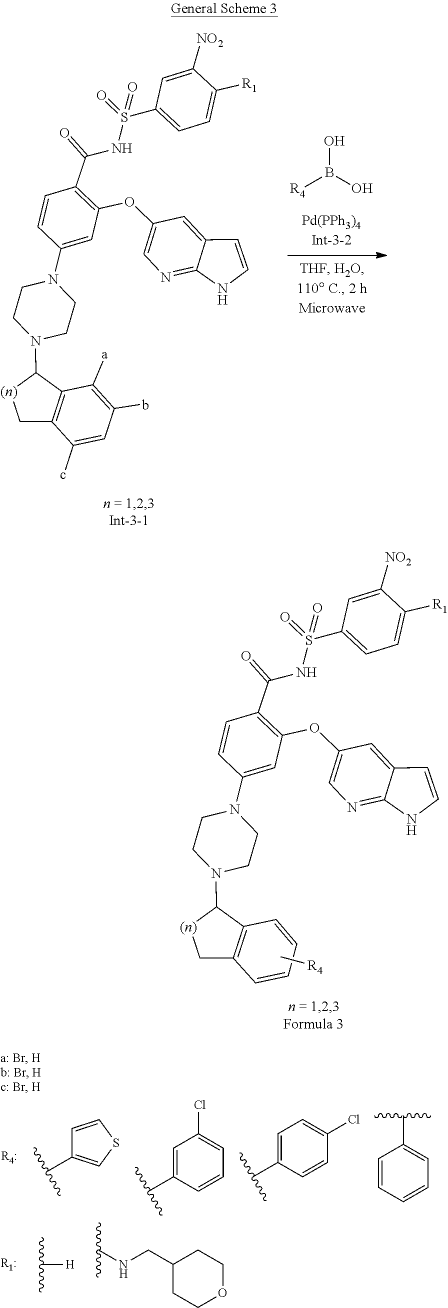

### General Step

A reaction mixture of Int-3-1 (1 eq), Int-3-2 (3 eq), K₂CO₃ (5 eq), Pd (PPh₃)₄ (0.1 eq), water (1 mL) in THE (3 mL) were heated at 80° C. for 2 h under microwave. LC-MS showed the reaction was completed. The reaction mixture was poured into DCM (50 mL) and washed with H₂O (2×25 mL). The extract was washed with brine (1×30 mL), dried over Na₂SO₄, concentrated under reduced pressure, which was further purified by prep-HPLC (base method) to give Formula 3.

### Example 4: Synthesis of Compound 11

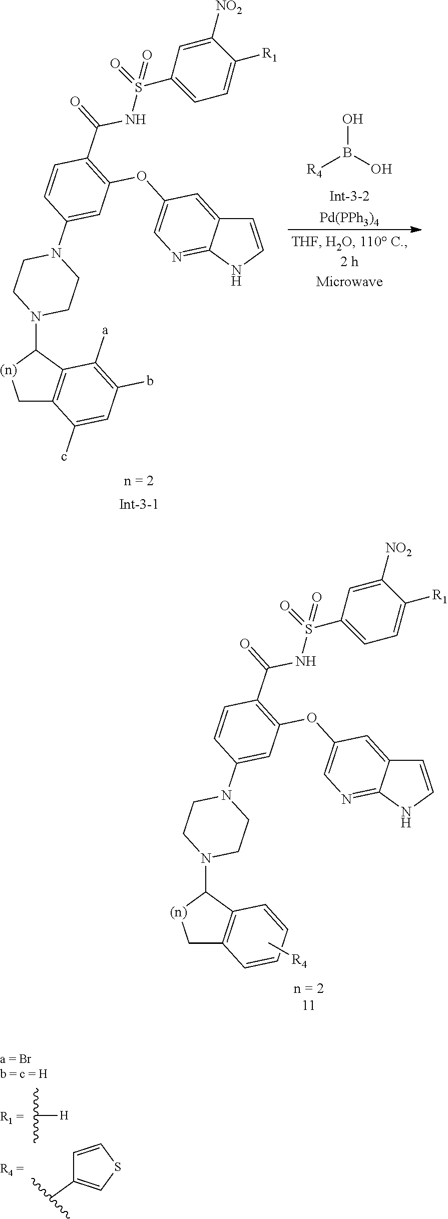

A reaction mixture of Int-3-1 (110 mg, 0.15 mmol), Int-3-2 (58 mg, 0.45 mmol), K₂CO₃ (103 mg, 0.75 mmol), Pd (PPh₃)₄ (17 mg, 0.01 mmol), water (1 mL) in THF (3 ml) were heated at 80° C. for 2 h. LC-MS showed the reaction was completed. The reaction mixture was poured into DCM (50 mL) and washed with H₂O (2×25 mL). The extract was washed with brine (1×30 mL), dried over Na₂SO₄, concentrated under reduced pressure, which was further purified by prep-HPLC (base method) to give Compound 11 (30 mg, 31%). LCMS: [M+H]⁺: 735.2.

### Example 5: Synthesis of Compound 19

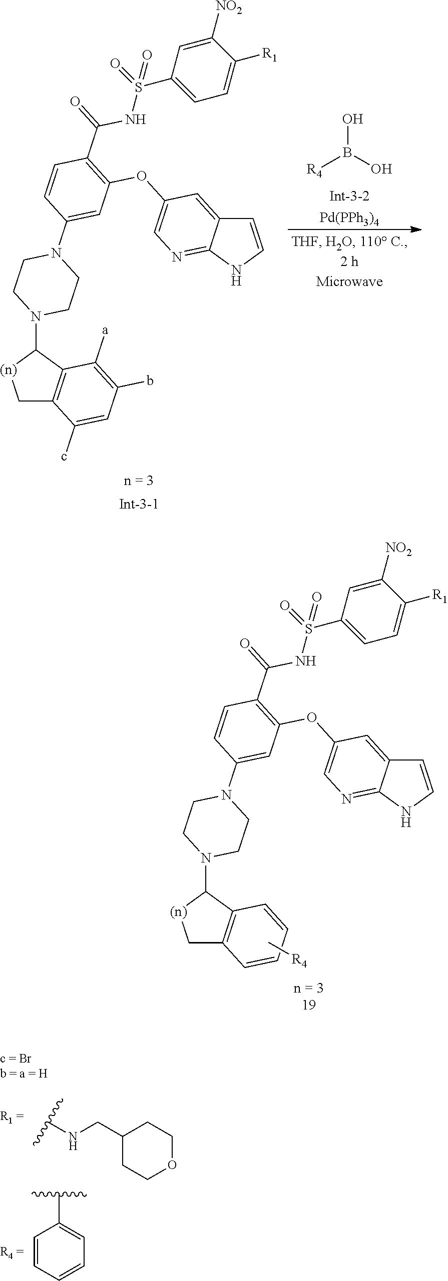

A reaction mixture of Int-3-1 (100 mg, 0.11 mmol), Int-3-2 (42 mg, 0.35 mmol), Pd(PPh₃)₄ (12 mg, 0.01 mmol), K₂CO₃ (76 mg, 0.55 mmol) in H₂O (1 ml) and THF (4 mL) was stirred for 2 h at 90° C. under microwave. LCMS showed the reaction was completed. The reaction mixture was poured into DCM (50 mL) and washed with H₂O (2×25 mL). The extract was washed with brine (1×30 mL), dried over Na₂SO₄, concentrated under reduced pressure, which was further purified by prep-HPLC (base method) to give Compound 19 (31 mg, 32%). LCMS: [M+H]⁺: 856.3.

The following compounds were prepared according to the above described methods using different starting materials.

| Compound | | MS(ESI): m/z |
|---|---|---|
| No. | IUPAC Name | (M + H⁺) |
| 13 | 2-((1H-pyrrolo[2,3-b]pyridin-5-yl)oxy)-4-(4- | 763.0 |
| | (8-(4-chlorophenyl)-1,2,3,4- | |
| | tetrahydronaphthalen-1-yl)piperazin-1-yl)-N- | |
| | ((3-nitrophenyl)sulfonyl)benzamide | |
| 14 | 2-((1H-pyrrolo[2,3-b]pyridin-5-yl)oxy)-4-(4- | 763.2 |
| | (8-(3-chlorophenyl)-1,2,3,4- | |
| | tetrahydronaphthalen-1-yl)piperazin-1-yl)-N- | |
| | ((3-nitrophenyl)sulfonyl)benzamide | |
| 15 | 2-((1H-pyrrolo[2,3-b]pyridin-5-yl)oxy)-N-((3- | 735.2 |
| | nitrophenyl)sulfonyl)-4-(4-(8-(thiophen-2-yl)- | |
| | 1,2,3,4-tetrahydronaphthalen-1-yl)piperazin-1- | |
| | yl)benzamide | |
| 16 | 2-((1H-pyrrolo[2,3-b]pyridin-5-yl)oxy)-4-(4- | 876.3 |
| | (8-(4-chlorophenyl)-1,2,3,4- | |
| | tetrahydronaphthalen-1-yl)piperazin-1-yl)-N- | |
| | ((3-nitro-4-(((tetrahydro-2H-pyran-4- | |
| | yl)methyl)amino)phenyl)sulfonyl)benzamide | |
| 7 | 2-((1H-pyrrolo[2,3-b]pyridin-5-yl)oxy)-N-((3- | 715.2 |
| | nitrophenyl)sulfonyl)-4-(4-(7-phenyl-2,3- | |
| | dihydro-1H-inden-1-yl)piperazin-1- | |
| | yl)benzamide | |
| 8 | 2-((1H-pyrrolo[2,3-b]pyridin-5-yl)oxy)-N-((3- | 729.2 |
| | nitrophenyl)sulfonyl)-4-(4-(8-phenyl-1,2,3,4- | |
| | tetrahydronaphthalen-1-yl)piperazin-1- | |
| | yl)benzamide | |
| 22 | 2-((1H-pyrrolo[2,3-b]pyridin-5-yl)oxy)-N-((3- | 856.2 |
| | nitro-4-(((tetrahydro-2H-pyran-4- | |
| | yl)methyl)amino)phenyl)sulfonyl)-4-(4-(3- | |
| | phenyl-6,7,8,9-tetrahydro-5H- | |
| | benzo[7]annulen-5-yl)piperazin-1- | |
| | yl)benzamide | |

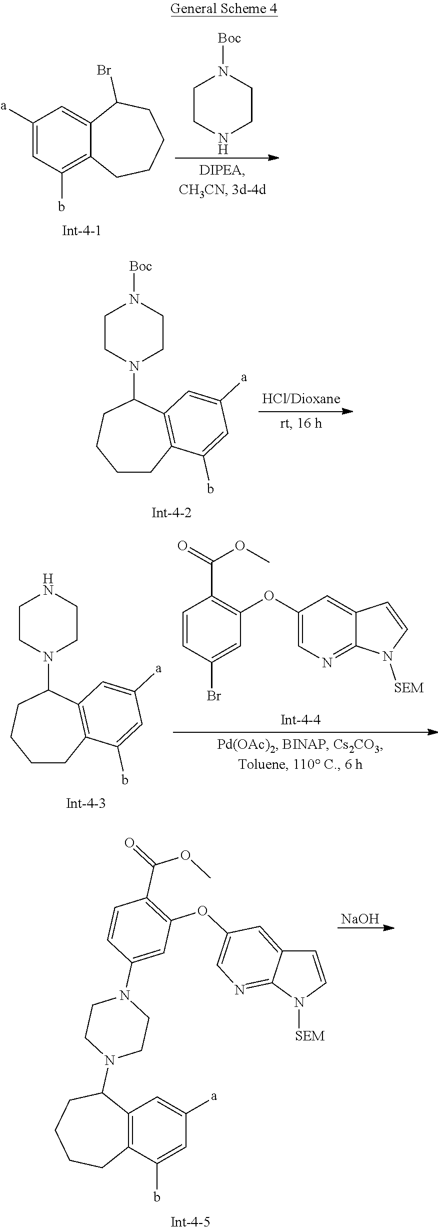

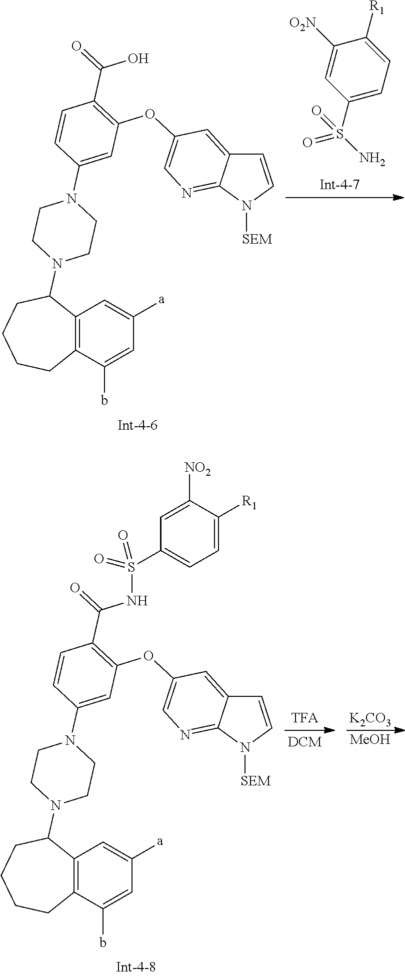

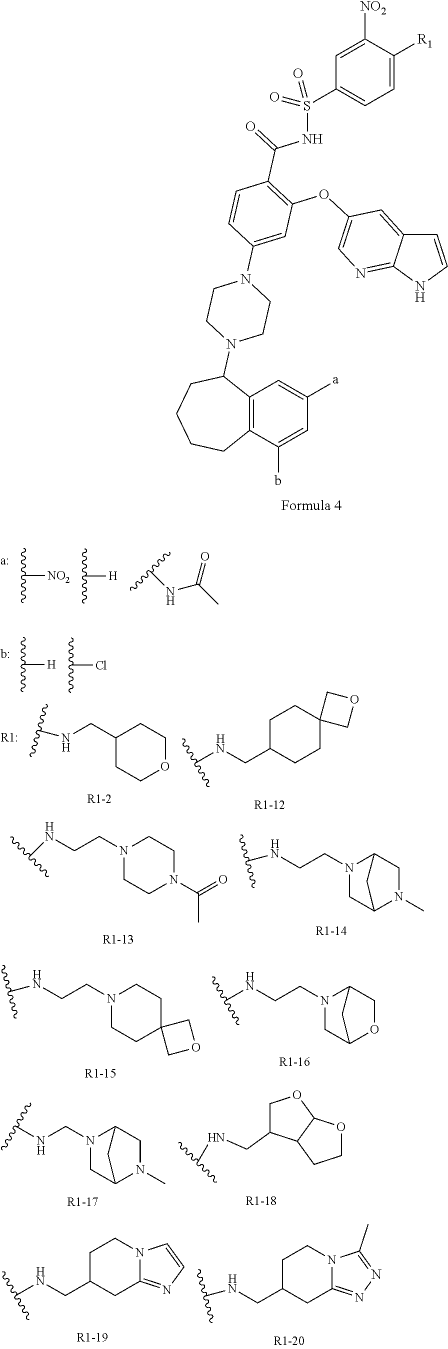

### General Step 1: Synthesis of Int-4-2

A mixture of Int-4-1 (1 eq) and tert-butyl piperazine-1-carboxylate (2 eq) in MeCN (80 mL) was stirred at 35° C. for 72 h. LC-MS showed the reaction was completed. MeCN was removed under reduced pressure to give crude product. The crude product was purified by combine flash (PE:DCM=100:0 to 50:50 to 0:100) (in 214 nm) to give Int-4-2.

### General Step 2: Synthesis of Int-4-3

To a solution of Int-4-2 (1 eq) in anhydrous DCM (20 mL) was added HCl/Dioxane (4M) at rt. The reaction mixture was stirred at rt for 16 h. LC-MS showed the reaction was completed. DCM and dioxane were removed under reduced pressure to give crude Int-4-3. The crude product was used for next step without purification.

### General Step 3: Synthesis of Int-4-5

To a solution of Int-4-3 (1 eq) and Int-4-4 (1.2 eq) in anhydrous toluene (20 mL) was added Pd(OAc)₂ (0.1 eq), BINAP (0.1 eq), and Cs₂CO₃ (3 eq) at rt. The reaction mixture was stirred at 110° C. for 6 h under N₂ atmosphere. LCMS showed the reaction was completed. Toluene was removed under reduced pressure. The crude product was purified by combine flash (PE:EtOAc=100:0 to 90:10 to 80:20, at 254 nm) to give Int-4-5.

### General Step 4: Synthesis of Int-4-6

To a solution of Int-4-5 (1 eq) in MeOH (10 mL) was added NaOH (10 eq) solution (H₂O, 5 mL). The reaction mixture was stirred at 60° C. for 6 h. LC-MS showed the reaction was completed. The residue was treated with aqueous HCl (1N, 5 mL) to pH=5. The reaction mixture was poured into water (10 mL) and extracted with EtOAc (3×25 mL). The combined extracts were washed with brine (2×40 mL), dried over Na₂SO₄, concentrated to afford Int-4-6.

### General Step 5: Synthesis of Int-4-8

To a solution of Int-4-6 (1 eq) and Int-4-7 (1.25 eq) in anhydrous DMF (10 mL) and DCM (10 mL) was added EDCI (1.2 eq), DMAP (4 eq), and DIPEA (3 eq) at rt. The reaction mixture was stirred at 35° C. for 16 h under N₂ atmosphere. LC-MS showed the reaction was completed. The reaction mixture was poured into EtOAc (30 mL) and washed with H₂O (2×25 mL). The extract was washed with brine (1×20 mL), dried over Na₂SO₄, concentrated. The crude product was purified by combine flash (PE:EtOAc=100:0 to DCM:MeOH=100 to 90:10 to 80:20, at 254 nm) to give Int-4-8.

### General Step 6: Synthesis of Formula 4

To a solution of Int-4-8 (1 eq) in anhydrous DCM (3 mL) was added TFA (1 mL) at rt. The reaction mixture was stirred at rt for 2 h under N₂ atmosphere. LC-MS showed the reaction was completed. The reaction mixture was concentrated. MeOH (3 mL) was added K₂CO₃ in H₂O (1 mL) at rt. The reaction mixture was stirred at rt for 16 h under N₂ atmosphere. LC-MS showed the reaction was completed. The MeOH was removed under reduced pressure, 50 ml DCM was added to the mixture, dried over Na₂SO₄, concentrated to afford crude product. The crude product was purified by TLC (DCM:MeOH=10:1, at 254 nm) to give Formula 4.

### Example 6: Synthesis of Compound 64

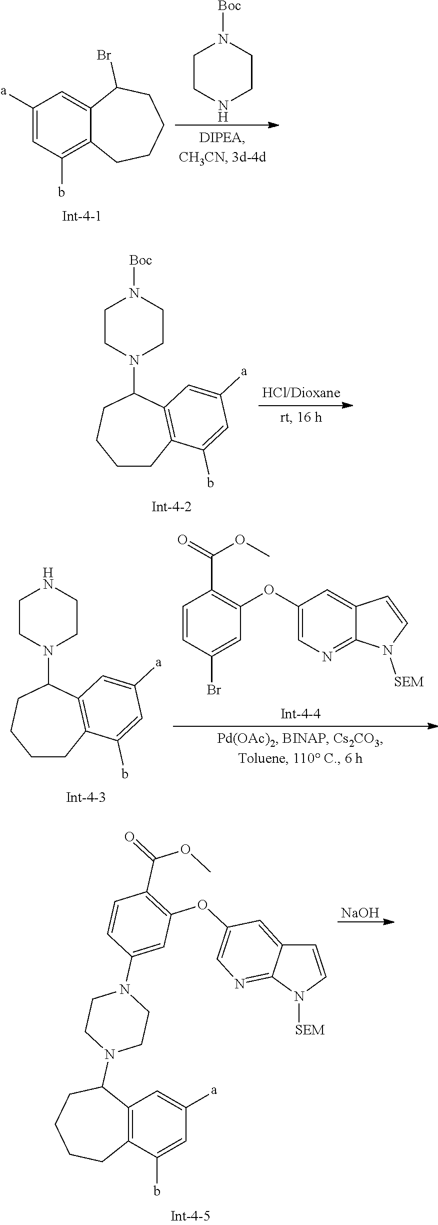

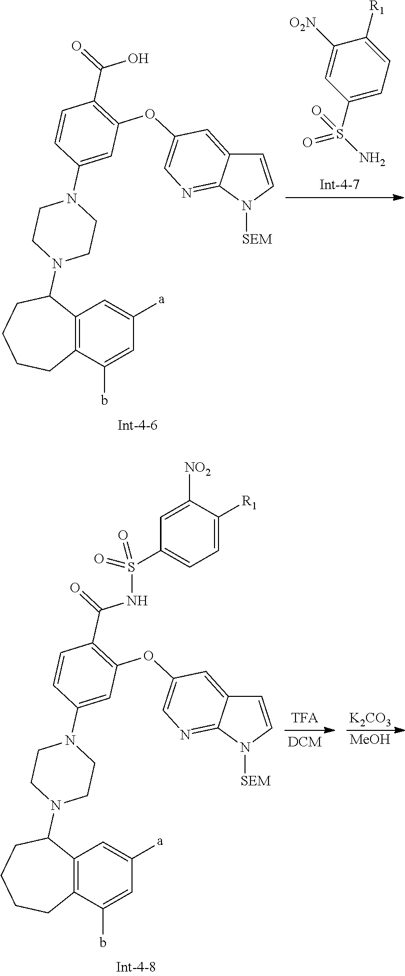

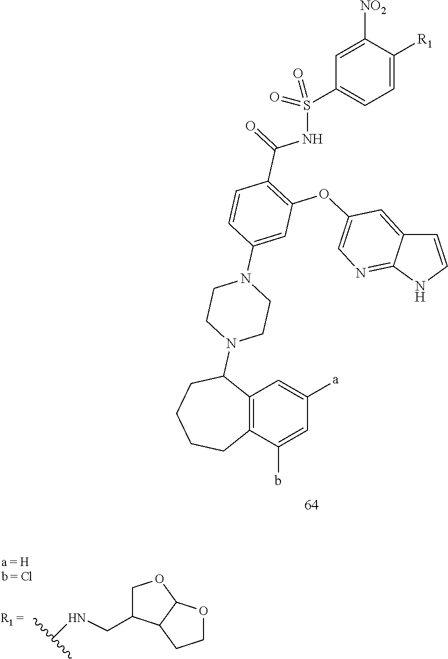

### Step 1: Synthesis of Int-4-2

A mixture of Int-4-1 (1.6 g, 6.2 mmol) and tert-butyl piperazine-1-carboxylate (3.4 g, 18.6 mmol) in MeCN (80 mL) was stirred at 35° C. for 72 h. LC-MS showed the reaction was completed. MeCN was removed under reduced pressure to give crude product. The crude product was purified by combine flash (PE:DCM=100:0 to 50:50 to 0:100) (in 214 nm) to give Int-4-2 (1.38 g). LCMS: [M+H]⁺: 365.3.

### Step 2: Synthesis of Int-4-3

To a solution of Int-4-2 (1.38 g, 3.8 mmol) in anhydrous DCM (20 mL) was added HCl/Dioxane (4M, 8 mL) at rt. The reaction mixture was stirred at rt for 16 h. LC-MS showed the reaction was completed. DCM and dioxane were removed under reduced pressure to give crude Int-4-3 (1.4 g). The crude product was used for next step without purification. LCMS: [M+H]⁺: 265.2

### Step 3: Synthesis of Int-4-5

To a solution of Int-4-3 (300 mg, 1.2 mmol) and Int-4-4 (670 mg, 1.4 mmol) in anhydrous toluene (20 mL) was added Pd(OAc)₂ (30 mg, 0.12 mmol), BINAP (75 mg, 0.12 mmol), and Cs₂CO₃ (1.75 g, 4.8 mmol) at rt. The reaction mixture was stirred at 110° C. for 6 h under N₂ atmosphere. LC-MS showed the reaction was completed. Toluene was removed under reduced pressure to give 500 mg of crude product. The crude product was purified by combine flash (PE:EtOAc=100:0 to 90:10 to 80:20, at 254 nm) to give Int-4-5 (420 mg). LCMS: [M+H]⁺: 661.3

### Step 4: Synthesis of Int-4-6

To a solution of Int-4-5 (420 mg, 0.63 mmol) in MeOH (20 mL) was added NaOH (252 mg, 6.3 mmol) solution (H₂O, 6 mL). The reaction mixture was stirred at 60° C. for 6 hours. LC-MS showed the reaction was completed. The residue was treated with aqueous HCl (1N, 5 mL) to pH=5. The reaction mixture was poured into water (10 mL) and extracted with EtOAc (3×25 mL). The combined extracts were washed with brine (2×40 mL), dried over Na₂SO₄, concentrated to afford Int-4-6 (380 mg, 93%). LCMS: [M+H]⁺: 647.3

### Step 5: Synthesis of Int-4-8

To a solution of Int-4-6 (65 mg, 0.1 mmol) and Int-4-7 (41 mg, 0.12 mmol) in anhydrous DMF (1 mL) and DCM (1 mL) was added EDCI (25 mg, 0.13 mmol), DMAP (48 mg, 0.4 mmol), and DIPEA (38 mg, 0.3 mmol) at rt. The reaction mixture was stirred at 35° C. for 16 h under N₂ atmosphere. LC-MS showed the reaction was completed. The reaction mixture was poured into EtOAc (30 mL) and washed with H₂O (2×25 mL). The extract was washed with brine (1×20 mL), dried over Na₂SO₄, concentrated. The crude product was purified by combine flash (PE:EtOAc=100:0 to DCM:MeOH=100 to 90:10 to 80:20, at 254 nm) to give Int-4-8 (80%). LCMS: [M+H]⁺=972.3.

### Step 6: Synthesis of Compound 64

To a solution of Int-4-8 (50 mg, 0.2 mmol) in anhydrous DCM (5 mL) was added TFA (1 mL) at rt. The reaction mixture was stirred at rt for 2 h under N₂ atmosphere. LC-MS showed the reaction was completed. The reaction mixture was concentrated. MeOH (3 mL) was added K₂CO₃ in H₂O (1 mL) at rt. The reaction mixture was stirred at rt for 16 h under N₂ atmosphere. LC-MS showed the reaction was completed. The MeOH was removed under reduced pressure, 50 ml DCM was added to the mixture, dried over Na₂SO₄, concentrated to afford crude product. The crude product was purified by TLC (DCM:MeOH=10:1, at 254 nm) to give Compound 64 (10 mg, 20%). LCMS: [M+H]⁺=841.2.

### Example 7: Synthesis of Compound 42

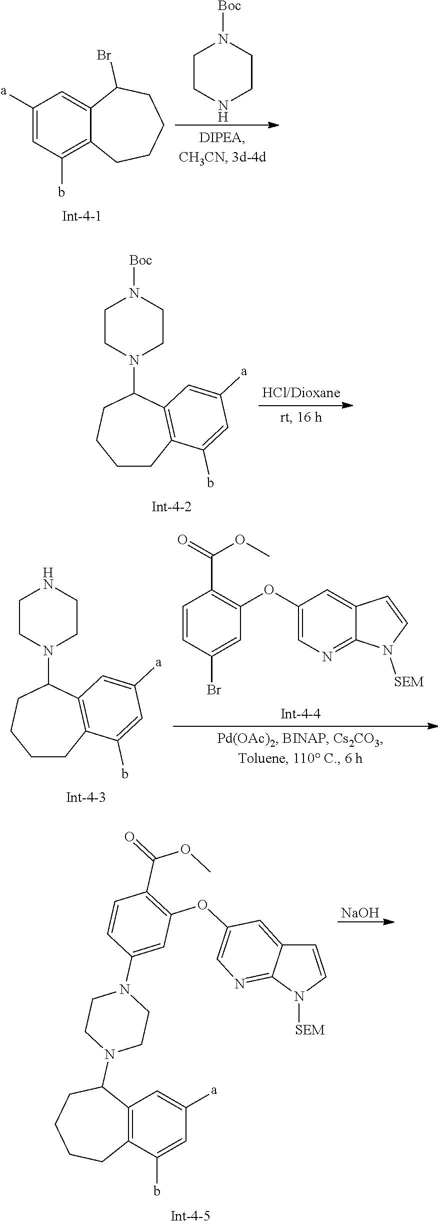

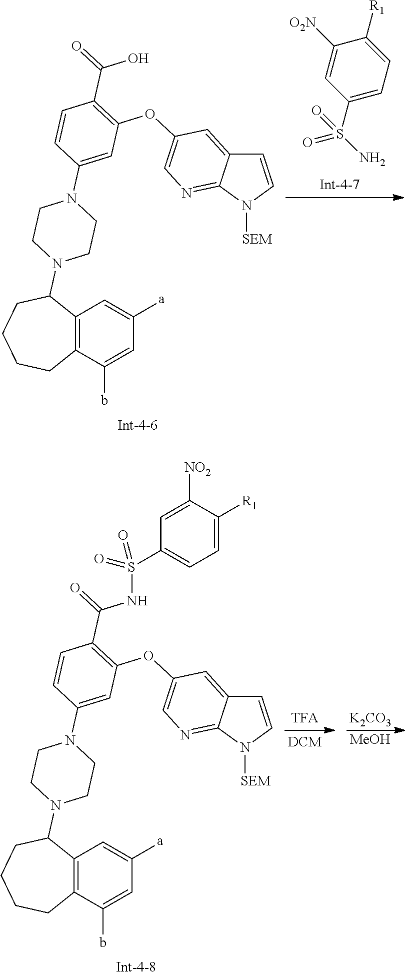

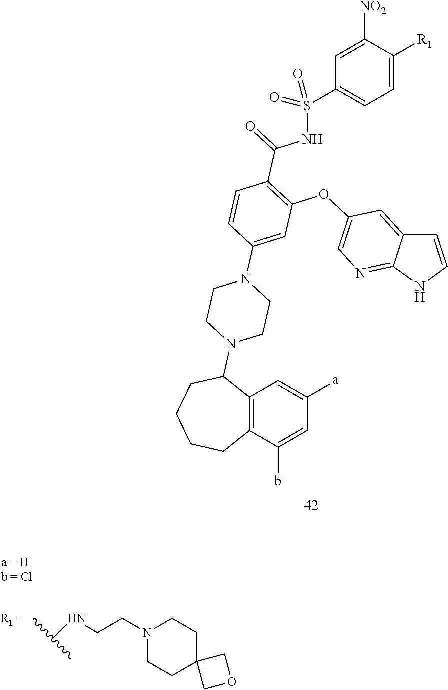

### Step 1: Synthesis of Int-4-8

To a solution of Int-4-6 (73 mg, 0.14 mmol) and Int-4-7 (40 mg, 0.12 mmol) in anhydrous DMF (1 mL) and DCM (1 mL) was added EDCI (40 mg, 0.21 mmol), DMAP (68 mg, 0.56 mmol), and DIPEA (55 mg, 0.42 mmol) at rt. The reaction mixture was stirred at 35° C. for 16 h under N₂ atmosphere. LC-MS showed the reaction was completed. The reaction mixture was poured into EtOAc (30 mL) and washed with H₂O (2×25 mL). The extract was washed with brine (1×20 mL), dried over Na₂SO₄, concentrated. The crude product was purified by combine flash (PE:EtOAc=100:0 to DCM:MeOH=100 to 90:10 to 80:20, at 254 nm) to give Int-4-8 (80 mg, 74%). LCMS: [M+H]⁺=999.3.

### Step 2: Synthesis of Compound 42

To a solution of Int-4-8 (80 mg, 0.08 mmol) in anhydrous DCM (5 mL) was added TFA (1 mL) at rt. The reaction mixture was stirred at rt for 2 h under N₂ atmosphere. LC-MS showed the reaction was completed. The reaction mixture was concentrated. MeOH (3 mL) was added K₂CO₃ in H₂O (1 mL) at rt. The reaction mixture was stirred at rt for 16 h under N₂ atmosphere. LC-MS showed the reaction was completed. The MeOH was removed under reduced pressure, 50 ml DCM was added to the mixture, dried over Na₂SO₄, concentrated to afford crude product. The crude product was purified by TLC (DCM:MeOH=10:1, at 254 nm) to give Compound 42 (38.8 mg, 56%). LCMS: [M+H]⁺=869.2.

The following compounds were prepared according to the above described methods using different starting materials.

| Compound | | MS(ESI): m/z |
|---|---|---|
| No. | IUPAC Name | (M + H⁺) |
| 30 | 2-((1H-pyrrolo[2,3-b]pyridin-5-yl)oxy)-N-((3- | 825.2 |
| | nitro-4-(((tetrahydro-2H-pyran-4- | |
| | yl)methyl)amino)phenyl)sulfonyl)-4-(4-(3-nitro- | |
| | 6,7,8,9-tetrahydro-5H-benzo[7]annulen-5- | |
| | yl)piperazin-1-yl)benzamide | |
| 34 | 2-((1H-pyrrolo[2,3-b]pyridin-5-yl)oxy)-4-(4-(3- | 837.3 |
| | acetamido-6,7,8,9-tetrahydro-5H-benzo[7]annulen- | |
| | 5-yl)piperazin-1-yl)-N-((3-nitro-4-(((tetrahydro- | |
| | 2H-pyran-4- | |
| | yl)methyl)amino)phenyl)sulfonyl)benzamide | |
| 33 | 2-((1H-pyrrolo[2,3-b]pyridin-5-yl)oxy)-N-((3- | 825.2 |
| | nitro-4-(((tetrahydro-2H-pyran-4- | |
| | yl)methyl)amino)phenyl)sulfonyl)-4-(4-(1-nitro- | |
| | 6,7,8,9-tetrahydro-5H-benzo[7]annulen-5- | |
| | yl)piperazin-1-yl)benzamide | |
| 38 | 2-((1H-pyrrolo[2,3-b]pyridin-5-yl)oxy)-N-((4-(((2- | 854.3 |
| | oxaspiro[3.5]nonan-7-yl)methyl)amino)-3- | |
| | nitrophenyl)sulfonyl)-4-(4-(1-chloro-6,7,8,9- | |
| | tetrahydro-5H-benzo[7]annulen-5-yl)piperazin-1- | |
| | yl)benzamide | |
| 39 | 2-((1H-pyrrolo[2,3-b]pyridin-5-yl)oxy)-N-((4-((2- | 870.2 |
| | (4-acetylpiperazin-1-yl)ethyl)amino)-3- | |
| | nitrophenyl)sulfonyl)-4-(4-(1-chloro-6,7,8,9- | |
| | tetrahydro-5H-benzo[7]annulen-5-yl)piperazin-1- | |
| | yl)benzamide | |
| 41 | 2-((1H-pyrrolo[2,3-b]pyridin-5-yl)oxy)-N-((4-((2- | 841.3 |
| | (2-oxa-5-azabicyclo[2.2.1]heptan-5- | |
| | yl)ethyl)amino)-3-nitrophenyl)sulfonyl)-4-(4-(1- | |
| | chloro-6,7,8,9-tetrahydro-5H-benzo[7]annulen-5- | |
| | yl)piperazin-1-yl)benzamide | |
| 43 | 2-((1H-pyrrolo[2,3-b]pyridin-5-yl)oxy)-4-(4-(1- | 854.3 |
| | chloro-6,7,8,9-tetrahydro-5H-benzo[7]annulen-5- | |
| | yl)piperazin-1-yl)-N-((4-((2-(5-methyl-2,5- | |
| | diazabicyclo[2.2.1]heptan-2-yl)ethyl)amino)-3- | |
| | nitrophenyl)sulfonyl)benzamide | |
| 44 | 2-((1H-pyrrolo[2,3-b]pyridin-5-yl)oxy)-4-(4-(1- | 839.3 |
| | chloro-6,7,8,9-tetrahydro-5H-benzo[7]annulen-5- | |
| | yl)piperazin-1-yl)-N-((4-(((2-methyl-2- | |
| | azabicyclo[2.2.1 ]heptan-5-yl)methyl)amino)-3- | |
| | nitrophenyl)sulfonyl)benzamide | |
| 46 | 2-((1H-pyrrolo[2,3-b]pyridin-5-yl)oxy)-N-((4-(((2- | 826.3 |
| | oxaspiro[3.3]heptan-6-yl)methyl)amino)-3- | |
| | nitrophenyl)sulfonyl)-4-(4-(1-chloro-6,7,8,9- | |
| | tetrahydro-5H-benzo[7]annulen-5-yl)piperazin-1- | |
| | yl)benzamide | |
| 53 | 2-((1H-pyrrolo[2,3-b]pyridin-5-yl)oxy)-4-(4-(1- | |
| | chloro-6,7,8,9-tetrahydro-5H-benzo[7]annulen-5- | |
| | yl)piperazin-1-yl)-N-((3-nitro-4-(((5,6,7,8- | 850.3 |
| | tetrahydroimidazo[1,2-a]pyridin-7- | |
| | yl)methyl)amino)phenyl)sulfonyl)benzamide | |
| 57 | 2-((1H-pyrrolo[2,3-b]pyridin-5-yl)oxy)-4-(4-(1- | 865.3 |
| | chloro-6,7,8,9-tetrahydro-5H-benzo[7]annulen-5- | |
| | yl)piperazin-1-yl)-N-((4-(((3-methyl-5,6,7,8- | |
| | tetrahydro-[1,2,4]triazolo[4,3-a]pyridin-6- | |
| | yl)methyl)amino)-3- | |
| | nitrophenyl)sulfonyl)benzamide | |

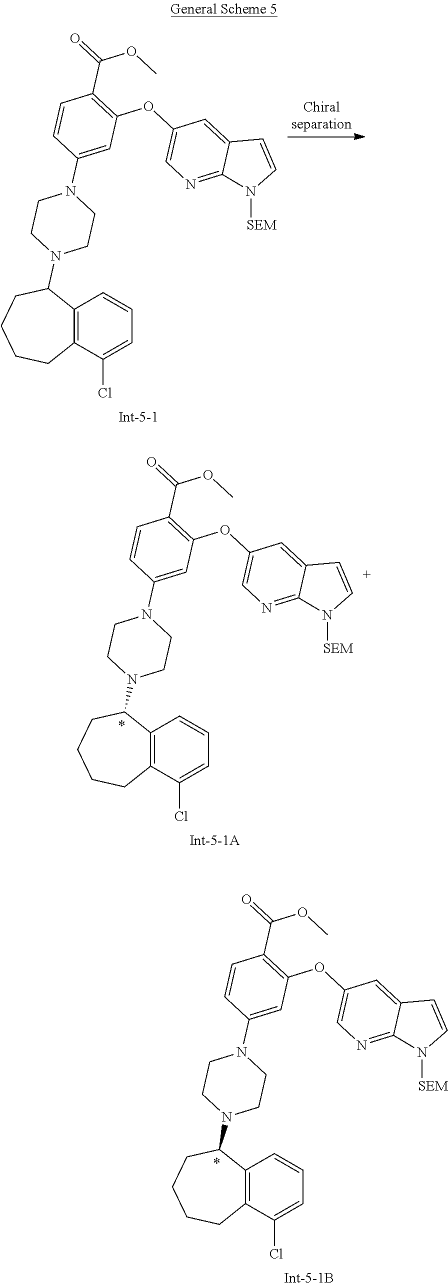 [SMILES: COC(=O)c1ccc(N2CCN(C3CCCCc4c(Cl)cccc43)CC2)cc1Oc1cnc2c(ccn2COCC[Si](C)(C)C)c1 ; COC(=O)c1ccc(N2CCN([C@H]3CCCCc4c(Cl)cccc43)CC2)cc1Oc1cnc2c(ccn2COCC[Si](C)(C)C)c1 ; COC(=O)c1ccc(N2CCN([C@@H]3CCCCc4c(Cl)cccc43)CC2)cc1Oc1cnc2c(ccn2COCC[Si](C)(C)C)c1]

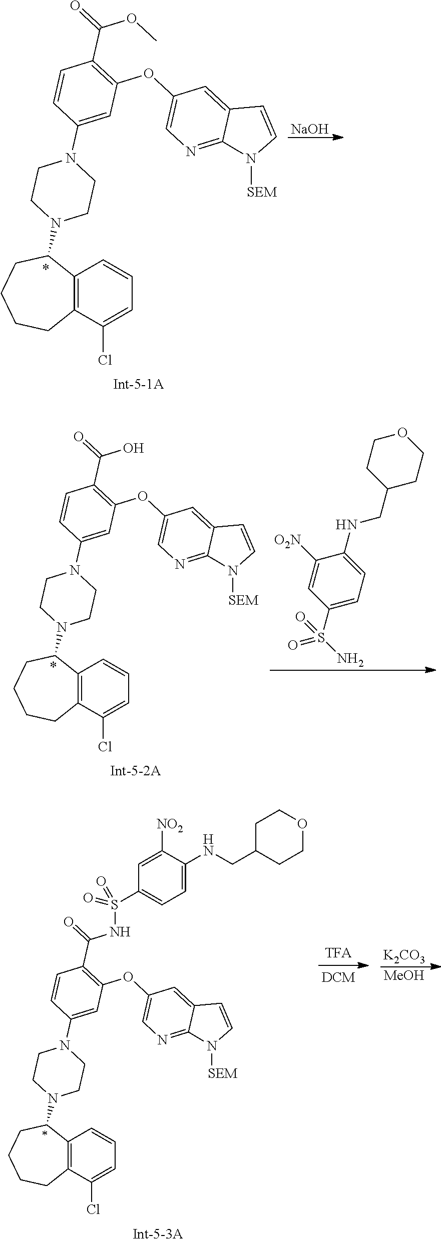

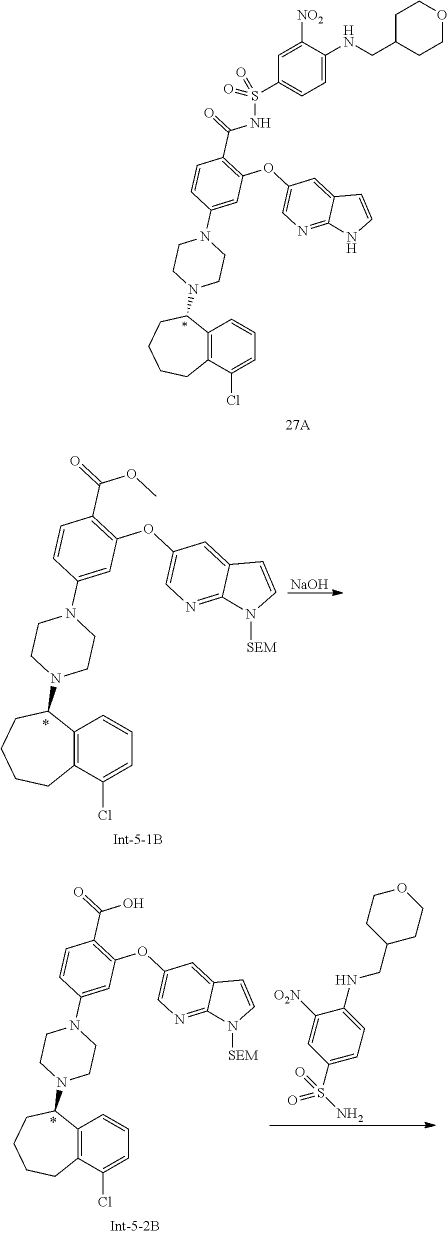

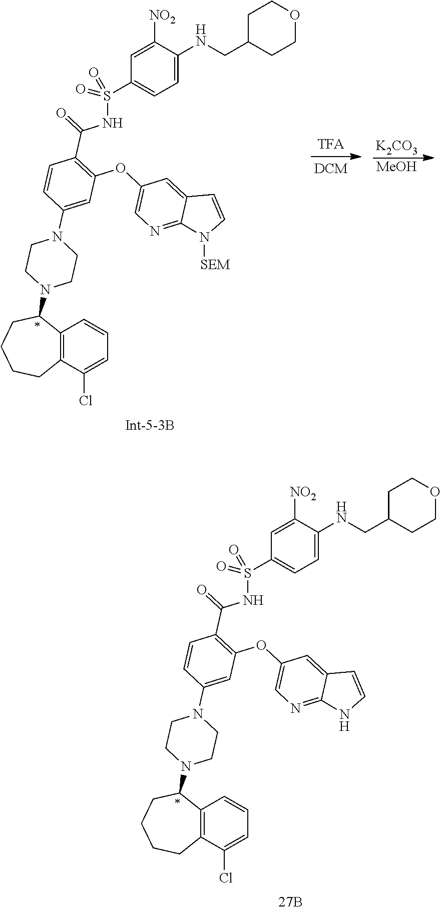 [SMILES: "MeOH" ; C[Si](C)(C)CCOCn1ccc2cc(Oc3cc(N4CCN([C@@H]5CCCCc6c(Cl)cccc65)CC4)ccc3C(=O)NS(=O)(=O)c3ccc(NCC4CCOCC4)c([N+](=O)[O-])c3)cnc21 ; "K2CO3" ; O=C(NS(=O)(=O)c1ccc(NCC2CCOCC2)c([N+](=O)[O-])c1)c1ccc(N2CCN([C@@H]3CCCCc4c(Cl)cccc43)CC2)cc1Oc1cnc2[nH]ccc2c1]

### Example 8: Synthesis of Compound 27A

### Step 1: Synthesis of Int-5-1A and Int-5-1B

Int-5-1 was sent to chiral separation to give Int-5-1A and Int-5-1B.

### Step 2: Synthesis of Int-5-2A

To a solution of Int-5-1A (150 mg, 0.23 mmol) in MeOH (10 mL) was added NaOH (0.1 g, 2.3 mmol) solution (H₂O, 2 mL). The reaction mixture was stirred at 60° C. for 6 hours. LC-MS showed the reaction was completed. The residue was treated with aqueous HCl (1N, 5 mL) to pH=5. The reaction mixture was poured into water (10 mL) and extracted with EtOAc (3×25 mL). The combined extracts were washed with brine (2×40 mL), dried over Na₂SO₄, concentrated to afford Int-5-2A (135 mg). LCMS: [M+H]⁺: 647.3.

### Step 3: Synthesis of Int-5-3A

To a solution of Int-5-2A (100 mg, 0.15 mmol) and 3-nitro-4-(((tetrahydro-2H-pyran-4-yl) methyl) amino) benzenesulfonamide (63 mg, 0.20 mmol) in anhydrous DMF (3 mL) and DCM (3 mL) was added EDCI (40 mg, 0.20 mmol), DMAP (55 mg, 0.45 mmol), and DIPEA (58 mg, 0.45 mmol) at rt. The reaction mixture was stirred at 35° C. for 16 h under N₂ atmosphere. LC-MS showed the reaction was completed. The reaction mixture was poured into EtOAc (30 mL) and washed with H₂O (2×25 mL). The extract was washed with brine (1×20 mL), dried over Na₂SO₄, concentrated to afford 150 mg of crude product. The crude product was purified by TLC (DCM:MeOH=95:5, at 254 nm) to give Int-5-3A (100 mg). LCMS: [M+H]⁺: 944.1.

### Step 4: Synthesis of Compound 27A

To a solution of Int-5-3A (100 mg, 0.11 mmol) in anhydrous DCM (5 mL) was added TFA (1.5 mL) at rt. The reaction mixture was stirred at rt for 2 h under N₂ atmosphere. LC-MS showed the reaction was completed. DCM was removed and MeOH (5 mL) was added K₂CO₃ in H₂O (1 mL) at rt. The reaction mixture was stirred at rt for 16 h under N₂ atmosphere. LC-MS showed the reaction was completed. The MeOH was removed under reduced pressure, 50 ml DCM was added to the mixture, dried over Na₂SO₄, concentrated to afford crude product. The crude product was purified by TLC (DCM:MeOH=10:1, at 254 nm) to give Compound 27A (34.9 mg, 100%). LCMS: [M+H]⁺: 814.1.

### Example 9: Synthesis of Compound 27B

### Step 1: Synthesis of Int-5-2B

To a solution of Int-5-1B (130 mg, 0.19 mmol) in MeOH (5 mL) was added NaOH (0.08 g, 2.0 mmol) solution (H₂O, 1 mL). The reaction mixture was stirred at 60° C. for 6 hours. LC-MS showed the reaction was completed. The residue was treated with aqueous HCl (1N, 5 mL) to pH=5. The reaction mixture was poured into water (10 mL) and extracted with EtOAc (3×25 mL). The combined extracts were washed with brine (2×40 mL), dried over Na₂SO₄, concentrated to afford Int-5-2B (120 mg). LCMS: [M+H]⁺: 647.1

### Step 2: Synthesis of Int-5-3B

To a solution of Int-5-2B (120 mg, 0.19 mmol) and 3-nitro-4-(((tetrahydro-2H-pyran-4-yl) methyl) amino) benzenesulfonamide (83 mg, 0.25 mmol) in anhydrous DMF (3 mL) and DCM (3 mL) was added EDCI (55 mg, 0.23 mmol), DMAP (70 mg, 0.57 mmol), and DIPEA (90 mg, 0.57 mmol) at rt. The reaction mixture was stirred at 35° C. for 16 h under N₂ atmosphere. LC-MS showed the reaction was completed. The reaction mixture was poured into EtOAc (30 mL) and washed with H₂O (2×25 mL). The extract was washed with brine (1×20 mL), dried over Na₂SO₄, concentrated to afford 150 mg of crude product. The crude product was purified by TLC (DCM:MeOH=95:5, at 254 nm) to give Int-5-3B (100 mg). LCMS: [M+H]⁺: 944.3.

### Step 3: Synthesis of Compound 27B

To a solution of Int-5-3B (100 mg, 0.11 mmol) in anhydrous DCM (5 mL) was added TFA (1.5 mL) at rt. The reaction mixture was stirred at rt for 2 h under N₂ atmosphere. LC-MS showed the reaction was completed DCM was removed and MeOH (5 mL) was added K₂CO₃ in H₂O (1 mL) at rt. The reaction mixture was stirred at rt for 16 h under N₂ atmosphere. LC-MS showed the reaction was completed. The MeOH was removed under reduced pressure, 50 ml DCM was added to the mixture, dried over Na₂SO₄, concentrated to afford crude product. The crude product was purified by TLC (DCM:MeOH=10:1, at 254 nm) to give Compound 27B (38.8 mg, 100%). LCMS: [M+H]⁺: 814.1.

The names and MS (ESI) data of Compound 27A and 27B are shown as follows.

| Compound | | MS(ESI): m/z |
|---|---|---|
| No. | IUPAC Name | (M + H⁺) |
| 27A | (S)-2-((1H-pyrrolo[2,3-b]pyridin-5-yl)oxy)-4-(4- | 814.1 |
| | (1-chloro-6,7,8,9-tetrahydro-5H- | |
| | benzo[7]annulen-5-yl)piperazin-1-yl)-N-((3- | |
| | nitro-4-(((tetrahydro-2H-pyran-4- | |
| | yl)methyl)amino)phenyl)sulfonyl)benzamide | |
| 27B | (R)-2-((1H-pyrrolo[2,3-b]pyridin-5-yl)oxy)-4-(4- | 814.2 |
| | (1-chloro-6,7,8,9-tetrahydro-5H- | |
| | benzo[7]annulen-5-yl)piperazin-1-yl)-N-((3- | |
| | nitro-4-(((tetrahydro-2H-pyran-4- | |
| | yl)methyl)amino)phenyl)sulfonyl)benzamide | |

The following compounds were prepared according to the above described methods using different starting materials.

| Compound | | MS(ESI): m/z |
|---|---|---|
| No. | IUPAC Name | (M + H⁺) |
| 1A | (R-)2-((1H-pyrrolo[2,3-b]pyridin-5-yl)oxy)-N- | 653.1 |
| | ((3-nitrophenyl)sulfonyl)-4-(4-(1,2,3,4- | |
| | tetrahydronaphthalen-1-yl)piperazin-1- | |
| | yl)benzamide | |
| 1B | (S-)-2-((1H-pyrrolo[2,3-b]pyridin-5-yl)oxy)-N- | 653.1 |
| | ((3-nitrophenyl)sulfonyl)-4-(4-(1,2,3,4- | |
| | tetrahydronaphthalen-1-yl)piperazin-1- | |
| | yl)benzamide | |
| 3A | (S)-2-((1H-pyrrolo[2,3-b]pyridin-5-yl)oxy)-4-(4- | 639.2 |
| | (2,3-dihydro-1H-inden-1-yl)piperazin-1-yl)-N- | |
| | ((3-nitrophenyl)sulfonyl)benzamide | |
| 3B | (R)-2-((1H-pyrrolo[2,3-b]pyridin-5-yl)oxy)-4-(4- | 639.2 |
| | (2,3-dihydro-1H-inden-1-yl)piperazin-1-yl)-N- | |
| | ((3-nitrophenyl)sulfonyl)benzamide | |
| 5A | (S)-2-((1H-pyrrolo[2,3-b]pyridin-5-yl)oxy)-N- | 667.3 |
| | ((3-nitrophenyl)sulfonyl)-4-(4-(6,7,8,9- | |
| | tetrahydro-5H-benzo[7]annulen-5-yl)piperazin- | |
| | 1-yl)benzamide | |
| 5B | (R)-2-((1H-pyrrolo[2,3-b]pyridin-5-yl)oxy)-N- | 667.3 |
| | ((3-nitrophenyl)sulfonyl)-4-(4-(6,7,8,9- | |
| | tetrahydro-5H-benzo[7]annulen-5-yl)piperazin- | |
| | 1-yl)benzamide | |
| 6A | (S)-2-((1H-pyrrolo[2,3-b]pyridin-5-yl)oxy)-N- | 780.2 |
| | ((3-nitro-4-(((tetrahydro-2H-pyran-4- | |
| | yl)methyl)amino)phenyl)sulfonyl)-4-(4-(6,7,8,9- | |
| | tetrahydro-5H-benzo[7]annulen-5-yl)piperazin- | |
| | 1-yl)benzamide | |
| 6B | (R)-2-((1H-pyrrolo[2,3-b]pyridin-5-yl)oxy)-N- | 780.3 |
| | ((3-nitro-4-(((tetrahydro-2H-pyran-4- | |
| | yl)methyl)amino)phenyl)sulfonyl)-4-(4-(6,7,8,9- | |
| | tetrahydro-5H-benzo[7]annulen-5-yl)piperazin- | |
| | 1-yl)benzamide | |

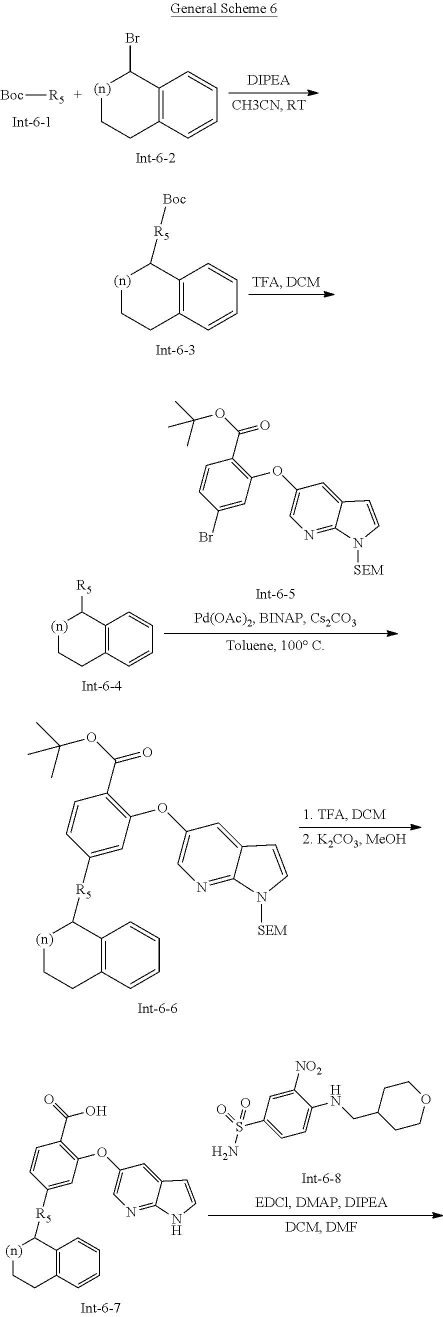

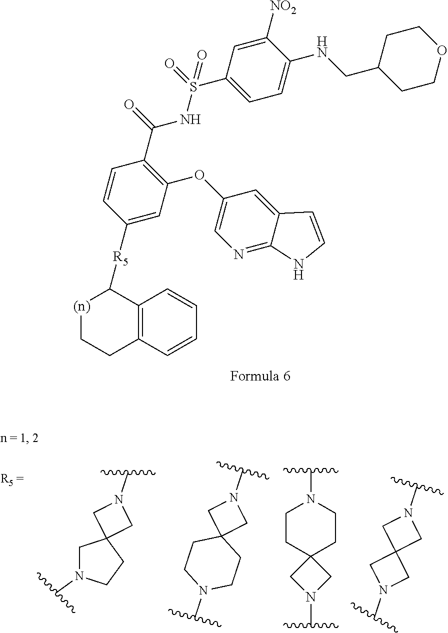

### General Step 1: Synthesis of Int-6-3

To a solution of Int-6-1 (1 eq) and Int-6-2 (1 eq) in CH₃CN (8 mL) was added DIEA (2 eq) and the mixture was stirred at rt overnight. EA (30 mL) was added the mixture was washed with water (20 mL×3), brine, dried over anhydrous Na₂SO₄ and concentrated. The crude residue was purified by combine flash (EA/PE=0˜60%) to give Int-6-3.

### General Step 2: Synthesis of Int-6-4

To a solution of Int-6-3 (1 eq) in DCM (3 mL) was added TFA (1 mL). The reaction mixture was stirred at rt for 1 h. LC-MS monitored and the starting material was consumed completely. The mixture was concentrated under vacuum to give Int-6-4. The crude product was used directly in the next step.

### General Step 3: Synthesis of Int-6-6

To a solution of Int-6-4 (1 eq), Int-6-5 (1 eq), Pd(OAc)₂ (0.2 eq), BINAP (0.3 eq) and Cs₂CO₃ (3 eq) in toluene (5 mL) was heated at 100° C. overnight. LC-MS monitored and desired product produced. H₂O (30 mL) was added and the mixture was extracted with EA (20 mL×3). The combined organic phase was washed with brine, dried over anhydrous Na₂SO₄ and concentrated. The crude residue was purified by combine flash (EA/PE=0˜60%) to give Int-6-6.

### General Step 4: Synthesis of Int-6-7

To a solution of Int-6-6 (1 eq) in DCM (5 mL) was added TFA (5 mL). The reaction mixture was stirred at rt for 1 h. LC-MS monitored and the starting material was consumed completely. The mixture was concentrated under vacuum.

MeOH (10 mL) was added followed by K₂CO₃ aqueous to adjust pH to 10. The mixture was stirred at rt overnight. LC-MS monitored and the starting material was consumed completely. HCl aqueous solution was added to adjust pH to 6 and the mixture was extracted with EA (30 mL×3). The combined organic phase was washed with brine, dried over anhydrous Na₂SO₄ and concentrated under vacuum to give Int-6-7.

### General Step 5: Synthesis of Formula 6

To a solution of Int-6-7 (1 eq), Int-6-8 (1.2 er) in DCM (1 mL) and DMF (1 mL) was added DIPEA (3 eq), DMAP (3 eq) and EDCI (1.3 eq). The reaction mixture was stirred at rt overnight. EA (30 mL) was added and the mixture was washed with water (20 mL×2), saturated KH₂PO₄ aqueous solution (20 mL×2), brine, dried over anhydrous Na₂SO₄ and concentrated. The crude residue was purified by prep-HPLC to give Formula 6.

### Example 10: Synthesis of Compound 93

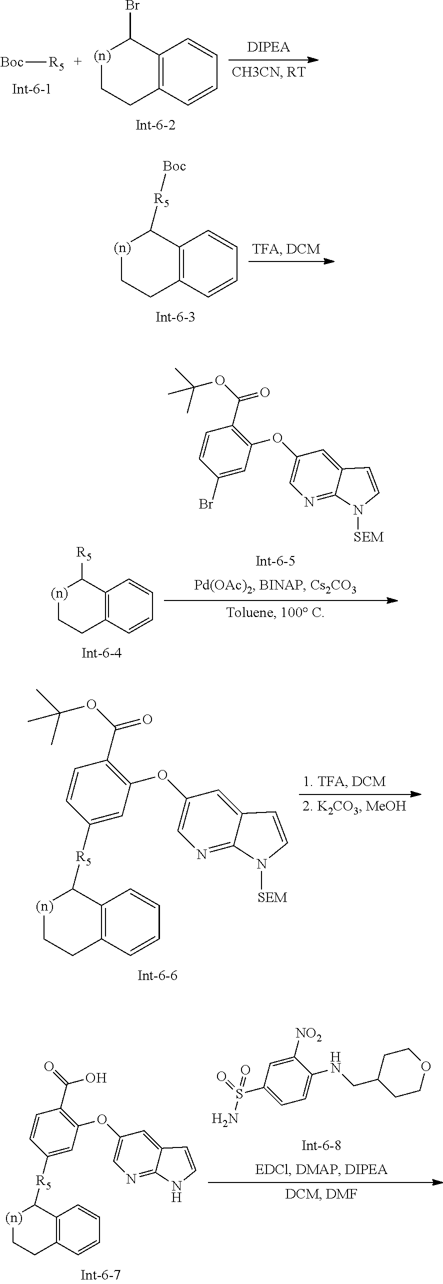

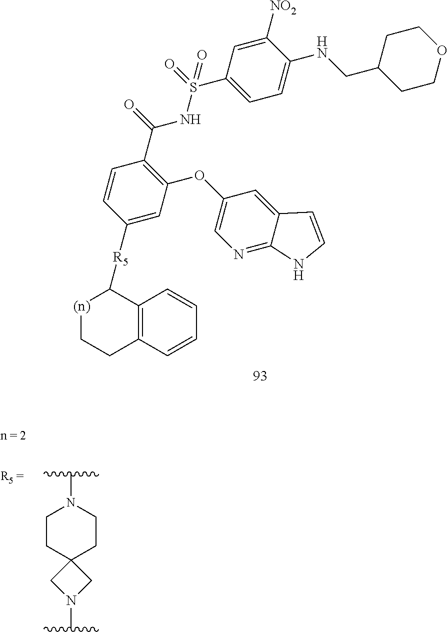

### Step 1: Synthesis of Int-6-3

To a solution of Int-6-1 (200 mg, 0.76 mmol) and Int-6-2 (343 mg, 0.76 mmol) in CH₃CN (8 mL) was added DIEA (196 mg, 1.52 mmol) and the mixture was stirred at rt overnight. EA (30 mL) was added the mixture was washed with water (20 mL×3), brine, dried over anhydrous Na₂SO₄ and concentrated. The crude residue was purified by combine flash (EA/PE=0˜60%) to give Int-6-3 (90 mg, yield 32%). ESI (M+H)⁺ 371.1.

### Step 2: Synthesis of Int-6-4

To a solution of Int-6-3 (90 mg, 0.24 mmol) in DCM (3 mL) was added TFA (1 mL). The reaction mixture was stirred at RT for 1 h. LC-MS monitored and the starting material was consumed completely. The mixture was concentrated under vacuum to give Int-6-4 (66 mg, yield 100%). The crude product was used directly in the next step. ESI (M+H)⁺ 271.1.

### Step 3: Synthesis of Int-6-6

To a solution of Int-6-4 (66 mg, 0.24 mmol), Int-6-5 (127 mg, 0.24 mmol), Pd(OAc)₂ (11 mg, 0.05 mmol), BINAP (46 mg, 0.07 mmol) and Cs₂CO₃ (239 mg, 0.73 mmol) in toluene (5 mL) was heated at 100° C. overnight. LC-MS monitored and desired product produced. H₂O (30 mL) was added and the mixture was extracted with EA (20 mL×3). The combined organic phase was washed with brine, dried over anhydrous Na₂SO₄ and concentrated. The crude residue was purified by combine flash (EA/PE=0˜60%) to give Int-6-6 (90 mg, yield 52%). ESI (M+H)⁺ 708.8.

### Step 4: Synthesis of Int-6-7

To a solution of Int-6-6 (90 mg, 0.13 mmol) in DCM (5 mL) was added TFA (5 mL). The reaction mixture was stirred at rt for 1 h. LC-MS monitored and the starting material was consumed completely. The mixture was concentrated under vacuum.

MeOH (10 mL) was added followed by K₂CO₃ aqueous to adjust pH to 10. The mixture was stirred at rt overnight. LC-MS monitored and the starting material was consumed completely. HCl aqueous solution was added to adjust pH to 6 and the mixture was extracted with EA (30 mL×3). The combined organic phase was washed with brine, dried over anhydrous Na₂SO₄ and concentrated under vacuum to give Int-6-7 (80 mg, yield 121%). The crude product was used in the next step directly. ESI (M+H)⁺ 523.3.

### Step 5: Synthesis of Compound 93

To a solution of Int-6-7 (80 mg, 0.15 mmol), Int-6-8 (58 mg, 0.18 mmol) in DCM (1 mL) and DMF (1 mL) was added DIPEA (0.08 mL, 0.46 mmol), DMAP (56 mg, 0.46 mmol) and EDCI (38 mg, 0.20 mmol). The reaction mixture was stirred at rt overnight. EA (30 mL) was added and the mixture was washed with water (20 mL×2), saturated KH₂PO₄ aqueous solution (20 mL×2), brine, dried over anhydrous Na₂SO₄ and concentrated. The crude residue was purified by Prep-HPLC to give Compound 93 (18.6 mg, yield 15%). ESI (M+H)⁺ 820.3.

### Example 11: Synthesis of Compound 97

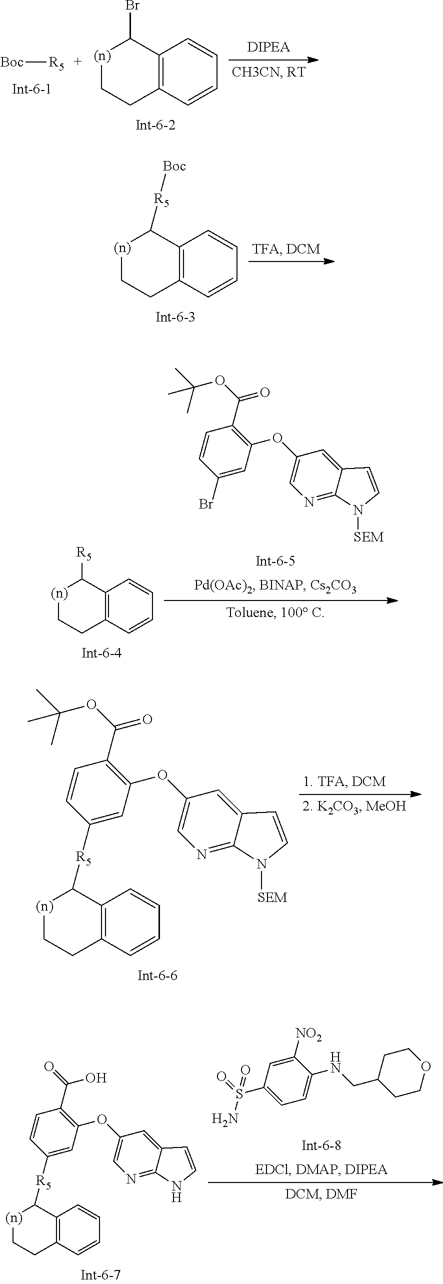

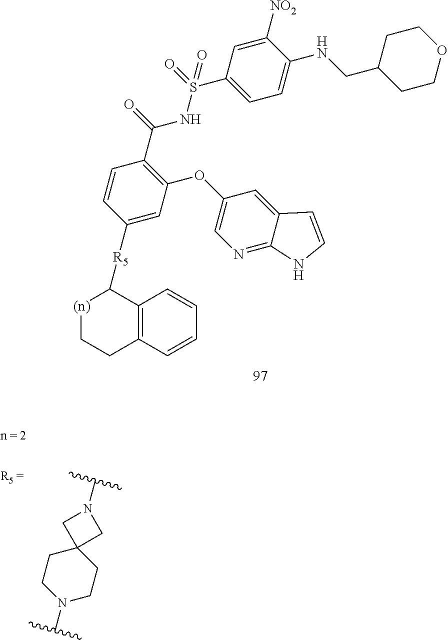

### Step 1: Synthesis of Int-6-3

To a solution of Int-6-1 (320 mg, 1.22 mmol) and Int-6-2 (548 mg, 2.44 mmol) in acetonitrile (10 mL) was added DIPEA (1.0 mL, 6.09 mmol) and the mixture was stirred at 60° C. overnight. EA (30 mL) was added the mixture was washed with water, brine, dried over anhydrous Na₂SO₄ and concentrated. The crude residue was purified by combine flash (EA/PE=0˜60%) to give Int-6-3 (100 mg, yield 22%). ESI (M+H)⁺ 371.1.

### Step 2: Synthesis of Int-6-4

To a solution of Int-6-3 (100 mg, 0.27 mmol) in DCM (3 mL) was added TFA (1 mL). The reaction mixture was stirred at rt for 1 h. LC-MS monitored and the starting material was consumed completely. The mixture was concentrated under vacuum to give Int-6-4 (73 mg, yield 100%). The crude product was used directly in the next step.

### Step 3: Synthesis of Int-6-6

To a solution of Int-6-4 (73 mg, 0.27 mmol), Int-6-5 (168 mg, 0.32 mmol), Pd(OAc)₂ (18 mg, 0.08 mmol), BINAP (67 mg, 0.11 mmol) and Cs₂CO₃ (527 mg, 1.62 mmol) in toluene (5 mL) was heated at 100° C. overnight. LC-MS monitored and desired product produced. H₂O (30 mL) was added and the mixture was extracted with EA (20 mL×3). The combined organic phase was washed with brine, dried over anhydrous Na₂SO₄ and concentrated. The crude residue was purified by combine flash (EA/PE=0˜60%) to give Int-6-6 (90 mg, yield 84%).

### Step 4: Synthesis of Int-6-7

To a solution of Int-6-6 (160 mg, 0.23 mmol) in DCM (5 mL) was added TFA (5 mL). The reaction mixture was stirred at rt for 1 h. LC-MS monitored and the starting material was consumed completely. The mixture was concentrated under vacuum. MeOH (10 mL) was added followed by K₂CO₃ aqueous to adjust pH to 10 The mixture was stirred at rt overnight. LC-MS monitored and the starting material was consumed completely. HCl aqueous solution was added to adjust pH to 6 and the mixture was extracted with EA (30 mL×3). The combined organic phase was washed with brine, dried over anhydrous Na₂SO₄ and concentrated under vacuum to give Int-6-7 (80 mg, yield 68%). ESI (M+H)⁺ 523.2. The crude product was used in the next step directly.

### Step 5: Synthesis of Compound 97

To a solution of Int-6-7 (80 mg, 0.15 mmol), Int-6-8 (58 mg, 0.18 mmol) in DCM (1 mL) and DMF (1 mL) was added DIPEA (0.08 mL, 0.46 mmol), DMAP (56 mg, 0.46 mmol) and EDCI (38 mg, 0.20 mmol). The reaction mixture was stirred at rt overnight. EA (30 mL) was added and the mixture was washed with water (20 mL×2), saturated KH₂PO₄ aqueous solution (20 mL×2), brine, dried over anhydrous Na₂SO₄ and concentrated. The crude residue was purified by Prep-HPLC to give Compound 97 (19.7 mg, yield 16%). ESI (M+H)⁺ 820.2.

The following compounds were prepared according to the above described methods using different starting materials.

| Compound | | MS(ESI): m/z |
|---|---|---|
| No. | IUPAC Name | (M + H⁺) |
| 91 | 2-((1H-pyrrolo[2,3-b]pyridin-5-yl)oxy)-N-((3-nitro-4- | 792.3 |
| | (((tetrahydro-2H-pyran-4- | |
| | yl)methyl)amino)phenyl)sulfonyl)-4-(6-(1,2,3,4- | |
| | tetrahydronaphthalen-1-yl)-2,6-diazaspiro[3.4]octan-2- | |
| | yl)benzamide | |
| 92 | 2-((1H-pyrrolo[2,3-b]pyridin-5-yl)oxy)-N-((3-nitro-4- | 806.3 |
| | (((tetrahydro-2H-pyran-4- | |
| | yl)methyl)amino)phenyl)sulfonyl)-4-(7-(1,2,3,4- | |
| | tetrahydronaphthalen-1-yl)-2,7-diazaspiro[3.5]nonan- | |
| | 2-yl)benzamide | |
| 94 | 2-((1H-pyrrolo[2,3-b]pyridin-5-yl)oxy)-N-((3-nitro-4- | 806.3 |
| | (((tetrahydro-2H-pyran-4- | |
| | yl)methyl)amino)phenyl)sulfonyl)-4-(2-(1,2,3,4- | |
| | tetrahydronaphthalen-1-yl)-2,7-diazaspiro[3.5]nonan- | |
| | 7-yl)benzamide | |
| 96 | 2-((1H-pyrrolo[2,3-b]pyridin-5-yl)oxy)-N-((3-nitro-4- | 777.8 |
| | (((tetrahydro-2H-pyran-4- | |
| | yl)methyl)amino)phenyl)sulfonyl)-4-(6-(1,2,3,4- | |
| | tetrahydronaphthalen-1-yl)-2,6-diazaspiro[3.3]heptan- | |
| | 2-yl)benzamide | |
| 95 | 2-((1H-pyrrolo[2,3-b]pyridin-5-yl)oxy)-N-((3-nitro-4- | 792.3 |
| | (((tetrahydro-2H-pyran-4- | |
| | yl)methyl)amino)phenyl)sulfonyl)-4-(6-(6,7,8,9- | |
| | tetrahydro-5H-benzo[7]annulen-5-yl)-2,6- | |
| | diazaspiro[3.3]heptan-2-yl)benzamide | |
| 98 | 2-((1H-pyrrolo[2,3-b]pyridin-5-yl)oxy)-N-((3-nitro-4- | 806.3 |
| | (((tetrahydro-2H-pyran-4- | |
| | yl)methyl)amino)phenyl)sulfonyl)-4-(6-(6,7,8,9- | |
| | tetrahydro-5H-benzo[7]annulen-5-yl)-2,6- | |
| | diazaspiro[3.4]octan-2-yl)benzamide | |

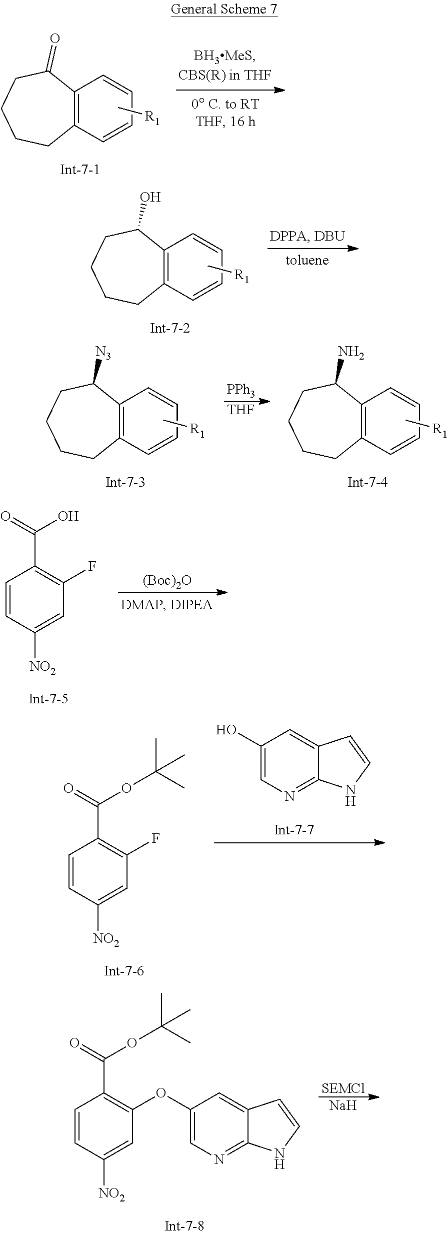

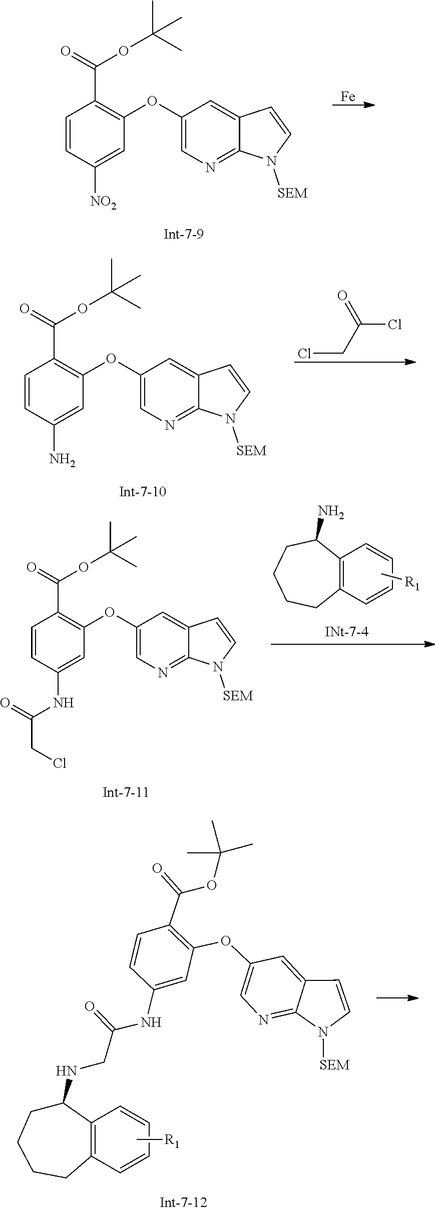

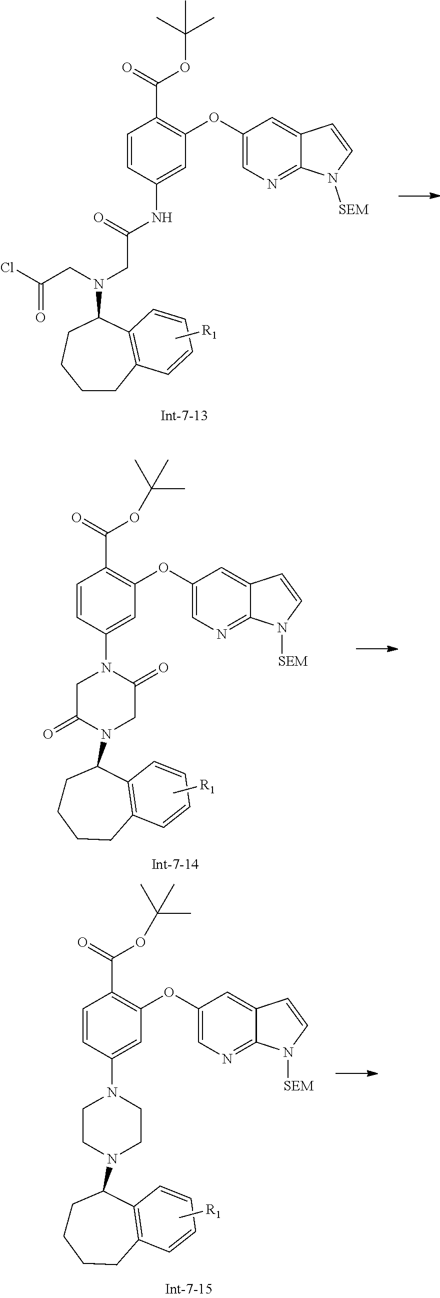

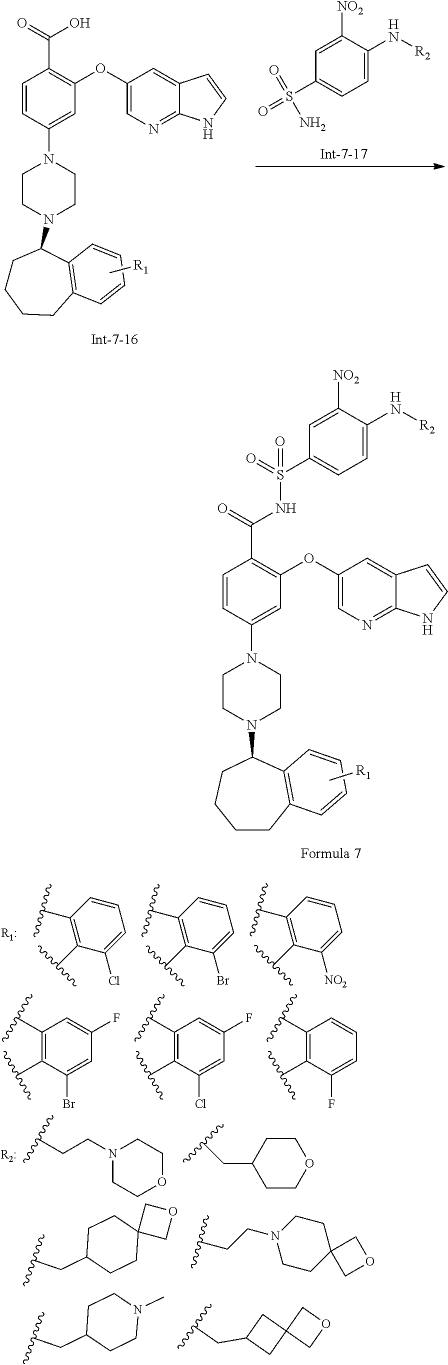

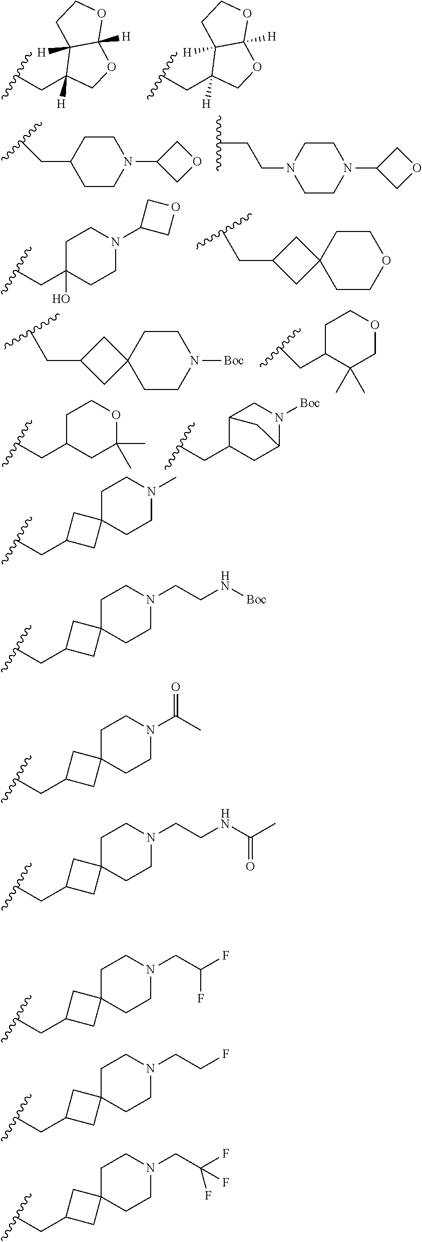

### General Step 1: Synthesis of Int-7-2

A solution of 2M BH₃-Me₂S in THE (206.0 mmol, 1.5 eq) and 1M (R)-3,3-Diphenyl-1-methylpyrrolidino[1,2-c]-1,3,2-oxazaborole in toluene (274.0 mmol, 2.0 eq) was stirred at 0° C. for 1 h. Then Int-7-1 (137 mmol, 1.0 eq) in THE (100 mL) was added and the reaction mixture was stirred 0° C. for 2 h. Methanol was added to quench the reaction. The solvent was removed under vacuum. H₂O (300 mL) was added and the mixture was extracted with DCM (200 mL×3). The combined organic phase was washed with brine, dried over anhydrous Na₂SO₄ and concentrated under vacuum. The crude residue was purified by combine flash (EA/PE=0˜30%) to give Int-7-2.

### General Step 2: Synthesis of Int-7-3

To a solution of Int-7-2 (12.44 mmol, 1.0 eq) in toluene (20 mL) was added DPPA (24.88 mmol, 2 eq) and DBU (18.66 mmol, 1.5 eq). The reaction mixture was stirred under N2 atmosphere at 50° C. for 4 hours. H₂O (50 mL) was added and the mixture was extracted with DCM (30 mL×3). The combined organic phase was washed with brine, dried over anhydrous Na₂SO₄ and concentrated under vacuum. The crude residue was purified by combine flash (PE=100%) to give Int-7-3.

### General Step 3: Synthesis of Int-7-4

Method I: To a solution of Int-7-3 (15.79 mmol, 1.0 eq) in THE (50 mL) and H₂O (5 mL) was added PPh₃ (31.58 mmol, 2.0 eq). The reaction mixture was stirred at 50° C. overnight. H₂O (100 mL) was added and the mixture was extracted with DCM (50 mL×3). The combined organic phase was washed with brine, dried over anhydrous Na₂SO₄ and concentrated under vacuum. The crude residue was dissolved in DCM (60 mL) and concentrated HCl (4 mL) was added. The solution was filtrated to give Int-7-4.

Method II. To a solution of Int-7-3 (4.51 mmol, 1.0 eq) in methanol (20 mL) was added NiCl₂ (4.96 mmol, 1.1 eq) and NaBH₄ (6.76 mmol, 1.5 eq) at 0° C. and the reaction mixture was stirred at 0° C. for 2 h. H₂O (20 mL) was added and the mixture was extracted with EA (20 mL×3). The combined organic phase was washed with brine, dried over anhydrous Na₂SO₄ and concentrated under vacuum. The crude residue was purified by combine flash (ME/DCM=0˜10%) to give Int-7-4.

### General Step 4: Synthesis of Int-7-6

To a stirred solution of Int-7-5 (20.00 g, 108 mmol, 1.0 eq) in DCM (200 mL) was added DMAP (6.60 g, 54.02 mmol, 0.5 eq), DIPEA (54 mL, 324 mmol, 3.0 eq) and (Boc)₂O (35.37 g, 162 mmol, 1.5 eq). The mixture was stirred at rt overnight. It was quenched with aqueous HCl solution (50 mL), and the aqueous solution was extracted with DCM (50 mL×3). The combined organic extracts were washed with brine (50 mL), dried over anhydrous sodium sulfate and concentrated under the reduced pressure. The residue was purified by combine flash (EA/PE=0˜15%) to give Int-7-6 (22 g, yield 84%).

### General Step 5: Synthesis of Int-7-8

To a stirred solution of Int-7-6 (21.00 g, 87.06 mmol, 1.0 eq), Int-7-7 (17.52 g, 130.59 mmol, 1.5 eq) in NMP (200 mL) was added Cs₂CO₃ (42.55 g, 130.59 mmol, 1.5 eq). The mixture was stirred at rt for 15 h under N₂ and LC-MS shows total consumption of Int-7-6. The reaction mixture was filtered. The filtrate was diluted with water (200 mL) and was extracted with EA (100 mL×3). The combined organic extracts were washed with brine (100 mL×7), dried it over sodium sulfate and concentrated under reduced pressure. The residue was purified by combine flash (EA/PE=0˜30%) to give Int-7-8 (21 g, 68%). MS(ESI): m/z 356.0 (M+H⁺).

### General Step 6: Synthesis of Int-7-9

To a stirred solution of Int-7-8 (21.00 g, 59.10 mmol, 1.0 eq) in THE (210 mL) was added 60% NaH (3.07 g, 76.83 mmol, 1.5 eq) at 0° C. The mixture was stirred at 0° C. for 1 h and then SEMCl (12.81 g, 76.83 mmol, 1.5 eq) was added. The mixture was stirred at rt for 2 h and LC-MS shows the total consumption of Int-7-8. It was quenched by water (20 mL) and extracted with EA (3×100 mL). The combined organic layer was washed brine (1×50 mL), dried over sodium sulfate and concentrated under reduced pressure. The residue was purified by combine flash (EA/PE=0˜30%) to give Int-7-9 (25 g, yield 87%). MS(ESI): m/z 486.2 (M+H⁺).

### General Step 7: Synthesis of Int-7-10

To a stirred solution of Int-7-9 (23.00 g, 47.36 mmol, 1.0 eq) in Ethanol (200 mL) and saturated NH₄Cl aqueous solution (80 mL) was added Fe (13.22 g, 236.81 mmol, 5.0 eq). The resulting mixture was stirred at 90° C. under N₂ for 3 h. The reaction mixture was filtered and filtrate was diluted with water (200 mL) and extracted with EtOAc (3×100 mL). The combined organic extracts were washed with brine (1×50 mL), dried over sodium sulfate and concentrated under reduced pressure. The residue was purified by combine flash (EA/PE=0˜40%) to give Int-7-10 (21 g, yield 97%). MS(ESI): m/z 456.2 (M+H⁺).

### General Step 8: Synthesis of Int-7-11

To a stirred solution of Int-7-10 (21 g, 46.09 mmol, 1.0 eq) in DCM (200 mL) was added and TEA (25.6 mL, 184.36 mmol, 4.0 eq) and 2-chloroacetyl chloride (10.41 g, 92.18 mmol, 2.0 eq) at 0° C. The resulting mixture was stirred at rt for 2 h and LC-MS shows the total consumption of Int-7-10. It was quench by aqueous NH₄HCO₃ solution and extracted with DCM (3×100 mL). The combined organic layer was washed with brine (1×50 mL), dried over sodium sulfate and concentrated under reduced pressure. The residue was purified by flash chromatography (EA/PE=0˜60%) to give Int-7-11 (22 g, 90%). MS(ESI): m/z 532.1 (M+H⁺).

### General Step 9: Synthesis of Int-7-12

To a solution of Int-7-4 (1.13 mmol, 1.0 eq), Int-7-11 (1.13 mmol, 1.0 eq) in acetonitrile (30 mL) was added NaI (3.38 mmol, 3.0 eq), K₂CO₃ (3.38 mmol, 3.0 eq). The mixture was heated at 90° C. for 5 h. H₂O (40 mL) was added and the mixture was extracted with EA (20 mL×3). The combined organic phase was washed with brine, dried over anhydrous Na₂SO₄ and concentrated under vacuum. The crude residue was purified by combine flash (EA/PE=0˜40%) to give Int-7-12.

### General Step 10: Synthesis of Int-7-13

To a solution of Int-7-12 (1.01 mmol, 1.0 eq) in DCM (15 mL) was added TEA (4.04 mmol, 4.0 eq) and 2-chloroacetyl chloride (2.02 mmol, 2.0 eq) at 0° C. The reaction mixture was stirred at rt for 1 h. The organic phase was washed with saturated NaHCO₃ (15 mL×1), brine, dried over anhydrous Na₂SO₄ and concentrated under vacuum. The crude residue was purified by combine flash (EA/PE=0˜40%) to give Int-7-13.

### General Step 11: Synthesis of Int-7-14

To a solution of Int-7-13 (0.98 mmol, 1.0 eq) in acetonitrile (15 mL) was added NaI (2.95 mmol, 3.0 eq), K₂CO₃ (2.95 mmol, 3.0 eq). The reaction mixture was heated at 90° C. for 4 h. EA (50 mL) was added and the organic phase was washed with H₂O (20 mL×1), brine, dried over anhydrous Na₂SO₄ and concentrated under vacuum. The crude residue was purified by combine flash (EA/PE=0˜40%) to give Int-7-14.

### General Step 12: Synthesis of Int-7-15

To a solution of Int-7-14 (1.0 mmol, 1.0 eq) in THF (10 mL) was added 1M BH₃·THF (10.0 mmol, 10.0 eq) and the mixture was stirred at rt for 2 h. MeOH (30 mL) was added slowly and the mixture was stirred at 60° C. overnight. The solvent was removed under vacuum. H₂O (40 mL) was added and the mixture was extracted with EA (20 mL×3). The combined organic phase was washed with brine, dried over anhydrous Na₂SO₄ and concentrated under vacuum. The crude residue was purified by combine flash (EA/PE=0˜40%) to give Int-7-15.

### General Step 13: Synthesis of Int-7-16

To a solution of Int-7-15 (0.27 mmol) in DCM (2 mL) was added TFA (2 mL). The reaction mixture was stirred at rt for 2 h. LC-MS monitored and the starting material was consumed completely. The mixture was concentrated under vacuum. The residue was dissolved in MeOH (8 mL) and K₂CO₃ aqueous solution was added to adjust pH>8. The mixture was stirred at rt overnight. 1M HCl aqueous solution was added to adjust the pH to 5 and the mixture was extracted with DCM (20 mL×3). The combined organic phase was washed with brine, dried over anhydrous Na₂SO₄ and concentrated to give Int-7-16.

### General Step 14: Synthesis of Formula 7

To a solution of Int-7-16 (1.0 mmol, 1.0 eq) and Int-7-17 (1.0 mmol, 1.0 eq) in DMF (5 mL) was added DIPEA (3.0 mmol, 3.0 eq), DMAP (3.0 mmol, 3.0 eq) and EDCI (1.3 mmol, 1.3 eq). The reaction mixture was stirred at rt overnight. The crude mixture was purified by prep-HPLC to give Formula 7.

### Example 12: Synthesis of Compound 18A

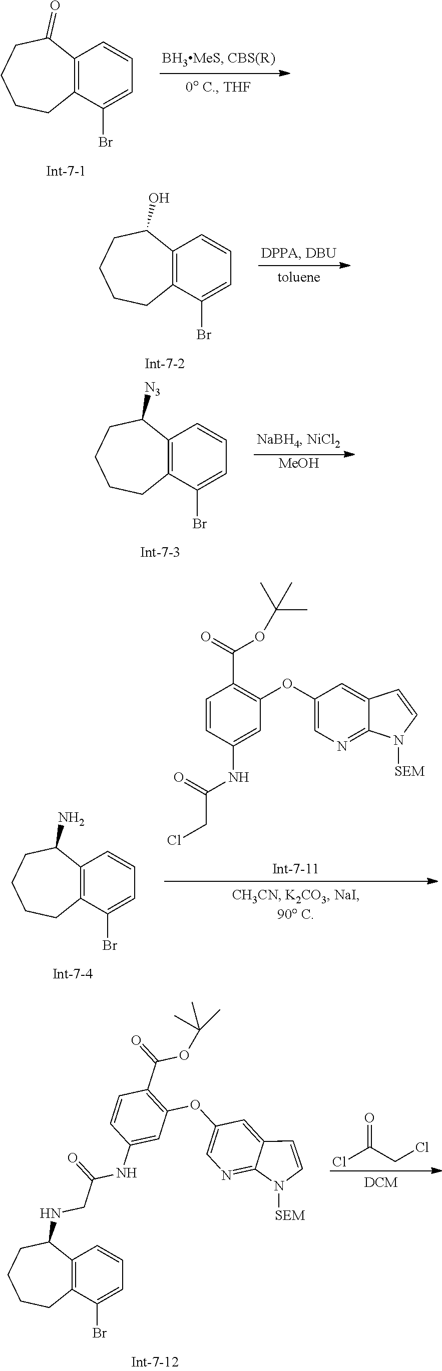

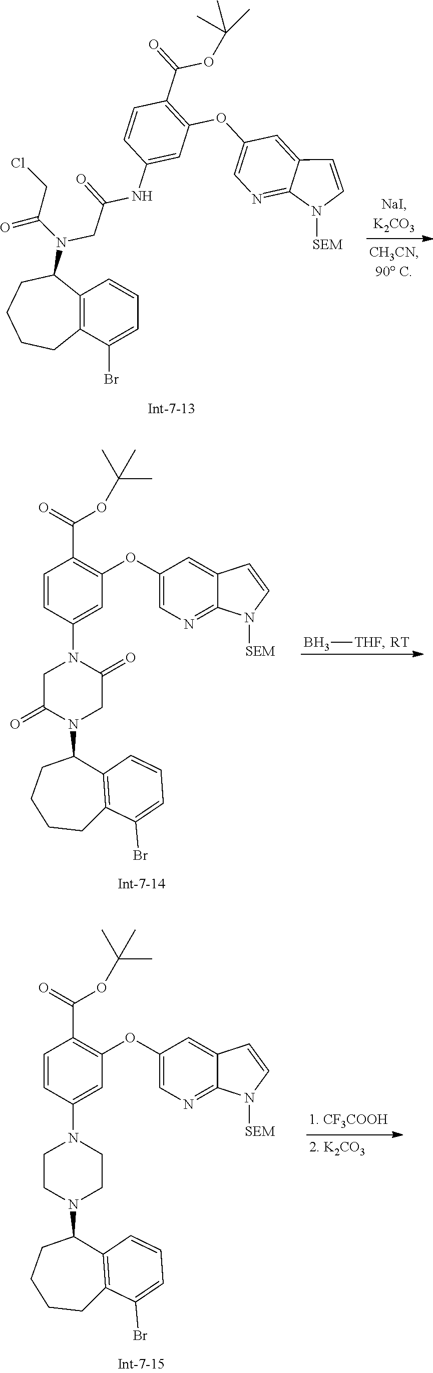

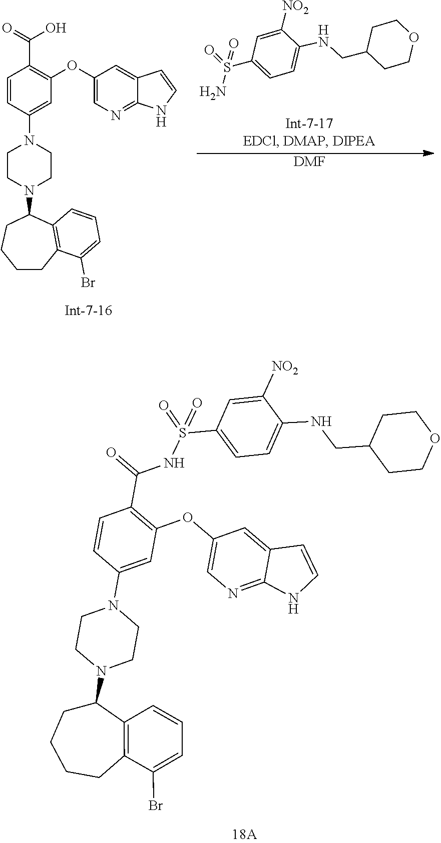

### Step 1: Synthesis of (S)-1-bromo-6,7,8,9-tetrahydro-5H-benzo[7]annulen-5-ol (Int-7-2)

A solution of 2M BH₃Me₂S in THE (103 mL, 206.0 mmol, 1.5 eq) and 1M (R)-3,3-Diphenyl-1-methylpyrrolidino[1,2-c]-1,3,2-oxazaborole in toluene (274 mL, 274.0 mmol, 2.0 eq) was stirred at 0° C. for 1 h. Then the mixture of 1-bromo-6,7,8,9-tetrahydro-5H-benzo[7]annulen-5-one (Int-7-1) and 3-bromo-6,7,8,9-tetrahydro-5H-benzo[7]annulen-5-one (unseparated byproduct in the previous step) (32.8 g, 137.2 mmol, 1.0 eq) in THE (100 mL) was added and the reaction mixture was stirred 0° C. for 2 h. Methanol was added to quench the reaction. The solvent was removed under vacuum. H₂O (300 mL) was added and the mixture was extracted with DCM (200 mL×3). The combined organic phase was washed with brine, dried over anhydrous Na₂SO₄ and concentrated under vacuum. The crude residue was purified by combine flash (EA/PE=0-30%) to give (S)-1-bromo-6,7,8,9-tetrahydro-5H-benzo[7]annulen-5-ol (Int-7-2) (6.6 g, yield 20%). MS(ESI): m/z 223.1 (M-OH).

### Step 2: Synthesis of (R)-5-azido-1-bromo-6,7,8,9-tetrahydro-5H-benzo[7]annulene (Int-7-3)

To a solution of S)-1-bromo-6,7,8,9-tetrahydro-5H-benzo[7]annulen-5-ol (Int-7-2) (3.0 g, 12.44 mmol, 1.0 eq) in toluene (20 mL) was added DPPA (5.4 mL, 24.88 mmol, 2.0 eq) and DBU (2.8 mL, 18.66 mmol, 1.5 eq). The reaction mixture was stirred under N₂ atmosphere at 50° C. for 4 hours. H₂O (50 mL) was added and the mixture was extracted with DCM (30 mL×3). The combined organic phase was washed with 1 M HCl aqueous solution (30 mL×1), brine, dried over anhydrous Na₂SO₄ and concentrated under vacuum. The crude residue was purified by combine flash (PE=100%) to give (R)-5-azido-1-bromo-6,7,8,9-tetrahydro-5H-benzo[7]annulene (Int-7-3) (2.4 g, yield 86%). MS(ESI): m/z 223.1 (M-N₃).

### Step 3: Synthesis of (R)-1-bromo-6,7,8,9-tetrahydro-5H-benzo[7]annulen-5-amine (Int-7-4)

To a solution of (R)-5-azido-1-bromo-6,7,8,9-tetrahydro-5H-benzo[7]annulene (Int-7-3) (2.4 g, 9.02 mmol, 1.0 eq) in methanol (30 mL) was added NiCl₂ (1.29 g, 9.92 mmol, 1.1 eq) and NaBH₄ (514 mg, 13.53 mmol, 1.5 eq) at 0° C. and the reaction mixture was stirred at 0° C. for 2 h. H₂O (100 mL) was added and the mixture was extracted with EA (50 mL×3). The combined organic phase was washed with brine, dried over anhydrous Na₂SO₄ and concentrated under vacuum. The crude residue was purified by combine flash (ME/DCM=0˜10%) to give (R)-1-bromo-6,7,8,9-tetrahydro-5H-benzo[7]annulen-5-amine (Int-7-4) (1.88 g, yield 87%). MS(ESI): m/z 223.1 (M-NH₂).

### Step 4: Synthesis of tert-butyl (R)-4-(2-((1-bromo-6,7,8,9-tetrahydro-5H-benzo[7]annulen-5-yl)amino)acetamido)-2-((1-((2-(trimethylsilyl)ethoxy)methyl)-1H-pyrrolo[2,3-b]pyridin-5-yl)oxy)benzoate (Int-7-12)

To a solution of (R)-1-bromo-6,7,8,9-tetrahydro-5H-benzo[7]annulen-5-amine (Int-7-4) (600 mg, 1.13 mmol, 1.0 eq), tert-butyl 4-(2-chloroacetamido)-2-((1-((2-(trimethylsilyl)ethoxy)methyl)-1H-pyrrolo[2,3-b]pyridin-5-yl)oxy)benzoate (Int-7-11) (271 mg, 1.13 mmol, 1.0 eq), NaI (507 mg, 3.38 mmol, 3.0 eq), K₂CO₃ (468 mg, 3.38 mmol, 3.0 eq) in acetonitrile (30 mL) was heated at 90° C. for 5 h. H₂O (40 mL) was added and the mixture was extracted with EA (20 mL×3). The combined organic phase was washed with brine, dried over anhydrous Na₂SO₄ and concentrated under vacuum. The crude residue was purified by combine flash (EA/PE=0˜40%) to give tert-butyl (R)-4-(2-((1-bromo-6,7,8,9-tetrahydro-5H-benzo[7]annulen-5-yl)amino)acetamido)-2-((1-((2-(trimethylsilyl)ethoxy)methyl)-1H-pyrrolo[2,3-b]pyridin-5-yl)oxy)benzoate (Int-7-12) (740 mg, yield 89%). MS(ESI): m/z 735.0 (M+H⁺).

### Step 5: Synthesis of tert-butyl (R)-4-(2-(N-(1-bromo-6,7,8,9-tetrahydro-5H-benzo[7]annulen-5-yl)-2-chloroacetamido)acetamido)-2-((1-((2-(trimethylsilyl)ethoxy)methyl)-1H-pyrrolo[2,3-b]pyridin-5-yl)oxy)benzoate (Int-7-13)

To a solution of tert-butyl (R)-4-(2-((1-bromo-6,7,8,9-tetrahydro-5H-benzo[7]annulen-5-yl)amino)acetamido)-2-((1-((2-(trimethylsilyl)ethoxy)methyl)-1H-pyrrolo[2,3-b]pyridin-5-yl)oxy)benzoate (Int-7-12) (740 mg, 1.01 mmol, 1.0 eq) in DCM (15 mL) was added TEA (0.56 mL, 4.04 mmol, 4.0 eq) and 2-chloroacetyl chloride (0.16 mL, 2.02 mmol, 2.0 eq). The reaction mixture was stirred at RT for 1 h. The organic phase was washed with saturated NaHCO₃ (15 mL×1), brine, dried over anhydrous Na₂SO₄ and concentrated under vacuum. The crude residue was purified by combine flash (EA/PE=0˜40%) to give tert-butyl (R)-4-(2-(N-(1-bromo-6,7,8,9-tetrahydro-5H-benzo[7]annulen-5-yl)-2-chloroacetamido)acetamido)-2-((1-((2-(trimethylsilyl)ethoxy)methyl)-1H-pyrrolo[2,3-b]pyridin-5-yl)oxy)benzoate (Int-7-13) (800 mg, yield 98%). MS(ESI): m/z 811.0 (M+H⁺).

### Step 6: Synthesis of tert-butyl (R)-4-(4-(1-bromo-6,7,8,9-tetrahydro-5H-benzo[7]annulen-5-yl)-2,5-dioxopiperazin-1-yl)-2-((1-((2-(trimethylsilyl)ethoxy)methyl)-1H-pyrrolo[2,3-b]pyridin-5-yl)oxy)benzoate (Int-7-14)

To a solution of tert-butyl (R)-4-(2-(N-(1-bromo-6,7,8,9-tetrahydro-5H-benzo[7]annulen-5-yl)-2-chloroacetamido)acetamido)-2-((1-((2-(trimethylsilyl)ethoxy)methyl)-1H-pyrrolo[2,3-b]pyridin-5-yl)oxy)benzoate (Int-7-13) (800 mg, 0.98 mmol, 1.0 eq), NaI (443 mg, 2.95 mmol, 3.0 eq), K₂CO₃ (408 mg, 2.95 mmol, 3.0 eq) in acetonitrile (15 mL) was heated at 90° C. for 4 h. EA (50 mL) was added and the organic phase was washed with H₂O (20 mL×1), brine, dried over anhydrous Na₂SO₄ and concentrated under vacuum. The crude residue was purified by combine flash (EA/PE=0˜40%) to give tert-butyl (R)-4-(4-(1-bromo-6,7,8,9-tetrahydro-5H-benzo[7]annulen-5-yl)-2,5-dioxopiperazin-1-yl)-2-((1-((2-(trimethylsilyl)ethoxy)methyl)-1H-pyrrolo[2,3-b]pyridin-5-yl)oxy)benzoate (Int-7-14) (730 mg, yield 96%). MS(ESI): m/z 775.0 (M+H⁺).

### Step 7: Synthesis of tert-butyl (R)-4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-7-15)

To a solution of tert-butyl (R)-4-(4-(1-bromo-6,7,8,9-tetrahydro-5H-benzo[7]annulen-5-yl)-2,5-dioxopiperazin-1-yl)-2-((1-((2-(trimethylsilyl)ethoxy)methyl)-1H-pyrrolo[2,3-b]pyridin-5-yl)oxy)benzoate (Int-7-14) (730 mg, 0.94 mmol, 1.0 eq) in THE (10 mL) was added 1M BH3·THF (10.0 mL, 10.0 mmol, 10.0 eq) and the mixture was stirred at RT for 2 h. MeOH (30 mL) was added slowly and the mixture was stirred at 60° C. overnight. The solvent was removed under vacuum. H₂O (30 mL) was added slowly and the mixture was extracted with EA (30 mL×3). The combined organic phase was wished with brine, dried over anhydrous Na₂SO₄ and concentrated under vacuum. The crude residue was purified by combine flash (EA/PE=0˜40%) to give tert-butyl (R)-4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-7-15) (440 mg, yield 64%). MS(ESI): m/z 747.2 (M+H⁺).

### Step 8: Synthesis of (R)-2-((1H-pyrrolo[2,3-b]pyridin-5-yl)oxy)-4-(4-(1-bromo-6,7,8,9-tetrahydro-5H-benzo[7]annulen-5-yl)piperazin-1-yl)benzoic acid (Int-7-16)

To a solution of tert-butyl (R)-4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-7-15) (200 mg, 0.27 mmol) in DCM (2 mL) was added TFA (2 mL). The reaction mixture was stirred at RT for 2 h. LC-MS monitored and the starting material was consumed completely. The mixture was concentrated under vacuum. The residue was dissolved in MeOH (8 mL) and added K₂CO₃ aqueous to adjust pH>8. The mixture was stirred at RT overnight. LC-MS monitored and the starting material was consumed completely. 1M HCl aqueous solution was added to adjust the pH to 5 and the mixture was extracted wtih DCM (20 mL×3). The combined organic phase was washed with brine, dried over anhydrous Na₂SO₄ and concentrated to give (R)-2-((1H-pyrrolo[2,3-b]pyridin-5-yl)oxy)-4-(4-(1-bromo-6,7,8,9-tetrahydro-5H-benzo[7]annulen-5-yl)piperazin-1-yl)benzoic acid (Int-7-16) (150 mg, yield 100%). The crude product was used in the next step directly. MS(ESI): m/z 561.0 (M+H⁺).

### Step 9: Synthesis of (R)-2-((1H-pyrrolo[2,3-b]pyridin-5-yl)oxy)-4-(4-(1-bromo-6,7,8,9-tetrahydro-5H-benzo[7]annulen-5-yl)piperazin-1-yl)-N-((3-nitro-4-(((tetrahydro-2H-pyran-4-yl)methyl)amino)phenyl)sulfonyl)benzamide (Compound 18A)

To a solution of (R)-2-((1H-pyrrolo[2,3-b]pyridin-5-yl)oxy)-4-(4-(1-bromo-6,7,8,9-tetrahydro-5H-benzo[7]annulen-5-yl)piperazin-1-yl)benzoic acid (Int-7-16) (170 mg, 0.31 mmol, 1.0 eq), 3-nitro-4-(((tetrahydro-2H-pyran-4-yl)methyl)amino)benzenesulfonamide (Int-7-17) (99 mg, 0.31 mmol, 1.0 eq) in DMF (2 mL) was added DIPEA (0.15 mL, 0.91 mmol, 3.0 eq), DMAP (74 mg, 0.61 mmol, 3.0 eq) and EDCI (75 mg, 0.39 mmol, 1.3 eq). The reaction mixture was stirred at rt overnight. EA (30 mL) was added and the mixture was washed with water (20 mL×3), brine, dried over anhydrous Na₂SO₄ and concentrated. The crude residue was purified by prep-HPLC to give (R)-2-((1H-pyrrolo[2,3-b]pyridin-5-yl)oxy)-4-(4-(1-bromo-6,7,8,9-tetrahydro-5H-benzo[7]annulen-5-yl)piperazin-1-yl)-N-((3-nitro-4-(((tetrahydro-2H-pyran-4-yl)methyl)amino)phenyl)sulfonyl)benzamide (Compound 18A) (72.0 mg, yield 28%). MS(ESI): m/z 858.2 (M+H⁺).

### Example 13: Synthesis of Compound 73

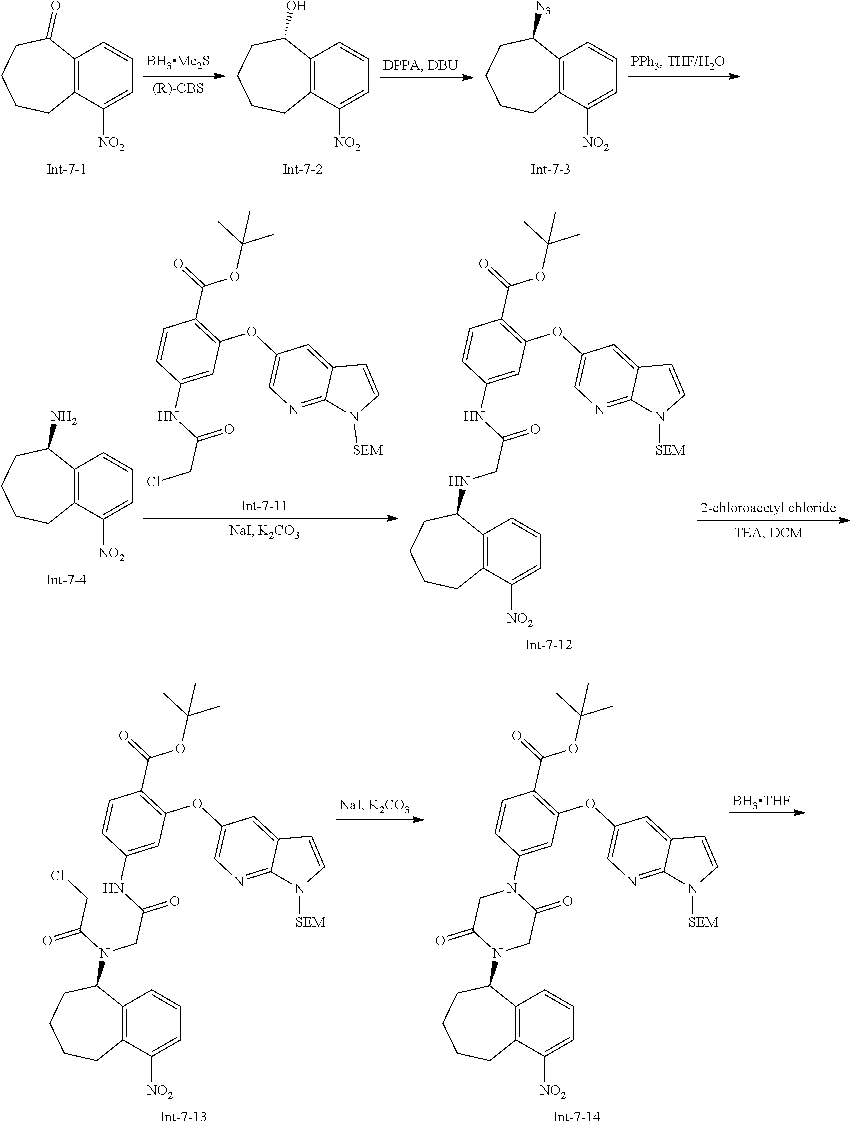

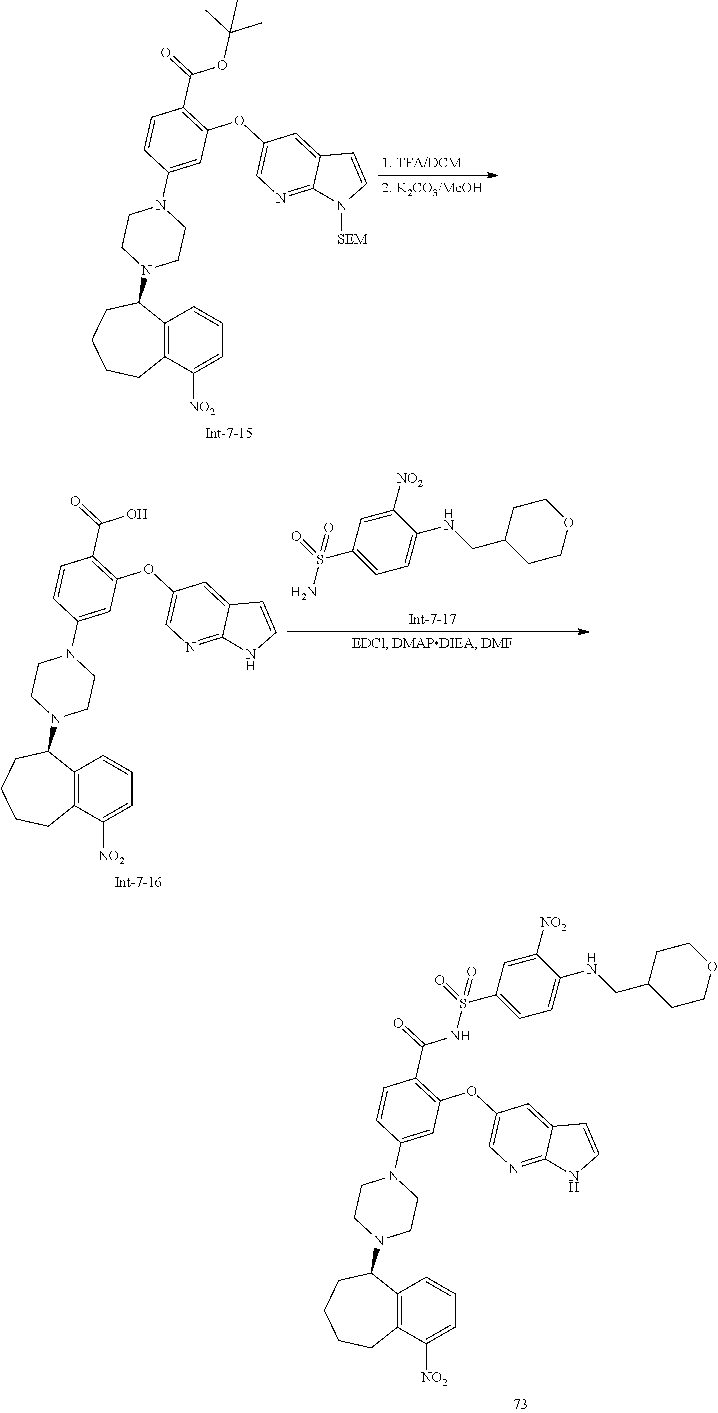 [SMILES: CC(C)(C)OC(=O)c1ccc(N2CCN([C@@H]3CCCCc4c3cccc4[N+](=O)[O-])CC2)cc1Oc1cnc2c(ccn2COCC[Si](C)(C)C)c1 ; O=C(O)c1ccc(N2CCN([C@@H]3CCCCc4c3cccc4[N+](=O)[O-])CC2)cc1Oc1cnc2[nH]ccc2c1 ; NS(=O)(=O)c1ccc(NCC2CCOCC2)c([N+](=O)[O-])c1 ; O=C(NS(=O)(=O)c1ccc(NCC2CCOCC2)c([N+](=O)[O-])c1)c1ccc(N2CCN([C@@H]3CCCCc4c3cccc4[N+](=O)[O-])CC2)cc1Oc1cnc2[nH]ccc2c1]

### Step 1: Synthesis of (S)-1-nitro-6,7,8,9-tetrahydro-5H-benzo[7]annulen-5-ol (Int-7-2)

A solution of 2M BH3 Me₂S in THE (18.3 mL, 36.58 mmol, 1.5 eq) and 1M (R)-3,3-Diphenyl-1-methylpyrrolidino[1,2-c]-1,3,2-oxazaborole in toluene (49 mL, 48.78 mmol, 2.0 eq) was stirred at 0° C. for 1 h. Then 1-nitro-6,7,8,9-tetrahydro-5H-benzo[7]annulen-5-one (Int-7-1) (5.0 g, 24.39 mmol, 1.0 eq) in THE (20 mL) was added and the reaction mixture was stirred 0° C. for 2 hours. A little methanol was added to quench the reaction. H₂O (100 mL) was added and the mixture was extracted with DCM (100 mL×3). The combined organic phase was washed with brine, dried over anhydrous Na₂SO₄ and concentrated under vacuum. The crude residue was purified by combine flash (EA/PE=0˜40%) to give (S)-1-nitro-6,7,8,9-tetrahydro-5H-benzo[7]annulen-5-ol (Int-7-2) (5.0 g, yield 99%). MS(ESI): m/z 190.1 (M-OH).

### Step 2: Synthesis of (R)-5-azido-1-nitro-6,7,8,9-tetrahydro-5H-benzo[7]annulene (Int-7-3)

To a solution of (S)-1-nitro-6,7,8,9-tetrahydro-5H-benzo[7]annulen-5-ol (Int-7-2) (5.0 g, 25.12 mmol, 1.0 eq) in toluene (50 mL) was added DPPA (13.8 g, 50.24 mmol, 2.0 eq) and DBU (5.7 g, 37.68 mmol, 1.5 eq). The reaction mixture was stirred under N₂ atmosphere at 50° C. for 4 hours. H₂O (50 mL) was added and the mixture was extracted with DCM (30 mL×3). The combined organic phase was washed with 1M HCl aqueous solution (30 mL×1), brine, dried over anhydrous Na₂SO₄ and concentrated under vacuum. The crude residue was purified by combine flash (EA/PE=0˜40%) to give (R)-5-azido-1-nitro-6,7,8,9-tetrahydro-5H-benzo[7]annulene (Int-7-3) (4.3 g, yield 77%). MS(ESI): m/z 190.1 (M-N₃).

### Step 3: Synthesis of (R)-1-nitro-6,7,8,9-tetrahydro-5H-benzo[7]annulen-5-amine hydrochloride (Int-7-4)

To a solution of (R)-5-azido-1-nitro-6,7,8,9-tetrahydro-5H-benzo[7]annulene (Int-7-3) (4.0 g, 17.22 mmol, 1.0 eq) in THF (60 mL) and ITO (6 mL) was added PPh₃ (9.04 g, 34.45 mmol, 2.0 eq). The reaction mixture was stirred under N₂ atmosphere at 50° C. for 16 hours. H₂O (50 mL) was added and the mixture was extracted with DCM (30 mL×3). The combined organic phase was washed with brine, dried over anhydrous Na₂SO₄ and then concentrated HCl solution was added dropwise, filtrated. The residue was washed with DCM, dried to give (R)-1-nitro-6,7,8,9-tetrahydro-5H-benzo[7]annulen-5-amine hydrochloride (Int-7-4) (3.8 g, yield 91%). MS(ESI): m/z 190.1 (M-NH₂).

### Step 4: Synthesis of tert-butyl (R)-4-(2-((1-nitro-6,7,8,9-tetrahydro-5H-benzo[7]annulen-5-yl)amino)acetamido)-2-((1-((2-(trimethylsilyl)ethoxy)methyl)-1H-pyrrolo[2,3-b]pyridin-5-yl)oxy)benzoate (Int-7-12)

To a solution of (R)-1-nitro-6,7,8,9-tetrahydro-5H-benzo[7]annulen-5-amine hydrochloride (Int-7-4) (1.5 g, 6.18 mmol, 1.0 eq), tert-butyl 4-(2-chloroacetamido)-2-((1-((2-(trimethylsilyl)ethoxy)methyl)-1H-pyrrolo[2,3-b]pyridin-5-yl)oxy)benzoate (Int-7-11) (3.3 g, 6.18 mmol, 1.0 eq), NaI (2.8 g, 18.54 mmol, 3.0 eq), K₂CO₃ (3.4 g, 24.72 mmol, 4.0 eq) in acetonitrile (50 mL) was heated at 90° C. for 5 h. H₂O (100 mL) was added and the mixture was extracted with EA (50 mL×3). The combined organic phase was washed with brine, dried over anhydrous Na₂SO₄ and concentrated under vacuum. The crude residue was purified by combine flash (EA/PE=0˜40%) to give tert-butyl (R)-4-(2-((1-nitro-6,7,8,9-tetrahydro-5H-benzo[7]annulen-5-yl)amino)acetamido)-2-((1-((2-(trimethylsilyl)ethoxy)methyl)-1H-pyrrolo[2,3-b]pyridin-5-yl)oxy)benzoate (Int-7-12) (4.2 g, yield 97%). MS(ESI): m/z 702.3 (M+H⁺).

### Step 5: Synthesis of tert-butyl (R)-4-(2-(2-chloro-N-(1-nitro-6,7,8,9-tetrahydro-5H-benzo[7]annulen-5-yl)acetamido)acetamido)-2-((1-((2-(trimethylsilyl)ethoxy)methyl)-1H-pyrrolo[2,3-b]pyridin-5-yl)oxy)benzoate (Int-7-13)

To a solution of tert-butyl (R)-4-(2-((1-nitro-6,7,8,9-tetrahydro-5H-benzo[7]annulen-5-yl)amino)acetamido)-2-((1-((2-(trimethylsilyl)ethoxy)methyl)-1H-pyrrolo[2,3-b]pyridin-5-yl)oxy)benzoate (Int-7-12) (4.2 g, 5.98 mmol, 1.0 eq) in DCM (50 mL) was added TEA (3.3 mL, 23.94 mmol, 4.0 eq) and 2-chloroacetyl chloride (1.35 g, 11.94 mmol, 2.0 eq) at 0° C. The reaction mixture was stirred at RT for 1 h. The organic phase was washed with saturated NaHCO₃ (30 mL×1), brine, dried over anhydrous Na₂SO₄ and concentrated under vacuum. The crude residue was purified by combine flash (EA/PE=0˜40%) to give tert-butyl (R)-4-(2-(2-chloro-N-(1-nitro-6,7,8,9-tetrahydro-5H-benzo[7]annulen-5-yl)acetamido)acetamido)-2-((1-((2-(trimethylsilyl)ethoxy)methyl)-1H-pyrrolo[2,3-b]pyridin-5-yl)oxy)benzoate (Int-7-13) (4.5 g, yield 97%). MS(ESI): m/z 778.3 (M+H⁺).

### Step 6: Synthesis of tert-butyl (R)-4-(4-(1-nitro-6,7,8,9-tetrahydro-5H-benzo[7]annulen-5-yl)-2,5-dioxopiperazin-1-yl)-2-((1-((2-(trimethylsilyl)ethoxy)methyl)-1H-pyrrolo[2,3-b]pyridin-5-yl)oxy)benzoate (Int-7-14)

To a solution of tert-butyl (R)-4-(2-(2-chloro-N-(1-nitro-6,7,8,9-tetrahydro-5H-benzo[7]annulen-5-yl)acetamido)acetamido)-2-((1-((2-(trimethylsilyl)ethoxy)methyl)-1H-pyrrolo[2,3-b]pyridin-5-yl)oxy)benzoate (Int-7-13) (5.1 g, 6.56 mmol, 1.0 eq), NaI (3.0 g, 19.67 mmol, 3.0 eq), K₂CO₃ (2.7 g, 19.67 mmol, 3.0 eq) in acetonitrile (50 mL) was heated at 90° C. for 5 h. EA (150 mL) was added and the organic phase was washed with H₂O (50 mL×1), brine, dried over anhydrous Na₂SO₄ and concentrated under vacuum. The crude residue was purified by combine flash (EA/PE=0˜40%) to give tert-butyl (R)-4-(4-(1-nitro-6,7,8,9-tetrahydro-5H-benzo[7]annulen-5-yl)-2,5-dioxopiperazin-1-yl)-2-((1-((2-(trimethylsilyl)ethoxy)methyl)-1H-pyrrolo[2,3-b]pyridin-5-yl)oxy)benzoate (Int-7-14) (3.8 g, yield 78%). MS(ESI): m/z 742.3 (M+H⁺).

### Step 7: Synthesis of tert-butyl (R)-4-(4-(1-nitro-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-7-15)

To a solution of tert-butyl (R)-4-(4-(1-nitro-6,7,8,9-tetrahydro-5H-benzo[7]annulen-5-yl)-2,5-dioxopiperazin-1-yl)-2-((1-((2-(trimethylsilyl)ethoxy)methyl)-1H-pyrrolo[2,3-b]pyridin-5-yl)oxy)benzoate (Int-7-14) (2.9 g, 3.91 mmol, 1.0 eq) in THE (40 mL) was added 1M BH₃·THF (39 mL, 39.08 mmol, 10.0 eq) and the mixture was stirred at RT for 2 h. MeOH (50 mL) was added slowly and the mixture was stirred at 60° C. overnight. The solvent was removed under vacuum. H₂O (80 mL) was added and the mixture was extracted with EA (50 mL×3). The combined organic phase was wished with brine, dried over anhydrous Na₂SO₄ and concentrated under vacuum. The crude residue was purified by combine flash (EA/PE=0˜40%) to give tert-butyl (R)-4-(4-(1-nitro-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-7-15) (1.9 g, yield 68%). MS(ESI): m/z 714.3 (M+H⁺).

### Step 8: Synthesis of (R)-2-((1H-pyrrolo[2,3-b]pyridin-5-yl)oxy)-4-(4-(1-nitro-6,7,8,9-tetrahydro-5H-benzo[7]annulen-5-yl)piperazin-1-yl)benzoic acid (Int-7-16)

To a solution of give tert-butyl (R)-4-(4-(1-nitro-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-7-15) (1.9 g, 3.91 mmol) in DCM (20 mL) was added TFA (20 mL). The reaction mixture was stirred at rt for 2 h. LC-MS monitored and the starting material was consumed completely. The mixture was concentrated under vacuum. The residue was dissolved in MeOH (30 mL) and added K₂CO₃ aqueous to adjust pH>8. The mixture was stirred at RT overnight. 1M HCl aqueous solution was added to adjust the pH to 5 and methanol was removed under vacuum. The mixture was extracted with DCM (60 mL×3). The combined organic phase was washed with brine, dried over anhydrous Na₂SO₄ and concentrated to give (R)-2-((1H-pyrrolo[2,3-b]pyridin-5-yl)oxy)-4-(4-(1-nitro-6,7,8,9-tetrahydro-5H-benzo[7]annulen-5-yl)piperazin-1-yl)benzoic acid (Int-7-16) (1.3 g, yield 93%). The crude product was used in the next step directly. MS(ESI): m/z 528.2 (M+H⁺).

### Step 9: Synthesis of (R)-2-((1H-pyrrolo[2,3-b]pyridin-5-yl)oxy)-N-((3-nitro-4-(((tetrahydro-2H-pyran-4-yl)methyl)amino)phenyl)sulfonyl)-4-(4-(1-nitro-6,7,8,9-tetrahydro-5H-benzo[7]annulen-5-yl)piperazin-1-yl)benzamide (Compound 73)

To a solution of (R)-2-((1H-pyrrolo[2,3-b]pyridin-5-yl)oxy)-4-(4-(1-nitro-6,7,8,9-tetrahydro-5H-benzo[7]annulen-5-yl)piperazin-1-yl)benzoic acid (Int7-16) (150 mg, 0.28 mmol, 1.0 eq) and 3-nitro-4-(((tetrahydro-2H-pyran-4-yl)methyl)amino)benzenesulfonamide (Int-7-17) (89 mg, 0.28 mmol, 1.0 eq) in anhydrous DMF (3 mL) was added EDCI (69 mg, 0.36 mmol, 1.3 eq), DMAP (159 mg, 0.84 mmol, 3.0 eq) and DIEA (108 mg, 0.84 mmol, 3.0 eq) at RT. The reaction mixture was stirred at RT for 16 h under N₂ atmosphere. LCMS showed the reaction was completed. The reaction mixture was poured into EtOAc (30 mL) and washed with H₂O (2×25 mL). The extract was washed with brine (1×20 mL), dried over Na₂SO₄. The crude product was purified by Prep-HPLC to give (R)-2-((1H-pyrrolo[2,3-b]pyridin-5-yl)oxy)-N-((3-nitro-4-(((tetrahydro-2H-pyran-4-yl)methyl)amino)phenyl)sulfonyl)-4-(4-(1-nitro-6,7,8,9-tetrahydro-5H-benzo[7]annulen-5-yl)piperazin-1-yl)benzamide (Compound 73) (40.7 mg, yield 17%). MS(ESI): m/z 825.3 (M+H⁺).

The following compounds were prepared according to the above described methods using different starting materials.

| Compound | | MS(ESI): m/z |
|---|---|---|
| No. | IUPAC Name | (M + H⁺) |
| 31A | (R)-2-((1H-pyrrolo[2,3-b]pyridin-5-yl)oxy)-4-(4-(1- | 829.1 |
| | chloro-6,7,8,9-tetrahydro-5H-benzo[7]annulen-5- | |
| | yl)piperazin-1-yl)-N-((4-((2- | |
| | morpholinoethyl)amino)-3- | |
| | nitrophenyl)sulfonyl)benzamide | |
| 70 | (R)-2-((1H-pyrrolo[2,3-b]pyridin-5-yl)oxy)-4-(4-(1- | 814.3 |
| | chloro-6,7,8,9-tetrahydro-5H-benzo[7]annulen-5- | |
| | yl)piperazin-1-yl)-N-((3-nitro-4-(((tetrahydro-2H- | |
| | pyran-4- | |
| | yl)methyl)amino)phenyl)sulfonyl)benzamide | |
| 71 | (R)-2-((1H-pyrrolo[2,3-b]pyridin-5-yl)oxy)-N-((4- | 853.9 |
| | (((2-oxaspiro[3.5]nonan-7-yl)methyl)amino)-3- | |
| | nitrophenyl)sulfonyl)-4-(4-(1-chloro-6,7,8,9- | |
| | tetrahydro-5H-benzo[7]annulen-5-yl)piperazin-1- | |
| | yl)benzamide | |
| 72 | (R)-2-((1H-pyrrolo[2,3-b]pyridin-5-yl)oxy)-N-((4- | 826.3 |
| | (((2-oxaspiro[3.3]heptan-6-yl)methyl)amino)-3- | |
| | nitrophenyl)sulfonyl)-4-(4-(1-chloro-6,7,8,9- | |
| | tetrahydro-5H-benzo[7]annulen-5-yl)piperazin-1- | |
| | yl)benzamide | |
| 74 | (R)-2-((1H-pyrrolo[2,3-b]pyridin-5-yl)oxy)-N-((4- | 865.3 |
| | (((2-oxaspiro[3.5]nonan-7-yl)methyl)amino)-3- | |
| | nitrophenyl)sulfonyl)-4-(4-(1-nitro-6,7,8,9- | |
| | tetrahydro-5H-benzo[7]annulen-5-yl)piperazin-1- | |
| | yl)benzamide | |
| 75 | (R)-2-((1H-pyrrolo[2,3-b]pyridin-5-yl)oxy)-N-((4- | 837.3 |
| | (((2-oxaspiro[3.3]heptan-6-yl)methyl)amino)-3- | |
| | nitrophenyl)sulfonyl)-4-(4-(1-nitro-6,7,8,9- | |
| | tetrahydro-5H-benzo[7]annulen-5-yl)piperazin-1- | |
| | yl)benzamide | |
| 77 | (R)-2-((1H-pyrrolo[2,3-b]pyridin-5-yl)oxy)-N-((4- | 870.3 |
| | (((2-oxaspiro[3.3]heptan-6-yl)methyl)amino)-3- | |
| | nitrophenyl)sulfonyl)-4-(4-(1-bromo-6,7,8,9- | |
| | tetrahydro-5H-benzo[7]annulen-5-yl)piperazin-1- | |
| | yl)benzamide | |
| 78 | (R)-2-((1H-pyrrolo[2,3-b]pyridin-5-yl)oxy)-4-(4-(1- | 876.2 |
| | bromo-3-fluoro-6,7,8,9-tetrahydro-5H- | |
| | benzo[7]annulen-5-yl)piperazin-1-yl)-N-((3-nitro-4- | |
| | (((tetrahydro-2H-pyran-4- | |
| | yl)methyl)amino)phenyl)sulfonyl)benzamide | |
| 79 | (R)-2-((1H-pyrrolo[2,3-b]pyridin-5-yl)oxy)-N-((4- | 916.2 |
| | (((2-oxaspiro[3.5]nonan-7-yl)methyl)amino)-3- | |
| | nitrophenyl)sulfonyl)-4-(4-(1-bromo-3-fluoro- | |
| | 6,7,8,9-tetrahydro-5H-benzo[7]annulen-5- | |
| | yl)piperazin-1-yl)benzamide | |
| 80 | (R)-2-((1H-pyrrolo[2,3-b]pyridin-5-yl)oxy)-N-((4- | 931.2 |
| | ((2-(2-oxa-7-azaspiro[3.5]nonan-7-yl)ethyl)amino)- | |
| | 3-nitrophenyl)sulfonyl)-4-(4-(1-bromo-3-fluoro- | |
| | 6,7,8,9-tetrahydro-5H-benzo[7]annulen-5- | |
| | yl)piperazin-1-yl)benzamide | |
| 81 | 2-((1H-pyrrolo[2,3-b]pyridin-5-yl)oxy)-4-(4-((R)-1- | 904.2 |
| | bromo-3-fluoro-6,7,8,9-tetrahydro-5H- | |
| | benzo[7]annulen-5-yl)piperazin-1-yl)-N-((4- | |
| | ((((3R,3aR,6aS)-hexahydrofuro[2,3-b]furan-3- | |
| | yl)methyl)amino)-3- | |
| | nitrophenyl)sulfonyl)benzamide | |
| 82 | 2-((1H-pyrrolo[2,3-b]pyridin-5-yl)oxy)-4-(4-((R)-1- | 904.2 |
| | bromo-3-fluoro-6,7,8,9-tetrahydro-5H- | |
| | benzo[7]annulen-5-yl)piperazin-1-yl)-N-((4- | |
| | ((((3S,3aS,6aR)-hexahydrofuro[2,3-b]furan-3- | |
| | yl)methyl)amino)-3- | |
| | nitrophenyl)sulfonyl)benzamide | |
| 83 | (R)-2-((1H-pyrrolo[2,3-b]pyridin-5-yl)oxy)-4-(4-(1- | 869.3 |
| | chloro-6,7,8,9-tetrahydro-5H-benzo[7]annulen-5- | |
| | yl)piperazin-1-yl)-N-((3-nitro-4-(((1-(oxetan-3- | |
| | yl)piperidin-4- | |
| | yl)methyl)amino)phenyl)sulfonyl)benzamide | |
| 84 | (R)-2-((1H-pyrrolo[2,3-b]pyridin-5-yl)oxy)-4-(4-(1- | 884.2 |
| | chloro-6,7,8,9-tetrahydro-5H-benzo[7]annulen-5- | |
| | yl)piperazin-1-yl)-N-((3-nitro-4-((2-(4-(oxetan-3- | |
| | yl)piperazin-1- | |
| | yl)ethyl)amino)phenyl)sulfonyl)benzamide | |
| 85 | (R)-2-((1H-pyrrolo[2,3-b]pyridin-5-yl)oxy)-4-(4-(1- | 885.3 |
| | chloro-6,7,8,9-tetrahydro-5H-benzo[7]annulen-5- | |
| | yl)piperazin-1-yl)-N-((4-(((4-hydroxy-1-(oxetan-3- | |
| | yl)piperidin-4-yl)methyl)amino)-3- | |
| | nitrophenyl)sulfonyl)benzamide | |
| 86 | (R)-2-((1H-pyrrolo[2,3-b]pyridin-5-yl)oxy)-4-(4-(1- | 845.3 |
| | chloro-3-fluoro-6,7,8,9-tetrahydro-5H- | |
| | benzo[7]annulen-5-yl)piperazin-1-yl)-N-((4-(((1- | |
| | methylpiperidin-4-yl)methyl)amino)-3- | |
| | nitrophenyl)sulfonyl)benzamide | |
| 87 | (R)-2-((1H-pyrrolo[2,3-b]pyridin-5-yl)oxy)-4-(4-(1- | 832.3 |
| | chloro-3-fluoro-6,7,8,9-tetrahydro-5H- | |
| | benzo[7]annulen-5-yl)piperazin-1-yl)-N-((3-nitro-4- | |
| | (((tetrahydro-2H-pyran-4- | |
| | yl)methyl)amino)phenyl)sulfonyl)benzamide | |
| 88 | (R)-2-((1H-pyrrolo[2,3-b]pyridin-5-yl)oxy)-4-(4-(1- | 903.2 |
| | chloro-3-fluoro-6,7,8,9-tetrahydro-5H- | |
| | benzo[7]annulen-5-yl)piperazin-1-yl)-N-((4-(((4- | |
| | hydroxy-1-(oxetan-3-yl)piperidin-4- | |
| | yl)methyl)amino)-3- | |
| | nitrophenyl)sulfonyl)benzamide | |
| 89 | (R)-2-((1H-pyrrolo[2,3-b]pyridin-5-yl)oxy)-4-(4-(1- | 798.3 |
| | fluoro-6,7,8,9-tetrahydro-5H-benzo[7]annulen-5- | |
| | yl)piperazin-1-yl)-N-((3-nitro-4-(((tetrahydro-2H- | |
| | pyran-4- | |
| | yl)methyl)amino)phenyl)sulfonyl)benzamide | |
| 99 | (R)-2-((1H-pyrrolo[2,3-b]pyridin-5-yl)oxy)-N-((4- | 854.3 |
| | (((7-oxaspiro[3.5]nonan-2-yl)methyl)amino)-3- | |
| | nitrophenyl)sulfonyl)-4-(4-(1-chloro-6,7,8,9- | |
| | tetrahydro-5H-benzo[7]annulen-5-yl)piperazin-1- | |
| | yl)benzamide | |
| 100 | tert-butyl (R)-2-(((4-(N-(2-((1H-pyrrolo[2,3- | 953.3 |
| | b]pyridin-5-yl)oxy)-4-(4-(1-chloro-6,7,8,9- | |
| | tetrahydro-5H-benzo[7]annulen-5-yl)piperazin-1- | |
| | yl)benzoyl)sulfamoyl)-2- | |
| | nitrophenyl)amino)methyl)-7-azaspiro[3.5]nonane- | |
| | 7-carboxylate | |
| 101 | 2-((1H-pyrrolo[2,3-b]pyridin-5-yl)oxy)-4-(4-((R)-1- | 842.2 |
| | chloro-6,7,8,9-tetrahydro-5H-benzo[7]annulen-5- | |
| | yl)piperazin-1-yl)-N-((4-(((3,3-dimethyltetrahydro- | |
| | 2H-pyran-4-yl)methyl)amino)-3- | |
| | nitrophenyl)sulfonyl)benzamide | |
| 102 | 2-((1H-pyrrolo[2,3-b]pyridin-5-yl)oxy)-4-(4-((R)-1- | 842.2 |
| | chloro-6,7,8,9-tetrahydro-5H-benzo[7]annulen-5- | |
| | yl)piperazin-1-yl)-N-((4-(((2,2-dimethyltetrahydro- | |
| | 2H-pyran-4-yl)methyl)amino)-3- | |
| | nitrophenyl)sulfonyl)benzamide | |
| 103 | tert-butyl 5-(((4-(N-(2-((1H-pyrrolo[2,3-b]pyridin- | 925.3 |
| | 5-yl)oxy)-4-(4-((R)-1-chloro-6,7,8,9-tetrahydro-5H- | |
| | benzo[7]annulen-5-yl)piperazin-1- | |
| | yl)benzoyl)sulfamoyl)-2- | |
| | nitrophenyl)amino)methyl)-2- | |
| | azabicyclo[2.2.1]heptane-2-carboxylate | |
| 108 | (R)-2-((1H-pyrrolo[2,3-b]pyridin-5-yl)oxy)-4-(4-(1- | 867.2 |
| | chloro-6,7,8,9-tetrahydro-5H-benzo[7]annulen-5- | |
| | yl)piperazin-1-yl)-N-((4-(((7-methyl-7- | |
| | azaspiro[3.5]nonan-2-yl)methyl)amino)-3- | |
| | nitrophenyl)sulfonyl)benzamide | |
| 109 | tert-butyl (R)-(2-(2-(((4-(N-(2-((1H-pyrrolo[2,3- | 996.2 |
| | b]pyridin-5-yl)oxy)-4-(4-(1-chloro-6,7,8,9- | |
| | tetrahydro-5H-benzo[7]annulen-5-yl)piperazin-1- | |
| | yl)benzoyl)sulfamoyl)-2- | |
| | nitrophenyl)amino)methyl)-7-azaspiro[3.5]nonan-7- | |
| | yl)ethyl)carbamate | |
| 111 | (R)-2-((1H-pyrrolo[2,3-b]pyridin-5-yl)oxy)-N-((4- | 895.2 |
| | (((7-acetyl-7-azaspiro[3.5]nonan-2- | |
| | yl)methyl)amino)-3-nitrophenyl)sulfonyl)-4-(4-(1- | |
| | chloro-6,7,8,9-tetrahydro-5H-benzo[7]annulen-5- | |
| | yl)piperazin-1-yl)benzamide | |
| 112 | (R)-2-((1H-pyrrolo[2,3-b]pyridin-5-yl)oxy)-N-((4- | 938.3 |
| | (((7-(2-acetamidoethyl)-7-azaspiro[3.5]nonan-2- | |
| | yl)methyl)amino)-3-nitrophenyl)sulfonyl)-4-(4-(1- | |
| | chloro-6,7,8,9-tetrahydro-5H-benzo[7]annulen-5- | |
| | yl)piperazin-1-yl)benzamide | |
| 113 | (R)-2-((1H-pyrrolo[2,3-b]pyridin-5-yl)oxy)-4-(4-(1- | 917.1 |
| | chloro-6,7,8,9-tetrahydro-5H-benzo[7]annulen-5- | |
| | yl)piperazin-1-yl)-N-((4-(((7-(2,2-difluoroethyl)-7- | |
| | azaspiro[3.5]nonan-2-yl)methyl)amino)-3- | |
| | nitrophenyl)sulfonyl)benzamide | |
| 114 | (R)-2-((1H-pyrrolo[2,3-b]pyridin-5-yl)oxy)-4-(4-(1- | 899.5 |
| | chloro-6,7,8,9-tetrahydro-5H-benzo[7]annulen-5- | |
| | yl)piperazin-1-yl)-N-((4-(((7-(2-fluoroethyl)-7- | |
| | azaspiro[3.5]nonan-2-yl)methyl)amino)-3- | |
| | nitrophenyl)sulfonyl)benzamide | |
| 115 | (R)-2-((1H-pyrrolo[2,3-b]pyridin-5-yl)oxy)-4-(4-(1- | 935.2 |
| | chloro-6,7,8,9-tetrahydro-5H-benzo[7]annulen-5- | |
| | yl)piperazin-1-yl)-N-((3-nitro-4-(((7-(2,2,2- | |
| | trifluoroethyl)-7-azaspiro[3.5]nonan-2- | |
| | yl)methyl)amino)phenyl)sulfonyl)benzamide | |

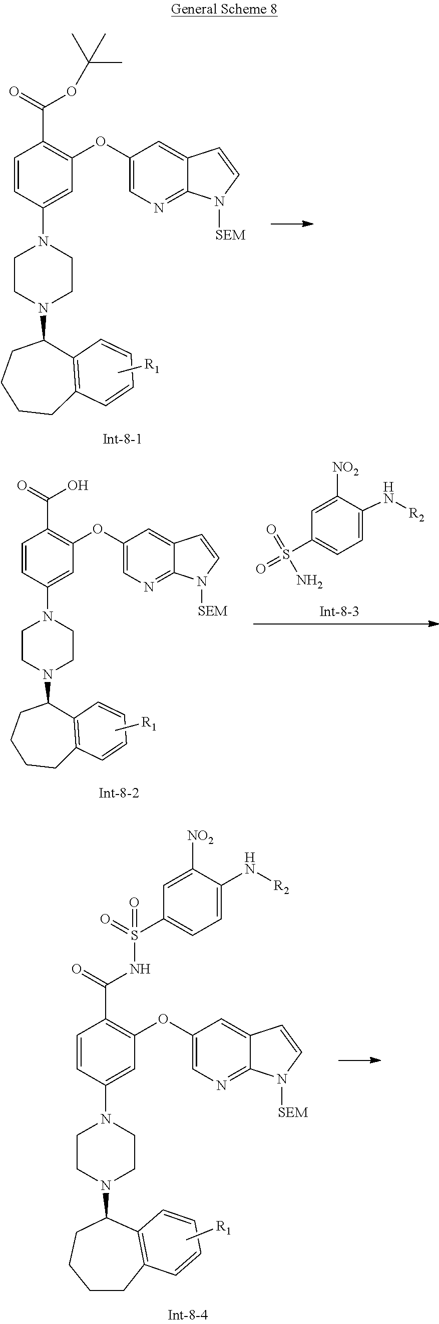

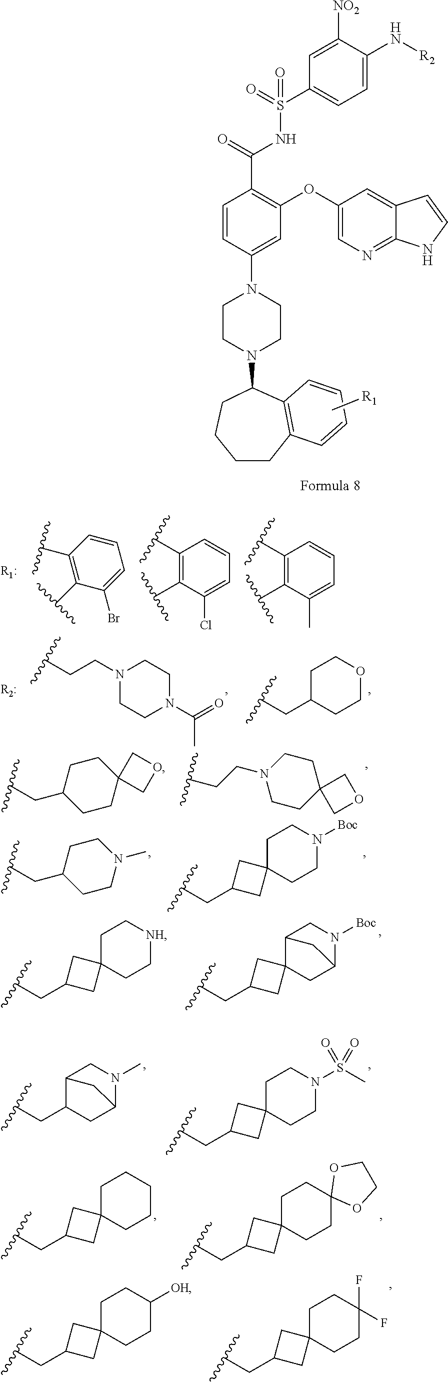

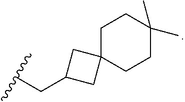 [SMILES: *CC1CC2(CCC(C)(C)CC2)C1]

### General Step 1: Synthesis of Int-8-2

To a solution of Int-8-1 (10.0 mmol, 1.0 eq) in EtOH (100 mL) was added 2M NaOH aqueous solution (50 mL, 100.0 mmol, 10 eq). The reaction mixture was stirred at 80° C. overnight. Ethanol was removed under vacuum. HCl aqueous solution was added to adjust pH to 6 and it was extracted with DCM (3×100 mL). The combined organic phase was washed with brine (1×50 mL), dried over sodium sulfate and concentrated under reduced pressure. The residue was purified by flash chromatography (ME/DCM=0˜10%) to give Int-8-2.

### General Step 2: Synthesis of Int-8-4

To a solution of Int-8-2 (1.0 mmol, 1.0 eq) and Int-8-3 (1.0 mmol, 1.0 eq) in DMF (5 mL) was added DIPEA (3.0 mmol, 3.0 eq), DMAP (3.0 mmol, 3.0 eq) and EDCI (1.3 mmol, 1.3 eq). The reaction mixture was stirred at rt overnight. EA (50 mL) was added and the mixture was washed with water (20 mL×3), brine, dried over anhydrous Na₂SO₄ and concentrated. The residue was purified by flash chromatography (ME/DCM=0-10%) to give Int-8-4.

### General Step 3: Synthesis of Formula 8

To a solution of Int-8-4 (0.27 mmol) in DCM (4 mL) was added TFA (2 mL). The reaction mixture was stirred at rt for 2 h. LC-MS monitored and the starting material was consumed completely. The mixture was concentrated under vacuum. The residue was dissolved in MeOH (8 mL) and DCM (4 mL). K₂CO₃ aqueous solution was added to adjust pH>8. The mixture was stirred at RT overnight. 1M HCl aqueous solution was added to adjust the pH to 7 and the mixture was extracted with DCM (20 mL×3). The combined organic phase was washed with brine, dried over anhydrous Na₂SO₄ and concentrated. The crude mixture was purified by Prep-HPLC to give Formula 8.

### Example 14: Synthesis of Compound 68

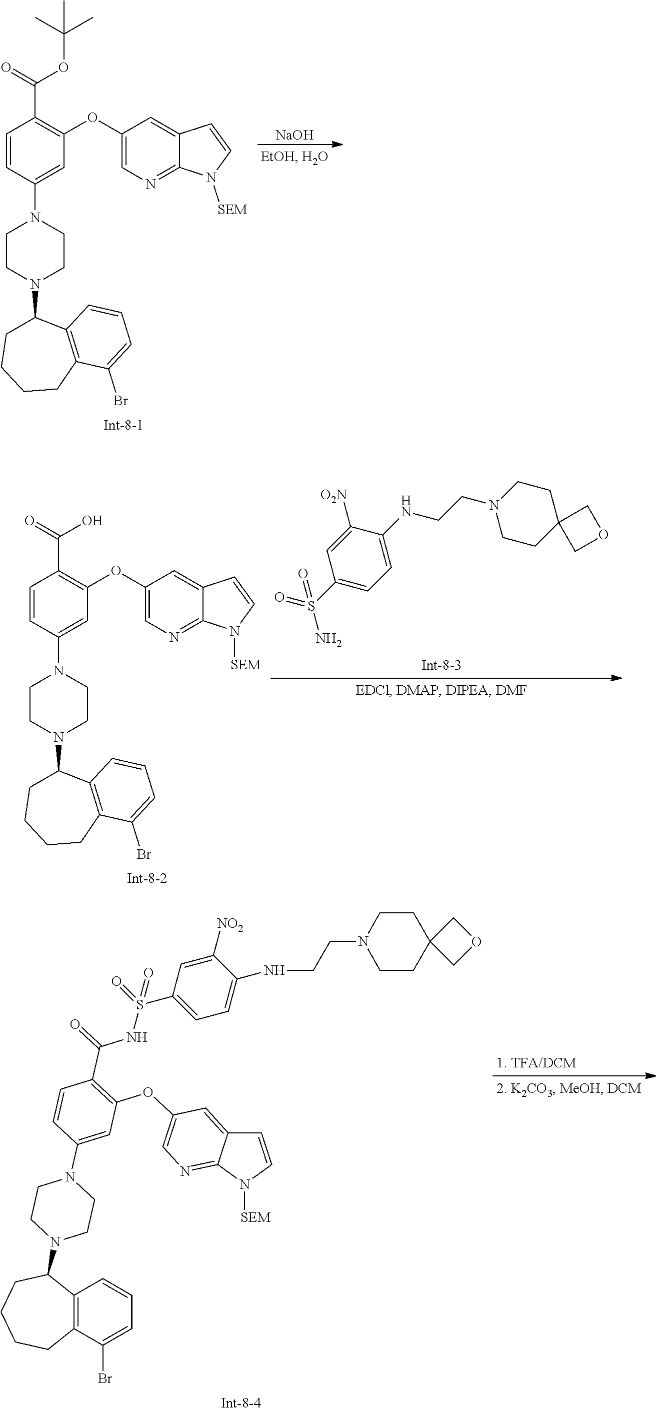

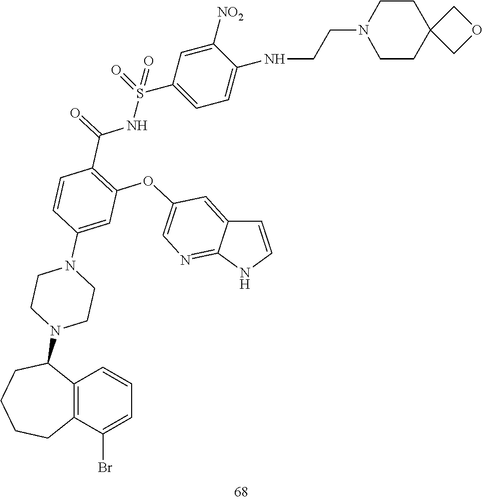 [SMILES: O=C(NS(=O)(=O)c1ccc(NCCN2CCC3(CC2)COC3)c([N+](=O)[O-])c1)c1ccc(N2CCN([C@@H]3CCCCc4c(Br)cccc43)CC2)cc1Oc1cnc2[nH]ccc2c1]

### Step 1: Synthesis of (R)-4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoic acid (Int-8-2)

To a solution of tert-butyl (R)-4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-8-1) (2.0 g, 2.68 mmol, 1.0 eq) in EtOH (30 mL) was added 2M NaOH aqueous solution (13.5 mL, 27.0 mmol, 10 eq). The reaction mixture was stirred at 80° C. overnight. Ethanol was removed under vacuum. HCl aqueous solution was added to adjust pH to 6 and it was extracted with DCM (3×100 mL). The combined organic phase was washed with brine (1×50 mL), dried over sodium sulfate and concentrated under reduced pressure. The residue was purified by flash chromatography (ME/DCM=0-10%) to give (R)-4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoic acid (Int-8-2) (1.5 g, 81%). MS(ESI): m/z 691.2 (M+H⁺).

### Step 2: Synthesis of (R)—N-((4-((2-(2-oxa-7-azaspiro[3.5]nonan-7-yl)ethyl)amino)-3-nitrophenyl)sulfonyl)-4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzamide (Int-8-4)

To a stirred solution of (R)-4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoic acid (Int-8-2) (200 mg, 0.29 mmol, 1.0 eq) in anhydrous DMF (3 ml) was added 4-((2-(2-oxa-7-azaspiro[3.5]nonan-7-yl)ethyl)amino)-3-nitrobenzenesulfonamide (Int-8-3) (107 mg, 0.29 mmol, 1.0 eq), EDCI (72 mg, 0.38 mmol, 1.3 eq), DMAP (106 mg, 0.87 mmol, 3.0 eq), DIPEA (112 mg, 0.87 mmol, 3.0 eq). The mixture was stirred at RT for 16 h. The reaction mixture was concentrated, and the residue was dissolved in EA (50 mL). The organic layer was washed with water (30 mL×3), dried over anhydrous Na₂SO₄ and evaporated in vacuum. The residue was purified by flash chromatography (ME/DCM=0-10%) to give (R)—N-((4-((2-(2-oxa-7-azaspiro[3.5]nonan-7-yl)ethyl)amino)-3-nitrophenyl)sulfonyl)-4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzamide (Int-8-4) (150 mg, yield 50%). MS(ESI): m/z 1043.3 (M+H⁺).

### Step 3: Synthesis of (R)-2-((1H-pyrrolo[2,3-b]pyridin-5-yl)oxy)-N-((4-((2-(2-oxa-7-azaspiro[3.5]nonan-7-yl)ethyl)amino)-3-nitrophenyl)sulfonyl)-4-(4-(1-bromo-6,7,8,9-tetrahydro-5H-benzo[7]annulen-5-yl)piperazin-1-yl)benzamide (Compound 68)

To a solution of (R)—N-((4-((2-(2-oxa-7-azaspiro[3.5]nonan-7-yl)ethyl)amino)-3-nitrophenyl)sulfonyl)-4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzamide (Int-8-4) (150 mg, 0.14 mmol) in CH₂Cl₂ (4 mL) was added TFA (2 mL). The mixture was stirred at room temperature for 2 h. The solvent was removed under vacuum. The residue was dissolved in MeOH (8 mL) and DCM (4 mL). K₂CO₃ aqueous solution was added to adjust pH>8. The mixture was stirred at rt overnight. 1M HCl aqueous solution was added to adjust the pH to 7 and the mixture was extracted with DCM (20 mL×3). The combined organic phase was washed with brine, dried over anhydrous Na₂SO₄ and concentrated. The crude mixture was purified by Prep-HPLC to give (R)-2-((1H-pyrrolo[2,3-b]pyridin-5-yl)oxy)-N-((4-((2-(2-oxa-7-azaspiro[3.5]nonan-7-yl)ethyl)amino)-3-nitrophenyl)sulfonyl)-4-(4-(1-bromo-6,7,8,9-tetrahydro-5H-benzo[7]annulen-5-yl)piperazin-1-yl)benzamide (Compound 68) (50 mg, yield 38%). MS(ESI): m/z 913.2 (M+H⁺).

### Example 15: Synthesis of Compound 106

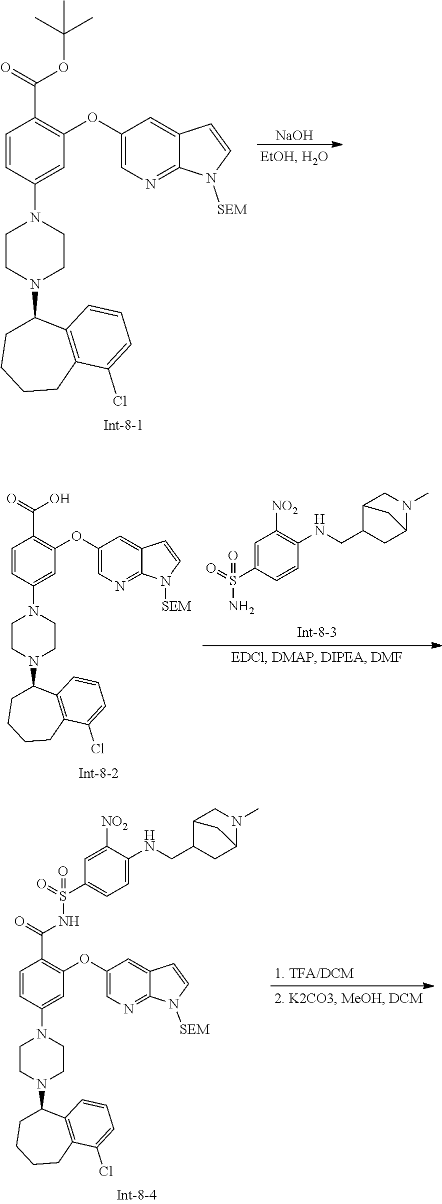

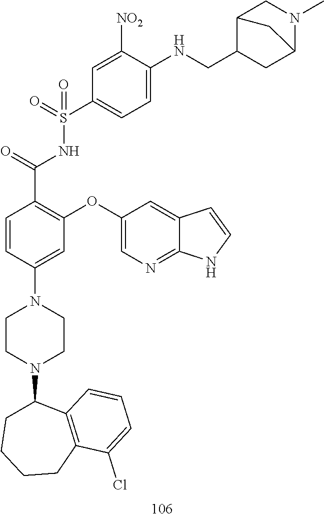 [SMILES: CN1CC2CC1CC2CNc1ccc(S(=O)(=O)NC(=O)c2ccc(N3CCN([C@@H]4CCCCc5c(Cl)cccc54)CC3)cc2Oc2cnc3[nH]ccc3c2)cc1[N+](=O)[O-]]

### Step 1: Synthesis of tert-butyl (R)-4-(4-(1-chloro-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-8-1)

Refer to the preparation of Int-7-15 in General Scheme 7. MS(ESI): m/z 703.2 (M+H⁺).

### Step 2: Synthesis of (R)-4-(4-(1-chloro-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoic acid (Int-8-2)

To a solution of tert-butyl (R)-4-(4-(1-chloro-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-8-1) (2.0 g, 2.84 mmol, 1.0 eq) in EtOH (30 mL) was added 2M NaOH aqueous solution (13.5 mL, 27.0 mmol, 10 eq). The reaction mixture was stirred at 80° C. overnight. Ethanol was removed under vacuum. HCl aqueous solution was added to adjust pH to 6 and it was extracted with DCM (3×100 mL). The combined organic phase was washed with brine (1×50 mL), dried over sodium sulfate and concentrated under (R)-4-(4-(1-chloro-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoic acid (Int-8-2) (1.5 g, 82%). MS(ESI): m/z 647.2 (M+H⁺).

### Step 3: Synthesis of 4-(4-((R)-1-chloro-6,7,8,9-tetrahydro-5H-benzo[7]annulen-5-yl)piperazin-1-yl)-N-((4-(((2-methyl-2-azabicyclo[2.2.1]heptan-5-yl)methyl)amino)-3-nitrophenyl)sulfonyl)-2-((1-((2-(trimethylsilyl)ethoxy)methyl)-1H-pyrrolo[2,3-b]pyridin-5-yl)oxy)benzamide (Int-8-4)

To a stirred solution of (R)-4-(4-(1-chloro-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoic acid (Int-8-2) (50 mg, 0.077 mmol, 1.0 eq) in anhydrous DMF (2 ml) was added 4-(((2-methyl-2-azabicyclo[2.2.1]heptan-5-yl)methyl)amino)-3-nitrobenzenesulfonamide (Int-8-3) (26 mg, 0.077 mmol, 1.0 eq), EDCI (19 mg, 0.10 mmol, 1.3 eq), DMAP (28 mg, 0.23 mmol, 3.0 eq), DIPEA (30 mg, 0.87 mmol, 3.0 eq). The mixture was stirred at rt for 16 h. The reaction mixture was concentrated, and the residue was dissolved in EA (50 mL). The organic layer was washed with water (30 mL×3), dried over anhydrous Na₂SO₄ and evaporated in vacuum. The residue was purified by flash chromatography (ME/DCM=0-10%) to give 4-(4-((R)-1-chloro-6,7,8,9-tetrahydro-5H-benzo[7]annulen-5-yl)piperazin-1-yl)-N-((4-(((2-methyl-2-azabicyclo[2.2.1]heptan-5-yl)methyl)amino)-3-nitrophenyl)sulfonyl)-2-((1-((2-(trimethylsilyl)ethoxy)methyl)-1H-pyrrolo[2,3-b]pyridin-5-yl)oxy)benzamide (Int-8-4) (30 mg, yield 40%). MS(ESI): m/z 969.3 (M+H⁺).

### Step 4: Synthesis of 2-((1H-pyrrolo[2,3-b]pyridin-5-yl)oxy)-4-(4-((R)-1-chloro-6,7,8,9-tetrahydro-5H-benzo[7]annulen-5-yl)piperazin-1-yl)-N-((4-(((2-methyl-2-azabicyclo[2.2.1]heptan-5-yl)methyl)amino)-3-nitrophenyl)sulfonyl)benzamide (Compound 106)

To a solution of 4-(4-((R)-1-chloro-6,7,8,9-tetrahydro-5H-benzo[7]annulen-5-yl)piperazin-1-yl)-N-((4-(((2-methyl-2-azabicyclo[2.2.1]heptan-5-yl)methyl)amino)-3-nitrophenyl)sulfonyl)-2-((1-((2-(trimethylsilyl)ethoxy)methyl)-1H-pyrrolo[2,3-b]pyridin-5-yl)oxy)benzamide (Int-8-4) (30 mg, 0.031 mmol) in CH2Cl2 (2 mL) was added TFA (1 mL). The mixture was stirred at room temperature for 2 h. The solvent was removed under vacuum. The residue was dissolved in MeOH (8 mL) and DCM (4 mL). K₂CO₃ aqueous solution was added to adjust pH>8. The mixture was stirred at rt overnight. 1M HCl aqueous solution was added to adjust the pH to 7 and the mixture was extracted with DCM (20 mL×3). The combined organic phase was washed with brine, dried over anhydrous Na₂SO₄ and concentrated. The crude mixture was purified by prep-HPLC to give 2-((1H-pyrrolo[2,3-b]pyridin-5-yl)oxy)-4-(4-((R)-1-chloro-6,7,8,9-tetrahydro-5H-benzo[7]annulen-5-yl)piperazin-1-yl)-N-((4-(((2-methyl-2-azabicyclo[2.2.1]heptan-5-yl)methyl)amino)-3-nitrophenyl)sulfonyl)benzamide (Compound 106) (8 mg, yield 31%). MS(ESI): m/z 839.3 (M+H⁺).

The following compounds were prepared according to the above described methods using different starting materials.

| Compound | | MS(ESI): m/z |
|---|---|---|
| No. | IUPAC Name | (M + H⁺) |
| 65 | (R)-2-((1H-pyrrolo[2,3-b]pyridin-5-yl)oxy)-N-((4- | 914.0 |
| | ((2-(4-acetylpiperazin-1-yl)ethyl)amino)-3- | |
| | nitrophenyl)sulfonyl)-4-(4-(1-bromo-6,7,8,9- | |
| | tetrahydro-5H-benzo[7]annulen-5-yl)piperazin-1- | |
| | yl)benzamide | |
| 67 | (R)-2-((1H-pyrrolo[2,3-b]pyridin-5-yl)oxy)-N-((4- | 897.9 |
| | (((2-oxaspiro[3.5]nonan-7-yl)methyl)amino)-3- | |
| | nitrophenyl)sulfonyl)-4-(4-(1-bromo-6,7,8,9- | |
| | tetrahydro-5H-benzo[7]annulen-5-yl)piperazin-1- | |
| | yl)benzamide | |
| 69 | (R)-2-((1H-pyrrolo[2,3-b]pyridin-5-yl)oxy)-4-(4-(1- | 871.2 |
| | bromo-6,7,8,9-tetrahydro-5H-benzo[7]annulen-5- | |
| | yl)piperazin-1-yl)-N-((4-(((1-methylpiperidin-4- | |
| | yl)methyl)amino)-3-nitrophenyl)sulfonyl)benzamide | |
| 90 | (R)-2-((1H-pyrrolo[2,3-b]pyridin-5-yl)oxy)-4-(4-(1- | 793.8 |
| | methyl-6,7,8,9-tetrahydro-5H-benzo[7]annulen-5- | |
| | yl)piperazin-1-yl)-N-((3-nitro-4-(((tetrahydro-2H- | |
| | pyran-4-yl)methyl)amino)phenyl)sulfonyl)benzamide | |
| 104 | (R)-2-((1H-pyrrolo[2,3-b]pyridin-5-yl)oxy)-N-((4- | 853.0 |
| | (((7-azaspiro[3.5]nonan-2-yl)methyl)amino)-3- | |
| | nitrophenyl)sulfonyl)-4-(4-(1-chloro-6,7,8,9- | |
| | tetrahydro-5H-benzo[7]annulen-5-yl)piperazin-1- | |
| | yl)benzamide | |
| 107 | (R)-2-((1H-pyrrolo[2,3-b]pyridin-5-yl)oxy)-4-(4-(1- | 931.2 |
| | chloro-6, 7,8,9-tetrahydro-5H-benzo[7]annulen-5- | |
| | yl)piperazin-1-yl)-N-((4-(((7-(methylsulfonyl)-7- | |
| | azaspiro[3.5]nonan-2-yl)methyl)amino)-3- | |
| | nitrophenyl)sulfonyl)benzamide | |

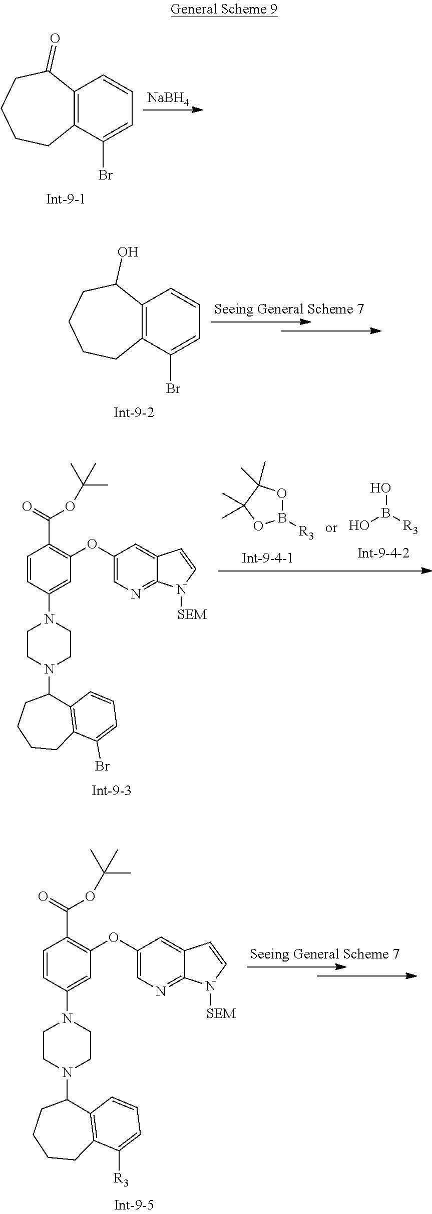

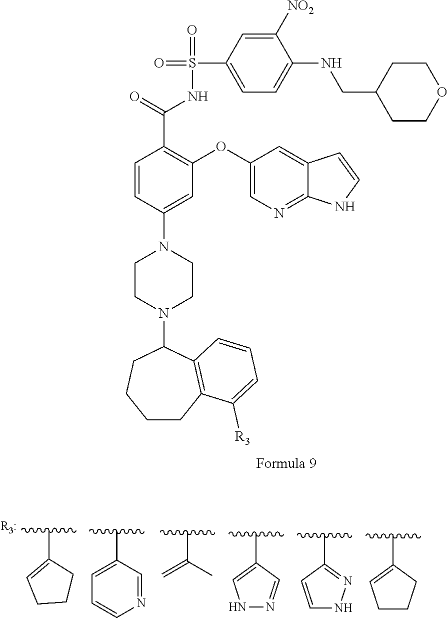

### General Step 1: Synthesis of Int-9-2

To a solution of the mixture of 1-bromo-6,7,8,9-tetrahydro-5H-benzo[7]annulen-5-one (Int-9-1) and 3-bromo-6,7,8,9-tetrahydro-5H-benzo[7]annulen-5-one (unseparated byproduct in the previous step) (28.0 g, 117 mmol, 1.0 eq) in methanol (300 mL) was added NaBH₄ (6.65 g, 176 mmol, 1.5 eq) at 0° C. and the reaction mixture was stirred at 0° C. for 2 h. H₂O (50 mL) was added to quench the reaction. The solvent was removed under vacuum. H₂O (100 mL) was added and the mixture was extracted with EA (100 mL×3). The combined organic phase was washed with brine, dried over anhydrous Na₂SO₄ and concentrated under vacuum. The crude residue was purified by combine flash (EA/PE=0˜30%) to give Int-9-2 (9.0 g, yield 32%). MS(ESI): m/z 224.2 (M-OH).

### General Step 2: Synthesis of Int-9-3

Seeing the preparation of Int-7-15 in General Scheme 7.

### General Step 3: Synthesis of Int-9-5

To a solution of Int-9-3 (0.27 mmol, 1.0 eq), Int-9-4 (0.80 mmol, 3.0 eq), Pd(PPh₂)Cl₂ (0.03 mmol, 0.1 eq), Cs₂CO₃ (0.53 mmol, 2.0 eq) in dioxane (5 mL) and H₂O (0.5 mL) was heated at 80° C. for 6 h. EA (30 mL) was added and the mixture was washed with H₂O (20 mL×1), brine, dried over anhydrous Na₂SO₄ and concentrated under vacuum. The crude residue was purified by combine flash (EA/PE=0˜40%) to give Int-9-5.

### General Step 4: Synthesis of Formula 9

Seeing the preparation of Formula 7 in General Scheme 7.

### Example 16: Synthesis of Compound 50

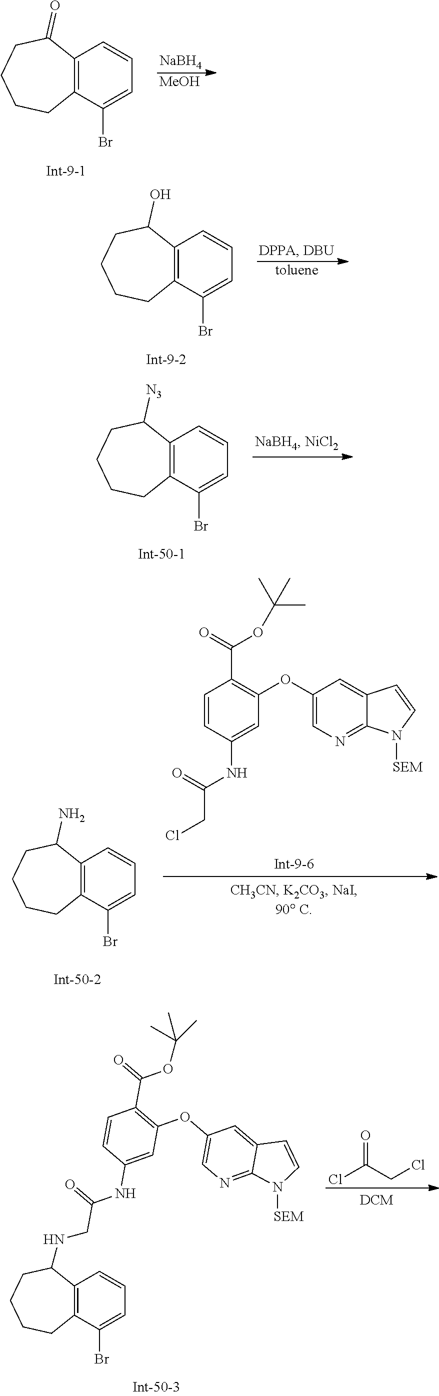

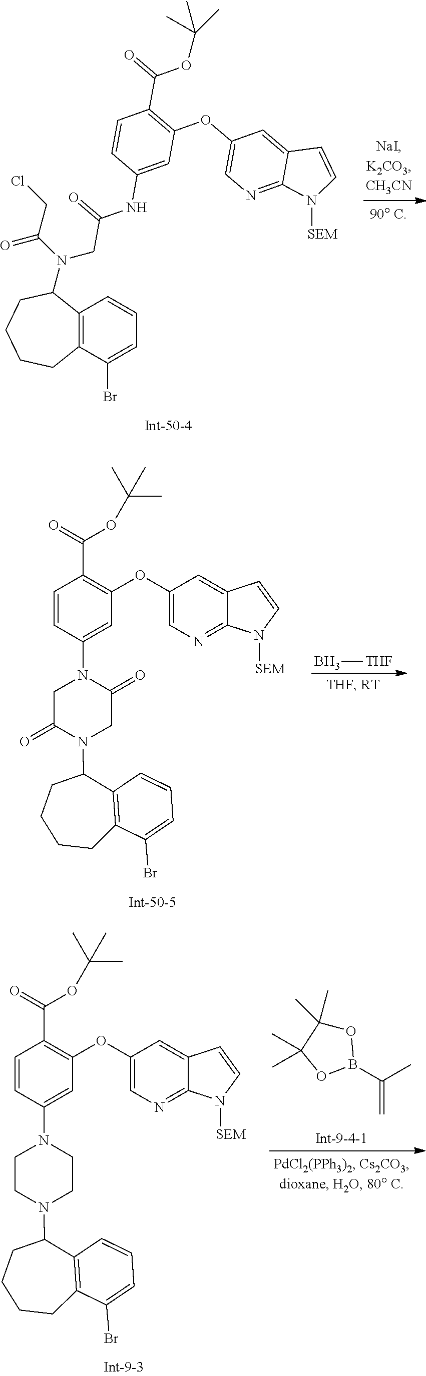

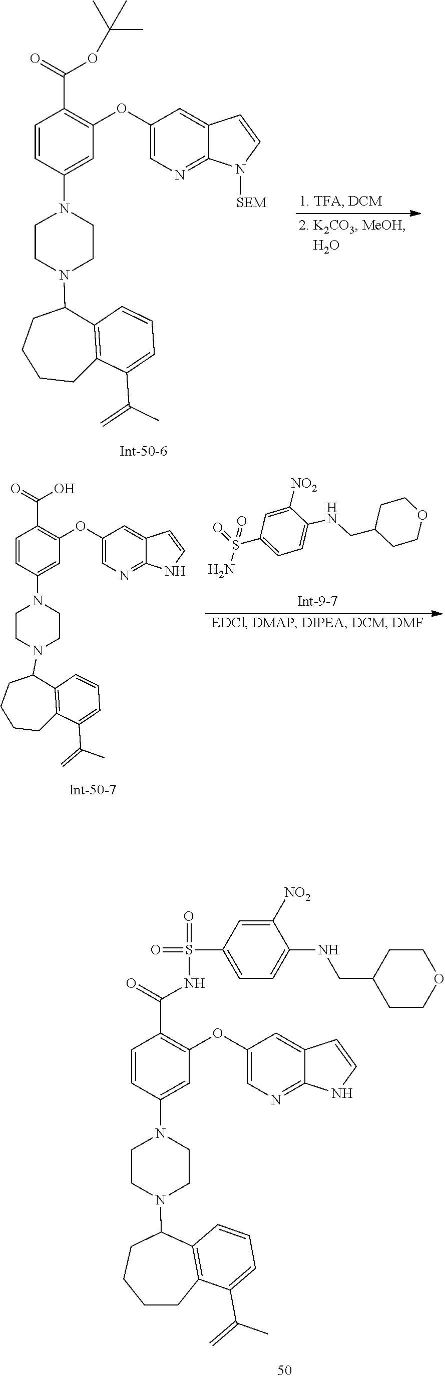

### Step 1: Synthesis of Int-9-2

To a solution of the mixture of 1-bromo-6,7,8,9-tetrahydro-5H-benzo[7]annulen-5-one (Int-9-1) and 3-bromo-6,7,8,9-tetrahydro-5H-benzo[7]annulen-5-one (unseparated byproduct in the previous step) (28.0 g, 117 mmol, 1.0 eq) in methanol (300 mL) was added NaBH₄ (6.65 g, 176 mmol, 1.5 eq) at 0° C. and the reaction mixture was stirred at 0° C. for 2 h. H₂O (50 mL) was added to quench the reaction. The solvent was removed under vacuum. H₂O (100 mL) was added and the mixture was extracted with EA (100 mL×3). The combined organic phase was washed with brine, dried over anhydrous Na₂SO₄ and concentrated under vacuum. The crude residue was purified by combine flash (EA/PE=0˜30%) to give 1-bromo-6,7,8,9-tetrahydro-5H-benzo[7]annulen-5-ol (Int-9-2) (9.0 g, yield 32%). MS(ESI): m/z 224.2 (M-OH).

### Step 2: Synthesis of Int-50-1

To a solution of compound 1-bromo-6,7,8,9-tetrahydro-5H-benzo[7]annulen-5-ol (Int-9-2) (2.3 g, 9.54 mmol, 1.0 eq) in toluene (20 mL) was added DPPA (4.1 mL, 19.08 mmol, 2.0 eq) and DBU (2.1 mL, 14.31 mmol, 1.5 eq). The reaction mixture was stirred under N₂ atmosphere at 50° C. for 4 hours. H₂O (50 mL) was added and the mixture was extracted with DCM (30 mL×3). The combined organic phase was washed with 1 M HCl aqueous solution (30 mL×1), brine, dried over anhydrous Na₂SO₄ and concentrated under vacuum. The crude residue was purified by combine flash (PE=100%) to give 5-azido-1-bromo-6,7,8,9-tetrahydro-5H-benzo[7]annulene (Int-50-1) (1.2 g, yield 47%). MS(ESI): m/z 224.2 (M-N3).

### Step 3: Synthesis of 1-bromo-6,7,8,9-tetrahydro-5H-benzo[7]annulen-5-amine (Int-50-2)

To a solution of 5-azido-1-bromo-6,7,8,9-tetrahydro-5H-benzo[7]annulene (Int-50-1) (1.2 g, 4.51 mmol, 1.0 eq) in methanol (20 mL) was added NiCl₂ (643 mg, 4.96 mmol, 1.1 eq) and NaBH₄ (256 mg, 6.76 mmol, 1.5 eq) at 0° C. and the reaction mixture was stirred at 0° C. for 1 h. H₂O (20 mL) was added and the mixture was extracted with EA (20 mL×3). The combined organic phase was washed with brine, dried over anhydrous Na₂SO₄ and concentrated under vacuum. The crude residue was purified by combine flash (ME/DCM=0˜10%) to give 1-bromo-6,7,8,9-tetrahydro-5H-benzo[7]annulen-5-amine (Int-50-2) (660 mg, yield 61%). MS(ESI): m/z 224.2 (M-NH2).

### Step 4: Synthesis of tert-butyl 4-(2-((1-bromo-6,7,8,9-tetrahydro-5H-benzo[7]annulen-5-yl)amino)acetamido)-2-((1-((2-(trimethylsilyl)ethoxy)methyl)-1H-pyrrolo[2,3-b]pyridin-5-yl)oxy)benzoate (Int-50-3)

To a solution of 1-bromo-6,7,8,9-tetrahydro-5H-benzo[7]annulen-5-amine (Int-50-2) (800 mg, 1.50 mmol, 1.0 eq), tert-butyl 4-(2-chloroacetamido)-2-((1-((2-(trimethylsilyl)ethoxy) methyl)-1H-pyrrolo[2,3-b]pyridin-5-yl)oxy)benzoate (361 mg, 1.50 mmol, 1.0 eq), NaI (676 mg, 4.50 mmol, 3.0 eq), K₂CO₃ (623 mg, 4.50 mmol, 3.0 eq) in acetonitrile (30 mL) was heated at 90° C. for 5 h. H₂O (40 mL) was added and the mixture was extracted with EA (20 mL×3). The combined organic phase was washed with brine, dried over anhydrous Na₂SO₄ and concentrated under vacuum. The crude residue was purified by combine flash (EA/PE=0˜40%) to give tert-butyl 4-(2-((1-bromo-6,7,8,9-tetrahydro-5H-benzo[7]annulen-5-yl)amino)acetamido)-2-((1-((2-(trimethylsilyl)ethoxy)methyl)-1H-pyrrolo[2,3-b]pyridin-5-yl)oxy)benzoate (Int-50-3) (1.0 g, yield 90%). MS(ESI): m/z 735.0 (M+H+).

### Step 5: Synthesis of tert-butyl 4-(2-(N-(1-bromo-6,7,8,9-tetrahydro-5H-benzo[7]annulen-5-yl)-2-chloroacetamido)acetamido)-2-((1-((2-(trimethylsilyl)ethoxy)methyl)-1H-pyrrolo[2,3-b]pyridin-5-yl)oxy)benzoate (Int-50-4)

To a solution of tert-butyl 4-(2-((1-bromo-6,7,8,9-tetrahydro-5H-benzo[7]annulen-5-yl)amino)acetamido)-2-((1-((2-(trimethylsilyl)ethoxy)methyl)-1H-pyrrolo[2,3-b]pyridin-5-yl)oxy)benzoate (Int-50-3) (1.0 g, 1.36 mmol, 1.0 eq) in DCM (20 mL) was added TEA (0.94 mL, 6.80 mmol, 5.0 eq) and 2-chloroacetyl chloride (0.32 mL, 4.08 mmol, 3.0 eq). The reaction mixture was stirred at rt for 1 h. The organic phase was washed with saturated NaHCO₃ (15 mL×1), brine, dried over anhydrous Na₂SO₄ and concentrated under vacuum. The crude residue was purified by combine flash (EA/PE=0˜40%) to give tert-butyl 4-(2-(N-(1-bromo-6,7,8,9-tetrahydro-5H-benzo[7]annulen-5-yl)-2-chloroacetamido)acetamido)-2-((1-((2-(trimethylsilyl)ethoxy)methyl)-1H-pyrrolo[2,3-b]pyridin-5-yl)oxy)benzoate (Int-50-4) (970 mg, yield 88%). MS(ESI): m/z 811.0 (M+H+).

### Step 6: Synthesis of tert-butyl 4-(4-(1-bromo-6,7,8,9-tetrahydro-5H-benzo[7]annulen-5-yl)-2,5-dioxopiperazin-1-yl)-2-((1-((2-(trimethylsilyl)ethoxy)methyl)-1H-pyrrolo[2,3-b]pyridin-5-yl)oxy)benzoate (Int-50-5)

To a solution of tert-butyl 4-(2-(N-(1-bromo-6,7,8,9-tetrahydro-5H-benzo[7]annulen-5-yl)-2-chloroacetamido)acetamido)-2-((1-((2-(trimethylsilyl)ethoxy)methyl)-1H-pyrrolo[2,3-b]pyridin-5-yl)oxy)benzoate (Int-50-4) (970 mg, 1.19 mmol, 1.0 eq), NaI (537 mg, 3.58 mmol, 3.0 eq), K₂CO₃ (495 mg, 3.58 mmol, 3.0 eq) in acetonitrile (15 mL) was heated at 90° C. for 5 h. EA (50 mL) was added and the organic phase was washed with H₂O (20 mL×1), brine, dried over anhydrous Na₂SO₄ and concentrated under vacuum. The crude residue was purified by combine flash (EA/PE=0˜40%) to give tert-butyl 4-(4-(1-bromo-6,7,8,9-tetrahydro-5H-benzo[7]annulen-5-yl)-2,5-dioxopiperazin-1-yl)-2-((1-((2-(trimethylsilyl)ethoxy)methyl)-1H-pyrrolo[2,3-b]pyridin-5-yl)oxy)benzoate (Int-50-5) (900 mg, yield 97%). MS(ESI): m/z 775.0 (M+H+).

### Step 7: Synthesis of tert-butyl 4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-9-3)

To a solution of tert-butyl 4-(4-(1-bromo-6,7,8,9-tetrahydro-5H-benzo[7]annulen-5-yl)-2,5-dioxopiperazin-1-yl)-2-((1-((2-(trimethylsilyl)ethoxy)methyl)-1H-pyrrolo[2,3-b]pyridin-5-yl)oxy)benzoate (Int-50-5) (900 mg, 1.16 mmol, 1.0 eq) in THE (12 mL) was added 1M BH₃·THF (12.0 mL, 12.0 mmol, 10.0 eq) and the mixture was stirred at rt for 2 h. MeOH (20 mL) was added slowly and the mixture was stirred at 60° C. overnight. The solvent was removed under vacuum. H₂O (20 mL) was added slowly and the mixture was extracted with EA (30 mL×3). The combined organic phase was wished with brine, dried over anhydrous Na₂SO₄ and concentrated under vacuum. The crude residue was purified by combine flash (EA/PE=0˜40%) to give tert-butyl 4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-9-3) (510 mg, yield 59%). MS(ESI): m/z 747.1 (M+H+).

### Step 8: Synthesis of tert-butyl 4-(4-(1-(prop-1-en-2-yl)-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-50-6)

To a solution of tert-butyl 4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-9-3) (200 mg, 0.27 mmol, 1.0 eq), 4,4,5,5-tetramethyl-2-(prop-1-en-2-yl)-1,3,2-dioxaborolane (135 mg, 0.80 mmol, 3.0 eq), Pd(PPh₂)Cl₂ (10 mg, 0.03 mmol, 0.1 eq), Cs₂CO₃ (174 mg, 0.53 mmol, 2.0 eq) in dioxane (5 mL) and H₂O (0.5 mL) was heated to 80° C. for 6 h. EA (30 mL) was added and the mixture was washed with H₂O (20 mL×1), brine, dried over anhydrous Na₂SO₄ and concentrated under vacuum. The crude residue was purified by combine flash (EA/PE=0˜40%) to give tert-butyl 4-(4-(1-(prop-1-en-2-yl)-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-50-6) (180 mg, yield 94%). MS(ESI): m/z 709.2 (M+H+).

### Step 9: Synthesis of 2-((1H-pyrrolo[2,3-b]pyridin-5-yl)oxy)-4-(4-(1-(prop-1-en-2-yl)-6,7,8,9-tetrahydro-5H-benzo[7]annulen-5-yl)piperazin-1-yl)benzoic acid (Int-50-7)

To a solution of tert-butyl 4-(4-(1-(prop-1-en-2-yl)-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-50-6) (100 mg, 0.14 mmol) in DCM (4 mL) was added TFA (2 mL). The reaction mixture was stirred at RT for 1 h. LC-MS monitored and the starting material was consumed completely. The mixture was concentrated under vacuum. The residue was dissolved in MeOH (4 mL) and then K₂CO₃ aqueous solution was added to adjust pH>8. The mixture was stirred at rt overnight. 1 M HCl aqueous solution was added to adjust pH to 6 and the mixture was extracted with EA (20 mL×3). The combined organic phase was washed with brine, dried over anhydrous Na₂SO₄ and concentrated to give 2-((1H-pyrrolo[2,3-b]pyridin-5-yl)oxy)-4-(4-(1-(prop-1-en-2-yl)-6,7,8,9-tetrahydro-5H-benzo[7]annulen-5-yl)piperazin-1-yl)benzoic acid (Int-50-7) (74 mg, yield 100%). MS(ESI): m/z 523.3 (M+H+).

### Step 10: Synthesis of 2-((1H-pyrrolo[2,3-b]pyridin-5-yl)oxy)-N-((3-nitro-4-(((tetrahydro-2H-pyran-4-yl)methyl)amino)phenyl)sulfonyl)-4-(4-(1-(prop-1-en-2-yl)-6,7,8,9-tetrahydro-5H-benzo[7]annulen-5-yl)piperazin-1-yl)benzamide (Compound 50)

To a solution of 2-((1H-pyrrolo[2,3-b]pyridin-5-yl)oxy)-4-(4-(1-(prop-1-en-2-yl)-6,7,8,9-tetrahydro-5H-benzo[7]annulen-5-yl)piperazin-1-yl)benzoic acid (Int-50-7) (74 mg, 0.14 mmol, 1.0 eq), 3-nitro-4-(((tetrahydro-2H-pyran-4-yl)methyl)amino)benzenesulfonamide (Int-9-7) (44 mg, 0.14 mmol, 1.0 eq) in DMF (2 mL) was added DIPEA (55 mg, 0.42 mmol, 3.0 eq), DMAP (52 mg, 0.42 mmol, 3.0 eq) and EDCI (35 mg, 0.18 mmol, 1.3 eq). The reaction mixture was stirred at RT overnight. EA (20 mL) was added and the mixture was washed with water (10 mL×3), brine, dried over anhydrous Na₂SO₄ and concentrated. The crude residue was purified by Prep-TLC (ME/DCM=1/20) to give 2-((1H-pyrrolo[2,3-b]pyridin-5-yl)oxy)-N-((3-nitro-4-(((tetrahydro-2H-pyran-4-yl)methyl)amino)phenyl)sulfonyl)-4-(4-(1-(prop-1-en-2-yl)-6,7,8,9-tetrahydro-5H-benzo[7]annulen-5-yl)piperazin-1-yl)benzamide (Compound 50) (19.6 mg, yield 17%). MS(ESI): m/z 820.2 (M+H+).

### Example 17: Synthesis of Compound 47

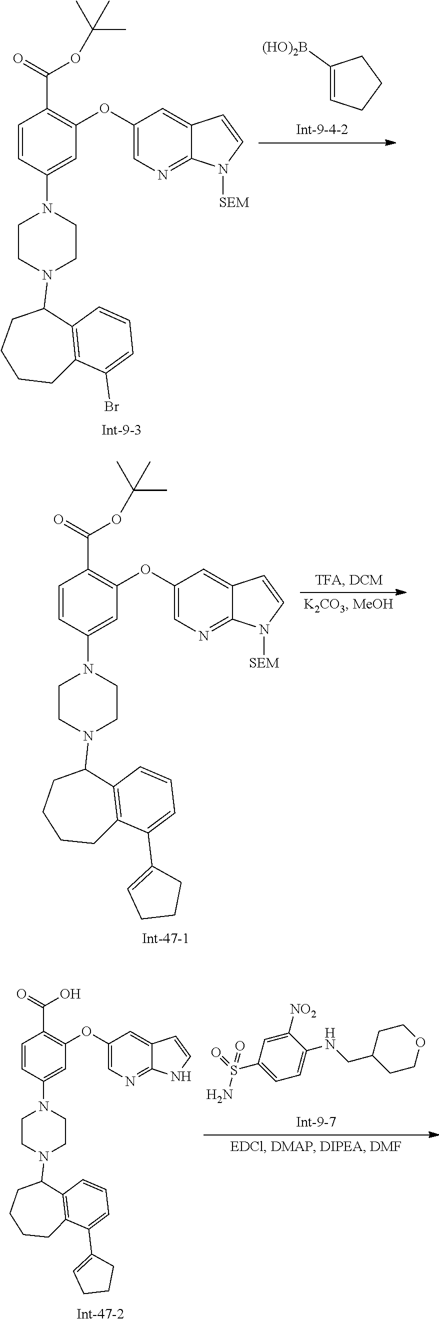

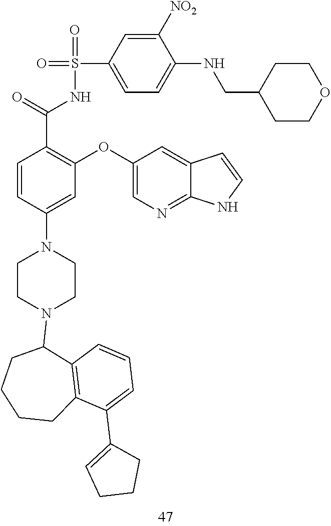 [SMILES: O=C(NS(=O)(=O)c1ccc(NCC2CCOCC2)c([N+](=O)[O-])c1)c1ccc(N2CCN(C3CCCCc4c(C5=CCCC5)cccc43)CC2)cc1Oc1cnc2[nH]ccc2c1]

### Step 1: Synthesis of tert-butyl 4-(4-(1-(cyclopent-1-en-1-yl)-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-47-1)

To a solution of tert-butyl 4-(4-(1-bromo-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-9-3) (200 mg, 0.27 mmol, 1.0 eq), 4,4,5,5-tetramethyl-2-(prop-1-en-2-yl)-1,3,2-dioxaborolane (90 mg, 0.80 mmol, 3.0 eq), Pd(PPh₂)Cl₂ (10 mg, 0.03 mmol, 0.1 eq), Cs₂CO₃ (174 mg, 0.53 mmol, 2.0 eq) in dioxane (5 mL) and H₂O (0.5 mL) was heated to 80° C. for 6 h. EA (30 mL) was added and the mixture was washed with H₂O (20 mL×1), brine, dried over anhydrous Na₂SO₄ and concentrated under vacuum. The crude residue was purified by combine flash (EA/PE=0˜40%) to give tert-butyl 4-(4-(1-(cyclopent-1-en-1-yl)-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-47-1) (150 mg, yield 76%). MS(ESI): m/z 735.2 (M+H+).

### Step 2: Synthesis of 2-((1H-pyrrolo[2,3-b]pyridin-5-yl)oxy)-4-(4-(1-(cyclopent-1-en-1-yl)-6,7,8,9-tetrahydro-5H-benzo[7]annulen-5-yl)piperazin-1-yl)benzoic acid (Int-47-2)

To a solution of tert-butyl 4-(4-(1-(cyclopent-1-en-1-yl)-6,7,8,9-tetrahydro-5H-benzo[7]annulen-5-yl)piperazin-1-yl)-2-((1-((2-(trimethylsilyl)ethoxy)methyl)-1H-pyrrolo[2,3-b]pyridin-5-yl)oxy)benzoate (Int-47-1) (50 mg, 0.068 mmol) in DCM (4 mL) was added TFA (2 mL). The reaction mixture was stirred at rt for 1 h. LC-MS monitored and the starting material was consumed completely. The mixture was concentrated under vacuum. The residue was dissolved in MeOH (4 mL) and then K₂CO₃ aqueous solution was added to adjust pH>8. The mixture was stirred at RT overnight. 1 M HCl aqueous solution was added to adjust pH to 6 and the mixture was extracted with EA (20 mL×3). The combined organic phase was washed with brine, dried over anhydrous Na₂SO₄ and concentrated to give 2-((1H-pyrrolo[2,3-b]pyridin-5-yl)oxy)-4-(4-(1-(cyclopent-1-en-1-yl)-6,7,8,9-tetrahydro-5H-benzo[7]annulen-5-yl)piperazin-1-yl)benzoic acid (Int-47-2) (37 mg, yield 100%). MS(ESI): m/z 549.2 (M+H+).

### Step 3: Synthesis of 2-((1H-pyrrolo[2,3-b]pyridin-5-yl)oxy)-4-(4-(1-(cyclopent-1-en-1-yl)-6,7,8,9-tetrahydro-5H-benzo[7]annulen-5-yl)piperazin-1-yl)-N-((3-nitro-4-(((tetrahydro-2H-pyran-4-yl)methyl)amino)phenyl)sulfonyl)benzamide (Compound 47)

To a solution of 2-((1H-pyrrolo[2,3-b]pyridin-5-yl)oxy)-4-(4-(1-(prop-1-en-2-yl)-6,7,8,9-tetrahydro-5H-benzo[7]annulen-5-yl)piperazin-1-yl)benzoic acid (Int-47-2) (37 mg, 0.067 mmol, 1.0 eq), 3-nitro-4-(((tetrahydro-2H-pyran-4-yl)methyl)amino)benzenesulfonamide (Int-9-7) (21 mg, 0.067 mmol, 1.0 eq) in DMF (2 mL) was added DIPEA (26 mg, 0.20 mmol, 3.0 eq), DMAP (25 mg, 0.20 mmol, 3.0 eq) and EDCI (17 mg, 0.088 mmol, 1.3 eq). The reaction mixture was stirred at RT overnight. EA (20 mL) was added and the mixture was washed with water (10 mL×3), brine, dried over anhydrous Na₂SO₄ and concentrated. The crude residue was purified by Prep-TLC (ME/DCM=1/20) to give 2-((1H-pyrrolo[2,3-b]pyridin-5-yl)oxy)-4-(4-(1-(cyclopent-1-en-1-yl)-6,7,8,9-tetrahydro-5H-benzo[7]annulen-5-yl)piperazin-1-yl)-N-((3-nitro-4-(((tetrahydro-2H-pyran-4-yl)methyl)amino)phenyl)sulfonyl)benzamide (Compound 47) (5 mg, yield 90). MS(ESI): m/z 846.3 (M+H+).

The following compounds were prepared according to the above described methods using different starting materials.

| Compound | | MS(ESI): m/z |
|---|---|---|
| No. | IUPAC Name | (M + H⁺) |
| 49 | 2-((1H-pyrrolo[2,3-b]pyridin-5-yl)oxy)-N-((3- | 857.3 |
| | nitro-4-(((tetrahydro-2H-pyran-4- | |
| | yl)methyl)amino)phenyl)sulfonyl)-4-(4-(1- | |
| | (pyridin-3-yl)-6,7,8,9-tetrahydro-5H- | |
| | benzo[7]annulen-5-yl)piperazin-1-yl)benzamide | |
| 52 | 4-(4-(1-(1H-pyrazol-4-yl)-6,7,8,9-tetrahydro-5H- | 846.3 |
| | benzo[7]annulen-5-yl)piperazin-1-yl)-2-((1H- | |
| | pyrrolo[2,3-b]pyridin-5-yl)oxy)-N-((3-nitro-4- | |
| | (((tetrahydro-2H-pyran-4- | |
| | yl)methyl)amino)phenyl)sulfonyl)benzamide | |
| 54 | 4-(4-(1-(1H-pyrazol-3-yl)-6,7,8,9-tetrahydro-5H- | 846.3 |
| | benzo[7]annulen-5-yl)piperazin-1-yl)-2-((1H- | |
| | pyrrolo[2,3-b]pyridin-5-yl)oxy)-N-((3-nitro-4- | |
| | (((tetrahydro-2H-pyran-4- | |
| | yl)methyl)amino)phenyl)sulfonyl)benzamide | |

### Example 18: Synthesis of Compound 51 & 55

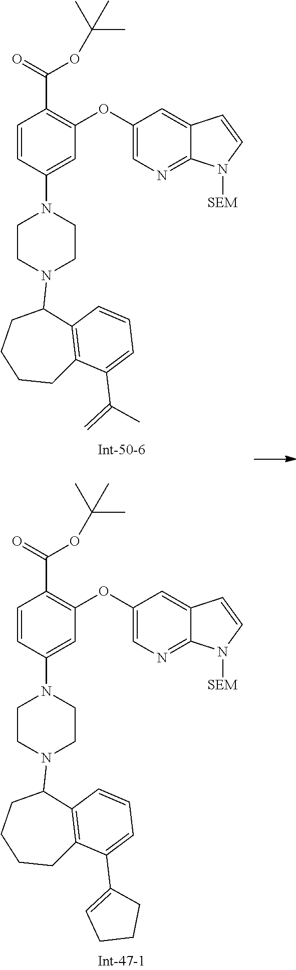 [SMILES: C=C(C)c1cccc2c1CCCCC2N1CCN(c2ccc(C(=O)OC(C)(C)C)c(Oc3cnc4c(ccn4COCC[Si](C)(C)C)c3)c2)CC1 ; CC(C)(C)OC(=O)c1ccc(N2CCN(C3CCCCc4c(C5=CCCC5)cccc43)CC2)cc1Oc1cnc2c(ccn2COCC[Si](C)(C)C)c1]

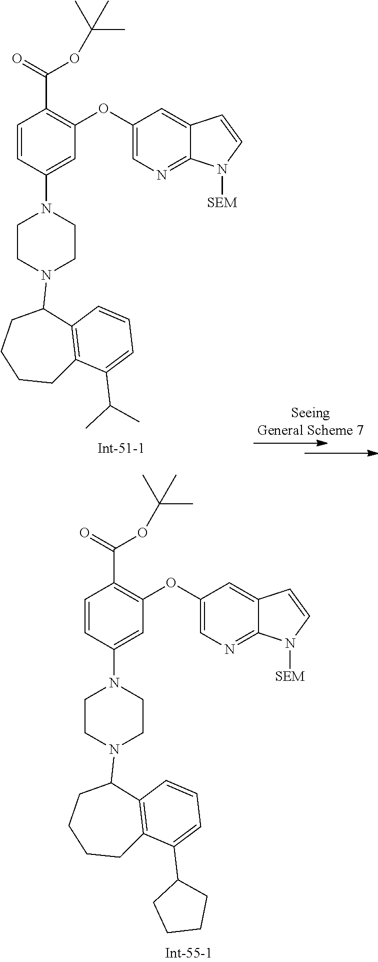 [SMILES: CC(C)c1cccc2c1CCCCC2N1CCN(c2ccc(C(=O)OC(C)(C)C)c(Oc3cnc4c(ccn4COCC[Si](C)(C)C)c3)c2)CC1 ; CC(C)(C)OC(=O)c1ccc(N2CCN(C3CCCCc4c(C5CCCC5)cccc43)CC2)cc1Oc1cnc2c(ccn2COCC[Si](C)(C)C)c1]

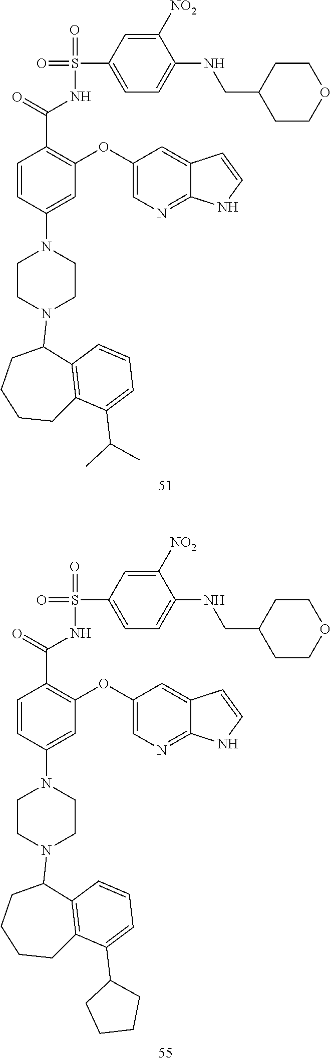 [SMILES: CC(C)c1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1 ; O=C(NS(=O)(=O)c1ccc(NCC2CCOCC2)c([N+](=O)[O-])c1)c1ccc(N2CCN(C3CCCCc4c(C5CCCC5)cccc43)CC2)cc1Oc1cnc2[nH]ccc2c1]

### Step 1: Synthesis of Int-51-1 & Int-55-1

To a solution of Int-50-6 or Int-47-1 (0.14 mmol) in MeOH (20 mL) was added Pd/C (100 mg). The reaction mixture was stirred at rt under H₂ atmosphere for 1 h. LC-MS monitored and the starting material was consumed completely. Filtered and concentrated the solvent to give Int-51-1 or Int-55-1. Int-51-1: MS(ESI): m/z 711.2 (M+H+). Int-55-1: MS(ESI): m/z 737.2 (M+H+).

### Step 2: Synthesis of Compound 51 & 55

Seeing the preparation of Formula 7 in General Scheme 7.

| Compound | | MS(ESI): m/z |
|---|---|---|
| No. | IUPAC Name | (M + H⁺) |
| 51 | 2-((1H-pyrrolo[2,3-b]pyridin-5-yl)oxy)-4-(4-(1- | 822.3 |
| | isopropyl-6,7,8,9-tetrahydro-5H-benzo[7]annulen- | |
| | 5-yl)piperazin-1-yl)-N-((3-nitro-4-(((tetrahydro- | |
| | 2H-pyran-4- | |
| | yl)methyl)amino)phenyl)sulfonyl)benzamide | |
| 55 | 2-((1H-pyrrolo[2,3-b]pyridin-5-yl)oxy)-4-(4-(1- | 848.3 |
| | cyclopentyl-6,7,8,9-tetrahydro-5H- | |
| | benzo[7]annulen-5-yl)piperazin-1-yl)-N-((3-nitro- | |
| | 4-(((tetrahydro-2H-pyran-4- | |
| | yl)methyl)amino)phenyl)sulfonyl)benzamide | |

### Example 19: Synthesis of Compound 61A & 61B

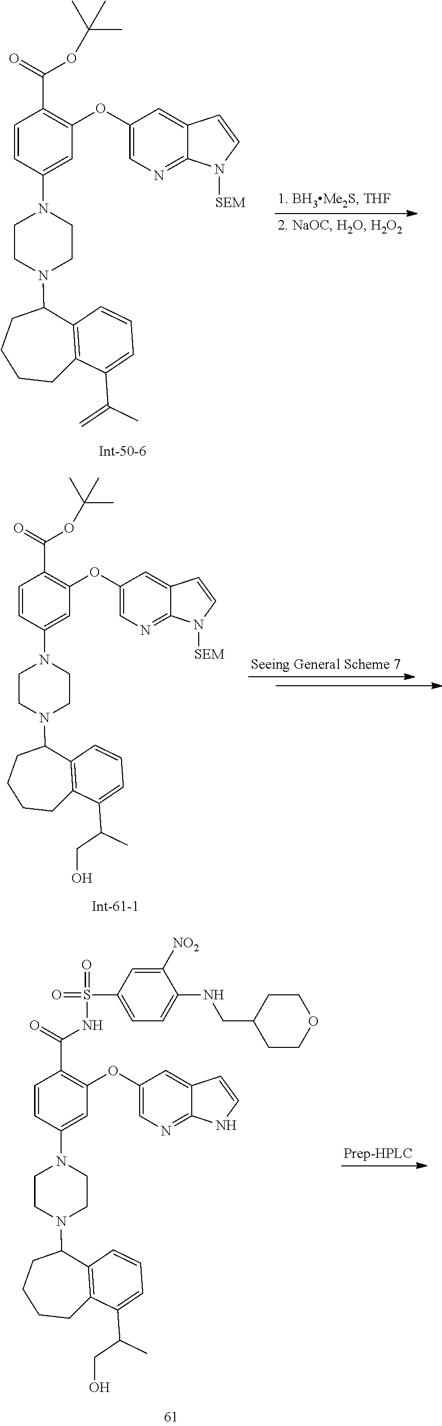 [SMILES: C=C(C)c1cccc2c1CCCCC2N1CCN(c2ccc(C(=O)OC(C)(C)C)c(Oc3cnc4c(ccn4COCC[Si](C)(C)C)c3)c2)CC1 ; CC(CO)c1cccc2c1CCCCC2N1CCN(c2ccc(C(=O)OC(C)(C)C)c(Oc3cnc4c(ccn4COCC[Si](C)(C)C)c3)c2)CC1 ; CC(CO)c1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1]

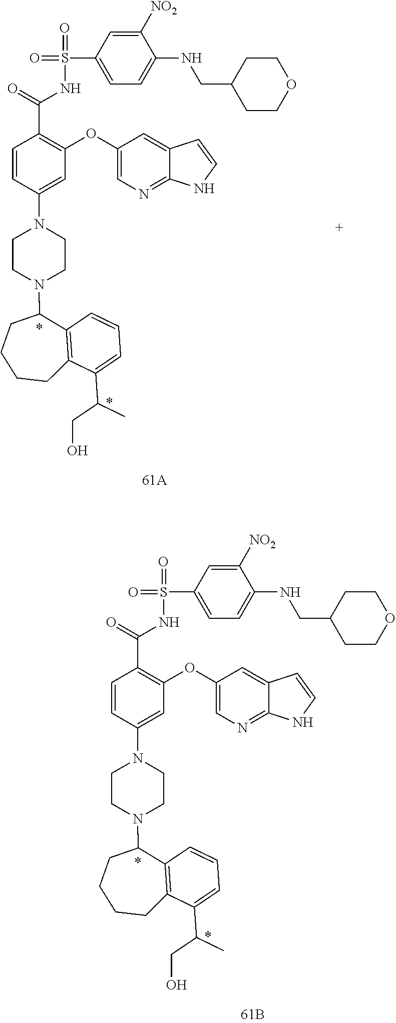 [SMILES: CC(CO)c1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1 ; CC(CO)c1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1]

### Step 1: Synthesis of Int-61-1

To a solution of Int-50-6 (100 mg, 0.14 mmol, 1.0 eq) in THE (5 mL) was added 2 M BH₃Me₂S (0.14 mL, 0.28 mmol, 2.0 eq) at 0° C. and the reaction mixture was stirred at rt for 7 h. LC-MS monitored and the starting material was consumed completely. A mixture of saturated NaOAc aqueous solution (7 mL) and 30% H₂O 2 (3 mL) was added slowly. The mixture was stirred at rt overnight. H₂O (30 mL) was added and the mixture was extracted with EA (20 mL×3). The combined organic phase was washed with brine, dried over anhydrous Na₂SO₄ and concentrated under vacuum. The residue was purified by combine flash (EA/PE=0˜60%) to give Int-61-1 (70 mg, yield 68%). MS(ESI): m/z 727.3 (M+H+).

### Step 2: Synthesis of Compound 61

Seeing the preparation of Formula 7 in General Scheme 7 to give Compound 61. Compound 61 was purified by Prep-HPLC to give the Compound 61A and 61B. MS(ESI): m/z 838.3 (M+H+).

| Compound | | MS(ESI): m/z |
|---|---|---|
| No. | IUPAC Name | (M + H⁺) |
| 61A | (R)-2-((1H-pyrrolo[2,3-b]pyridin-5-yl)oxy)-4- | 838.3 |
| | (4-(1-(1-hydroxypropan-2-yl)-6,7,8,9- | |
| | tetrahydro-5H-benzo[7]annulen-5-yl)piperazin- | |
| | 1-yl)-N-((3-nitro-4-(((tetrahydro-2H-pyran-4- | |
| | yl)methyl)amino)phenyl)sulfonyl)benzamide | |
| 61B | (S)-2-((1H-pyrrolo[2,3-b]pyridin-5-yl)oxy)-4-(4- | 838.3 |
| | (1-(1-hydroxypropan-2-yl)-6,7,8,9-tetrahydro- | |
| | 5H-benzo[7]annulen-5-yl)piperazin-1-yl)-N-((3- | |
| | nitro-4-(((tetrahydro-2H-pyran-4- | |
| | yl)methyl)amino)phenyl)sulfonyl)benzamide | |

### Example 20: Synthesis of Compound 62 & 63

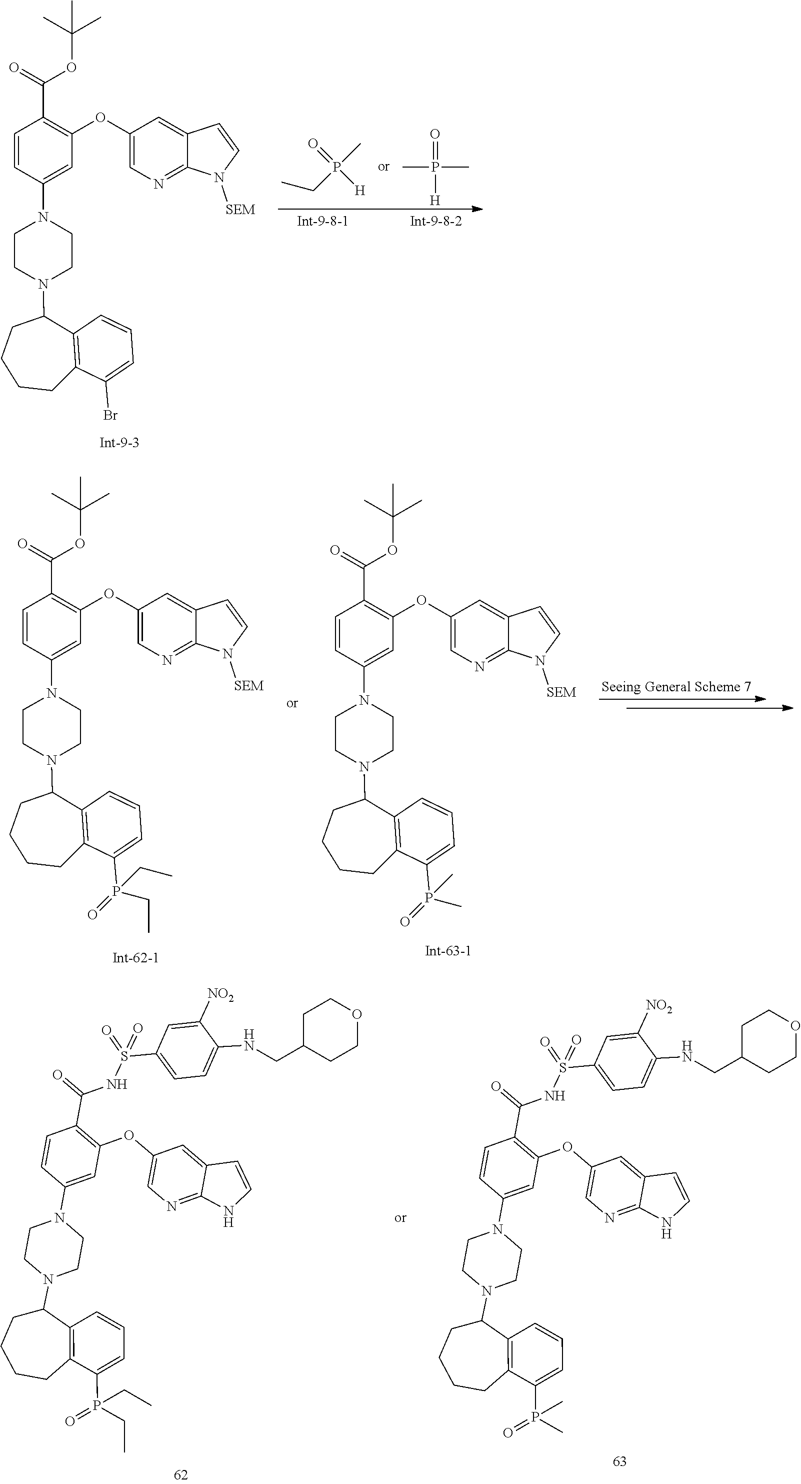

### Step 1: Synthesis of Compound 62-1 & 63-1

To a stirred solution of Int-9-3 (400 mg, 0.53 mmol, 1.0 eq), Xantphos (61 mg, 0.11 mmol, 0.2 eq) and Pd(OAc)₂ (12 mg, 0.05 mmol, 0.1 eq), K₃PO₄ (170 mg, 0.80 mmol, 1.5 eq) in DMF (5 mL) was added diethylphosphine oxide (114 mg, 1.07 mmol, 2.0 eq) or dimethylphosphine oxide (83 mg, 1.07 mmol, 2.0 eq). The resulting mixture was stirred at 130° C. for 4 h under microwave. The reaction mixture was filtered off. The filtrate was diluted with water (200 mL) and was extracted with EtOAc (100 mL×3). The combined organic extracts were concentrated. The residue was purified by flash chromatography (0 to 20% MeOH in DCM) to give Int-62-1 (390 mg, 94%) or Int-63-1 (320 mg, 80%). Int-62-1: MS(ESI): m/z 773.3 (M+H⁺), Int-63-1: MS(ESI): m/z 745.3 (M+H⁺).

### Step 2: Synthesis of Compound 62 & 63

Seeing the preparation of Formula 7 in General Scheme 7.

| Compound | | MS(ESI): m/z |
|---|---|---|
| No. | IUPAC Name | (M + H⁺) |
| 62 | 2-((1H-pyrrolo[2,3-b]pyridin-5-yl)oxy)-4-(4-(1- | 884.2 |
| | (diethylphosphoryl)-6,7,8,9-tetrahydro-5H- | |
| | benzo[7]annulen-5-yl)piperazin-1-yl)-N-((3-nitro-4- | |
| | (((tetrahydro-2H-pyran-4- | |
| | yl)methyl)amino)phenyl)sulfonyl)benzamide | |
| 63 | 2-((1H-pyrrolo[2,3-b]pyridin-5-yl)oxy)-4-(4-(1- | 856.2 |
| | (dimethylphosphoryl)-6,7,8,9-tetrahydro-5H- | |
| | benzo[7]annulen-5-yl)piperazin-1-yl)-N-((3-nitro-4- | |
| | (((tetrahydro-2H-pyran-4- | |
| | yl)methyl)amino)phenyl)sulfonyl)benzamide | |

### Example 21: Synthesis of Compound 58

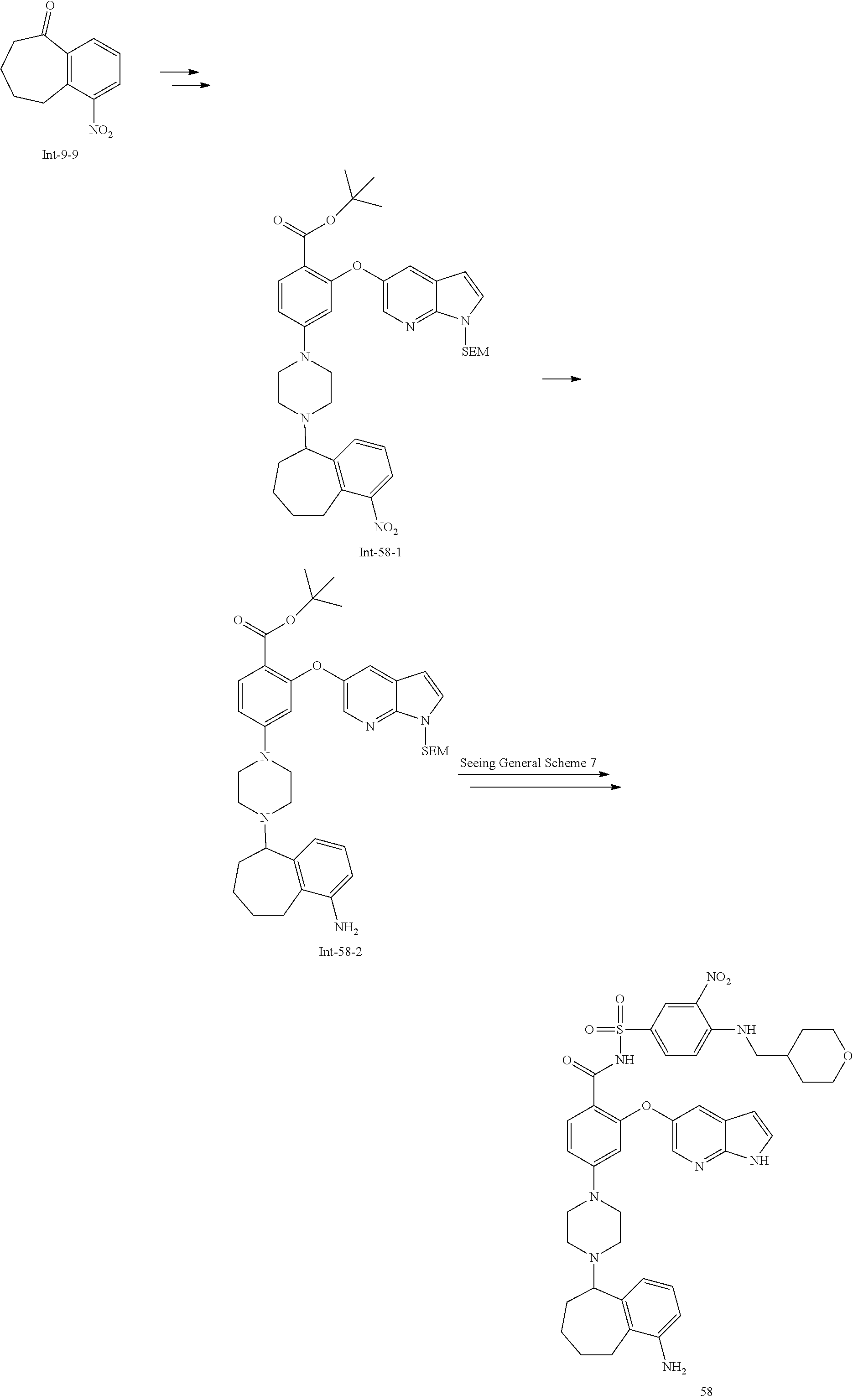 [SMILES: O=C1CCCCc2c1cccc2[N+](=O)[O-] ; CC(C)(C)OC(=O)c1ccc(N2CCN(C3CCCCc4c3cccc4[N+](=O)[O-])CC2)cc1Oc1cnc2c(ccn2COCC[Si](C)(C)C)c1 ; CC(C)(C)OC(=O)c1ccc(N2CCN(C3CCCCc4c(N)cccc43)CC2)cc1Oc1cnc2c(ccn2COCC[Si](C)(C)C)c1 ; Nc1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1]

### Step 1: Synthesis of Compound Int-58-1

Compound Int-58-1 was synthesized as shown in the synthesis of Int-9-3. MS(ESI): m/z 714.2 (M+H⁺).

### Step 2: Synthesis of Compound Int-58-2

To a solution of Compound Int-58-1 (710 mg, 0.99 mmol, 1.0 eq) in EtOH (16 mL) was added saturated NH₄Cl solution (4 mL). Fe (278 mg, 4.97 mmol, 5.0 eq) was added and the mixture was stirred at 90° C. for 1 h. Filter and concentrated the solvent under vacuum. H₂O (40 mL) was added and the mixture was extracted with EA (30 mL×3). The combined organic phase was washed with brine, dried over anhydrous Na₂SO₄ and concentrated to give Int-58-2 (610 mg, yield 90%). MS(ESI): m/z 684.2 (M+H+).

### Step 3: Synthesis of Compound 58

Seeing the preparation of Formula 7 in General Scheme 7. MS(ESI): m/z 795.3 (M+H+).

### Example 22: Synthesis of Compound 56

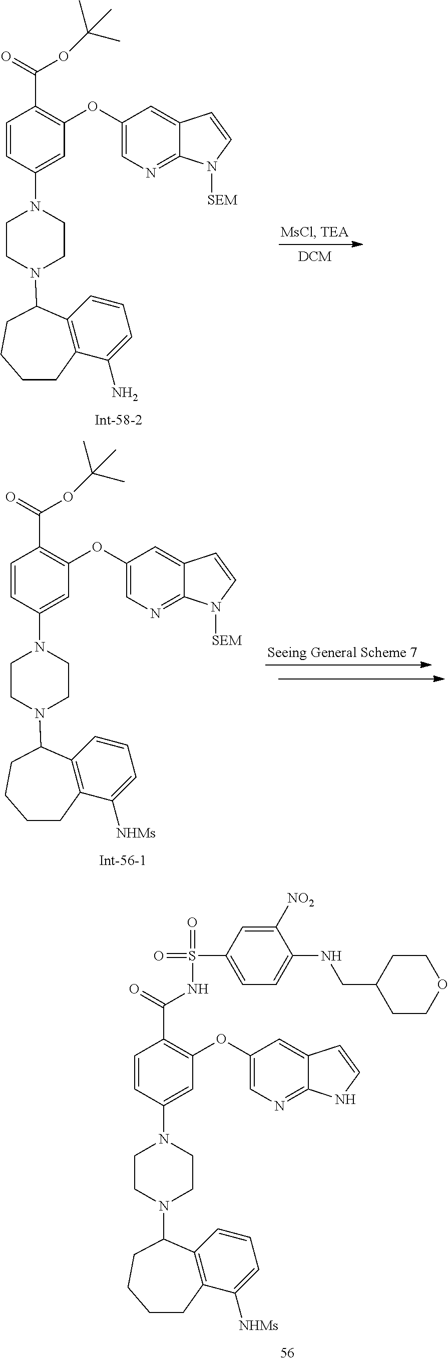

### Step 1: Synthesis of Compound Int-56-1

To a solution of Compound Int-58-2 (100 mg, 0.15 mmol, 1.0 eq) in DCM (5 mL) was added TEA (30 mg, 0.29 mmol, 2.0 eq) and MsCl (17 mg, 0.15 mmol, 1.0 eq). The reaction mixture was stirred at RT for 1 h. H₂O (20 mL) was added and the mixture was extracted with DCM (20 mL×3). The combined organic phase was washed with brine, dried over anhydrous Na₂SO₄ and concentrated under vacuum. The residue was purified by combine flash (EA/PE=0˜70%) to give Int-56-1 (70 mg, yield 63%). MS(ESI): m/z 762.2 (M+H⁺).

### Step 2: Synthesis of Compound 56

Seeing the preparation of Formula 7 in General Scheme 7. MS(ESI): m/z 873.2 (M+H⁺).

### Example 23: Synthesis of Compound 59

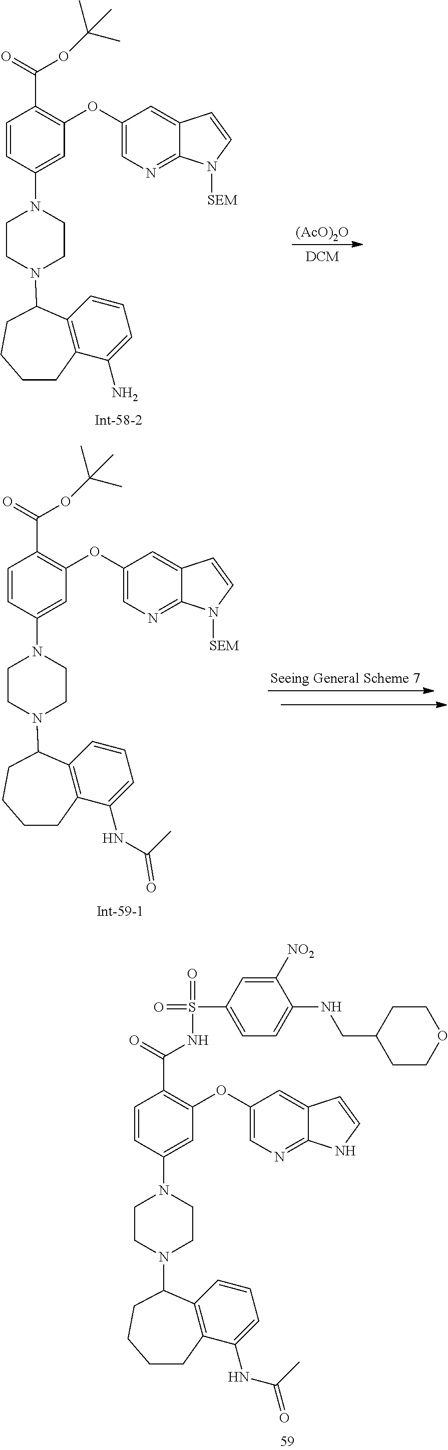 [SMILES: "(AcO)2O" ; CC(C)(C)OC(=O)c1ccc(N2CCN(C3CCCCc4c(N)cccc43)CC2)cc1Oc1cnc2c(ccn2COCC[Si](C)(C)C)c1 ; CC(=O)Nc1cccc2c1CCCCC2N1CCN(c2ccc(C(=O)OC(C)(C)C)c(Oc3cnc4c(ccn4COCC[Si](C)(C)C)c3)c2)CC1 ; CC(=O)Nc1cccc2c1CCCCC2N1CCN(c2ccc(C(=O)NS(=O)(=O)c3ccc(NCC4CCOCC4)c([N+](=O)[O-])c3)c(Oc3cnc4[nH]ccc4c3)c2)CC1]

### Step 1: Synthesis of Int-59-1

To a solution of Int-58-2 (100 mg, 0.15 mmol, 1.0 eq) in DCM (5 mL) was added acetic anhydride (30 mg, 0.29 mmol, 2.0 eq). The reaction mixture was stirred at rt for 1 h. H₂O (20 mL) was added and the mixture was extracted with DCM (20 mL×3). The combined organic phase was washed with brine, dried over anhydrous Na₂SO₄ and concentrated under vacuum. The residue was purified by combine flash (EA/PE=0˜70%) to give Int-59-1 (100 mg, yield 94%). MS(ESI): m/z 726.3 (M+H+).

### Step 2: Synthesis of Compound 59

Seeing the preparation of Formula 7 in General Scheme 7. MS(ESI): m/z 837.3 (M+H+).

The names and MS (ESI) data of Compound 56, 58, and 59 are as follows.

| Compound | | MS(ESI): m/z |
|---|---|---|
| No. | IUPAC Name | (M + H⁺) |
| 56 | 2-((1H-pyrrolo[2,3-b]pyridin-5-yl)oxy)-4-(4-(1- | 873.2 |
| | (methylsulfonamido)-6,7,8,9-tetrahydro-5H- | |
| | benzo[7]annulen-5-yl)piperazin-1-yl)-N-((3- | |
| | nitro-4-(((tetrahydro-2H-pyran-4- | |
| | yl)methyl)amino)phenyl)sulfonyl)benzamide | |
| 58 | 2-((1H-pyrrolo[2,3-b]pyridin-5-yl)oxy)-4-(4-(1- | 795.3 |
| | amino-6,7,8,9-tetrahydro-5H-benzo[7]annulen- | |
| | 5-yl)piperazin-1-yl)-N-((3-nitro-4-(((tetrahydro- | |
| | 2H-pyran-4- | |
| | yl)methyl)amino)phenyl)sulfonyl)benzamide | |
| 59 | 2-((1H-pyrrolo[2,3-b]pyridin-5-yl)oxy)-4-(4-(1- | 837.3 |
| | acetamido-6,7,8,9-tetrahydro-5H- | |
| | benzo[7]annulen-5-yl)piperazin-1-yl)-N-((3- | |
| | nitro-4-(((tetrahydro-2H-pyran-4- | |
| | yl)methyl)amino)phenyl)sulfonyl)benzamide | |

### Example 24: Synthesis of Compound 36

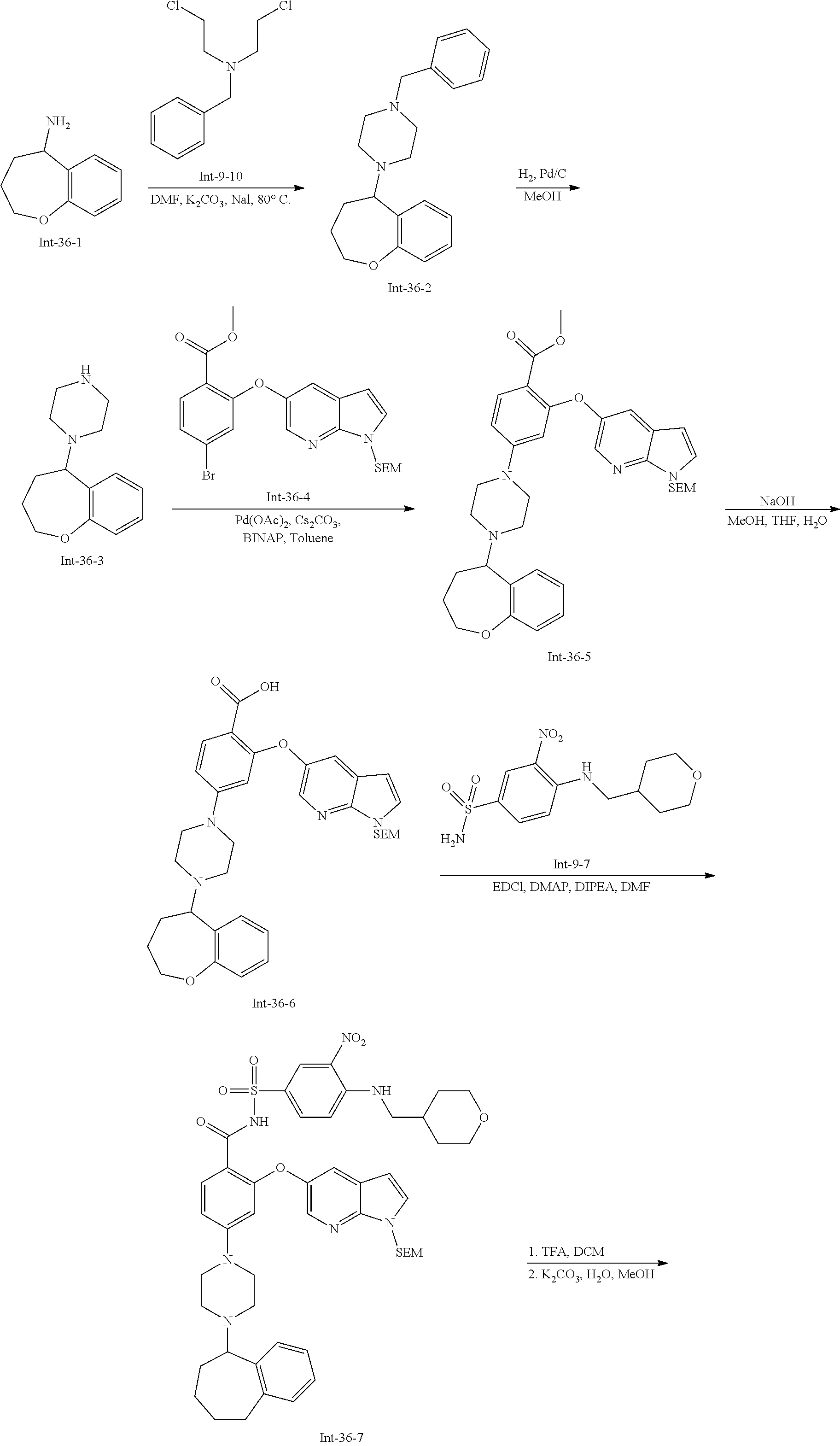

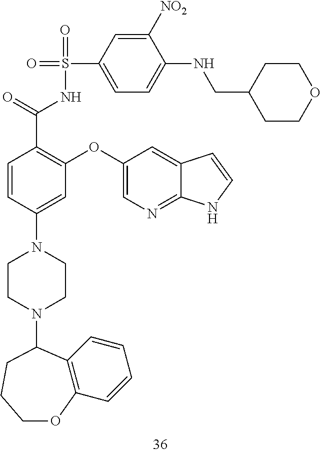 [SMILES: O=C(NS(=O)(=O)c1ccc(NCC2CCOCC2)c([N+](=O)[O-])c1)c1ccc(N2CCN(C3CCCOc4ccccc43)CC2)cc1Oc1cnc2[nH]ccc2c1]

### Step 1: Synthesis of Int-36-2

To a solution of Int-36-1 (320 mg, 1.96 mmol, 1.0 eq), N-benzyl-2-chloro-N-(2-chloroethyl)ethan-1-amine (632 mg, 2.35 mmol, 1.2 eq), K₂CO₃ (1.35 g, 9.80 mmol, 5.0 eq) and NaI (882 mg, 5.88 mmol, 3.0 eq) in DMF (10 mL) was heated at 90° C. overnight. EA (80 mL) was added and the mixture was washed with H₂O (40 mL×3), brine, dried over anhydrous Na₂SO₄ and concentrated under vacuum. The crude residue was purified by combine flash (EA/PE=0˜40%) to give Int-36-2 (377 mg, yield 60%). MS(ESI): m/z 323.3 (M+H+).

### Step 2: Synthesis of Int-36-3

To a solution of Int-36-2 (377 mg, 1.17 mmol) in methanol (30 mL) was added Pd/C (370 mg) and the mixture was stirred at rt under H₂ atmosphere for 7 h. Filtered and concentrated to get the crude product. The crude residue was purified by combine flash (DCM/ME=0˜20%) to give Int-36-3 (200 mg, yield 74%). MS(ESI): m/z 233.3 (M+H+).

### Step 3: Synthesis of Int-36-5

To a solution of Int-36-3 (160 mg, 0.69 mmol, 1.0 eq), Int-36-4 (395 mg, 0.82 mmol, 1.2 eq), Pd(OAc)₂ (154 mg, 0.069 mmol, 0.1 eq), BINAP (43 mg, 0.069 mmol, 0.1 eq) and Cs₂CO₃ (449 mg, 1.38 mmol, 2.0 eq) in toluene (10 mL) was heated to 110° C. for 4 h. H₂O (40 mL) was added and the mixture was extracted with EA (30 mL×3). The combined organic phase was washed with brine, dried over anhydrous Na₂SO₄ and concentrated. The crude residue was purified by combine flash (EA/PE=0˜60%) to give Int-36-5 (400 mg, yield 74%). MS(ESI): m/z 629.3 (M+H+).

### Step 4: Synthesis of Int-36-6

To a solution of Int-36-5 (400 mg, 0.64 mmol, 1.0 eq) in EtOH (20 mL) was added 2M NaOH aqueous solution (3.2 mL, 6.4 mmol, 10 eq). The reaction mixture was stirred at 80° C. overnight. Ethanol was removed under vacuum. HCl aqueous solution was added to adjust pH to 6 and it was extracted with DCM (3×30 mL). The combined organic phase was washed with brine (1×50 mL), dried over sodium sulfate and concentrated under reduced pressure. The residue was purified by flash chromatography (ME/DCM=0˜10%) to give Int-36-6. MS(ESI): m/z 615.2 (M+H+).

### Step 5: Synthesis of Int-36-7

To a solution of Int-36-6 (100 mg, 0.16 mmol, 1.0 eq), Int-9-7 (51 mg, 0.16 mmol, 1.0 eq) in DMF (5 mL) was added DMAP (60 mg, 0.48 mmol, 3.0 eq), DIPEA (62 mg, 0.48 mmol, 3.0 eq) and EDCI (41 mg, 0.21 mmol, 1.3 eq). The reaction mixture was stirred at rt overnight. EA (50 mL) was added and the mixture was washed with water (20 mL×3), brine, dried over anhydrous Na₂SO₄ and concentrated. The crude residue was purified by combine flash (ME/DCM=0˜10%) to give Int-36-7 (100 mg, yield 67%). MS(ESI): m/z 913.2 (M+H+).

### Step 6: Synthesis of Compound 36

To a solution of Int-36-7 (100 mg, 0.11 mmol) in DCM (6 mL) was added TFA (2 mL). The reaction mixture was stirred at rt for 2 h. The mixture was concentrated under vacuum. The residue was dissolved in methanol (10 mL) and K₂CO₃ aqueous solution was added to adjust pH>8. The mixture was stirred at RT overnight. Water (30 mL) was added and the mixture was extracted with EA (20 mL×3). The combined organic phase was washed with brine, dried over anhydrous Na₂SO₄ and concentrated. The crude residue was purified by Prep-TLC (DCM/ME=10/1) to give Compound 36 (19.7 mg, yield 23%). MS(ESI): m/z 782.3 (M+H+).

### Example 25: Synthesis of Compound 45

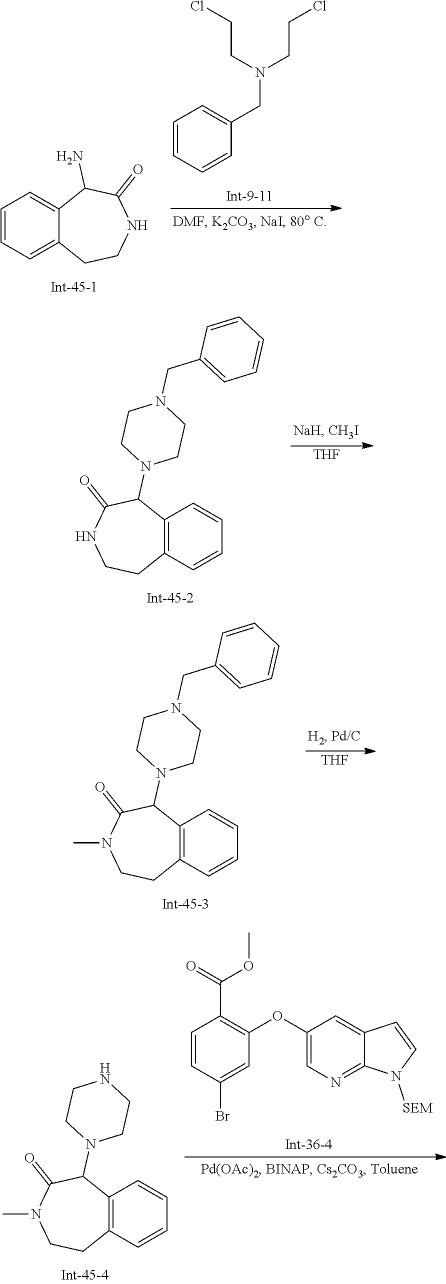

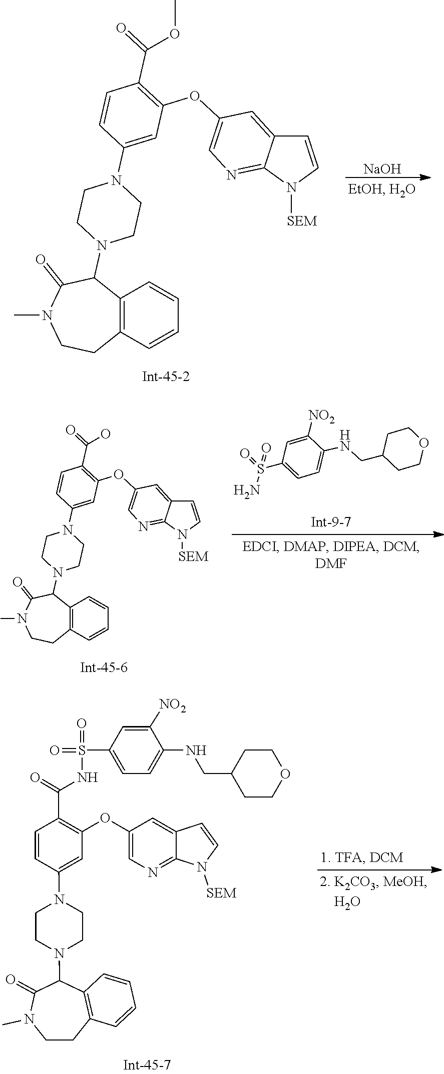

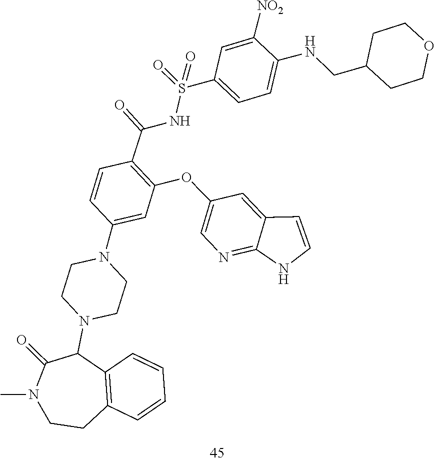 [SMILES: CN1CCc2ccccc2C(N2CCN(c3ccc(C(=O)NS(=O)(=O)c4ccc(NCC5CCOCC5)c([N+](=O)[O-])c4)c(Oc4cnc5[nH]ccc5c4)c3)CC2)C1=O]

### Step 1: Synthesis of Int-45-2

To a solution of Int-45-1 (446 mg, 2.53 mmol, 1.0 eq), N-benzyl-2-chloro-N-(2-chloroethyl)ethan-1-amine (1.02 g, 3.80 mmol, 1.5 eq), K₂CO₃ (1.05 g, 7.59 mmol, 3.3 eq) and NaI (948 mg, 6.33 mmol, 2.5 eq) in DMF (5 mL) was heated at 90° C. for 5 h. EA (30 mL) was added and the mixture was washed with H₂O (20 mL×3), brine, dried over anhydrous Na₂SO₄ and concentrated under vacuum. The crude residue was purified by combine flash (EA/PE=0˜40%) to give Int-45-2 (170 mg, yield 20%). MS(ESI): m/z 336.3 (M+H+).

### Step 2: Synthesis of Int-45-3

To a solution of Int-45-2 (130 mg, 0.39 mmol, 1.0 eq) in THE (4 mL) was added NaH (20 mg, 0.50 mmol, 1.3 eq) at 0° C. The reaction mixture was stirred at 0° C. for 1 h. MeI (83 mg, 0.58 mmol, 1.5 eq) was added and the mixture was stirred at RT overnight. H₂O (20 mL) was added and the mixture was extracted with EA (10 mL×3). The combined organic phase was washed with brine, dried over anhydrous Na₂SO₄ and concentrated under vacuum. The crude residue was purified by combine flash (EA/PE=0˜100%) to give Int-45-3 (90 mg, yield 51%). MS(ESI): m/z 350.1 (M+H+).

### Step 3: Synthesis of Int-45-4

To a solution of Int-45-3 (90 mg, 0.26 mmol) in methanol (10 mL) was added Pd/C (70 mg) and the mixture was stirred at 45° C. under H2 atmosphere for 2 h. Filtered and concentrated to give Int-45-4 (67 mg, yield 100%). MS(ESI): m/z 260.1 (M+H+).

### Step 4: Synthesis of Int-45-5

To a solution of Int-45-4 (70 mg, 0.27 mmol, 1.0 eq), Int-36-4 (155 mg, 0.32 mmol, 1.2 eq), Pd(OAc)₂ (6 mg, 0.027 mmol, 0.1 eq), BINAP (17 mg, 0.027 mmol, 0.1 eq) and Cs₂CO₃ (176 mg, 0.54 mmol, 2.0 eq) in toluene (8 mL) was heated to 110° C. for 6 h. LC-MS monitored and desired product produced. H₂O (20 mL) was added and the mixture was extracted with EA (20 mL×3). The combined organic phase was washed with brine, dried over anhydrous Na₂SO₄ and concentrated. The crude residue was purified by combine flash (EA/PE=0˜60%) to give Int-45-5 (120 mg, yield 68%). MS(ESI): m/z 656.2 (M+H+).

### Step 5: Synthesis of Int-45-6

To a solution of Int-45-5 (120 mg, 0.18 mmol, 1.0 eq) in EtOH (10 mL) was added 3 M NaOH aqueous solution (4 mL). The reaction mixture was stirred at 50° C. for 2 h. LC-MS monitored and the desired product produced. 1 M HCl was added to adjust the pH to 6. The mixture was extracted with EA (30 mL×3). The combined organic phase was washed with brine, dried over anhydrous Na₂SO₄ and concentrated to give Int-45-6 (117 mg, yield 100%). MS(ESI): m/z 642.3 (M+H+).

### Step 6: Synthesis of Int-45-7

To a solution of Int-45-6 (130 mg, 0.20 mmol, 1.0 eq), Int-9-7 (64 mg, 0.20 mmol, 1.0 eq) in DMF (2 mL) was added DIPEA (0.1 mL, 0.61 mmol, 3.0 eq), DMAP (74 mg, 0.61 mmol) and EDCI (51 mg, 0.26 mmol, 1.3 eq). The reaction mixture was stirred at RT for 24 h. EA (30 mL) was added and the mixture was washed with water (20 mL×3), brine, dried over anhydrous Na₂SO₄ and concentrated. The crude residue was purified by combine flash (ME/DCM=0˜10%) to give Int-45-7 (110 mg, yield 58%). MS(ESI): m/z 939.3 (M+H+).

### Step 7: Synthesis of Compound 45

To a solution of Int-45-7 (110 mg, 0.12 mmol) in DCM (6 mL) was added TFA (2 mL). The reaction mixture was stirred at rt for 2 h. LC-MS monitored and the starting material was consumed completely. The mixture was concentrated under vacuum. The residue was dissolved in MeOH (8 mL) and then K₂CO₃ aqueous solution was added to adjust pH>8. The mixture was stirred at rt overnight. H₂O (20 mL) and the mixture was extracted with EA (20 mL×3). The combined organic phase was washed with brine, dried over anhydrous Na₂SO₄ and concentrated. The residue was purified by combine flash (ME/DCM=0˜10%) to give Compound 45 (37.9 mg, yield 40%). MS(ESI): m/z 809.3 (M+H+).

### Example 26: Synthesis of Compound 40

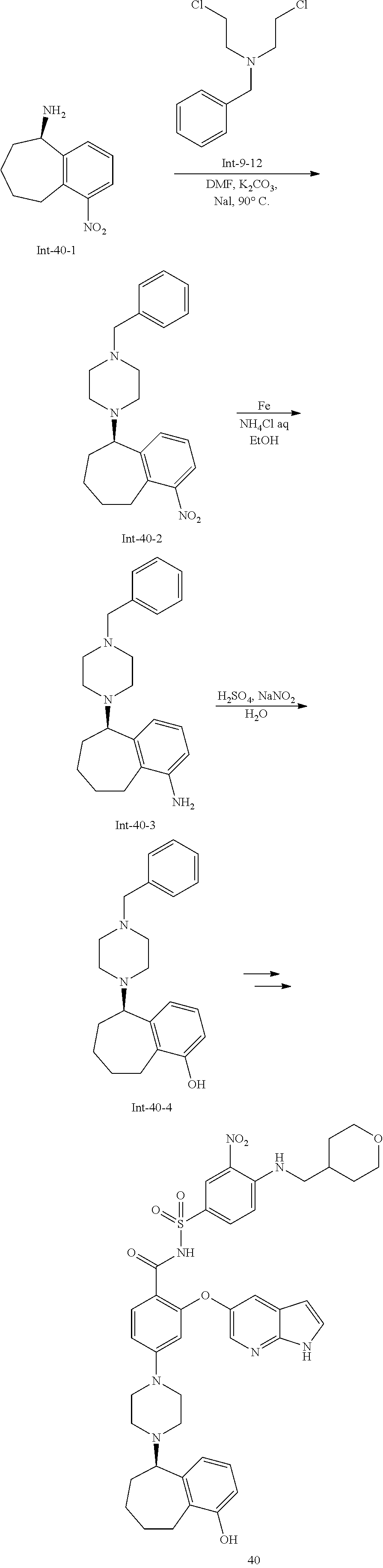

### Step 1: Synthesis of Int-40-2

Using Int-40-1 as starting material, Int-40-2 is synthesize as shown as Int-36-2. Yield: 38%. MS(ESI): m/z 366.1 (M+H+).

### Step 2: Synthesis of Int-40-3

To a solution of Int-40-2 (87 mg, 0.24 mmol, 1.0 eq) in EtOH (10 mL) was added saturated NH₄Cl solution (4 mL). Fe (76 mg, 1.19 mmol, 5.0 eq) was added and the mixture was stirred at 90° C. for 1 h. H₂O (40 mL) was added and the mixture was extracted with EA (30 mL×3). The combined organic phase was washed with brine, dried over anhydrous Na₂SO₄ and concentrated to give Int-40-3 (80 mg, yield 100%). MS(ESI): m/z 336.1 (M+H+).

### Step 3: Synthesis of Int-40-4

To a solution of Int-40-3 (80 mg, 0.24 mmol, 1.0 eq) in H2SO4 aqueous solution (6%, 4 mL) was added NaNO2 (25 mg, 0.36 mmol, 1.5 eq) in H₂O (1 mL) in 0° C. The mixture was stirred at 45° C. for 1 h. H₂O (20 mL) was added and the mixture was extracted with EA (20 mL×3). The combined organic phase was washed with brine, dried over anhydrous Na₂SO₄ and concentrated. The crude residue was purified by Prep-TLC (EA/PE=1/2) to give Int-40-4 (40 mg, yield 50%). MS(ESI): m/z 337.2 (M+H+).

### Step 4: Synthesis of Compound 40

Seeing preparation of Compound 36. MS(ESI): m/z 796.2 (M+H+).

The names and MS (ESI) data of Compound 36, 45 and 40 are shown as follows.

| Compound | | MS(ESI): m/z |
|---|---|---|
| No. | IUPAC Name | (M + H⁺) |
| 36 | 2-((1H-pyrrolo[2,3-b]pyridin-5-yl)oxy)-N-((3-nitro-4- | 782.3 |
| | (((tetrahydro-2H-pyran-4- | |
| | yl)methyl)amino)phenyl)sulfonyl)-4-(4-(2,3,4,5- | |
| | tetrahydrobenzo[b]oxepin-5-yl)piperazin-1- | |
| | yl)benzamide | |
| 45 | 2-((1H-pyrrolo[2,3-b]pyridin-5-yl)oxy)-4-(4-(3- | 809.3 |
| | methyl-2-oxo-2,3,4,5-tetrahydro-1H-benzo[d]azepin-1- | |
| | yl)piperazin-1-yl)-N-((3-nitro-4-(((tetrahydro-2H- | |
| | pyran-4-yl)methyl)amino)phenyl)sulfonyl)benzamide | |
| 40 | (R)-2-((1H-pyrrolo[2,3-b]pyridin-5-yl)oxy)-4-(4-(1- | 796.2 |
| | hydroxy-6,7,8,9-tetrahydro-5H-benzo[7]annulen-5- | |
| | yl)piperazin-1-yl)-N-((3-nitro-4-(((tetrahydro-2H- | |
| | pyran-4-yl)methyl)amino)phenyl)sulfonyl)benzamide | |

### Example 27: Synthesis of Compound 60

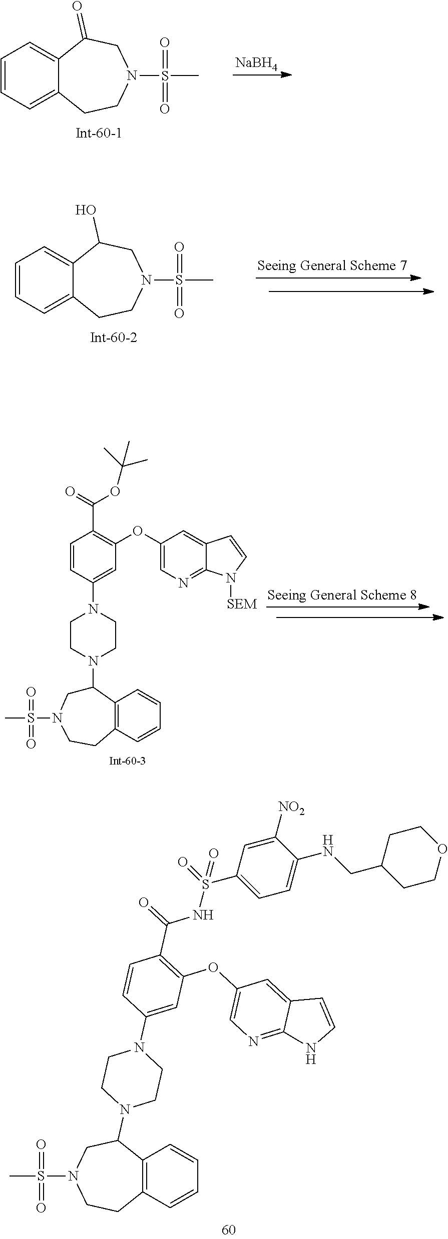

### Step 1: Synthesis of Int-60-2

To a solution of Int-60-1 (1.0 g, 4.18 mmol, 1.0 eq) in methanol (20 mL) was added NaBH₄ (237 mg, 6.27 mmol, 1.5 eq) at 0° C. and the reaction mixture was stirred at 0° C. for 2 h. H₂O (10 mL) was added to quench the reaction. The solvent was removed under vacuum. H₂O (50 mL) was added and the mixture was extracted with EA (30 mL×3). The combined organic phase was washed with brine, dried over anhydrous Na₂SO₄ and concentrated under vacuum. The crude residue was purified by combine flash (EA/PE=0˜30%) to give Int-60-2 (800 mg, yield 79%). MS(ESI): m/z 224.2 (M-OH).

### Step 2: Synthesis of Int-60-3

Seeing the preparation of Int-7-15 in General Scheme 7. MS(ESI): m/z 748.2 (M+H+).

### Step 3: Synthesis of Compound 60

Seeing the preparation of Formula 8 in General Scheme 8. MS(ESI): m/z 858.0 (M+H+).

### Example 28: Synthesis of Compound 76

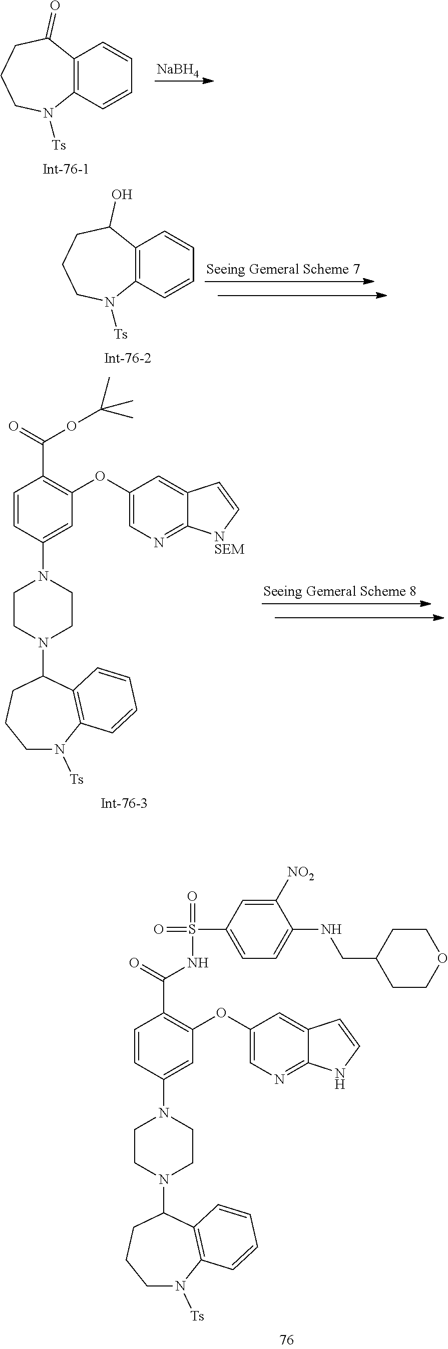

### Step 1: Synthesis of Compound 76-2

To a solution of Int-76-1 (1.0 g, 3.17 mmol, 1.0 eq) in methanol (20 mL) was added NaBH₄ (180 mg, 4.76 mmol, 1.5 eq) at 0° C. and the reaction mixture was stirred at 0° C. for 2 h. H₂O (10 mL) was added to quench the reaction. The solvent was removed under vacuum. H₂O (50 mL) was added and the mixture was extracted with EA (30 mL×3). The combined organic phase was washed with brine, dried over anhydrous Na₂SO₄ and concentrated under vacuum. The crude residue was purified by combine flash (EA/PE=0˜30%) to give Int-76-2 (820 mg, yield 81%). MS(ESI): m/z 300.1 (M-OH).

### Step 2: Synthesis of Compound 76-3

Seeing the preparation of Int-7-15 in General Scheme 7. MS(ESI): m/z 824.3 (M+H+).

### Step 3: Synthesis of Compound 76

Seeing the preparation of Formula 8 in General Scheme 8. MS(ESI): m/z 935.0 (M+H+).

The names and MS (ESI) data of Compound 60 and 76 are shown as follows.

| Compound | | MS(ESI): m/z |
|---|---|---|
| No. | IUPAC Name | (M + H⁺) |
| 60 | 2-((1H-pyrrolo[2,3-b]pyridin-5-yl)oxy)-4- | 858.0 |
| | (4-(3-(methylsulfonyl)-2,3,4,5-tetrahydro- | |
| | 1H-benzo[d]azepin-1-yl)piperazin-1-yl)-N- | |
| | ((3-nitro-4-(((tetrahydro-2H-pyran-4- | |
| | yl)methyl)amino)phenyl)sulfonyl)benzamide | |
| 76 | 2-((1H-pyrrolo[2,3-b]pyridin-5-yl)oxy)-N- | 935.0 |
| | ((3-nitro-4-(((tetrahydro-2H-pyran-4- | |
| | yl)methyl)amino)phenyl)sulfonyl)-4-(4-(1- | |
| | tosyl-2,3,4,5-tetrahydro-1H-benzo[b]azepin- | |
| | 5-yl)piperazin-1-yl)benzamide | |

### Example 29: Synthesis of Compound 105

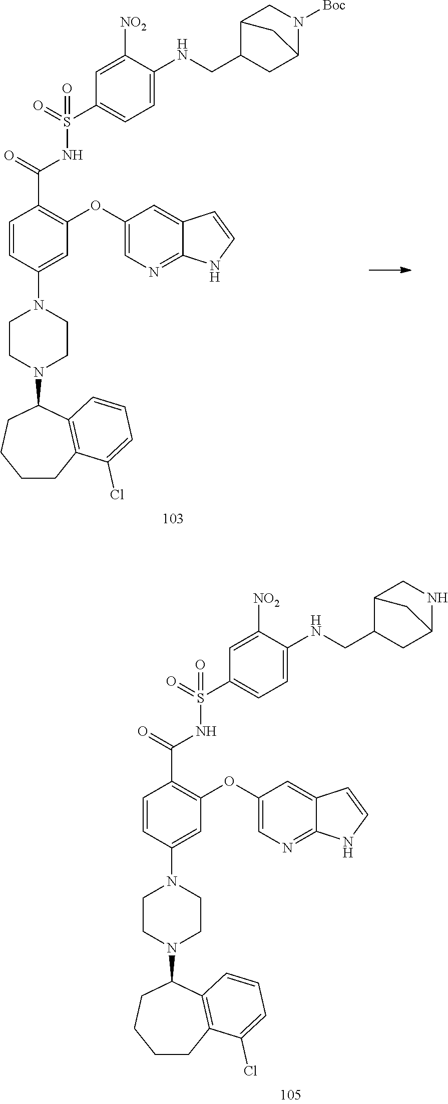 [SMILES: CC(C)(C)OC(=O)N1CC2CC1CC2CNc1ccc(S(=O)(=O)NC(=O)c2ccc(N3CCN([C@@H]4CCCCc5c(Cl)cccc54)CC3)cc2Oc2cnc3[nH]ccc3c2)cc1[N+](=O)[O-] ; O=C(NS(=O)(=O)c1ccc(NCC2CC3CC2CN3)c([N+](=O)[O-])c1)c1ccc(N2CCN([C@@H]3CCCCc4c(Cl)cccc43)CC2)cc1Oc1cnc2[nH]ccc2c1]

To a solution of Compound 103 (37 mg, 0.04 mmol) in DCM (3 mL) was added TFA (1 mL). The reaction mixture was stirred at RT for 1 h. The solvent was removed under vacuum and the crude residue was purified by Prep-HPLC to give the Compound 105 (11.4 mg, yield 35%). MS(ESI): m/z 826.3 (M+H+).

### Example 30: Synthesis of Compound 110

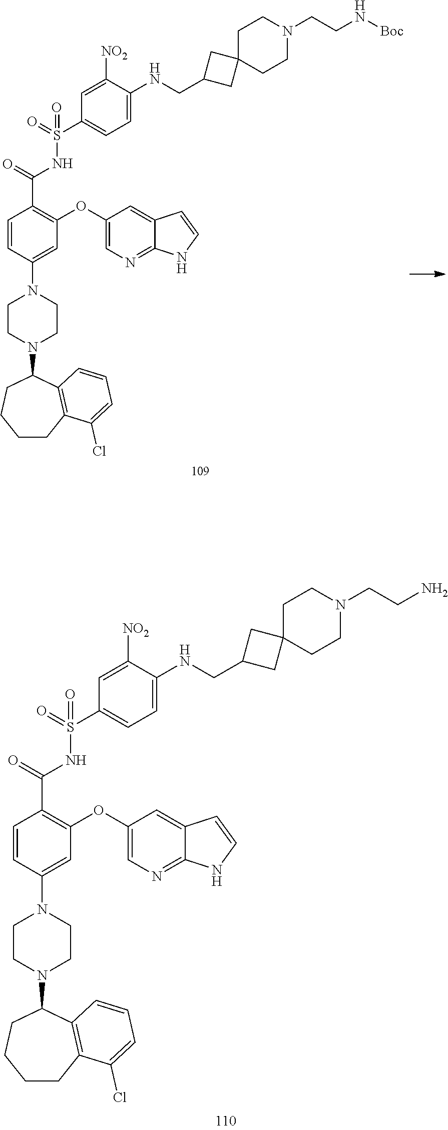 [SMILES: CC(C)(C)OC(=O)NCCN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2 ; NCCN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2]

To a solution of Compound 109 (180 mg, 0.36 mmol) in DCM (10 ml) was added 4M HCL/dioxane (10 ml) and the mixture was stirred for 2 h at RT. LCMS showed reaction was completed. Concentrated and residue was purified by prep-HPLC to give Compound 110 (30 mg, Yield: 18%). MS(ESI): m/z 896.2 (M+H+).

The names and MS (ESI) data of Compound 105 and 110 are shown as follows.

| Compound | | MS(ESI): m/z |
|---|---|---|
| No. | IUPAC Name | (M + H⁺) |
| 105 | 2-((1H-pyrrolo[2,3-b]pyridin-5-yl)oxy)-N-((4-(((2- | 826.3 |
| | azabicyclo[2.2.1]heptan-5-yl)methyl)amino)-3- | |
| | nitrophenyl)sulfonyl)-4-(4-((R)-1-chloro-6,7,8,9- | |
| | tetrahydro-5H-benzo[7]annulen-5-yl)piperazin-1- | |
| | yl)benzamide | |
| 110 | (R)-2-((1H-pyrrolo[2,3-b]pyridin-5-yl)oxy)-N-((4- | 896.2 |
| | (((7-(2-aminoethyl)-7-azaspiro[3.5]nonan-2- | |
| | yl)methyl)amino)-3-nitrophenyl)sulfonyl)-4-(4-(1- | |
| | chloro-6,7,8,9-tetrahydro-5H-benzo[7]annulen-5- | |
| | yl)piperazin-1-yl)benzamide | |

### Example 31: Synthesis of Compound 31B

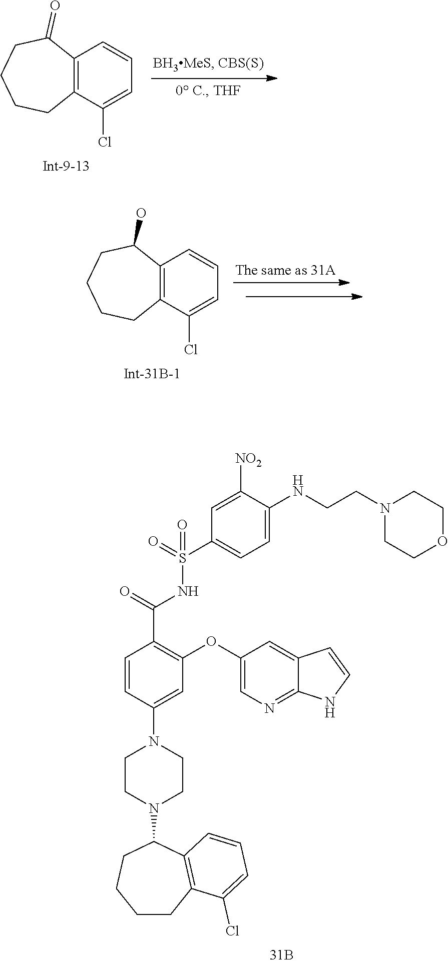 [SMILES: O=C1CCCCc2c(Cl)cccc21 ; O[C@@H]1CCCCc2c(Cl)cccc21 ; O=C(NS(=O)(=O)c1ccc(NCCN2CCOCC2)c([N+](=O)[O-])c1)c1ccc(N2CCN([C@H]3CCCCc4c(Cl)cccc43)CC2)cc1Oc1cnc2[nH]ccc2c1]

### Step 1: Synthesis of Int-31B-1

Using (S)-3,3-Diphenyl-1-methylpyrrolidino[1,2-c]-1,3,2-oxazaborole instead of (R)-3,3-Diphenyl-1-methylpyrrolidino[1,2-c]-1,3,2-oxazaborole, the Int-31B-1 is synthesize as shown as Int-31A-1. MS(ESI): m/z 179.1 (M-OH).

### Step 2: Synthesis of Int-31B

Using Int-31B-1 as starting material, Compound 31B was synthesized as shown as Int-31A. MS(ESI): m/z 829.0 (M+H+).

### Example 32: Synthesis of Compound 18B

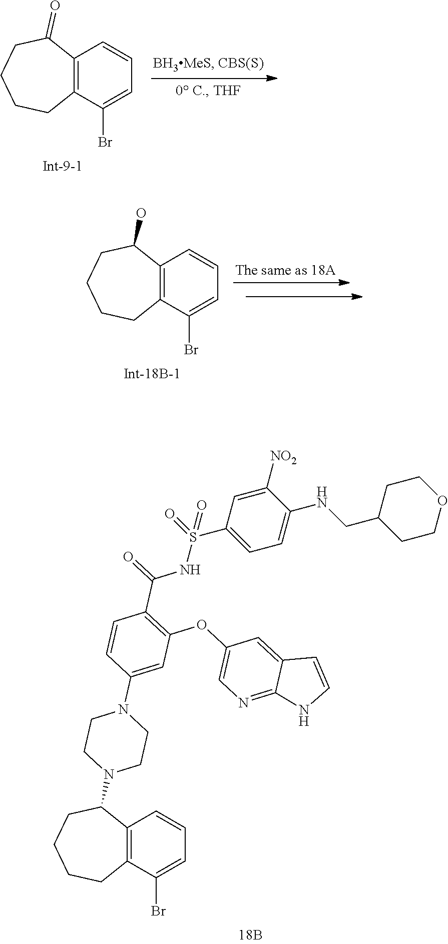 [SMILES: O=C1CCCCc2c(Br)cccc21 ; O[C@@H]1CCCCc2c(Br)cccc21 ; O=C(NS(=O)(=O)c1ccc(NCC2CCOCC2)c([N+](=O)[O-])c1)c1ccc(N2CCN([C@H]3CCCCc4c(Br)cccc43)CC2)cc1Oc1cnc2[nH]ccc2c1]

### Step 1: Synthesis of Int-18B-1

Using (S)-3,3-Diphenyl-1-methylpyrrolidino[1,2-c]-1,3,2-oxazaborole instead of (R)-3,3-Diphenyl-1-methylpyrrolidino[1,2-c]-1,3,2-oxazaborole, Int-18B-1 is synthesize as shown as Int-18A-1. MS(ESI): m/z 223.1 (M-OH).

### Step 2: Synthesis of 18B

Using Int-18B-1 as starting material, the Compound 18B is synthesize as shown as Compound 18A. MS(ESI): m/z 858.0 (M+H+).

| Compound | | MS(ESI): m/z |
|---|---|---|
| No. | IUPAC Name | (M + H⁺) |
| 31B | (S)-2-((1H-pyrrolo[2,3-b]pyridin-5-yl)oxy)-4-(4-(1- | 829.0 |
| | chloro-6,7,8,9-tetrahydro-5H-benzo[7]annulen-5- | |
| | yl)piperazin-1-yl)-N-((4-((2- | |
| | morpholinoethyl)amino)-3- | |
| | nitrophenyl)sulfonyl)benzamide | |
| 18B | (S)-2-((1H-pyrrolo[2,3-b]pyridin-5-yl)oxy)-4-(4-(1- | 858.0 |
| | bromo-6, 7,8,9-tetrahydro-5H-benzo[7]annulen-5- | |
| | yl)piperazin-1-yl)-N-((3-nitro-4-(((tetrahydro-2H- | |
| | pyran-4- | |
| | yl)methyl)amino)phenyl)sulfonyl)benzamide | |

### Example 33: Synthesis of Compound 116 & 117 &118 & 119 & 120 & 24 & 48

### Step 1: Synthesis of Key Intermediate Int-9-21

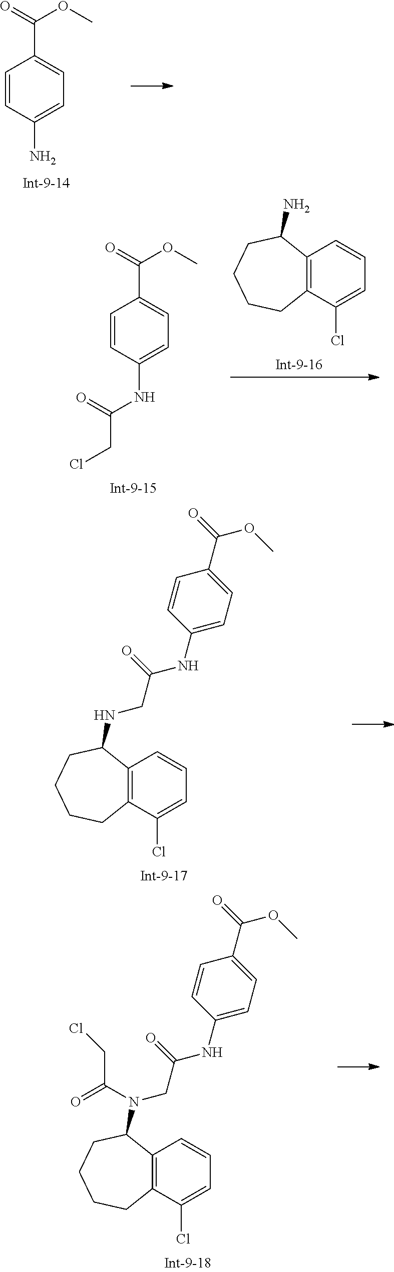

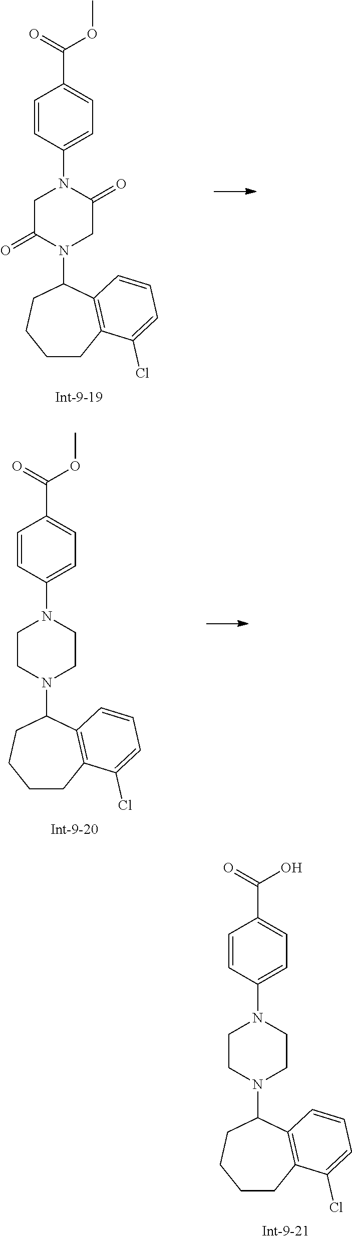 [SMILES: COC(=O)c1ccc(N2CC(=O)N(C3CCCCc4c(Cl)cccc43)CC2=O)cc1 ; COC(=O)c1ccc(N2CCN(C3CCCCc4c(Cl)cccc43)CC2)cc1 ; O=C(O)c1ccc(N2CCN(C3CCCCc4c(Cl)cccc43)CC2)cc1]

### Step 2: Synthesis of Int-9-15

To a stirred solution of Int-9-14 (2.0 g, 13.23 mmol, 1.0 eq) in DCM (50 mL) was added and TEA (9.2 mL, 66.15 mmol, 5.0 eq) and chloroacetyl chloride (4.48 g, 39.69 mmol, 3.0 eq) at 0° C. The resulting mixture was stirred at rt for 2 h and LC-MS shows the total consumption of Int-9-14. It was quench by aqueous NH₄HCO₃ solution and extracted with DCM (3×100 mL). The combined organic layer was washed with brine (1×50 mL), dried over sodium sulfate and concentrated under reduced pressure. The residue was purified by flash chromatography (EA/PE=0˜50%) to give Int-9-15 (1.8 g, yield 60%). MS(ESI): m/z 228.0 (M+H+).

### Step 3: Synthesis of Int-9-17

To a solution of Int-9-15 (1.08 g, 4.74 mmol, 1.1 eq), Int-9-16 (1.0 g, 4.31 mmol, 1.0 eq), NaI (1.94 g, 12.93 mmol, 3.0 eq), K₂CO₃ (3.0 g, 21.55 mmol, 5.0 eq) in acetonitrile (50 mL) was heated at 90° C. for 5 h. H₂O (100 mL) was added and the mixture was extracted with EA (50 mL×3). The combined organic phase was washed with brine, dried over anhydrous Na₂SO₄ and concentrated under vacuum. The crude residue was purified by combine flash (EA/PE=0˜40%) to give Int-9-17 (1.66 g, yield 99%). MS(ESI): m/z 387.0 (M+H+).

### Step 4: Synthesis of Int-9-18

To a solution of Int-9-17 (0.17 g, 0.44 mmol, 1.0 eq) in DCM (50 mL) was added TEA (0.22 g, 2.2 mmol, 5.0 eq) and 2-chloroacetyl chloride (75 mg, 0.66 mmol, 1.5 eq) at 0° C. The reaction mixture was stirred at RT for 1 h. The organic phase was washed with saturated NaHCO₃ (30 mL×1), brine, dried over anhydrous Na₂SO₄ and concentrated under vacuum. The crude residue was purified by combine flash (EA/PE=0˜40%) to give Int-9-18 (150 mg, yield 74%). MS(ESI): m/z 463.3 (M+H+).

### Step 5: Synthesis of Int-9-19

To a solution of Int-9-18 (0.5 g, 1.08 mmol, 1.0 eq), NaI (0.49 g, 3.25 mmol, 3.0 eq), K₂CO₃ (0.49 g, 3.25 mmol, 3.0 eq) in acetonitrile (50 mL) was heated at 90° C. for 5 h. EA (150 mL) was added and the organic phase was washed with H₂O (50 mL×1), brine, dried over anhydrous Na₂SO₄ and concentrated under vacuum to give Int-9-19 (400 mg, yield 87%). MS(ESI): m/z 427.2 (M+H+).

### Step 6: Synthesis of Int-9-20

To a solution of Int-9-19 (1.3 g, 3.05 mmol, 1.0 eq) in THE (50 mL) was added 1M BH3·THF (31 mL, 31 mmol, 10.0 eq) and the mixture was stirred at RT for 2 h. MeOH (20 mL) was added slowly and the mixture was concentrated under vacuum. The crude residue was purified by combine flash (EA/PE=0˜40%) to give Int-9-20 (900 mg, yield 74%). MS(ESI): m/z 399.2 (M+H+).

### Step 7: Synthesis of Int-9-21

To a solution of Int-9-20 (800 mg, 2.01 mmol) in EtOH (50 mL) was added NaOH(aq) (20 mL) and the mixture was stirred at 90° C. for 4 h. LCMS showed OK, concentrated under vacuum, HCl was added to pH=6, the mixture was extracted with EA (50 mL×3). The combined organic phase was wished with brine, dried over anhydrous Na₂SO₄ and concentrated under vacuum. The crude residue was purified by combine flash (EA/PE=0˜50%) to give Int-9-21 (700 mg, yield 91%). MS(ESI): m/z 385.2 (M+H+).

### Step 8: Synthesis of Compound 116

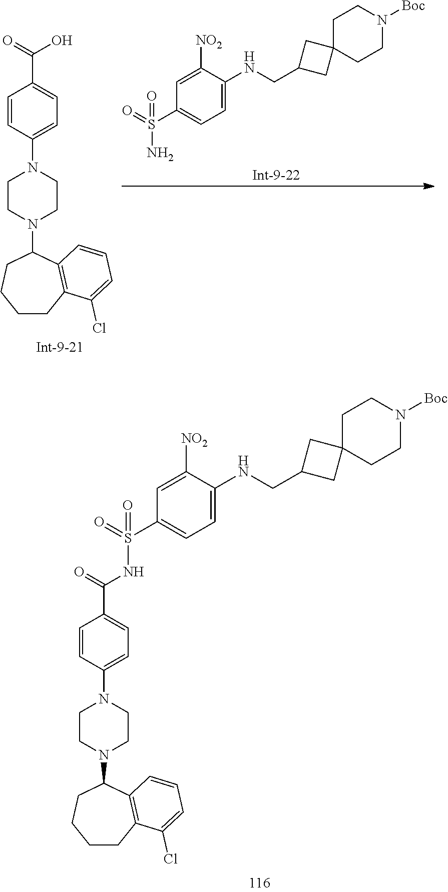 [SMILES: O=C(O)c1ccc(N2CCN(C3CCCCc4c(Cl)cccc43)CC2)cc1 ; CC(C)(C)OC(=O)N1CCC2(CC1)CC(CNc1ccc(S(N)(=O)=O)cc1[N+](=O)[O-])C2 ; CC(C)(C)OC(=O)N1CCC2(CC1)CC(CNc1ccc(S(=O)(=O)NC(=O)c3ccc(N4CCN([C@@H]5CCCCc6c(Cl)cccc65)CC4)cc3)cc1[N+](=O)[O-])C2]

To a solution of Int-9-21 (100 mg, 0.26 mmol, 1.0 eq) and Int-9-22 (142 mg, 0.31 mmol, 1.2 eq) in anhydrous DMF (2 mL) was added EDCI (75 mg, 0.39 mmol, 1.5 eq), DMAP (246 mg, 1.30 mmol, 5.0 eq) and DIEA (168 mg, 1.30 mmol, 5.0 eq) at rt. The reaction mixture was stirred at 30° C. for 16 h under N₂ atmosphere. LCMS showed the reaction was completed. The reaction mixture was poured into EtOAc (30 mL) and washed with H₂O (2×25 mL). The extract was washed with brine (1×20 mL), dried over Na₂SO₄ and concentrated. The crude product was purified by prep-TLC (DCM:MeOH=10:1, at 254 nm) to give Compound 116 (23 mg, yield 11%). MS(ESI): m/z 821.3 (M+H+).

### Step 9: Synthesis of Compound 117

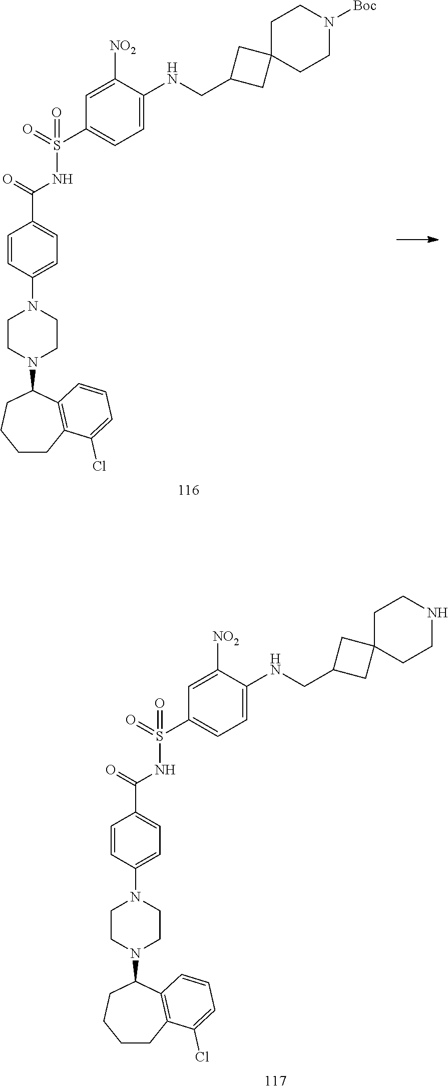 [SMILES: CC(C)(C)OC(=O)N1CCC2(CC1)CC(CNc1ccc(S(=O)(=O)NC(=O)c3ccc(N4CCN([C@@H]5CCCCc6c(Cl)cccc65)CC4)cc3)cc1[N+](=O)[O-])C2 ; O=C(NS(=O)(=O)c1ccc(NCC2CC3(CCNCC3)C2)c([N+](=O)[O-])c1)c1ccc(N2CCN([C@@H]3CCCCc4c(Cl)cccc43)CC2)cc1]

To a solution of Compound 116 (50 mg, 0.60 mmol) in anhydrous DCM (3 mL) was added TFA (1 ml). The reaction mixture was stirred at rt for 4 h under N₂ atmosphere. LCMS showed the reaction was completed. The reaction mixture was concentrated and the crude product was purified by prep-TLC (DCM:MeOH=10:1, at 254 nm) to Compound 117 (25 mg, yield 57%). MS(ESI): m/z 721.2 (M+H+).

### Step 10: Synthesis of Compound 118

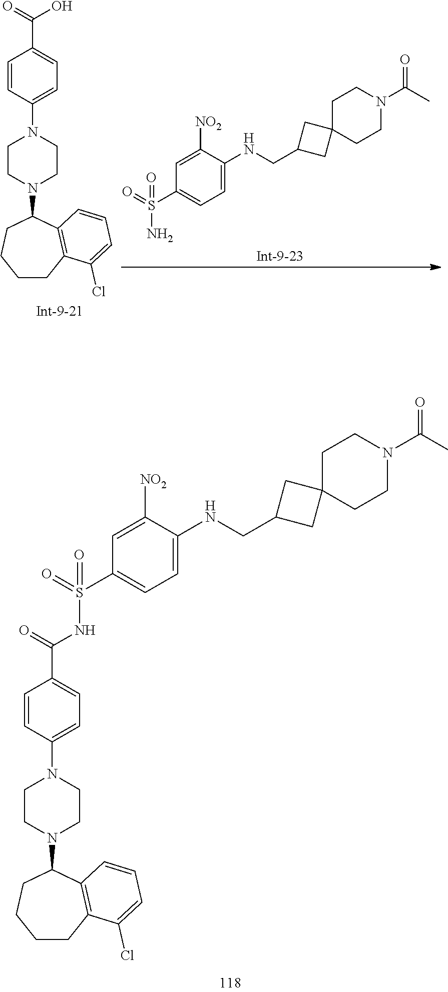

To a solution of Int-9-21 (80 mg, 0.21 mmol, 1.0 eq) and Int-9-23 (99 mg, 0.25 mmol, 1.2 eq) in anhydrous DMSO (2 mL) was added EDCI (60 mg, 0.32 mmol, 1.5 eq), DMAP (198 mg, 1.05 mmol, 5.0 eq) and DIEA (135 mg, 1.05 mmol, 5.0 eq) at rt. The reaction mixture was stirred at 30° C. for 16 h under N₂ atmosphere. LCMS showed the reaction was completed. The reaction mixture was poured into EtOAc (30 mL) and washed with H₂O (2×25 mL). The extract was washed with brine (1×20 mL), dried over Na₂SO₄, concentrated. The crude product was purified by prep-TLC (DCM:MeOH=10:1, at 254 nm) to give Compound 118 (42 mg, yield 26%). MS(ESI): m/z 763.2 (M+H+).

### Step 11: Synthesis of Compound 119

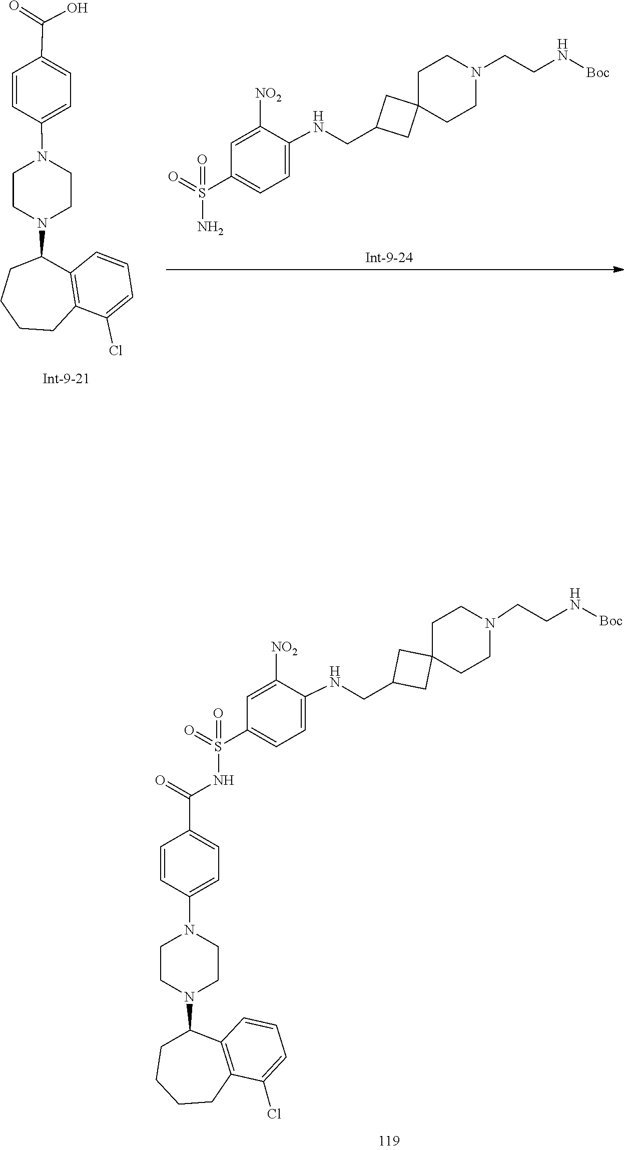 [SMILES: O=C(O)c1ccc(N2CCN([C@@H]3CCCCc4c(Cl)cccc43)CC2)cc1 ; CC(C)(C)OC(=O)NCCN1CCC2(CC1)CC(CNc1ccc(S(N)(=O)=O)cc1[N+](=O)[O-])C2 ; CC(C)(C)OC(=O)NCCN1CCC2(CC1)CC(CNc1ccc(S(=O)(=O)NC(=O)c3ccc(N4CCN([C@@H]5CCCCc6c(Cl)cccc65)CC4)cc3)cc1[N+](=O)[O-])C2]

To a solution of Int-9-21 (200 mg, 0.64 mmol, 1.0 eq) and Int-9-24 (356 mg, 0.71 mmol, 1.1 eq) in anhydrous DMSO (5 mL) was added EDCI (186 mg, 0.98 mmol, 1.5 eq), DMAP (614 mg, 3.25 mmol, 5.0 eq) and DIEA (419 mg, 3.25 mmol, 5.0 eq) at rt. The reaction mixture was stirred at 30° C. for 16 h under N₂ atmosphere. LCMS showed the reaction was completed. The reaction mixture was poured into EtOAc (30 mL) and washed with H₂O (2×25 mL). The extract was washed with brine (1×20 mL), dried over Na₂SO₄. The crude product was purified by prep-TLC (DCM:MeOH=10:1, at 254 nm) to give Compound 119 (100 mg, yield 22%). MS(ESI): m/z 864.3 (M+H+).

### Step 12: Synthesis of Compound 120

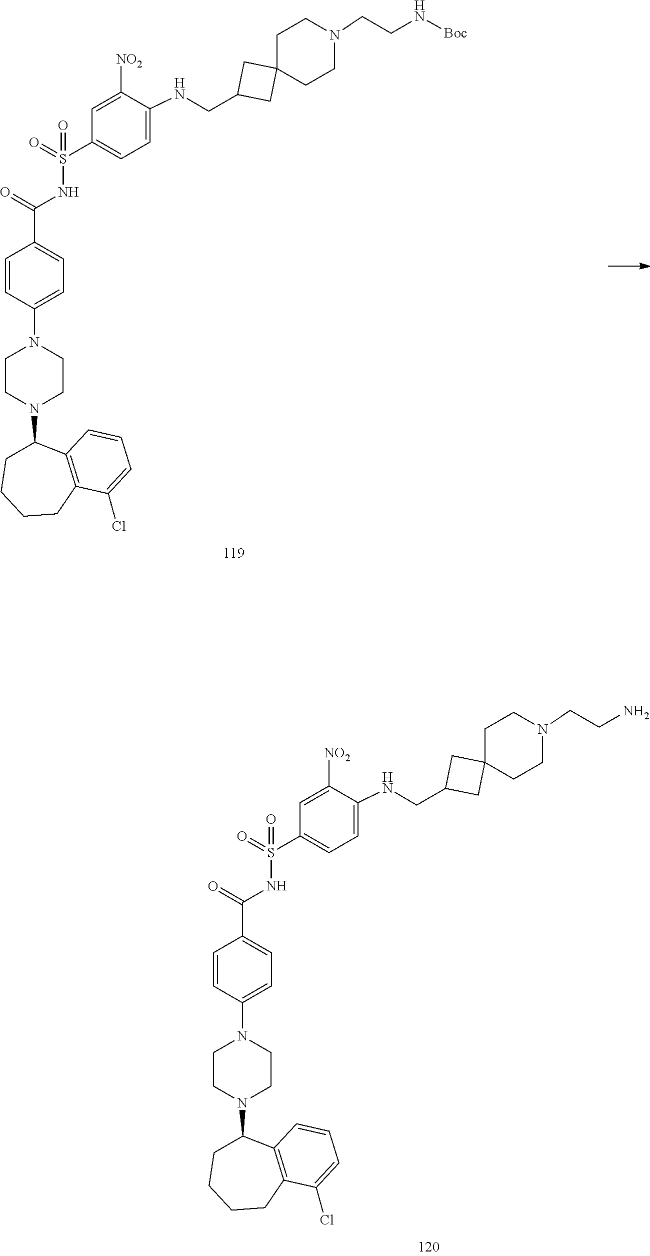 [SMILES: CC(C)(C)OC(=O)NCCN1CCC2(CC1)CC(CNc1ccc(S(=O)(=O)NC(=O)c3ccc(N4CCN([C@@H]5CCCCc6c(Cl)cccc65)CC4)cc3)cc1[N+](=O)[O-])C2 ; NCCN1CCC2(CC1)CC(CNc1ccc(S(=O)(=O)NC(=O)c3ccc(N4CCN([C@@H]5CCCCc6c(Cl)cccc65)CC4)cc3)cc1[N+](=O)[O-])C2]

To a solution of Compound 119 (110 mg, 0.13 mmol) in anhydrous DCM (10 mL) was TFA (3 ml). The mixture was stirred at rt for 6 h under N₂ atmosphere. LCMS showed the reaction was completed. The reaction mixture was concentrated and the crude product was purified by prep-TLC (DCM:MeOH=10:1, at 254 nm) to give Compound 120 (60 mg, yield 62%). MS(ESI): m/z 764.2 (M+H+).

### Step 13: Synthesis of Compound 24

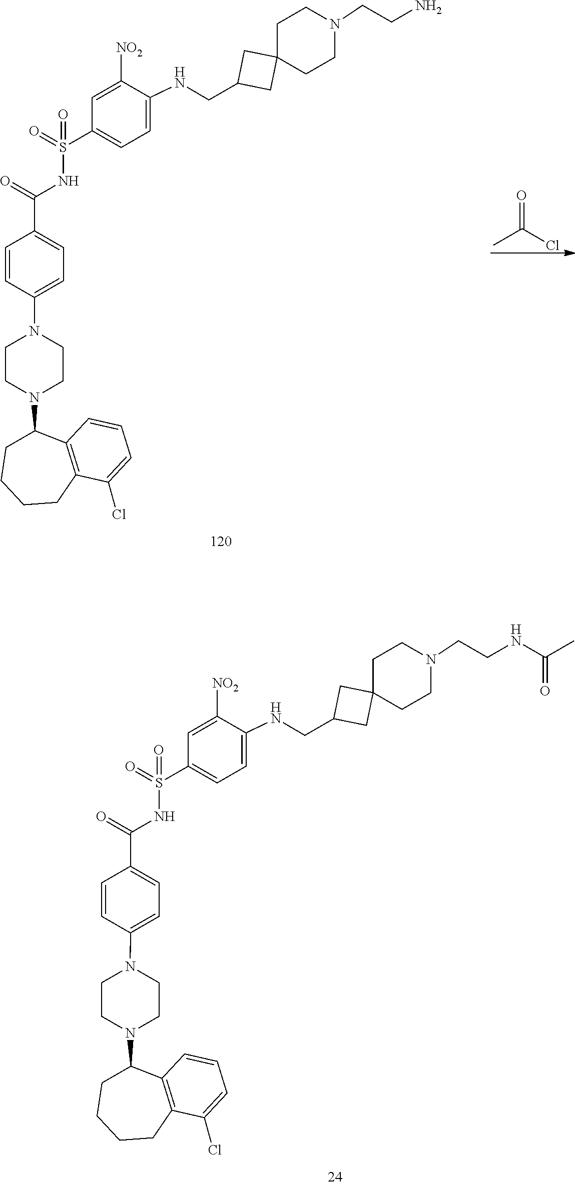

To a solution of Compound 120 (60 mg, 0.078 mmol, 1.0 eq) in anhydrous DMF (5 mL) was added DIEA (30 mg, 0.24 mmol, 3.0 eq) and acetyl chloride (6 mg, 0.078 mmol, 1.0 eq) at rt. The reaction mixture was stirred at rt for 16 h under N₂ atmosphere. LCMS showed the reaction was completed. The reaction mixture was poured into EtOAc (30 mL) and washed with H₂O (2×25 mL). The extract was washed with brine (1×20 mL), dried over Na₂SO₄ and concentrated to give crude product. The crude product was purified by prep-TLC (DCM:MeOH=20:1, at 254 nm) to give Compound 24 (40 mg, 63%). MS(ESI): m/z 806.3 (M+H+).

### Step 14: Synthesis of Compound 48

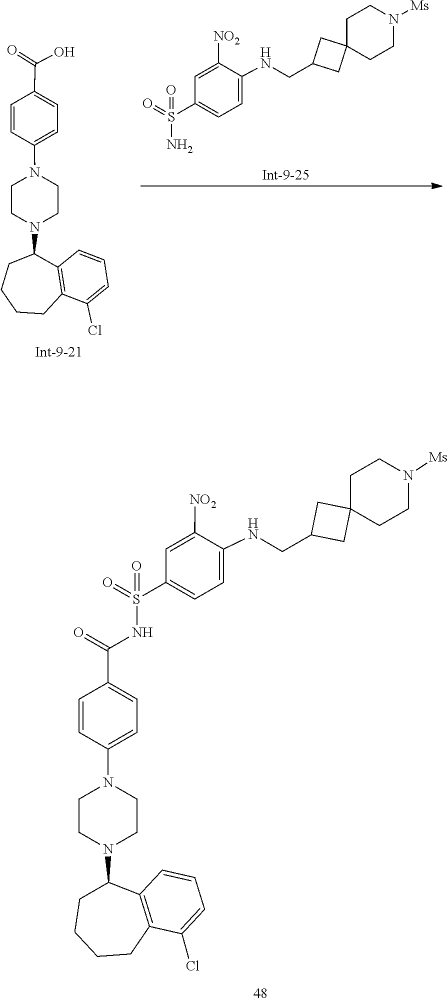 [SMILES: O=C(O)c1ccc(N2CCN([C@@H]3CCCCc4c(Cl)cccc43)CC2)cc1 ; CS(=O)(=O)N1CCC2(CC1)CC(CNc1ccc(S(N)(=O)=O)cc1[N+](=O)[O-])C2 ; CS(=O)(=O)N1CCC2(CC1)CC(CNc1ccc(S(=O)(=O)NC(=O)c3ccc(N4CCN([C@@H]5CCCCc6c(Cl)cccc65)CC4)cc3)cc1[N+](=O)[O-])C2]

To a solution of Int-9-21 (100 mg, 0.26 mmol, 1.0 eq) and Int-9-25 (123 mg, 0.29 mmol, 1.1 eq) in anhydrous DMSO (5 mL) was added EDCI (75 mg, 0.39 mmol, 1.5 eq), DMAP (246 mg, 1.30 mmol, 5.0 eq) and DIEA (168 mg, 1.30 mmol, 5.0 eq) at rt. The reaction mixture was stirred at 30° C. for 16 h under N₂ atmosphere. LCMS showed the reaction was completed. The reaction mixture was poured into EtOAc (30 mL) and washed with H₂O (2×25 mL). The extract was washed with brine (1×20 mL), dried over Na₂SO₄ and concentrated to give crude product. The crude product was purified by prep-TLC (DCM:MeOH=10:1, at 254 nm) to give Compound 48 (60 mg, yield 29%). MS(ESI): m/z 799.0 (M+H+).

The names and MS (ESI) data of Compound 116, 117, 118, 119, 120, 24 and 48 are shown as follows.

| Compound | | MS(ESI): m/z |
|---|---|---|
| No. | IUPAC Name | (M + H⁺) |
| 116 | tert-butyl (R)-2-(((4-(N-(4-(4-(1-chloro-6,7,8,9- | 821.3 |
| | tetrahydro-5H-benzo[7]annulen-5-yl)piperazin- | |
| | 1-yl)benzoyl)sulfamoyl)-2- | |
| | nitrophenyl)amino)methyl)-7- | |
| | azaspiro[3.5]nonane-7-carboxylate | |
| 117 | (R)-N-((4-(((7-azaspiro[3.5]nonan-2- | 721.2 |
| | yl)methyl)amino)-3-nitrophenyl)sulfonyl)-4-(4- | |
| | (1-chloro-6,7,8,9-tetrahydro-5H- | |
| | benzo[7]annulen-5-yl)piperazin-1-yl)benzamide | |
| 118 | (R)-N-((4-(((7-acetyl-7-azaspiro[3.5]nonan-2- | 763.2 |
| | yl)methyl)amino)-3-nitrophenyl)sulfonyl)-4-(4- | |
| | (1-chloro-6,7,8,9-tetrahydro-5H- | |
| | benzo[7]annulen-5-yl)piperazin-1-yl)benzamide | |
| 119 | tert-butyl (R)-(2-(2-(((4-(N-(4-(4-(1-chloro- | 864.3 |
| | 6,7,8,9-tetrahydro-5H-benzo[7]annulen-5- | |
| | yl)piperazin-1-yl)benzoyl)sulfamoyl)-2- | |
| | nitrophenyl)amino)methyl)-7- | |
| | azaspiro[3.5]nonan-7-yl)ethyl)carbamate | |
| 120 | (R)-N-((4-(((7-(2-aminoethyl)-7- | 764.2 |
| | azaspiro[3.5]nonan-2-yl)methyl)amino)-3- | |
| | nitrophenyl)sulfonyl)-4-(4-(1-chloro-6,7,8,9- | |
| | tetrahydro-5H-benzo[7]annulen-5-yl)piperazin- | |
| | 1-yl)benzamide | |
| 24 | (R)-N-((4-(((7-(2-acetamidoethyl)-7- | 806.3 |
| | azaspiro[3.5]nonan-2-yl)methyl)amino)-3- | |
| | nitrophenyl)sulfonyl)-4-(4-(1-chloro-6,7,8,9- | |
| | tetrahydro-5H-benzo[7]annulen-5-yl)piperazin- | |
| | 1-yl)benzamide | |
| 48 | (R)-4-(4-(1-chloro-6,7,8,9-tetrahydro-5H- | 799.0 |
| | benzo[7]annulen-5-yl)piperazin-1-yl)-N-((4-(((7- | |
| | (methylsulfonyl)-7-azaspiro[3.5]nonan-2- | |
| | yl)methyl)amino)-3- | |
| | nitrophenyl)sulfonyl)benzamide | |

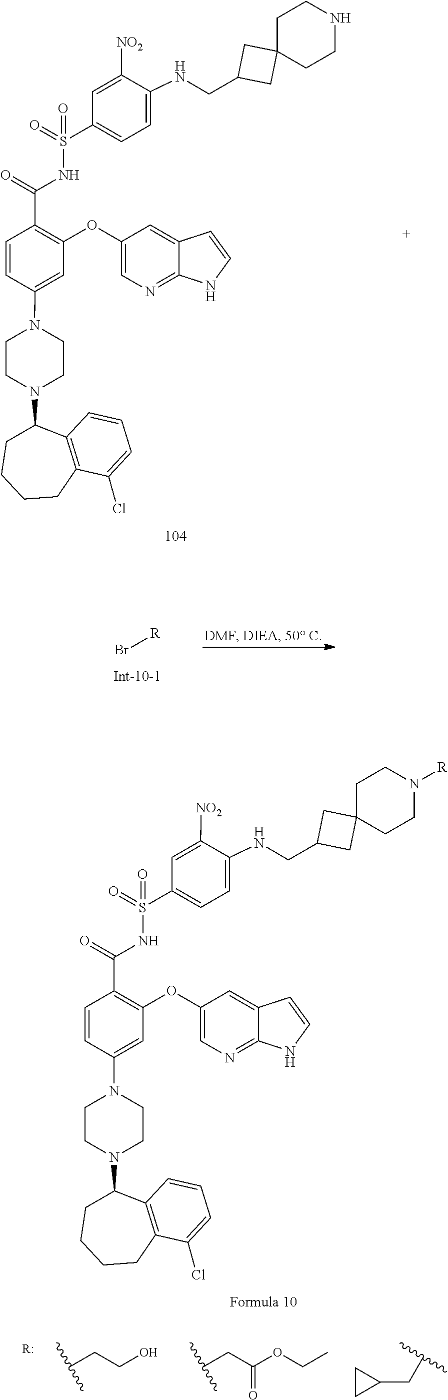

### General Step: Synthesis of Formula 10

To a solution of Compound 104 (1 mmol, 1.0 eq), Int-10-1 (1 mmol, 1 eq) in DMF (10 mL) was added DIEA (2 mmol, 2 eq). The reaction mixture was stirred at 50° C. TLC showed reaction was completed. DCM was added and the organic phase was washed with brine (3×50 mL), dried over sodium sulfate and concentrated under reduced pressure. The residue was purified by flash chromatography (ME/DCM=0˜10%) to give Formula 10.

### Example 34: Synthesis of Compound 121

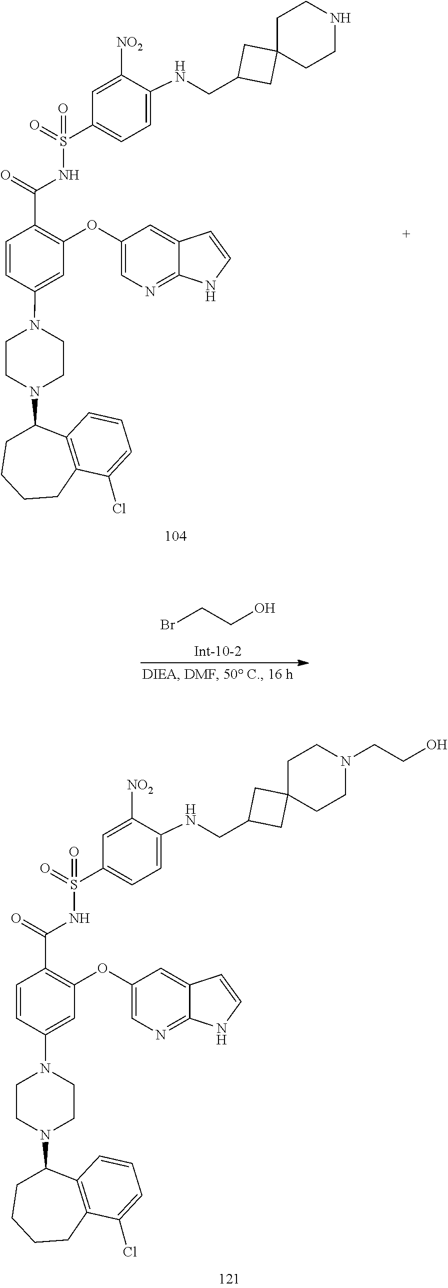

To a solution of Compound 104 (50 mg, 0.06 mmol, 1.0 eq), Int-10-2 (7.5 mg, 0.06 mmol, 1 eq) in DMF (2 mL) was added DIEA (15 mg, 0.12 mmol, 2 eq). The reaction mixture was stirred at 50° C. for 16 h. TLC showed reaction was completed. DCM (50 mL) was added and the organic phase was washed with brine (3×20 mL), dried over sodium sulfate and concentrated under reduced pressure. The residue was purified by flash chromatography (ME/DCM=0˜10%) to give Compound 121 (15 mg, 28% yield). MS(ESI): m/z 896.3 (M+H⁺).

The following compounds were prepared according to the above described methods using different starting materials.

| Compound | | MS(ESI): m/z |
|---|---|---|
| ID | IUPAC Name | (M + H⁺) |
| 122 | ethyl (R)-2-(2-(((4-(N-(2-((1H-pyrrolo[2,3- | 939.3 |
| | b]pyridin-5-yl)oxy)-4-(4-(1-chloro-6,7,8,9- | |
| | tetrahydro-5H-benzo[7]annulen-5-yl)piperazin- | |
| | 1-yl)benzoyl)sulfamoyl)-2- | |
| | nitrophenyl)amino)methyl)-7- | |
| | azaspiro[3.5]nonan-7-yl)acetate | |
| 129 | (R)-2-((1H-pyrrolo[2,3-b]pyridin-5-yl)oxy)-4-(4- | 907.2 |
| | (1-chloro-6,7,8,9-tetrahydro-5H- | |
| | benzo[7]annulen-5-yl)piperazin-1-yl)-N-((4-(((7- | |
| | (cyclopropylmethyl)-7-azaspiro[3.5]nonan-2- | |
| | yl)methyl)amino)-3- | |
| | nitrophenyl)sulfonyl)benzamide | |

### Example 35: Synthesis of Compound 123

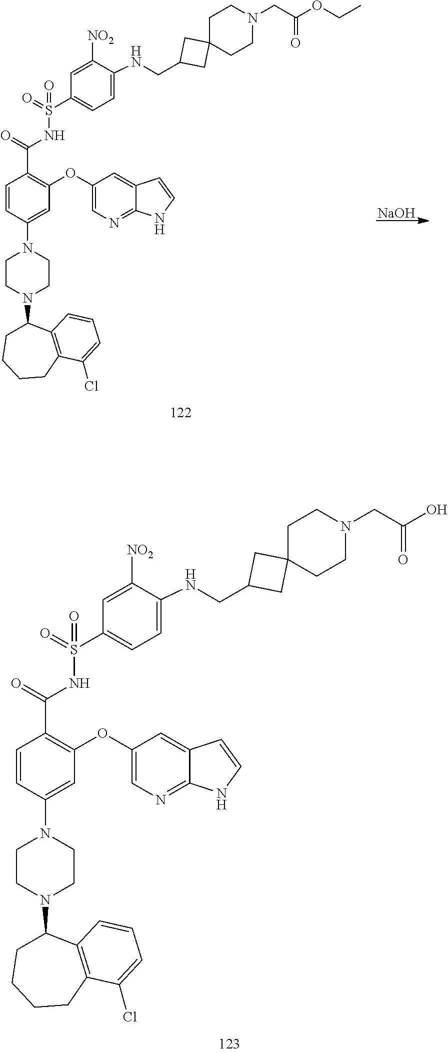 [SMILES: CCOC(=O)CN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2 ; "NaOH" ; O=C(O)CN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2]

A solution of Compound 122 (80 mg, 0.09 mmol, 1 eq), 10% NaOH (2 mL) in ethanol (150 mL) was stirred at rt for 3 h. TLC showed reaction was completed. Ethanol was removed and water (50 mL) was added. Then 1M HCl aqueous solution was added to adjust the pH to 6-7 and the mixture was extracted with DCM (50 mL×3). The combined organic phase was washed with brine, dried over anhydrous Na₂SO₄ and concentrated. The crude mixture was purified by flash chromatography (ME/DCM=0˜10%) to give Compound 123 (60 mg, 73% yield). MS(ESI): m/z 911.0 (M+H⁺).

### Example 36: Synthesis of Compound 124

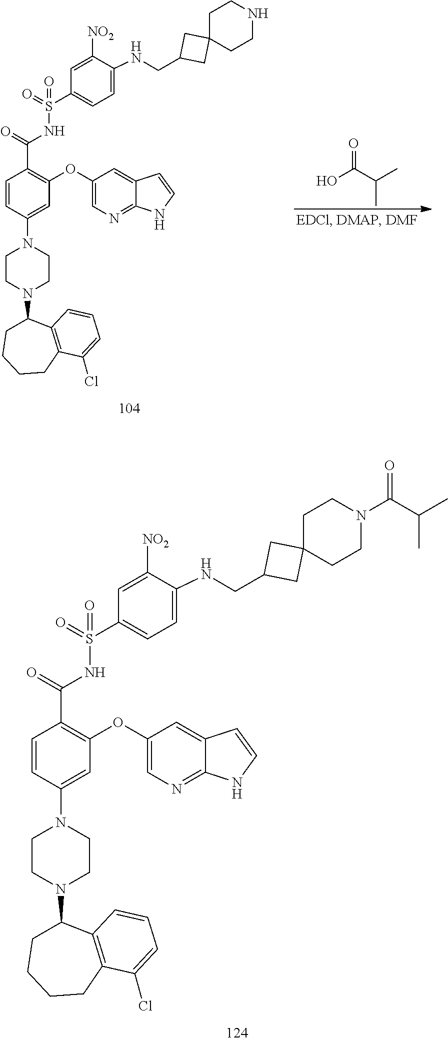

To a stirred solution of Compound 104 (100 mg, 0.12 mmol, 1.0 eq) in anhydrous DMF (2 ml) was added isobutyric acid (11 mg, 0.12 mmol, 1.0 eq), EDCI (30 mg, 0.16 mmol, 1.3 eq), DMAP (43 mg, 0.36 mmol, 3.0 eq), DIPEA (46 mg, 0.36 mmol, 3.0 eq). The mixture was stirred at RT for 16 h. The reaction mixture was concentrated, and the residue was dissolved in DCM (100 mL). The organic layer was washed with water (30 mL×3), dried over anhydrous Na₂SO₄ and evaporated in vacuum. The residue was purified by flash chromatography (ME/DCM=0˜10%) to give Compound 124 (30 mg, yield 29%). MS(ESI): m/z 923.3 (M+H⁺).

### Example 37: Synthesis of Compound 125

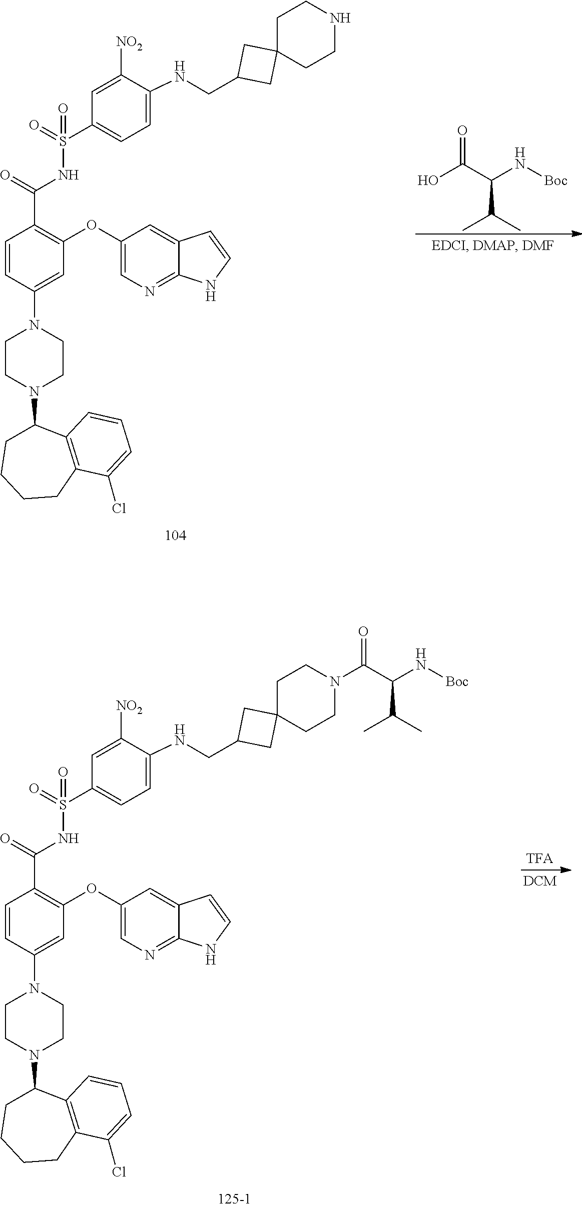 [SMILES: O=C(NS(=O)(=O)c1ccc(NCC2CC3(CCNCC3)C2)c([N+](=O)[O-])c1)c1ccc(N2CCN([C@@H]3CCCCc4c(Cl)cccc43)CC2)cc1Oc1cnc2[nH]ccc2c1 ; CC(C)[C@H](NC(=O)OC(C)(C)C)C(=O)O ; CC(C)[C@H](NC(=O)OC(C)(C)C)C(=O)N1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2]

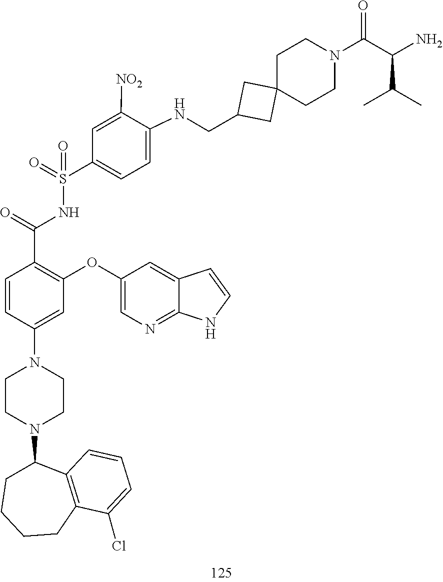 [SMILES: CC(C)[C@H](N)C(=O)N1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2]

### Step 2: Synthesis of Compound 125

To a stirred solution of Compound 104 (100 mg, 0.12 mmol, 1.0 eq) in anhydrous DMF (2 ml) was added (tert-butoxycarbonyl)-L-valine (26 mg, 0.12 mmol, 1.0 eq), EDCI (30 mg, 0.16 mmol, 1.3 eq), DMAP (43 mg, 0.36 mmol, 3.0 eq), DIPEA (46 mg, 0.36 mmol, 3.0 eq). The mixture was stirred at RT for 16 h. The reaction mixture was concentrated, and the residue was dissolved in DCM (100 mL). The organic layer was washed with water (30 mL×3), dried over anhydrous Na₂SO₄ and evaporated in vacuum. The residue was purified by flash chromatography (ME/DCM=0˜10%) to give Compound 125-1 (70 mg, yield 56%). MS(ESI): m/z 1052.4 (M+H⁺).

### Step 2: Synthesis of Compound 125

A solution of Compound 125-1 (70 mg, 0.07 mmol, 1 eq), CFA (2 mL) in DCM (20 mL) was stirred at rt for 3 h. TLC showed reaction was completed. DCM (100 mL) was added, it was washed with brine, dried over anhydrous Na₂SO₄ and concentrated. The crude mixture was purified by flash chromatography (ME/DCM=0˜10%) to give Compound 125 (45 mg, 72% yield). MS(ESI): m/z 952.3 (M+H⁺).

### Example 38: Synthesis of Compound 130 and Compound 126

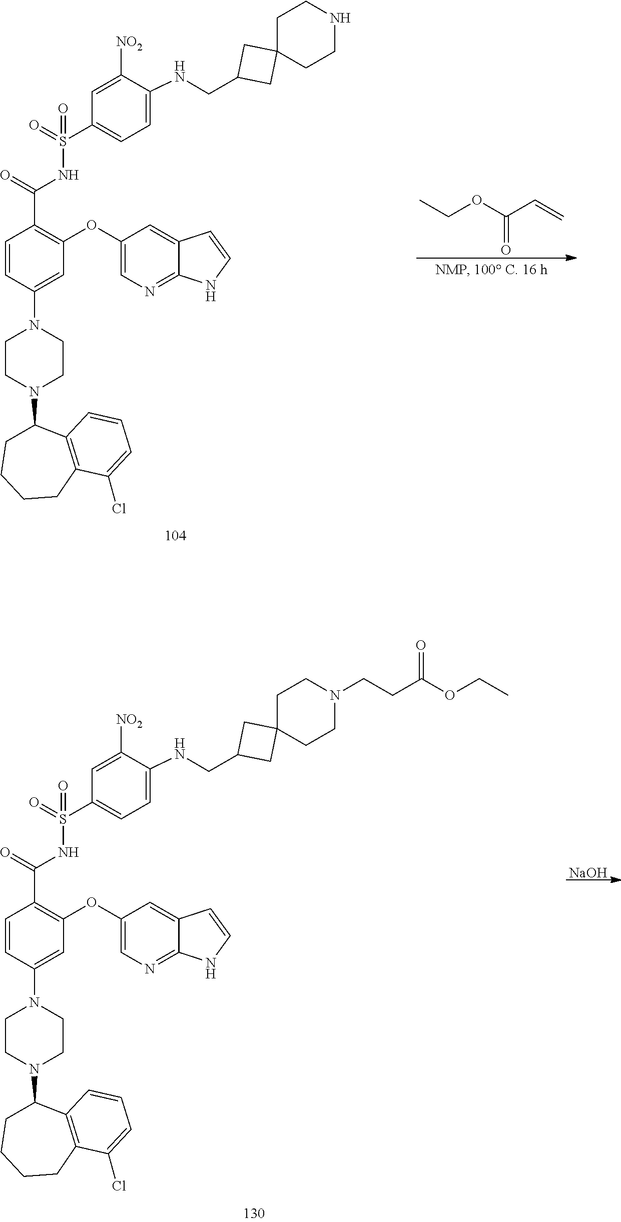

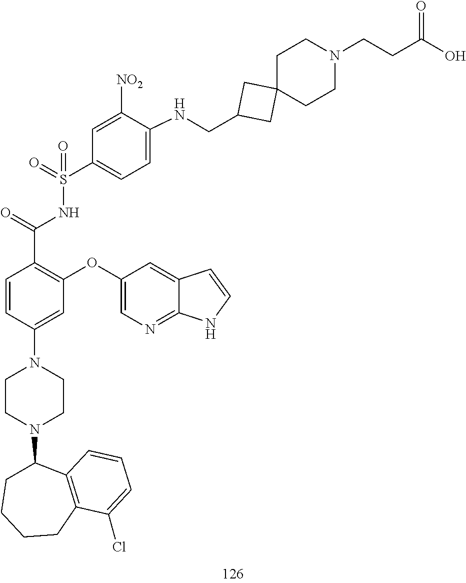 [SMILES: O=C(O)CCN1CCC2(CC1)CC(CNc1ccc(S(=O)(=O)NC(=O)c3ccc(N4CCN([C@@H]5CCCCc6c(Cl)cccc65)CC4)cc3Oc3cnc4[nH]ccc4c3)cc1[N+](=O)[O-])C2]

### Step 1: Synthesis of Compound 130

A solution of Compound 104 (100 mg, 0.12 mmol, 1 eq), ethyl acrylate (15 mg, 0.14 mmol, 1.2 eq) in NMP (10 mL) was stirred at 100° C. for 16 h. TLC showed reaction was completed. DCM (100 mL) was added, it was washed with brine, dried over anhydrous Na₂SO₄ and concentrated. The crude mixture was purified by flash chromatography (ME/DCM=0˜10%) to give Compound 130 (75 mg, 65% yield). MS(ESI): m/z 953.3 (M+H⁺).

### Step 2: Synthesis of Compound 126

A solution of Compound 130 (60 mg, 0.06 mmol, 1 eq), 10% NaOH (2 mL) in ethanol (150 mL) was stirred at rt for 3 h. TLC showed reaction was completed. Ethanol was removed and water (50 mL) was added. Then 1M HCl aqueous solution was added to adjust the pH to 6-7 and the mixture was extracted with DCM (50 mL×3). The combined organic phase was washed with brine, dried over anhydrous Na₂SO₄ and concentrated. The crude mixture was purified by flash chromatography (ME/DCM=0˜10%) to give Compound 126 (38 mg, 68% yield). MS(ESI): m/z 925.7 (M+H⁺).

### Example 39: Synthesis of Compound 131

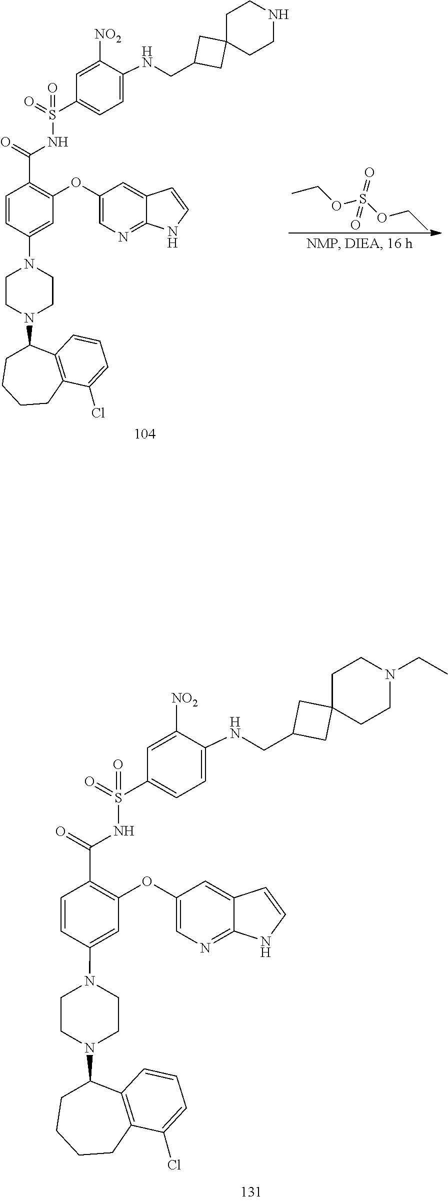

To a solution of Compound 104 (100 mg, 0.12 mmol, 1.0 eq), DIEA (31 mg, 0.24 mmol, 2 eq) in NMP (5 mL) was added compound diethyl sulfate (22 mg, 0.14 mmol, 1.2 eq). The reaction mixture was stirred at RT for 16 h. TLC showed reaction was completed. DCM was added and the organic phase was washed with brine (3×50 mL), dried over sodium sulfate and concentrated under reduced pressure. The residue was purified by flash chromatography (ME/DCM=0˜10%) to give Compound 131 (42 mg, 40% yield). MS(ESI): m/z 881.2 (M+H⁺).

| Compound | | MS(ESI): m/z |
|---|---|---|
| ID | IUPAC Name | (M + H⁺) |
| 121 | (R)-2-((1H-pyrrolo[2,3-b]pyridin-5-yl)oxy)-4-(4-(1- | 896.3 |
| | chloro-6,7,8,9-tetrahydro-5H-benzo[7]annulen-5- | |
| | yl)piperazin-1-yl)-N-((4-(((7-(2-hydroxyethyl)-7- | |
| | azaspiro[3.5]nonan-2-yl)methyl)amino)-3- | |
| | nitrophenyl)sulfonyl)benzamide | |
| 122 | ethyl (R)-2-(2-(((4-(N-(2-((1H-pyrrolo[2,3-b]pyridin- | 939.3 |
| | 5-yl)oxy)-4-(4-(1-chloro-6,7,8,9-tetrahydro-5H- | |
| | benzo[7]annulen-5-yl)piperazin-1- | |
| | yl)benzoyl)sulfamoyl)-2-nitrophenyl)amino)methyl)- | |
| | 7-azaspiro[3.5]nonan-7-yl)acetate | |
| 123 | (R)-2-(2-(((4-(N-(2-((1H-pyrrolo[2,3-b]pyridin-5- | 911.0 |
| | yl)oxy)-4-(4-(1-chloro-6,7,8,9-tetrahydro-5H- | |
| | benzo[7]annulen-5-yl)piperazin-1- | |
| | yl)benzoyl)sulfamoyl)-2-nitrophenyl)amino)methyl)- | |
| | 7-azaspiro[3.5]nonan-7-yl)acetic acid | |
| 124 | (R)-2-((1H-pyrrolo[2,3-b]pyridin-5-yl)oxy)-4-(4-(1- | 923.3 |
| | chloro-6,7,8,9-tetrahydro-5H-benzo[7]annulen-5- | |
| | yl)piperazin-1-yl)-N-((4-(((7-isobutyryl-7- | |
| | azaspiro[3.5]nonan-2-yl)methyl)amino)-3- | |
| | nitrophenyl)sulfonyl)benzamide | |
| 125 | 2-((1H-pyrrolo[2,3-b]pyridin-5-yl)oxy)-N-((4-(((7- | 952.3 |
| | (L-valyl)-7-azaspiro[3.5]nonan-2-yl)methyl)amino)- | |
| | 3-nitrophenyl)sulfonyl)-4-(4-((R)-1-chloro-6,7,8,9- | |
| | tetrahydro-5H-benzo[7]annulen-5-yl)piperazin-1- | |
| | yl)benzamide | |
| 126 | (R)-3-(2-(((4-(N-(2-((1H-pyrrolo[2,3-b]pyridin-5- | 925.7 |
| | yl)oxy)-4-(4-(1-chloro-6,7,8,9-tetrahydro-5H- | |
| | benzo[7]annulen-5-yl)piperazin-1- | |
| | yl)benzoyl)sulfamoyl)-2-nitrophenyl)amino)methyl)- | |
| | 7-azaspiro[3.5]nonan-7-yl)propanoic acid | |
| 129 | (R)-2-((1H-pyrrolo[2,3-b]pyridin-5-yl)oxy)-4-(4-(1- | 907.2 |
| | chloro-6,7,8,9-tetrahydro-5H-benzo[7]annulen-5- | |
| | yl)piperazin-1-yl)-N-((4-(((7-(cyclopropylmethyl)-7- | |
| | azaspiro[3.5]nonan-2-yl)methyl)amino)-3- | |
| | nitrophenyl)sulfonyl)benzamide | |
| 130 | ethyl (R)-3-(2-(((4-(N-(2-((1H-pyrrolo[2,3-b]pyridin- | 953.3 |
| | 5-yl)oxy)-4-(4-(1-chloro-6,7,8,9-tetrahydro-5H- | |
| | benzo[7]annulen-5-yl)piperazin-1- | |
| | yl)benzoyl)sulfamoyl)-2-nitrophenyl)amino)methyl)- | |
| | 7-azaspiro[3.5]nonan-7-yl)propanoate | |
| 131 | (R)-2-((1H-pyrrolo[2,3-b]pyridin-5-yl)oxy)-4-(4-(1- | 881.2 |
| | chloro-6,7,8,9-tetrahydro-5H-benzo[7]annulen-5- | |
| | yl)piperazin-1-yl)-N-((4-(((7-ethyl-7- | |
| | azaspiro[3.5]nonan-2-yl)methyl)amino)-3- | |
| | nitrophenyl)sulfonyl)benzamide | |

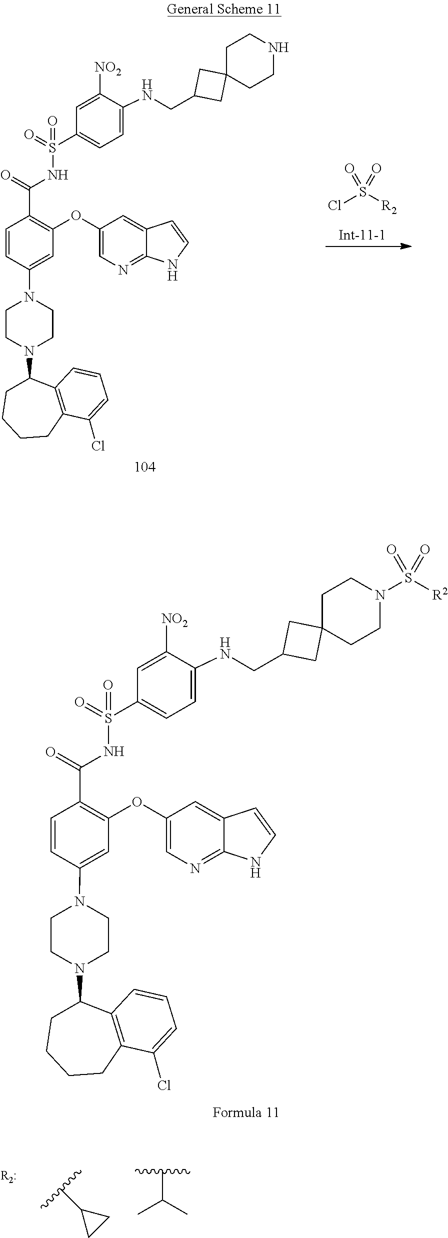

### General Step: Synthesis of Formula 11

To a solution of Compound 104 (1 mmol, 1.0 eq), DIEA (2 mmol, 2 eq) in NMP (20 mL) was added Int-11-1 (1.2 mmol, 1.2 eq). The reaction mixture was stirred at RT for 16 h. TLC showed reaction was completed. DCM was added and the organic phase was washed with brine (3×50 mL), dried over sodium sulfate and concentrated under reduced pressure. The residue was purified by flash chromatography (ME/DCM=0-10%) to give Formula 11.

### Example 40: Synthesis of Compound 127

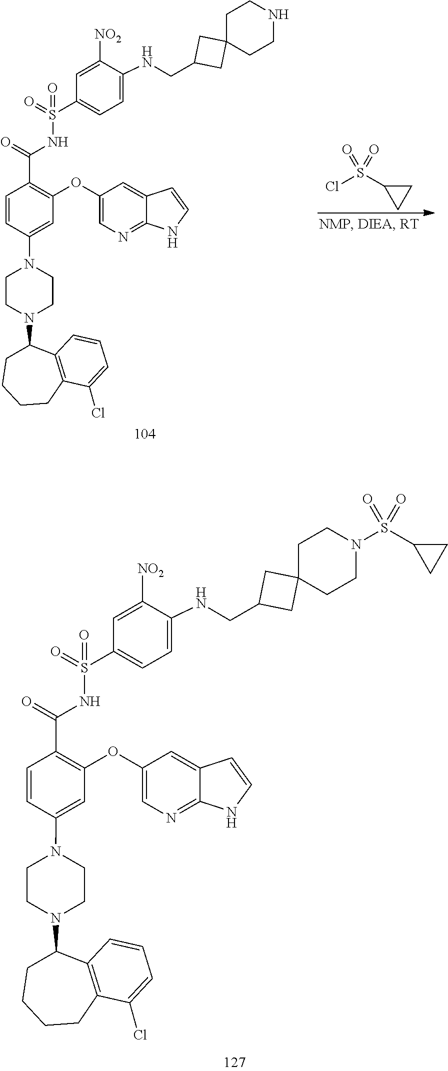 [SMILES: O=C(NS(=O)(=O)c1ccc(NCC2CC3(CCNCC3)C2)c([N+](=O)[O-])c1)c1ccc(N2CCN([C@@H]3CCCCc4c(Cl)cccc43)CC2)cc1Oc1cnc2[nH]ccc2c1 ; O=S(=O)(Cl)C1CC1 ; O=C(NS(=O)(=O)c1ccc(NCC2CC3(CCN(S(=O)(=O)C4CC4)CC3)C2)c([N+](=O)[O-])c1)c1ccc(N2CCN([C@@H]3CCCCc4c(Cl)cccc43)CC2)cc1Oc1cnc2[nH]ccc2c1]

To a solution of Compound 104 (80 mg, 0.09 mmol, 1.0 eq), DIEA (23 mg, 0.18 mmol, 2 eq) in NMP (5 mL) was added compound cyclopropanesulfonyl chloride (15 mg, 0.11 mmol, 1.2 eq). The reaction mixture was stirred at RT for 16 h. TLC showed reaction was completed. DCM was added and the organic phase was washed with brine (3×50 mL), dried over sodium sulfate and concentrated under reduced pressure. The residue was purified by flash chromatography (ME/DCM=0˜10%) to give Compound 127 (52 mg, 60% yield). MS(ESI): m/z 957.2 (M+H⁺).

Compound 128 was prepared according to the above described methods using different starting materials.

| Compound | | MS(ESI): m/z |
|---|---|---|
| ID | IUPAC Name | (M + H⁺) |
| 127 | (R)-2-((1H-pyrrolo[2,3-b]pyridin-5-yl)oxy)-4-(4-(1- | 957.2 |
| | chloro-6,7,8,9-tetrahydro-5H-benzo[7]annulen-5- | |
| | y1)piperazin-1-yl)-N-((4-(((7-(cyclopropylsulfonyl)- | |
| | 7-azaspiro[3.5]nonan-2-yl)methyl)amino)-3- | |
| | nitrophenyl)sulfonyl)benzamide | |
| 128 | (R)-2-((1H-pyrrolo[2,3-b]pyridin-5-yl)oxy)-4-(4-(1- | 959.0 |
| | chloro-6,7,8,9-tetrahydro-5H-benzo[7]annulen-5- | |
| | yl)piperazin-1-yl)-N-((4-(((7-(isopropylsulfony1)-7- | |
| | azaspiro[3.5]nonan-2-yl)methyl)amino)-3- | |
| | nitrophenyl)sulfony1)benzamide | |

### Example 41: Synthesis of Compound 132, 133, 134, 135 and 136

### Step 1: Synthesis of Int-11-2

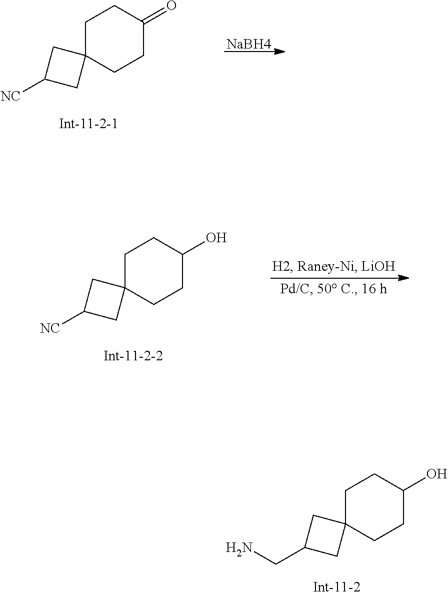 [SMILES: N#CC1CC2(CCC(=O)CC2)C1 ; "NaBH4" ; N#CC1CC2(CCC(O)CC2)C1 ; NCC1CC2(CCC(O)CC2)C1]

### Step 1-1: Synthesis of Int-11-2-2

A solution of Int-11-2-1 (100 mg, 0.61 mmol, 1 eq) in methanol (20 ml) was added NaBH₄ (44 mg, 1.22 mmol, 2 eq) at 0° C. Then the reaction mixture was stirred at rt for 3 h. LCMS showed the reaction was completed. NH₄Cl solution was added and stirred for 30 min. Then extracted with EA (20 mL×3), the organic phase was dried over Na₂SO₄, filtered and concentrated. The residue was purified by flash chromatography (ME/DCM=0˜10%) to give Compound Int-11-2-2 (92 mg, 92% yield).

### Step 1-2: Synthesis of Int-11-2

To a solution of Int-11-2-2 (92 mg, 0.56 mmol, 1.0 eq) in dioxane (20 mL) was added Raney-Ni (50 mg), Pd/C (20 mg), LiOH (25 mg, 1.2 mmol, 2 eq) and water (4 mL). The reaction mixture was stirred at 50° C. under H₂ atmosphere overnight. LCMS monitored and the starting material was consumed completely. Filtered and concentrated under vacuum to give Compound Int-11-2 (70 mg, yield 74%). MS(ESI): m/z 170.0 (M+H⁺).

### Step 2: Synthesis of Int-11-3

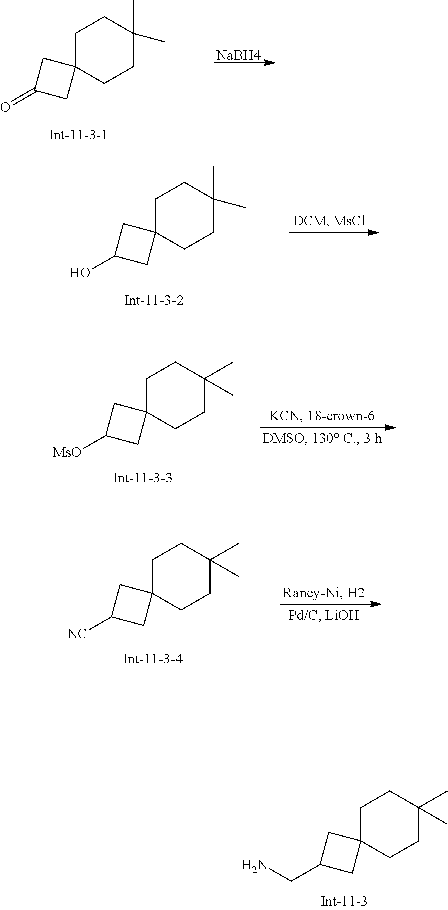 [SMILES: CC1(C)CCC2(CC1)CC(=O)C2 ; "NaBH4" ; CC1(C)CCC2(CC1)CC(O)C2 ; CC1(C)CCC2(CC1)CC(OS(C)(=O)=O)C2 ; CC1(C)CCC2(CC1)CC(C#N)C2 ; CC1(C)CCC2(CC1)CC(CN)C2]

### Step 2-1: Synthesis of Int-11-3-2

A solution of Int-11-3-1 (200 mg, 1.21 mmol, 1 eq) in methanol (30 ml) was added NaBH₄ (87 mg, 2.41 mmol, 2 eq) at 0° C. Then the reaction mixture was stirred at rt for 3 h. LCMS showed the reaction was completed. NH₄Cl solution was added and stirred for 30 min. Then extracted with EA (20 mL×3), the organic phase was dried over Na₂SO₄, filtered and concentrated. The residue was purified by flash chromatography (PE/EA=0˜50%) to give Compound Int-11-3-2 (190 mg, 95% yield).

### Step 2-2: Synthesis of Int-11-3-3

To a solution of Compound Int-11-3-2 (190 mg, 1.13 mmol, 1.0 eq), TEA (342 mg, 3.39 mmol, 3 eq) in DCM (25 mL) was added compound MsCl (260 mg, 2.26 mmol, 2 eq). The reaction mixture was stirred at RT for 16 h. TLC showed reaction was completed. DCM was added and the organic phase was washed with brine (3×50 mL), dried over sodium sulfate and concentrated under reduced pressure. The residue was purified by flash chromatography (PE/EA=0˜60%) to give Compound Int-11-3-3 (190 mg, 68% yield). MS(ESI): m/z 246.3 (M+H⁺).

### Step 2-3: Synthesis of Int-11-3-4

To a solution of Compound Int-11-3-3 (190 mg, 0.77 mmol, 1.0 eq), 18-crown-6 (284 mg, 1.15 mmol, 1.5 eq) in DMSO (15 mL) was added compound KCN (75 mg, 1.15 mmol, 1.5 eq). The reaction mixture was stirred at 130° C. for 3 h. LCMS showed reaction was completed. EA was added and the organic phase was washed with brine (3×50 mL), dried over sodium sulfate and concentrated under reduced pressure. The residue was purified by flash chromatography (PE/EA=0˜60%) to give Compound Int-11-3-4 (80 mg, 59% yield). MS(ESI): m/z 177.1 (M+H⁺).

### Step 2-4: Synthesis of Int-11-3

To a solution of Int-11-3-4 (80 mg, 0.45 mmol, 1.0 eq) in dioxane (20 mL) was added Raney-Ni (50 mg), Pd/C (20 mg), LiOH (19 mg, 0.90 mmol, 2 eq) and water (4 mL). The reaction mixture was stirred at 50° C. under H₂ atmosphere overnight. LCMS monitored and the starting material was consumed completely. Filtered and concentrated under vacuum to give Compound Int-11-3 (60 mg, yield 75%). MS(ESI): m/z 182.1 (M+H⁺).

### Step 3: Synthesis of Compound 132, 133, 134, 135, 136

Final synthesis of Compound 132, 133, 134, 135, 136 are similar to the last steps of General Scheme 8.

| Compound | | MS(ESI): m/z |
|---|---|---|
| ID | IUPAC Name | (M + H⁺) |
| 132 | (R)-2-((1H-pyrrolo[2,3-b]pyridin-5-yl)oxy)-4-(4-(1- | 852.3 |
| | chloro-6,7,8,9-tetrahydro-5H-benzo[7]annulen-5- | |
| | yl)piperazin-1-yl)-N-((3-nitro-4-((spiro[3.5]nonan-2- | |
| | ylmethyl)amino)phenyl)sulfonyl)benzamide | |
| 133 | (R)-2-((1H-pyrrolo[2,3-b]pyridin-5-yl)oxy)-N-((4- | 910.3 |
| | (((8,11-dioxadispiro[3.2.47.24]tridecan-2- | |
| | yl)methyl)amino)-3-nitrophenyl)sulfony1)-4-(4-(1- | |
| | chloro-6,7,8,9-tetrahydro-5H-benzo[7]annulen-5- | |
| | yl)piperazin-1-yl)benzamide | |
| 134 | (R)-2-((1H-pyrrolo[2,3-b]pyridin-5-yl)oxy)-4-(4-(1- | 868.3 |
| | chloro-6,7,8,9-tetrahydro-5H-benzo[7]annulen-5- | |
| | yl)piperazin-1-yl)-N-((4-(((7-hydroxyspiro[3.5]nonan-2- | |
| | yl)methyl)amino)-3-nitrophenyl)sulfonyl)benzamide | |
| 135 | (R)-2-((1H-pyrrolo[2,3-b]pyridin-5-yl)oxy)-4-(4-(1- | 888.0 |
| | chloro-6,7,8,9-tetrahydro-5H-benzo[7]annulen-5- | |
| | yl)piperazin-1-yl)-N-((4-(((7,7-difluorospiro[3.5]nonan- | |
| | 2-y1)methyl)amino)-3-nitrophenyl)sulfonyl)benzamide | |
| 136 | (R)-2-((1H-pyrrolo[2,3-b]pyridin-5-yl)oxy)-4-(4-(1- | 880.0 |
| | chloro-6,7,8,9-tetrahydro-5H-benzo[7]annulen-5- | |
| | yl)piperazin-1-y1)-N-((4-(((7,7- | |
| | dimethylspiro[3.5]nonan-2-yl)methyl)amino)-3- | |
| | nitrophenyl)sulfonyl)benzamide | |

The H-NMR data of exemplary compounds are listed below.

### Compound 1

¹H NMR (400 MHz, DMSO): δ 11.57 (s, 1H), 8.53 (s, 1H), 8.28 (d, J=7.6, 1H), 8.08 (d, J=7.6, 1H), 7.94 (d, J=2, 1H), 7.58-7.56 (m, 3H), 7.31-7.34 (m, 2H), 7.14-7.04 (m, 3H), 6.72-6.69 (m, 1H), 6.31 (s, 2H), 3.87-3.83 (m, 1H), 3.28-3.09 (m, 4H). 2.71-2.49 (m, 6H), 1.92-1.87 (m, 2H), 1.61-1.58 (m, 2H).

### Compound 1A

¹H NMR (400 MHz, DMSO): δ 11.57 (s, 1H), 8.53 (s, 1H), 8.28 (d, J=7.6, 1H), 8.08 (d, J=7.6, 1H), 7.94 (d, J=2, 1H), 7.58-7.56 (m, 3H), 7.31-7.34 (m, 2H), 7.14-7.04 (m, 3H), 6.72-6.69 (m, 1H), 6.31 (s, 2H), 3.87-3.83 (m, 1H), 3.28-3.09 (m, 4H). 2.71-2.49 (m, 6H), 1.92-1.87 (m, 2H), 1.61-1.58 (m, 2H).

### Compound 1B

¹H NMR (400 MHz, DMSO): δ 11.57 (s, 1H), 8.53 (s, 1H), 8.28 (d, J=7.6, 1H), 8.08 (d, J=7.6, 1H), 7.94 (d, J=2, 1H), 7.58-7.56 (m, 3H), 7.31-7.34 (m, 2H), 7.14-7.04 (m, 3H), 6.72-6.69 (m, 1H), 6.31 (s, 2H), 3.87-3.83 (m, 1H), 3.28-3.09 (m, 4H). 2.71-2.49 (m, 6H), 1.92-1.87 (m, 2H), 1.61-1.58 (m, 2H).

### Compound 2

¹H NMR (400 MHz, DMSO): δ 11.65 (s, 1H), 8.52 (br, 1H) 8.02 (br, 2H), 7.76-7.74 (m, 1H), 7.57-7.48 (m, 4H), 7.10-7.03 (m, 4H), 6.73-6.70 (m, 1H), 6.36 (s, 1H), 6.26 (s, 1H), 3.82-3.86 (s, 3H), 3.38-3.12 (m, 8H), 2.75-2.43 (m, 7H), 1.82-1.78 (m, 3H). 1.56-1.43 (m, 4H), 1.26-1.21 (m, 2H),

### Compound 3

¹H NMR (400 MHz, DMSO): δ 11.59 (s, 1H), 8.52 (s, 1H), 8.27 (d, J=8 Hz, 1H), (m, 3H), 8.07 (d, J=6.8 Hz, 1H), 7.61-7.21 (m, 9H), 6.71 (dd, J=1.2, 8.8 Hz, 1H), 6.32 (s, 2H), 4.55-4.52 (m, 1H), 3.35-3.14 (m, 4H), 2.95-2.65 (m, 6H), 2.14-2.08 (m, 2H).

### Compound 3A

¹H NMR (400 MHz, DMSO): δ 11.59 (s, 1H), 8.52 (s, 1H), 8.27 (d, J=8 Hz, 1H), (m, 3H), 8.07 (d, J=6.8 Hz, 1H), 7.61-7.21 (m, 9H), 6.71 (dd, J=1.2, 8.8 Hz, 1H), 6.32 (s, 2H), 4.55-4.52 (m, 1H), 3.35-3.14 (m, 4H), 2.95-2.65 (m, 6H), 2.14-2.08 (m, 2H).

### Compound 3B

¹H NMR (400 MHz, DMSO): δ 11.59 (s, 1H), 8.52 (s, 1H), 8.27 (d, J=8 Hz, 1H), (m, 3H), 8.07 (d, J=6.8 Hz, 1H), 7.61-7.21 (m, 9H), 6.71 (dd, J=1.2, 8.8 Hz, 1H), 6.32 (s, 2H), 4.55-4.52 (m, 1H), 3.35-3.14 (m, 4H), 2.95-2.65 (m, 6H), 2.14-2.08 (m, 2H).

### Compound 4

¹H NMR (400 MHz, DMSO): δ 11.57 (s, 1H), 8.42 (br, 2H), 7.95 (s, 1H), 7.64-7.43 (m, 2H), 7.32 (s, 1H), 7.28-7.15 (m, 5H), 6.87 (br, 1H), 6.31 (s, 1H), 6.27 (s, 1H), 4.31-4.28 (m, 1H), 3.85-3.82 (m, 2H), 3.28-3.23 (m, 4H). 3.08 (br, 4H), 2.87-2.71 (m, 2H), 2.42-2.39 (m, 2H), 1.99-1.87 9m, 3H), 1.62-1.64 (m, 2H), 1.32-1.26 (m, 4H)

### Compound 5

¹H NMR (400 MHz, DMSO): δ 11.61 (s, 1H), 8.57 (s, 1H), 8.33 (d, J=7.6 Hz, 1H), 8.14 (d, J=7.6 Hz, 1H), 7.96 (d, J=2, 1H), 7.68-7.38 (m, 4H), 7.08-7.06 (m, 4H), 6.69 (d, J=8.8, 1H), 6.32-6.28 (m, 2H), 3.34-3.41 (m, 3H), 3.17-3.12 (m, 3H), 2.49-2.45 (m, 2H), 2.10-1.83 (m, 5H), 1.60-1.22 (m, 4H).

### Compound 5A

¹H NMR (400 MHz, DMSO): δ 11.61 (s, 1H), 8.57 (s, 1H), 8.33 (d, J=7.6 Hz, 1H), 8.14 (d, J=7.6 Hz, 1H), 7.96 (d, J=2, 1H), 7.68-7.38 (m, 4H), 7.08-7.06 (m, 4H), 6.69 (d, J=8.8, 1H), 6.32-6.28 (m, 2H), 3.34-3.41 (m, 3H), 3.17-3.12 (m, 3H), 2.49-2.45 (m, 2H), 2.10-1.83 (m, 5H), 1.60-1.22 (m, 4H).

### Compound 5B

¹H NMR (400 MHz, DMSO): δ 11.61 (s, 1H), 8.57 (s, 1H), 8.33 (d, J=7.6 Hz, 1H), 8.14 (d, J=7.6 Hz, 1H), 7.96 (d, J=2, 1H), 7.68-7.38 (m, 4H), 7.08-7.06 (m, 4H), 6.69 (d, J=8.8, 1H), 6.32-6.28 (m, 2H), 3.34-3.41 (m, 3H), 3.17-3.12 (m, 3H), 2.49-2.45 (m, 2H), 2.10-1.83 (m, 5H), 1.60-1.22 (m, 4H).

### Compound 6

¹H NMR (400 MHz, DMSO): δ 11.66 (s, 1H), 8.49 (s, 1H), 7.99 (s, 1H), 7.76-6.25 (m, 12H), 5.86-5.84 (m, 1H), 5.31-5.34 (m, 1H), 3.87-3.83 (m, 2H), 3.51-3.05 (m, 8H), 2.96-2.89 (m, 2H), 2.76-2.56 (m, 4H), 2.16-1.83 (m, 6H), 1.69-1.47 (m, 6H).

### Compound 6A

¹H NMR (400 MHz, DMSO): δ 11.66 (s, 1H), 8.49 (s, 1H), 7.99 (s, 1H), 7.76-6.25 (m, 12H), 5.86-5.84 (m, 1H), 5.31-5.34 (m, 1H), 3.87-3.83 (m, 2H), 3.51-3.05 (m, 8H), 2.96-2.89 (m, 2H), 2.76-2.56 (m, 4H), 2.16-1.83 (m, 6H), 1.69-1.47 (m, 6H).

### Compound 6B

¹H NMR (400 MHz, DMSO): δ 11.66 (s, 1H), 8.49 (s, 1H), 7.99 (s, 1H), 7.76-6.25 (m, 12H), 5.86-5.84 (m, 1H), 5.31-5.34 (m, 1H), 3.87-3.83 (m, 2H), 3.51-3.05 (m, 8H), 2.96-2.89 (m, 2H), 2.76-2.56 (m, 4H), 2.16-1.83 (m, 6H), 1.69-1.47 (m, 6H).

### Compound 7

¹H NMR (400 MHz, DMSO): δ 11.51 (s, 1H), 8.43 (d, J=4.8 Hz, 1H), 8.11-8.20 (m, 3H), 7.85-7.93 (m, 2H), 7.55-7.65 (m, 3H), 7.12-7.46 (m, 5H), 6.95 (d, J=6.0 Hz, 2H), 6.71 (d, J=9.2 Hz, 1H), 6.20-6.36 (m, 2H), 5.57 (s, 1H), 3.51 (t, J=8.0 Hz, 2H), 2.66-3.09 (m, 7H), 2.30-2.34 (m, 1H), 2.18-2.22 (m, 1H), 1.97-2.02 (m, 1H), 1.84-1.89 (m, 1H)

### Compound 8

¹H NMR (400 MHz, DMSO): 11.27 (s, 1H), 8.63 (s, 1H), 8.23 (br, 1H)), 8.31 (br, 1H), 7.93 (s, 1H), 7.52-7.51 (m, 2H), 7.46-7.43 (m, 2H), 7.30-7.21 (m, 7H), 6.91-7.19 (m, 2H), 6.55-6.56 (m, 1H), 6. (br, 1H). 6.12 (s, 1H), 3.95-4.01 (m, 1H), 2.82-2.51 (m, 6H), 1.29-2.17 (m, 2H), 1.98 (br, 2H), 1.58-1.53 (m, 2H).

### Compound 9

¹H NMR (400 MHz, DMSO) δ 11.53 (s, 1H), 8.50 (s, 1H), 8.22 (d, J=7.6 Hz, 1H), 8.01 (d, J=6.4 1H), 7.91 (d, J=2.0 Hz, 1H), 7.65-7.21 (m, 5H), 7.09 (t, J=7.5 Hz, 1H), 6.85-6.82 (m, 1H), 6.70-6.31 m, 2H), 6.31-6.29 (m, 2H), 4.28-4.26 (m, 1H), 4.10-4.05 (m, 1H), 3.87-3.86 (m, 1H), 3.12 (br, 4H), 2.59-2.58 (m, 4H), 1.97-1.86 (m, 2H).

### Compound 10

¹H NMR (400 MHz, DMSO) δ 11.53 (s, 1H), 8.53 (s, 1H), 8.27 (d, J=4.8 Hz, 1H), 8.07 (br, 1H), 7.93 (s, 1H), 7.60-7.43 (m, 4H), 7.32 (br, 2H), 7.23-7.05 (m, 1H), 6.70-6.67 m, 1H), 6.30 (br, 2H), 4.71-4.52 (m, 2H), 4.18-4.16 (m, 1H), 3.73-3.70 (m, 2H), 3.14-3.03 (m, 4H), 2.58-2.46 (m, 4H).

### Compound 11

¹H NMR (400 MHz, DMSO): 11.61 (s, 1H), 8.56 (s, 1H), 8.34 (d, J=7.2, 1H)), (d, J=6.8, 1H), 7.95 (s, 1H), 7.69-7.32 (m, 8H), 7.21-7.01 (m, 3H), 6.59 (d, J=7.6, 1H), 6.33 (s, 1H), 6.15 (s, 1H), 4.05 (br, 1H). 2.86-2.58 (m, 6H), 2.47-1.50 (m, 4H), 1.92-1.60 (m, 4H).

### Compound 12

¹H NMR (400 MHz, DMSO) δ 11.65 (s, 1H), 8.53 (d, J=10.4 Hz, 2H), 8.02 (s, 1H), 7.74 (d, J-8.4, 1H), 7.52-7.46 (m, 4H), 7.25-7.18 (m, 2H), 7.05 (br, 2H), 6.69 (dd, J=2.0, 8.8, 1H), 6.36 (s, 1H), 6.24 (s, 1H), 4.67 (d, J=14.9 Hz, 1H), 4.53 (d, J=15.1 Hz, 1H), 4.12-4.11 (m, 1H), 3.86-3.83 (m, 2H), 3.71-3.68 (m, 2H), 3.30-3.02 (m, 9H), 2.69-2.50 (m, 3H), 1.88-1.86 (m, 1H), 1.61 (d, J=13.7 Hz, 2H), 1.25-1.24 (m, 2H).

### Compound 13

¹H NMR (400 MHz, DMSO): 11.71 (s, 1H), 8.55 (s, 1H), 8.33 (d, J=2.0, 1H)), 8.31 (br, 1H), 8.12 (s, 1H), 7.93-6.93 (m, 12H), 6.94 (d, J=7.2, 1H), 6.31 (s, 1H), 6.13 (s, 1H), 3.99 (s, 1H), 2.84-2.51 (m, 6H). 2.51-1.58 (m, 8H).

### Compound 14

¹H NMR (400 MHz, DMSO): 11.75 (s, 1H), 8.56 (s, 1H), 8.35-8.32 (m, 1H)), 8.32 (br, 1H), 8.13 (s, 1H), 7.95-6.86 (m, 12H), 6.85 (d, J=7.2, 1H), 6.38 (s, 1H), 6.15 (s, 1H), 3.99 (s, 1H), 2.84-2.51 (m, 6H). 2.51-1.48 (m, 4H), 1.42-1.28 (m, 4H).

### Compound 15

¹H NMR (400 MHz, DMSO): 11.59 (s, 1H), 8.55 (s, 1H), 8.32 (d, J=7.2, 1H), 8.13 (d, J=7.2, 1H), 7.94 (s, 1H), 7.65 (br, 1H), 7.51-7.45 (m, 4H), 7.20-7.01 (m, 5H), 6.59 (d, J=9.2, 1H), 6.17 (s, 1H), 4.16 (s, 1H), 2.85-2.63 (m, 6H). 2.31-2.19 (m, 4H), 1.79-1.23 (m, 4H).

### Compound 16

¹H NMR (400 MHz, DMSO): 11.67 (s, 1H), 11.46 (s, 1H), 8.58-8.53 (m, 2H), 8.00 (s, 1H), 7.77-7.76 (m, 1H), 7.48-6.92 (m, 12H), 6.57 (d, J=2.2, 1H), 6.36 (s, 1H), 6.09 (s, 1H), 3.98 (br, 1H), 3.86-3.83 (m, 1H), 3.38-3.23 (m, 4H), 2.84-2.56 (m, 6H). 2.21-1.97 (m, 4H), 1.94-1.71 (m, 3H), 1.67-1.54 (m, 4H), 1.30-1.18 (m, 2H).

### Compound 17

¹H NMR (400 MHz, DMSO): 11.52 (s, 1H), 8.39-8.35 (m, 2H), 8.17 (s, 1H), 7.93 (s, 1H), 7.60 (d, J=8.8 Hz, 2H), 7.39-7.41 (m, 1H), 7.27 (s, 1H), 7.09 (t, J=7.2, 1H), 6.83-6.80 m, 2H), 6.72-6.66 (m, 2H), 6.30 (s, 2H), 4.29-4.26 (m, 1H), 4.40-4.07 (m, 1H), 3.85-3.83 (m, 1H), 3.54-3.09 (m, 11H), 2.60-2.50 (m, 2H), 1.96-1.62 (m, 3H), 1.62-1.59 (m, 2H), 1.59-1.25 (m, 3H).

### Compound 18

¹H NMR (400 MHz, DMSO) δ 11.67 (s, 1H), 11.51 (s, 1H), 8.61 (t, J=6.0 Hz, 1H), 8.56 (d, J=2.0 Hz, 1H), 8.04 (d, J=2.4 Hz, 1H), 7.80 (dd, J=9.6, 1.6 Hz, 1H), 7.55-7.47 (m, 3H), 7.45 (d, J=8.0 Hz, 1H), 7.12 (t, J=8.2 Hz, 2H), 6.99 (t, J=7.6 Hz, 1H), 6.71 (dd, J=9.2, 2.0 Hz, 1H), 6.38 (dd, J=3.2, 2.0 Hz, 1H), 6.23 (d, J=2.0 Hz, 1H), 3.85 (dd, J=11.2, 2.8 Hz, 2H), 3.49 (t, J=12.4 Hz, 1H), 3.30-3.19 (m, 5H), 3.19-3.08 (m, 4H), 3.07-2.98 (m, 1H), 2.48-2.42 (m, 1H), 2.16-2.03 (m, 3H), 1.98-1.77 (m, 3H), 1.66-1.45 (m, 4H), 1.33-1.12 (m, 4H).

### Compound 18A

¹H NMR (400 MHz, DMSO) δ 11.67 (s, 1H), 11.51 (s, 1H), 8.61 (t, J=6.0 Hz, 1H), 8.56 (d, J=2.0 Hz, 1H), 8.04 (d, J=2.4 Hz, 1H), 7.80 (dd, J=9.6, 1.6 Hz, 1H), 7.55-7.47 (m, 3H), 7.45 (d, J=8.0 Hz, 1H), 7.12 (t, J=8.2 Hz, 2H), 6.99 (t, J=7.6 Hz, 1H), 6.71 (dd, J=9.2, 2.0 Hz, 1H), 6.38 (dd, J=3.2, 2.0 Hz, 1H), 6.23 (d, J=2.0 Hz, 1H), 3.85 (dd, J=11.2, 2.8 Hz, 2H), 3.49 (t, J=12.4 Hz, 1H), 3.30-3.19 (m, 5H), 3.19-3.08 (m, 4H), 3.07-2.98 (m, 1H), 2.48-2.42 (m, 1H), 2.16-2.03 (m, 3H), 1.98-1.77 (m, 3H), 1.66-1.45 (m, 4H), 1.33-1.12 (m, 4H).

### Compound 18B

¹H NMR (400 MHz, DMSO) δ 11.67 (s, 1H), 11.51 (s, 1H), 8.61 (t, J=6.0 Hz, 1H), 8.56 (d, J=2.0 Hz, 1H), 8.04 (d, J=2.4 Hz, 1H), 7.80 (dd, J=9.6, 1.6 Hz, 1H), 7.55-7.47 (m, 3H), 7.45 (d, J=8.0 Hz, 1H), 7.12 (t, J=8.2 Hz, 2H), 6.99 (t, J=7.6 Hz, 1H), 6.71 (dd, J=9.2, 2.0 Hz, 1H), 6.38 (dd, J=3.2, 2.0 Hz, 1H), 6.23 (d, J=2.0 Hz, 1H), 3.85 (dd, J=11.2, 2.8 Hz, 2H), 3.49 (t, J=12.4 Hz, 1H), 3.30-3.19 (m, 5H), 3.19-3.08 (m, 4H), 3.07-2.98 (m, 1H), 2.48-2.42 (m, 1H), 2.16-2.03 (m, 3H), 1.98-1.77 (m, 3H), 1.66-1.45 (m, 4H), 1.33-1.12 (m, 4H).

### Compound 19

¹H NMR (400 MHz, DMSO): 11.67 (s, 1H), 11.51 (br, 1H), 8.60-8.55 (m, 2H), 8.02 (s, 1H), 7.77 (d, J=7.6, 1H), 7.59-7.02 (m, 12H), 6.72-6.37 (m, 1H), 6.37 (s, 1H), 6.27 (s, 1H), 3.86-3.82 (m, 2H), 3.31-3.13 (m, 11H). 2.15-1.85 (m, 11H), 1.30-1.26 (m, 4H).

### Compound 20

¹H NMR (400 MHz, DMSO): 11.63 (s, 1H), 8.52-8.50 (m, 2H), 8.02 (s, 1H), 7.53 (d, J=8.8, 1H), 7.52-7.44 (m, 3H), 6.99-6.91 (m, 4H), 6.69 (d, J=8.00, 1H), 6.35 (s, 1H), 6.24 (s, 1H), 3.86-3.82 (m, 2H), 3.29-2.70 (m, 11H). 2.70-2.67 (m, 1H), 2.22 (s, 3H), 2.12-2.09 (m, 3H), 1.92-1.85 (m, 3H), 1.62-1.26 (m, 11H).

### Compound 21

¹H NMR (400 MHz, DMSO) δ 11.64 (s, 1H), 8.53 (s, 2H), 8.02 (s, 1H), 7.76 (d, J=8.4 Hz, 1H), 7.53 (dd, J=26.0, 17.2 Hz, 3H), 7.28 (dd, J=11.0, 3.1 Hz, 2H), 7.03 (d, J=8.0 Hz, 2H), 6.70 (d, J=8.8 Hz, 1H), 6.41-6.38 (m, 2H), 3.85 (d, J=7.9 Hz, 2H), 3.33-3.22 (m, 5H), 3.20-2.99 (m, 4H), 2.33 (s, 3H), 2.08 (s, 2H), 2.00-1.73 (m, 3H), 1.67-1.38 (m, 4H), 1.33-1.10 (m, 5H).

### Compound 22

¹H NMR (400 MHz, DMSO) δ 11.57 (s, 1H), 8.46 (s, 2H), 7.97 (s, 1H), 7.72-7.51 (m, 4H), 7.48-7.26 (m, 6H), 7.15 (d, J=7.9 Hz, 1H), 6.93 (s, 1H), 6.68 (d, J=8.9 Hz, 1H), 6.32-6.26 (m, 2H), 3.86-3.832 (m, 3H), 3.29-3.23 (m, 7H), 3.11 (br, 4H), 2.14 (br, 3H), 2.07-1.79 (m, 4H), 1.57-62-1.59 (m, 3H), 1.29-1.23 (m, 4H).

### Compound 23

¹H NMR (400 MHz, DMSO) δ 11.63 (s, 1H), 8.51 (s, 2H), 8.01 (s, 1H), 7.73 (br, 1H), 7.53-7.46 (m, 3H), 7.28 (br, 2H), 7.06 (d, J=6.2 Hz, 2H), 6.70 (s, 1H), 6.35-6.24 (m, 2H), 3.84 (br, 2H), 3.15-3.10 (m, 9H), 2.47-2.39 (m, 2H), 1.97-1.89 (m, 7H), 1.53-1.26 (m, 8H).

### Compound 24

¹H NMR (400 MHz, DMSO) δ 12.11 (s, 1H), 9.10 (s, 1H), 8.74-8.51 (m, 2H), 8.14 (s, 1H), 7.94 (dd, J=9.2, 2.2 Hz, 1H), 7.77 (d, J=7.5 Hz, 2H), 7.26 (d, J=9.4 Hz, 3H), 6.98 (s, 2H), 4.15-4.11 (m, 9H), 3.50 (s, 2H), 3.45-3.27 (m, 4H), 3.09 (d, J=5.4 Hz, 4H), 3.02-2.77 (m, 2H), 2.69-2.56 (m, 2H), 2.10-2.04 (m, 3H), 1.95-1.75 (m, 8H), 1.65-1.61 (m, 5H).

### Compound 25

¹H NMR (400 MHz, DMSO) δ 11.66 (s, 1H), 11.42 (s, 1H), 8.55-8.46 (m, 2H), 8.03 (d, J=2.4 Hz, 1H), 7.78 (d, J=8.8 Hz, 1H), 7.59-7.39 (m, 4H), 7.25-6.98 (m, 5H), 6.77-6.56 (m, 1H), 6.45-6.13 (m, 2H), 5.29 (d, J=4.4 Hz, 1H), 4.72 (dd, J=9.6, 3.6 Hz, 1H), 3.85-3.83 (m, 2H), 3.29-3.23 (m, 7H), 2.50-2.49 (m, 2H), 2.07-2.06 (m, 2H), 2.07-1.23 (m, 13H)

### Compound 26

¹H NMR (400 MHz, DMSO): 11.66 (s, 1H), 11.61 (s, 1H), 8.58-8.54 (m, 2H), 8.02 (d, J=2.4, 1H), 7.77 (d, J=9.2, 1H), 7.53-7.48 (m, 3H), 7.17-7.07 (m, 4H), 6.70-6.68 (m, 1H), 6.37 (s, 1H), 6.23 (s, 1H), 3.86-3.83 (m, 2H), 3.29-3.11 (m, 10H). 2.40-1.81 (m, 8H), 1.63-1.24 (m, 8H).

### Compound 27A

¹H NMR (400 MHz, DMSO): δ 11.61 (s, 1H), 8.48 (s, 2H), 8.15 (s, 1H), 7.99 (s, 1H), 7.50-7.71 (m, 2H), 7.40-7.50 (m, 2H), 7.27 (d, J=6.0 Hz, 1H), 7.00-7.15 (m, 3H), 6.69 (d, J=7.2 Hz, 1H), 6.34 (s, 1H), 6.26 (s, 1H), 3.40-3.90 (m, 2H), 3.00-3.40 (m, 11H), 1.30-2.20 (m, 15H)

### Compound 27B

¹H NMR (400 MHz, DMSO): δ 11.61 (s, 1H), 8.48 (s, 2H), 8.15 (s, 1H), 7.99 (s, 1H), 7.50-7.71 (m, 2H), 7.40-7.50 (m, 2H), 7.27 (d, J=6.0 Hz, 1H), 7.00-7.15 (m, 3H), 6.69 (d, J=7.2 Hz, 1H), 6.34 (s, 1H), 6.26 (s, 1H), 3.40-3.90 (m, 2H), 3.00-3.40 (m, 11H), 1.30-2.20 (m, 15H)

### Compound 28

¹H NMR (400 MHz, CDCl₃) δ 10.08 (s, 1H), 9.63 (s, 1H), 8.82 (br, 1H), 8.22-8.17 (m, 1H), 8.12-7.99 (m, 2H), 7.91 (d, J=9.1 Hz, 1H), 7.67-7.65 (m, 2H), 7.54-7.33 (m, 2H), 7.08-7.06 (m, 1H), 6.94-6.79 (m, 2H), 6.52-6.49 (m, 2H), 4.23-3.99 (m, 2H), 3.49-3.15 (m, 10H), 2.23-1.97 (m, 8H), 1.82-1.78 (m, 4H).

### Compound 29

¹H NMR (400 MHz, DMSO): δ 11.6 (s, 1H), 8.40-8.50 (m, 2H), 7.94 (d, J=2.0 Hz, 1H), 7.50-7.70 (m, 2H), 7.73 (t, J=2.8 Hz 2H), 7.25-7.35 (m, 2H), 7.00-7.18 (m, 3H), 6.66 (dd, J=2.4, 4.8 Hz, 1H), 6.20-6.35 (m, 2H), 3.0-3.4 (m, 6H), 2.5-2.8 (m, 4H), 1.8-2.3 (m, 9H), 1.3-1.6 (m, 12H).

### Compound 30

¹H NMR (400 MHz, DMSO) δ 11.64 (s, 1H), 8.51-8.49 (m, 2H), 8.07-7.92 (m, 3H), 7.74 (d, J=8.6 Hz, 1H), 7.58-7.32 (m, 4H), 7.04 (br, 1H), 6.70 (d, J=6.9 Hz, 1H), 6.35 (s, 1H), 6.32 (s, 1H) 3.85 (d, J=8.1 Hz, 2H), 3.57-3.39 (m, 2H), 3.37-3.20 (m, 8H), 3.09 (d, J=28.3 Hz, 3H), 2.74-2.60 (m, 1H), 2.11 (dd, J=14.5, 8.7 Hz, 2H), 2.02-1.77 (m, 3H), 1.57 (m, 3H), 1.29 (m, 4H).

### Compound 31

¹H NMR (500 MHz, DMSO) δ 11.69 (s, 1H), 11.43 (s, 1H), 8.86-8.73 (m, 1H), 8.62-8.53 (m, 1H), 8.03 (d, J=2.5 Hz, 1H), 7.81 (d, J=9.0 Hz, 1H), 7.52 (s, 1H), 7.49 (d, J=4.5 Hz, 2H), 7.27 (d, J=7.0 Hz, 1H), 7.11-7.01 (m, 3H), 6.70 (d, J=9.0 Hz, 1H), 6.37 (s, 1H), 6.23 (s, 1H), 3.66-3.56 (m, 4H), 3.53-3.45 (m, 2H), 3.42-3.36 (m, 3H), 3.25-3.21 (m, 1H), 3.18-3.09 (m, 4H), 3.07-3.00 (m, 1H), 2.73-2.62 (m, 2H), 2.59-2.54 (m, 2H), 2.48-2.43 (m, 2H), 2.15-2.03 (m, 3H), 2.00-1.90 (m, 1H), 1.88-1.80 (m, 1H), 1.64-1.54 (m, 1H), 1.49 (t, J=13.0 Hz, 1H), 1.25-1.12 (m, 1H).

### Compound 31A

¹H NMR (500 MHz, DMSO) δ 11.69 (s, 1H), 11.43 (s, 1H), 8.86-8.73 (m, 1H), 8.62-8.53 (m, 1H), 8.03 (d, J=2.5 Hz, 1H), 7.81 (d, J=9.0 Hz, 1H), 7.52 (s, 1H), 7.49 (d, J=4.5 Hz, 2H), 7.27 (d, J=7.0 Hz, 1H), 7.11-7.01 (m, 3H), 6.70 (d, J=9.0 Hz, 1H), 6.37 (s, 1H), 6.23 (s, 1H), 3.66-3.56 (m, 4H), 3.53-3.45 (m, 2H), 3.42-3.36 (m, 3H), 3.25-3.21 (m, 1H), 3.18-3.09 (m, 4H), 3.07-3.00 (m, 1H), 2.73-2.62 (m, 2H), 2.59-2.54 (m, 2H), 2.48-2.43 (m, 2H), 2.15-2.03 (m, 3H), 2.00-1.90 (m, 1H), 1.88-1.80 (m, 1H), 1.64-1.54 (m, 1H), 1.49 (t, J=13.0 Hz, 1H), 1.25-1.12 (m, 1H).

### Compound 31B

¹H NMR (500 MHz, DMSO) δ 11.69 (s, 1H), 11.43 (s, 1H), 8.86-8.73 (m, 1H), 8.62-8.53 (m, 1H), 8.03 (d, J=2.5 Hz, 1H), 7.81 (d, J=9.0 Hz, 1H), 7.52 (s, 1H), 7.49 (d, J=4.5 Hz, 2H), 7.27 (d, J=7.0 Hz, 1H), 7.11-7.01 (m, 3H), 6.70 (d, J=9.0 Hz, 1H), 6.37 (s, 1H), 6.23 (s, 1H), 3.66-3.56 (m, 4H), 3.53-3.45 (m, 2H), 3.42-3.36 (m, 3H), 3.25-3.21 (m, 1H), 3.18-3.09 (m, 4H), 3.07-3.00 (m, 1H), 2.73-2.62 (m, 2H), 2.59-2.54 (m, 2H), 2.48-2.43 (m, 2H), 2.15-2.03 (m, 3H), 2.00-1.90 (m, 1H), 1.88-1.80 (m, 1H), 1.64-1.54 (m, 1H), 1.49 (t, J=13.0 Hz, 1H), 1.25-1.12 (m, 1H).

### Compound 32

¹H NMR (400 MHz, DMSO): δ 11.60 (s, 1H), 8.61 (s, 1H), 8.47 (d, J=2.0, 1H), 7.97 (d, J=2.4 Hz, 1H), 7.50-7.70 (m, 1H), 7.40-7.18 (m, 1H), 7.20-7.40 (m, 2H), 7.00-7.15 (m, 2H), 6.20-6.90 (m, 4H), 3.36-3.43 (m, 4H), 3.03-3.11 (m, 8H), 2.54-2.69 (m, 8H), 2.09-2.12 (m, 6H), 1.19-2.09 (m 6H).

### Compound 33

¹H NMR (400 MHz, DMSO) δ 11.51 (s, 1H), 8.33 (dd, J=13.5, 7.8 Hz, 4H), 7.91 (d, J=2.5 Hz, 1H), 7.58 (dd, J=18.8, 10.5 Hz, 3H), 7.47 (d, J=6.9 Hz, 1H), 7.40 (t, J=2.8 Hz, 1H), 7.31 (t, J=7.8 Hz, 1H), 7.22 (d, J=2.3 Hz, 1H), 6.76 (d, J=9.1 Hz, 1H), 6.72-6.58 (m, 1H), 6.38-6.08 (m, 2H), 3.89-3.79 (m, 3H), 3.35-3.17 (m, 8H), 3.09 (s, 6H), 2.17-2.08 (m, 3H), 1.98-1.86 (m, 4H), 1.62-1.52 (m, 4H).

### Compound 34

¹H NMR (400 MHz, DMSO) δ 11.50 (s, 1H), 9.80 (s, 1H), 8.39-8.27 (m, 2H), 7.90 (d, J=2.6 Hz, 1H), 7.60 (d, J=8.7 Hz, 1H), 7.54 (d, J=8.8 Hz, 1H), 7.38 (d, J=7.4 Hz, 2H), 7.26 (s, 1H), 7.20 (d, J=2.6 Hz, 1H), 6.96 (d, J=7.9 Hz, 1H), 6.74 (d, J=9.1 Hz, 1H), 6.66 (d, J=9.4 Hz, 1H), 6.36-6.20 (m, 2H), 3.93-3.73 (m, 2H), 3.30-3.20 (m, 6H), 3.07 (s, 5H), 2.14 (s, 3H), 1.99 (s, 4H), 1.83 (s, 2H), 1.67-1.47 (m, 3H), 1.34-1.23 (m, 5H).

### Compound 35

¹H NMR (400 MHz, DMSO) δ 11.61 (s, 2H), 8.50 (s, 2H), 7.99 (s, 1H), 7.81-7.67 (m, 1H), 7.66-7.33 (m, 8H), 7.22-7.02 (m, 5H), 6.72 (s, 1H), 6.32 (d, J=19.2 Hz, 2H), 4.09 (s, 1H), 3.84 (d, J=11.2 Hz, 4H), 3.62 (s, 1H), 3.25 (d, J=11.7 Hz, 2H), 3.11 (s, 4H), 2.68 (s, 3H), 2.34 (s, 5H), 2.25-2.21 (m, 4H), 1.88 (s, 1H), 1.61 (d, J=13.1 Hz, 3H), 1.30-1.20 (m, 4H).

### Compound 36

¹H NMR (400 MHz, DMSO) δ 11.69 (s, 1H), 11.49 (s, 1H), 8.61 (t, J=6.0 Hz, 1H), 8.56 (d, J=2.4 Hz, 1H), 8.05 (d, J=2.4 Hz, 1H), 7.80 (dd, J=9.2, 2.0 Hz, 1H), 7.51 (m, 3H), 7.24 (d, J=6.4 Hz, 1H), 7.19-7.07 (m, 2H), 6.98 (t, J=7.2 Hz, 1H), 6.90 (d, J=8.0 Hz, 1H), 6.71 (dd, J=9.2, 2.4 Hz, 1H), 6.39 (dd, J=3.2, 2.0 Hz, 1H), 6.24 (d, J=2.0 Hz, 1H), 4.20-4.11 (m, 1H), 3.85 (dd, J=11.2, 2.8 Hz, 2H), 3.78 (m, 1H), 3.34-3.22 (m, 5H), 3.20-3.05 (m, 4H), 2.60-2.53 (m, 1H), 2.33-2.21 (m, 2H), 2.12-2.02 (m, 1H), 1.99-1.84 (m, 2H), 1.82-1.71 (m, 1H), 1.67-1.57 (m, 3H), 1.32-1.19 (m, 3H).

### Compound 37

¹H NMR (400 MHz, DMSO) δ 11.64 (s, 1H), 11.52 (s, 1H), 8.51 (s, 2H), 8.01 (s, 1H), 7.79-7.69 (m, 1H), 7.53 (d, J=8.8 Hz, 1H), 7.50-7.41 (m, 3H), 7.13-6.85 (m, 5H), 6.70 (d, J=7.6 Hz, 1H), 6.36 (s, 1H), 6.25 (s, 1H), 3.85 (d, J=8.4 Hz, 2H), 3.31-3.23 (m, 5H), 3.18-3.04 (m, 6H), 2.16-2.03 (m, 3H), 1.98-1.76 (m, 3H), 1.66-1.49 (m, 4H), 1.35-1.18 (m, 4H), 0.94 (t, J=7.2 Hz, 1H).

### Compound 38

¹H NMR (400 MHz, DMSO) δ 11.68 (s, 1H), 11.52 (s, 1H), 8.62-8.53 (m, 2H), 8.04 (d, J=2.4 Hz, 1H), 7.79 (d, J=9.2 Hz, 1H), 7.57-7.46 (m, 3H), 7.28 (dd, J=7.2, 2.4 Hz, 1H), 7.11-7.05 (m, 3H), 6.71 (d, J=9.2 Hz, 1H), 6.41-6.36 (m, 1H), 6.22 (d, J=2.0 Hz, 1H), 4.29 (s, 2H), 4.19 (s, 2H), 3.27-3.20 (m, 4H), 3.17-3.01 (m, 5H), 2.14-2.02 (m, 5H), 1.98-1.80 (m, 2H), 1.70-1.63 (m, 2H), 1.62-1.42 (m, 3H), 1.42-1.33 (m, 2H), 1.27-1.14 (m, 3H), 1.01-0.90 (m, 2H).

### Compound 39

¹H NMR (400 MHz, DMSO) δ 11.68 (s, 1H), 11.50 (s, 1H), 8.85-8.78 (m, 1H), 8.56 (d, J=2.2 Hz, 1H), 8.03 (d, J=2.6 Hz, 1H), 7.82 (d, J=9.5 Hz, 1H), 7.57-7.48 (m, 3H), 7.28 (dd, J=7.1, 2.3 Hz, 1H), 7.10-7.02 (m, 3H), 6.71 (d, J=11.2 Hz, 1H), 6.38 (dd, J=3.3, 1.9 Hz, 1H), 6.23 (d, J=2.1 Hz, 1H), 3.52-3.39 (m, 8H), 3.26-3.03 (m, 8H), 2.72-2.64 (m, 2H), 2.46-2.40 (m, 2H), 2.15-2.05 (m, 3H), 2.00 (s, 3H), 1.88-1.82 (m, 1H), 1.62-1.56 (m, 1H), 1.51-1.44 (m, 1H), 1.25-1.15 (m, 3H).

### Compound 40

¹H NMR (500 MHz, DMSO) δ 11.48 (s, 1H), 9.11 (s, 1H), 8.35 (d, J=2.0 Hz, 1H), 8.32 (t, J=6.0 Hz, 1H), 7.90 (d, J=2.5 Hz, 1H), 7.60 (d, J=9.0 Hz, 1H), 7.54 (d, J=7.5 Hz, 1H), 7.39 (t, J=3.0 Hz, 1H), 7.21 (d, J=2.5 Hz, 1H), 6.82 (t, J=7.5 Hz, 1H), 6.74 (d, J=9.0 Hz, 1H), 6.66 (t, J=8.5 Hz, 3H), 6.54 (d, J=7.5 Hz, 1H), 6.29 (dd, J=17.0, 2.0 Hz, 2H), 5.32 (t, J=4.5 Hz, 1H), 3.84 (dd, J=11.0, 3.0 Hz, 2H), 3.30-3.21 (m, 5H), 3.12-3.01 (m, 5H), 2.16-2.07 (m, 3H), 2.03-1.95 (m, 5H), 1.89-1.76 (m, 2H), 1.63-1.58 (m, 2H), 1.49-1.42 (m, 2H), 1.16-1.07 (m, 1H).

### Compound 41

¹H NMR (400 MHz, DMSO) δ 11.65 (s, 1H), 8.77 (s, 1H), 8.53 (d, J=2.1 Hz, 1H), 8.01 (d, J=2.6 Hz, 1H), 7.79 (d, J=9.3 Hz, 1H), 7.52 (d, J=8.9 Hz, 1H), 7.49-7.45 (m, 2H), 7.28 (dd, J=7.2, 2.1 Hz, 1H), 7.12-6.98 (m, 3H), 6.74-6.68 (m, 1H), 6.36 (dd, J=3.3, 1.8 Hz, 1H), 6.24 (d, J=2.0 Hz, 1H), 4.42 (s, 1H), 4.35 (t, J=5.1 Hz, 1H), 3.86 (d, J=8.0 Hz, 1H), 3.58 (d, J=7.0 Hz, 1H), 3.48-3.38 (m, 3H), 3.23 (d, J=6.0 Hz, 1H), 3.18-3.02 (m, 5H), 2.95-2.87 (m, 2H), 2.15-2.04 (m, 3H), 2.01-1.92 (m, 1H), 1.87-1.81 (m, 2H), 1.73-1.65 (m, 1H), 1.62-1.55 (m, 1H), 1.53-1.45 (m, 1H), 1.26-1.15 (m, 4H).

### Compound 42

¹H NMR (400 MHz, DMSO) δ 11.66 (s, 1H), 8.75 (s, 1H), 8.53 (d, J=2.0 Hz, 1H), 8.01 (d, J=2.8 Hz, 1H), 7.84-7.75 (m, 1H), 7.56-7.43 (m, 3H), 7.28 (dd, J=7.2, 2.4 Hz, 1H), 7.12-7.04 (m, 2H), 7.00 (d, J=9.2 Hz, 1H), 6.70 (dd, J=8.8, 2.0 Hz, 1H), 6.36 (dd, J=3.2, 2.0 Hz, 1H), 6.24 (d, J=2.0 Hz, 1H), 4.29 (s, 4H), 3.53-3.40 (m, 5H), 3.23-2.99 (m, 7H), 2.78-2.66 (m, 2H), 2.62-2.54 (m, 1H), 2.46-2.40 (m, 1H), 2.15-2.04 (m, 3H), 2.01-1.92 (m, 1H), 1.89-1.76 (m, 5H), 1.63-1.33 (m, 3H), 1.24-1.12 (m, 1H).

### Compound 43

¹H NMR (400 MHz, DMSO): δ 11.5 (s, 1H), 8.54 (s, 1H), 8.31-8.30 (m, 2H), 7.89 (s, 1H), 7.61-7.58 (dd, J=19.6 Hz, 9.2 Hz, 2H), 7.38 (s, 1H), 7.28-7.08 (m, 4H), 6.65 (s, 2H), 6.29 (m, 2H), 3.17-3.08 (m, 9H), 2.85-2.68 (m, 8H), 2.41 (s, 3H), 2.12 (s, 3H), 2.00-1.50 (m, 7H), 1.20 (s, 2H).

### Compound 44

¹H NMR (400 MHz, DMSO) δ 11.49 (s, 1H), 8.43-8.39 (m, 1H), 8.36 (d, J=2.1 Hz, 1H), 8.21 (s, 2H), 7.90 (d, J=2.5 Hz, 1H), 7.61 (d, J=8.7 Hz, 2H), 7.40 (s, 1H), 7.28 (d, J=9.2 Hz, 1H), 7.24 (d, J=2.6 Hz, 1H), 7.13-7.05 (m, 2H), 6.80 (d, J=9.4 Hz, 1H), 6.65 (d, J=8.8 Hz, 1H), 6.30-6.27 (m, 2H), 3.17-2.91 (m, 6H), 2.70-2.60 (m, 4H), 2.18-1.93 (m, 9H), 1.88-1.75 (m, 4H), 1.63-1.47 (m, 3H), 1.40-1.13 (m, 5H).

### Compound 45

¹H NMR (400 MHz, DMSO) δ 11.67 (s, 1H), 11.53 (s, 1H), 8.61 (t, J=6.0 Hz, 1H), 8.56 (d, J=2.4 Hz, 1H), 8.04 (d, J=2.4 Hz, 1H), 7.80 (dd, J=9.2, 2.0 Hz, 1H), 7.54-7.48 (m, 3H), 7.29-7.23 (m, 1H), 7.21-7.09 (m, 4H), 6.71 (dd, J=8.8, 2.0 Hz, 1H), 6.38 (dd, J=3.2, 2.0 Hz, 1H), 6.23 (d, J=2.0 Hz, 1H), 5.35-5.24 (m, 1H), 3.85 (dd, J=11.2, 3.2 Hz, 2H), 3.80 (s, 1H), 3.31-3.23 (m, 4H), 3.18-3.03 (m, 7H), 2.89 (s, 3H), 2.45-2.36 (m, 2H), 2.30-2.22 (m, 2H), 1.95-1.84 (m, 1H), 1.62 (d, J=12.4 Hz, 2H), 1.32-1.20 (m, 2H).

### Compound 46

¹H NMR (400 MHz, DMSO) δ 11.65 (s, 1H), 11.52 (s, 1H), 8.52 (s, 1H), 8.47 (s, 1H), 8.02 (d, J=2.2 Hz, 1H), 7.74 (d, J=9.0 Hz, 1H), 7.56-7.45 (m, 3H), 7.28 (dd, J=7.1, 2.1 Hz, 1H), 7.08 (q, J=7.2 Hz, 2H), 6.98 (s, 1H), 6.69 (d, J=8.8 Hz, 1H), 6.37 (s, 1H), 6.24 (d, J=1.7 Hz, 1H), 4.56 (s, 2H), 4.49 (s, 2H), 3.26-2.98 (m, 7H), 2.38-2.25 (m, 4H), 2.16-1.80 (m, 8H), 1.62-1.45 (m, 2H), 1.25-1.13 (m, 3H).

### Compound 47

¹H NMR (400 MHz, CDCl₃) δ 9.31 (s, 1H), 8.82 (d, J=1.9 Hz, 1H), 8.45 (s, 1H), 8.21-8.03 (m, 2H), 7.87 (t, J=10.7 Hz, 1H), 7.63 (s, 1H), 7.35 (d, J=8.1 Hz, 2H), 7.29 (t, J=7.3 Hz, 1H), 7.04-6.75 (m, 4H), 6.55-6.42 (m, 2H), 5.91 (s, 1H), 3.95 (dd, J=11.2, 3.6 Hz, 3H), 3.35 (t, J=10.9 Hz, 3H), 3.25-3.13 (m, 3H), 3.10-2.93 (m, 5H), 2.72-2.50 (m, 1H), 2.40 (d, J=6.8 Hz, 5H), 2.17-2.06 (m, 3H), 2.05-1.78 (m, 5H), 1.73-1.48 (m, 6H).

### Compound 48

¹H NMR (400 MHz, DMSO) δ 12.06 (s, 1H), 8.74-8.52 (m, 2H), 7.94 (dd, J=9.2, 2.2 Hz, 1H), 7.75 (s, 2H), 7.40-7.04 (m, 4H), 6.94 (s, 2H), 3.64-3.24 (m, 12H), 3.11 (s, 1H), 3.07-3.00 (m, 2H), 3.00-2.93 (m, 2H), 2.82 (s, 3H), 2.70-2.56 (m, 2H), 2.16 (s, 2H), 1.98-1.79 (m, 3H), 1.70-1.44 (m, 8H).

### Compound 49

¹H NMR (400 MHz, CDCl₃) δ 9.43 (s, 1H), 8.81 (t, J=8.9 Hz, 1H), 8.55-8.36 (m, 3H), 8.17-8.06 (m, 2H), 7.88 (t, J=12.8 Hz, 1H), 7.64 (d, J=2.4 Hz, 1H), 7.52-7.43 (m, 1H), 7.36 (dd, J=9.2, 6.3^{Hz}, 1H), 7.25 (dd, J=7.8, 4.7 Hz, 2H), 7.07-6.93 (m, 3H), 6.86 (t, J=10.1 Hz, 1H), 6.57-6.42 (m, 2H), 3.96 (dd, J=11.7, 3.3 Hz, 3H), 3.35 (dd, J=11.7, 10.2 Hz, 3H), 3.26-3.13 (m, 4H), 3.10-3.01 (m, 4H), 2.53-2.37 (m, 3H), 2.21-2.09 (m, 2H), 2.10-1.90 (m, 3H), 1.66 (t, J=12.8 Hz, 3H), 1.41-1.27 (m, 3H).

### Compound 50

¹H NMR (500 MHz, DMSO) δ 11.65 (s, 1H), 11.59-11.45 (s, 1H), 8.56-8.52 (m, 2H), 8.02 (s, 1H), 7.76 (d, J=8.1 Hz, 1H), 7.53 (d, J=8.8 Hz, 1H), 7.48 (s, 2H), 7.09-6.97 (m, 3H), 6.94-6.87 (m, 1H), 6.70 (d, J=7.5 Hz, 1H), 6.36 (s, 1H), 6.24 (s, 1H), 5.14 (s, 1H), 4.70 (s, 1H), 3.85 (d, J=9.1 Hz, 2H), 3.31-3.04 (m, 8H), 2.63 (d, J=11.8 Hz, 1H), 2.48-2.43 (m, 2H), 2.16-2.06 (m, 3H), 1.92 (s, 3H), 1.90-1.79 (m, 3H), 1.65-1.45 (m, 4H), 1.32-1.12 (m, 5H).

### Compound 51

¹H NMR (500 MHz, DMSO) δ 11.68 (s, 1H), 11.51 (s, 1H), 8.62 (t, J=5.5 Hz, 1H), 8.56 (d, J=2.0 Hz, 1H), 8.04 (d, J=2.5 Hz, 1H), 7.80 (d, J=9.0 Hz, 1H), 7.54-7.48 (m, 3H), 7.10 (d, J=8.0 Hz, 2H), 7.01 (t, J=7.5 Hz, 1H), 6.92 (d, J=7.5 Hz, 1H), 6.71 (dd, J=9.0, 1.5 Hz, 1H), 6.38 (dd, J=3.0, 2.0 Hz, 1H), 6.23 (d, J=1.5 Hz, 1H), 3.85 (dd, J=11.0, 3.0 Hz, 2H), 3.32-3.22 (m, 4H), 3.20-3.05 (m, 5H), 2.79 (dd, J=12.5, 7.0 Hz, 1H), 2.48-2.41 (m, 2H), 2.16-2.02 (m, 3H), 1.94-1.81 (m, 3H), 1.61 (d, J=12.0 Hz, 2H), 1.59-1.43 (m, 2H), 1.31-1.22 (m, 5H), 1.13 (dd, J=12.5, 6.5 Hz, 6H).

### Compound 52

¹H NMR (400 MHz, DMSO) δ 11.64 (s, 1H), 11.52 (s, 1H), 8.52 (s, 2H), 8.01 (s, 1H), 7.74 (s, 1H), 7.63-7.38 (m, 4H), 7.18-6.99 (m, 4H), 6.70 (d, J=10.1 Hz, 1H), 6.36 (s, 1H), 6.25 (s, 1H), 3.85 (d, J=8.0 Hz, 2H), 3.31-3.22 (m, 4H), 3.19-3.07 (m, 4H), 2.81-2.73 (m, 1H), 2.21-2.05 (m, 4H), 1.97-1.74 (m, 4H), 1.67-1.51 (m, 5H), 1.34-1.19 (m, 4H).

### Compound 53

¹H NMR (400 MHz, DMSO) δ 11.59 (s, 1H), 8.65-8.58 (m, 1H), 8.49 (d, J=1.8 Hz, 1H), 7.97 (d, J=2.5 Hz, 1H), 7.71 (d, J=9.2 Hz, 1H), 7.55 (d, J=8.8 Hz, 1H), 7.45-7.42 (m, 1H), 7.39 (s, 1H), 7.27 (d, J=7.3 Hz, 1H), 7.16-6.98 (m, 5H), 6.68 (d, J=8.8 Hz, 1H), 6.33 (s, 1H), 6.26 (s, 1H), 4.18-4.11 (m, 1H), 3.94-3.83 (m, 1H), 3.52-3.41 (m, 2H), 3.20-2.94 (m, 5H), 2.42-2.21 (m, 3H), 2.16-1.97 (m, 4H), 1.97-1.76 (m, 3H), 1.63-1.56 (m, 1H), 1.55-1.43 (m, 3H), 1.24-1.13 (m, 3H).

### Compound 54

¹H NMR (400 MHz, DMSO): δ 12.8 (s, 1H), 11.64 (s, 1H), 8.56-8.53 (m, 2H), 8.01 (d, J=2.4 Hz, 1H), 7.75-7.77 (m, 1H), 7.48-7.54 (m, 3H), 7.10-7.18 (m, 4H), 6.69 (d, J=2 Hz, 1H), 6.36 (d, J=4.8 Hz, 1H), 6.24 (d, J=8.4 Hz, 2H), 3.86 (dd, J=2.4 Hz, 11.2 HZ, 2H), 3.13-3.23 (m, 13H), 2.08-2.10 (m, 3H), 1.95 (m, 2H), 1.88 (m, 1H), 1.76 (m, 4H), 1.62 (m, 3H).

### Compound 55

¹H NMR (400 MHz, CDCl₃) δ 8.97 (s, 1H), 8.90 (d, J=2.2 Hz, 1H), 8.52 (s, 1H), 8.19 (t, J=4.7 Hz, 2H), 7.96 (d, J=9.1 Hz, 1H), 7.69 (s, 1H), 7.42 (s, 1H), 7.13 (d, J=7.2 Hz, 1H), 7.02-6.79 (m, 3H), 6.63-6.46 (m, 2H), 5.98 (s, 1H), 4.10-3.93 (m, 2H), 3.50-3.03 (m, 12H), 2.85-2.83 (m, 1H), 2.50-2.44 (m, 2H), 2.22-2.18 (m, 2H), 2.01-1.96 (m, 7H), 1.82-1.67 (m, 6H), 1.49-1.17 (m, 6H).

### Compound 56

¹H NMR (400 MHz, DMSO) δ 11.66 (s, 1H), 11.51 (s, 1H), 9.02 (s, 1H), 8.59 (s, 1H), 8.54 (s, 1H), 8.03 (d, J=2.0 Hz, 1H), 7.77 (d, J=8.5 Hz, 1H), 7.55-7.45 (m, 3H), 7.16-7.11 (m, 1H), 7.07 (d, J=5.0 Hz, 3H), 6.70 (dd, J=9.0, 2.0 Hz, 1H), 6.37 (d, J=1.0 Hz, 1H), 6.24 (d, J=1.5 Hz, 1H), 3.85 (dd, J=11.5, 3.0 Hz, 2H), 3.29-3.01 (m, 11H), 2.89 (s, 3H), 2.49-2.42 (m, 2H), 2.13-2.01 (m, 2H), 1.96-1.85 (m, 2H), 1.84-1.75 (m, 1H), 1.61 (d, J=11.5 Hz, 2H), 1.58-1.46 (m, 2H), 1.29-1.14 (m, 4H).

### Compound 57

¹H NMR (400 MHz, DMSO) δ 11.61 (s, 1H), 8.67 (s, 1H), 8.52 (s, 1H), 8.00 (s, 1H), 7.75 (d, J=6.1 Hz, 1H), 7.53 (d, J=8.9 Hz, 1H), 7.45 (s, 2H), 7.28 (dd, J=7.2, 1.9 Hz, 1H), 7.12-7.03 (m, 3H), 6.68 (d, J=8.9 Hz, 1H), 6.35 (s, 1H), 6.25 (s, 1H), 4.05 (dd, J=12.0, 4.9 Hz, 1H), 3.55-3.43 (m, 3H), 3.20-3.02 (m, 4H), 2.98-2.89 (m, 1H), 2.76-2.63 (m, 1H), 2.25 (s, 3H), 2.17-1.83 (m, 6H), 1.63-1.40 (m, 4H), 1.29-1.12 (m, 6H).

### Compound 58

¹H NMR (400 MHz, DMSO) δ 11.64 (s, 1H), 8.54 (d, J=15.3 Hz, 2H), 8.02 (s, 1H), 7.75 (d, J=7.2 Hz, 1H), 7.56-7.43 (m, 3H), 7.06 (s, 1H), 6.75-6.66 (m, 2H), 6.50 (d, J=7.7 Hz, 1H), 6.38-6.22 (m, 3H), 3.91-3.79 (m, 2H), 3.25-2.99 (m, 10H), 2.58 (dd, J=13.2, 7.0 Hz, 1H), 2.45-2.36 (m, 2H), 2.17-1.99 (m, 2H), 1.95-1.83 (m, 2H), 1.83-1.73 (m, 1H), 1.66-1.51 (m, 3H), 1.51-1.40 (m, 1H), 1.32-1.19 (m, 3H), 1.17-1.01 (m, 1H).

### Compound 59

¹H NMR (400 MHz, DMSO) δ 11.65 (s, 1H), 11.51 (s, 1H), 9.34 (s, 1H), 8.58 (s, 1H), 8.54 (s, 1H), 8.03 (d, J=1.5 Hz, 1H), 7.77 (d, J=8.5 Hz, 1H), 7.54-7.46 (m, 3H), 7.08 (d, J=7.5 Hz, 2H), 7.01 (t, J=7.5 Hz, 1H), 6.96 (d, J=7.5 Hz, 1H), 6.70 (dd, J=9.0, 1.5 Hz, 1H), 6.37 (s, 1H), 6.24 (d, J=1.5 Hz, 1H), 3.85 (dd, J=11.5, 3.0 Hz, 2H), 3.31-3.23 (m, 4H), 3.21-3.02 (m, 6H), 2.73-2.66 (m, 1H), 2.48-2.43 (m, 2H), 2.14-2.03 (m, 2H), 1.99 (s, 3H), 1.96-1.85 (m, 2H), 1.82-1.73 (m, 1H), 1.61 (d, J=11.5 Hz, 2H), 1.58-1.46 (m, 2H), 1.31-1.13 (m, 4H).

### Compound 60

¹H NMR (400 MHz, DMSO) δ 11.65 (s, 1H), 11.52 (s, 1H), 8.60 (s, 1H), 8.55 (s, 1H), 8.02 (s, 1H), 7.78 (d, J=8.7 Hz, 1H), 7.56-7.43 (m, 3H), 7.23-7.03 (m, 5H), 6.72 (d, J=9.1 Hz, 1H), 6.37 (s, 1H), 6.26 (s, 1H), 4.06-3.72 (m, 5H), 3.63-3.53 (m, 1H), 3.30-3.07 (m, 4H), 3.00 (d, J=14.5 Hz, 1H), 2.81 (s, 3H), 2.76-2.60 (m, 5H), 2.36-2.30 (m, 1H), 2.22-2.09 (m, 3H), 1.95-1.84 (m, 1H), 1.61 (d, J=14.1 Hz, 2H), 1.32-1.16 (m, 3H).

### Compound 61A

¹H NMR (400 MHz, CDCl₃) δ 8.99 (s, 1H), 8.89 (d, J=2.0 Hz, 1H), 8.52 (t, J=5.0 Hz, 1H), 8.19 (s, 1H), 8.17 (s, 1H), 7.97 (d, J=9.0 Hz, 1H), 7.69 (s, 1H), 7.42 (s, 1H), 7.09 (d, J=7.0 Hz, 1H), 7.03 (t, J=7.5 Hz, 1H), 6.94-6.89 (m, 2H), 6.57 (d, J=9.0 Hz, 1H), 6.53 (s, 1H), 5.99 (s, 1H), 4.03 (dd, J=11.5, 3.5 Hz, 2H), 3.77-3.71 (m, 1H), 3.67-3.58 (m, 1H), 3.46-3.37 (m, 3H), 3.37-3.30 (m, 1H), 3.27 (t, J=6.0 Hz, 2H), 3.17 (d, J=5.5 Hz, 1H), 3.14-3.08 (m, 4H), 2.82 (dd, J=13.0, 7.0 Hz, 1H), 2.53-2.45 (m, 2H), 2.21-2.15 (m, 2H), 2.09-2.02 (m, 1H), 1.99-1.92 (m, 1H), 1.92-1.85 (m, 1H), 1.74 (d, J=12.0 Hz, 2H), 1.49-1.39 (m, 3H), 1.35-1.25 (m, 3H), 1.22 (d, J=7.0 Hz, 3H).

### Compound 61B

¹H NMR (400 MHz, CDCl₃) δ 8.93 (s, 1H), 8.90 (d, J=2.0 Hz, 1H), 8.52 (t, J=5.5 Hz, 1H), 8.19 (d, J=2.5 Hz, 1H), 8.17 (d, J=2.0 Hz, 1H), 7.97 (d, J=9.0 Hz, 1H), 7.70 (d, J=2.5 Hz, 1H), 7.44-7.40 (m, 1H), 7.08 (d, J=7.0 Hz, 1H), 7.03 (t, J=7.5 Hz, 1H), 6.95-6.90 (m, 2H), 6.57 (dd, J=9.0, 2.0 Hz, 1H), 6.54-6.51 (m, 1H), 5.98 (d, J=2.0 Hz, 1H), 4.03 (dd, J=11.5, 4.0 Hz, 2H), 3.77-3.71 (m, 1H), 3.69-3.61 (m, 1H), 3.47-3.39 (m, 3H), 3.35-3.30 (m, 1H), 3.29-3.25 (m, 2H), 3.17 (d, J=5.5 Hz, 1H), 3.13-3.06 (m, 4H), 2.83 (dd, J=14.0, 7.0 Hz, 1H), 2.53-2.44 (m, 2H), 2.22-2.15 (m, 2H), 2.09-2.02 (m, 1H), 2.00-1.93 (m, 1H), 1.91-1.86 (m, 1H), 1.74 (d, J=11.5 Hz, 2H), 1.49-1.39 (m, 3H), 1.34-1.25 (m, 3H), 1.21 (d, J=7.0 Hz, 3H).

### Compound 62

¹H NMR (400 MHz, DMSO) δ 8.57 (d, J=2.3 Hz, 1H), 8.04 (d, J=2.6 Hz, 1H), 7.81 (dd, J=9.3, 2.3 Hz, 1H), 7.70 (dd, J=11.6, 7.9 Hz, 1H), 7.61-7.55 (m, 3H), 7.50 (d, J=3.4 Hz, 1H), 7.44 (dd, J=7.7, 5.7 Hz, 1H), 7.10 (d, J=9.5 Hz, 1H), 6.82 (d, J=7.2 Hz, 1H), 6.42 (d, J=3.4 Hz, 1H), 6.35 (s, 1H), 4.78-4.70 (m, 1H), 3.39-3.14 (m, 14H), 2.20-1.82 (m, 8H), 1.75-1.58 (m, 3H), 1.54-1.19 (m, 6H), 1.02-0.90 (m, 7H).

### Compound 63

¹H NMR (500 MHz, DMSO) δ 11.65 (s, 1H), 11.51 (s, 1H), 8.53 (s, 2H), 8.02 (s, 1H), 7.77 (s, 1H), 7.57-7.4 (m, 4H), 7.32 (d, J=7.4 Hz, 1H), 7.23-7.12 (m, 1H), 7.06 (s, 1H), 6.70 (d, J=8.8 Hz, 1H), 6.37 (s, 1H), 6.24 (s, 1H), 3.85 (d, J=9.4 Hz, 2H), 3.55 (s, 1H), 3.28-3.07 (m, 9H), 2.16-1.82 (m, 7H), 1.76-1.48 (m, 11H), 1.33-1.18 (m, 4H).

### Compound 64

¹H NMR (400 MHz, DMSO): 11.34 (s, 1H), 8.53-8.45 (m, 2H), 8.01 (s, 1H), 7.79-7.77 (m, 4H), 7.55-7.47 (m, 3H), 7.28-7.27 (m, 1H), 7.26-7.05 (m, 3H) 6.70-6.68 (m, 1H), 6.36 (s, 1H). 6.24 (s, 1H), 5.62-5.61 (m, 1H), 3.89-3.75 (m, 3H), 3.53-3.74 (m, 2H), 3.39-3.02 (m, 7H), 2.88-2.78 (m, 2H), 23.32-2.01 (m, 3H), 1.98-1.78 (m, 5H), 1.76-1.35 (m, 6H).

### Compound 65

¹H NMR (500 MHz, DMSO) δ 11.69 (s, 1H), 11.46 (s, 1H), 8.82 (t, J=4.4 Hz, 1H), 8.57 (d, J=2.2 Hz, 1H), 8.04 (d, J=2.6 Hz, 1H), 7.82 (dd, J=9.1, 1.9 Hz, 1H), 7.55-7.48 (m, 3H), 7.45 (d, J=7.9 Hz, 1H), 7.13 (d, J=7.1 Hz, 1H), 7.04 (d, J=9.3 Hz, 1H), 6.99 (t, J=7.7 Hz, 1H), 6.74-6.68 (m, 1H), 6.38 (dd, J=3.2, 1.8 Hz, 1H), 6.23 (d, J=1.9 Hz, 1H), 3.53-3.42 (m, 7H), 3.25-3.08 (m, 5H), 3.06-2.99 (m, 1H), 2.74-2.65 (m, 2H), 2.48-2.42 (m, 5H), 2.16-2.05 (m, 3H), 2.00 (s, 3H), 1.96-1.88 (m, 1H), 1.82 (s, 1H), 1.63-1.44 (m, 2H), 1.25-1.11 (m, 2H).

### Compound 66

¹H NMR (400 MHz, DMSO) δ 11.70-11.55 (m, 2H), 8.73-8.47 (m, 2H), 8.05 (d, J=2.5 Hz, 1H), 7.81 (dd, J=9.3, 1.9 Hz, 1H), 7.61-7.38 (m, 4H), 7.12 (d, J=7.6 Hz, 2H), 6.99 (t, J=7.7 Hz, 1H), 6.71 (dd, J=9.0, 1.9 Hz, 1H), 6.39 (dd, J=3.2, 1.8 Hz, 1H), 6.22 (d, J=1.8 Hz, 1H), 3.85 (dd, J=11.3, 3.0 Hz, 2H), 3.49 (t, J=12.3 Hz, 1H), 3.32-2.96 (m, 10H), 2.09 (d, J=5.3 Hz, 3H), 1.99-1.76 (m, 3H), 1.70-1.44 (m, 4H), 1.37-1.09 (m, 5H).

### Compound 67

¹H NMR (400 MHz, DMSO): 11.65 (s, 1H), 8.13-8.01 (m, 2H), 7.53 (s, 1H), 7.51 (br, 1H), 7.49-7.47 (m, 4H), 7.13 (d, J=7.2, 1H), 7.01-6.97 (m, 2H) 6.69 (d, J=8, 1H), 6.36 (s, 1H). 6.24 (s, 1H), 4.28 (s, 2H), 4.18 (s, 2H), 3.46-3.33 (m, 1H), 3.31-3.02 (m, 6H), 1.67-1.16 (m, 13H), 0.93-0.86 (m, 2H).

### Compound 68

¹H NMR (400 MHz, DMSO) δ 11.66 (s, 1H), 8.75 (s, 1H), 8.53 (s, 1H), 8.01 (d, J=2.5 Hz, 1H), 7.79 (d, J=8.3 Hz, 1H), 7.59-7.40 (m, 4H), 7.13 (d, J=6.8 Hz, 1H), 6.99 (t, J=7.7 Hz, 2H), 6.70 (d, J=11.1 Hz, 1H), 6.36 (s, 1H), 6.24 (s, 1H), 4.29 (s, 4H), 3.50 (s, 3H), 3.25-2.97 (m, 7H), 2.67 (s, 2H), 2.11 (s, 3H), 1.95-1.80 (m, 7H), 1.55-1.48 (m, 3H), 1.31-1.11 (m, 5H).

### Compound 69

¹H NMR (400 MHz, DMSO) δ 11.55 (s, 1H), 8.43 (d, J=17.4 Hz, 2H), 7.94 (d, J=2.4 Hz, 1H), 7.62 (dd, J=18.3, 9.4 Hz, 2H), 7.50-7.39 (m, 2H), 7.30 (s, 1H), 7.15 (d, J=7.6 Hz, 1H), 7.00 (t, J=7.8 Hz, 1H), 6.88 (d, J=10.0 Hz, 1H), 6.67 (d, J=9.3 Hz, 1H), 6.30 (d, J=12.1 Hz, 2H), 3.55-3.45 (m, 2H), 3.10-3.05 (m, 6H), 2.83-2.60 (m, 5H), 2.22-1.73 (m, 10H), 1.65-1.15 (m, 9H).

### Compound 70

¹H NMR (400 MHz, DMSO) δ 11.64 (s, 1H), 8.52 (s, 2H), 8.14 (s, 1H), 8.01 (s, 1H), 7.73 (d, J=9.3 Hz, 1H), 7.54 (d, J=8.8 Hz, 1H), 7.49-7.41 (m, 2H), 7.28 (dd, J=7.2, 2.0 Hz, 1H), 7.14-6.98 (m, 3H), 6.69 (d, J=8.9 Hz, 1H), 6.35 (s, 1H), 6.25 (s, 1H), 3.85 (d, J=7.8 Hz, 2H), 3.35-3.23 (m, 6H), 3.17-3.03 (m, 5H), 2.16-1.80 (m, 7H), 1.69-1.56 (m, 3H), 1.49 (t, J=12.8 Hz, 1H), 1.32-1.11 (m, 4H).

### Compound 71

¹H NMR (400 MHz, DMSO) δ 11.63 (s, 1H), 8.50 (s, 2H), 8.14 (s, 1H), 8.01 (s, 1H), 7.73 (s, 1H), 7.59-7.43 (m, 3H), 7.28 (dd, J=7.2, 2.0 Hz, 1H), 7.13-6.97 (m, 3H), 6.69 (d, J=8.9 Hz, 1^{H}) 6.35 (s, 1H), 6.24 (s, 1H), 4.29 (s, 2H), 4.19 (s, 2H), 3.45-3.38 (m, 1H), 3.25-3.17 (m, 4H), 3.15-3.01 (m, 4H), 2.15-2.00 (m, 5H), 1.98-1.91 (m, 1H), 1.89-1.77 (m, 1H), 1.69-1.46 (m, 6H), 1.37 (t, J=11.5 Hz, 2H), 1.26-1.12 (m, 2H), 1.08-0.94 (m, 2H).

### Compound 72

¹H NMR (400 MHz, DMSO) δ 11.67 (s, 1H), 11.53 (s, 1H), 8.53 (s, 2H), 8.14 (s, 1H), 8.03 (d, J=1.9 Hz, 1H), 7.76 (d, J=7.3 Hz, 1H), 7.55-7.44 (m, 3H), 7.28 (dd, J=7.1, 2.2 Hz, 1H), 7.12-6.97 (m, 3H), 6.70 (d, J=9.0 Hz, 1H), 6.37 (s, 1H), 6.23 (s, 1H), 4.56 (s, 2H), 4.49 (s, 2H), 3.41 (d, J=12.1 Hz, 1H), 3.23 (d, J=5.5 Hz, 1H), 3.17-3.02 (m, 6H), 2.41-2.25 (m, 4H), 2.15-2.04 (m, 3H), 2.01-1.81 (m, 5H), 1.63-1.53 (m, 1H), 1.54-1.42 (m, 1H), 1.25-1.05 (m, 2H).

### Compound 73

¹H NMR (400 MHz, DMSO) δ 11.66 (s, 1H), 11.57 (s, 1H), 8.65-8.48 (m, 2H), 8.02 (s, 1H), 7.75 (d, J=10.2 Hz, 1H), 7.62-7.41 (m, 5H), 7.30 (t, J=7.8 Hz, 1H), 7.05 (s, 1H), 6.70 (d, J=9.0 Hz, 1H), 6.37 (s, 1H), 6.24 (s, 1H), 3.92-3.81 (m, 2H), 3.41-3.07 (m, 10H), 2.2-1.79 (m, 7H), 1.66-1.49 (m, 5H), 1.38-1.20 (m, 4H).

### Compound 74

¹H NMR (400 MHz, DMSO) δ 11.57 (s, 1H), 8.42 (s, 2H), 8.16 (s, 1H), 7.95 (s, 1H), 7.69-7.54 (m, 3H), 7.50-7.39 (m, 2H), 7.38-7.27 (m, 2H), 6.85 (s, 1H), 6.67 (d, J=8.3 Hz, 1H), 6.29 (d, J=18.2 Hz, 2H), 4.29 (s, 2H), 4.18 (s, 2H), 3.39-3.07 (m, 8H), 2.21-1.81 (m, 8H), 1.71-1.46 (m, 6H), 1.43-1.25 (m, 4H), 1.03-0.88 (m, 2H).

### Compound 75

¹H NMR (400 MHz, DMSO) δ 11.67 (s, 1H), 8.52 (s, 2H), 8.02 (s, 1H), 7.81-7.14 (m, 8H), 6.98 (s, 1H), 6.69 (s, 1H), 6.27-6.23 (m, 2H), 4.56-4.42 (m, 4H), 3.27-2.90 (m, 7H), 2.33-1.74 (m, 12H), 1.72-1.43 (m, 3H), 1.38-1.12 (m, 2H).

### Compound 76

¹H NMR (400 MHz, DMSO): 11.56 (s, 1H), 8.40 (br, 2H), 8.17 (s, 1H), 7.95 (br, 1H), 7.62-7.58 (m, 4H), 7.42-7.23 (m, 8H), 6.75-6.72 (br, 1H) 6.67 (d, J=8.4, 1H), 6.31 (s, 1H). 6.27 (s, 1H), 3.85-3.82 (m, 2H), 3.26-3.05 (m, 11H), 2.33 (br, 4H), 2.14 (br, 4H), 1.62-1.59 (m, 5H), 1.26-1.23 (m, 3H).

### Compound 77

¹H NMR (400 MHz, DMSO) δ 11.53 (s, 1H), 8.35 (s, 1H), 8.24 (s, 2H), 7.91 (s, 1H), 7.63-7.36 (m, 4H), 7.26-6.97 (m, 3H), 6.67 (t, J=8.1 Hz, 2H), 6.29 (d, J=7.1 Hz, 2H), 4.52 (d, J=28.3 Hz, 4H), 3.34-3.18 (m, 4H), 3.15-2.98 (m, 4H), 2.42-2.26 (m, 3H), 2.18-2.05 (m, 3H), 2.02-1.75 (m, 5H), 1.64-1.43 (m, 2H), 1.29-1.15 (m, 3H).

### Compound 78

¹H NMR (400 MHz, DMSO) δ 11.70 (s, 1H), 11.54 (s, 1H), 8.63 (t, J=5.8 Hz, 1H), 8.57 (d, J=2.1 Hz, 1H), 8.05 (d, J=2.5 Hz, 1H), 7.80 (dd, J=9.2, 1.7 Hz, 1H), 7.58-7.47 (m, 3H), 7.40 (d, J=7.3 Hz, 1H), 7.18-7.07 (m, 2H), 6.71 (dd, J=8.8, 1.9 Hz, 1H), 6.39 (dd, J=3.1, 1.7 Hz, 1H), 6.21 (d, J=1.7 Hz, 1H), 3.85 (dd, J=11.4, 2.8 Hz, 2H), 3.33-3.22 (m, 6H), 3.21-3.05 (m, 5H), 2.48-2.41 (m, 1H), 2.13-1.98 (m, 3H), 1.97-1.84 (m, 2H), 1.83-1.72 (m, 1H), 1.68-1.45 (m, 4H), 1.32-1.17 (m, 4H).

### Compound 79

¹H NMR (400 MHz, DMSO) δ 11.69 (s, 1H), 11.60 (s, 1H), 8.59 (t, J=6.1 Hz, 1H), 8.54 (d, J=1.9 Hz, 1H), 8.04 (d, J=2.5 Hz, 1H), 7.77 (d, J=9.2 Hz, 1H), 7.55-7.47 (m, 3H), 7.41 (d, J=7.2 Hz, 1H), 7.16 (d, J=9.8 Hz, 1H), 7.05 (d, J=8.9 Hz, 1H), 6.70 (dd, J=9.0, 1.7 Hz, 1H), 6.40-6.36 (m, 1H), 6.21 (d, J=1.6 Hz, 1H), 4.29 (s, 2H), 4.19 (s, 2H), 3.34-3.17 (m, 6H), 3.16-3.02 (m, 3H), 2.48-2.41 (m, 1H), 2.14-1.97 (m, 5H), 1.97-1.86 (m, 1H), 1.83-1.74 (m, 1H), 1.71-1.43 (m, 6H), 1.42-1.32 (m, 2H), 1.31-1.18 (m, 1H), 1.03-0.87 (m, 2H).

### Compound 80

¹H NMR (400 MHz, DMSO) δ 11.67 (s, 1H), 8.75 (s, 1H), 8.52 (d, J=2.1 Hz, 1H), 8.01 (d, J=2.6 Hz, 1H), 7.78 (dd, J=9.1, 1.8 Hz, 1H), 7.52 (d, J=8.9 Hz, 1H), 7.47 (t, J=2.7 Hz, 2H), 7.41 (d, J=7.3 Hz, 1H), 7.16 (d, J=9.9 Hz, 1H), 6.98 (d, J=9.3 Hz, 1H), 6.69 (dd, J=8.9, 2.1 Hz, 1H), 6.36 (dd, J=3.3, 1.8 Hz, 1H), 6.23 (d, J=2.1 Hz, 1H), 4.29 (s, 4H), 3.53-3.45 (m, 2H), 3.28-3.05 (m, 9H), 2.72-2.62 (m, 2H), 2.47-2.42 (m, 2H), 2.16-1.91 (m, 5H), 1.89-1.74 (m, 5H), 1.62-1.46 (m, 2H), 1.32-1.19 (m, 2H).

### Compound 81

¹H NMR (400 MHz, DMSO) δ 11.54 (s, 1H), 8.40 (s, 1H), 8.30-8.15 (m, 2H), 7.92 (d, J=2.3 Hz, 1H), 7.68-7.55 (m, 2H), 7.47-7.39 (m, 2H), 7.27 (s, 1H), 7.18 (d, J=9.7 Hz, 1H), 6.85 (d, J=7.7 Hz, 1H), 6.67 (d, J=8.8 Hz, 1H), 6.33-6.25 (m, 2H), 5.62 (d, J=4.9 Hz, 1H), 3.87 (t, J=7.8 Hz, 1H), 3.81-3.73 (m, 2H), 3.54-3.40 (m, 5H), 3.22-3.03 (m, 4H), 2.92-2.82 (m, 1H), 2.70-2.63 (m, 1H), 2.14-1.74 (m, 8H), 1.64-1.47 (m, 3H), 1.28-1.17 (m, 3H).

### Compound 82

¹H NMR (400 MHz, DMSO) δ 11.59 (s, 1H), 8.45 (s, 1H), 8.33 (s, 1H), 8.15 (s, 1H), 7.96 (s, 1H), 7.68 (d, J=8.7 Hz, 1H), 7.56 (d, J=8.7 Hz, 1H), 7.47-7.30 (m, 3H), 7.17 (d, J=9.8 Hz, 1H), 6.93 (s, 1H), 6.67 (d, J=8.7 Hz, 1H), 6.32 (s, 1H), 6.27 (s, 1H) 5.65-5.60 (m, 1H), 3.87 (t, J=7.8 Hz, 1H), 3.80-3.72 (m, 2H), 3.55-3.43 (m, 3H), 3.29-3.03 (m, 7H), 2.92-2.83 (m, 1H), 2.71-2.61 (m, 1H), 2.16-1.87 (m, 6H), 1.86-1.72 (m, 2H), 1.64-1.45 (m, 2H), 1.30-1.18 (m, 3H).

### Compound 83

¹H NMR (400 MHz, CDCl₃) δ 10.11 (s, 1H), 9.84 (s, 1H), 8.90 (d, J=2.1 Hz, 1H), 8.52 (t, J=5.4 Hz, 1H), 8.17 (s, 1H), 8.14-8.05 (m, 2H), 7.96 (d, J=9.1 Hz, 1H), 7.74 (d, J=2.4 Hz, 1H), 7.50-7.43 (m, 1H), 7.22 (dd, J=6.6, 2.7 Hz, 1H), 6.95-6.92 (m, 2H), 6.87 (d, J=9.2 Hz, 1H), 6.61-6.49 (m, 2H), 5.98 (d, J=2.0 Hz, 1H), 4.86-4.77 (m, 2H), 4.70 (t, J=6.8 Hz, 2H), 3.70 (s, 1H), 3.40-3.23 (m, 3H), 3.21-3.08 (m, 6H), 3.07-2.98 (m, 2H), 2.56-2.45 (m, 2H), 2.24-2.04 (m, 5H), 1.97-1.79 (m, 4H), 1.73-1.57 (m, 4H), 1.34-1.21 (m, 2H).

### Compound 84

¹H NMR (400 MHz, CDCl3) δ 9.86 (s, 1H), 8.95 (t, J=3.9 Hz, 1H), 8.89 (d, J=2.2 Hz, 1H), 8.22-8.13 (m, 3H), 7.97 (d, J=9.1 Hz, 1H), 7.75 (d, J=2.4 Hz, 1H), 7.47-7.43 (m, 1H), 7.22 (dd, J=6.8, 2.5 Hz, 1H), 6.97-6.91 (m, 2H), 6.88 (d, J=9.2 Hz, 1H), 6.60-6.51 (m, 2H), 5.97 (d, J=2.0 Hz, 1H), 4.71-4.62 (m, 4H), 3.60-3.54 (m, 1H), 3.45-3.30 (m, 3H), 3.17-3.08 (m, 6H), 2.78 (t, J=6.0 Hz, 3H), 2.69-2.39 (m, 7H), 2.21-1.83 (m, 6H), 1.68-1.54 (m, 2H), 1.33-1.21 (m, 2H).

### Compound 85

¹H NMR (400 MHz, CDCl3) δ 10.08 (s, 1H), 9.52 (s, 1H), 8.88 (s, 1H), 8.74 (t, J=3.3 Hz, 1H), 8.17-8.03 (m, 3H), 7.94 (d, J=9.2 Hz, 1H), 7.71 (d, J=2.3 Hz, 1H), 7.48-7.42 (m, 1H), 7.22 (d, J=8.8 Hz, 1H), 6.98-6.89 (m, 3H), 6.61-6.50 (m, 2H), 5.99 (s, 1H), 4.77-4.67 (m, 4H), 3.67-3.60 (m, 1H), 3.41-3.29 (m, 3H), 3.21-3.08 (m, 7H), 2.76-2.62 (m, 2H), 2.54-2.44 (m, 2H), 2.39-2.29 (m, 2H), 2.20-1.93 (m, 6H), 1.68-1.54 (m, 2H), 1.36-1.18 (m, 3H).

### Compound 86

¹H NMR (400 MHz, DMSO) δ 11.55 (s, 1H), 8.44 (t, J=5.3 Hz, 1H), 8.40 (d, J=1.6 Hz, 1H), 7.93 (d, J=2.5 Hz, 1H), 7.67-7.58 (m, 2H), 7.43 (t, J=2.6 Hz, 1H), 7.29 (s, 1H), 7.25 (dd, J=8.6, 2.6 Hz, 1H), 7.07 (dd, J=9.6, 2.4 Hz, 1H), 6.87 (d, J=9.3 Hz, 1H), 6.66 (d, J=8.2 Hz, 1H), 6.30 (d, J=11.7 Hz, 2H), 3.46-3.39 (m, 2H), 3.32-3.22 (m, 6H), 3.13-3.05 (m, 4H), 3.04-2.95 (m, 1H), 2.77-2.66 (m, 1H), 2.61 (s, 3H), 2.19-2.11 (m, 2H), 2.09-2.01 (m, 1H), 1.96-1.91 (m, 1H), 1.89-1.72 (m, 4H), 1.63-1.48 (m, 2H), 1.42-1.31 (m, 2H), 1.26-1.12 (m, 2H).

### Compound 87

¹H NMR (400 MHz, DMSO) δ 11.68 (s, 1H), 8.60 (t, J=5.8 Hz, 1H), 8.54 (d, J=1.7 Hz, 1H), 8.03 (d, J=2.4 Hz, 1H), 7.77 (d, J=9.2 Hz, 1H), 7.56-7.46 (m, 3H), 7.25 (dd, J=8.5, 2.6 Hz, 1H), 7.11-7.01 (m, 2H), 6.71 (d, J=7.5 Hz, 1H), 6.37 (d, J=2.7 Hz, 1H), 6.23 (d, J=1.4 Hz, 1H), 3.85 (dd, J=11.3, 3.1 Hz, 2H), 3.33-3.22 (m, 6H), 3.18-3.07 (m, 4H), 3.04-2.95 (m, 1H), 2.18-1.76 (m, 7H), 1.66-1.48 (m, 4H), 1.32-1.12 (m, 4H).

### Compound 88

¹H NMR (400 MHz, DMSO) δ 11.66 (s, 1H), 8.62 (t, J=4.3 Hz, 1H), 8.52 (d, J=2.0 Hz, 1H), 8.02 (d, J=2.5 Hz, 1H), 7.75 (d, J=9.1 Hz, 1H), 7.54 (d, J=8.8 Hz, 1H), 7.48 (d, J=2.6 Hz, 2H), 7.25 (dd, J=8.5, 2.6 Hz, 1H), 7.11-7.02 (m, 2H), 6.70 (d, J=7.1 Hz, 1H), 6.37 (s, 1H), 6.24 (s, 1H), 4.85 (s, 1H), 4.54 (t, J=6.5 Hz, 2H), 4.43 (t, J=6.1 Hz, 2H), 3.52-3.41 (m, 2H), 3.32-3.22 (m, 3H), 3.18-3.07 (m, 4H), 3.05-2.95 (m, 1H), 2.49-2.42 (m, 2H), 2.24-1.76 (m, 8H), 1.64-1.48 (m, 6H), 1.29-1.07 (m, 2H).

### Compound 89

¹H NMR (500 MHz, DMSO) δ 11.69 (s, 1H), 11.54 (s, 1H), 8.62 (t, J=5.7 Hz, 1H), 8.57 (d, J=2.2 Hz, 1H), 8.05 (d, J=2.5 Hz, 1H), 7.80 (dd, J=9.2, 1.8 Hz, 1H), 7.56-7.47 (m, 3H), 7.13-7.05 (m, 2H), 6.99 (t, J=9.0 Hz, 1H), 6.94 (d, J=7.2 Hz, 1H), 6.70 (dd, J=9.0, 2.0 Hz, 1H), 6.38 (dd, J=3.2, 1.8 Hz, 1H), 6.22 (d, J=1.9 Hz, 1H), 3.85 (dd, J=11.2, 2.8 Hz, 2H), 3.34-3.20 (m, 6H), 3.17-3.03 (m, 4H), 2.86 (dd, J=13.0, 6.5 Hz, 1H), 2.46 (s, 2H), 2.14-2.04 (m, 3H), 2.00-1.95 (m, 1H), 1.92-1.80 (m, 2H), 1.66-1.56 (m, 3H), 1.48 (t, J=13.7 Hz, 1H), 1.32-1.14 (m, 3H).

### Compound 90

¹H NMR (400 MHz, DMSO) δ 11.70 (s, 1H), 11.53 (s, 1H), 8.63 (t, J=5.7 Hz, 1H), 8.57 (d, J=2.2 Hz, 1H), 8.05 (d, J=2.6 Hz, 1H), 7.80 (dd, J=9.3, 2.0 Hz, 1H), 7.55-7.48 (m, 3H), 7.12 (d, J=9.4 Hz, 1H), 7.03-6.97 (m, 1H), 6.94-6.89 (m, 2H), 6.71 (d, J=9.0 Hz, 1H), 6.38 (dd, J=3.2, 1.8 Hz, 1H), 6.23 (d, J=1.9 Hz, 1H), 3.88-3.81 (m, 2H), 3.33-3.05 (m, 10H), 2.69 (dd, J=12.7, 6.4 Hz, 1H), 2.48-2.40 (m, 1H), 2.22 (s, 3H), 2.15-1.79 (m, 6H), 1.65-1.45 (m, 4H), 1.31-1.13 (m, 4H).

### Compound 91

¹H NMR (400 MHz, DMSO) δ 11.68 (s, 1H), 8.54 (s, 2H), 8.04 (s, 1H), 7.79 (d, J=9.5 Hz, 1H), 7.62-7.44 (m, 3H), 7.33 (d, J=7.1 Hz, 1H), 7.17-6.98 (m, 4H), 6.40 (s, 1H), 6.15 (d, J=7.1 Hz, 1H), 5.62 (s, 1H), 3.85 (d, J=8.5 Hz, 2H), 3.68 (d, J=6.6 Hz, 4H), 3.32-3.21 (m, 5H), 2.84-2.81 (m, 2H), 2.71-2.68 (m, 2H), 1.98-1.94 (m, 6H), 1.66-1.60 (m, 3H), 1.38-1.17 (m, 4H).

### Compound 92

¹H NMR (400 MHz, DMSO) δ 11.70 (s, 1H), 8.56 (s, 2H), 8.05 (s, 1H), 7.82 (d, J=8.4 Hz, 1H), 7.68-7.43 (m, 4H), 7.10 (d, J=6.4 Hz, 3H), 7.03 (d, J=6.6 Hz, 1H), 6.41 (s, 1H), 6.14 (d, J=8.4 Hz, 1H), 5.59 (s, 1H), 3.93-3.67 (m, 3H), 2.69-2.64 (d, J=21.1 Hz, 3H), 2.40 (s, 4H), 1.85-1.81 (m, 5H), 1.74-1.44 (m, 10H), 1.28-1.24 (m, 5H).

### Compound 93

¹H NMR (500 MHz, DMSO) δ 11.63 (s, 1H), 8.49 (s, 1H), 8.00 (s, 1H), 7.72 (d, J=8.0 Hz, 1H), 7.53 (d, J=9.0 Hz, 1H), 7.45 (d, J=19.0 Hz, 2H), 7.14 (s, 1H), 7.09 (s, 2H), 6.99 (s, 1H), 6.71 (d, J=7.0 Hz, 1H), 3.84 (d, J=8.0 Hz, 2H), 3.27 (dd, J=16.0, 9.0 Hz, 7H), 3.11 (s, 5H), 3.04-2.90 (m, 2H), 2.67 (d, J=30.0 Hz, 1H), 1.88 (s, 2H), 1.70 (s, 5H), 1.61 (d, J=12.0 Hz, 3H), 1.45 (s, 2H), 1.34-1.11 (m, 3H).

### Compound 94

¹H NMR (500 MHz, DMSO) δ 11.57 (s, 1H), 8.44 (s, 2H), 7.96 (s, 1H), 7.66 (d, J=9.0 Hz, 1H), 7.56 (d, J=9.0 Hz, 1H), 7.44 (s, 1H), 7.35-7.30 (m, 3H), 7.15 (d, J=7.0 Hz, 2H), 6.91 (s, 1H), 6.70 (d, J=7.0 Hz, 1H), 6.35-6.28 (m, 2H), 3.86-3.82 (m, 2H), 3.28-2.24 (m, 7H), 3.09 (s, 4H), 2.83-2.80 (m, 1H), 2.75-2.63 (m, 1H), 1.75-1.65 (m, 12H), 1.30-1.20 (m, 3H).

### Compound 95

¹H NMR (400 MHz, DMSO) δ 11.70 (s, 1H), 8.54 (s, 2H), 8.04 (s, 1H), 7.80 (s, 1H), 7.52 (d, J=8.8 Hz, 3H), 7.15-7.12 (m, 6H), 6.40 (s, 1H), 6.16 (d, J=8.8 Hz, 1H), 5.62 (s, 1H), 3.85 (s, 7H), 3.31-3.18 (m, 6H), 2.98 (s, 1H), 2.67 (s, 1H), 1.87 (s, 2H), 1.58-1.54 (m, 8H), 1.25 (d, J=11.6 Hz, 3H).

### Compound 96

¹H NMR (400 MHz, DMSO) δ 11.63 (s, 1H), 8.48 (s, 2H), 7.99 (s, 1H), 7.71 (d, J=8.3 Hz, 1H), 7.55-7.45 (m, 3H), 7.15-7.10 (m, 5H), 6.36 (s, 1H), 6.15 (d, J=8.1 Hz, 1H), 5.67 (s, 1H), 3.84 (d, J=10.1 Hz, 7H), 3.49 (s, 2H), 3.27 (dd, J=13.7, 9.0 Hz, 6H), 2.76 (s, 1H), 2.68 (d, J=10.9 Hz, 1H), 1.88 (s, 3H), 1.61 (d, J=11.1 Hz, 4H), 1.33-1.15 (m, 2H).

### Compound 97

¹H NMR (500 MHz, DMSO) δ 11.72 (s, 1H), 11.28 (s, 0.62H), 8.58 (d, J=10.0 Hz, 2H), 8.07 (s, 1H), 7.83 (d, J=8.5 Hz, 1H), 7.61 (s, 1H), 7.52 (d, J=8.5 Hz, 2H), 7.13 (d, J=9.0 Hz, 1H), 7.05 (s, 4H), 6.42 (s, 1H), 6.13 (d, J=8.0 Hz, 1H), 5.57 (s, 1H), 3.85 (d, J=9.0 Hz, 2H), 3.45 (s, 4H), 3.25 (d, J=11.5 Hz, 4H), 3.12 (d, J=27.0 Hz, 1H), 2.49-2.39 (m, 2H), 2.15-1.75 (m, 7H), 1.74-1.50 (m, 7H), 1.45 (s, 1H), 1.25 (d, J=13.0 Hz, 4H).

### Compound 98

¹H NMR (400 MHz, DMSO): δ 11.69 (s, 1H), 8.55 (m, 2H), 8.04 (d, J=2 Hz, 1H), 7.81 (s, 1H), 7.49-7.55 (m, 3H), 7.04-7.10 (m, 5H), 6.4 (s, 1H), 6.2 (m, 1H), 5.61 (s, 1H), 3.82-3.86 (m, 2H), 3.62-3.67 (m, 4H), 3.23-3.26 (m, 4H), 2.21 (m, 2H), 1.98-2.05 (m, 8H), 1.4-1.6 (m, 6H), 1.2 (m 4H).

### Compound 99

¹H NMR (400 MHz, DMSO) δ 11.67 (s, 1H), 11.51 (s, 1H), 8.69-8.42 (m, 2H), 8.04 (d, J=2.5 Hz, 1H), 7.79 (d, J=7.5 Hz, 1H), 7.50 (dd, J=9.5, 6.1 Hz, 3H), 7.26 (dt, J=22.6, 11.3 Hz, 1H), 7.06 (dd, J=11.6, 8.3 Hz, 3H), 6.70 (d, J=9.0 Hz, 1H), 6.47-6.32 (m, 1H), 6.23 (s, 1H), 3.60-3.39 (m, 8H), 3.26-2.97 (m, 7H), 2.60 (dd, J=16.2, 8.2 Hz, 1H), 2.20-1.80 (m, 8H), 1.71-1.37 (m, 8H).

### Compound 100

¹H NMR (400 MHz, DMSO): δ 11.65 (s, 1H), 8.46-8.52 (m, 2H), 8.02 (s, 1H), 7.74 (d, J=9.2 Hz, 1H), 7.53-7.48 (m, 3H), 7.28 (d, J=6.8 Hz, 1H), 7.08-7.00 (m, 3H), 6.7 (d, J=8.4 Hz, 1H), 6.37 (s, 1H), 6.24 (s, 1H), 3.16-3.03 (m, 14H), 2.09-2.07 (m, 3H), 1.90-1.87 (m, 4H), 1.57 (m, 4H), 1.23 (m, 12H), 1.20-1.16 (m, 3H).

### Compound 101

¹H NMR (400 MHz, DMSO) δ 11.62 (s, 1H), 8.51 (s, 2H), 8.00 (s, 1H), 7.79 (s, 1H), 7.60-7.39 (m, 3H), 7.28 (d, J=6.7 Hz, 1H), 7.08 (d, J=7.3 Hz, 2H), 6.97 (s, 1H), 6.69 (d, J=8.5 Hz, 1H), 6.35 (s, 1H), 6.24 (s, 2H), 3.85 (d, J=11.4 Hz, 1H), 3.31-3.19 (m, 5H), 3.11 (s, 6H), 2.99 (d, J=11.3 Hz, 1H), 2.70-2.60 (m, 1H), 2.11 (s, 3H), 1.95-1.85 (m, 1H), 1.60 (s, 2H), 1.46 (s, 3H), 1.30-1.10 (m, 2H), 1.01-0.90 (m, 7H).

### Compound 102

¹H NMR (400 MHz, CDCl₃) δ 10.15 (s, 1H), 9.19 (s, 1H), 8.90 (d, J=2.2 Hz, 1H), 8.52 (d, J=5.1 Hz, 1H), 8.26-8.12 (m, 2H), 7.97 (d, J=9.1 Hz, 1H), 7.71 (d, J=2.4 Hz, 1H), 7.48-7.35 (m, 1H), 7.25-7.17 (m, 1H), 7.01-6.85 (m, 3H), 6.67-6.38 (m, 2H), 5.98 (d, J=2.1 Hz, 1H), 3.90-3.61 (m, 2H), 3.35 (t, J=13.1 Hz, 1H), 3.26-2.98 (m, 8H), 2.48 (s, 2H), 2.27-1.82 (m, 7H), 1.72-1.68 (m, 2H), 1.41-1.10 (m, 9H), 0.87 (d, J=7.0 Hz, 1H).

### Compound 103

¹H NMR (400 MHz, DMSO) δ 11.70-11.50 (m, 2H), 8.56 (s, 2H), 8.04 (s, 1H), 7.80 (s, 1H), 7.51 (d, J=9.0 Hz, 3H), 7.28 (dd, J=7.2, 2.0 Hz, 1H), 7.08 (d, J=7.0 Hz, 3H), 6.71 (d, J=8.6 Hz, 1H), 6.38 (s, 1H), 6.23 (s, 1H), 4.02 (s, 1H), 3.39 (s, 2H), 3.22 (s, 1H), 3.14 (s, 3H), 3.06 (s, 2H), 2.36 (d, J=22.6 Hz, 1H), 2.09 (s, 3H), 2.04-1.91 (m, 1H), 1.86 (s, 2H), 1.60 (s, 1H), 1.48 (d, J=15.9 Hz, 2H), 1.44-1.32 (m, 9H), 1.22-1.19 (m, 8H), 0.84 (d, J=7.0 Hz, 1H).

### Compound 104

¹H NMR (500 MHz, DMSO) δ 11.49 (s, 1H), 8.36 (d, J=2.0 Hz, 1H), 8.23 (s, 1H), 7.91 (d, J=2.5 Hz, 1H), 7.58 (dd, J=19.0, 9.0 Hz, 2H), 7.40 (s, 1H), 7.26 (dd, J=21.5, 5.0 Hz, 2H), 7.15-7.02 (m, 2H), 6.71-6.68 (m, 2H), 6.30 (s, 2H), 3.46 (s, 2H), 3.24 (d, J=6.0 Hz, 2H), 3.08 (s, 5H), 2.94 (d, J=35.0 Hz, 4H), 2.57 (dd, J=18.0, 10.0 Hz, 1H), 2.13 (d, J=5.5 Hz, 3H), 1.96 (dd, J=21.5, 11.5 Hz, 3H), 1.85 (s, 1H), 1.68 (d, J=28.0 Hz, 2H), 1.62-1.58 (m, 5H), 1.50 (t, J=13.0 Hz, 1H), 1.21-1.18 (m, 3H).

### Compound 105

¹H NMR (500 MHz, DMSO) δ 11.51 (s, 1H), 8.41 (s, 1H), 8.26 (s, 2H), 7.92 (s, 1H), 7.68 (d, J=7.5 Hz, 1H), 7.59 (d, J=9.0 Hz, 1H), 7.41 (s, 1H), 7.25 (t, J=19.0 Hz, 2H), 7.15-7.01 (m, 2H), 6.86 (s, 1H), 6.66 (d, J=7.0 Hz, 1H), 6.29 (d, J=11.5 Hz, 2H), 3.96 (s, 1H), 3.59-3.44 (m, 2H), 3.24 (d, J=6.5 Hz, 1H), 3.18-3.00 (m, 6H), 2.94 (d, J=10.5 Hz, 1H), 2.58 (s, 1H), 2.43 (s, 1H), 2.20-2.05 (m, 2H), 2.03-1.89 (m, 2H), 1.84 (s, 1H), 1.79-1.39 (m, 5H), 1.30 (d, J=11.0 Hz, 2H), 1.18 (s, 2H).

### Compound 106

¹H NMR (400 MHz, DMSO) δ 11.49 (s, 1H), 8.39 (d, J=2.0 Hz, 1H), 8.23 (s, 1H), 7.91 (d, J=2.6 Hz, 1H), 7.64 (dd, J=26.2, 8.9 Hz, 2H), 7.47-7.35 (m, 1H), 7.28 (dd, J=7.5, 1.7 Hz, 2H), 7.17-7.00 (m, 2H), 6.83 (d, J=9.3 Hz, 1H), 6.65 (dd, J=8.8, 2.3 Hz, 1H), 6.29 (d, J=3.5 Hz, 2H), 3.81 (s, 1H), 3.42 (d, J=9.0 Hz, 2H), 3.24 (d, J=5.9 Hz, 1H), 3.08-3.04 (m, 4H), 2.74 (s, 3H), 2.59 (s, 1H), 2.39-3.35 (m, 1H), 2.19-2.06 (m, 2H), 2.02-1.98 (m, 4H), 1.84 (s, 1H), 1.72-1.70 (m, 1H), 1.55-1.52 (m, 3H), 1.23-1.20 (m, 6H), 0.85 (t, J=6.8 Hz, 1H).

### Compound 107

¹H NMR (500 MHz, DMSO) δ 11.61 (s, 1H), 11.50 (s, 1H), 8.48-8.43 (m, 2H), 7.99 (s, 1H), 7.71 (s, 1H), 7.54 (d, J=9.0 Hz, 1H), 7.46 (s, 2H), 7.27 (d, J=7.5 Hz, 1H), 7.18-7.03 (m, 2H), 6.95 (s, 1H), 6.68 (d, J=8.0 Hz, 1H), 6.35 (s, 1H), 6.25 (s, 1H), 3.45-3.37 (m, 3H), 3.23 (d, J=6.0 Hz, 1H), 3.09-3.05 (m, 7H), 2.97 (s, 2H), 2.82 (s, 3H), 2.66-2.56 (m, 1H), 2.11 (s, 3H), 1.95-1.91 (m, 3H), 1.84 (s, 1H), 1.69-1.46 (m, 8H), 1.22-1.18 (m, 2H).

### Compound 108

¹H NMR (400 MHz, DMSO) δ 11.70 (s, 2H), 9.26 (s, 1H), 8.56 (dd, J=9.9, 3.9 Hz, 2H), 8.05 (d, J=2.5 Hz, 1H), 7.90-7.72 (m, 1H), 7.64-7.41 (m, 3H), 7.16-7.12 (m, 3H), 6.75 (s, 1H), 6.50-6.34 (m, 1H), 6.28 (s, 1H), 4.06 (s, 4H), 3.46 (s, 2H), 3.35-3.01 (m, 8H), 2.97-2.77 (m, 2H), 2.75-2.72 (m, 3H), 2.05-2.01 (m, 3H), 1.91-1.88 (m, 3H), 1.78-1.74 (m, 2H), 1.69-1.52 (m, 5H), 1.24 (s, 3H).

### Compound 109

¹H NMR (400 MHz, DMSO) δ 11.55 (s, 1H), 8.42 (s, 1H), 8.31 (s, 1H), 7.95 (d, J=2.5 Hz, 1H), 7.64-7.61 (m, 2H), 7.47-7.37 (m, 1H), 7.37-7.22 (m, 2H), 7.20-7.02 (m, 2H), 6.85 (s, 2H), 6.76-6.55 (m, 1H), 6.42-6.23 (m, 2H), 3.42-3.38 (m, 4H), 3.24 (d, J=5.9 Hz, 1H), 3.15-3.11 (m, 8H), 2.84-2.56 (m, 5H), 2.19-2.02 (m, 4H), 1.94-1.90 (m, 5H), 1.75-1.44 (m, 9H), 1.38 (s, 9H).

### Compound 110

¹H NMR (400 MHz, DMSO) δ 11.72 (d, J=14.8 Hz, 2H), 10.78 (s, 1H), 8.66-8.50 (m, 2H), 8.43 (s, 2H), 8.06 (s, 1H), 7.82 (d, J=9.2 Hz, 1H), 7.67-7.42 (m, 5H), 7.27 (s, 1H), 7.10 (d, J=9.5 Hz, 1H), 6.76 (d, J=8.6 Hz, 1H), 6.40-6.30 (m, 2H), 4.72 (s, 1H), 3.50-3.30 (m, 12H), 3.10-3.00 (m, 6H), 2.70-2.60 (m, 2H), 2.19-1.82 (m, 8H), 1.81-1.54 (m, 6H), 1.40 (s, 2H).

### Compound 111

¹H NMR (400 MHz, DMSO) δ 11.52 (s, 1H), 8.38 (s, 1H), 8.26 (s, 1H), 8.16 (s, 1H), 7.92 (s, 1H), 7.58 (d, J=8.8 Hz, 2H), 7.41 (s, 1H), 7.28 (dd, J=7.5, 1.7 Hz, 2H), 7.20-6.94 (m, 2H), 6.89-6.53 (m, 2H), 6.29 (s, 2H), 3.25 (s, 4H), 3.09 (s, 6H), 2.59 (d, J=7.4 Hz, 1H), 2.12 (s, 4H), 1.98-1.91 (m, 8H), 1.56 (s, 5H), 1.47 (s, 3H), 1.40 (s, 1H), 1.21-1.18 (m, 3H).

### Compound 112

¹H NMR (400 MHz, DMSO) δ 11.54 (s, 1H), 8.42 (d, J=1.9 Hz, 1H), 8.30 (s, 1H), 7.94 (t, J=6.3 Hz, 2H), 7.64 (d, J=9.0 Hz, 1H), 7.57 (d, J=8.7 Hz, 1H), 7.47-7.39 (m, 1H), 7.33 (s, 1H), 7.28 (dd, J=7.4, 1.9 Hz, 1H), 7.16-7.03 (m, 2H), 6.82 (d, J=9.1 Hz, 1H), 6.67 (dd, J=8.9, 2.2 Hz, 1H), 6.40-6.19 (m, 2H), 3.24 (d, J=5.3 Hz, 5H), 3.17-2.98 (m, 6H), 2.64 (d, J=23.5 Hz, 4H), 2.57 (dd, J=14.7, 6.6 Hz, 2H), 2.18-2.03 (m, 3H), 2.03-1.85 (m, 4H), 1.81 (s, 3H), 1.67 (s, 2H), 1.63-1.42 (m, 7H), 1.21-1.18 (m, 3H).

### Compound 113

¹H NMR (400 MHz, DMSO) δ 11.59 (s, 1H), 8.47 (d, J=1.8 Hz, 1H), 8.36 (s, 1H), 7.99 (d, J=2.5 Hz, 1H), 7.69 (d, J=8.5 Hz, 1H), 7.55 (d, J=8.8 Hz, 1H), 7.44 (dd, J=12.2, 9.4 Hz, 2H), 7.28 (dd, J=7.2, 2.0 Hz, 1H), 7.08 (q, J=7.4 Hz, 2H), 6.90 (d, J=8.9 Hz, 1H), 6.68 (dd, J=8.9, 2.0 Hz, 1H), 6.38-6.30 (m, 1H), 6.25 (d, J=8.5 Hz, 1H), 6.07 (ddd, J=55.8, 30.1, 4.2 Hz, 1H), 3.27-3.18 (m, 2H), 3.17-2.97 (m, 6H), 2.71-2.56 (m, 4H), 2.43 (s, 2H), 2.36 (s, 2H), 2.18-1.93 (m, 5H), 1.92-1.77 (m, 3H), 1.63-1.39 (m, 8H), 1.32-1.12 (m, 2H).

### Compound 114

¹H NMR (400 MHz, DMSO) δ 11.58 (s, 1H), 8.45 (d, J=2.0 Hz, 1H), 8.35 (s, 1H), 7.97 (d, J=2.5 Hz, 1H), 7.68 (d, J=8.8 Hz, 1H), 7.55 (d, J=8.8 Hz, 1H), 7.46-7.40 (m, 1H), 7.38 (d, J=2.3 Hz, 1H), 7.28 (dd, J=7.3, 1.9 Hz, 1H), 7.08 (q, J=7.7 Hz, 2H), 6.88 (d, J=9.3 Hz, 1H), 6.67 (d, J=8.8 Hz, 1H), 6.38-6.16 (m, 2H), 4.65 (t, J=4.8 Hz, 1H), 4.53 (t, J=4.7 Hz, 1H), 3.43-3.38 (m, 3H), 3.26-3.17 (m, 2H), 3.16-2.99 (m, 6H), 2.85-2.81 (m, 2H), 2.66-2.51 (m, 2H), 2.23-1.81 (m, 8H), 1.58-1.52 (m, 9H), 1.27-1.08 (m, 2H).

### Compound 115

¹H NMR (400 MHz, DMSO) δ 11.66 (s, 1H), 11.54 (s, 1H), 8.53 (s, 1H), 8.47 (s, 1H), 8.03 (d, J=2.4 Hz, 1H), 7.76 (d, J=9.3 Hz, 1H), 7.50 (dd, J=13.2, 6.0 Hz, 3H), 7.28 (dd, J=7.0, 2.2 Hz, 1H), 7.08 (d, J=7.2 Hz, 2H), 7.01 (d, J=8.9 Hz, 1H), 6.70 (d, J=8.9 Hz, 1H), 6.37 (s, 1H), 6.23 (s, 1H), 3.45-3.38 (m, 3H), 3.22 (d, J=6.2 Hz, 1H), 3.19-2.98 (m, 8H), 2.63-2.55 (m, 1H), 2.45 (s, 3H), 2.09 (d, J=5.6 Hz, 3H), 2.00-1.76 (m, 4H), 1.66-1.40 (m, 9H), 1.26-1.08 (m, 2H).

### Compound 116

¹H NMR (400 MHz, DMSO) δ 12.05 (s, 1H), 8.68-8.55 (m, 2H), 7.93 (dd, J=9.1, 2.1 Hz, 1H), 7.73 (d, J=8.7 Hz, 2H), 7.28 (dd, J=27.2, 8.3 Hz, 2H), 7.12 (d, J=9.6 Hz, 2H), 6.92 (d, J=8.7 Hz, 2H), 3.52-3.43 (m, 3H), 3.23-3.18 (m, 6H), 3.11 (s, 1H), 2.63-2.61 (m, 2H), 2.17 (s, 3H), 1.96-1.91 (m, 4H), 1.69-1.44 (m, 7H), 1.44-1.33 (m, 12H), 1.25-1.21 (m, 3H).

### Compound 117

¹H NMR (400 MHz, DMSO) δ 12.08 (s, 1H), 8.62 (d, J=12.1 Hz, 2H), 8.31 (s, 2H), 7.94 (d, J=9.0 Hz, 1H), 7.76 (s, 2H), 7.21 (m, 3H), 6.95 (s, 2H), 4.01 (m, 6H), 3.50 (s, 3H), 3.13 (s, 2H), 3.01-2.95 (m, 5H), 2.65-2.60 (m, 3H), 2.15-2.12 (m, 2H), 1.99-1.87 (m, 3H), 1.70-1.64 (m, 8H).

### Compound 118

¹H NMR (400 MHz, DMSO) δ 12.04 (s, 1H), 8.60 (d, J=2.0 Hz, 1H), 8.52 (s, 1H), 7.93 (d, J=7.4 Hz, 1H), 7.73 (d, J=8.8 Hz, 2H), 7.31 (d, J=7.6 Hz, 1H), 7.20 (d, J=9.3 Hz, 1H), 7.17-7.07 (m, 2H), 6.89 (d, J=8.9 Hz, 2H), 3.54-3.42 (m, 3H), 3.32-3.28 (m, 7H), 3.12-3.08 (m, 1H), 2.65-261 (m, 4H), 2.17 (s, 3H), 2.03-1.87 (m, 7H), 1.62-1.58 (m, 5H), 1.48-1.42 (m, 2H), 1.39 (s, 1H), 1.31-1.13 (m, 2H).

### Compound 119

¹H NMR (400 MHz, DMSO) δ 8.51 (s, 1H), 8.38 (s, 1H), 7.89 (d, J=11.3 Hz, 1H), 7.72 (d, J=8.9 Hz, 2H), 7.31 (d, J=7.7 Hz, 1H), 7.20-7.01 (m, 3H), 6.82 (d, J=9.1 Hz, 2H), 3.46 (s, 3H), 3.31-3.06 (m, 8H), 2.68-2.55 (m, 3H), 2.17 (s, 3H), 2.08-1.78 (m, 6H), 1.74-1.50 (m, 7H), 1.37 (d, J=13.0 Hz, 9H), 1.23 (s, 6H).

### Compound 120

¹H NMR (400 MHz, DMSO) δ 12.15 (s, 1H), 9.79 (s, 1H), 8.63 (dd, J=9.8, 3.9 Hz, 2H), 8.13 (s, 2H), 7.95 (dd, J=9.2, 2.0 Hz, 1H), 7.78 (d, J=8.2 Hz, 2H), 7.27 (d, J=9.5 Hz, 2H), 6.97 (s, 2H), 4.51 (s, 6H), 3.47-3.44 (m, 2H), 3.17-3.12 (m, 8H), 2.89 (s, 2H), 2.18-1.50 (m, 18H).

### Compound 121

¹H NMR (400 MHz, DMSO) δ 11.53 (s, 1H), 8.38 (s, 1H), 8.28 (br, s, 1H), 7.93 (d, J=2.5 Hz, 1H), 7.59 (d, J=8.7 Hz, 2H), 7.45-7.36 (m, 1H), 7.28-7.26 (m, 2H), 7.15-7.02 (m, 2H), 6.78 (d, J=9.3 Hz, 1H), 6.67-6.64 (m, 1H), 6.29-6.27 (m, 2H), 5.16 (br, s, 1H), 3.65 (s, 2H), 3.65 (br, s, 2H), 3.43-3.34 (m, 5H), 3.09-3.25 (m, 9H), 2.15-1.47 (m, 8H), 1.23-1.11 (m, 2H).

### Compound 122

¹H NMR (400 MHz, DMSO) δ 11.65 (s, 1H), 8.53 (d, J=1.9 Hz, 1H), 8.46 (br, s, 1H), 8.03 (d, J=2.4 Hz, 1H), 7.77 (d, J=9.3 Hz, 1H), 7.59-7.43 (m, 3H), 7.27 (dd, J=7.1, 2.0 Hz, 1H), 7.15-6.95 (m, 3H), 6.70 (d, J=9.2 Hz, 1H), 6.37 (s, 1H), 6.23 (s, 1H), 4.09 (q, J=7.1 Hz, 2H), 7.41-7.39 (m, 4H), 3.23-3.03 (m, 7H), 2.71-2.52 (m, 3H), 2.21-1.85 (m, 8H), 1.60-1.46 (m, 9H), 1.23-1.17 (m, 6H).

### Compound 123

¹H NMR (400 MHz, DMSO) δ 11.81 (s, 1H), 11.73 (s, 1H), 11.42 (br, 1H), 8.59-8.50 (m, 2H), 8.14-8.06 (s, 1H), 7.59-7.48 (m, 5H), 7.32 (br, s, 1H), 7.09 (d, J=8 Hz, 2H), 6.40 (s, 1H), 6.29 (s, 1H), 4.73 (br, s 1H), 4.07 (s, 2H), 3.32-3.02 (m, 12H), 2.62-2.60 (m, 1H), 2.08-1.62 (m, 11H), 2.37-1.21 (m, 7H).

### Compound 124

¹H NMR (400 MHz, DMSO) δ 11.68 (s, 1H), 11.51 (s, 1H), 8.56-5.51 (s, 2H), 8.05 (s, 1H), 7.80 (d, J=7.2 Hz, 1H), 7.53 (d, J=8.0 Hz, 2H), 7.49 (s, 1H), 7.29-7.27 (m, 1H), 7.10-7.07 (m, 3H), 6.71 (d, J=8.0 Hz, 1H), 6.38 (s, 1H), 6.23 (s, 1H), 3.45-3.38 (m, 4H), 3.23-3.04 (m, 5H), 2.84 (br, s, 1H), 2.62-2.60 (m, 1H), 2.09-1.93 (m, 8H), 1.57-1.44 (m, 8H), 1.41-1.23 (m, 6H), 0.97 (d, J=4, 6H).

### Compound 125

¹H NMR (400 MHz, DMSO) δ 11.70 (s, 1H), 11.67 (br, s, 1H), 8.57 (s, 1H), 5.53 (br, s, 1H), 8.05-7.98 (m, 4H), 7.84-7.79 (m, 1H), 7.63-7.46 (m, 3H), 7.11-7.09 (m, 2H), 6.75 (s, 1H), 6.39 (s, 1H), 6.29 (s, 1H), 4.27 (s, 1H), 3.46-3.10 (m, 15H), 2.66-2.62 (m, 1H), 1.98-1.48 (m, 17H), 0.98 (d, J=4.0 Hz, 3H), 0.89 (d, J=4.0 Hz, 3H).

### Compound 126

¹H NMR (500 MHz, DMSO) δ 11.70 (s, 1H), 11.68 (br, s, 1H), 9.90 (s, 1H), 8.57 (d, J=2.0 Hz, 1H), 8.53-8.51 (m, 1H), 8.06 (d, J=2.0 Hz, 1H), 7.83-7.81 (m, 1H), 7.60-7.52 (m, 3H), 7.43-7.20 (m, 3H), 7.09 (d, J=9.5 Hz, 1H), 6.75 (d, J=9, 1H), 6.40 (s, 1H), 6.29 (s, 1H), 3.83-3.23 (m, 22H), 2.79-1.75 (m, 15H).

### Compound 127

¹H NMR (400 MHz, DMSO) δ 11.68 (s, 1H), 11.53 (s, 1H), 8.54-8.51 (m, 2H), 8.04 (s, 1H), 7.77 (br, s, 1H), 7.52-750 (m, 3H), 7.28 (dd, J=7.0, 2.2 Hz, 1H), 7.14-6.88 (m, 3H), 6.70 (d, J=8.9 Hz, 1H), 6.38 (s, 1H), 6.23 (s, 1H), 3.45-3.04 (m, 14H), 2.61-2.55 (m, 2H), 2.10-2.08 (m, 3H), 1.94-1.65 (m, 4H), 1.65-1.57 (m, 8H), 1.23-1.15 (m, 2H), 0.95-0.89 (m, 4H).

### Compound 128

¹H NMR (400 MHz, DMSO) δ 11.72 (s, 1H), 11.68 (br, s, 1H), 8.57-8.53 (m, 2H), 8.05 (d, J=2.4 Hz, 1H), 7.81 (dd, J=9.2, 2.0 Hz, 1H), 7.56-7.24 (m, 6H), 7.08 (d, J=9.4 Hz, 1H), 6.75 (d, J=8.4 Hz, 1H), 6.40-6.38 (m, 1H), 6.29 (s, 1H), 4.65-4.48 (m, 2H), 3.46-3.43 (m, 2H), 3.26-3.10 (m, 12H), 2.54-2.50 (m, 2H), 1.92-1.52 (m, 6H), 1.50-1.18 (m, 15H).

### Compound 129

¹H NMR (500 MHz, DMSO) δ 11.51 (s, 1H), 8.39 (s, 1H), 8.28 (s, 1H), 7.93 (d, J=2.4 Hz, 1H), 7.60-7.58 (m, 2H), 7.41 (br, s, 1H), 7.32-7.24 (m, 2H), 7.15-7.04 (m, 2H), 6.80 (br, s, 1H), 6.65 (d, J=8.8 Hz, 1H), 6.29 (d, J=7.4 Hz, 2H), 3.44-3.30 (m, 3H), 3.07-3.05 (m, 5H), 2.82-2.58 (m, 4H), 2.15-1.42 (m, 16H), 1.24-1.17 (m, 2H), 00.99 (br, s 1H), 0.59 (d, J=8.0 Hz, 2H), 0.28 (s, 2H).

### Compound 130

¹H NMR (400 MHz, DMSO) δ 11.58 (s, 1H), 8.45 (d, J=2.0 Hz, 1H), 8.36 (br, s, 1H), 7.97 (d, J=2.8 Hz, 1H), 7.67 (d, J=8.6 Hz, 1H), 7.56 (d, J=8.8 Hz, 1H), 7.49-7.43 (m, 1H), 7.37 (s, 1H), 7.28 (dd, J=7.3, 1.9 Hz, 1H), 7.10-7.05 (m, 2H), 6.87 (d, J=9.3 Hz, 1H), 6.68 (d, J=7.1 Hz, 1H), 6.33 (s, 1H), 6.26 (d, J=1.8 Hz, 1H), 4.08 (q, J=7.1 Hz, 2H), 3.35-3.10 (m, 11H), 2.64-2.61 (4H), 2.11-1.90 (m, 8H), 1.59-1.52 (m, 10H), 1.23-0.85 (m, 5H).

### Compound 131

¹H NMR (400 MHz, DMSO) δ 11.72 (s, 1H), 11.69 (br, s, 1H), 9.33 (s, 1H), 8.57-8.55 (m, 2H), 8.07 (s, 1H), 7.83 (d, J=9.1 Hz, 1H), 7.62-7.47 (m, 3H), 7.45 (br, s, H), 7.30-7.23 (m 1H), 7.10 (d, J=9.3 Hz, 1H), 6.76 (d, J=7.2 Hz, 1H), 6.40 (s, 1H), 6.30 (s, 1H), 3.46-3.05 (m, 13H), 3.10-3.05 (m, 5H), 2.04-1.61 (m, 13H), 1.50-1.49 (m, 2H), 1.22-1.18 (m, 3H).

### Compound 132

¹H NMR (400 MHz, DMSO) δ 11.70 (s, 1H), 11.52 (s, 1H), 8.57 (d, J=2.4 Hz, 1H), 8.51-8.48 (m, 1H), 8.05 (d, J=2.4, 1H), 7.81 (dd, J=9.2, 2.0 Hz, 1H), 7.60-7.45 (m, 3H), 7.27 (dd, J=6.6, 2.5 Hz, 1H), 7.08-7.05 (m 3H), 6.70 (d, J=7.2 Hz, 1H), 6.39-6.38 (m, 1H), 6.22 (d, J=1.6 Hz, 1H), 3.42-3.91 (m, 3H), 3.21-3.02 (m, 6H), 2.67-2.50 (m, 2H), 2.01-1.81 (m, 4H), 1.59-1.16 (16H).

### Compound 133

¹H NMR (400 MHz, DMSO) δ 11.68 (s, 1H), 11.60 (br, s, 1H), 8.55 (d, J=2.1 Hz, 1H), 8.49 (t, J=5.2 Hz, 1H), 8.05 (d, J=2.5 Hz, 1H), 7.79 (dd, J=9.2, 1.7 Hz, 1H), 7.58-7.44 (m, 3H), 7.27 (dd, J=6.6, 2.6 Hz, 1H), 6.70 (d, J=7.4 Hz, 1H), 6.38 (dd, J=3.0, 1.8 Hz, 1H), 6.23 (s, 1H), 3.82 (s, 4H), 3.43-3.41 (m, 3H), 3.32-3.02 (m, 5H), 2.10-2.07 (m, 3H), 1.91-1.86 (m, 4H), 1.51-1.43 (m, 13H), 1.01-0.97 (m, 2H).

### Compound 134

¹H NMR (400 MHz, DMSO) δ 11.65 (s, 1H), 11.55 (br, s, 1H), 8.52 (s, 1H), 8.43 (s, 1H), 8.02 (d, J=2.3 Hz, 1H), 7.74 (d, J=8.7 Hz, 1H), 7.58-7.39 (m, 3H), 7.28 (dd, J=7.1, 2.2 Hz, 1H), 7.18-7.01 (m, 2H), 6.98 (d, J=8.5 Hz, 1H), 6.70 (dd, J=9.0, 1.9 Hz, 1H), 6.36 (d, J=1.2 Hz, 1H), 6.24 (d, J=1.7 Hz, 1H), 4.40 (d, J=4.0 Hz, 1H), 3.36 (s, 55H), 3.23 (d, J=5.8 Hz, 1H), 3.12-3.03 (m, 7H), 2.57-2.50 (m, 5H), 2.10-1.46 (m, 14H), 1.23-1.13 (m, 6H).

### Compound 135

¹H NMR (400 MHz, DMSO) δ 11.81 (s, 1H), 8.66 (br, s, 1H), 8.62 (m, s, 1H), 8.30 (s, 1H), 8.18 (d, J=2.3 Hz, 1H), 7.91 (d, J=9.2 Hz, 1H), 7.69-7.58 (m, 3H), 7.43 (dd, J=7.0, 2.2 Hz, 1H), 7.39-7.05 (m, 3H), 6.85 (d, J=8.8 Hz, 1H), 6.52 (s, 1H), 6.39 (s, 1H), 3.59-3.51 (m, 4H), 3.39-3.18 (m, 6H), 2.77-2.56 (m, 4H), 2.25-1.61 (m, 15H), 1.39-1.32 (m, 2H).

### Compound 136

¹H NMR (400 MHz, DMSO) δ 11.68 (s, 1H), 11.53 (s, 1H), 8.54 (br, s, 1H), 8.47 (br, s, 1H), 8.04 (d, J=2.4 Hz, 1H), 7.77 (d, J=9.0 Hz, 1H), 7.52-7.49 (m, 3H), 7.28 (dd, J=7.0, 2.3 Hz, 1H), 7.10-7.01 (m, 3H), 6.70 (d, J=8.9 Hz, 1H), 6.38 (s, 1H), 6.23 (s, 1H), 3.41-3.39 (m, 2H), 3.28-3.03 (m, 6H), 2.57-2.50 (m, 2H), 2.10-1.82 (m, 7H), 1.60-1.39 (m, 9H), 1.19-1.10 (m, 6H), 0.84 (s, 6H).

### Example 42: Biological Assays

The HTRF BCL-2/BAK or BCL-XL/BAK assay from Cisbio (63ADK000CB01PEG; 63ADK000CB04PEG) was used to test compounds disclosed herein for blocking of BCL-2 or BCL-XL protein with its ligand, BAK. Recombinant human 2 nM Tag1-BCL-2, 2 nM Tag1-BCL-XL protein, 10 nM Tag2-BAK/5 nM Tag2-BAK (corresponding to BCL-2 and BCL-XL assay, respectively) were pre-incubated with a serial dilution of compounds disclosed herein (maximum concentration and dilution ratio determined by the results of pre-experiment may vary) at room temperature for 15 minutes in an assay buffer from BCL-2/BAK or BCL-XL/BAK assay kits, respectively. Then the pre-mixed anti-Tag1-Eu³⁺ and anti-tag2-XL665 were added to the plate and further incubated at room temperature for another 2 hours. The signals (665 nM, 615 nM) were read on Envision 2104 instrument. The IC₅₀ for each compound was derived from fitting the signal of 665/615 nM to the increasing compound concentration.

Using the above assays, the following compounds were tested. In Table 2A and Table 2B below, for IC₅₀ data, “***” means the compound had an IC₅₀ of greater than zero but less than or equal to about 20 nM; “**” means the compound had an IC₅₀ of greater than about 20 nM but less than or equal to about 200 nM; “*” means the compound had an IC₅₀ of greater than about 200 nM but less than or equal to about 2000 nM; “-” means the compound had an IC₅₀ of greater than about 2000 nM.

TABLE 2A
BCL-2 IC₅₀ data of Exemplary Compounds

| Compound No. | IC₅₀ BCL-2 (nM) |
|---|---|
| Venetoclax | *** |
| 1 | * |
| 2 | ** |
| 3 | * |
| 4 | ** |
| 5 | ** |
| 6 | *** |
| 8 | * |
| 9 | * |
| 10 | * |
| 11 | * |
| 12 | ** |
| 13 | ** |
| 14 | * |
| 15 | * |
| 16 | *** |
| 17 | ** |
| 18 | *** |
| 19 | *** |
| 20 | *** |
| 21 | *** |
| 22 | ** |
| 23 | ** |
| 25 | ** |
| 26 | *** |
| 27 | *** |
| 28 | ** |
| 29 | *** |
| 30 | *** |
| 31 | *** |
| 32 | *** |
| 33 | *** |
| 34 | *** |
| 35 | ** |
| 36 | *** |
| 37 | *** |
| 38 | *** |
| 39 | *** |
| 40 | ** |
| 41 | *** |
| 42 | *** |
| 43 | *** |
| 44 | *** |
| 45 | *** |
| 46 | *** |
| 47 | ** |
| 49 | *** |
| 50 | *** |
| 51 | *** |
| 52 | ** |
| 53 | *** |
| 54 | *** |
| 55 | ** |
| 56 | ** |
| 57 | *** |
| 58 | *** |
| 59 | *** |
| 60 | *** |
| 61 | *** |
| 62 | ** |
| 63 | ** |
| 64 | *** |
| 65 | *** |
| 66 | *** |
| 67 | *** |
| 68 | *** |
| 69 | *** |
| 70 | *** |
| 71 | *** |
| 72 | *** |
| 73 | *** |
| 74 | *** |
| 75 | *** |
| 76 | *** |
| 77 | *** |
| 78 | *** |
| 79 | *** |
| 80 | *** |
| 81 | ** |
| 82 | *** |
| 83 | *** |
| 84 | *** |
| 85 | *** |
| 86 | *** |
| 87 | *** |
| 88 | *** |
| 91 | *** |
| 92 | ** |
| 93 | ** |
| 94 | *** |
| 95 | ** |
| 96 | ** |
| 97 | ** |
| 98 | ** |
| 99 | *** |
| 100 | *** |
| 101 | *** |
| 102 | *** |
| 103 | *** |
| 104 | *** |
| 105 | *** |
| 107 | *** |
| 108 | *** |
| 109 | *** |
| 110 | *** |
| 111 | *** |
| 112 | *** |
| 113 | *** |
| 114 | *** |
| 115 | *** |
| 116 | — |
| 117 | ** |
| 118 | * |
| 119 | ** |
| 120 | ** |
| 121 | *** |
| 122 | *** |
| 123 | *** |
| 124 | *** |
| 125 | *** |
| 126 | *** |
| 127 | *** |
| 128 | *** |
| 129 | *** |
| 130 | *** |
| 131 | *** |
| 132 | *** |
| 133 | *** |
| 134 | *** |
| 135 | *** |

TABLE 2B
BCL-XL IC₅₀ data of Exemplary Compounds

| Compound No. | IC₅₀ BCL-XL (nM) |
|---|---|
| Venetoclax | ** |
| 2 | — |
| 4 | — |
| 5 | * |
| 6 | * |
| 12 | — |
| 13 | * |
| 16 | * |
| 17 | — |
| 18 | ** |
| 19 | — |
| 20 | * |
| 21 | — |
| 22 | — |
| 23 | * |
| 25 | * |
| 26 | — |
| 27 | ** |
| 28 | — |
| 29 | *** |
| 30 | * |
| 31 | ** |
| 32 | *** |
| 33 | ** |
| 34 | * |
| 35 | * |
| 36 | * |
| 37 | * |
| 38 | * |
| 39 | ** |
| 40 | — |
| 41 | *** |
| 42 | *** |
| 43 | *** |
| 44 | *** |
| 45 | * |
| 46 | ** |
| 47 | — |
| 49 | * |
| 50 | * |
| 51 | * |
| 52 | — |
| 53 | *** |
| 54 | — |
| 56 | — |
| 61 | * |
| 62 | — |
| 63 | — |
| 64 | ** |
| 65 | *** |
| 66 | ** |
| 67 | ** |
| 68 | *** |
| 69 | *** |
| 70 | ** |
| 71 | ** |
| 72 | ** |
| 73 | ** |
| 74 | ** |
| 75 | ** |
| 76 | * |
| 77 | ** |
| 78 | * |
| 79 | * |
| 80 | ** |
| 81 | * |
| 82 | * |
| 83 | *** |
| 84 | ** |
| 85 | *** |
| 86 | *** |
| 87 | ** |
| 88 | *** |
| 99 | ** |
| 100 | * |
| 101 | ** |
| 102 | ** |
| 104 | *** |
| 107 | ** |
| 108 | *** |
| 109 | ** |
| 110 | *** |
| 111 | ** |
| 112 | *** |
| 113 | ** |
| 114 | *** |
| 115 | * |
| 116 | — |
| 117 | ** |
| 118 | — |
| 119 | * |
| 120 | ** |
| 121 | *** |
| 122 | ** |
| 123 | *** |
| 124 | ** |
| 125 | *** |
| 129 | *** |
| 130 | *** |
| 131 | *** |

### Example 43: CYP2C9 Activity Assay

In this assay, several exemplary compounds of the present disclosure and a control compound (Venetoclax) were used as the test compounds. In general, the assay was conducted by the following steps:
1. The solution of test compound, human liver microsomes solution, and substrate (diclofenac) solution were mixed in a 96-well assay plate on ice to, and the final concentration of each test compound is 1 μM;
2. The 96-well assay plate and NADPH solution were pre-incubated at 37° C. for 5 minutes;
3. NADPH solution was added to into the assay plates to initiate the reaction;
4. The assay plate was incubated at 37° C. for 10 min;
5. The reaction was stopped; and after quenching, a portion of the supernatant was taken from each well for LC/MS analysis;
6. The inhibition rate was calculated by the following equation: Inhibition rate (%)=(1−Valueₜₑₛₜ/Value_{control})×100%, where Valueₜₑₛₜ refers to the LC/MS data obtained from wells with test compounds and Value_{control} refers to the LC/MS data obtained from wells without test compound.

The inhibition rates of several test compounds were shown in Table 3 below.

TABLE 3
Inhibition Rate (%) of Exemplary
Compounds against CYP2C9 Enzyme

| Compound No. | Inhibition rate (%) |
|---|---|
| Venetoclax | 49 |
| 94 | <30 |
| 100 | <30 |
| 104 | <30 |
| 107 | 31 |
| 108 | <30 |
| 112 | 33 |
| 113 | <30 |
| 114 | <30 |
| 115 | <30 |

As demonstrated in table above, the compounds of the present disclosure showed a significantly decreased inhibition rate against the reference compound Venetoclax.

### Example 44: Efficacy Study

### Cell Proliferation Assay

The CellTiter-Glo® Luminescent Cell Viability Assay (Promega, G7573) was used to study the cellular potency of disclosed compounds herein. The cells were harvested during the logarithmic growth period and counted with hemocytometer. The DOHH2 cells were seeded at 1.6*10⁴ in 90 ul DMEM medium supplemented with 10% fetal bovine serum (FBS) (as RS4;11 cells were seeded at 4000 in 90 ul RPMI-1640 medium with 10% FBS) per well in 96-well plates and treated with 10 ul of a serial dilution of compounds disclosed herein (maximum concentration and dilution ratio determined by the results of pre-experiment may vary) for 72 hours in a 5% CO₂ incubator at 37° C. Cell viability was assessed according to the manufacturer's recommendations. After the plates return to the room temperature, 100 ul of CellTiter-Glo® reagent was added to 100 ul of cell culture. The mixture was agitated on an orbital shaker for 2 minutes or placed it at room temperature for 10 minutes to allow cell lysis and stabilization of luminescent signals. Luminescent signals were recorded using Envision 2104 instrument. And GI₅₀ values were then calculated.

The GI₅₀ values of the tested compounds are shown in Table 4 below. In Table 4 below, for GI₅₀ data, “***” means the compound had an GI₅₀ of greater than zero but less than or equal to about 50 nM; “**” means the compound had an GI₅₀ of greater than about 50 nM but less than or equal to about 500 nM; “*” means the compound had an GI₅₀ of greater than about 500 nM but less than or equal to about 5000 nM; “-” means the compound had an GI₅₀ of greater than about 5000 nM.

TABLE 4
GI₅₀ Data of Exemplary Compounds

| Compound No. | GI₅₀ DOHH2 (nM) |
|---|---|
| 2 | — |
| 4 | * |
| 5 | * |
| 6 | ** |
| 12 | * |
| 13 | * |
| 16 | * |
| 17 | * |
| 18 | *** |
| 19 | * |
| 20 | ** |
| 21 | ** |
| 22 | * |
| 23 | * |
| 25 | ** |
| 26 | ** |
| 27 | *** |
| 28 | — |
| 29 | *** |
| 30 | * |
| 31 | *** |
| 32 | *** |
| 33 | *** |
| 34 | * |
| 35 | * |
| 36 | ** |
| 37 | ** |
| 38 | *** |
| 39 | *** |
| 40 | * |
| 41 | ** |
| 42 | ** |
| 43 | ** |
| 44 | ** |
| 45 | * |
| 46 | *** |
| 47 | — |
| 49 | * |
| 50 | ** |
| 51 | * |
| 52 | — |
| 53 | ** |
| 54 | * |
| 55 | — |
| 56 | — |
| 57 | * |
| 58 | * |
| 59 | — |
| 60 | — |
| 61 | * |
| 62 | — |
| 63 | — |
| 64 | ** |
| 65 | *** |
| 66 | *** |
| 67 | *** |
| 68 | *** |
| 69 | *** |
| 70 | *** |
| 71 | *** |
| 72 | *** |
| 73 | ** |
| 74 | *** |
| 75 | *** |
| 76 | * |
| 77 | *** |
| 78 | ** |
| 79 | ** |
| 80 | * |
| 81 | * |
| 82 | * |
| 83 | *** |
| 84 | *** |
| 85 | *** |
| 86 | *** |
| 87 | *** |
| 88 | *** |
| 91 | * |
| 92 | * |
| 93 | * |
| 94 | * |
| 95 | * |
| 96 | — |
| 97 | * |
| 98 | * |
| 99 | *** |
| 100 | ** |
| 101 | *** |
| 102 | *** |
| 103 | ** |
| 104 | *** |
| 105 | ** |
| 107 | *** |
| 108 | *** |
| 109 | *** |
| 110 | *** |
| 111 | *** |
| 112 | *** |
| 113 | *** |
| 114 | *** |
| 115 | ** |
| 117 | — |
| 119 | ** |
| 120 | * |
| 121 | *** |
| 122 | *** |
| 123 | ** |
| 124 | *** |
| 125 | *** |
| 126 | *** |
| 127 | *** |
| 128 | *** |
| 129 | *** |
| 130 | *** |
| 131 | *** |
| 132 | ** |
| 133 | *** |
| 134 | *** |
| 135 | *** |
| 136 | — |

### In Vivo Pharmacodynamic Study

In general, NOD/SCID RS4;11 subcutaneous xenograft tumor model was established by inoculating 5*10⁶/0.1 ml/mouse subcutaneously in the right back of the NOD/SCID female mouse. The animals were checked daily for any effects of treatments on behaviors such as mobility, food and water consumption, body weight gain/loss, eyes, hairs and any other abnormalities. Mortality and clinical signs observed during the study were recorded in the raw data. Animal weight and tumor size were measured every two days during the study. Tumor volume (TV) was calculated as: TV=0.5*a*b², wherein a and b represent the measured length and width of tumor, respectively. Relative tumor proliferation inhibition rate (TGI_{RTV} (%)), as an indication of anti-tumor effectiveness, was calculated as: TGI_{RTV} (%)=(1−T_{RTV}/C_{RTV})*100%, wherein T_{RTV} and C_{RTV} were relative tumor volume (RTV) in treatment group and vehicle control group, respectively. RTV was calculated as: RTV=Vₜ/V₀, wherein Vₜ and V₀ represented the tumor volume measured on day t after dosing and on the first day of dosing. At the end of last dosing, plasma and tumor tissue were collected, weighed, and photographed according to requirements of the study protocol.

Compared to the vehicle group, the drug treatment groups showed anti-tumor proliferation effect. The TGI_{RTV}(%) values after 20 days dosing were shown in Table 5 below, where “+++” represented TGI_{RTV}(%)≥800; “++” represented 30%<TGI_{RTV}(%)<80%. p.o. stands for “per os”; I.P. stands for “intraperitoneal”; q.d. refers to “once daily”.

TABLE 5
TGI_{RTV} of Exemplary Compounds

| Compound No. | Drug administration | TGI_{RTV}(%) |
|---|---|---|
| 31 | 25 mg/kg; p.o.; q.d. | ++ |
| 70 | 60 mg/kg; p.o.; q.d. | +++ |
| 104 | 21 mg/kg; I.P.; q.d. | +++ |
| 113 | 50 mg/kg; p.o.; q.d. | +++ |
| 114 | 50 mg/kg; p.o.; q.d. | +++ |

As shown in Table 5 above, the tested compounds of the present disclosure showed potent inhibition of tumor growth.

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents maybe considered to fall within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. A compound of Formula I, 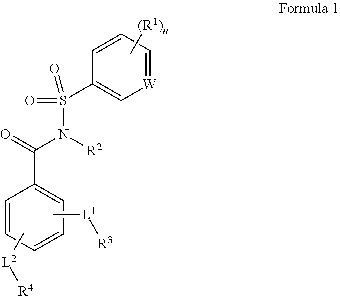
or a tautomer, a stereoisomer, or a pharmaceutically acceptable salt thereof, wherein
W is N or C(R¹);
n is 0, 1, 2 or 3;
each R¹ is independently selected from the group consisting of hydrogen, halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, and —NH-L³-R^{a}, wherein,
L³ is absent or selected from alkyl, alkenyl, or alkynyl, each of which is optionally substituted with one or more R^{b};
R^{a} is selected from the group consisting of cycloalkyl, heterocyclyl, aryl and heteroaryl, wherein each of the cycloalkyl, heterocyclyl, aryl and heteroaryl is optionally substituted with one or more R^{c};
R² is selected from the group consisting of hydrogen, alkyl, alkenyl, alkynyl, heteroalkyl, heteroalkenyl, heteroalkynyl, haloalkyl, and alkylalkoxyl;
L¹ is absent, O, S, or N;
R³ is absent, cycloalkyl, heterocyclyl, aryl or heteroaryl, wherein each of cycloalkyl, heterocyclyl, aryl or heteroaryl is optionally substituted with one or more R^{d};
L² is selected from the group consisting of C₁₋₆ alkyl, C₁₋₆ alkenyl, C₁₋₆ alkynyl, halo-C₁₋₆ alkyl, hetero-C₁₋₆ alkenyl, hetero-C₁₋₆ alkynyl, cycloalkyl, heterocyclyl, aryl and heteroaryl, each of which is optionally substituted with one or more R^{e};
R⁴ is 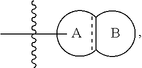
wherein
Ring A is selected from the group consisting of cycloalkyl, heterocyclyl, aryl, and heteroaryl, each of which is optionally substituted with one or more R^{f};
Ring B is selected from the group consisting of cycloalkyl, heterocyclyl, aryl, and heteroaryl, each of which is optionally substituted with one or more R^{g}; [symbol]
is a bond via which Ring A is fused to Ring B;
each R^{c} is independently selected from the group consisting of halogen, cyano, hydroxyl, sulfhydryl, —NH₂, —NO₂, alkyl, alkenyl, alkynyl, heteroalkyl, heteroalkenyl, heteroalkynyl, haloalkyl, alkoxyl, haloalkoxyl, cycloalkyl, heterocyclyl, aryl, heteroaryl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3};
R^{b}, R^{d} and R^{e} are each independently selected from the group consisting of halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, haloalkoxyl, cycloalkyl, heterocyclyl, aryl and heteroaryl;
each R^{f} is independently selected from the group consisting of oxo, halogen, cyano, hydroxyl, sulfhydryl, —NH₂, —NO₂, alkyl, alkenyl, alkynyl, heteroalkyl, heteroalkenyl, heteroalkynyl, haloalkyl, alkoxyl, haloalkoxyl, cycloalkyl, heterocyclyl, aryl, heteroaryl, and —S(O)₂—R^{a4};
each R^{g} is independently selected from the group consisting of oxo, halogen, cyano, hydroxyl, sulfhydryl, —NH₂, —NO₂, alkyl, alkenyl, alkynyl, heteroalkyl, heteroalkenyl, heteroalkynyl, haloalkyl, alkoxyl, haloalkoxyl, cycloalkyl, heterocyclyl, aryl, heteroaryl, —NH—C(O)—R^{a5}, —NH—S(O)₂—R^{a5}, —P(O)(R^{a5})₂, —S(O)₂—R^{a5}, wherein each of alkyl, alkenyl, alkynyl, heteroalkyl, heteroalkenyl, heteroalkynyl, cycloalkyl, heterocyclyl, aryl, and heteroaryl is optionally substituted with one or more of a group selected from halogen, cyano, hydroxyl, sulfhydryl, —NH₂, —NO₂, alkyl, alkenyl, alkynyl, heteroalkyl, heteroalkenyl, or heteroalkynyl;
R^{a1}, R^{a2} and R^{a3} are each independently selected from the group consisting of hydrogen, hydroxyl, halogen, alkyl, haloalkyl, alkoxyl, cycloalkyl and -alkyl-NH₂;
R^{a4} and R^{a5} are each independently selected from the group consisting of alkyl, alkenyl, alkynyl, heteroalkyl, heteroalkenyl, heteroalkynyl, cycloalkyl, heterocyclyl, aryl and heteroaryl, wherein each of cycloalkyl, heterocyclyl, aryl and heteroaryl is optionally substituted with one or more of a group selected from halogen, cyano, hydroxyl, sulfhydryl, —NH₂, —NO₂, alkyl, alkenyl, alkynyl, heteroalkyl, heteroalkenyl, or heteroalkynyl.

2. A compound of Formula II, 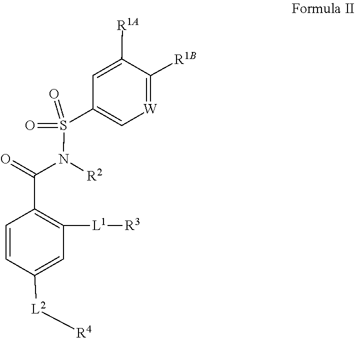
or a tautomer, a stereoisomer, or a pharmaceutically acceptable salt thereof, wherein
W is N or C(R¹);
R^{1A} is selected from the group consisting of hydrogen, halogen, cyano, hydroxyl, sulfhydryl, —NH₂, —NO₂, —SO₂-alkyl, —SO₂-haloalkyl, alkyl, alkenyl, alkynyl, heteroalkyl, heteroalkenyl, heteroalkynyl, haloalkyl, alkoxyl, and haloalkoxyl;
R^{1B} is absent or —NH-L³-R^{a};
R¹, R², L¹, R³, L², R⁴, L³, R^{a} are each as defined in claim 1.

3. The compound, a tautomer, a stereoisomer, or a pharmaceutically acceptable salt thereof, as claimed in claim 2, wherein R^{1A} is —NO₂.

4. The compound, a tautomer, a stereoisomer, or a pharmaceutically acceptable salt thereof, as claimed in claim 2 or 3, wherein R^{1B} is absent.

5. The compound, a tautomer, a stereoisomer, or a pharmaceutically acceptable salt thereof, as claimed in claim 2 or 3, wherein R^{1B} is —NH-L³-R^{a}.

6. The compound, a tautomer, a stereoisomer, or a pharmaceutically acceptable salt thereof, as claimed in claim 5, wherein L³ is alkyl optionally substituted with one or more R^{b}, and each R^{b} is independently selected from the group consisting of halogen, cyano, hydroxyl, sulfhydryl, —NH₂, —NO₂, —SO₂-alkyl, —SO₂-haloalkyl, alkyl, haloalkyl, alkoxyl, and haloalkoxyl.

7. The compound, a tautomer, a stereoisomer, or a pharmaceutically acceptable salt thereof, as claimed in claim 6, wherein L³ is methyl, ethyl or propyl.

8. The compound, a tautomer, a stereoisomer, or a pharmaceutically acceptable salt thereof, as claimed in claim 5, wherein R^{a} is cycloalkyl, heterocyclyl or heteroaryl, wherein each of the cycloalkyl, heterocyclyl and heteroaryl is optionally substituted with one or more R^{c}, wherein each R^{c} is independently selected from the group consisting of hydroxyl, alkyl, haloalkyl, heterocyclyl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3}, wherein R^{a1}, R^{a2} and R^{a3} are each independently selected from the group consisting of hydrogen, hydroxyl, halogen, alkyl, haloalkyl, alkoxyl, cycloalkyl and -alkyl-NH₂.

9. The compound, a tautomer, a stereoisomer, or a pharmaceutically acceptable salt thereof, as claimed in claim 8, wherein R^{a} is a monocyclic heterocyclyl.

10. The compound, a tautomer, a stereoisomer, or a pharmaceutically acceptable salt thereof, as claimed in claim 9, wherein R^{a} is selected from the group consisting of: 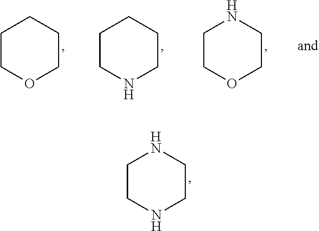
each of which is optionally substituted with one or more R^{c}, and each R^{c} is independently selected from the group consisting of halogen, cyano, hydroxyl, —NH₂, —NO₂, alkyl, heteroalkyl, haloalkyl, alkoxyl, haloalkoxyl, cycloalkyl, heterocyclyl, and —C(O)-alkyl.

11. The compound, a tautomer, a stereoisomer, or a pharmaceutically acceptable salt thereof, as claimed in claim 10, wherein R^{a} is selected from the group consisting of: 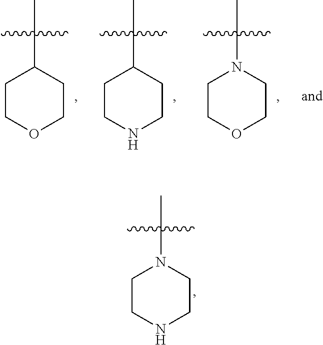
each of which is optionally substituted with one or more R^{c}, and each R^{c} is independently selected from the group consisting of hydroxyl, alkyl, heterocyclyl, and —C(O)-alkyl.

12. The compound, a tautomer, a stereoisomer, or a pharmaceutically acceptable salt thereof, as claimed in claim 10, wherein R^{a} is selected from the group consisting of:

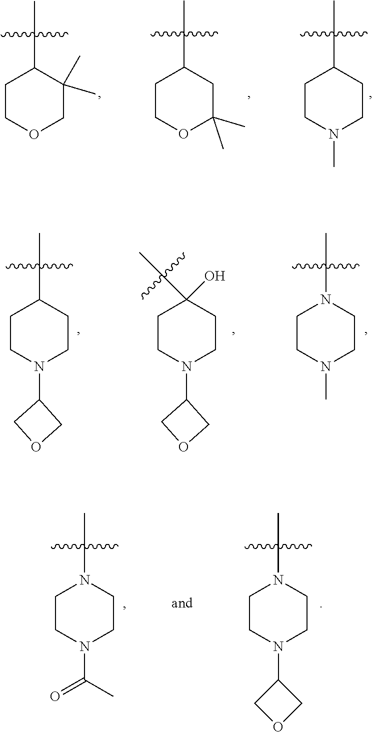

13. The compound, a tautomer, a stereoisomer, or a pharmaceutically acceptable salt thereof, as claimed in claim 8, wherein R^{a} is a polycyclic heterocyclyl or a polycyclic cycloalkyl.

14. The compound, a tautomer, a stereoisomer, or a pharmaceutically acceptable salt thereof, as claimed in claim 13, wherein R^{a} is a spiro-ring system.

15. The compound, a tautomer, a stereoisomer, or a pharmaceutically acceptable salt thereof, as claimed in claim 14, wherein in the spiro-ring system, the number of members of one ring linked to L³ is equal or less than that of the other ring.

16. The compound, a tautomer, a stereoisomer, or a pharmaceutically acceptable salt thereof, as claimed in claim 15, wherein in the spiro-ring system, the ring linked to L³ is a 4-membered ring, and the other ring is a 6-membered ring.

17. The compound, a tautomer, a stereoisomer, or a pharmaceutically acceptable salt thereof, as claimed in claim 14, wherein R^{a} is selected from the group consisting of 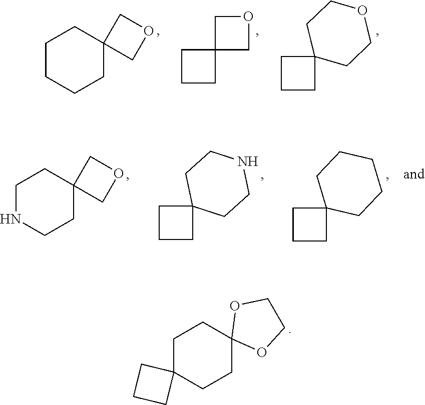
each of which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, haloalkoxyl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3}, and wherein R^{a1}, R^{a2} and R^{a3} are each independently selected from the group consisting of hydrogen, hydroxyl, halogen, alkyl, haloalkyl, alkoxyl, cycloalkyl and -alkyl-NH₂.

18. The compound, a tautomer, a stereoisomer, or a pharmaceutically acceptable salt thereof, as claimed in claim 17, wherein R^{a} is selected from the group consisting of: 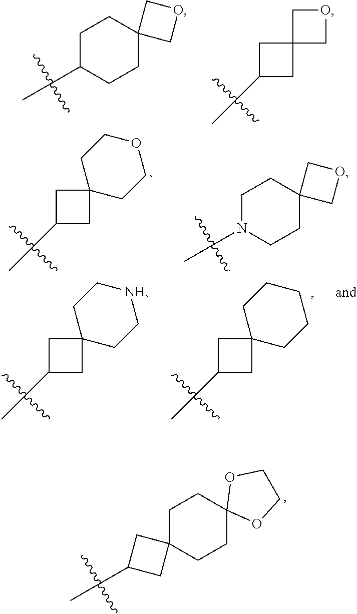
each of which is optionally substituted with one or more R^{c}, and each R^{c} is independently selected from the group consisting of alkyl, haloalkyl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3}, and wherein R^{a1}, R^{a2} and R^{a3} are each independently selected from the group consisting of hydrogen, hydroxyl, halogen, alkyl, haloalkyl, alkoxyl, cycloalkyl and -alkyl-NH₂.

19. The compound, a tautomer, a stereoisomer, or a pharmaceutically acceptable salt thereof, as claimed in claim 17, wherein R^{a} is selected from the group consisting of:

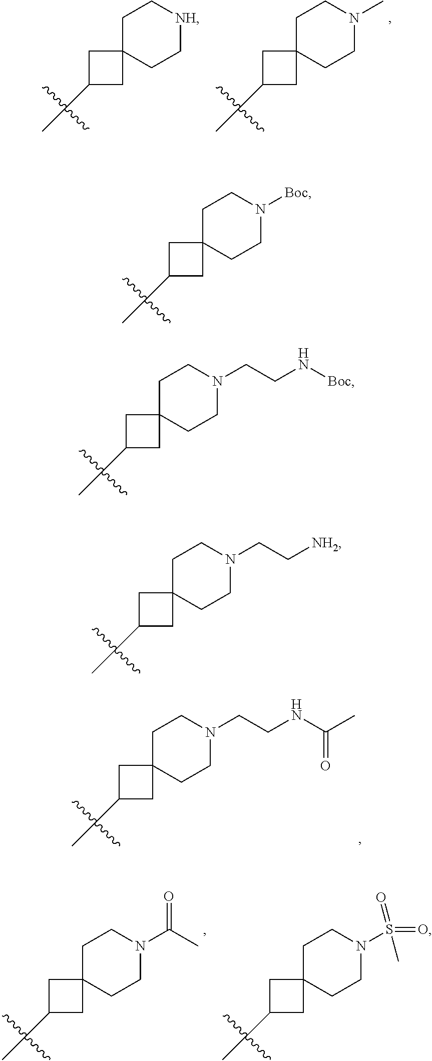

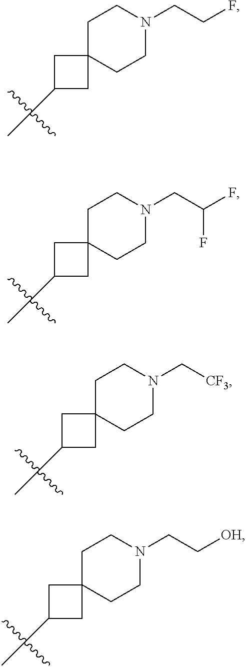 [SMILES: *C1CC2(CCN(CCF)CC2)C1 ; *C1CC2(CCN(CC(F)F)CC2)C1 ; *C1CC2(CCN(CC(F)(F)F)CC2)C1 ; *C1CC2(CCN(CCO)CC2)C1]

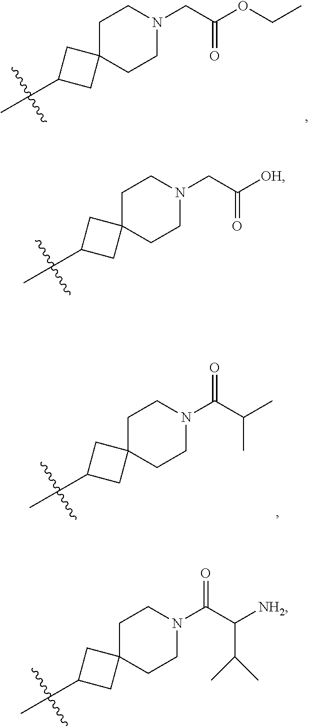 [SMILES: *C1CC2(CCN(CC(=O)OCC)CC2)C1 ; *C1CC2(CCN(CC(=O)O)CC2)C1 ; *C1CC2(CCN(C(=O)C(C)C)CC2)C1 ; *C1CC2(CCN(C(=O)C(N)C(C)C)CC2)C1]

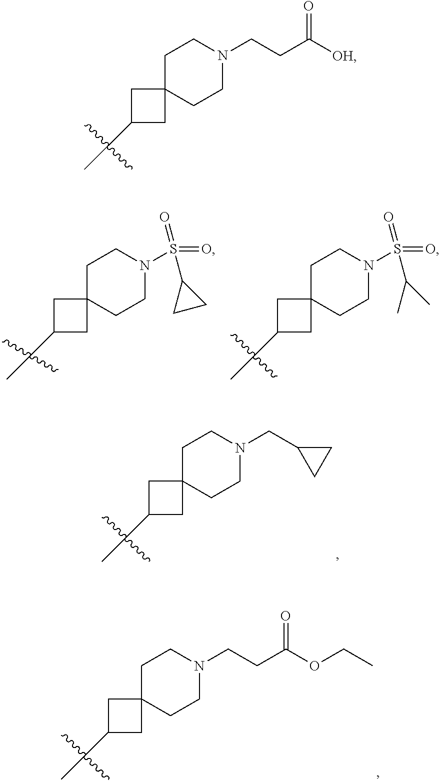 [SMILES: *C1CC2(CCN(CCC(=O)O)CC2)C1 ; *C1CC2(CCN(S(=O)(=O)C3CC3)CC2)C1 ; *C1CC2(CCN(S(=O)(=O)C(C)C)CC2)C1 ; *C1CC2(CCN(CC3CC3)CC2)C1 ; *C1CC2(CCN(CCC(=O)OCC)CC2)C1]

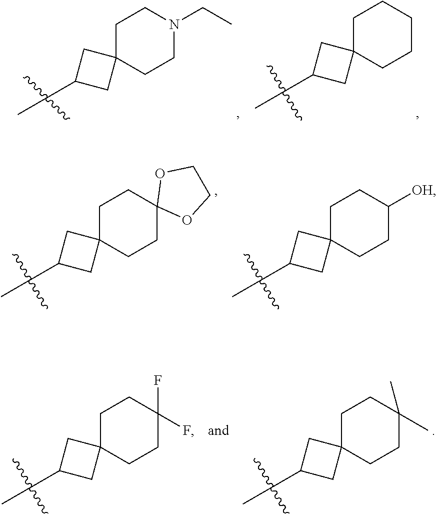 [SMILES: *C1CC2(CCN(CC)CC2)C1 ; *C1CC2(CCCCC2)C1 ; *C1CC2(CCC3(CC2)OCCO3)C1 ; *C1CC2(CCC(O)CC2)C1 ; *C1CC2(CCC(F)(F)CC2)C1 ; *C1CC2(CCC(C)(C)CC2)C1]

20. The compound, a tautomer, a stereoisomer, or a pharmaceutically acceptable salt thereof, as claimed in claim 13, wherein R^{a} is a bridged-ring system.

21. The compound, a tautomer, a stereoisomer, or a pharmaceutically acceptable salt thereof, as claimed in claim 20, wherein R^{a} is selected from the group consisting of 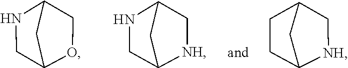 [SMILES: C1OC2CNC1C2 ; C1NC2CNC1C2 ; "and" ; C1CC2CC1CN2]
each of which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, haloalkoxyl, and —C(O)—R^{a1}, wherein R^{a1} is selected from the group consisting of hydrogen, hydroxyl, halogen, alkyl, haloalkyl, and alkoxyl.

22. The compound, a tautomer, a stereoisomer, or a pharmaceutically acceptable salt thereof, as claimed in claim 21, wherein R^{a} is selected from the group consisting of: 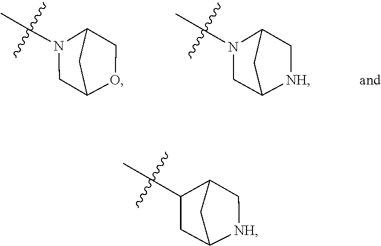 [SMILES: *N1CC2CC1CO2 ; *N1CC2CC1CN2 ; "and" ; *C1CC2CC1CN2]
each of which is optionally substituted with one or more R^{c}, and each R^{c} is independently selected from alkyl or —C(O)—R^{a1}, wherein R^{a1} is selected from the group consisting of hydrogen, hydroxyl, halogen, alkyl, haloalkyl, and alkoxyl.

23. The compound, a tautomer, a stereoisomer, or a pharmaceutically acceptable salt thereof, as claimed in claim 21, wherein R^{a} is selected from the group consisting of:

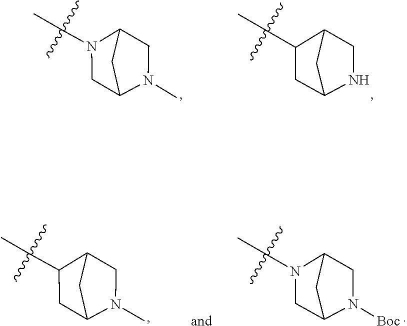

24. The compound, a tautomer, a stereoisomer, or a pharmaceutically acceptable salt thereof, as claimed in claim 13, wherein R^{a} is a fused-ring system.

25. The compound, a tautomer, a stereoisomer, or a pharmaceutically acceptable salt thereof, as claimed in claim 24, wherein R^{a} is 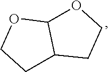
which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, and haloalkoxyl.

26. The compound, a tautomer, a stereoisomer, or a pharmaceutically acceptable salt thereof, as claimed in claim 25, wherein R^{a} is

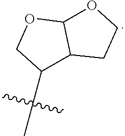 [SMILES: *C1COC2OCCC12]

27. The compound, a tautomer, a stereoisomer, or a pharmaceutically acceptable salt thereof, as claimed in claim 26, wherein R^{a} is

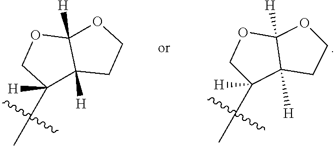 [SMILES: *[C@@H]1CO[C@@H]2OCC[C@@H]21 ; *[C@H]1CO[C@H]2OCC[C@H]21]

28. The compound, a tautomer, a stereoisomer, or a pharmaceutically acceptable salt thereof, as claimed in claim 13, wherein R^{a} is a heteroaryl containing one or more heteroatoms independently selected from O, S, or N atom.

29. The compound, a tautomer, a stereoisomer, or a pharmaceutically acceptable salt thereof, as claimed in claim 28, wherein R^{a} is 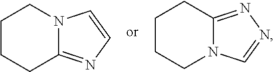 [SMILES: c1cn2c(n1)CCCC2 ; c1nnc2n1CCCC2]
each of which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, and haloalkoxyl.

30. The compound, a tautomer, a stereoisomer, or a pharmaceutically acceptable salt thereof, as claimed in claim 29, wherein R^{a} is 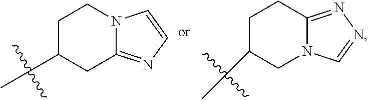 [SMILES: *C1CCn2ccnc2C1 ; *C1CCc2nncn2C1]
each of which is optionally substituted with one or more R^{c}, and each R^{c} is alkyl.

31. The compound, a tautomer, a stereoisomer, or a pharmaceutically acceptable salt thereof, as claimed in claim 30, wherein R^{a} is

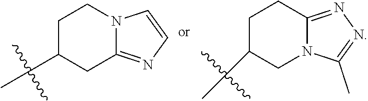 [SMILES: *C1CCn2ccnc2C1 ; *C1CCc2nnc(C)n2C1]

32. The compound, a tautomer, a stereoisomer, or a pharmaceutically acceptable salt thereof, as claimed in any one of the preceding claims, wherein W is CH.

33. The compound, a tautomer, a stereoisomer, or a pharmaceutically acceptable salt thereof, as claimed in any one of the preceding claims, wherein R² is hydrogen.

34. The compound, a tautomer, a stereoisomer, or a pharmaceutically acceptable salt thereof, as claimed in any one of the preceding claims, wherein L¹ is absent or O.

35. The compound, a tautomer, a stereoisomer, or a pharmaceutically acceptable salt thereof, as claimed in any one of the preceding claims, wherein R³ is absent or a heteroaryl optionally substituted with one or more R^{d}.

36. The compound, a tautomer, a stereoisomer, or a pharmaceutically acceptable salt thereof, as claimed in claim 35, wherein R³ is a heteroaryl containing one or more N atoms.

37. The compound, a tautomer, a stereoisomer, or a pharmaceutically acceptable salt thereof, as claimed in claim 36, wherein R³ is

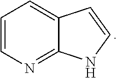

38. The compound, a tautomer, a stereoisomer, or a pharmaceutically acceptable salt thereof, as claimed in claim 37, wherein R³ is

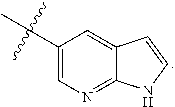 [SMILES: *c1cnc2[nH]ccc2c1]

39. The compound, a tautomer, a stereoisomer, or a pharmaceutically acceptable salt thereof, as claimed in any one of the preceding claims, wherein the -L¹-R³ is absent or

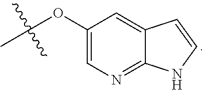 [SMILES: *Oc1cnc2[nH]ccc2c1]

40. The compound, a tautomer, a stereoisomer, or a pharmaceutically acceptable salt thereof, as claimed in any one of the preceding claims, wherein L² is a heterocyclyl optionally substituted with one or more R^{e}.

41. The compound, a tautomer, a stereoisomer, or a pharmaceutically acceptable salt thereof, as claimed in claim 40, wherein L² is a heterocyclyl containing one or more N atoms.

42. The compound, a tautomer, a stereoisomer, or a pharmaceutically acceptable salt thereof, as claimed in claim 41, wherein L² is a group consisting of

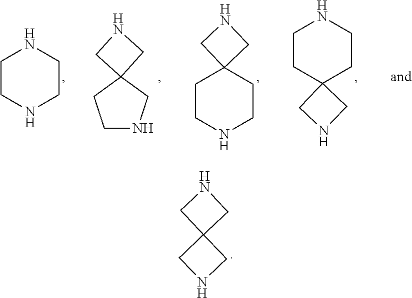

43. The compound, a tautomer, a stereoisomer, or a pharmaceutically acceptable salt thereof, as claimed in claim 42, wherein L² is a group consisting of

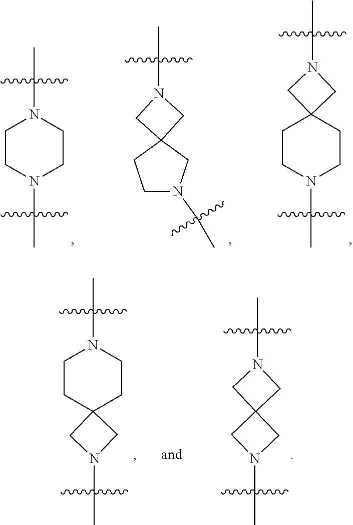 [SMILES: *N1CCN(*)CC1 ; *N1CCC2(C1)CN(*)C2 ; *N1CCC2(CC1)CN(*)C2 ; *N1CCC2(CC1)CN(*)C2 ; *N1CC2(C1)CN(*)C2]

44. The compound, a tautomer, a stereoisomer, or a pharmaceutically acceptable salt thereof, as claimed in any one of the preceding claims, wherein R⁴ is 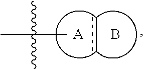
wherein
Ring A is cycloalkyl or heterocyclyl, each of which is optionally substituted with one or more R^{f};
Ring B is aryl, which is optionally substituted with one or more R^{g}; and
R^{f} and R^{g} are each independently as defined in claim 1.

45. The compound, a tautomer, a stereoisomer, or a pharmaceutically acceptable salt thereof, as claimed in claim 44, wherein each R^{f} is independently oxo, alkyl, —S(O)₂-alkyl or —S(O)₂-phenyl, wherein the phenyl is optionally substituted with one or more alkyl; and/or each R^{g} is independently selected from the group consisting of hydroxyl, halogen, —NH₂, —NO₂, —NH—C(O)-alkyl, —NH—S(O)₂-alkyl, —P(O)(alkyl)₂, —S(O)₂-aryl, alkyl, alkenyl, cycloalkyl, aryl and heteroaryl, wherein each of alkyl, aryl and heteroaryl is optionally substituted with one or more groups selected from hydroxyl, halogen or alkyl.

46. The compound, a tautomer, a stereoisomer, or a pharmaceutically acceptable salt thereof, as claimed in any one of the preceding claims, wherein Ring A is a cycloalkyl optionally substituted with one or more R^{f}.

47. The compound, a tautomer, a stereoisomer, or a pharmaceutically acceptable salt thereof, as claimed in claim 46, wherein Ring A is a C₄₋₇ cycloalkyl optionally substituted with one or more R^{f}.

48. The compound, a tautomer, a stereoisomer, or a pharmaceutically acceptable salt thereof, as claimed in claim 47, wherein Ring A is 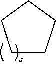
optionally substituted with one or more R^{f}, wherein q is 0, 1, 2 or 3.

49. The compound, a tautomer, a stereoisomer, or a pharmaceutically acceptable salt thereof, as claimed in claim 47, wherein Ring A is 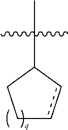
optionally substituted with one or more R^{f}, wherein q is 0, 1, 2 or 3, and [symbol] is the bond via which Ring A is fused to Ring B.

50. The compound, a tautomer, a stereoisomer, or a pharmaceutically acceptable salt thereof, as claimed in any one of claims 46-49, wherein each R^{f} is independently selected from oxo, C₁₋₆ alkyl, —S(O)₂—C₁₋₆ alkyl or —S(O)₂-tolyl.

51. The compound, a tautomer, a stereoisomer, or a pharmaceutically acceptable salt thereof, as claimed in any one of the preceding claims, wherein Ring A is a heterocyclyl optionally substituted with one or more R^{f}.

52. The compound, a tautomer, a stereoisomer, or a pharmaceutically acceptable salt thereof, as claimed in claim 51, wherein Ring A is a 4- to 7-membered heterocyclyl optionally substituted with one or more R^{f}.

53. The compound, a tautomer, a stereoisomer, or a pharmaceutically acceptable salt thereof, as claimed in claim 52, wherein Ring A is 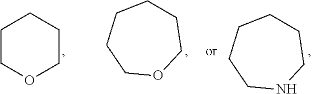 [SMILES: C1CCOCC1 ; C1CCCOCC1 ; C1CCCNCC1]
each of which is optionally substituted with one or more R^{f}.

54. The compound, a tautomer, a stereoisomer, or a pharmaceutically acceptable salt thereof, as claimed in claim 52, wherein Ring A is selected from the group consisting of: 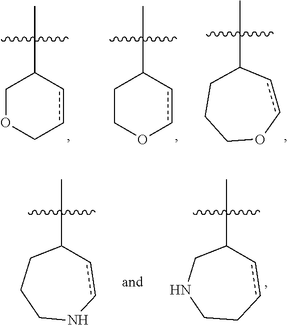
each of which is optionally substituted with one or more R^{f}, and wherein [symbol] is the bond via which Ring A is fused to Ring B.

55. The compound, a tautomer, a stereoisomer, or a pharmaceutically acceptable salt thereof, as claimed in any one of claims 51-54, wherein each R^{f} is independently oxo, C₁₋₆ alkyl, —S(O)₂—C₁₋₆ alkyl or —S(O)₂-tolyl.

56. The compound, a tautomer, a stereoisomer, or a pharmaceutically acceptable salt thereof, as claimed in claim 51, wherein Ring A is selected from the group consisting of: 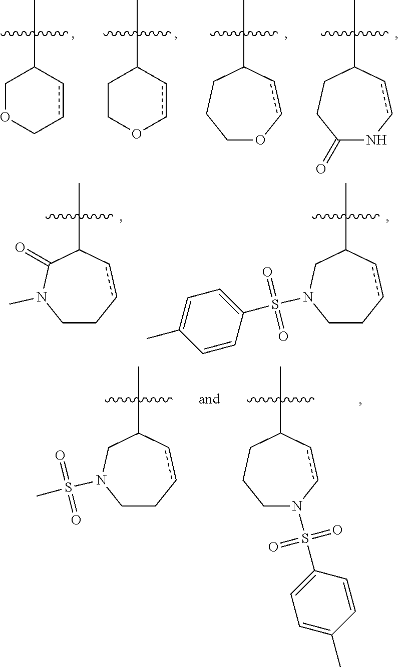
wherein [symbol] is the bond via which Ring A is fused to Ring B.

57. The compound, a tautomer, a stereoisomer, or a pharmaceutically acceptable salt thereof, as claimed in any one of the preceding claims, wherein Ring B is a phenyl, which is optionally substituted with one or more R^{g}, wherein each R^{g} is independently selected from the group consisting of hydroxyl, halogen, —NH₂, —NO₂, —NH—C(O)— alkyl, —NH—S(O)₂-alkyl, —P(O)(alkyl)₂, —S(O)₂-phenyl, alkyl, alkenyl, cycloalkyl, phenyl and heteroaryl, wherein each of alkyl, phenyl and heteroaryl is optionally substituted with one or more group selected from hydroxyl, halogen or alkyl.

58. The compound, a tautomer, a stereoisomer, or a pharmaceutically acceptable salt thereof, as claimed in claim 57, wherein Ring B is an unsubstituted phenyl.

59. The compound, a tautomer, a stereoisomer, or a pharmaceutically acceptable salt thereof, as claimed in claim 57, wherein Ring B is a phenyl substituted with a group selected from the group consisting of hydroxyl, halogen, C₁₋₆ alkyl, —NH₂, —NO₂, cyclopentyl, cyclopentenyl, propenyl, phenyl, pyridinyl, pyrazolyl, thienyl, —NH—C(O)—C₁₋₆ alkyl, —NH—S(O)₂—C₁₋₆ alkyl, —P(O)(C₁₋₆ alkyl)₂, C₁₋₆ alkyl substituted with a hydroxyl, and a phenyl substituted with one or more halogen.

60. The compound, a tautomer, a stereoisomer, or a pharmaceutically acceptable salt thereof, as claimed in claim 59, wherein Ring B is a group selected from the group consisting of: 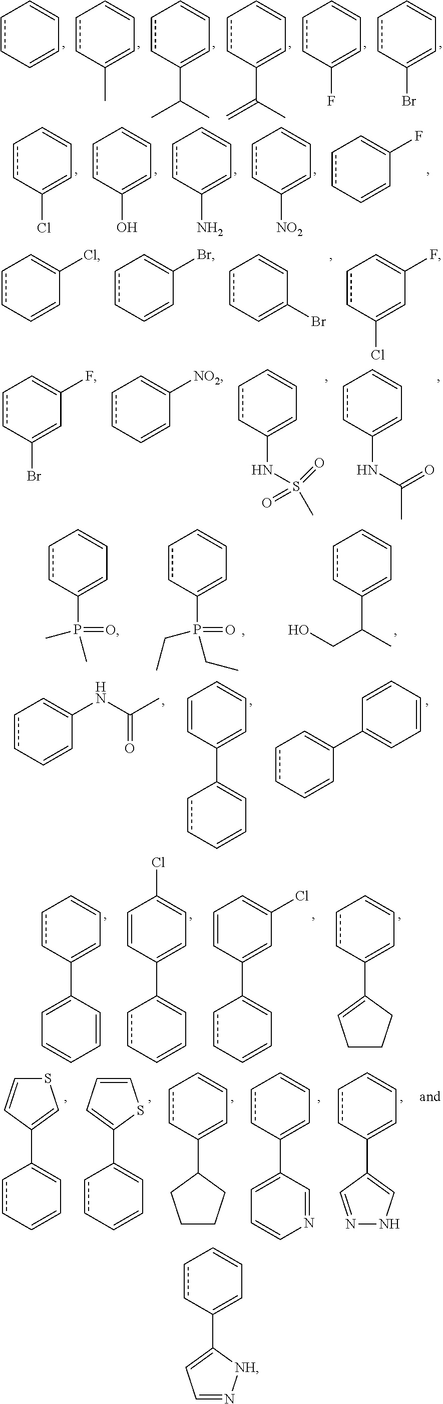
wherein [symbol] is the bond via which Ring B is fused to Ring A.

61. A compound, a tautomer, a stereoisomer, or a pharmaceutically acceptable salt thereof, as claimed in claim 1, wherein the compound having Formula III or Formula IV: 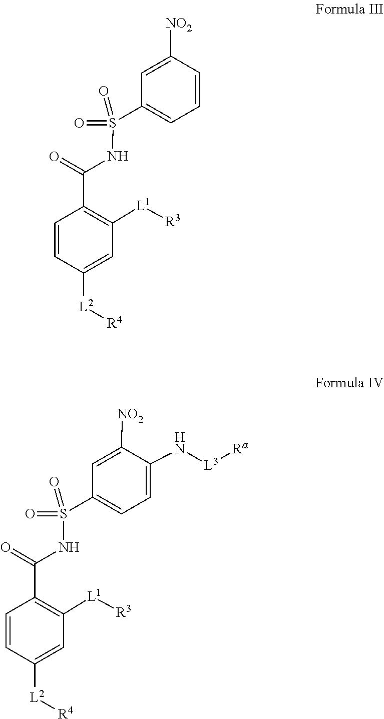
wherein -L¹-R³ is absent or 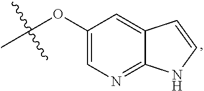 [SMILES: *Oc1cnc2[nH]ccc2c1]
L², L³, R^{a} and R⁴ are as defined in claim 1.

62. The compound, a tautomer, a stereoisomer, or a pharmaceutically acceptable salt thereof, as claimed in claim 61, wherein the compound having Formula IV(a), Formula IV(b), Formula IV(c), Formula IV(d), or Formula IV(e): 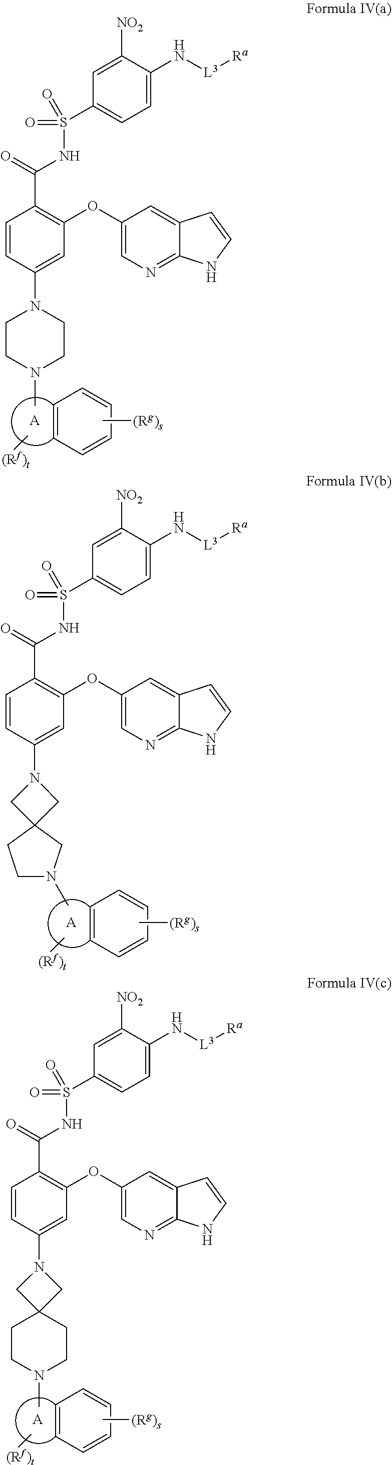 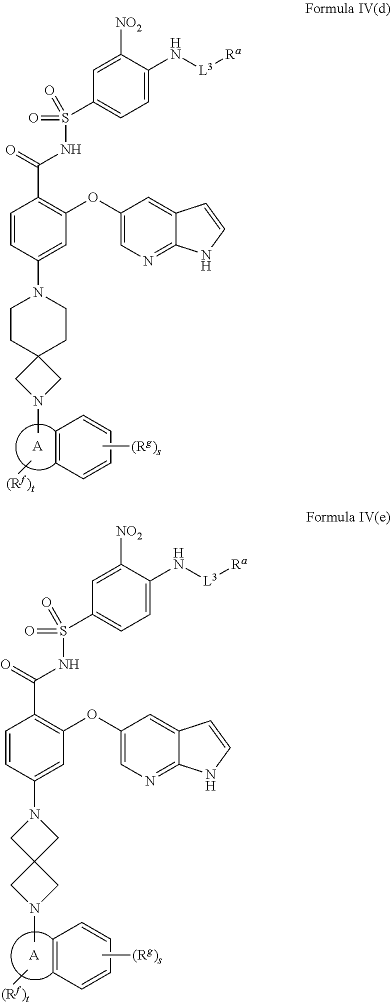
wherein
L³ is alkyl optionally substituted with one or more R^{b}, and each R^{b} is independently selected from the group consisting of halogen, cyano, hydroxyl, —NH₂, —SO₂-alkyl, —SO₂-haloalkyl, alkyl, haloalkyl, alkoxyl, and haloalkoxyl;
R^{a} is independently a cycloalkyl or heterocyclyl;
Ring A is independently a cycloalkyl or heterocyclyl;
each R^{f} is independently oxo, alkyl, —S(O)₂-alkyl or —S(O)₂-phenyl, wherein the phenyl is optionally substituted with one or more alkyl;
each R^{g} is independently selected from the group consisting of hydroxyl, halogen, —NH₂, —NO₂, —NH—C(O)-alkyl, —NH—S(O)₂-alkyl, —P(O)(alkyl)₂, —S(O)₂-aryl, alkyl, alkenyl, cycloalkyl, aryl and heteroaryl, wherein each of alkyl, aryl and heteroaryl is optionally substituted with one or more group selected from hydroxyl, halogen or alkyl; and
each of s and t is independently 0, 1, 2 or 3.

63. The compound, a tautomer, a stereoisomer, or a pharmaceutically acceptable salt thereof, as claimed in claim 62, wherein R^{a} is selected from the group consisting of: 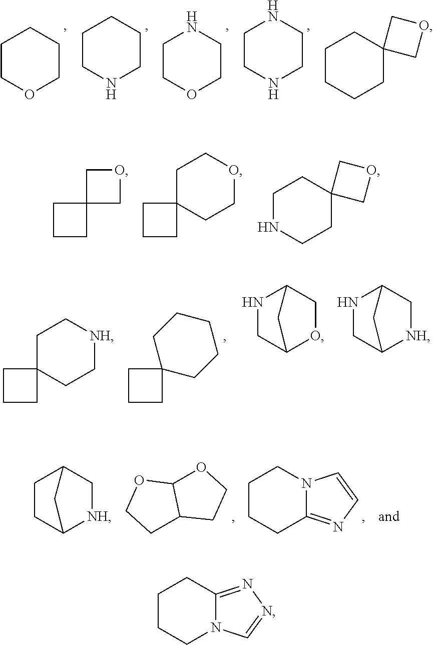
each of which is optionally substituted with one or more R^{c}, and each R^{c} is independently selected from the group consisting of halogen, cyano, hydroxyl, sulfhydryl, —NH₂, —NO₂, alkyl, heteroalkyl, haloalkyl, alkoxyl, haloalkoxyl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3};
R^{a1}, R^{a2} and R^{a3} are each independently selected from the group consisting of hydrogen, hydroxyl, halogen, alkyl, haloalkyl, alkoxyl, cycloalkyl and -alkyl-NH₂.

64. The compound, a tautomer, a stereoisomer, or a pharmaceutically acceptable salt thereof, as claimed in claim 63, wherein R^{a} is selected from the group consisting of: 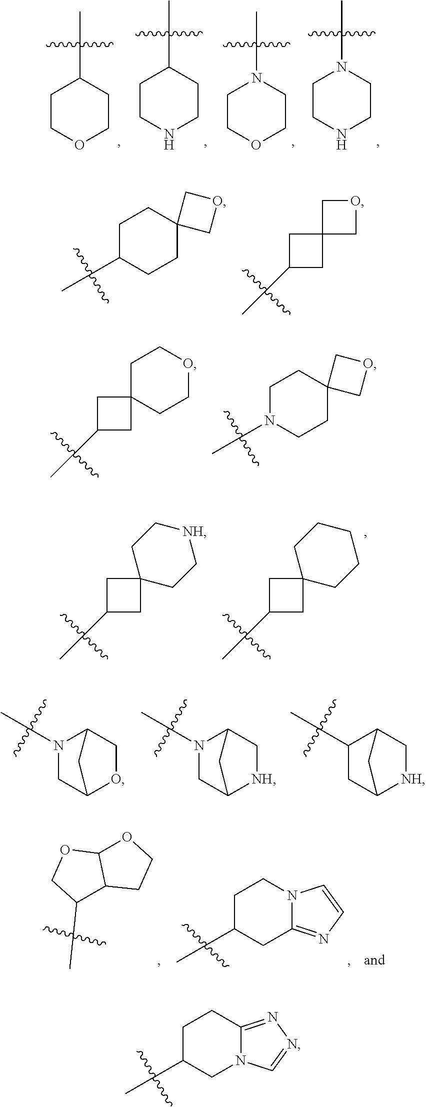
each of which is optionally substituted with one or more R^{c}, wherein each R^{c} is independently selected from the group consisting of halogen, cyano, hydroxyl, sulfhydryl, —NH₂, —NO₂, alkyl, heteroalkyl, haloalkyl, alkoxyl, haloalkoxyl, -alkyl-R^{a1}, -alkyl-C(O)—R^{a1}, —C(O)—R^{a1}, —S(O)₂—R^{a1}, —R^{a2}—NHR^{a3} and —R^{a2}—NHC(O)R^{a3};
R^{a1}, R^{a2} and R^{a3} are each independently selected from the group consisting of hydrogen, hydroxyl, halogen, alkyl, haloalkyl, alkoxyl, cycloalkyl and -alkyl-NH₂.

65. The compound, a tautomer, a stereoisomer, or a pharmaceutically acceptable salt thereof, as claimed in claim 64, wherein R^{a} is selected from the group consisting of:

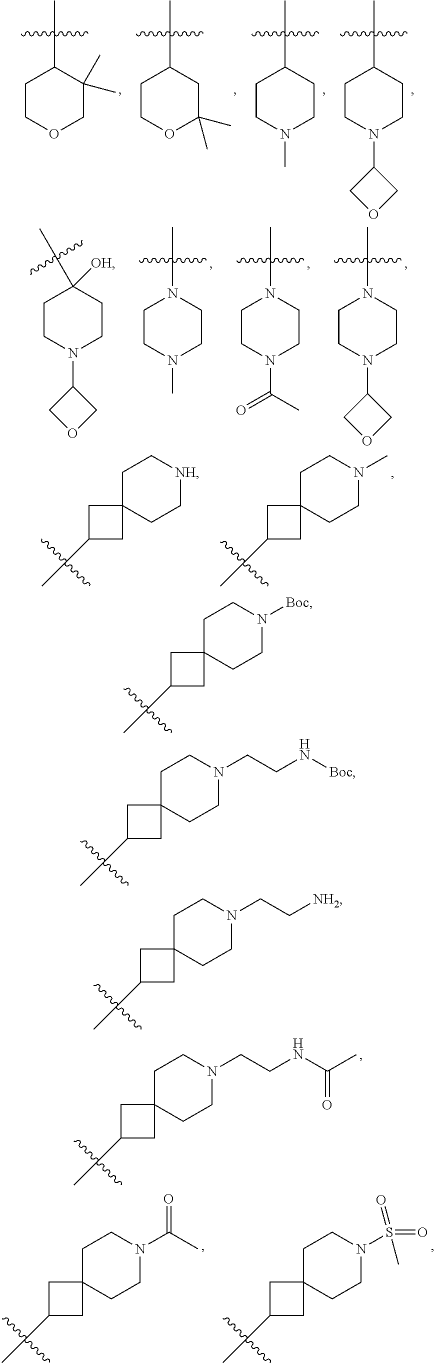

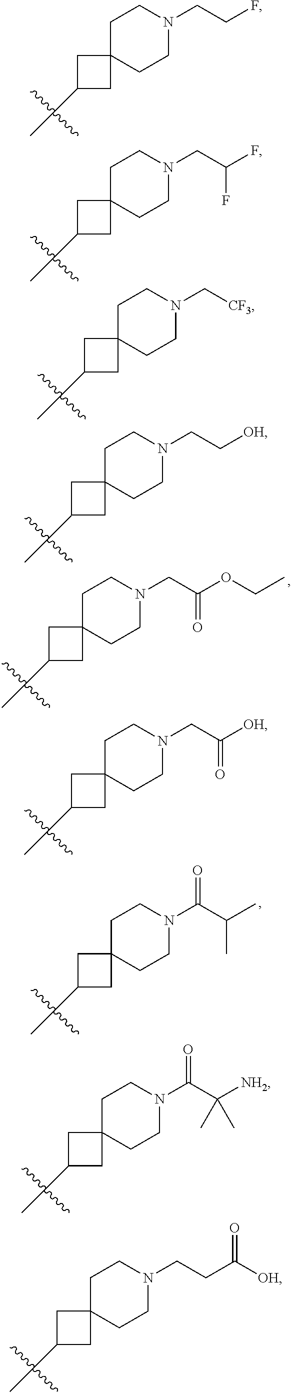

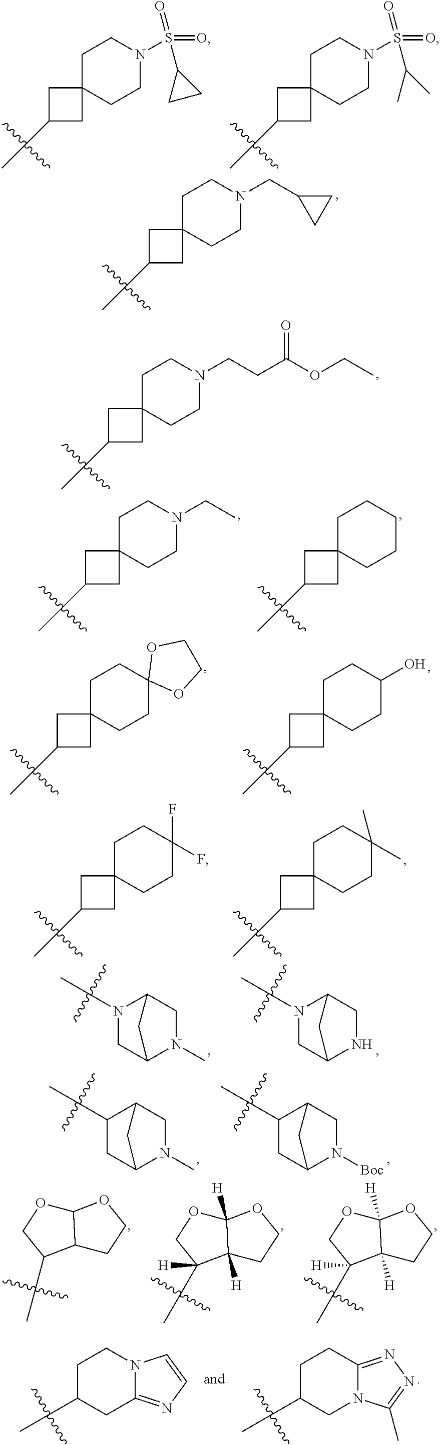

66. The compound, a tautomer, a stereoisomer, or a pharmaceutically acceptable salt thereof, as claimed in any one of claims 62-65, wherein Ring A is selected from 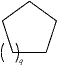
(wherein q is 0, 1, 2 or 3), 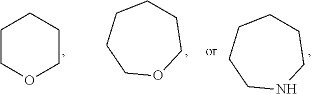 [SMILES: C1CCOCC1 ; C1CCCOCC1 ; C1CCCNCC1]
each of which is optionally substituted with one or more R, wherein each R^{f} is independently oxo, C₁₋₆ alkyl, —S(O)₂—C₁₋₆ alkyl or —S(O)₂-tolyl.

67. The compound, a tautomer, a stereoisomer, or a pharmaceutically acceptable salt thereof, as claimed in claim 64, wherein Ring A is selected from the group consisting of: 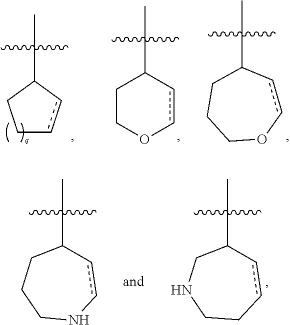
wherein q is 0, 1, 2 or 3, and [symbol] is the bond via which Ring A is fused to Ring B.

68. The compound, a tautomer, a stereoisomer, or a pharmaceutically acceptable salt thereof, as claimed in claim 67, wherein Ring A is selected from the group consisting of: 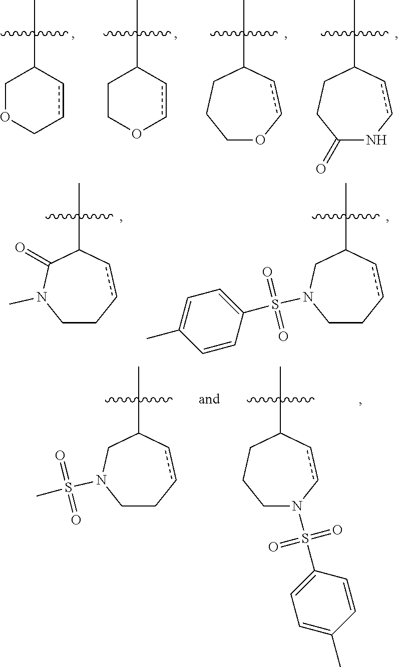
wherein [symbol] is the bond via which Ring A is fused to Ring B.

69. The compound, a tautomer, a stereoisomer, or a pharmaceutically acceptable salt thereof, as claimed in any one of claims 62-68, wherein
each R^{f}, when present, is independently oxo, C₁₋₆ alkyl, —S(O)₂—C₁₋₆ alkyl or —S(O)₂-tolyl;
each R^{g}, when present, is independently selected from a group consisting of hydroxyl, halogen, C₁₋₆ alkyl, —NH₂, —NO₂, cyclopentyl, cyclopentenyl, propenyl, phenyl, pyridinyl, pyrazolyl, thienyl, —NH—C(O)—C₁₋₆ alkyl, —NH—S(O)₂—C₁₋₆ alkyl, —P(O)(C₁₋₆ alkyl)₂, C₁₋₆ alkyl substituted with a hydroxyl, and a phenyl substituted with one or more halogen.

70. The compound, a tautomer, a stereoisomer, or a pharmaceutically acceptable salt thereof, as claimed in any one of claims 62-69, wherein each R^{f}, when present, is independently oxo, methyl, —S(O)₂-methyl or —S(O)₂-tolyl.

71. The compound, a tautomer, a stereoisomer, or a pharmaceutically acceptable salt thereof, as claimed in any one of claims 62-70, wherein each R^{g}, when present, is independently selected from the group consisting of hydroxyl, halogen, C₁₋₆ alkyl, —NH₂, —NO₂, cyclopentyl, cyclopentenyl, propenyl, phenyl, pyridinyl, pyrazolyl, thienyl, —NH—C(O)—C₁₋₆ alkyl, —NH—S(O)₂—C₁₋₆ alkyl, —P(O)(C₁₋₆ alkyl)₂, C₁₋₆ alkyl substituted with a hydroxyl, and a phenyl substituted with one or more halogen.

72. The compound, a tautomer, a stereoisomer, or a pharmaceutically acceptable salt thereof, as claimed in any one of claims 62-71, wherein each R^{g}, when present, is independently selected from the group consisting of hydroxyl, halogen, —NH₂, —NO₂, methyl, isopropyl, propenyl, cyclopentyl, cyclopentenyl, phenyl, pyridinyl, pyrazolyl, thienyl, —NH—C(O)-methyl, —NH—S(O)₂-methyl, —P(O)(C₁₋₂ alkyl)₂, —CH(CH₃)CH₂OH, and chlorophenyl.

73. The compound, a tautomer, a stereoisomer, or a pharmaceutically acceptable salt thereof, as claimed in any one of claims 62-72, wherein each R^{g} is independently a halogen selected from F, Cl, Br, or I.

74. A compound selected from the group consisting of:

| Compound | |
|---|---|
| No. | IUPAC Name |
| 1 | 2-((1H-pyrrolo[2,3-b]pyridin-5-yl)oxy)-N-((3-nitrophenyl)sulfonyl)-4- |
| | (4-(1,2,3,4-tetrahydronaphthalen-1-yl)piperazin-1-yl)benzamide |
| 1A | (R)-2-((1H-pyrrolo[2,3-b]pyridin-5-yl)oxy)-N-((3- |
| | nitrophenyl)sulfonyl)-4-(4-(1,2,3,4-tetrahydronaphthalen-1- |
| | yl)piperazin-1-yl)benzamide |
| 1B | (S)-2-((1H-pyrrolo[2,3-b]pyridin-5-yl)oxy)-N-((3- |
| | nitrophenyl)sulfonyl)-4-(4-(1,2,3,4-tetrahydronaphthalen-1- |
| | yl)piperazin-1-yl)benzamide |
| 2 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(1,2,3,4- |
| | tetrahydronaphthalen-1-yl)piperazin-1-yl)benzamide |
| 3 | 2-((1H-pyrrolo[2,3-b]pyridin-5-yl)oxy)-4-(4-(2,3-dihydro-1H-inden-1- |
| | yl)piperazin-1-yl)-N-((3-nitrophenyl)sulfonyl)benzamide |
| 3A | (S)-2-((1H-pyrrolo[2,3-b]pyridin-5-yl)oxy)-4-(4-(2,3-dihydro-1H- |
| | inden-1-yl)piperazin-1-yl)-N-((3-nitrophenyl)sulfonyl)benzamide |
| 3B | (R)-2-((1H-pyrrolo[2,3-b]pyridin-5-yl)oxy)-4-(4-(2,3-dihydro-1H- |
| | inden-1-yl)piperazin-1-yl)-N-((3-nitrophenyl)sulfonyl)benzamide |
| 4 | 2-((1H-pyrrolo[2,3-b]pyridin-5-yl)oxy)-4-(4-(2,3-dihydro-1H-inden-1- |
| | yl)piperazin-1-yl)-N-((3-nitro-4-(((tetrahydro-2H-pyran-4- |
| | yl)methyl)amino)phenyl)sulfonyl)benzamide |
| 5 | 2-((1H-pyrrolo[2,3-b]pyridin-5-yl)oxy)-N-((3-nitrophenyl)sulfonyl)-4- |
| | (4-(6,7,8,9-tetrahydro-5H-benzo[7]annulen-5-yl)piperazin-1- |
| | yl)benzamide |
| 5A | (S)-2-((1H-pyrrolo[2,3-b]pyridin-5-yl)oxy)-N-((3- |
| | nitrophenyl)sulfonyl)-4-(4-(6,7,8,9-tetrahydro-5H-benzo[7]annulen-5- |
| | yl)piperazin-1-yl)benzamide |
| 5B | (R)-2-((1H-pyrrolo[2,3-b]pyridin-5-yl)oxy)-N-((3- |
| | nitrophenyl)sulfonyl)-4-(4-(6,7,8,9-tetrahydro-5H-benzo[7]annulen-5- |
| | yl)piperazin-1-yl)benzamide |
| 6 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(6,7,8,9- |
| | tetrahydro-5H-benzo[7]annulen-5-yl)piperazin-1-yl)benzamide |
| 6A | (S)-2-((1H-pyrrolo[2,3-b]pyridin-5-yl)oxy)-N-((3-nitro-4- |
| | (((tetrahydro-2H-pyran-4-yl)methyl)amino)phenyl)sulfonyl)-4-(4- |
| | (6,7,8,9-tetrahydro-5H-benzo[7]annulen-5-yl)piperazin-1- |
| | yl)benzamide |
| 6B | (R)-2-((1H-pyrrolo[2,3-b]pyridin-5-yl)oxy)-N-((3-nitro-4- |
| | (((tetrahydro-2H-pyran-4-yl)methyl)amino)phenyl)sulfonyl)-4-(4- |
| | (6,7,8,9-tetrahydro-5H-benzo[7]annulen-5-yl)piperazin-1- |
| | yl)benzamide |
| 7 | 2-((1H-pyrrolo[2,3-b]pyridin-5-yl)oxy)-N-((3-nitrophenyl)sulfonyl)-4- |
| | (4-(7-phenyl-2,3-dihydro-1H-inden-1-yl)piperazin-1-yl)benzamide |
| 8 | 2-((1H-pyrrolo[2,3-b]pyridin-5-yl)oxy)-N-((3-nitrophenyl)sulfonyl)-4- |
| | (4-(8-phenyl-1,2,3,4-tetrahydronaphthalen-1-yl)piperazin-1- |
| | yl)benzamide |
| 9 | 2-((1H-pyrrolo[2,3-b]pyridin-5-yl)oxy)-4-(4-(chroman-4-yl)piperazin- |
| | 1-yl)-N-((3-nitrophenyl)sulfonyl)benzamide |
| 10 | 2-((1H-pyrrolo[2,3-b]pyridin-5-yl)oxy)-4-(4-(isochroman-4- |
| | yl)piperazin-1-yl)-N-((3-nitrophenyl)sulfonyl)benzamide |
| 11 | 2-((1H-pyrrolo[2,3-b]pyridin-5-yl)oxy)-N-((3-nitrophenyl)sulfonyl)-4- |
| | (4-(8-(thiophen-3-yl)-1,2,3,4-tetrahydronaphthalen-1-yl)piperazin-1- |
| | yl)benzamide |
| 12 | 2-((1H-pyrrolo[2,3-b]pyridin-5-yl)oxy)-4-(4-(isochroman-4- |
| | yl)piperazin-1-yl)-N-((3-nitro-4-(((tetrahydro-2H-pyran-4- |
| | yl)methyl)amino)phenyl)sulfonyl)benzamide |
| 13 | 2-((1H-pyrrolo[2,3-b]pyridin-5-yl)oxy)-4-(4-(8-(4-chlorophenyl)- |
| | 1,2,3,4-tetrahydronaphthalen-1-yl)piperazin-1-yl)-N-((3- |
| | nitrophenyl)sulfonyl)benzamide |
| 14 | 2-((1H-pyrrolo[2,3-b]pyridin-5-yl)oxy)-4-(4-(8-(3-chlorophenyl)- |
| | 1,2,3,4-tetrahydronaphthalen-1-yl)piperazin-1-yl)-N-((3- |
| | nitrophenyl)sulfonyl)benzamide |
| 15 | 2-((1H-pyrrolo[2,3-b]pyridin-5-yl)oxy)-N-((3-nitrophenyl)sulfonyl)-4- |
| | (4-(8-(thiophen-2-yl)-1,2,3,4-tetrahydronaphthalen-1-yl)piperazin-1- |
| | yl)benzamide |
| 16 | 2-((1H-pyrrolo[2,3-b]pyridin-5-yl)oxy)-4-(4-(8-(4-chlorophenyl)- |
| | 1,2,3,4-tetrahydronaphthalen-1-yl)piperazin-1-yl)-N-((3-nitro-4- |
| | (((tetrahydro-2H-pyran-4-yl)methyl)amino)phenyl)sulfonyl)benzamide |
| 17 | 2-((1H-pyrrolo[2,3-b]pyridin-5-yl)oxy)-4-(4-(chroman-4-yl)piperazin- |
| | 1-yl)-N-((3-nitro-4-(((tetrahydro-2H-pyran-4- |
| | yl)methyl)amino)phenyl)sulfonyl)benzamide |
| 18 | 2-((1H-pyrrolo[2,3-b]pyridin-5-yl)oxy)-4-(4-(1-bromo-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 18A | (R)-2-((1H-pyrrolo[2,3-b]pyridin-5-yl)oxy)-4-(4-(1-bromo-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 18B | (S)-2-((1H-pyrrolo[2,3-b]pyridin-5-yl)oxy)-4-(4-(1-bromo-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 19 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(1-phenyl-6,7,8,9- |
| | tetrahydro-5H-benzo[7]annulen-5-yl)piperazin-1-yl)benzamide |
| 20 | 2-((1H-pyrrolo[2,3-b]pyridin-5-yl)oxy)-4-(4-(1-methyl-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 21 | 2-((1H-pyrrolo[2,3-b]pyridin-5-yl)oxy)-4-(4-(3-bromo-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 22 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(3-phenyl-6,7,8,9- |
| | tetrahydro-5H-benzo[7]annulen-5-yl)piperazin-1-yl)benzamide |
| 23 | 2-((1H-pyrrolo[2,3-b]pyridin-5-yl)oxy)-4-(4-(2-bromo-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 24 | (R)-N-((4-(((7-(2-acetamidoethyl)-7-azaspiro[3.5]nonan-2- |
| | yl)methyl)amino)-3-nitrophenyl)sulfonyl)-4-(4-(1-chloro-6,7,8,9- |
| | tetrahydro-5H-benzo[7]annulen-5-yl)piperazin-1-yl)benzamide |
| 25 | 2-((1H-pyrrolo[2,3-b]pyridin-5-yl)oxy)-4-(4-(2-chloro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 26 | 2-((1H-pyrrolo[2,3-b]pyridin-5-yl)oxy)-4-(4-(3-chloro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 27 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 27A | (S)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 27B | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 28 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(2-oxo-2,3,4,5- |
| | tetrahydro-1H-benzo[b]azepin-5-yl)piperazin-1-yl)benzamide |
| 29 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((1- |
| | methylpiperidin-4-yl)methyl)amino)-3- |
| | nitrophenyl)sulfonyl)benzamide |
| 30 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(3-nitro-6,7,8,9- |
| | tetrahydro-5H-benzo[7]annulen-5-yl)piperazin-1-yl)benzamide |
| 31 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-((2- |
| | morpholinoethyl)amino)-3-nitrophenyl)sulfonyl)benzamide |
| 31A | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-((2- |
| | morpholinoethyl)amino)-3-nitrophenyl)sulfonyl)benzamide |
| 31B | (S)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-((2- |
| | morpholinoethyl)amino)-3-nitrophenyl)sulfonyl)benzamide |
| 32 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-((2-(4- |
| | methylpiperazin-1-yl)ethyl)amino)-3-nitrophenyl)sulfonyl)benzamide |
| 33 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(1-nitro-6,7,8,9- |
| | tetrahydro-5H-benzo[7]annulen-5-yl)piperazin-1-yl)benzamide |
| 34 | 2-((1H-pyrrolo[2,3-b]pyridin-5-yl)oxy)-4-(4-(3-acetamido-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 35 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(3-tosyl-2,3,4,5- |
| | tetrahydro-1H-benzo[d]azepin-1-yl)piperazin-1-yl)benzamide |
| 36 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(2,3,4,5- |
| | tetrahydrobenzo[b]oxepin-5-yl)piperazin-1-yl)benzamide |
| 37 | 2-((1H-pyrrolo[2,3-b]pyridin-5-yl)oxy)-4-(4-(3-fluoro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 38 | 2-((1H-pyrrolo[2,3-b]pyridin-5-yl)oxy)-N-((4-(((2-oxaspiro[3.5]nonan- |
| | 7-yl)methyl)amino)-3-nitrophenyl)sulfonyl)-4-(4-(1-chloro-6,7,8,9- |
| | tetrahydro-5H-benzo[7]annulen-5-yl)piperazin-1-yl)benzamide |
| 39 | 2-((1H-pyrrolo[2,3-b]pyridin-5-yl)oxy)-N-((4-((2-(4-acetylpiperazin-1- |
| | yl)ethyl)amino)-3-nitrophenyl)sulfonyl)-4-(4-(1-chloro-6,7,8,9- |
| | tetrahydro-5H-benzo[7]annulen-5-yl)piperazin-1-yl)benzamide |
| 40 | (R)-2-((1H-pyrrolo[2,3-b]pyridin-5-yl)oxy)-4-(4-(1-hydroxy-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 41 | 2-((1H-pyrrolo[2,3-b]pyridin-5-yl)oxy)-N-((4-((2-(2-oxa-5- |
| | azabicyclo[2.2.1]heptan-5-yl)ethyl)amino)-3-nitrophenyl)sulfonyl)-4- |
| | (4-(1-chloro-6,7,8,9-tetrahydro-5H-benzo[7]annulen-5-yl)piperazin-1- |
| | yl)benzamide |
| 42 | 2-((1H-pyrrolo[2,3-b]pyridin-5-yl)oxy)-N-((4-((2-(2-oxa-7- |
| | azaspiro[3.5]nonan-7-yl)ethyl)amino)-3-nitrophenyl)sulfonyl)-4-(4-(1- |
| | chloro-6,7,8,9-tetrahydro-5H-benzo[7]annulen-5-yl)piperazin-1- |
| | yl)benzamide |
| 43 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-((2-(5- |
| | methyl-2,5-diazabicyclo[2.2.1]heptan-2-yl)ethyl)amino)-3- |
| | nitrophenyl)sulfonyl)benzamide |
| 44 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((2- |
| | methyl-2-azabicyclo[2.2.1]heptan-5-yl)methyl)amino)-3- |
| | nitrophenyl)sulfonyl)benzamide |
| 45 | 2-((1H-pyrrolo[2,3-b]pyridin-5-yl)oxy)-4-(4-(3-methyl-2-oxo-2,3,4,5- |
| | tetrahydro-1H-benzo[d]azepin-1-yl)piperazin-1-yl)-N-((3-nitro-4- |
| | (((tetrahydro-2H-pyran-4-yl)methyl)amino)phenyl)sulfonyl)benzamide |
| 46 | 2-((1H-pyrrolo[2,3-b]pyridin-5-yl)oxy)-N-((4-(((2- |
| | oxaspiro[3.3]heptan-6-yl)methyl)amino)-3-nitrophenyl)sulfonyl)-4-(4- |
| | (1-chloro-6,7,8,9-tetrahydro-5H-benzo[7]annulen-5-yl)piperazin-1- |
| | yl)benzamide |
| 47 | 2-((1H-pyrrolo[2,3-b]pyridin-5-yl)oxy)-4-(4-(1-(cyclopent-1-en-1-yl)- |
| | 6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 48 | (R)-4-(4-(1-chloro-6,7,8,9-tetrahydro-5H-benzo[7]annulen-5- |
| | yl)piperazin-1-yl)-N-((4-(((7-(methylsulfonyl)-7-azaspiro[3.5]nonan-2- |
| | yl)methyl)amino)-3-nitrophenyl)sulfonyl)benzamide |
| 49 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(1-(pyridin-3-yl)- |
| | 6,7,8,9-tetrahydro-5H-benzo[7]annulen-5-yl)piperazin-1-yl)benzamide |
| 50 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(1-(prop-1-en-2- |
| | yl)-6,7,8,9-tetrahydro-5H-benzo[7]annulen-5-yl)piperazin-1- |
| | yl)benzamide |
| 51 | 2-((1H-pyrrolo[2,3-b]pyridin-5-yl)oxy)-4-(4-(1-isopropyl-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 52 | 4-(4-(1-(1H-pyrazol-4-yl)-6,7,8,9-tetrahydro-5H-benzo[7]annulen-5- |
| | yl)piperazin-1-yl)-2-((1H-pyrrolo[2,3-b]pyridin-5-yl)oxy)-N-((3-nitro- |
| | 4-(((tetrahydro-2H-pyran-4- |
| | yl)methyl)amino)phenyl)sulfonyl)benzamide |
| 53 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((5,6,7,8-tetrahydroimidazo[1,2-a]pyridin-7- |
| | yl)methyl)amino)phenyl)sulfonyl)benzamide |
| 54 | 4-(4-(1-(1H-pyrazol-3-yl)-6,7,8,9-tetrahydro-5H-benzo[7]annulen-5- |
| | yl)piperazin-1-yl)-2-((1H-pyrrolo[2,3-b]pyridin-5-yl)oxy)-N-((3-nitro- |
| | 4-(((tetrahydro-2H-pyran-4- |
| | yl)methyl)amino)phenyl)sulfonyl)benzamide |
| 55 | 2-((1H-pyrrolo[2,3-b]pyridin-5-yl)oxy)-4-(4-(1-cyclopentyl-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 56 | 2-((1H-pyrrolo[2,3-b]pyridin-5-yl)oxy)-4-(4-(1-(methylsulfonamido)- |
| | 6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 57 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((3- |
| | methyl-5,6,7,8-tetrahydro-[1,2,4]triazolo[4,3-a]pyridin-6- |
| | yl)methyl)amino)-3-nitrophenyl)sulfonyl)benzamide |
| 58 | 2-((1H-pyrrolo[2,3-b]pyridin-5-yl)oxy)-4-(4-(1-amino-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 59 | 2-((1H-pyrrolo[2,3-b]pyridin-5-yl)oxy)-4-(4-(1-acetamido-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 60 | 2-((1H-pyrrolo[2,3-b]pyridin-5-yl)oxy)-4-(4-(3-(methylsulfonyl)- |
| | 2,3,4,5-tetrahydro-1H-benzo[d]azepin-1-yl)piperazin-1-yl)-N-((3-nitro- |
| | 4-(((tetrahydro-2H-pyran-4- |
| | yl)methyl)amino)phenyl)sulfonyl)benzamide |
| 61 | 2-((1H-pyrrolo[2,3-b]pyridin-5-yl)oxy)-4-(4-(1-(1-hydroxypropan-2- |
| | yl)-6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 62 | 2-((1H-pyrrolo[2,3-b]pyridin-5-yl)oxy)-4-(4-(1-(diethylphosphoryl)- |
| | 6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 63 | 2-((1H-pyrrolo[2,3-b]pyridin-5-yl)oxy)-4-(4-(1-(dimethylphosphoryl)- |
| | 6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 64 | 2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4- |
| | (((hexahydrofuro[2,3-b]furan-3-yl)methyl)amino)-3- |
| | nitrophenyl)sulfonyl)benzamide |
| 65 | (R)-2-((1H-pyrrolo[2,3-b]pyridin-5-yl)oxy)-N-((4-((2-(4- |
| | acetylpiperazin-1-yl)ethyl)amino)-3-nitrophenyl)sulfonyl)-4-(4-(1- |
| | bromo-6,7,8,9-tetrahydro-5H-benzo[7]annulen-5-yl)piperazin-1- |
| | yl)benzamide |
| 66 | (R)-2-((1H-pyrrolo[2,3-b]pyridin-5-yl)oxy)-4-(4-(1-bromo-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 67 | (R)-2-((1H-pyrrolo[2,3-b]pyridin-5-yl)oxy)-N-((4-(((2- |
| | oxaspiro[3.5]nonan-7-yl)methyl)amino)-3-nitrophenyl)sulfonyl)-4-(4- |
| | (1-bromo-6,7,8,9-tetrahydro-5H-benzo[7]annulen-5-yl)piperazin-1- |
| | yl)benzamide |
| 68 | (R)-2-((1H-pyrrolo[2,3-b]pyridin-5-yl)oxy)-N-((4-((2-(2-oxa-7- |
| | azaspiro[3.5]nonan-7-yl)ethyl)amino)-3-nitrophenyl)sulfonyl)-4-(4-(1- |
| | bromo-6,7,8,9-tetrahydro-5H-benzo[7]annulen-5-yl)piperazin-1- |
| | yl)benzamide |
| 69 | (R)-2-((1H-pyrrolo[2,3-b]pyridin-5-yl)oxy)-4-(4-(1-bromo-6,7,8,9- |
| | tetrahydro-5H-benzo[7]annulen-5-yl)piperazin-1-yl)-N-((4-(((1- |
| | methylpiperidin-4-yl)methyl)amino)-3- |
| | nitrophenyl)sulfonyl)benzamide |
| 70 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 71 | (R)-2-((1H-pyrrolo[2,3-b]pyridin-5-yl)oxy)-N-((4-(((2- |
| | oxaspiro[3.5]nonan-7-yl)methyl)amino)-3-nitrophenyl)sulfonyl)-4-(4- |
| | (1-chloro-6,7,8,9-tetrahydro-5H-benzo[7]annulen-5-yl)piperazin-1- |
| | yl)benzamide |
| 72 | (R)-2-((1H-pyrrolo[2,3-b]pyridin-5-yl)oxy)-N-((4-(((2- |
| | oxaspiro[3.3]heptan-6-yl)methyl)amino)-3-nitrophenyl)sulfonyl)-4-(4- |
| | (1-chloro-6,7,8,9-tetrahydro-5H-benzo[7]annulen-5-yl)piperazin-1- |
| | yl)benzamide |
| 73 | (R)-2-((1H-pyrrolo[2,3-b]pyridin-5-yl)oxy)-N-((3-nitro-4- |
| | (((tetrahydro-2H-pyran-4-yl)methyl)amino)phenyl)sulfonyl)-4-(4-(1- |
| | nitro-6,7,8,9-tetrahydro-5H-benzo[7]annulen-5-yl)piperazin-1- |
| | yl)benzamide |
| 74 | (R)-2-((1H-pyrrolo[2,3-b]pyridin-5-yl)oxy)-N-((4-(((2- |
| | oxaspiro[3.5]nonan-7-yl)methyl)amino)-3-nitrophenyl)sulfonyl)-4-(4- |
| | (1-nitro-6,7,8,9-tetrahydro-5H-benzo[7]annulen-5-yl)piperazin-1- |
| | yl)benzamide |
| 75 | (R)-2-((1H-pyrrolo[2,3-b]pyridin-5-yl)oxy)-N-((4-(((2- |
| | oxaspiro[3.3]heptan-6-yl)methyl)amino)-3-nitrophenyl)sulfonyl)-4-(4- |
| | (1-nitro-6,7,8,9-tetrahydro-5H-benzo[7]annulen-5-yl)piperazin-1- |
| | yl)benzamide |
| 76 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(4-(1-tosyl-2,3,4,5- |
| | tetrahydro-1H-benzo[b]azepin-5-yl)piperazin-1-yl)benzamide |
| 77 | (R)-2-((1H-pyrrolo[2,3-b]pyridin-5-yl)oxy)-N-((4-(((2- |
| | oxaspiro[3.3]heptan-6-yl)methyl)amino)-3-nitrophenyl)sulfonyl)-4-(4- |
| | (1-bromo-6,7,8,9-tetrahydro-5H-benzo[7]annulen-5-yl)piperazin-1- |
| | yl)benzamide |
| 78 | (R)-2-((1H-pyrrolo[2,3-b]pyridin-5-yl)oxy)-4-(4-(1-bromo-3-fluoro- |
| | 6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 79 | (R)-2-((1H-pyrrolo[2,3-b]pyridin-5-yl)oxy)-N-((4-(((2- |
| | oxaspiro[3.5]nonan-7-yl)methyl)amino)-3-nitrophenyl)sulfonyl)-4-(4- |
| | (1-bromo-3-fluoro-6,7,8,9-tetrahydro-5H-benzo[7]annulen-5- |
| | yl)piperazin-1-yl)benzamide |
| 80 | (R)-2-((1H-pyrrolo[2,3-b]pyridin-5-yl)oxy)-N-((4-((2-(2-oxa-7- |
| | azaspiro[3.5]nonan-7-yl)ethyl)amino)-3-nitrophenyl)sulfonyl)-4-(4-(1- |
| | bromo-3-fluoro-6,7,8,9-tetrahydro-5H-benzo[7]annulen-5- |
| | yl)piperazin-1-yl)benzamide |
| 81 | 2-((1H-pyrrolo[2,3-b]pyridin-5-yl)oxy)-4-(4-((R)-1-bromo-3-fluoro- |
| | 6,7,8,9-tetrahydro-5H-benzo[7]annulen-5-yl)piperazin-1-yl)-N-((4- |
| | ((((3R,3aR,6aS)-hexahydrofuro[2,3-b]furan-3-yl)methyl)amino)-3- |
| | nitrophenyl)sulfonyl)benzamide |
| 82 | 2-((1H-pyrrolo[2,3-b]pyridin-5-yl)oxy)-4-(4-((R)-1-bromo-3-fluoro- |
| | 6,7,8,9-tetrahydro-5H-benzo[7]annulen-5-yl)piperazin-1-yl)-N-((4- |
| | ((((3S,3aS,6aR)-hexahydrofuro[2,3-b]furan-3-yl)methyl)amino)-3- |
| | nitrophenyl)sulfonyl)benzamide |
| 83 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((1-(oxetan-3-yl)piperidin-4- |
| | yl)methyl)amino)phenyl)sulfonyl)benzamide |
| 84 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4-((2- |
| | (4-(oxetan-3-yl)piperazin-1-yl)ethyl)amino)phenyl)sulfonyl)benzamide |
| 85 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((4- |
| | hydroxy-1-(oxetan-3-yl)piperidin-4-yl)methyl)amino)-3- |
| | nitrophenyl)sulfonyl)benzamide |
| 86 | (R)-2-((1H-pyrrolo[2,3-b]pyridin-5-yl)oxy)-4-(4-(1-chloro-3-fluoro- |
| | 6,7,8,9-tetrahydro-5H-benzo[7]annulen-5-yl)piperazin-1-yl)-N-((4- |
| | (((1-methylpiperidin-4-yl)methyl)amino)-3- |
| | nitrophenyl)sulfonyl)benzamide |
| 87 | (R)-2-((1H-pyrrolo[2,3-b]pyridin-5-yl)oxy)-4-(4-(1-chloro-3-fluoro- |
| | 6,7,8,9-tetrahydro-5H-benzo[7]annulen-5-yl)piperazin-1-yl)-N-((3- |
| | nitro-4-(((tetrahydro-2H-pyran-4- |
| | yl)methyl)amino)phenyl)sulfonyl)benzamide |
| 88 | (R)-2-((1H-pyrrolo[2,3-b]pyridin-5-yl)oxy)-4-(4-(1-chloro-3-fluoro- |
| | 6,7,8,9-tetrahydro-5H-benzo[7]annulen-5-yl)piperazin-1-yl)-N-((4- |
| | (((4-hydroxy-1-(oxetan-3-yl)piperidin-4-yl)methyl)amino)-3- |
| | nitrophenyl)sulfonyl)benzamide |
| 89 | (R)-2-((1H-pyrrolo[2,3-b]pyridin-5-yl)oxy)-4-(4-(1-fluoro-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 90 | (R)-2-((1H-pyrrolo[2,3-b]pyridin-5-yl)oxy)-4-(4-(1-methyl-6,7,8,9- |
| | tetrahydro-5H-benzo[7]annulen-5-yl)piperazin-1-yl)-N-((3-nitro-4- |
| | (((tetrahydro-2H-pyran-4-yl)methyl)amino)phenyl)sulfonyl)benzamide |
| 91 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(6-(1,2,3,4- |
| | tetrahydronaphthalen-1-yl)-2,6-diazaspiro[3.4]octan-2-yl)benzamide |
| 92 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(7-(1,2,3,4- |
| | tetrahydronaphthalen-1-yl)-2,7-diazaspiro[3.5]nonan-2-yl)benzamide |
| 93 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(2-(6,7,8,9- |
| | tetrahydro-5H-benzo[7]annulen-5-yl)-2,7-diazaspiro[3.5]nonan-7- |
| | yl)benzamide |
| 94 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(2-(1,2,3,4- |
| | tetrahydronaphthalen-1-yl)-2,7-diazaspiro[3.5]nonan-7-yl)benzamide |
| 95 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(6-(6,7,8,9- |
| | tetrahydro-5H-benzo[7]annulen-5-yl)-2,6-diazaspiro[3.3]heptan-2- |
| | yl)benzamide |
| 96 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(6-(1,2,3,4- |
| | tetrahydronaphthalen-1-yl)-2,6-diazaspiro[3.3]heptan-2-yl)benzamide |
| 97 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(7-(6,7,8,9- |
| | tetrahydro-5H-benzo[7]annulen-5-yl)-2,7-diazaspiro[3.5]nonan-2- |
| | yl)benzamide |
| 98 | 2-((1H-pyrrolo[2,3-b]pyridin-5-yl)oxy)-N-((3-nitro-4-(((tetrahydro- |
| | 2H-pyran-4-yl)methyl)amino)phenyl)sulfonyl)-4-(6-(6,7,8,9- |
| | tetrahydro-5H-benzo[7]annulen-5-yl)-2,6-diazaspiro[3.4]octan-2- |
| | yl)benzamide |
| 99 | (R)-2-((1H-pyrrolo[2,3-b]pyridin-5-yl)oxy)-N-((4-(((7- |
| | oxaspiro[3.5]nonan-2-yl)methyl)amino)-3-nitrophenyl)sulfonyl)-4-(4- |
| | (1-chloro-6,7,8,9-tetrahydro-5H-benzo[7]annulen-5-yl)piperazin-1- |
| | yl)benzamide |
| 100 | tert-butyl (R)-2-(((4-(N-(2-((1H-pyrrolo[2,3-b]pyridin-5-yl)oxy)-4-(4- |
| | (1-chloro-6,7,8,9-tetrahydro-5H-benzo[7]annulen-5-yl)piperazin-1- |
| | yl)benzoyl)sulfamoyl)-2-nitrophenyl)amino)methyl)-7- |
| | azaspiro[3.5]nonane-7-carboxylate |
| 101 | 2-((1H-pyrrolo[2,3-b]pyridin-5-yl)oxy)-4-(4-((R)-1-chloro-6,7,8,9- |
| | tetrahydro-5H-benzo[7]annulen-5-yl)piperazin-1-yl)-N-((4-(((3,3- |
| | dimethyltetrahydro-2H-pyran-4-yl)methyl)amino)-3- |
| | nitrophenyl)sulfonyl)benzamide |
| 102 | 2-((1H-pyrrolo[2,3-b]pyridin-5-yl)oxy)-4-(4-((R)-1-chloro-6,7,8,9- |
| | tetrahydro-5H-benzo[7]annulen-5-yl)piperazin-1-yl)-N-((4-(((2,2- |
| | dimethyltetrahydro-2H-pyran-4-yl)methyl)amino)-3- |
| | nitrophenyl)sulfonyl)benzamide |
| 103 | tert-butyl 5-(((4-(N-(2-((1H-pyrrolo[2,3-b]pyridin-5-yl)oxy)-4-(4-((R)- |
| | 1-chloro-6,7,8,9-tetrahydro-5H-benzo[7]annulen-5-yl)piperazin-1- |
| | yl)benzoyl)sulfamoyl)-2-nitrophenyl)amino)methyl)-2- |
| | azabicyclo[2.2.1]heptane-2-carboxylate |
| 104 | (R)-2-((1H-pyrrolo[2,3-b]pyridin-5-yl)oxy)-N-((4-(((7- |
| | azaspiro[3.5]nonan-2-yl)methyl)amino)-3-nitrophenyl)sulfonyl)-4-(4- |
| | (1-chloro-6,7,8,9-tetrahydro-5H-benzo[7]annulen-5-yl)piperazin-1- |
| | yl)benzamide |
| 105 | 2-((1H-pyrrolo[2,3-b]pyridin-5-yl)oxy)-N-((4-(((2- |
| | azabicyclo[2.2.1]heptan-5-yl)methyl)amino)-3-nitrophenyl)sulfonyl)- |
| | 4-(4-((R)-1-chloro-6,7,8,9-tetrahydro-5H-benzo[7]annulen-5- |
| | yl)piperazin-1-yl)benzamide |
| 106 | 2-((1H-pyrrolo[2,3-b]pyridin-5-yl)oxy)-4-(4-((R)-1-chloro-6,7,8,9- |
| | tetrahydro-5H-benzo[7]annulen-5-yl)piperazin-1-yl)-N-((4-(((2- |
| | methyl-2-azabicyclo[2.2.1]heptan-5-yl)methyl)amino)-3- |
| | nitrophenyl)sulfonyl)benzamide |
| 107 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | (methylsulfonyl)-7-azaspiro[3.5]nonan-2-yl)methyl)amino)-3- |
| | nitrophenyl)sulfonyl)benzamide |
| 108 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | methyl-7-azaspiro[3.5]nonan-2-yl)methyl)amino)-3- |
| | nitrophenyl)sulfonyl)benzamide |
| 109 | tert-butyl (R)-(2-(2-(((4-(N-(2-((1H-pyrrolo[2,3-b]pyridin-5-yl)oxy)-4- |
| | (4-(1-chloro-6,7,8,9-tetrahydro-5H-benzo[7]annulen-5-yl)piperazin-1- |
| | yl)benzoyl)sulfamoyl)-2-nitrophenyl)amino)methyl)-7- |
| | azaspiro[3.5]nonan-7-yl)ethyl)carbamate |
| 110 | (R)-2-((1H-pyrrolo[2,3-b]pyridin-5-yl)oxy)-N-((4-(((7-(2-aminoethyl)- |
| | 7-azaspiro[3.5]nonan-2-yl)methyl)amino)-3-nitrophenyl)sulfonyl)-4- |
| | (4-(1-chloro-6,7,8,9-tetrahydro-5H-benzo[7]annulen-5-yl)piperazin-1- |
| | yl)benzamide |
| 111 | (R)-2-((1H-pyrrolo[2,3-b]pyridin-5-yl)oxy)-N-((4-(((7-acetyl-7- |
| | azaspiro[3.5]nonan-2-yl)methyl)amino)-3-nitrophenyl)sulfonyl)-4-(4- |
| | (1-chloro-6,7,8,9-tetrahydro-5H-benzo[7]annulen-5-yl)piperazin-1- |
| | yl)benzamide |
| 112 | (R)-2-((1H-pyrrolo[2,3-b]pyridin-5-yl)oxy)-N-((4-(((7-(2- |
| | acetamidoethyl)-7-azaspiro[3.5]nonan-2-yl)methyl)amino)-3- |
| | nitrophenyl)sulfonyl)-4-(4-(1-chloro-6,7,8,9-tetrahydro-5H- |
| | benzo[7]annulen-5-yl)piperazin-1-yl)benzamide |
| 113 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7-(2,2- |
| | difluoroethyl)-7-azaspiro[3.5]nonan-2-yl)methyl)amino)-3- |
| | nitrophenyl)sulfonyl)benzamide |
| 114 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7-(2- |
| | fluoroethyl)-7-azaspiro[3.5]nonan-2-yl)methyl)amino)-3- |
| | nitrophenyl)sulfonyl)benzamide |
| 115 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | (((7-(2,2,2-trifluoroethyl)-7-azaspiro[3.5]nonan-2- |
| | yl)methyl)amino)phenyl)sulfonyl)benzamide |
| 116 | tert-butyl (R)-2-(((4-(N-(4-(4-(1-chloro-6,7,8,9-tetrahydro-5H- |
| | benzo[7]annulen-5-yl)piperazin-1-yl)benzoyl)sulfamoyl)-2- |
| | nitrophenyl)amino)methyl)-7-azaspiro[3.5]nonane-7-carboxylate |
| 117 | (R)-N-((4-(((7-azaspiro[3.5]nonan-2-yl)methyl)amino)-3- |
| | nitrophenyl)sulfonyl)-4-(4-(1-chloro-6,7,8,9-tetrahydro-5H- |
| | benzo[7]annulen-5-yl)piperazin-1-yl)benzamide |
| 118 | (R)-N-((4-(((7-acetyl-7-azaspiro[3.5]nonan-2-yl)methyl)amino)-3- |
| | nitrophenyl)sulfonyl)-4-(4-(1-chloro-6,7,8,9-tetrahydro-5H- |
| | benzo[7]annulen-5-yl)piperazin-1-yl)benzamide |
| 119 | tert-butyl (R)-(2-(2-(((4-(N-(4-(4-(1-chloro-6,7,8,9-tetrahydro-5H- |
| | benzo[7]annulen-5-yl)piperazin-1-yl)benzoyl)sulfamoyl)-2- |
| | nitrophenyl)amino)methyl)-7-azaspiro[3.5]nonan-7-yl)ethyl)carbamate |
| 120 | (R)-N-((4-(((7-(2-aminoethyl)-7-azaspiro[3.5]nonan-2- |
| | yl)methyl)amino)-3-nitrophenyl)sulfonyl)-4-(4-(1-chloro-6,7,8,9- |
| | tetrahydro-5H-benzo[7]annulen-5-yl)piperazin-1-yl)benzamide |
| 121 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7-(2- |
| | hydroxyethyl)-7-azaspiro[3.5]nonan-2-yl)methyl)amino)-3- |
| | nitrophenyl)sulfonyl)benzamide |
| 122 | ethyl (R)-2-(2-(((4-(N-(2-((1H-pyrrolo[2,3-b]pyridin-5-yl)oxy)-4-(4- |
| | (1-chloro-6,7,8,9-tetrahydro-5H-benzo[7]annulen-5-yl)piperazin-1- |
| | yl)benzoyl)sulfamoyl)-2-nitrophenyl)amino)methyl)-7- |
| | azaspiro[3.5]nonan-7-yl)acetate |
| 123 | (R)-2-(2-(((4-(N-(2-((1H-pyrrolo[2,3-b]pyridin-5-yl)oxy)-4-(4-(1- |
| | chloro-6,7,8,9-tetrahydro-5H-benzo[7]annulen-5-yl)piperazin-1- |
| | yl)benzoyl)sulfamoyl)-2-nitrophenyl)amino)methyl)-7- |
| | azaspiro[3.5]nonan-7-yl)acetic acid |
| 124 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | isobutyryl-7-azaspiro[3.5]nonan-2-yl)methyl)amino)-3- |
| | nitrophenyl)sulfonyl)benzamide |
| 125 | 2-((1H-pyrrolo[2,3-b]pyridin-5-yl)oxy)-N-((4-(((7-(L-valyl)-7- |
| | azaspiro[3.5]nonan-2-yl)methyl)amino)-3-nitrophenyl)sulfonyl)-4-(4- |
| | ((R)-1-chloro-6,7,8,9-tetrahydro-5H-benzo[7]annulen-5-yl)piperazin- |
| | 1-yl)benzamide |
| 126 | (R)-3-(2-(((4-(N-(2-((1H-pyrrolo[2,3-b]pyridin-5-yl)oxy)-4-(4-(1- |
| | chloro-6,7,8,9-tetrahydro-5H-benzo[7]annulen-5-yl)piperazin-1- |
| | yl)benzoyl)sulfamoyl)-2-nitrophenyl)amino)methyl)-7- |
| | azaspiro[3.5]nonan-7-yl)propanoic acid |
| 127 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | (cyclopropylsulfonyl)-7-azaspiro[3.5]nonan-2-yl)methyl)amino)-3- |
| | nitrophenyl)sulfonyl)benzamide |
| 128 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | (isopropylsulfonyl)-7-azaspiro[3.5]nonan-2-yl)methyl)amino)-3- |
| | nitrophenyl)sulfonyl)benzamide |
| 129 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | (cyclopropylmethyl)-7-azaspiro[3.5]nonan-2-yl)methyl)amino)-3- |
| | nitrophenyl)sulfonyl)benzamide |
| 130 | ethyl (R)-3-(2-(((4-(N-(2-((1H-pyrrolo[2,3-b]pyridin-5-yl)oxy)-4-(4- |
| | (1-chloro-6,7,8,9-tetrahydro-5H-benzo[7]annulen-5-yl)piperazin-1- |
| | yl)benzoyl)sulfamoyl)-2-nitrophenyl)amino)methyl)-7- |
| | azaspiro[3.5]nonan-7-yl)propanoate |
| 131 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7-ethyl- |
| | 7-azaspiro[3.5]nonan-2-yl)methyl)amino)-3- |
| | nitrophenyl)sulfonyl)benzamide |
| 132 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((3-nitro-4- |
| | ((spiro[3.5]nonan-2-ylmethyl)amino)phenyl)sulfonyl)benzamide |
| 133 | (R)-2-((1H-pyrrolo[2,3-b]pyridin-5-yl)oxy)-N-((4-(((8,11- |
| | dioxadispiro[3.2.47.24]tridecan-2-yl)methyl)amino)-3- |
| | nitrophenyl)sulfonyl)-4-(4-(1-chloro-6,7,8,9-tetrahydro-5H- |
| | benzo[7]annulen-5-yl)piperazin-1-yl)benzamide |
| 134 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7- |
| | hydroxyspiro[3.5]nonan-2-yl)methyl)amino)-3- |
| | nitrophenyl)sulfonyl)benzamide |
| 135 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7,7- |
| | difluorospiro[3.5]nonan-2-yl)methyl)amino)-3- |
| | nitrophenyl)sulfonyl)benzamide |
| 136 | (R)-2-((1H-pyrrolo[2,3-b]pyridin-5-yl)oxy)-4-(4-(1-chloro-6,7,8,9- |
| | tetrahydro-5H-benzo[7]annulen-5-yl)piperazin-1-yl)-N-((4-(((7,7- |
| | dimethylspiro[3.5]nonan-2-yl)methyl)amino)-3- |
| | nitrophenyl)sulfonyl)benzamide |

or a tautomer, a stereoisomer, or a pharmaceutically acceptable salt thereof.

75. A pharmaceutical composition comprising i) a compound according to any one of claims 1-74, a tautomer, a stereoisomer, or a pharmaceutically acceptable salt thereof, and ii) a pharmaceutically acceptable excipient or pharmaceutically acceptable carrier.

76. A method of modulating the level or activity of BCL-2 or BCL-2/BCL-XL in a cell, comprising exposing the cell to a compound, a tautomer, a stereoisomer, or a pharmaceutically acceptable salt thereof, as claimed in any one of claims 1-74, or the pharmaceutical composition as claimed in claim 75.

77. A method of treating a BCL-2 or BCL-2/BCL-XL associated disease, disorder or condition in a subject in need thereof, comprising administering to the subject a therapeutically effective amount of the compound, a tautomer, a stereoisomer, or a pharmaceutically acceptable salt thereof, as claimed in any one of claims 1-74, or the pharmaceutical composition as claimed in claim 75.

78. The method of claim 77, wherein the BCL-2 or BCL-2/BCL-XL associated disease, disorder or condition is related to an increased level or activity of BCL-2 protein or BCL-2/BCL-XL proteins.

79. The method of claim 78, wherein the disease, disorder or condition is selected from the group consisting of leukemia, Hodgkin lymphoma, Non-Hodgkin lymphoma, mantle cell lymphomas, gastro-intestinal cancer, gastric cancer, vascular cancer, biliary carcinomas, pancreatic cancer, colorectal cancer, esophageal cancer, hepatocellular cancer, melanoma, myeloma, oral cancer, ovarian cancer, small cell lung cancer, non-small cell lung cancer, myeloma, prostate cancer, bladder cancer, brain cancer, breast cancer, bone marrow cancer, cervical cancer and spleen cancer.

80. The method of claim 79, wherein the leukemia is selected from the group consisting of lymphatic leukemia, lymphocytic leukemia, chronic lymphocytic leukemia, small lymphocytic lymphoma, diffuse large B-cell lymphoma, acute myeloid leukemia, lymphoblastic leukemia, follicular lymphoma, lymphoid malignancies of T-cell or B-cell origin, myelogenous leukemia, granulocytic leukemia, polycythemia vera, erythremia.

81. The method of any one of claims 77-80, wherein the compound, a tautomer, a stereoisomer, or a pharmaceutically acceptable salt thereof, as claimed in any one of claims 1-74, or the pharmaceutical composition as claimed in claim 75 is administered simultaneously, separately or sequentially with a second therapy.

82. The method of claim 81, wherein the second therapy is chemotherapy or immunotherapy.

83. The method of claim 81, wherein the second therapy is selected from the group consisting of a chemotherapeutic agent, an anti-tumor agent, a radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, and cytokines.

84. The method of claim 81, wherein the second therapy is a BTK inhibitor, a BCR-ABL inhibitor, a JAK3 inhibitor, or a PARP inhibitor.
